(19) United States
(12) Patent Application Publication
(10) Pub. No.: US 2026/0207752 A1
(43) Pub. Date: Jul. 23, 2026

(54) ASGPR-BINDING COMPOUNDS FOR THE DELIVERY OF OLIGONUCLEOTIDES

(71) Applicant: AVILAR THERAPEUTICS, INC., Waltham, MA (US)

(72) Inventors: Hu Liu, Newton, MA (US); Srinivasa Karra, Pembroke, MA (US); Jason Allan Wiles, Madison, CT (US)

(73) Assignee: AVILAR THERAPEUTICS, INC., Waltham, MA (US)

(21) Appl. No.: 19/572,253

(22) Filed: Mar. 19, 2026

Related U.S. Application Data
(63) Continuation of application No. PCT/US2024/047557, filed on Sep. 19, 2024.
(60) Provisional application No. 63/539,206, filed on Sep. 19, 2023.
(60) Provisional application No. 63/637,788, filed on Apr. 23, 2024.

(51) Int. Cl. A61K 47/54

(52) U.S. Cl. CPC A61K 47/549; A61K 47/545; A61K 47/548

(57) ABSTRACT

This invention provides Therapeutic Oligonucleotide delivering compounds, compositions, and methods for the treatment of disorders which respond to delivery of the Therapeutic Oligonucleotide. The Therapeutic Oligonucleotide delivering compounds have an asialoglycoprotein receptor (ASGPR) Binding Ligand bound to a Therapeutic Oligonucleotide through a Linker and a Cleavable Moiety for the delivery of the Therapeutic Oligonucleotide to the liver.

### CROSS-REFERENCE TO RELATED APPLICATIONS

This application is a continuation of International Patent Application No. PCT/US2024/047557, filed Sep. 19, 2024, which claims the benefit of U.S. Provisional Application No. 63/539,206, filed Sep. 19, 2023, and U.S. Provisional Application No. 63/637,788, filed Apr. 23, 2024. The entirety of each of these applications is hereby incorporated by reference for all purposes.

### INCORPORATION BY REFERENCE

The contents of the XML file named “19121-034WO1_ST26.xml” which was created on Sep. 19, 2024, and is 464,993 bytes in size, are hereby incorporated by reference in their entirety.

### FIELD OF THE INVENTION

This invention provides Therapeutic Oligonucleotide delivering compounds, compositions, and methods for the treatment of disorders which respond to delivery of the Therapeutic Oligonucleotide. The Therapeutic Oligonucleotide delivering compounds have an asialoglycoprotein receptor (ASGPR) Binding Ligand bound to a Therapeutic Oligonucleotide through a Linker and a Cleavable Moiety for the delivery of the Therapeutic Oligonucleotide to the liver.

### BACKGROUND OF THE INVENTION

The asialoglycoprotein receptor (ASGPR) is a Ca²⁺-dependent lectin that is primarily expressed in parenchymal hepatocyte cells. The main role of ASGPR is to help regulate serum glycoprotein levels by mediating endocytosis of desialylated glycoproteins. The receptor binds ligands with a terminal galactose or N-acetylgalactosamine. Asialoglycoproteins bind to ASGPRs and are then cleared by receptor-mediated endocytosis. The receptor and the protein are dissociated in the acidic endosomal compartment and the protein is eventually degraded by lysosomes. Heterobifunctional compounds have been developed that use an ASGPR ligand to either deliver a therapeutic nucleotide to the liver or to degrade an extracellular protein.

Publications describing various ASGPR ligands include: U.S. Pat. Nos. 9,340,553; 9,617,293; 10,039,778; 10,376,531, and 10,813,942 assigned to Pfizer Inc.; Sanhueza et al. (JACS, 2017, 139, 3528); Petrov et al. (Bioorganic and Medicinal Chemistry Letters, 2018, 28, 382); WO 2018/223073 and WO 2018/223081 assigned to Pfizer Inc. and Wave Life Sciences Ltd.; WO 2024/182749 and WO 2018/223056 assigned to Wave Sciences Ltd.; Schmidt et al. (Nucleic Acids Research, 2017, 45, 2294); Huang et al. (Bioconjugate Chem. 2017, 28, 283); WO 2020/132100 assigned to The Board of Trustees of the Leland Stanford Junior University; WO 2021/142377, WO 2023/288033, WO 2024/155746, WO 2024/155747, and WO 2024/155750 assigned to Lycia Therapeutics; Banik et al. (Nature, 2020, 584, 291); WO 2023/178179, WO 2023/178202, WO 2022/192478, WO 2022/178425, WO 2021/072269, WO 2021/072246, WO 2019/199621, and WO 2019/199634, assigned to Yale University; WO 2022/084331 assigned to Sanofi; WO 2022/192478 assigned to Biohaven Therapeutics; an article from the Bertozzi group titled “LYTACs that engage the asialoglycoprotein receptor for targeted protein degradation,” (Ahn, et al. Nat. Chem. Biol. (2021)) published in the journal Nature Chemical Biology; and WO 2021/155317, WO 2022/035997, WO 2022/235699, WO 2023/009554, WO 2023/028338, WO 2024/098039, and WO 2024/182772 assigned to Avilar Therapeutics Inc.

While some progress has been made in the area of nucleotide delivery, there remains a need for additional therapeutic compounds and methods for their use and manufacture for the delivery of therapeutic nucleotides to the liver.

### SUMMARY OF THE INVENTION

Novel Therapeutic Oligonucleotide delivering compounds and their pharmaceutically acceptable salts and compositions thereof and their methods of use and manufacture are provided. The Therapeutic Oligonucleotide delivering compounds of the present invention contain an ASGPR Binding Ligand covalently attached by a Linker and a Cleavable Moiety to a Therapeutic Oligonucleotide. The ASGPR Binding Ligands used in the Therapeutic Oligonucleotide delivering compounds described herein include derivatives of six-carbon pyranose moieties, specifically galactose and talose. These two sugars, shown below, differ only in the stereochemistry of the C² substituent. The “down” C² configuration corresponds to the stereochemistry of galactose, while the C² substituent in the “up” configuration corresponds to the stereochemistry of talose. In certain embodiments the Therapeutic Oligonucleotide delivering compound of the present invention is in the Galactose stereochemistry configuration.

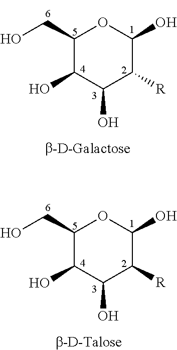

It has been discovered that certain derivatives of these sugars described herein can be used to deliver a Therapeutic Oligonucleotide to the liver. For example, in certain embodiments the Therapeutic Oligonucleotide delivering compound of the present invention results in a significantly higher proportion of the Therapeutic Oligonucleotide in the liver than in the plasma when compared to delivery of the Therapeutic Oligonucleotide separately from a compound of the present invention.

In certain aspects the Therapeutic Oligonucleotide delivering compound is administered or formulated with an oral uptake increasing agent such as 8-(N-2-hydroxy-5-chlorobenzoyl)-amino-caprylic acid (5-CNAC) or N-(8-[2-hydroxybenzoyl]amino)caprylic acid (SNAC). In certain aspects a formulation comprising a Therapeutic Oligonucleotide delivering compound is formulated for delivery to the gastrointestinal tract and optionally comprises 5-CNAC or SNAC or a salt thereof. In certain embodiments the salt of 5-CNAC is a monosodium salt or a disodium salt.

In certain aspects a Therapeutic Oligonucleotide delivering compound of Formula I, Formula II, or Formula III is provided:

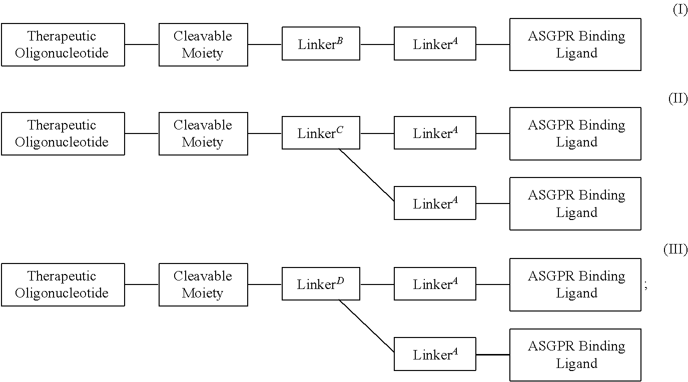

or a pharmaceutically acceptable salt thereof;
wherein

ASGPR Binding Ligand is a compound selected from:

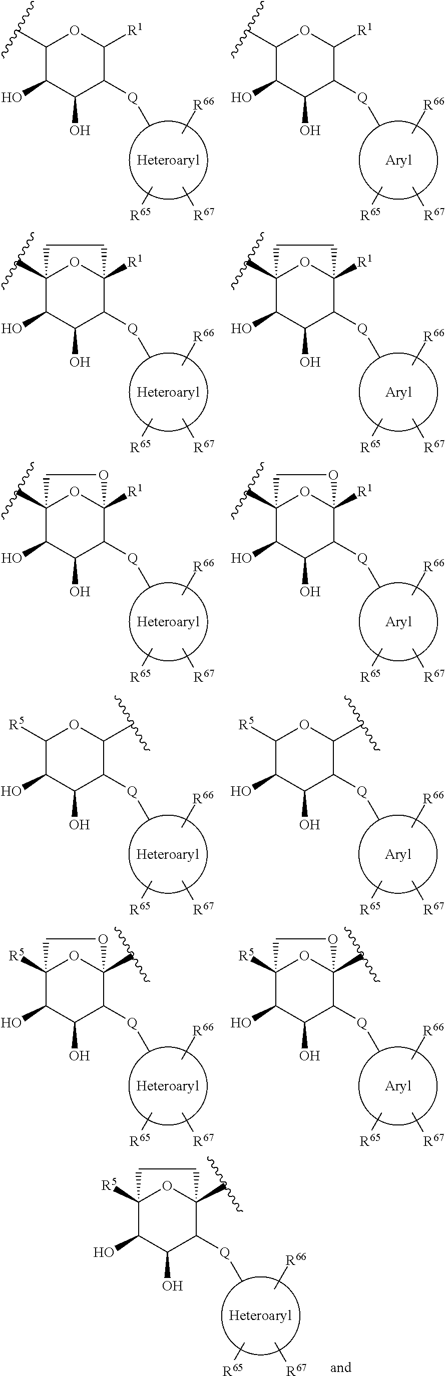

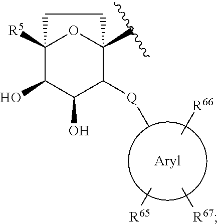

Q is —O— or —(N(R¹⁰)—;
in certain embodiments Q is —O—;
in certain embodiments Q is —N(R¹⁰)—;
in certain embodiments Q is —NH—;
R¹ and R⁵ are independently selected from hydrogen, alkyl, alkenyl, alkynyl, haloalkyl, halogen, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³;
in certain embodiments R¹ is hydrogen;
R³ at each occurrence is independently selected from the group consisting of hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from the group consisting of hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is hydrogen, alkyl, haloalkyl, arylalkyl, heteroarylalkyl, alkenyl, alkynyl, aryl, heteroaryl, heterocycle, C(O)R³, S(O)R³, C(S)R³, or S(O)₂R³;
in certain embodiments R¹⁰ is hydrogen;
R⁶⁵, R⁶⁶, and R⁶⁷ are independently selected at each occurrence from the group consisting of hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R′)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰;
R⁸⁰ is independently selected at each instance from the group consisting of alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.

Linker^{A} is a bond or a moiety that covalently links Linker^{B}, Linker^{C}, or Linker^{D} to the ASGPR Binding Ligand;
Linker^{B} is a bond or a moiety that covalently links Linker^{A} to the Therapeutic Oligonucleotide;
Linker^{C} is a chemical group that links each Linker^{A} to the Therapeutic Oligonucleotide;
Linker^{D} is a chemical group that links each Linker^{A} to the Therapeutic Oligonucleotide; and
Cleavable Moiety is a chemical moiety that can be cleaved to release the oligionucleotide; in certain aspects Cleavable Moiety is a means to release an oligionucleptide;
in certain aspects Cleavable Moiety is a bond,

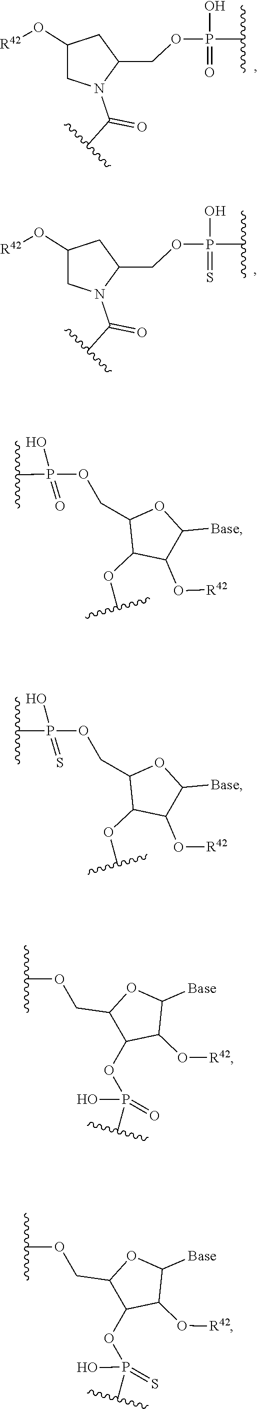

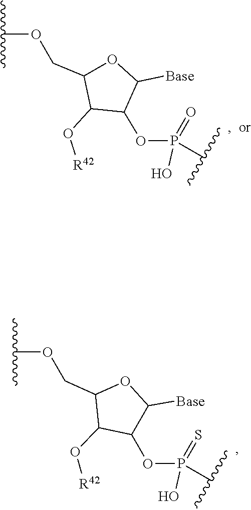

wherein the Therapeutic Oligonucleotide is attached to the —P—;
in certain embodiments the cleavable moiety comprises

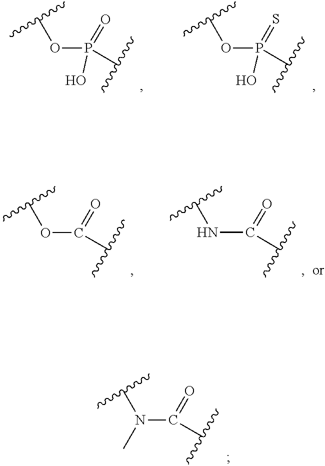

R⁴² is hydrogen, alkyl, alkyl-O-alkyl, or C(O)R⁴³;
R⁴³ is selected from the group consisting of hydrogen, alkyl, and haloalkyl;

Base is a heterocycle comprising two, three, or four nitrogen atoms, wherein the heterocycle is substituted with an oxo, amino, or carbamoyl and optionally substituted with 1, 2, or 3 substituents independently selected from alkyl, haloalkyl, and halogen;
in certain embodiments Base is selected from

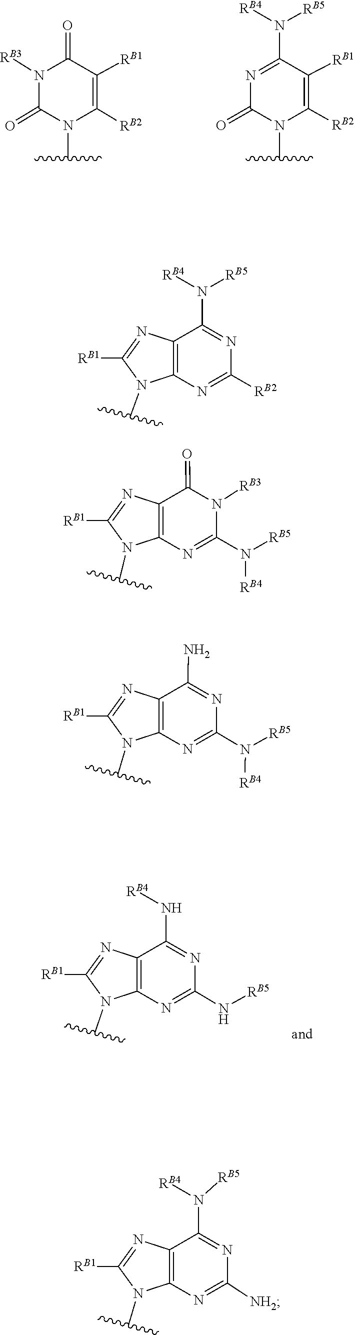

wherein
R^{B1} and R^{B2} are independently selected from H, C₁-C₄alkyl, F, C₁, and C₁-C₄haloalkyl;
R^{B3} is C₁-C₄alkyl or H;
R^{B4} and R^{B5} are independently selected from H, C₁-C₄alkyl, C(O)C₁-C₄alkyl, C₁-C₄haloalkyl, and C(O)C₁-C₄haloalkyl;
in certain embodiments Base is selected from

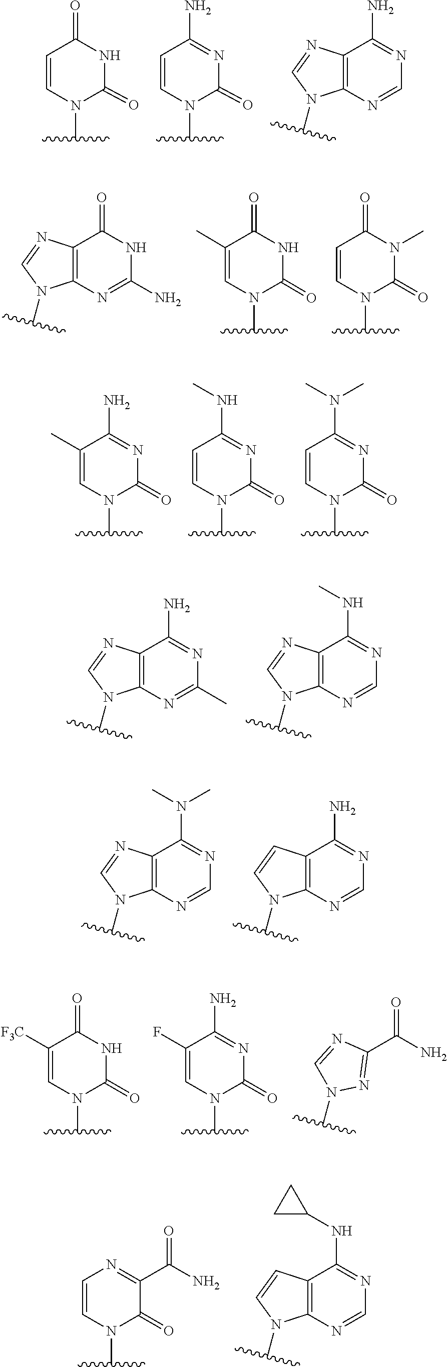

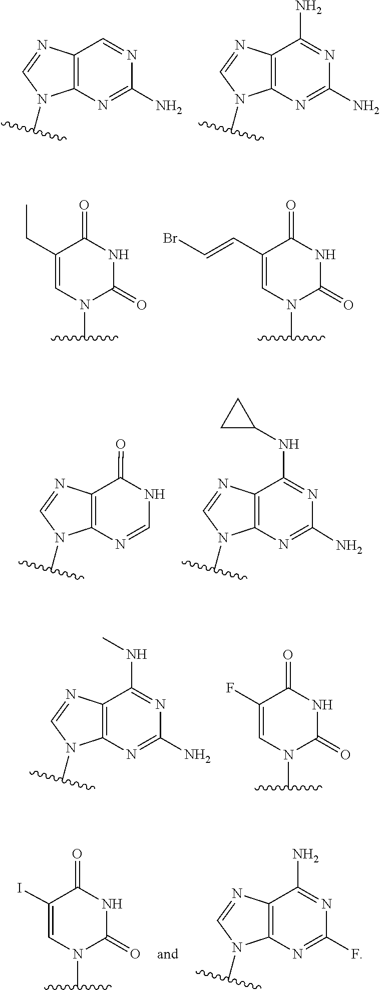

in certain embodiments Cleavable Moiety is

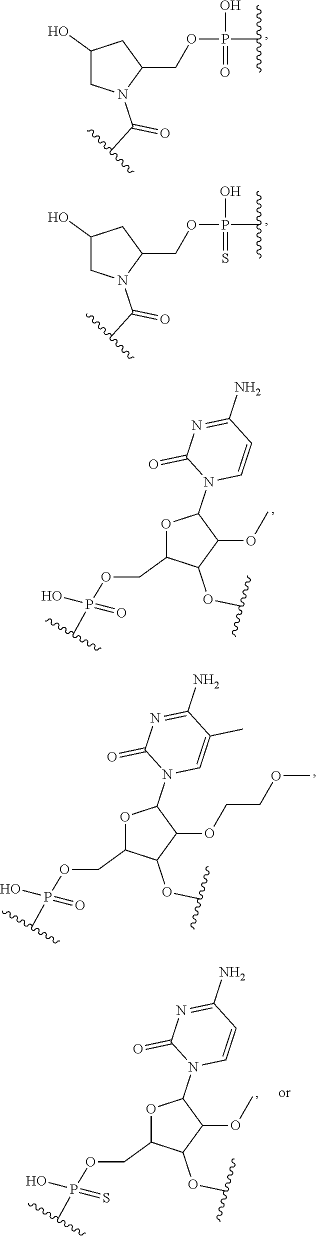 [SMILES: *C(=O)N1CC(O)CC1COP(*)(=O)O ; *C(=O)N1CC(O)CC1COP(*)(O)=S ; *OC1C(COP(*)(=O)O)OC(n2ccc(N)nc2=O)C1OC ; *OC1C(COP(*)(=O)O)OC(n2cc(C)c(N)nc2=O)C1OCCOC ; *OC1C(COP(*)(O)=S)OC(n2ccc(N)nc2=O)C1OC]

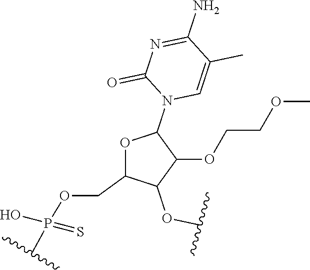 [SMILES: *OC1C(COP(*)(O)=S)OC(n2cc(C)c(N)nc2=O)C1OCCOC]

wherein the Therapeutic Oligonucleotide is attached to the —P—. In other embodiments Cleavable Moiety is

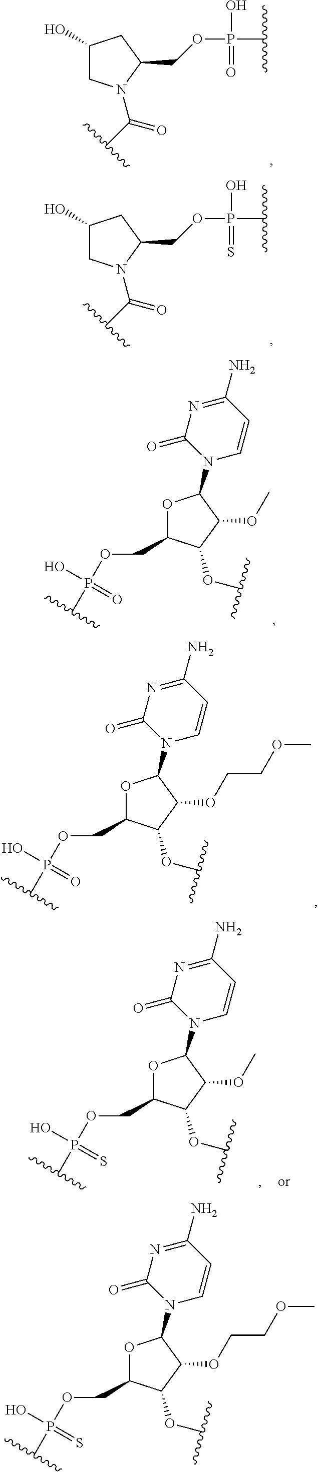 [SMILES: *C(=O)N1C[C@H](O)C[C@H]1COP(*)(=O)O ; *C(=O)N1C[C@H](O)C[C@H]1COP(*)(O)=S ; *O[C@H]1[C@@H](OC)[C@H](n2ccc(N)nc2=O)O[C@@H]1COP(*)(=O)O ; *O[C@H]1[C@@H](OCCOC)[C@H](n2ccc(N)nc2=O)O[C@@H]1COP(*)(=O)O ; *O[C@H]1[C@@H](OC)[C@H](n2ccc(N)nc2=O)O[C@@H]1COP(*)(O)=S ; *O[C@H]1[C@@H](OCCOC)[C@H](n2ccc(N)nc2=O)O[C@@H]1COP(*)(O)=S]

wherein the Therapeutic Oligonucleotide is attached to the —P—.

Therapeutic Oligonucleotide is an antisense oligonucleotide, siRNA, shRNA, DNA aptamer, RNA aptamer, miRNA, miRNA mimic, antimiRNA, DNA decoy, RNA decoy, or CpG oligonucleotide. In certain aspects Therapeutic Oligonucleotide is a means to treat a medical disorder.

In another aspect the ASGPR Binding Ligand for use in a compound of Formula I, Formula II, or Formula III is a compound selected from:

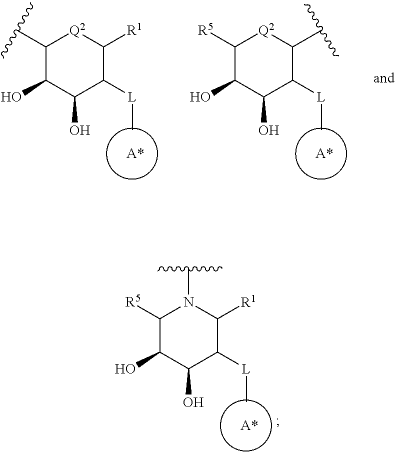

wherein
Q² is selected from —O—, —N(R¹⁰)—, S, S(O) C(R^{4a})(R^{4b}b, and S(O)₂;
L is selected from

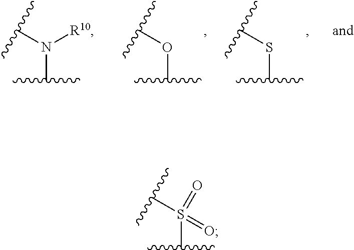

[symbol] is heteroaryl or phenyl, each of which is optionally substituted with 1 or 2 substituents independently selected from R⁶⁵;
R^{4a} is selected from hydrogen, alkyl, haloalkyl, and halogen; and
R^{4b} is selected from hydrogen, alkyl, haloalkyl, halogen, C₀-C₆alkyl-OR⁶, C₀-C₆alkyl-SR⁶, and C₀-C₆alkyl-NR⁶R⁷.

In certain aspects the Therapeutic Oligonucleotide is a nucleotide described in Table 1. In certain embodiments the Therapeutic Oligonucleotide is a nucleotide that is at least 80%, at least 85%, at least 90%, at least 95%, at least 96%, at least 97%, at least 98%, or at least 99% identical to a nucleotide described in Table 1.

In alternative embodiments, R¹ and R⁵ are independently selected from hydrogen, alkyl, alkenyl, alkynyl, haloalkyl, halogen, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, C₀-C₆alkyl-N═S(O)(R³)₂, C₀-C₆alkylN₃, or C₀-C₆alkyl-O—S(O)₂R³.

In non-limiting embodiments, Linker^{A} and Linker^{B} are independently selected from:

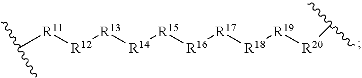

Linker^{C} is selected from:

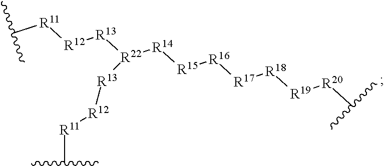

and

Linker^{D} is selected from:

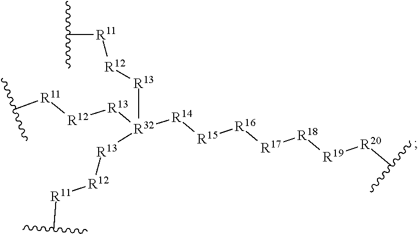

wherein:
R¹¹, R¹², R¹³, R¹⁴, R¹⁵, R¹⁶, R¹⁷, R⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻; and
all other variables are as defined herein.

In alternative embodiments, R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[C(O)—CH₂—NR⁶]ₙ—, a divalent residue of a fatty acid, a divalent residue of an unsaturated or saturated mono- or di-carboxylic acid; each of which is optionally substituted with 1, 2, 3, or 4 substituents independently selected from R²¹.

In certain embodiments Linker^{A} is bond and Linker^{B} is

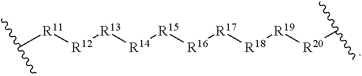

In certain embodiments Linker^{B} is bond and Linker^{A} is

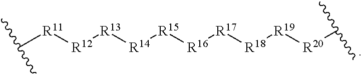

In certain embodiments Linker^{A} is bond and Linker^{B} is

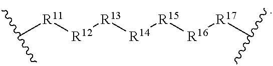

In certain embodiments Linker^{B} is bond and Linker^{A} is

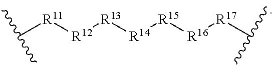

In certain embodiments Linker^{A} is bond and Linker^{B} is

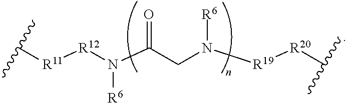

In certain embodiments Linker^{B} is bond and Linker^{A} is

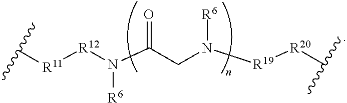

In certain embodiments Linker^{A} is bond and Linker^{B} is

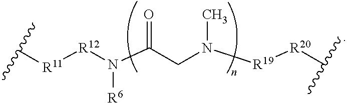

In certain embodiments Linker^{B} is bond and Linker^{A} is

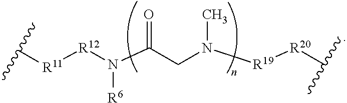

In certain embodiments Linker^{C} is

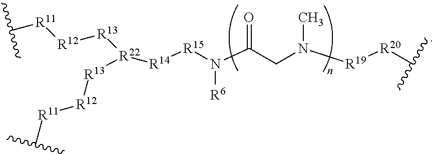

In certain embodiments Linker^{D} is

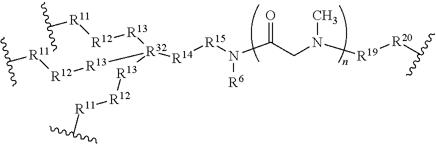

In certain embodiments ASGPR Binding Ligand is a compound selected from:

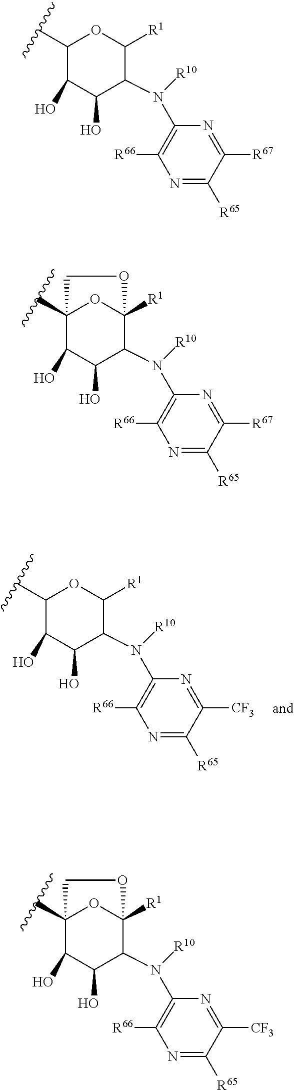

In certain embodiments the ASGPR Binding Ligand is:

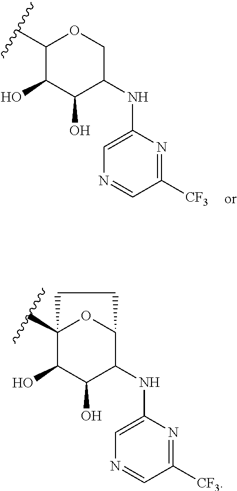 [SMILES: *C1OCC(Nc2cncc(C(F)(F)F)n2)[C@@H](O)[C@H]1O ; *[C@@]12CC[C@@H](O1)C(Nc1cncc(C(F)(F)F)n1)[C@@H](O)[C@H]2O]

In certain embodiments the ASGPR Binding Ligand is:

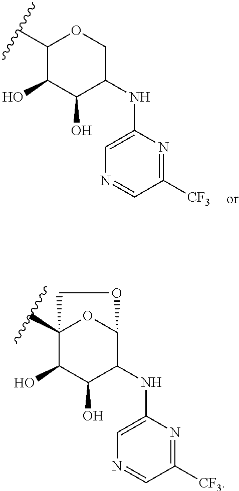 [SMILES: *C1OCC(Nc2cncc(C(F)(F)F)n2)[C@@H](O)[C@H]1O ; *[C@@]12CO[C@@H](O1)C(Nc1cncc(C(F)(F)F)n1)[C@@H](O)[C@H]2O]

In other embodiments ASGPR Binding Ligand is selected from:

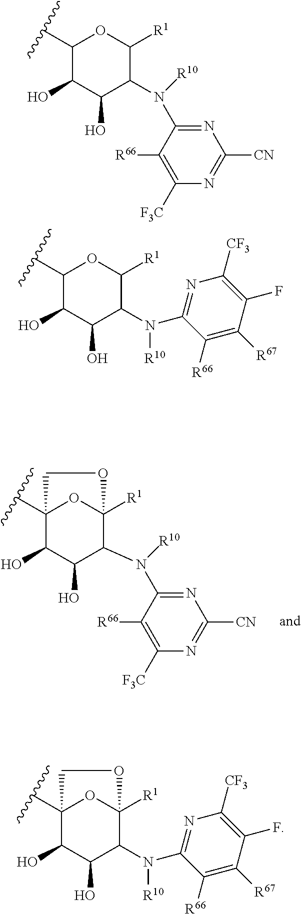

In certain embodiments, the compound of the present invention is of the formula

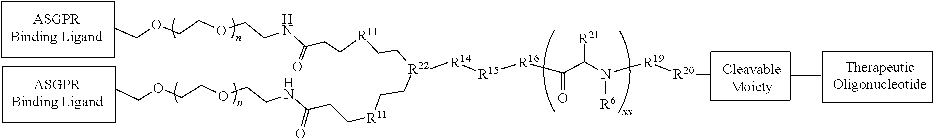

or a pharmaceutically acceptable salt thereof.

In certain embodiments, the compound of the present invention is selected from:

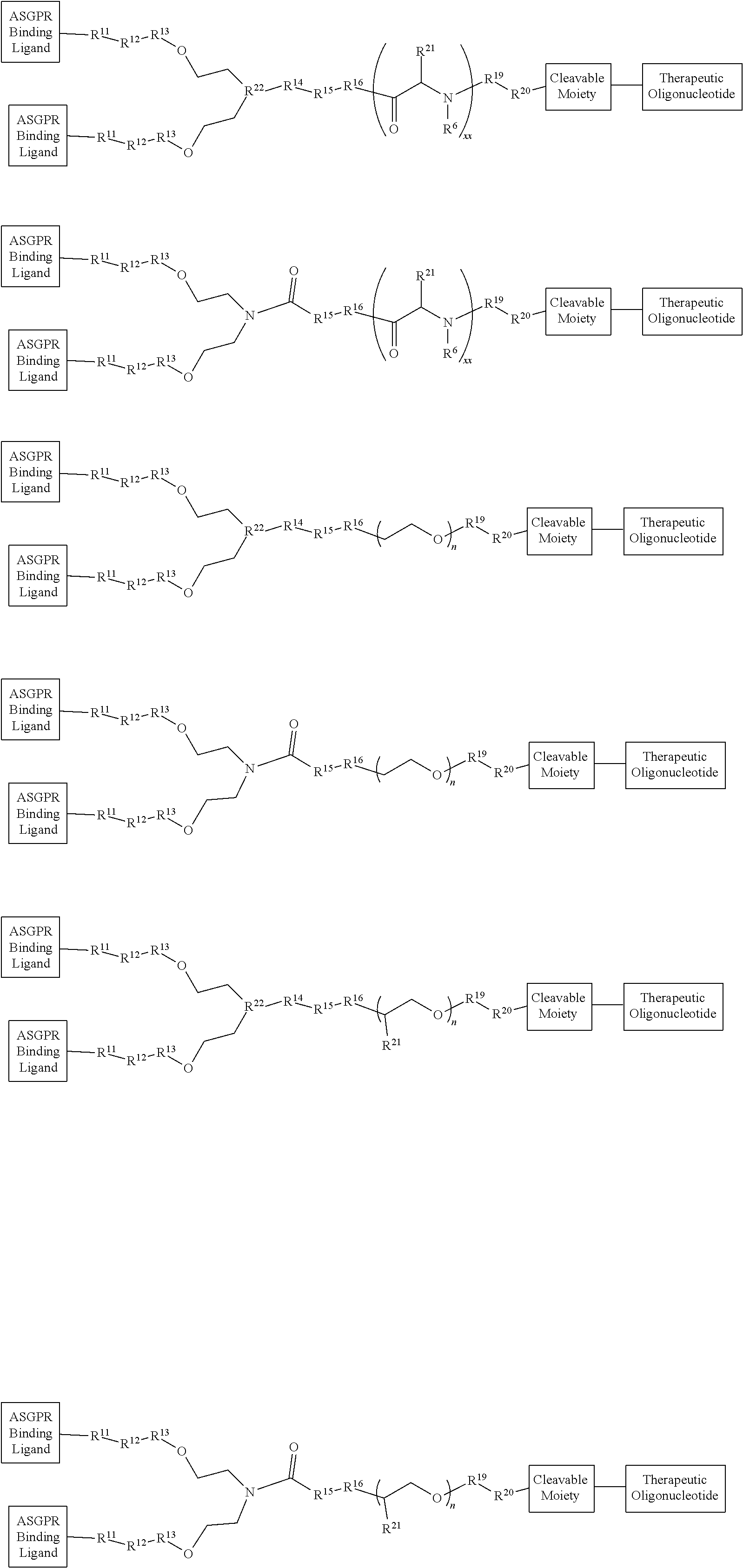

or a pharmaceutically acceptable salt thereof.

In certain embodiments, the compound of the present invention is selected from:

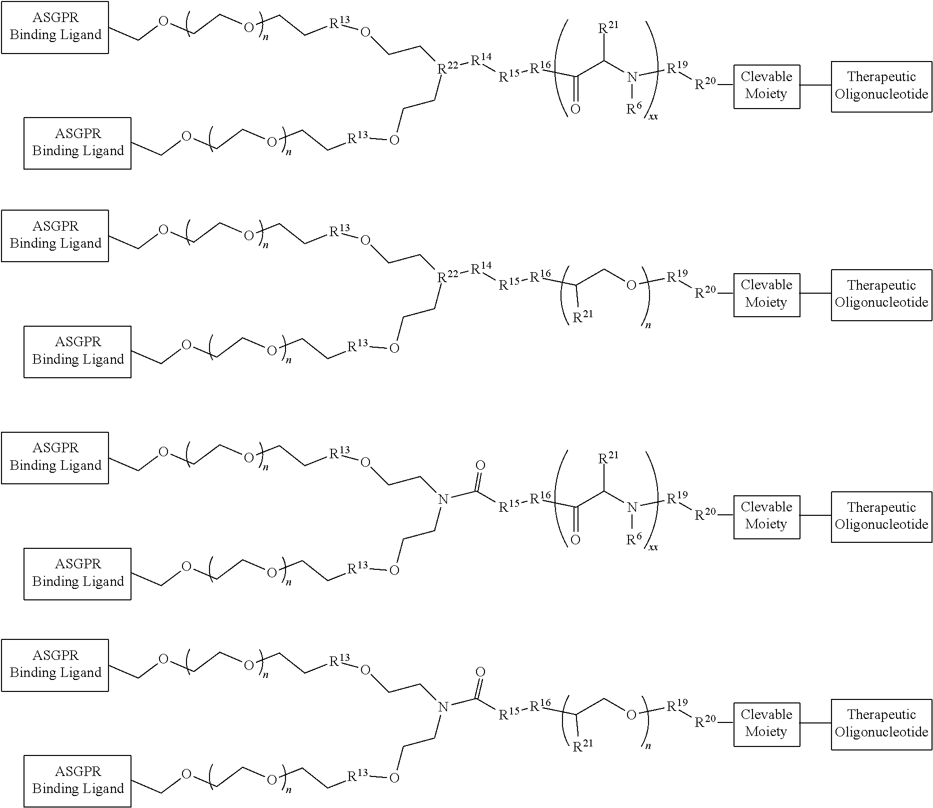

or a pharmaceutically acceptable salt thereof.

In certain embodiments, the compound of the present invention is selected from:

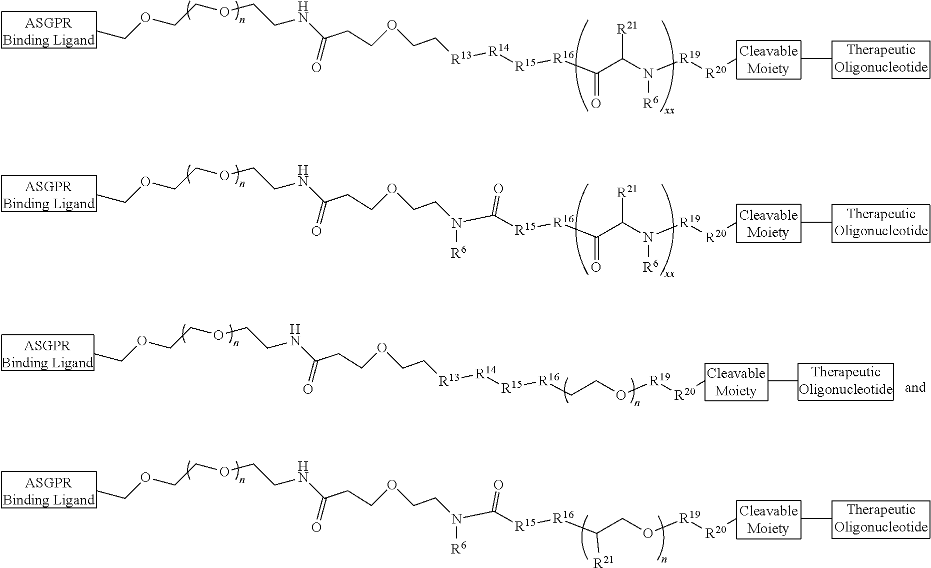

or a pharmaceutically acceptable salt thereof.

In certain embodiments, the compound of the present invention is selected from:

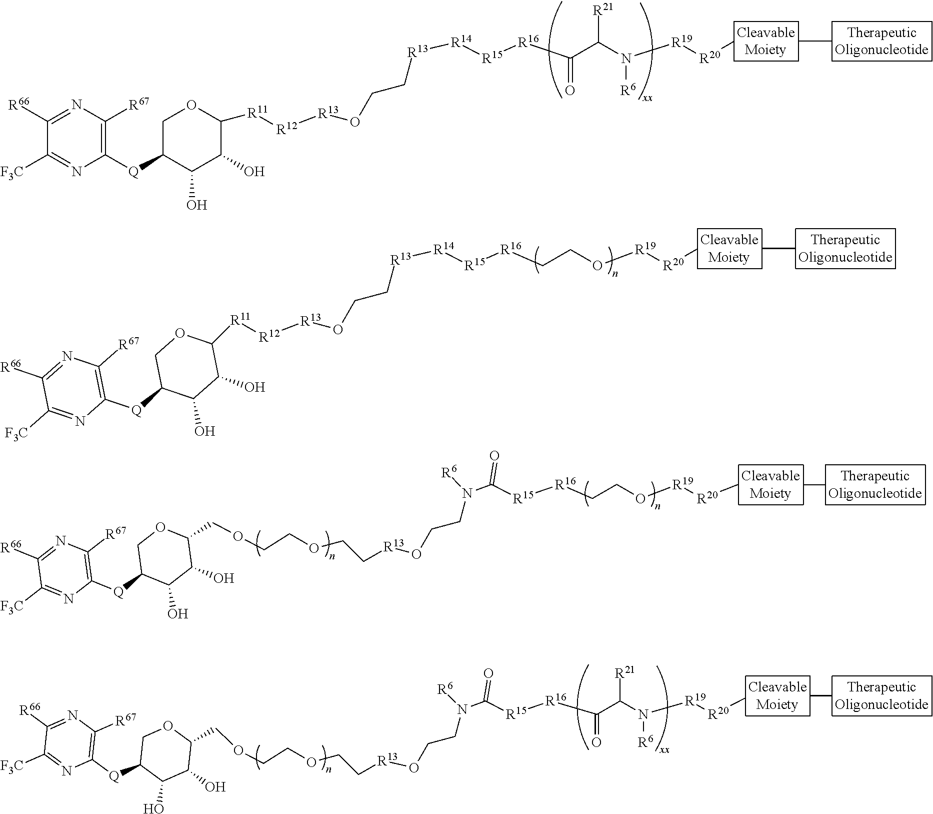

or a pharmaceutically acceptable salt thereof.

In certain embodiments, the compound of the present invention is selected from:

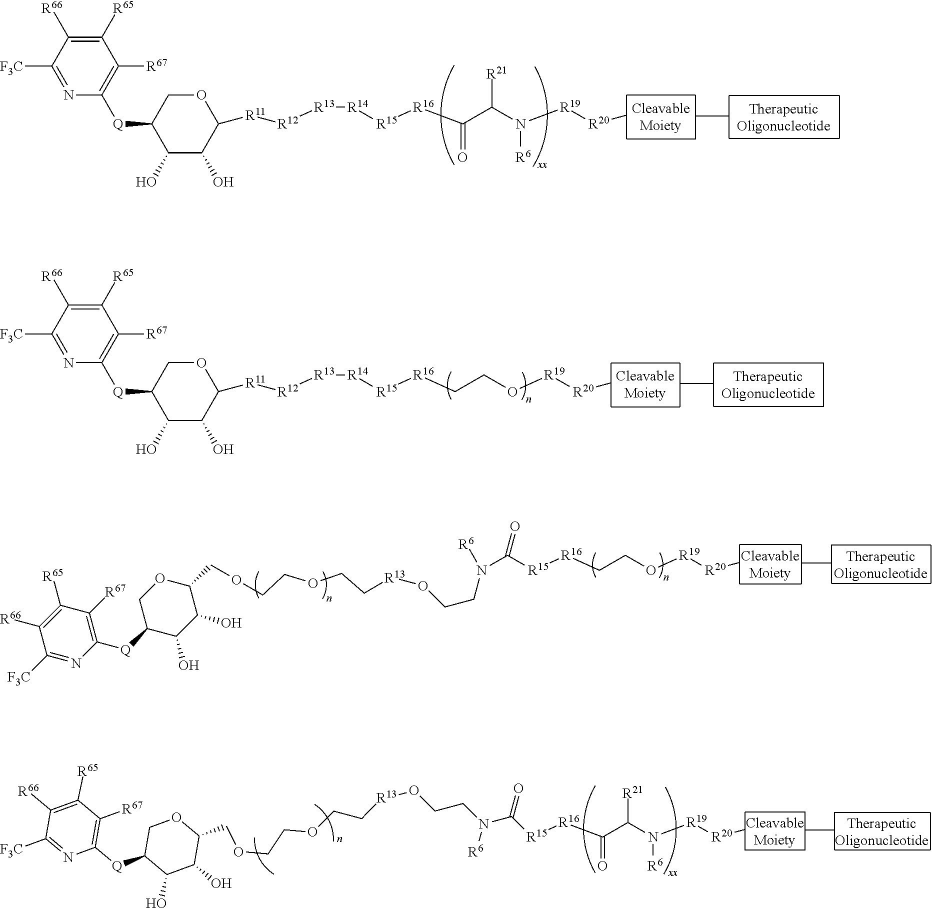

or a pharmaceutically acceptable salt thereof.

In certain embodiments, the compound of the present invention is selected from:

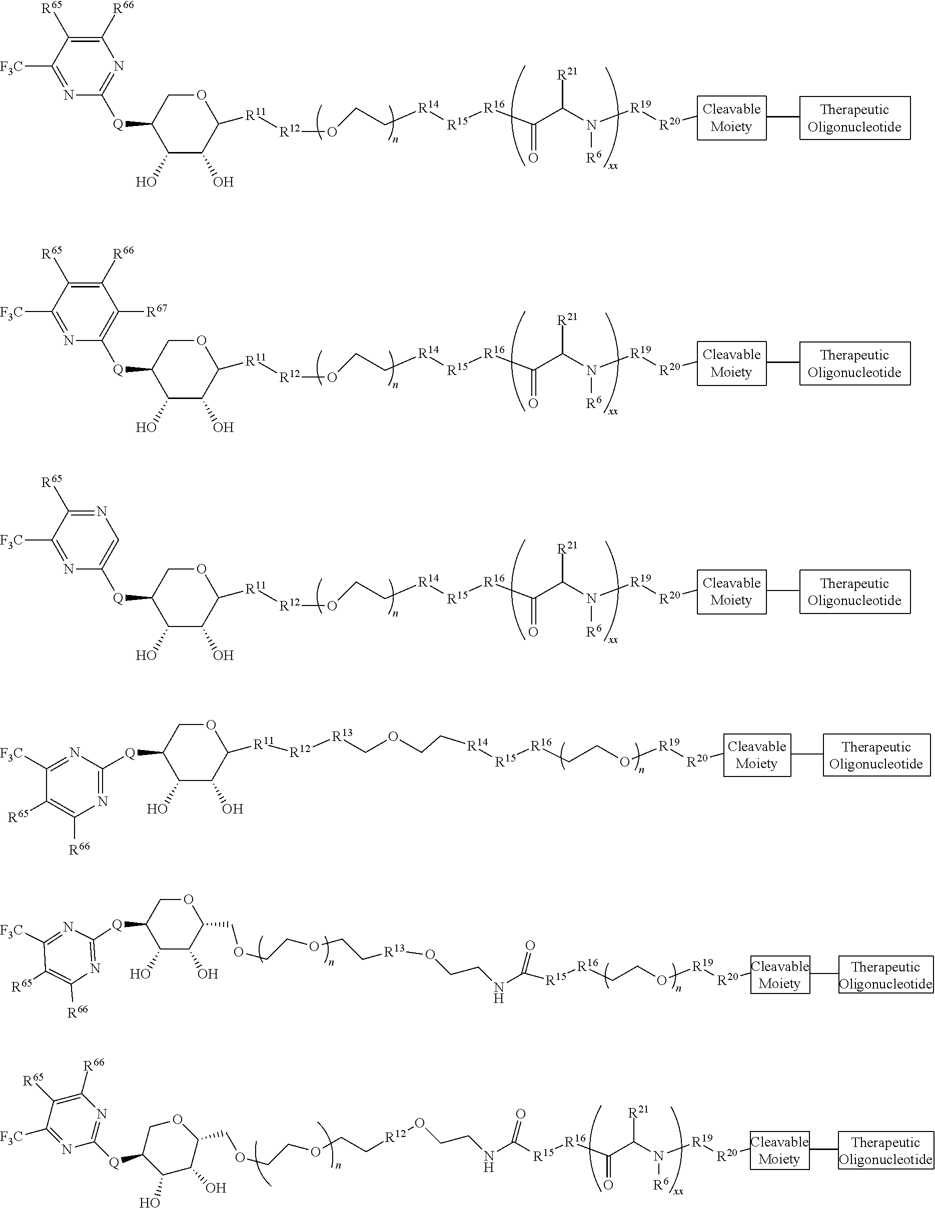

or a pharmaceutically acceptable salt thereof.

In certain embodiments, the compound of the present invention is selected from:

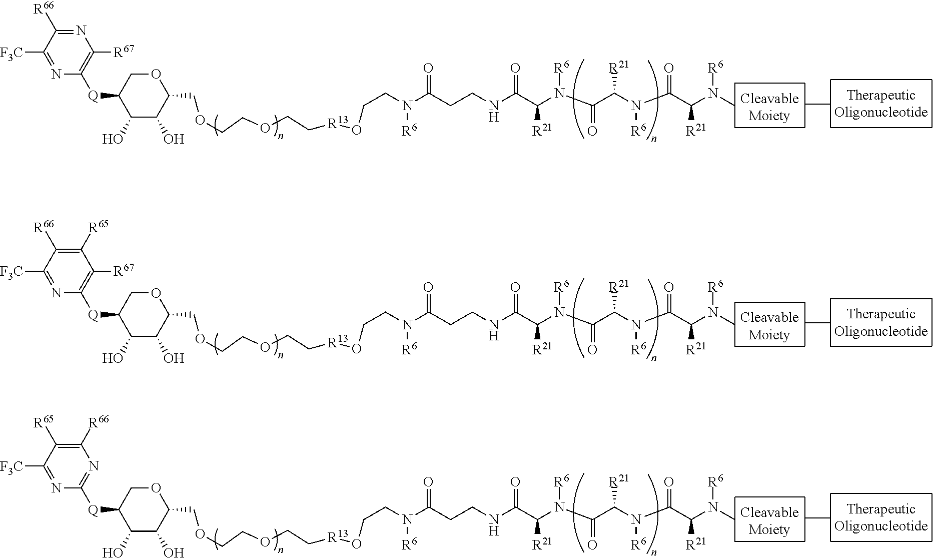

or a pharmaceutically acceptable salt thereof.

In certain embodiments, the compound of the present invention is selected from:

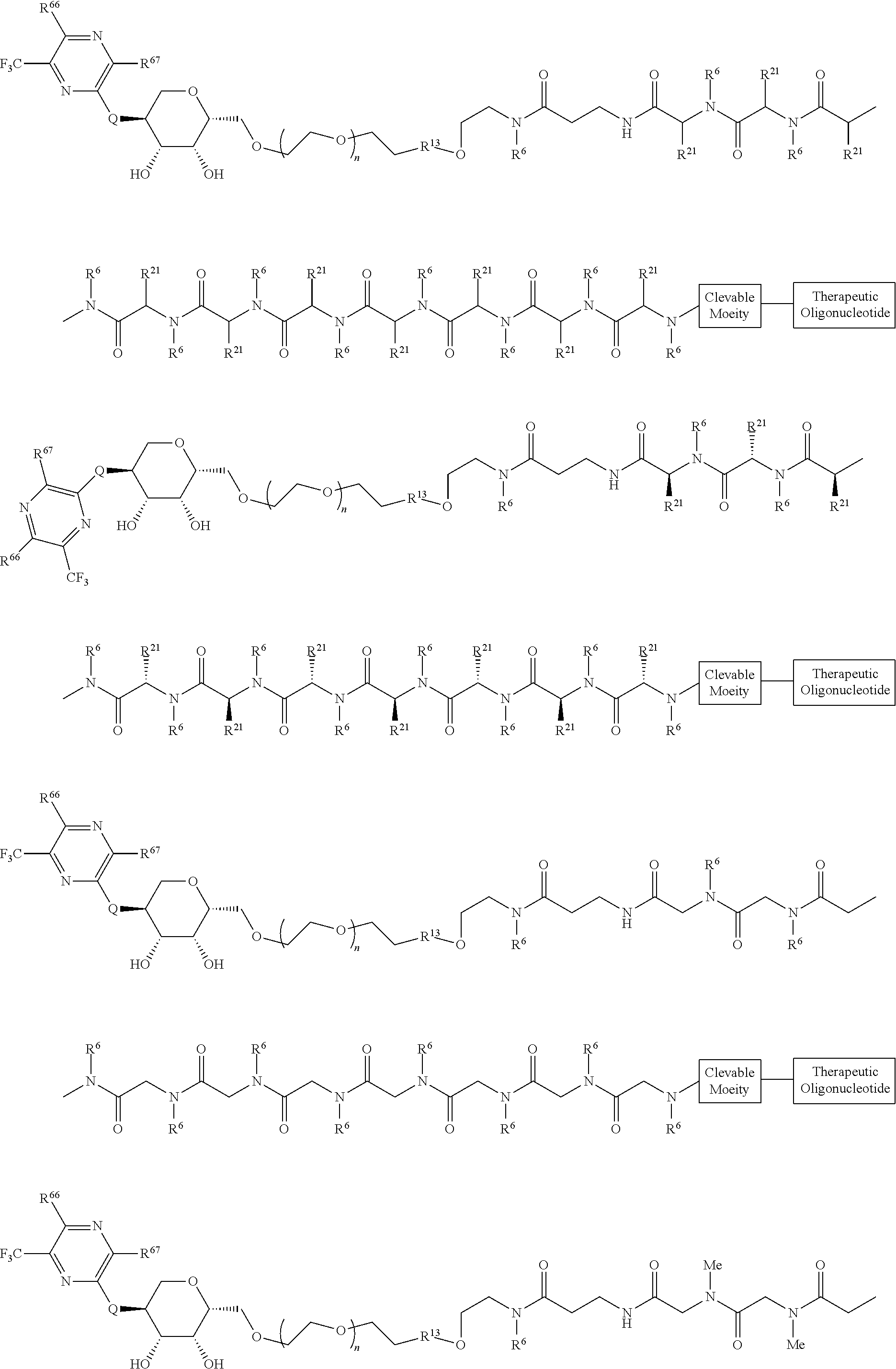

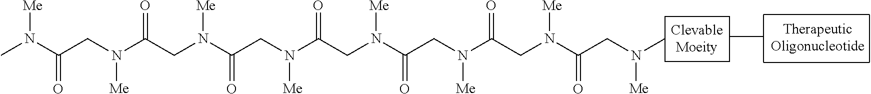

or a pharmaceutically acceptable salt thereof.

In certain embodiments, the compound of the present invention is selected from:

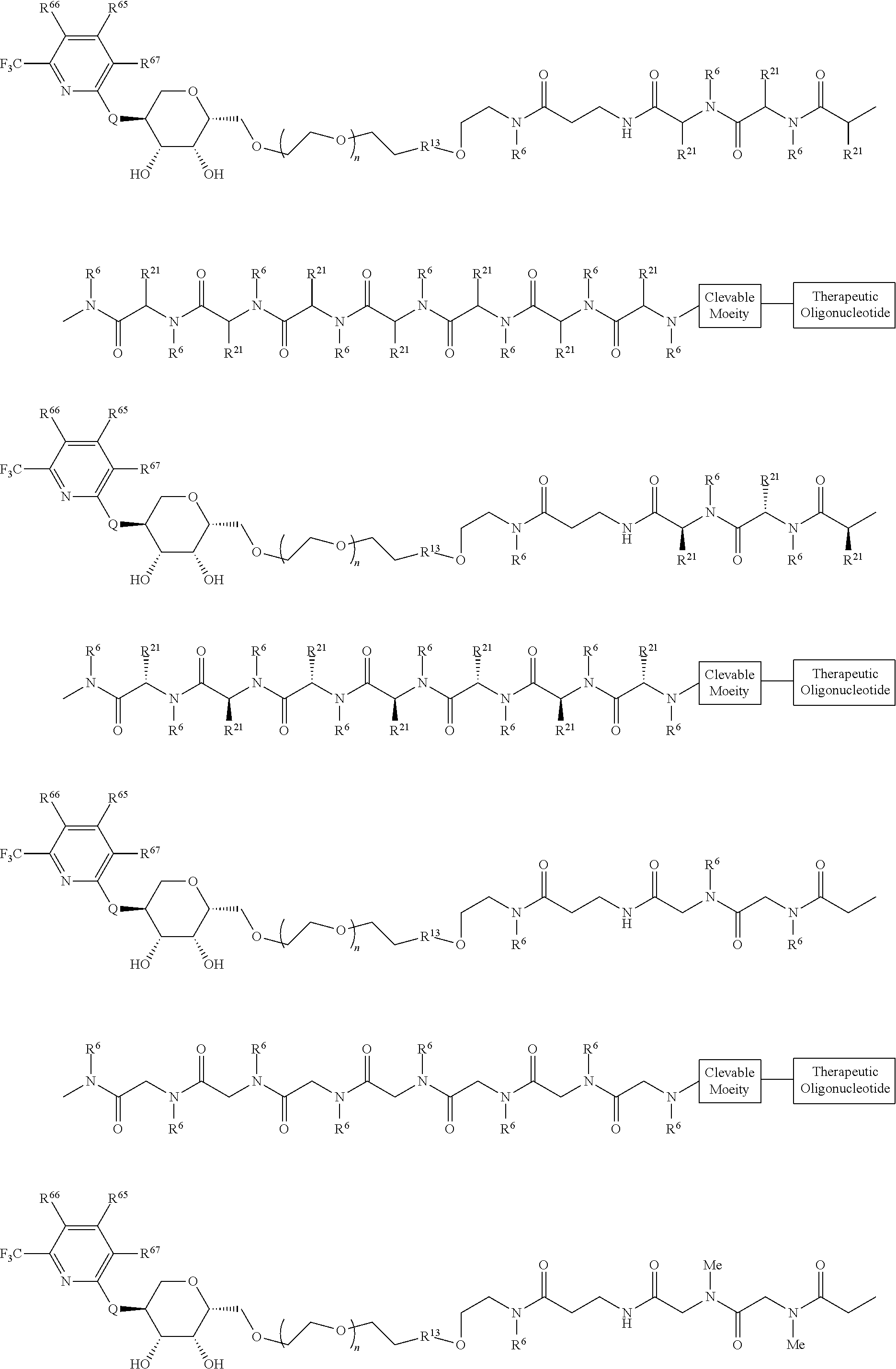

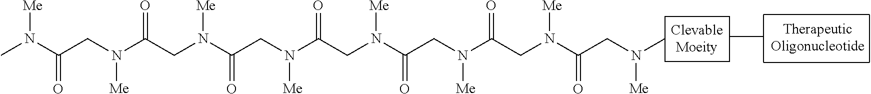

or a pharmaceutically acceptable salt thereof.

In certain embodiments, the compound of the present invention is selected from:

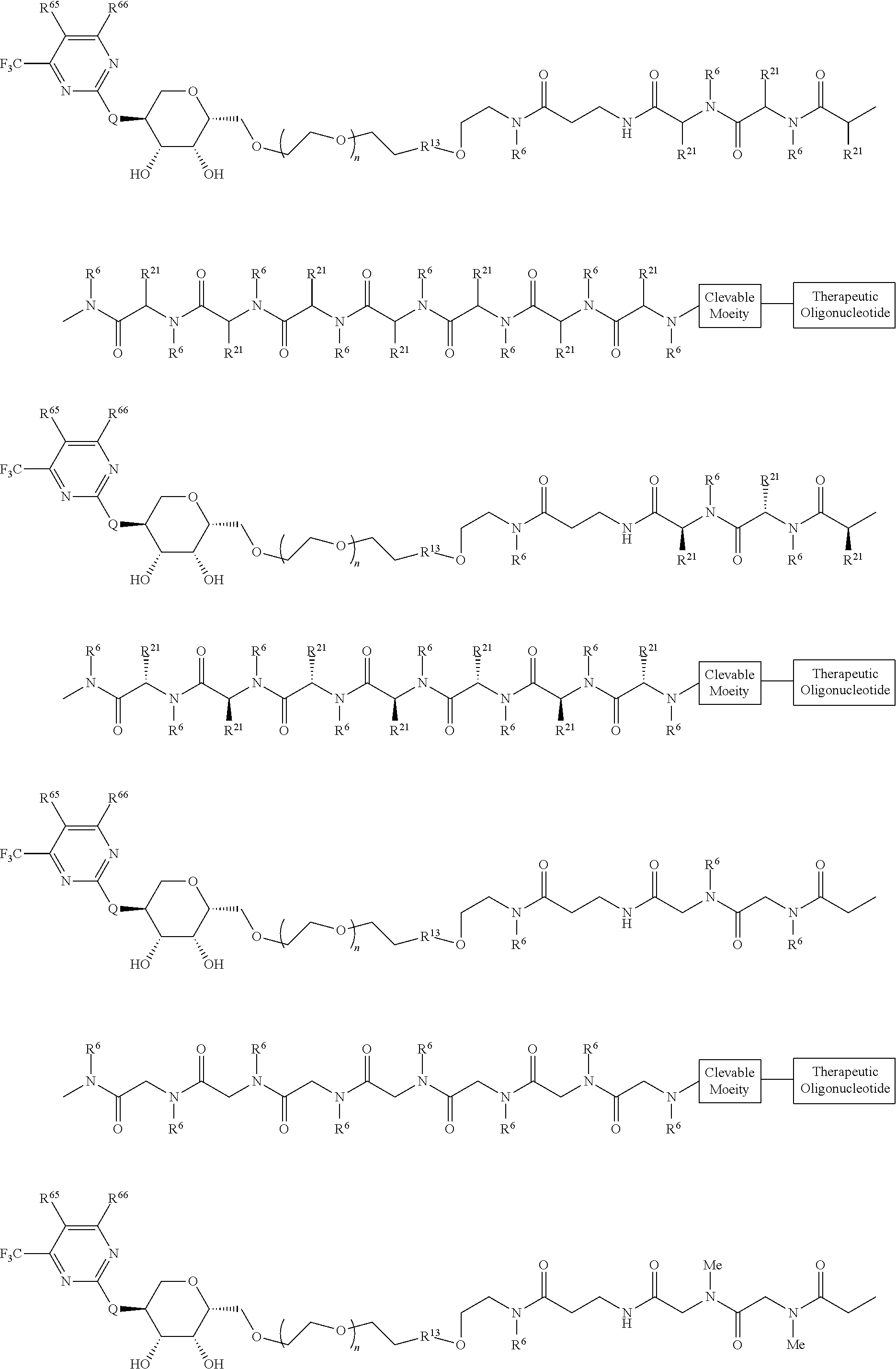

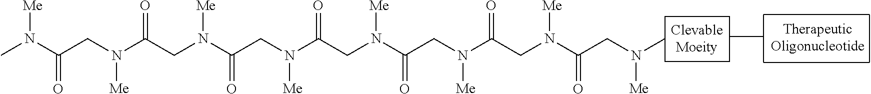

or a pharmaceutically acceptable salt thereof.

In certain embodiments, the compound of the present invention is selected from:

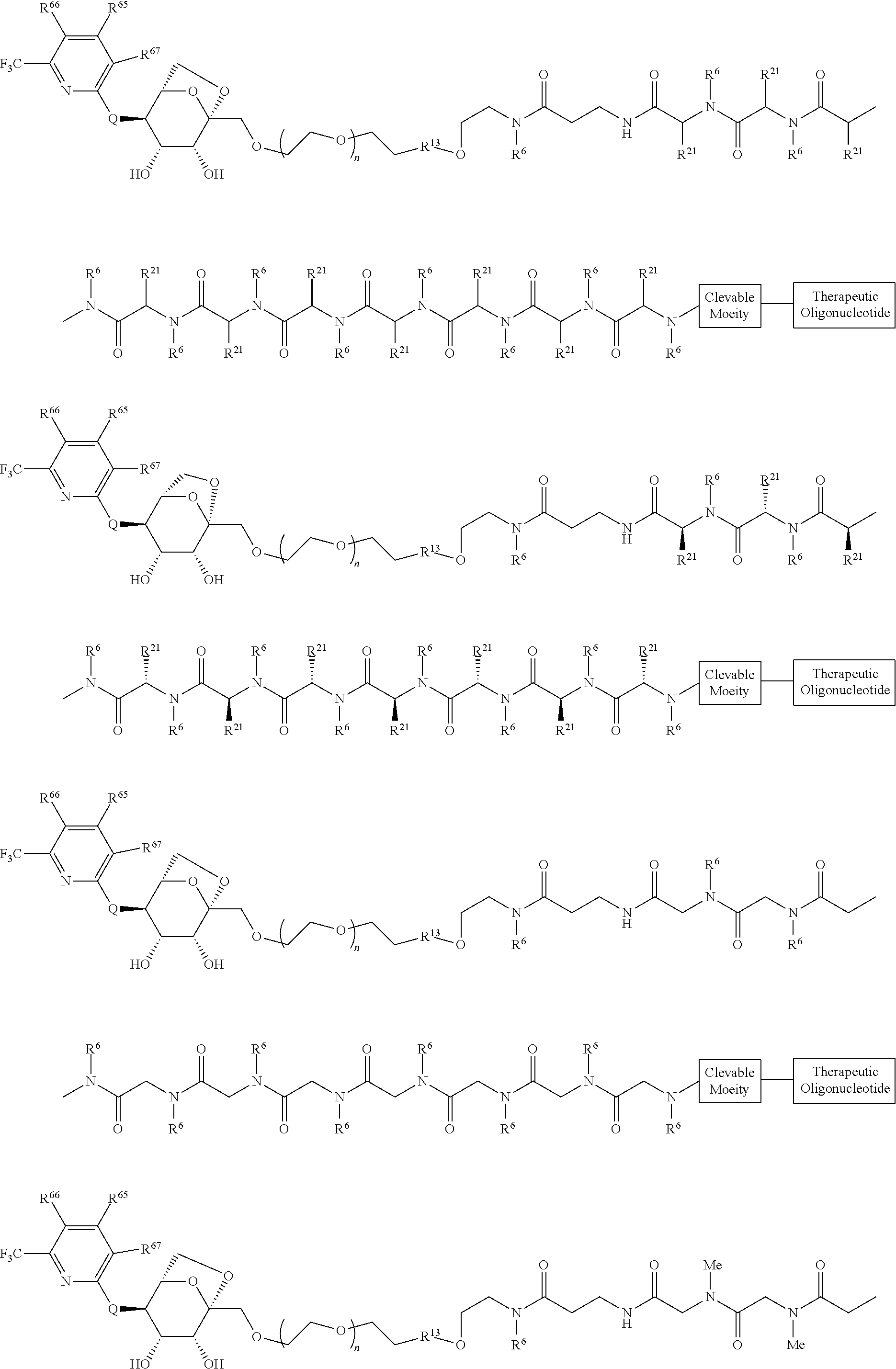

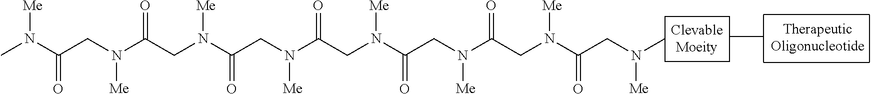

or a pharmaceutically acceptable salt thereof.

In certain embodiments, the compound of the present invention is selected from:

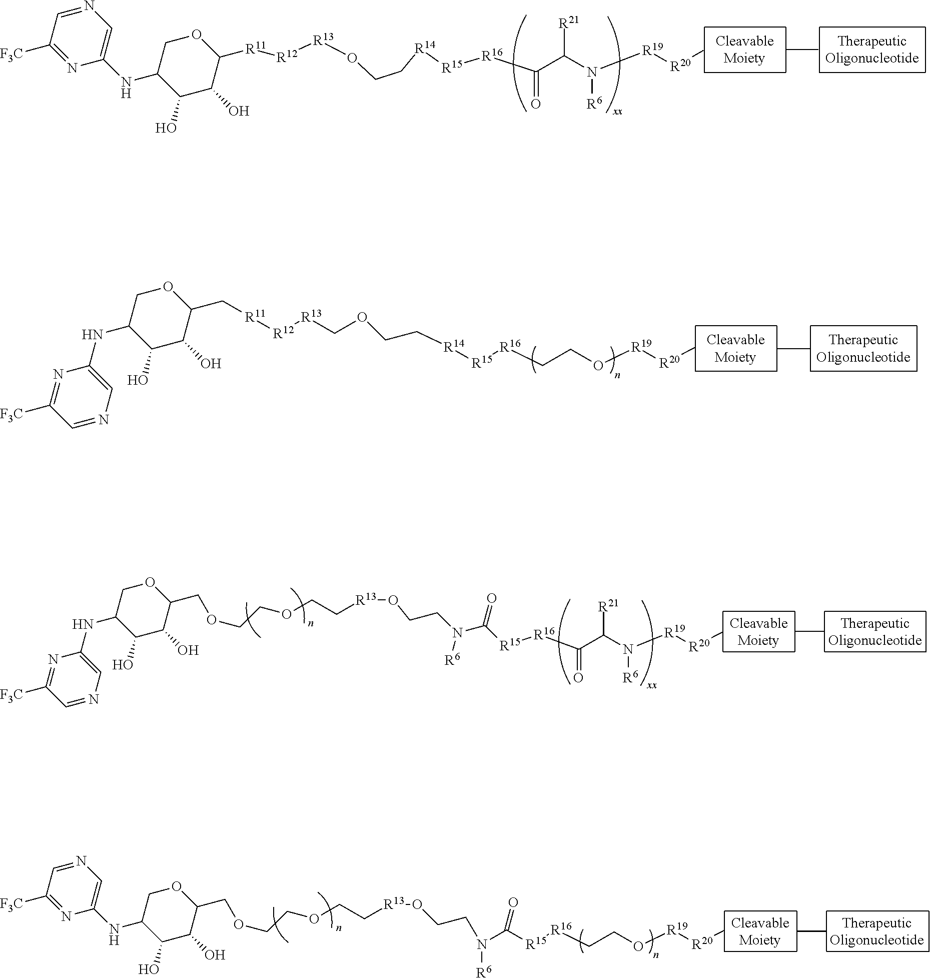

or a pharmaceutically acceptable salt thereof.

In certain embodiments, the compound of the present invention is selected from:

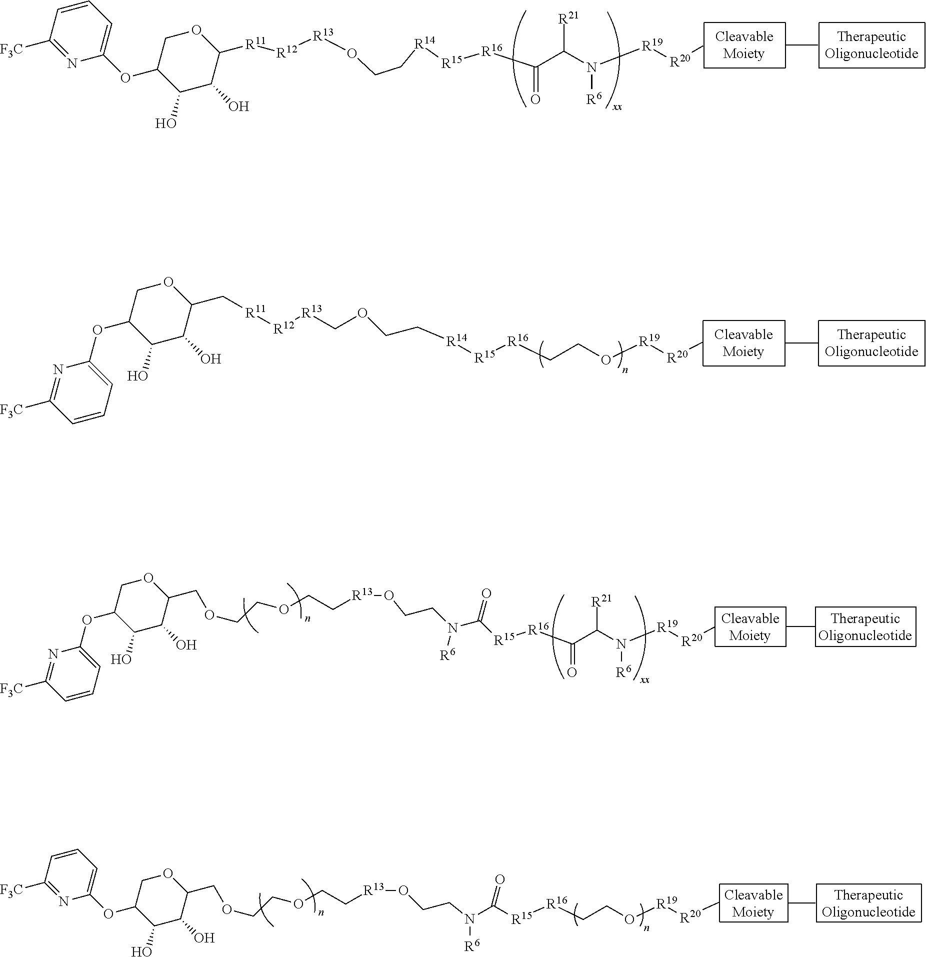

or a pharmaceutically acceptable salt thereof.

In certain embodiments, the compound of the present invention is selected from

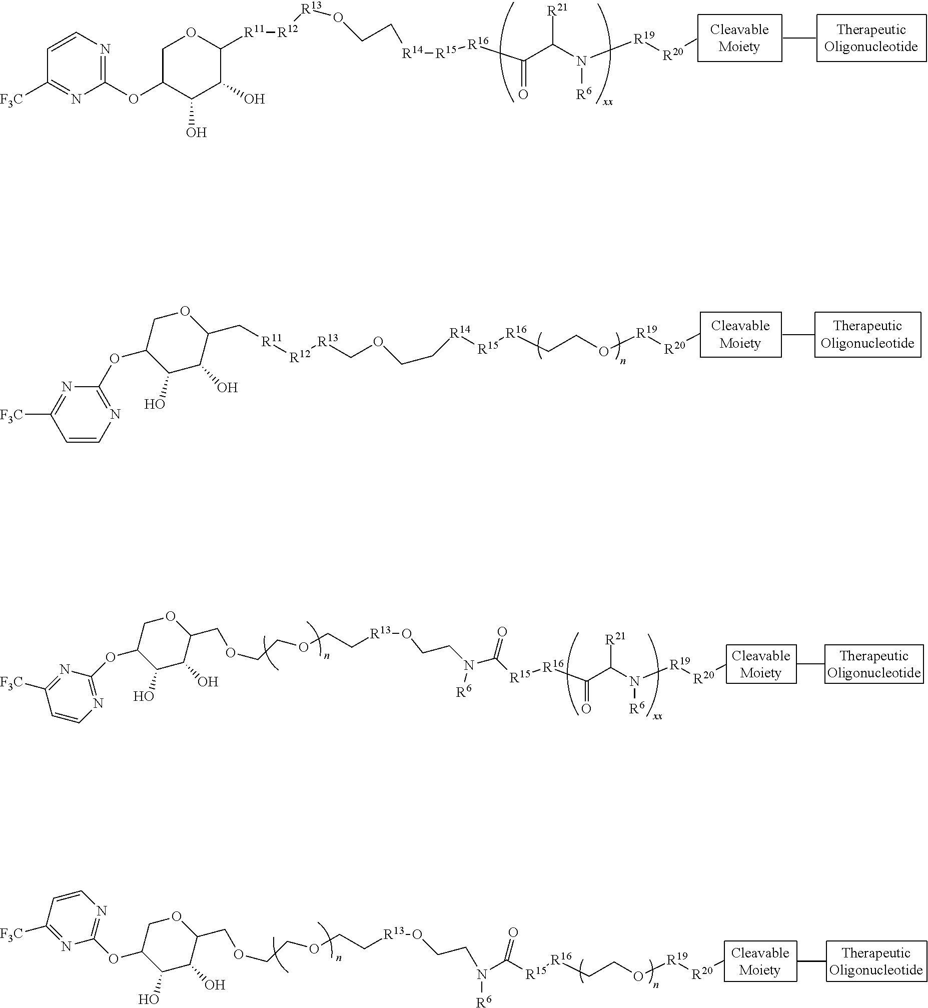

or a pharmaceutically acceptable salt thereof.

In certain embodiments, the compound of the present invention is selected from:

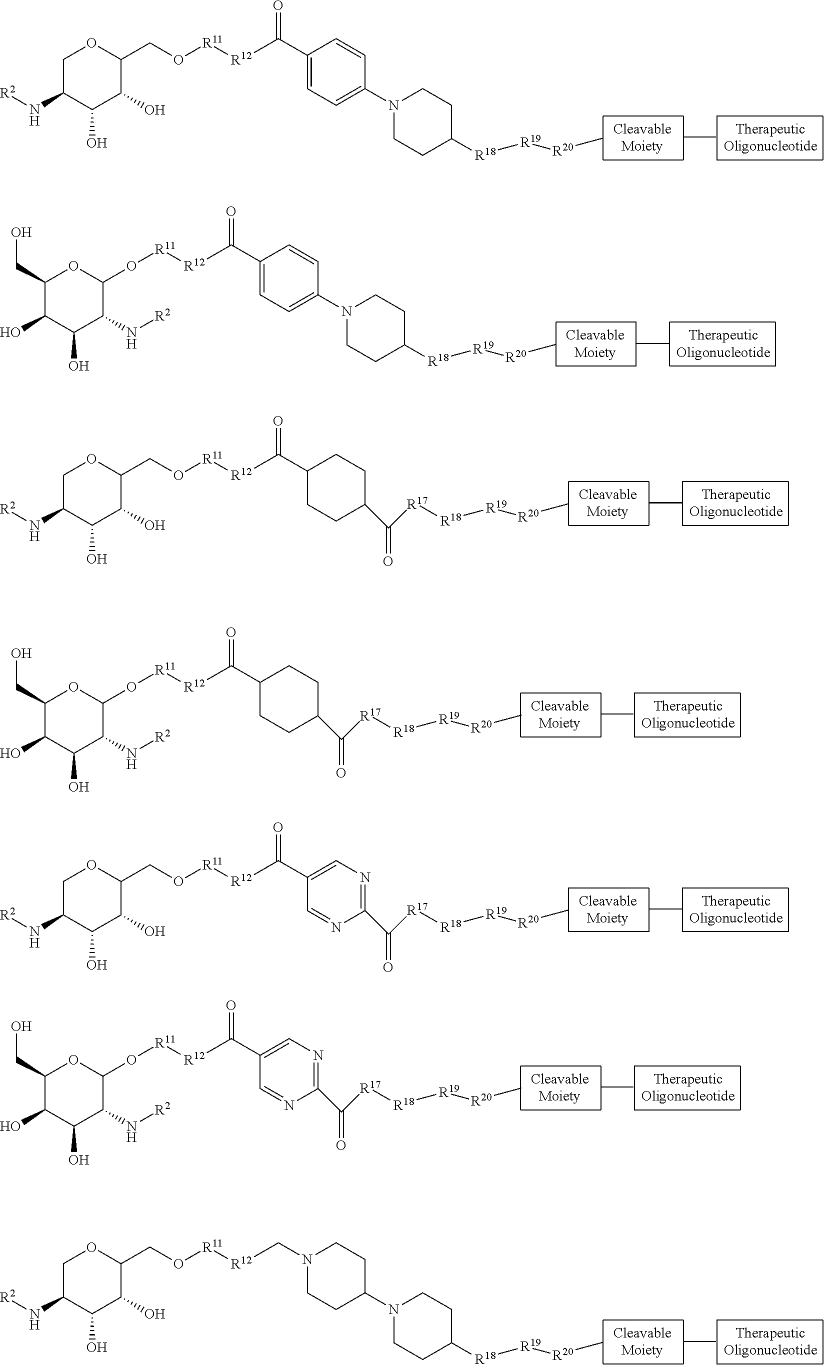

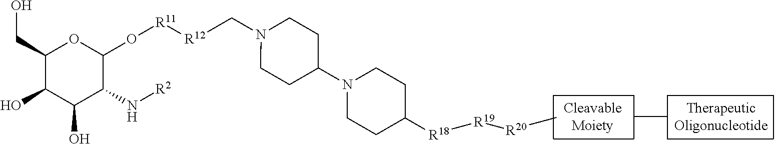

or a pharmaceutically acceptable salt thereof.

In certain embodiments, the compound of the present invention is selected from:

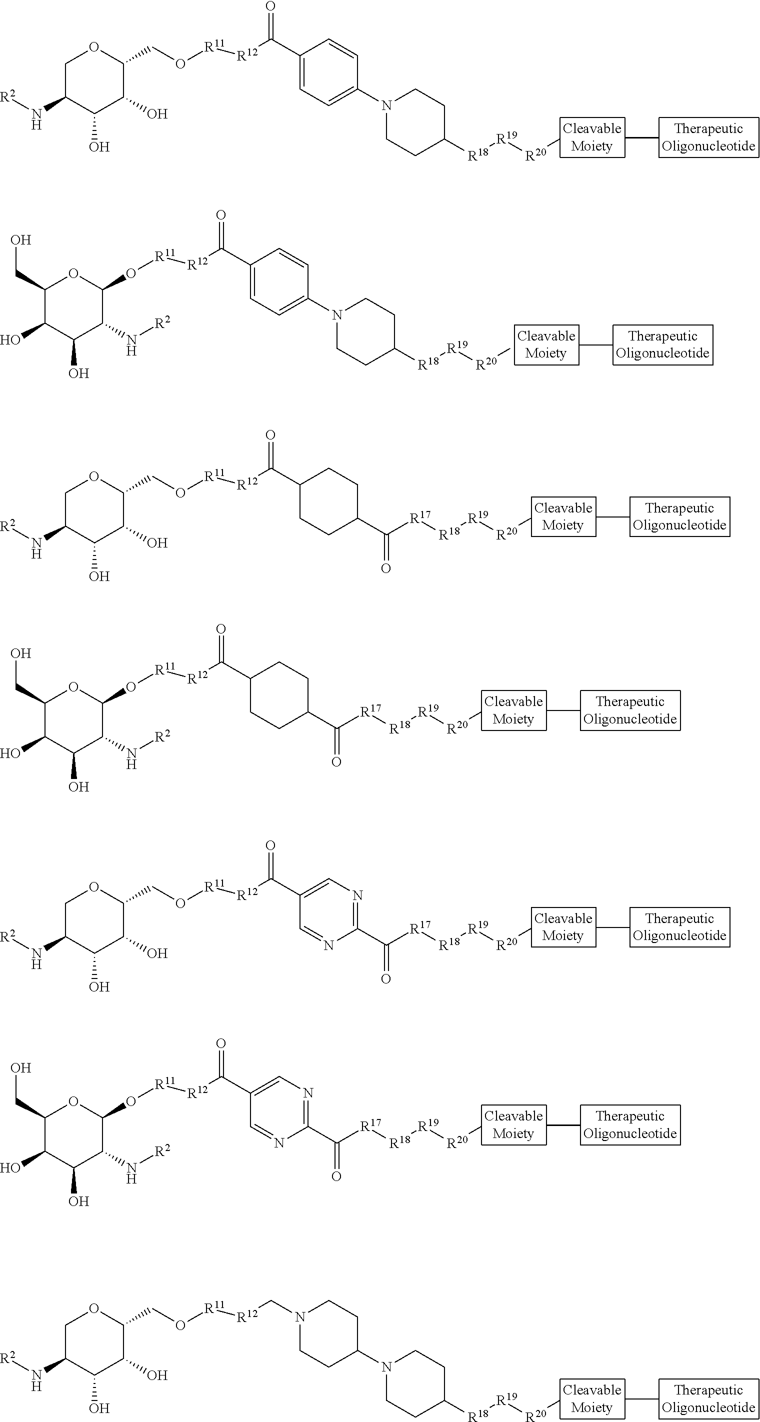

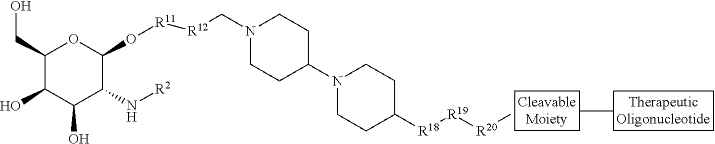

or a pharmaceutically acceptable salt thereof.

In certain embodiments the compound of the present invention is of Formula:

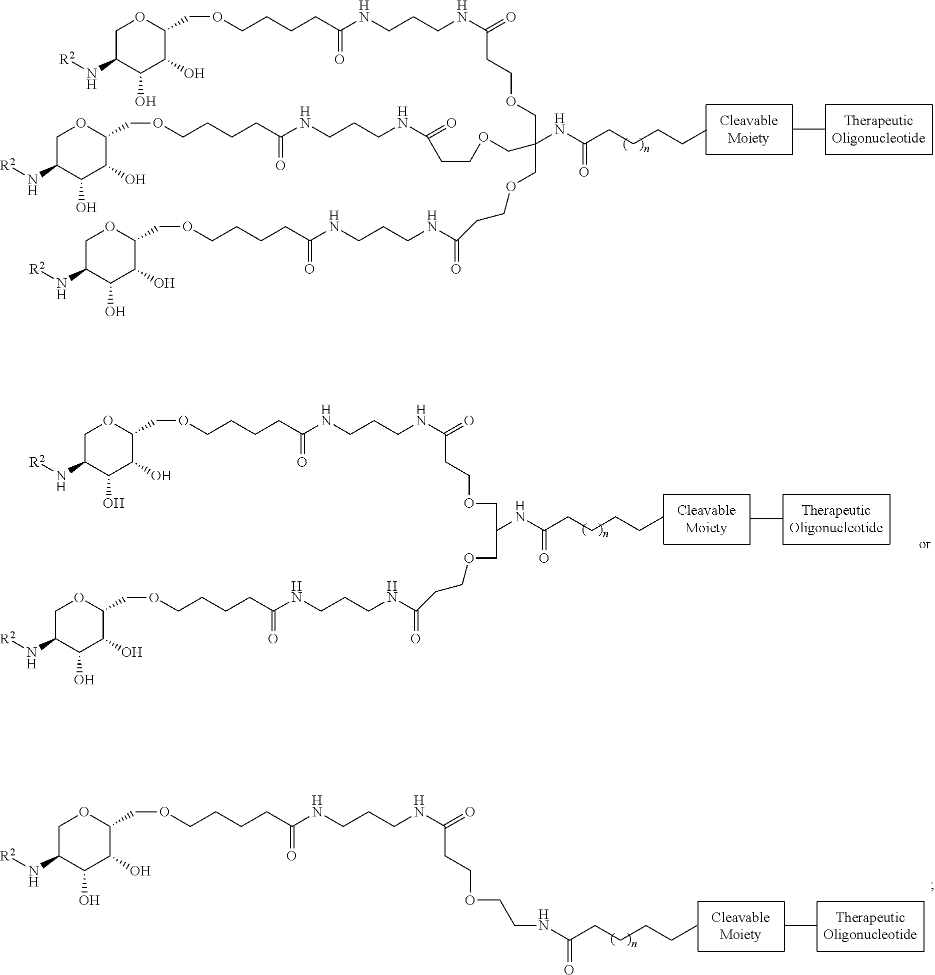

or a pharmaceutically acceptable salt thereof;
wherein
n is 0, 1, 2, 3, 4, 5, 6, 7, 8, 9, or 10; and
R² is

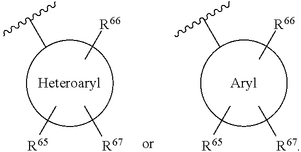

In other embodiments the compound of the present invention is of Formula:

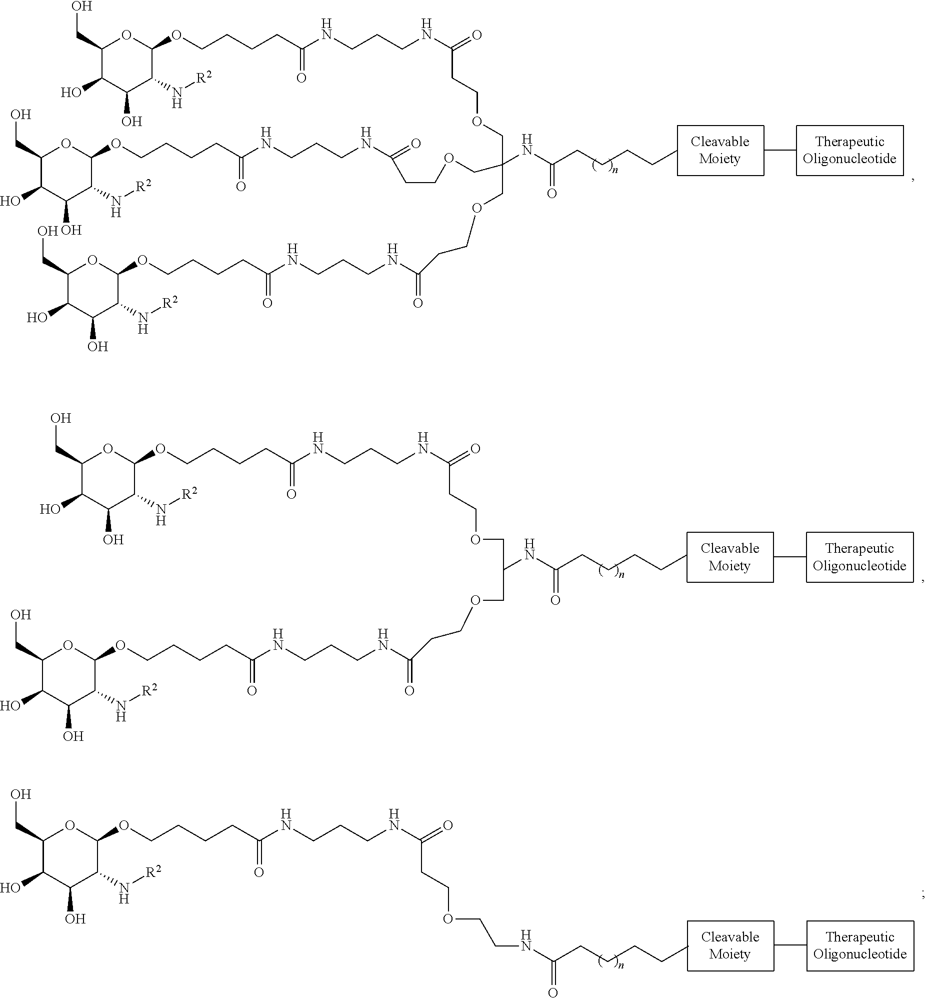

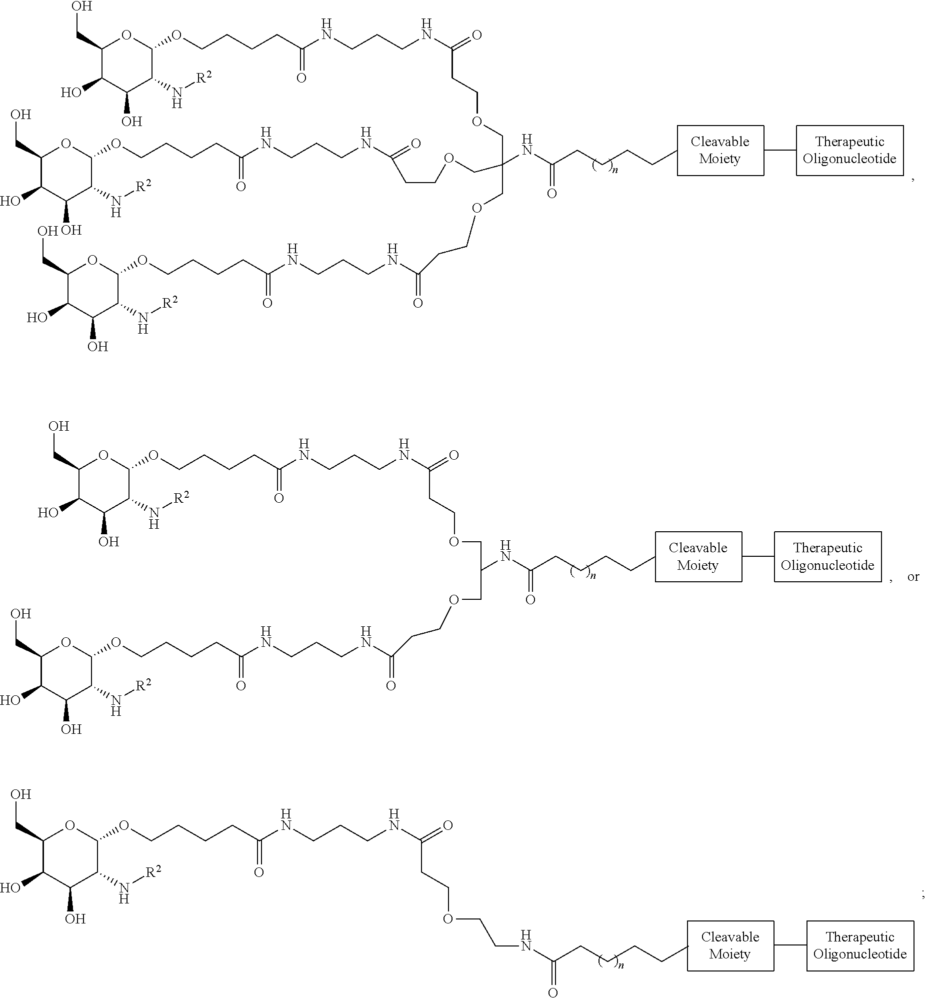

or a pharmaceutically acceptable salt thereof.

In certain embodiments the compound of the present invention is of Formula:

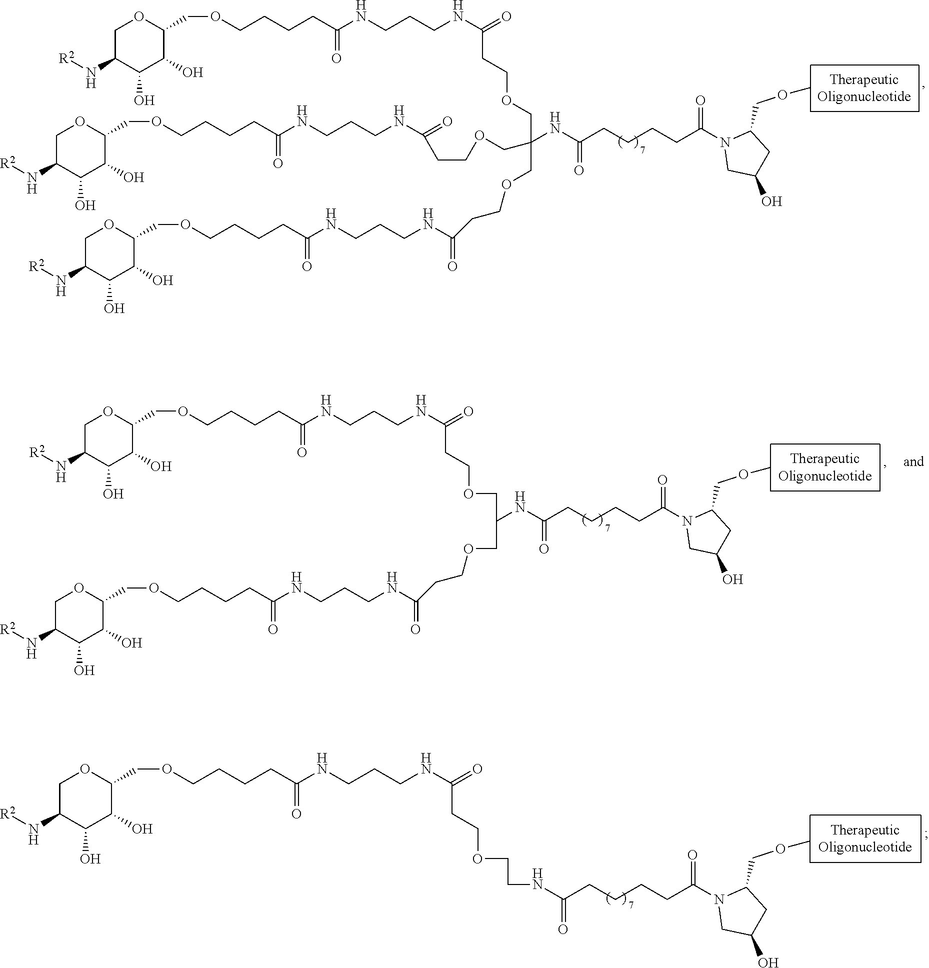

or a pharmaceutically acceptable salt thereof.

In other embodiments the compound of the present invention is of Formula:

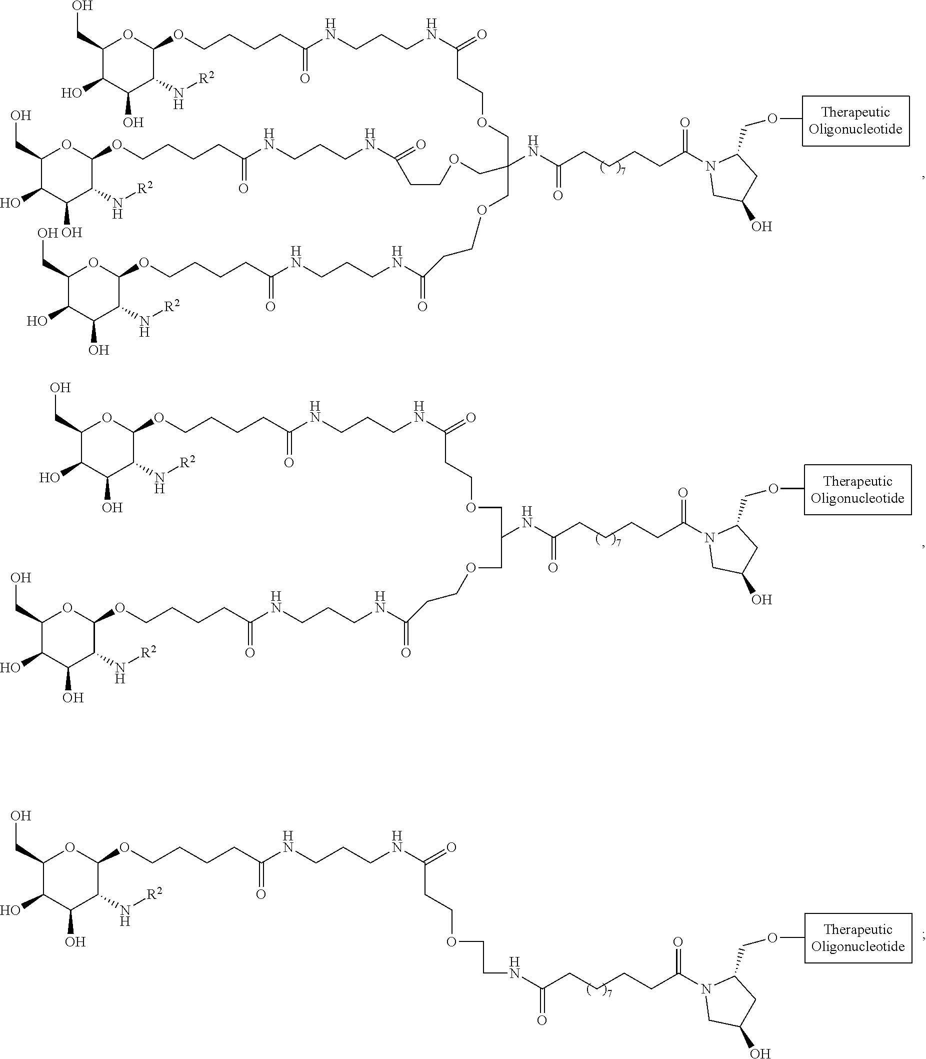

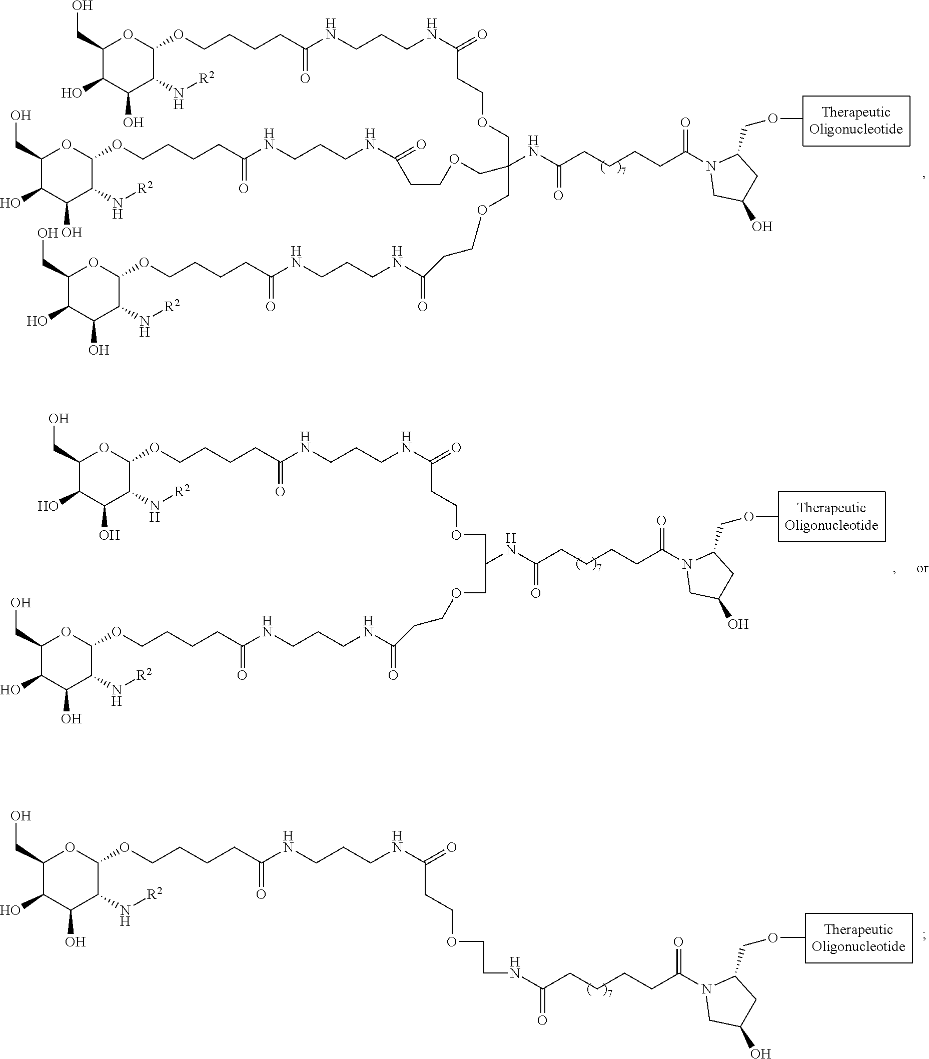

or a pharmaceutically acceptable salt thereof.

In certain embodiments R² is

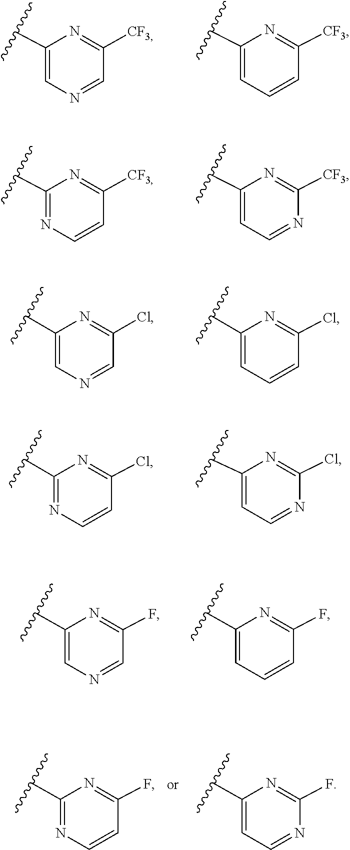

The compounds of the present invention can deliver a Therapeutic Oligonucleotide to the liver. These Therapeutic Oligonucleotide delivering compounds feature select ASGPR ligands that provide high binding affinity for ASGPR. As a result of this high ASGPR binding affinity, these compounds may be administered in lower doses, have fewer side effects, decreased side effects, increased efficacy, faster therapeutic effect, longer metabolic stability, and/or longer therapeutic benefit than conventional Therapeutic Oligonucleotide delivery compounds.

A compound of the present invention can be particularly beneficial in the treatment of a disorder of the liver, such as acute hepatic porphyria. In certain embodiments a compound of the present invention is used to treat primary hyperoxaluria type 1, heterozygous familial hypercholesterolemia, clinical atherosclerotic cardiovascular disease, or hemophilia. In other aspects a compound of the present invention is used to lower the concentration of cholesterol such as low-density lipoprotein cholesterol.

In certain aspects the disorder treated by a compound of the present invention is selected from acute hepatic porphyria (AHP), hyperoxaluria type 1 (PH1), hypercholesterolemia, hemophilia, a complement mediated disorder, HBV, hypertension, nonalcoholic steatohepatitis (NASH), a cardiovascular disease, hyperoxaluria type 2, hyperoxaluria type 3, β-thalassemia, immunoglobulin A nephropathy (IgAN), angioedema, myelodysplastic disorder, and treatment resistant hypertension.

In certain aspects a compound of the present invention is administered prophylactically. For example, when the Therapeutic Oligonucleotide is an antithrombin lowering agent such as fitusiran the compound of the present invention may be administered prophylactically to decrease the risk of excessive bleeding events.

In certain embodiments the compound of the present invention is provided as an isotopically enriched compound, for example a compound with at least one desired isotopic substitution of an atom, at an amount above the natural abundance of the isotope. For example, deuterium can replace one or more hydrogens and ¹³C can replace one or more carbon atoms. In one embodiment, the isotopic substitution is in one or more positions of the ASGPR Binding Ligand. In another embodiment, the isotopic substitution is in one or more positions of the Linker portion of the molecule (Cleavable Moiety, Linker^{A}, Linker^{B}, Linker^{C}, or Linker^{D}). In another embodiment, the isotopic substitution is in one or more positions of the Therapeutic Oligonucleotide portion of the molecule.

The present invention thus includes at least the following features:
(i) A Therapeutic Oligonucleotide delivering compound described herein or a pharmaceutically acceptable salt thereof;
(ii) Use of a Therapeutic Oligonucleotide delivering compound described herein or a pharmaceutically acceptable salt thereof, in treating a medical disorder, for example a disorder of the liver;
(iii) An isotopically enriched derivative of a Therapeutic Oligonucleotide delivering compound described herein or a pharmaceutically acceptable salt thereof;
(iv) A process for manufacturing a medicament intended for the therapeutic use for treating or preventing a disorder, characterized in that a Therapeutic Oligonucleotide delivering compound described herein or a pharmaceutically acceptable salt thereof is used in the manufacture;
(v) A Therapeutic Oligonucleotide delivering compound described herein or a pharmaceutically acceptable salt thereof in a purified or substantially pure form (e.g., at least 90, 95, 96, 97, 98, 99, 99.5, or 99.9%);
(vi) A Therapeutic Oligonucleotide delivering compound described herein or a pharmaceutically acceptable salt thereof to treat a disorder described herein; and
(vii) A method for the manufacture of a Therapeutic Oligonucleotide delivering compound described herein.

### BRIEF DESCRIPTION OF THE FIGURES

FIG. 1A, FIG. 1B, FIG. 1C, and FIG. 1D are dose-response curves showing siRNA silencing of the target gene in a primary human hepatocyte assay (Example 7). Two exemplary Therapeutic Oligonucleotide delivering compounds were tested against the compound Givosiran. After 48 hours of incubation, both Therapeutic Oligonucleotide delivering compounds silenced the target gene more potently than Givosiran.

FIG. 2A and FIG. 2B are time course plots of test articles (Compound 1 and Givosiran) at two different concentrations 2.3 nM and 9.4 nM respectively. At both concentrations, Compound 1 silenced the target gene more potently than Givosiran at each time point (16 hours, 24 hours, 48 hours, and 72 hours). The experiment was conducted according to the procedure of Example 7.

FIG. 3 is a time course plot of Compound 1 concentration in Rat plasma. It describes pharmacokinetics of Compound 1 (5 mg/kg dose) in Rat for IV bolus and SC administration. Initial plasma concentration of Compound 1 in IV dosed rats was detected to be >10 μg/mL and drops to about 100 ng/mL within 0.5 hour. Initial plasma concentration of Compound 1 in SC dosed rats was detected to be >100 ng/mL and drops to about 100 ng/mL at 4 hours. The experiment was conducted according to the procedure of Example 8.

FIG. 4 is a boxplot of dose dependent liver exposure for Compound 1 and Givosiran at 1 mg/kg, 2.5 mg/kg, 5 mg/kg, and 10 mg/kg dosages in rats at 72 hours post administration. For each dosage Compound 1 shows lower liver concentration than Givosiran at 72 hours. At 10 mg/kg dosage, Givosiran concentration in liver was about 28 μg/g, while Compound 1 concentration in liver was about half (~14 μg/g). The experiment was conducted according to the procedure of Example 9.

FIG. 5A and FIG. 5B show ALAS1 mRNA suppression at 72 hours, where rats were dosed SC with vehicle, Givosiran or Compound 1 at 1, 2.5, 5 or 10 mg/kg and livers were harvested for target mRNA analysis 72 hours later. FIG. 5A is a dose response curve showing Mean+SD of % knock down for 5 rats per dose group. FIG. 5B is a boxplot showing the dose response area under the curve (AUC). Unpaired t test with Welch's correction (SD not assumed equal) were performed at each dose group between Givosiran and Compound 1, *p<0.05. The experiment was conducted according to the procedure of Example 9.

### DETAILED DESCRIPTION OF THE INVENTION

Compounds and their pharmaceutically acceptable salts and compositions thereof that deliver a Therapeutic Oligonucleotide, as well as starting materials and intermediates for such Therapeutic Oligonucleotide delivering compounds and their methods of use and manufacture are provided. These Therapeutic Oligonucleotide delivering compounds are highly potent binders of ASGPR and include a Cleavable Moiety and a Therapeutic Oligonucleotide. In certain embodiments the Therapeutic Oligonucleotide delivering compound binds to ASGPR and is then trafficked into the cell where the Cleavable Moiety is cleaved and the Therapeutic Oligonucleotide is released. In some embodiments, a Therapeutic Oligonucleotide delivering compound that incorporates one of the high binding ASGPR ligands as described herein can be sufficiently active in the form of a monodentate compound (i.e., 1:1 ratio of Therapeutic Oligonucleotide to ASGPR ligand in the therapeutic molecule).

In certain embodiments, the Therapeutic Oligonucleotide delivering compound selectively delivers the therapeutic nucleotide to the liver.

### I. Definitions

Compounds are described using standard nomenclature. Unless defined otherwise, all technical and scientific terms used herein have the same meaning as is commonly understood by one of skill in the art to which this invention belongs.

The compounds described herein include independently the enantiomers, mixtures of enantiomers, diastereomers, tautomers, racemates and other isomers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes compounds with at least one desired isotopic substitution of an atom, at an amount above the natural abundance of the isotope, i.e., enriched.

Examples of isotopes that can be incorporated into compounds, of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F, ³¹P, ³²P, ³⁵S, ³⁶Cl, and ¹²⁵I respectively. In one embodiment, isotopically labeled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically labeled compounds of this invention and prodrugs thereof can generally be prepared by carrying out the procedures disclosed in the schemes or in the examples and preparations described below by using a readily available isotopically labeled reagent instead of a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C₀, may be used. In one embodiment, the isotopic substitution is accomplished by replacing hydrogen with a deuterium at one or more locations on the molecule to improve the performance of the drug, for example, the pharmacodynamics, pharmacokinetics, biodistribution, half-life, stability, AUC, Tₘₐₓ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, is typically partial. Partial isotopic substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in certain nonlimiting embodiments is enough to alter a detectable property of the drug in a human.

A “dosage form” means a unit of administration of an active agent. Examples of dosage forms include intravenous formulations, solid dosage forms, tablets, capsules, injections, suspensions, liquids, emulsions, implants, particles, spheres, creams, ointments, suppositories, inhalable forms, transdermal forms, buccal, sublingual, parenteral, systemic, topical, gel, mucosal, implant, and the like.

“Pharmaceutical compositions” are compositions comprising at least one active agent, and at least one other pharmaceutically acceptable substance, such as a carrier. The present invention includes pharmaceutical compositions of the Therapeutic Oligonucleotide delivering compounds.

“Pharmaceutical combinations” are combinations of at least two active agents which may be combined in a single dosage form or provided together in separate dosage forms.

A “pharmaceutically acceptable salt” is a derivative of the disclosed compound wherein the parent molecule is modified by making inorganic and organic, pharmaceutically acceptable, acid or base addition salts thereof. The salt of a Therapeutic Oligonucleotide delivering compound can be synthesized by reaction of a basic or acidic moiety by conventional chemical methods. Generally, such salts can be prepared by reacting free acid forms of a Therapeutic Oligonucleotide delivering compound with a stoichiometric amount of the appropriate base (such as Na, Ca, Mg, or K hydroxide, carbonate, bicarbonate, or the like), or by reacting free base forms of Therapeutic Oligonucleotide delivering compound with a stoichiometric amount of the appropriate acid. Such reactions are typically carried out in water or in an organic solvent, or in a mixture of the two.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using an acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a pharmaceutically acceptable diluent, excipient, or vehicle with which an active compound is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In one embodiment, an excipient is used that is acceptable for veterinary use.

A “patient” or “host” or “subject” is a human or non-human animal in need of treatment or prevention of any of the disorders as specifically described herein. Typically, the host, patient, or subject is a human. A “patient” or “host” or “subject” may in certain embodiments where warranted include a veterinary application, for example, to a mammal, primate (e.g., human), horse, dog, cat, cow, sheep, goat, and the like.

A “therapeutically effective amount” of a Therapeutic Oligonucleotide delivering compound, pharmaceutical composition, or combination of this invention means an amount that when administered to a host provides a therapeutic benefit such as an amelioration of symptoms or reduction or diminution of the disease itself.

In one embodiment, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a “′” or an “a” designation, which in one embodiment can be deuterated.

The term “immunoglobulin,” typically refers to a large Y-shaped protein (e.g. an antibody) that identifies and neutralizes a foreign compound or object such as a pathogen or disease tissue. Non-limiting examples of immunoglobulin proteins include IgA, IgD, IgE, IgG, and IgM. An immunoglobulin as used herein may also include a binding fragment as known to the skilled worker.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric.

A dash (“-”) that is not between two letters or symbols is used to indicate a point of attachment for a substituent. For example, —(C═O)NH₂ is attached through carbon of the keto (C═O) group.

The term “percent (%) identity”, “sequence identity”, “percent sequence identity”, or “percent identical” in the context of nucleic acid sequences or amino acid sequences refers to the residues in the two sequences which are the same when aligned for correspondence. Unless otherwise specified, it is to be understood that a percentage of identity is a minimum level of identity and encompasses all higher levels of identity up to 100% identity to the reference sequence. For example, “95% identity” and “at least 95% identity” may be used interchangeably and include 95, 96, 97, 98, 99 up to 100% identity to the referenced sequence, and all fractions therebetween. The length of sequence identity comparison may be over the full-length of the genome, the full-length of a gene coding sequence, a fragment of a gene coding sequence, a fragment of at least about 500 to about 5000 nucleotides, the full-length of an amino acid sequence, a fragment of an amino acid sequence, a fragment of at least about 8 to about 200 amino acids, is desired. Generally, when referring to “identity”, “homology”, or “similarity” between two different sequences, “identity”, “homology” or “similarity” is determined in reference to “aligned” sequences. “Aligned” sequences or “alignments” refer to more than one nucleic acid sequences or amino acid sequences, often containing corrections for missing or additional bases or amino acids as compared to a reference sequence. Multiple sequence alignment programs are also available for nucleic acid sequences and amino acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those skilled in the art.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridone. Combinations of substituents and/or variables are permissible only if such combinations result in stable compounds or useful synthetic intermediates.

“Alkyl” is a branched, straight chain, or cyclic saturated aliphatic hydrocarbon group. Unless denoted otherwise, “alkyl” is typically C₁-C₁₂ alkyl. In certain embodiments the alkyl contains from 1 to about 12 carbon atoms, more generally from 1 to about 6 carbon atoms, from 1 to about 4 carbon atoms, or from 1 to 3 carbon atoms. In one embodiment, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example, and without limitation, the terms alkyl, alkenyl, alkynyl, alkoxy, alkanoyl, alkenloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Unless denoted otherwise, “alkenyl” is typically C₂-C₁₂ alkenyl. Nonlimiting examples are C₂-C₈alkenyl, C₂-C₇alkenyl, C₂-C₈alkenyl, C₂-C₈alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain. Unless denoted otherwise, “alkynyl” is typically C₂-C₁₂ alkynyl. In certain embodiments alkynyl is C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

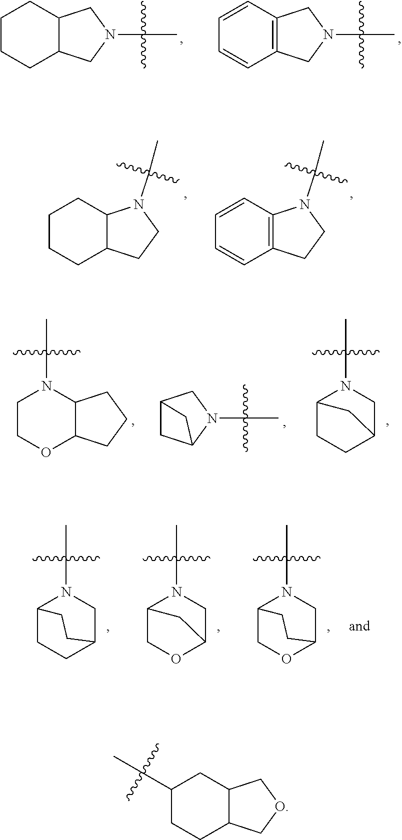

Unless otherwise drawn or clear from the context, the term “bicyclic heterocycle” includes cis and trans diastereomers. Non-limiting examples of chiral bicyclic heterocycles include

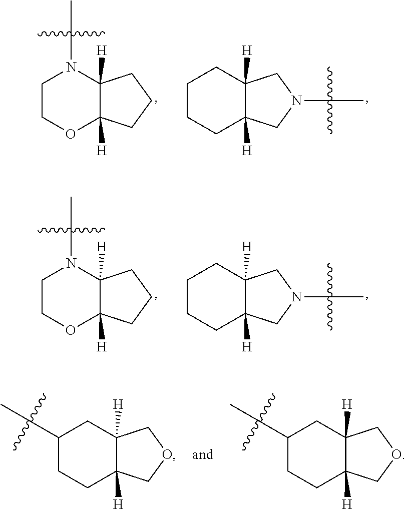

In certain alternative embodiments the term “heterocycle” refers to saturated and partially saturated heteroatom-containing ring radicals, where the heteroatoms may be selected from N, S, O, B, Si, and P.

“Heteroaryl” refers to a stable monocyclic, bicyclic, or multicyclic aromatic ring which contains from 1 to 3, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein.

“Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

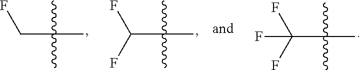

Additional non-limiting examples of “haloalkyl” include:

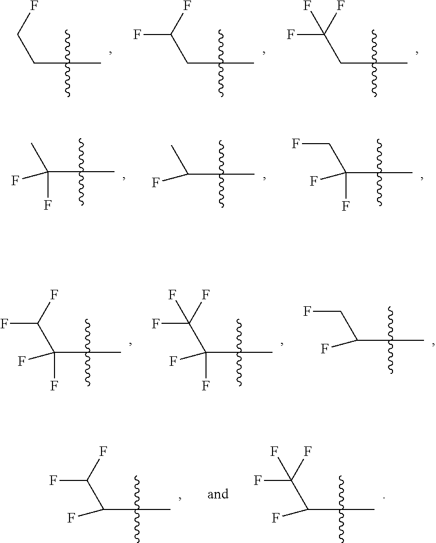

Additional non-limiting examples of “haloalkyl” include:

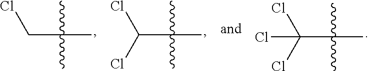

Additional non-limiting examples of “haloalkyl” include:

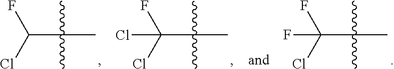

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

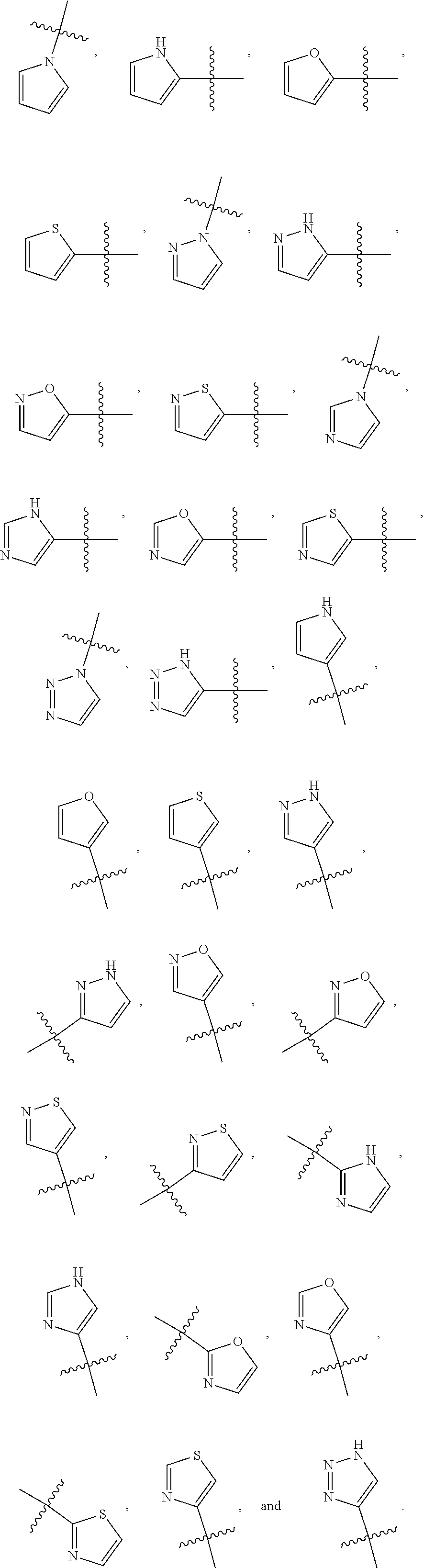

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

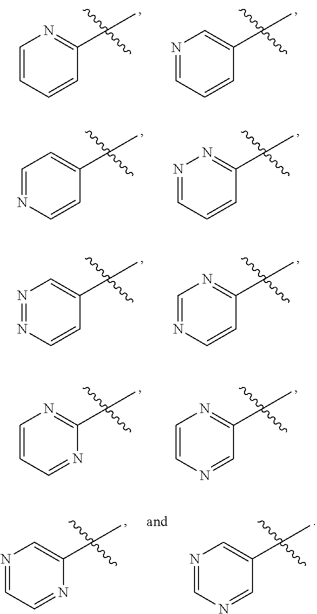

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

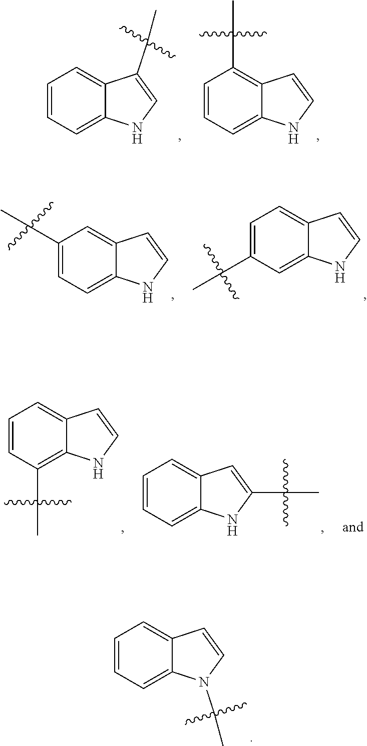

Additional non-limiting examples of “heteroaryl” groups that are bicyclic include:

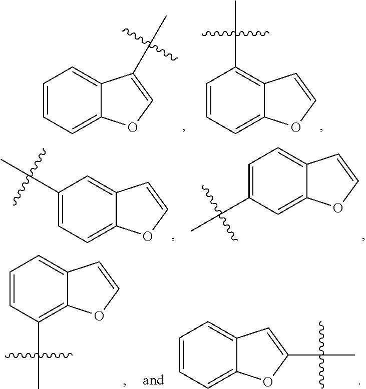

Additional non-limiting examples of “heteroaryl” groups that are bicyclic include:

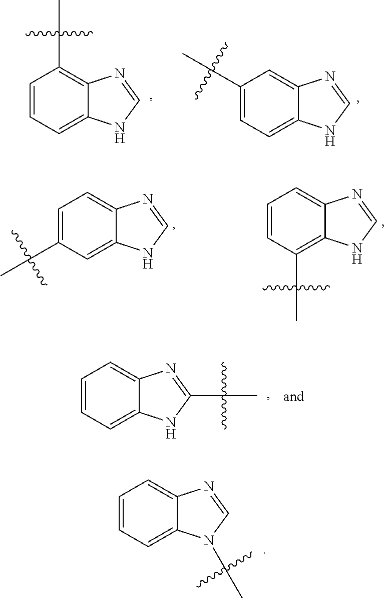

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

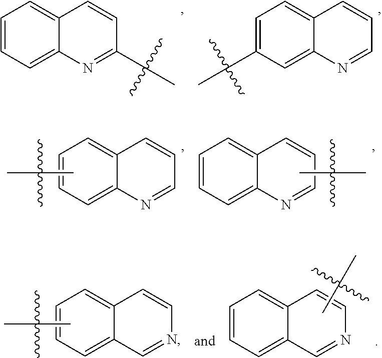

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

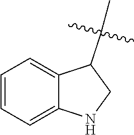 [SMILES: *C1CNc2ccccc21]

is a “heterocycle” group.

However,

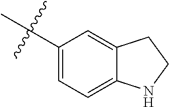 [SMILES: *c1ccc2c(c1)CCN2]

is an “aryl” group.

Non-limiting examples of “heterocycle” also include:

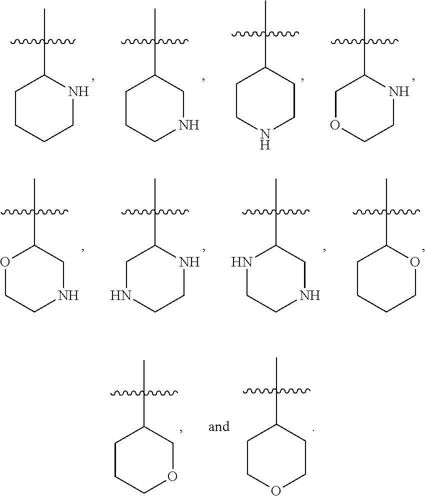

Additional non-limiting examples of “heterocycle” include:

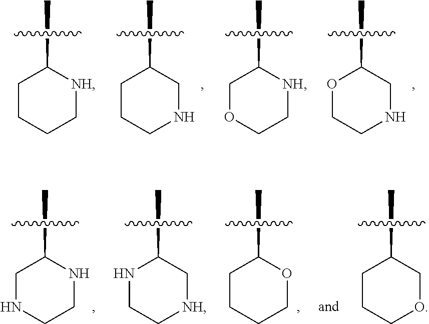

Additional non-limiting examples of “heterocycle” include:

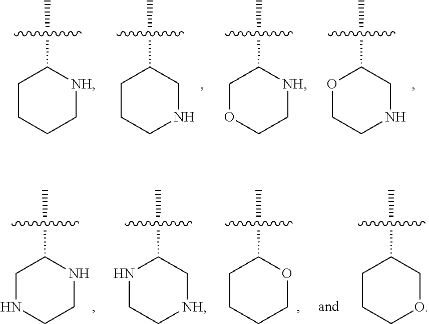

Non-limiting examples of “heterocycle” also include:

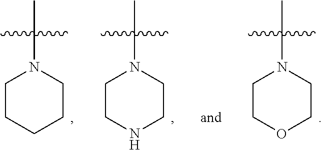

Non-limiting examples of “heterocycle” also include:

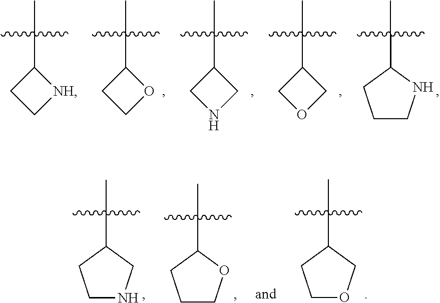

Additional non-limiting examples of “heterocycle” include:

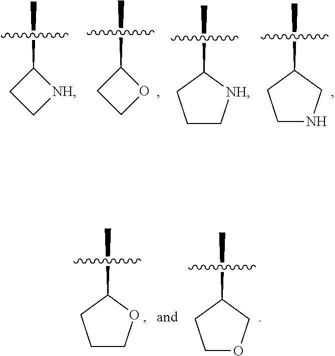

Additional non-limiting examples of “heterocycle” include:

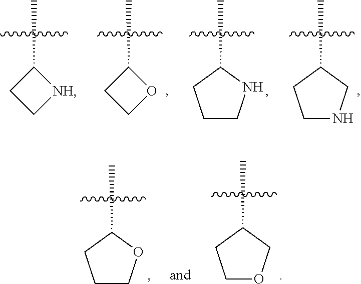

### Embodiments of “Aryl”

In certain embodiments “aryl” is a 6 carbon aromatic group (phenyl).

In certain embodiments “aryl” is a 10 carbon aromatic group (naphthyl).

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

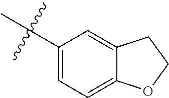 [SMILES: *c1ccc2c(c1)CCO2]

an “aryl” group.

However,

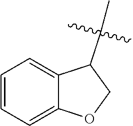 [SMILES: *C1COc2ccccc21]

is a “heterocycle” group.

### Embodiments of “Arylalkyl”

Non-limiting examples of “arylalkyl” include:

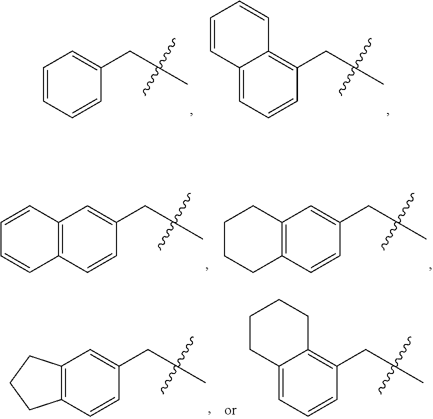

In certain embodiments “arylalkyl” is

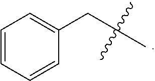 [SMILES: *Cc1ccccc1]

In certain embodiments the “arylalkyl” refers to a 2 carbon alkyl group substituted with an aryl group.

Non-limiting examples of “arylalkyl” include:

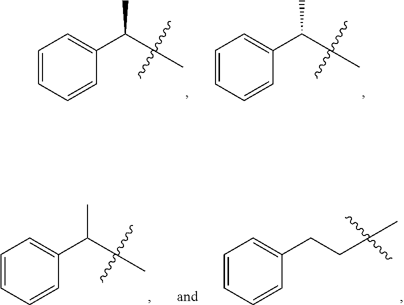

### II. Therapeutic Oligonucleotide

In certain embodiments, the Therapeutic Oligonucleotide is an antisense oligonucleotide. Antisense oligonucleotides are composed of linked nucleosides, wherein each nucleoside has a sugar moiety and a nucleobase. Nucleosides are linked together through internucleoside linkages (e.g. phosphates) to form an oligonucleotide. The structure of an antisense oligonucleotide may be considered in terms of chemical features (e.g., modifications and patterns of modifications) and nucleobase sequence (e.g., sequence of antisense oligonucleotide, identity and sequence of target nucleic acid).

In certain embodiments, the Therapeutic Oligonucleotide is a DNA fragment. A DNA fragment is made up of linked nucleosides, each nucleoside comprising a sugar moiety and a nucleobase. The structure of a DNA fragment may be considered in terms of chemical features (e.g., modifications and patterns of modifications) and nucleobase sequence (e.g., sequence of oligonucleotides or identity).

In certain embodiments, the Therapeutic Oligonucleotide is an siRNA. An siRNA Therapeutic Nucleotide can have linked nucleosides, each nucleoside comprising a sugar moiety and a nucleobase. The siRNA can include modifications that stabilize the oligonucleotide, either stabilize or derivatize the nucleobase sequence or make conservative or non-conservative nucleotide substitutions.

In certain embodiments, the Therapeutic Oligonucleotide contains a nucleoside which is a modified form of a natural or nonnatural nucleoside. In certain embodiments, the Therapeutic Oligonucleotide has one or more modified nucleosides and/or modified internucleoside linkages. In certain embodiments, a modified nucleoside is modified at the sugar moiety and/or the nucleobase.

### Modified Sugar Moieties

In certain embodiments, one or more of the nucleosides in a Therapeutic Oligonucleotide has a modified sugar moiety. A Therapeutic Oligonucleotide with one or more modified sugar moieties may have desirable properties, such as enhanced nuclease stability. The one or more modified sugar moieties can also increase the binding affinity of the Therapeutic Oligonucleotide for its target nucleic acid sequence. In certain embodiments, a modified sugar moiety is a substituted sugar moiety. In certain embodiments, a modified sugar moiety is a sugar surrogates. A sugar surrogate is a nonnatural molecule which can mimic one or more functions of the nucleoside sugar moiety. For example, the nucleoside reverse-transcriptase inhibitor emtricitabine is a nucleoside which has a 1,3 oxothiolane sugar surrogate.

In certain embodiments, a modified sugar moiety is a substituted sugar moiety comprising one or more non-bridging sugar substituents, including but not limited to substituents at the 2′ and/or 5′ positions. Where a substituent introduces chirality, the modified nucleoside can be in any desired ratio of R- and S-enantiomers, including a racemic mixture and pure enantiomers. In some embodiments, the modified nucleoside can be at least 90% free of the opposite enantiomer. In some embodiments, the modified nucleoside can be at least about 75%, 80%, 85%, 90%, 95%, 98%, 99%, or even 100% free of the opposite enantiomer. Where multiple substituents form diastereomers, the modified nucleoside can be in any desired ratio of diastereomers (for example, cis and trans diastereomers). In certain embodiments, the modified nucleoside is diastereopure. In certain embodiments, the modified nucleoside is a 1:1 mixture of diastereomers. In certain embodiments, the modified nucleoside is at least 75%, 80%, 85%, 90%, 95%, 98%, 99% or even 100% diastereopure.

In certain embodiments, the modified sugar moiety is substituted with a 1′-substituent. Examples of a 1′-substituent include but are not limited to fluoro, chloro, bromo, cyano, di-C₁-C₆alkylamino, C₁-C₆alkylamino, amino, C₁-C₆alkoxy and azido.

In certain embodiments, the modified sugar moiety is substituted with a 2′-substituent. In certain embodiments, the modified sugar moiety is substituted with two 2′-substituents. Examples of a 2′-position substituent include but are not limited to fluoro, chloro, bromo, cyano, di-C₁-C₆alkylamino, C₁-C₆alkylamino, amino, C₁-C₆alkoxy, azido and 2′-O(CH₂)₂OCH₃ (methoxyethyl, MOE). In certain embodiments, a sugar substituent at the 2′ position is selected from allyl, amino, azido, thio, O-allyl, O—C₁-C₁₀alkyl, OCF₃, O(CH₂)₂SCH₃, O(CH₂)₂O—N(Rm)(Rn), and O—CH₂C(═O)—N(Rm)(Rn), where each Rm and Rn is independently, H or C₁-C₁₀ alkyl.

In certain embodiments, a 2′-substituted nucleoside comprises a 2′-substituent group selected from halo (including fluoro, chloro or bromo), allyl, amino, azido, SH, CN, OCN, CF₃, OCF₃, O-alkyl, S-alkyl, or N(Ro)-alkyl; O-alkyl-O-alkyl, alkynyl, alkenyl, arylalkyl, O-arylalkyl, O(CH₂)₂SCH₃, O—(CH₂)₂O—N(Ro)(Rp) or O—CH₂C(═O)—N(Ro)(Rp), where each Ro and Rp is, independently, H, an amino protecting group or C₁-C₁₀ alkyl. A 2′-substituent group can be further substituted as allowed by valence with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂CH═CH₂, O—CH₂CH═CH₂, OCH₂CH₂OCH₃, O(CH₂)₂SCH₃, O—(CH₂)₂O—N(Rq)(Rr), O(CH₂)₂O(CH₂)₂N(CH₃)₂, and N-substituted acetamide (0-CH₂C(═O)—N(Rq)(Rr) where each Rq and Rr is independently, H, an amino protecting group or C₁-C₁₀ alkyl.

In certain embodiments, a 2′-substituted nucleoside has a sugar moiety substituted with a 2′-substituent group selected from F, OCF₃, O—CH₃, OCH₂CH₂OCH₃, O(CH₂)₂SCH₃, O—(CH₂)₂O—N(CH₃)₂, —O(CH₂)₂O(CH₂)₂N(CH₃)₂, and O—CH₂C(═O)—N(H)CH₃.

In certain embodiments, a 2′-substituted nucleoside has a sugar moiety comprising a 2′-substituent group selected from F, O—CH₃, and OCH₂CH₂OCH₃.

In certain embodiments, the modified sugar moiety is substituted with a 4′-substituent. Examples of a 4′-substituent include but are not limited to fluoro, chloro, bromo, cyano, di-C₁-C₆alkylamino, C₁-C₆alkylamino, amino, C₁-C₆alkoxy and azido.

In certain embodiments, the modified sugar moiety is substituted with a 5′-substituent. In certain embodiments, the modified sugar moiety is substituted with two 5′-substituents. Examples of a 5′-position substituent include, but are not limited to, 5′-methyl, 5′-vinyl, and 5′-methoxy. In certain embodiments, substituted sugars have more than one non-bridging sugar substituent, for example, 2′-F-5′-methyl sugar moieties (see, e.g., PCT International Application WO 2008/101157, for additional 5′, 2′-bis substituted sugar moieties and nucleosides).

In certain embodiments, the modified sugar moiety is a bicyclic sugar. In certain embodiments, the bicyclic sugar has a bridge between the 4′- and the 2′-positions. A nucleoside with a bicyclic sugar moiety is referred to as a bicyclic nucleoside or BNAs. Bicyclic nucleosides include, but are not limited to, (A) a-L-Methyleneoxy (4′-CH₂O-2′) BNA, (B) Methyleneoxy (4′-CH₂O-2′) BNA (also referred to as locked nucleic acid or LNA), (C) Ethyleneoxy (4′-(CH₂)₂O-2′) BNA, (D) Aminooxy (4′-CH₂O—N(R)-2′) BNA, (E) Oxyamino (4′-CH₂N(R)—O-2′) BNA, (F) Methyl(methyleneoxy) (4′-CH(CH₃)—O-2′) BNA (also referred to as constrained ethyl or cEt), (G) methylene-thio (4′-CH₂S-2′) BNA, (H) methylene-amino (4′-CH₂—N(R)-2′) BNA, (I) methyl carbocyclic (4′-CH₂CH(CH₃)-2′) BNA, and (J) propylene carbocyclic (4′-(CH₂)₂-2′) BNA as depicted below.

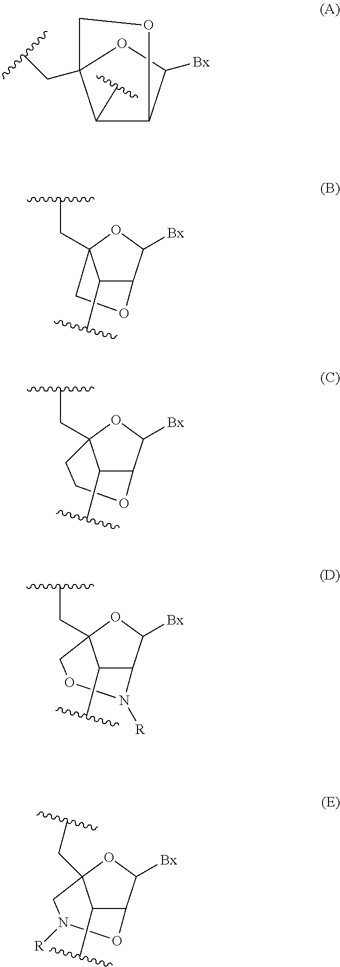

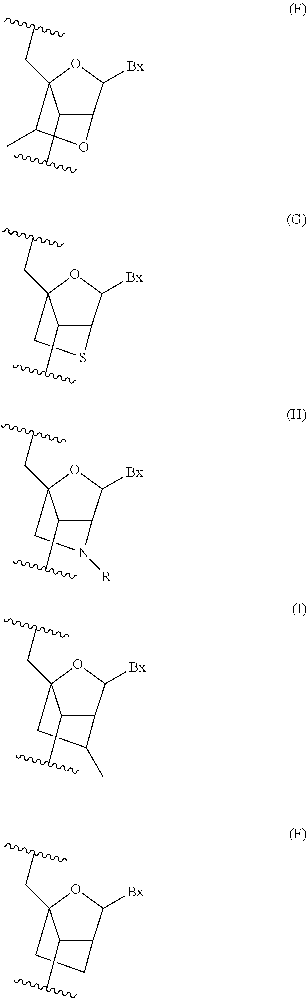

wherein Bx is Base and R is H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mal. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the L configuration or in the D configuration.

Additional nonlimiting examples of such 4′ to 2′ sugar substituents include —[C(Ra)(Rb)]ₙ—[C(Ra)(Rb)]ₙ—O—, —C(RaRb)—N(R)—O, —C(RaRb)—O—N(R)—; 4′-CH₂-2′, 4′-(CH₂)₂-2′, 4′-(CH₂)₂-2′, 4′-(CH₂)—O-2′ (LNA); 4′-(CH₂)—S-2′; 4′-(CH₂)₂O-2′ (ENA); 4′-CH(CH₃)—O-2′ (cEt) and 4′-CH(CH₂OCH₃)—O-2′, and analogs thereof (see, e.g., U.S. Pat. No. 7,399,845, issued on Jul. 15, 2008); 4′-C(CH₃)(CH₃)—O-2′ and analogs thereof, (see, e.g., WO 2009/006478, published Jan. 8, 2009); 4′-CH₂N(OCH₃)-2′ and analogs thereof (see, e.g., WO 2008/150729, published Dec. 11, 2008); 4′-CH₂O—N(CH₃)-2′ (see, e.g., US2004/0171570, published Sep. 2, 2004); 4′-CH₂O—N(R)-2′, and 4′-CH₂N(R)—O-2′-, wherein each R is independently H, a protecting group, or C₁-C₁₂ alkyl; 4′-CH₂N(R)—O-2′, wherein R is H, C₁-C₁₂ alkyl, or a protecting group (see, U.S. Pat. No. 7,427,672, issued on Sep. 23, 2008); 4′-CH₂C(H)(CH₃)-2′ (see, e.g., Chattopadhyaya, et al., J. Org. Chem., 2009, 74, 118-134); and 4′-CH₂C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Ra)(Rb)]ₙ—, —C(Ra)═C(Rb)—, —C(Ra)=N—, —C(═NRa)—, —C(═O)—, —C(═S)—, —O—, —Si(Ra)₂, —S(═O)ₓ—, and —N(Ra)—; wherein:
x is 0, 1, or 2;
n is 1, 2, 3, or 4;
each Ra and Rb is independently H, a protecting group, hydroxyl, C₁-C₁₂ alkyl, substituted C₁-C₁₂ alkyl, C₂-C₁₂ alkenyl, C₂-C₁₂ alkynyl, phenyl, heterocycle, heteroaryl, halogen, COOJ1, COJ1, acyl, S(═O)₂J1, or S(═O)J1; and
each J1 is independently H, C₁-C₁₂ alkyl, C₂-C₁₂ alkenyl, C₂-C₁₂ alkynyl, phenyl, acyl, heterocycle, C₁-C₁₂ aminoalkyl, or a protecting group.

In certain embodiments, a substituted sugar moiety is modified with a substituent and a bridge (e.g., 5′-substituted and 4′-2′ bridged sugars).

In certain embodiments, a modified sugar moiety is a sugar surrogate. In certain such embodiments, the oxygen atom of the naturally occurring sugar is substituted, e.g., with a sulfur, carbon or nitrogen atom. In certain embodiments, the sugar surrogate has bridging and/or non-bridging substituents as described above. For example, sugar surrogates can 4′-sulfur atom and a substitution at the 2′-position and/or the 5′ position (see, e.g., published U.S. Patent Application US2005/0130923). By way of additional example, carbocyclic bicyclic nucleosides having a 4′-2′ bridge have been described (see, e.g., Freier et al., Nucleic Acids Research, 1997, 25(22), 4429-4443 and Albaeketal., J. Org. Chem., 2006, 71, 7731-7740).

In certain embodiments, a sugar surrogate has a ring with other than 5-atoms. For example, in certain embodiments, a sugar surrogate comprises a morpholino. Morpholino compounds and their use in oligomeric compounds has been reported in numerous patents and published articles (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

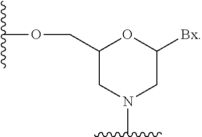

In certain embodiments, a morpholino may be modified, for example by adding or altering various substituent groups from the above morpholino structure. Such sugar surrogates are referred to herein as “modified morpholinos.”

In certain embodiments, a sugar surrogate is a tetrahydropyran. A tetrahydropyran sugar surrogate may be further modified or substituted. Nucleosides comprising modified tetrahydropyrans include, but are not limited to, hexitol nucleic acid (HNA), anitol nucleic acid (ANA), mannitol nucleic acid (MNA) (see Leumann, CJ. Bioorg. & Med. Chem. (2002) 10:841-854), fluoro HNA (F-HNA), and those compounds having Formula VI wherein Formula VI is:

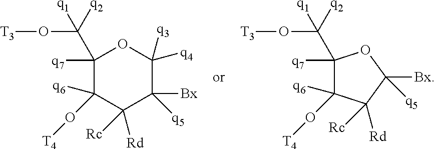

T₃ and T₄ are each, independently, an internucleoside linking group linking the tetrahydropyran nucleoside analog to the rest of the Therapeutic Oligonucleotide or one of T₃ and T₄ is an internucleoside linking group linking the tetrahydropyran nucleoside analog to the Therapeutic Oligonucleotide and the other of T₃ and T₄ is H, a hydroxyl protecting group, a linked conjugate group, or a 5′ or 3′-terminal group;
in certain embodiments T₃ and T₄ are —P(═O)(OH)—, —OP(═O)(OH)—, —P(═S)(OH)—, or —OP(═S)(OH)— wherein the O of the tetrahydropyran is attached to the P of T₃ or T₄, and bond.
q₁, q₂, q₃, q₄, q₅, q₆, and q₇, are each independently H, C₁-C₆ alkyl, C₂-C₆ alkenyl, and C₂-C₆ alkynyl; and
each of Rc and Rd is independently selected from among: hydrogen, halogen, alkoxy, NJ²H, SJ², N₃, OC(═X)J², OC(═X)NJ²H, NJ²C(═X)NJ³H and CN, wherein X is 0, S or NJ², and each J² and J³ is independently H or C₁-C₆ alkyl.

In certain embodiments, the modified THP nucleosides of Formula VI are provided wherein q1, q2, q3, q4, q5, q6 and q7 are each H. In certain embodiments, at least one of q1, q2, q3, q4, q5, q6 and q7 is other than H. In certain embodiments, at least one of q1, q2, q3, q4, q5, q6 and q7 is methyl. In certain embodiments, THP nucleosides of Formula VI are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for additional 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, the present disclosure provides a compound comprising a Therapeutic Oligonucleotide with one or more modified nucleosides. The one or more modified nucleotides may include modified sugars, modified nucleobases, and/or modified linkages. The specific modifications are selected such that the resulting oligonucleotides possess desirable characteristics, such as increased plasma stability or increased efficacy. In certain embodiments, oligonucleotides comprise one or more RNA-like nucleosides. In certain embodiments, oligonucleotides comprise one or more DNA-like nucleotides.

In certain embodiments, a modified sugar moiety replaces a modified or unmodified sugar moiety of the nucleotide sequence of the Therapeutic Oligonucleotide. In certain embodiments, a modified sugar moiety replaces a modified sugar moiety of the nucleotide sequence of the Therapeutic Oligonucleotide. In certain embodiments, a modified sugar moiety replaces an unmodified sugar moiety of the nucleotide sequence of the Therapeutic Oligonucleotide.

For example, in certain embodiments the Therapeutic Oligonucleotide corresponds to Nucleotide 1 in Table 1 wherein the first sugar of the antisense strand is replaced with an LNA sugar moiety. In other embodiments the Therapeutic Oligonucleotide corresponds to Nucleotide 1 in Table 1 wherein the first sugar of the sense strand is replaced with an LNA sugar moiety.

In certain embodiments, two modified sugar moieties replace a modified or unmodified sugar moiety of the nucleotide sequence of the Therapeutic Oligonucleotide. In certain embodiments, two modified sugar moieties replace a modified sugar moiety of the nucleotide sequence of the Therapeutic Oligonucleotide. In certain embodiments, two modified sugar moieties replace an unmodified sugar moiety of the nucleotide sequence of the Therapeutic Oligonucleotide.

In certain embodiments, three modified sugar moieties replace a modified or unmodified sugar moiety of the nucleotide sequence of the Therapeutic Oligonucleotide. In certain embodiments, three modified sugar moieties replace a modified sugar moiety of the nucleotide sequence of the Therapeutic Oligonucleotide. In certain embodiments, three modified sugar moieties replace an unmodified sugar moiety of the nucleotide sequence of the Therapeutic Oligonucleotide.

In certain embodiments, four modified sugar moieties replace a modified or unmodified sugar moiety of the nucleotide sequence of the Therapeutic Oligonucleotide. In certain embodiments, four modified sugar moieties replace a modified sugar moiety of the nucleotide sequence of the Therapeutic Oligonucleotide. In certain embodiments, four modified sugar moieties replace an unmodified sugar moiety of the nucleotide sequence of the Therapeutic Oligonucleotide.

In certain embodiments, five modified sugar moieties replace a modified or unmodified sugar moiety of the nucleotide sequence of the Therapeutic Oligonucleotide. In certain embodiments, five modified sugar moieties replace a modified sugar moiety of the nucleotide sequence of the Therapeutic Oligonucleotide. In certain embodiments, five modified sugar moieties replace an unmodified sugar moiety of the nucleotide sequence of the Therapeutic Oligonucleotide.

In certain embodiments the modified sugar is an unlocked nucleic acid (UNA). An unlocked nucleic acid has the following formula.

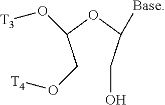

### Gapmer Sugar Motifs

In certain embodiments, the Therapeutic Oligonucleotide has a region with a gapmer sugar motif, which comprises two external regions or “wings” and a central or internal region or “gap.” The three regions of a gapmer sugar motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. For example, the nucleosides of the wings may be ribonucleosides and the nucleosides of the gap may be deoxynucleosides. Alternatively, the nucleosides of the wings may be deoxynucleosides and the nucleosides of the gap may be ribonucleosides.

Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleosides having a sugar moiety that differs from the sugar moiety of one or more other nucleosides of the gap. In certain embodiments, the sugar motifs of the two wings are the same as one another (symmetric sugar gapmer). In certain embodiments, the sugar motifs of the 5′-wing differs from the sugar motif of the 3′-wing (asymmetric sugar gapmer).

### 5′ Wings

In certain embodiments, the 5′-wing of a gapmer consists of 1 to 8 linked nucleosides. In certain embodiments, the 5′-wing of a gapmer consists of 1 to 7 linked nucleosides. In certain embodiments, the 5′-wing of a gapmer consists of 1 to 6 linked nucleosides. In certain embodiments, the 5′-wing of a gapmer consists of 1 to 5 linked nucleosides. In certain embodiments, the 5′-wing of a gapmer consists of 2 to 5 linked nucleosides. In certain embodiments, the 5′-wing of a gapmer consists of 3 to 5 linked nucleosides. In certain embodiments, the 5′-wing of a gapmer consists of 4 or 5 linked nucleosides. In certain embodiments, the 5′-wing of a gapmer consists of 1 to 4 linked nucleosides. In certain embodiments, the 5′-wing of a gapmer consists of 1 to 3 linked nucleosides. In certain embodiments, the 5′-wing of a gapmer consists of 1 or 2 linked nucleosides. In certain embodiments, the 5′-wing of a gapmer consists of 2 to 4 linked nucleosides. In certain embodiments, the 5′-wing of a gapmer consists of 2 or 3 linked nucleosides. In certain embodiments, the 5′-wing of a gapmer consists of 3 or 4 linked nucleosides. In certain embodiments, the 5′-wing of a gapmer consists of 1 nucleoside. In certain embodiments, the 5′-wing of a gapmer consists of 2 linked nucleosides. In certain embodiments, the 5′-wing of a gapmer consists of 3 linked nucleosides. In certain embodiments, the 5′-wing of a gapmer consists of 4 linked nucleosides. In certain embodiments, the 5′-wing of a gapmer consists of 5 linked nucleosides. In certain embodiments, the 5′-wing of a gapmer consists of 6 linked nucleosides.

In certain embodiments, the 5′-wing of a gapmer comprises at least one bicyclic nucleoside. In certain embodiments, the 5′-wing of a gapmer comprises at least two bicyclic nucleosides. In certain embodiments, the 5′-wing of a gapmer comprises at least three bicyclic nucleosides. In certain embodiments, the 5′-wing of a gapmer comprises at least four bicyclic nucleosides. In certain embodiments, the 5′-wing of a gapmer comprises at least one constrained ethyl nucleoside. In certain embodiments, the 5′-wing of a gapmer comprises at least one LNA nucleoside. In certain embodiments, each nucleoside of the 5′-wing of a gapmer is a bicyclic nucleoside. In certain embodiments, each nucleoside of the 5′-wing of a gapmer is a constrained ethyl nucleoside. In certain embodiments, each nucleoside of the 5′-wing of a gapmer is a LNA nucleoside.

In certain embodiments, the 5′-wing of a gapmer comprises at least one non-bicyclic modified nucleoside. In certain embodiments, the 5′-wing of a gapmer comprises at least one 2′-substituted nucleoside. In certain embodiments, the 5′-wing of a gapmer comprises at least one 2′-MOE nucleoside. In certain embodiments, the 5′-wing of a gapmer comprises at least one 2′-OMe nucleoside. In certain embodiments, each nucleoside of the 5′-wing of a gapmer is a non-bicyclic modified nucleoside. In certain embodiments, each nucleoside of the 5′-wing of a gapmer is a 2′-substituted nucleoside. In certain embodiments, each nucleoside of the 5′-wing of a gapmer is a 2′-MOE nucleoside. In certain embodiments, each nucleoside of the 5′-wing of a gapmer is a 2′-OMe nucleoside.

In certain embodiments, the 5′-wing of a gapmer comprises at least one 2′-deoxynucleoside. In certain embodiments, each nucleoside of the 5′-wing of a gapmer is a 2′-deoxynucleoside. In certain embodiments, the 5′-wing of a gapmer comprises at least one ribonucleoside. In certain embodiments, each nucleoside of the 5′-wing of a gapmer is a ribonucleoside. In certain embodiments, one, more than one, or each of the nucleosides of the 5′-wing is an RNA-like nucleoside.

In certain embodiments, the 5′-wing of a gapmer comprises at least one bicyclic nucleoside and at least one non-bicyclic modified nucleoside. In certain embodiments, the 5′-wing of a gapmer comprises at least one bicyclic nucleoside and at least one 2′-substituted nucleoside. In certain embodiments, the 5′-wing of a gapmer comprises at least one bicyclic nucleoside and at least one 2′-MOE nucleoside. In certain embodiments, the 5′-wing of a gapmer comprises at least one bicyclic nucleoside and at least one 2′-OMe nucleoside. In certain embodiments, the 5′-wing of a gapmer comprises at least one bicyclic nucleoside and at least one 2′-deoxynucleoside.

In certain embodiments, the 5′-wing of a gapmer comprises at least one constrained ethyl nucleoside and at least one non-bicyclic modified nucleoside. In certain embodiments, the 5′-wing of a gapmer comprises at least one constrained ethyl nucleoside and at least one 2′-substituted nucleoside. In certain embodiments, the 5′-wing of a gapmer comprises at least one constrained ethyl nucleoside and at least one 2′-MOE nucleoside. In certain embodiments, the 5′-wing of a gapmer comprises at least one constrained ethyl nucleoside and at least one 2′-OMe nucleoside. In certain embodiments, the 5′-wing of a gapmer comprises at least one constrained ethyl nucleoside and at least one 2′-deoxynucleoside.

### 3′ Wings

In certain embodiments, the 3′-wing of a gapmer consists of 1 to 8 linked nucleosides. In certain embodiments, the 3′-wing of a gapmer consists of 1 to 7 linked nucleosides. In certain embodiments, the 3′-wing of a gapmer consists of 1 to 6 linked nucleosides. In certain embodiments, the 3′-wing of a gapmer consists of 1 to 5 linked nucleosides. In certain embodiments, the 3′-wing of a gapmer consists of 2 to 5 linked nucleosides. In certain embodiments, the 3′-wing of a gapmer consists of 3 to 5 linked nucleosides. In certain embodiments, the 3′-wing of a gapmer consists of 4 or 5 linked nucleosides. In certain embodiments, the 3′-wing of a gapmer consists of 1 to 4 linked nucleosides. In certain embodiments, the 3′-wing of a gapmer consists of 1 to 3 linked nucleosides. In certain embodiments, the 3′-wing of a gapmer consists of 1 or 2 linked nucleosides. In certain embodiments, the 3′-wing of a gapmer consists of 2 to 4 linked nucleosides. In certain embodiments, the 3′-wing of a gapmer consists of 2 or 3 linked nucleosides. In certain embodiments, the 3′-wing of a gapmer consists of 3 or 4 linked nucleosides. In certain embodiments, the 3′-wing of a gapmer consists of 1 nucleoside. In certain embodiments, the 3′-wing of a gapmer consists of 2 linked nucleosides. In certain embodiments, the 3′-wing of a gapmer consists of 3 linked nucleosides. In certain embodiments, the 3′-wing of a gapmer consists of 4 linked nucleosides. In certain embodiments, the 3′-wing of a gapmer consists of 5 linked nucleosides. In certain embodiments, the 3′-wing of a gapmer consists of 6 linked nucleosides.

In certain embodiments, the 3′-wing of a gapmer comprises at least one bicyclic nucleoside. In certain embodiments, the 3′-wing of a gapmer comprises at least one constrained ethyl nucleoside. In certain embodiments, the 3′-wing of a gapmer comprises at least one LNA nucleoside. In certain embodiments, each nucleoside of the 3′-wing of a gapmer is a bicyclic nucleoside. In certain embodiments, each nucleoside of the 3′-wing of a gapmer is a constrained ethyl nucleoside. In certain embodiments, each nucleoside of the 3′-wing of a gapmer is a LNA nucleoside.

In certain embodiments, the 3′-wing of a gapmer comprises at least one non-bicyclic modified nucleoside. In certain embodiments, the 3′-wing of a gapmer comprises at least two non-bicyclic modified nucleosides. In certain embodiments, the 3′-wing of a gapmer comprises at least three non-bicyclic modified nucleosides. In certain embodiments, the 3′-wing of a gapmer comprises at least four non-bicyclic modified nucleosides. In certain embodiments, the 3′-wing of a gapmer comprises at least one 2′-substituted nucleoside. In certain embodiments, the 3′-wing of a gapmer comprises at least one 2′-MOE nucleoside. In certain embodiments, the 3′-wing of a gapmer comprises at least one 2′-OMe nucleoside. In certain embodiments, each nucleoside of the 3′-wing of a gapmer is a non-bicyclic modified nucleoside. In certain embodiments, each nucleoside of the 3′-wing of a gapmer is a 2′-substituted nucleoside. In certain embodiments, each nucleoside of the 3′-wing of a gapmer is a 2′-MOE nucleoside. In certain embodiments, each nucleoside of the 3′-wing of a gapmer is a 2′-OMe nucleoside.

In certain embodiments, the 3′-wing of a gapmer comprises at least one 2′-deoxynucleoside. In certain embodiments, each nucleoside of the 3′-wing of a gapmer is a 2′-deoxynucleoside. In certain embodiments, the 3′-wing of a gapmer comprises at least one ribonucleoside. In certain embodiments, each nucleoside of the 3′-wing of a gapmer is a ribonucleoside. In certain embodiments, one, more than one, or each of the nucleosides of the 5′-wing is an RNA-like nucleoside.

In certain embodiments, the 3′-wing of a gapmer comprises at least one bicyclic nucleoside and at least one non-bicyclic modified nucleoside. In certain embodiments, the 3′-wing of a gapmer comprises at least one bicyclic nucleoside and at least one 2′-substituted nucleoside. In certain embodiments, the 3′-wing of a gapmer comprises at least one bicyclic nucleoside and at least one 2′-MOE nucleoside. In certain embodiments, the 3′-wing of a gapmer comprises at least one bicyclic nucleoside and at least one 2′-OMe nucleoside. In certain embodiments, the 3′-wing of a gapmer comprises at least one bicyclic nucleoside and at least one 2′-deoxynucleoside.

In certain embodiments, the 3′-wing of a gapmer comprises at least one constrained ethyl nucleoside and at least one non-bicyclic modified nucleoside. In certain embodiments, the 3′-wing of a gapmer comprises at least one constrained ethyl nucleoside and at least one 2′-substituted nucleoside. In certain embodiments, the 3′-wing of a gapmer comprises at least one constrained ethyl nucleoside and at least one 2′-MOE nucleoside. In certain embodiments, the 3′-wing of a gapmer comprises at least one constrained ethyl nucleoside and at least one 2′-OMe nucleoside. In certain embodiments, the 3′-wing of a gapmer comprises at least one constrained ethyl nucleoside and at least one 2′-deoxynucleoside.

In certain embodiments, the 3′-wing of a gapmer comprises at least one LNA nucleoside and at least one non-bicyclic modified nucleoside. In certain embodiments, the 3′-wing of a gapmer comprises at least one LNA nucleoside and at least one 2′-substituted nucleoside. In certain embodiments, the 3′-wing of a gapmer comprises at least one LNA nucleoside and at least one 2′-MOE nucleoside. In certain embodiments, the 3′-wing of a gapmer comprises at least one LNA nucleoside and at least one 2′-OMe nucleoside. In certain embodiments, the 3′-wing of a gapmer comprises at least one LNA nucleoside and at least one 2′-deoxynucleoside.

In certain embodiments, the 3′-wing of a gapmer comprises at least one bicyclic nucleoside, at least one non-bicyclic modified nucleoside, and at least one 2′-deoxynucleoside. In certain embodiments, the 3′-wing of a gapmer comprises at least one constrained ethyl nucleoside, at least one non-bicyclic modified nucleoside, and at least one 2′-deoxynucleoside. In certain embodiments, the 3′-wing of a gapmer comprises at least one LNA nucleoside, at least one non-bicyclic modified nucleoside, and at least one 2′-deoxynucleoside.

In certain embodiments, the 3′-wing of a gapmer comprises at least one bicyclic nucleoside, at least one 2′-substituted nucleoside, and at least one 2′-deoxynucleoside. In certain embodiments, the 3′-wing of a gapmer comprises at least one constrained ethyl nucleoside, at least one 2′-substituted nucleoside, and at least one 2′-deoxynucleoside. In certain embodiments, the 3′-wing of a gapmer comprises at least one LNA nucleoside, at least one 2′-substituted nucleoside, and at least one 2′-deoxynucleoside.

In certain embodiments, the 3′-wing of a gapmer comprises at least one bicyclic nucleoside, at least one 2′-MOE nucleoside, and at least one 2′-deoxynucleoside. In certain embodiments, the 3′-wing of a gapmer comprises at least one constrained ethyl nucleoside, at least one 2′-MOE nucleoside, and at least one 2′-deoxynucleoside. In certain embodiments, the 3′-wing of a gapmer comprises at least one LNA nucleoside, at least one 2′-MOE nucleoside, and at least one 2′-deoxynucleoside.

In certain embodiments, the 3′-wing of a gapmer comprises at least one bicyclic nucleoside, at least one 2′-OMe nucleoside, and at least one 2′-deoxynucleoside. In certain embodiments, the 3′-wing of a gapmer comprises at least one constrained ethyl nucleoside, at least one 2′-OMe nucleoside, and at least one 2′-deoxynucleoside. In certain embodiments, the 3′-wing of a gapmer comprises at least one LNA nucleoside, at least one 2′-OMe nucleoside, and at least one 2′-deoxynucleoside.

### Central Regions

In certain embodiments, the gap of a gapmer consists of 6 to 20 linked nucleosides. In certain embodiments, the gap of a gapmer consists of 6 to 15 linked nucleosides. In certain embodiments, the gap of a gapmer consists of 6 to 12 linked nucleosides. In certain embodiments, the gap of a gapmer consists of 6 to 10 linked nucleosides. In certain embodiments, the gap of a gapmer consists of 6 to 9 linked nucleosides. In certain embodiments, the gap of a gapmer consists of 6 to 8 linked nucleosides. In certain embodiments, the gap of a gapmer consists of 6 or 7 linked nucleosides. In certain embodiments, the gap of a gapmer consists of 7 to 10 linked nucleosides. In certain embodiments, the gap of a gapmer consists of 7 to 9 linked nucleosides. In certain embodiments, the gap of a gapmer consists of 7 or 8 linked nucleosides. In certain embodiments, the gap of a gapmer consists of 8 to 10 linked nucleosides. In certain embodiments, the gap of a gapmer consists of 8 or 9 linked nucleosides. In certain embodiments, the gap of a gapmer consists of 6 linked nucleosides. In certain embodiments, the gap of a gapmer consists of 7 linked nucleosides. In certain embodiments, the gap of a gapmer consists of 8 linked nucleosides. In certain embodiments, the gap of a gapmer consists of 9 linked nucleosides. In certain embodiments, the gap of a gapmer consists of 10 linked nucleosides. In certain embodiments, the gap of a gapmer consists of 11 linked nucleosides. In certain embodiments, the gap of a gapmer consists of 12 linked nucleosides.

In certain embodiments, each nucleoside of the gap of a gapmer is a 2′-deoxynucleoside. In certain embodiments, the gap comprises one or more modified nucleosides. In certain embodiments, each nucleoside of the gap of a gapmer is a 2′-deoxynucleoside or is a modified nucleoside that is “DNA-like.” In certain embodiments, “DNA-like” means that the nucleoside has similar characteristics to DNA, such that a duplex comprising the gapmer and an RNA molecule is capable of activating RNase H. For example, under certain conditions, 2′-(ara)-F have been shown to support RNase H activation, and thus is DNA-like. In certain embodiments, one or more nucleosides of the gap of a gapmer is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps comprise a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

In certain embodiments, the gap comprises one or more modified nucleosides. In certain embodiments, the gap comprises one or more modified nucleosides selected from among cEt, FHNA, LNA, and 2-thio-thymidine. In certain embodiments, the gap comprises one modified nucleoside. In certain embodiments, the gap comprises a 5′-substituted sugar moiety selected from among 5′-Me, and 5′-(R)-Me. In certain embodiments, the gap comprises two modified nucleosides. In certain embodiments, the gap comprises three modified nucleosides. In certain embodiments, the gap comprises four modified nucleosides. In certain embodiments, the gap comprises two or more modified nucleosides and each modified nucleoside is the same. In certain embodiments, the gap comprises two or more modified nucleosides and each modified nucleoside is different.

In certain embodiments, the gap comprises one or more modified linkages. In certain embodiments, the gap comprises one or more methyl phosphonate linkages. In certain embodiments the gap comprises two or more modified linkages. In certain embodiments, the gap comprises one or more modified linkages and one or more modified nucleosides. In certain embodiments, the gap comprises one modified linkage and one modified nucleoside. In certain embodiments, the gap comprises two modified linkages and two or more modified nucleosides.

### Nucleobase Modifications

In certain embodiments, a Therapeutic Oligonucleotide of the present disclosure has one or more unmodified nucleobases. In certain embodiments, a Therapeutic Oligonucleotide of the present disclosure has one or more modified nucleobases.

In certain embodiments, the one or more modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—CC—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido [5,4-b][1, 4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). A modified nucleobase may also be a nucleobase in which the purine or pyrimidine base is replaced with another heterocycle, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; each of which is herein incorporated by reference in its entirety.

In certain embodiments, a Therapeutic Oligonucleotide of the present disclosure has one or more chemical modifications to one or more nucleobases arranged along the oligonucleotide or region thereof in a defined pattern or nucleobases modification motif. In certain embodiments, nucleobase modifications are arranged in a gapped motif. In certain embodiments, nucleobase modifications are arranged in an alternating motif. In certain embodiments, each nucleobase is modified. In certain embodiments, none of the nucleobases are chemically modified.

In certain embodiments, a Therapeutic Oligonucleotide of the present disclosure has a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, a nucleobase modification is a function of the natural base at a particular position of an oligonucleotide. For example, in certain embodiments each purine or each pyrimidine in an oligonucleotide is modified. In certain embodiments, each adenine is modified.

In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

In certain embodiments, chemical modifications to nucleobases include attachment of certain conjugate groups to nucleobases. In certain embodiments, each purine or each pyrimidine in an oligonucleotide may be optionally modified to comprise a conjugate group.

### Oligonucleotide Length

In certain embodiments, a Therapeutic Oligonucleotide of the present disclosure is any of a variety of ranges of lengths. In certain embodiments, oligonucleotides consist of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16 17 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

For example, in certain embodiments, the oligonucleotide may consist of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 1 7, 9 to 18, 9 to 19, 9 to 20, 9 to 21, 9 to 22, 9 to 23, 9 to 24, 9 to 25, 9 to 26, 9 to 27, 9 to 28, 9 to 29, or 9 to 30 linked nucleosides.

For example, in certain embodiments, the oligonucleotide may consist of 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, or 12 to 30 linked nucleosides.

For example, in certain embodiments, the oligonucleotide may consist of 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, or 15 to 30 linked nucleosides.

For example, in certain embodiments, the oligonucleotide may consist of 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, or 21 to 30 linked nucleosides.

For example, in certain embodiments, the oligonucleotide may consist of 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In embodiments where the number of nucleosides of an oligonucleotide of a compound is limited, whether to a range or to a specific number, the compound may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugate groups, terminal groups, or other substituents.

Further, where an oligonucleotide is described by an overall length range and by regions having specified lengths, and where the sum of specified lengths of the regions is less than the upper limit of the overall length range, the oligonucleotide may have additional nucleosides, beyond those of the specified regions, provided that the total number of nucleosides does not exceed the upper limit of the overall length range.

### Internucleoside Linkage Modifications

In a Therapeutic Oligonucleotide, an internucleoside linkage is used to link the nucleosides together. In certain embodiments, the Therapeutic Oligonucleotide comprises one or more modified linkage moieties. The two main classes of internucleoside linking groups are defined by the presence or absence of a phosphorus atom. Representative phosphorus containing internucleoside linkages include, but are not limited to, phosphodiesters (PO), phosphotriesters, methylphosphonates, phosphoramidate, and phosphorothioates (PS). Representative non-phosphorus containing internucleoside linking groups include, but are not limited to, methylenemethylimino (—CH₂N(CH₃)—O—CH₂), thiodiester (—O—C(O)—S—), thionocarbamate (—O—C(O)(NH)—S—); siloxane (—O—Si(H)₂₀—); and N,N′-dimethylhydrazine (—CH₂N(CH₃)—N(CH₃)—). Modified linkages, compared to natural phosphodiester linkages, can be used to alter and typically increase nuclease resistance of the oligonucleotide. In certain embodiments, internucleoside linkages having a chiral atom can be prepared as a racemic mixture, as separate enantiomers, or a mixture of enantiomers. In certain embodiments, the chiral phosphorus atom can be at least 80%, 85%, 90%, 95%, 98%, 99% or even 100% free of the opposite stereoisomer. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein typically contain one or more asymmetric centers and thus give rise to enantiomers, diastereomers, and other stereoisomeric configurations that may be defined, in terms of absolute stereochemistry, as (R) or (S), sugar anomers, or as (D) or (L) such as for amino acids, etc.

Neutral internucleoside linkages include without limitation, phosphotriesters, methylphosphonates, MMI (3′-CH₂N(CH₃)—O-5′), amide-3 (3′-CH₂C(═O)—N(H)-5′), amide-4 (3′—CH₂N(H)—C(═O)-5′), formacetal (3′-O—CH₂O-5′), and thioformacetal (3′-S—CH₂O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65).

In certain embodiments the Therapeutic Oligonucleotide is a Therapeutic Oligonucleotide described herein with 1, 2, 3, 4, 5, 6, 7, 8, 9, or 10 internucleoside linkage modifications.

In certain embodiments, oligonucleotides comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides of the present disclosure comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 7 phosphorothioate internucleoside linkages. In certain embodiments, the oligonucleotide comprises at least 8 phosphorothioate internucleoside linkages. In certain embodiments, the oligonucleotide comprises at least 9 phosphorothioate internucleoside linkages. In certain embodiments, the oligonucleotide comprises at least 10 phosphorothioate internucleoside linkages. In certain embodiments, the oligonucleotide comprises at least 11 phosphorothioate internucleoside linkages. In certain embodiments, the oligonucleotide comprises at least 12 phosphorothioate internucleoside linkages. In certain embodiments, the oligonucleotide comprises at least 13 phosphorothioate internucleoside linkages. In certain embodiments, the oligonucleotide comprises at least 14 phosphorothioate internucleoside linkages.

In certain embodiments, the oligonucleotide comprises at least one block of at least 6 consecutive phosphorothioate internucleoside linkages. In certain embodiments, the oligonucleotide comprises at least one block of at least 7 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 9 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide. In certain embodiments, the oligonucleotide comprises less than 15 phosphorothioate internucleoside linkages. In certain embodiments, the oligonucleotide comprises less than 14 phosphoro-thioate internucleoside linkages. In certain embodiments, the oligonucleotide comprises less than 13 phosphorothioate internucleoside linkages. In certain embodiments, the oligonucleotide comprises less than 12 phosphorothioate internucleoside linkages. In certain embodiments, the oligonucleotide comprises less than 11 phosphorothioate internucleoside linkages. In certain embodiments, the oligonucleotide comprises less than 10 phosphorothioate internucleoside linkages. In certain embodiments, the oligonucleotide comprises less than 9 phosphorothioate internucleoside linkages. In certain embodiments, the oligonucleotide comprises less than 8 phosphorothioate internucleoside linkages. In certain embodiments, the oligonucleotide comprises less than 7 phosphorothioate internucleoside linkages. In certain embodiments, the oligonucleotide comprises less than 6 phosphorothioate internucleoside linkages. In certain embodiments, the oligonucleotide comprises less than 5 phosphorothioate internucleoside linkages.

In certain embodiments the Therapeutic Oligonucleotide has chiral internucleoside linkages with high chiral purity. Non-limiting examples of techniques to prepare Therapeutic Oligonucleotides with highly pure chiral internucleoside linkages include those described in Knouse et al. “Nature Chose Phosphates and Chemists Should Too: How Emerging P(V) Methods can Augment Existing Strategies” ACS Central Science (2021) 7, 1473.

In certain embodiments one or more internucleoside linkage is

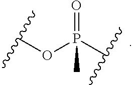 [SMILES: *O[P@](*)(C)=O]

In certain embodiments one or more internucleoside linkage is

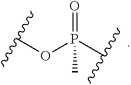 [SMILES: *O[P@@](*)(C)=O]

In certain embodiments one or more internucleoside linkage is

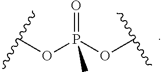

In certain embodiments one or more internucleoside linkage is

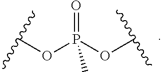

In certain embodiments one or more internucleoside linkage is

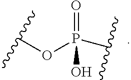 [SMILES: *O[P@@](*)(=O)O]

In certain embodiments one or more internucleoside linkage is

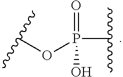 [SMILES: *O[P@](*)(=O)O]

In certain embodiments one or more internucleoside linkage is

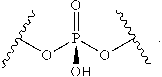

In certain embodiments one or more internucleoside linkage is

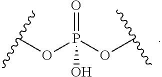

In certain embodiments one or more internucleoside linkage is

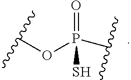 [SMILES: *O[P@@](*)(=O)S]

In certain embodiments one or more internucleoside linkage is

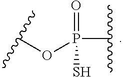 [SMILES: *O[P@](*)(=O)S]

In certain embodiments one or more internucleoside linkage is

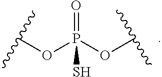

In certain embodiments one or more internucleoside linkage is

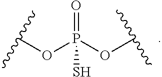

In certain embodiments one or more internucleoside linkage is

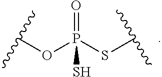 [SMILES: *O[P@@](=O)(S)S*]

In certain embodiments one or more internucleoside linkage is

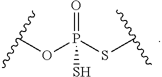 [SMILES: *O[P@](=O)(S)S*]

### Complementary Nucleotides

In certain embodiments, the Therapeutic Oligonucleotide is an antisense oligonucleotide having a sequence complementary to a target nucleic acid. Antisense compounds are capable of hybridizing to a target nucleic acid, resulting in antisense activity. In certain embodiments, antisense compounds specifically hybridize to one or more target nucleic acid sequences. In certain embodiments, a specifically hybridizing antisense compound has a nucleobase sequence comprising a region having sufficient complementarity to a target nucleic acid to allow hybridization and result in antisense activity and insufficient complementarity to an off-target so as to avoid or reduce non-specific hybridization to non-target nucleic acid sequences under conditions in which specific hybridization is desired (e.g., under physiological conditions for in vivo or therapeutic uses). In certain embodiments, oligonucleotides are selective between a target and non-target, even though both target and non-target comprise the target sequence. In such embodiments, selectivity may result from relative accessibility of the target region of one nucleic acid molecule compared to the other.

In certain embodiments, the Therapeutic Oligonucleotide is an antisense compound comprising oligonucleotides that are fully complementary to the target nucleic acid over the entire length of the oligonucleotide. In certain embodiments, the Therapeutic Oligonucleotide is 99% complementary to the target nucleic acid. In certain embodiments, the Therapeutic Oligonucleotide is 95% complementary to the target nucleic acid. In certain embodiments, the Therapeutic Oligonucleotide is 90% complementary to the target nucleic acid.

In certain embodiments, the Therapeutic Oligonucleotide is 85% complementary to the target nucleic acid. In certain embodiments, the Therapeutic Oligonucleotide is 80% complementary to the target nucleic acid. In certain embodiments, the Therapeutic Oligonucleotide comprises a region that is fully complementary to a target nucleic acid and is at least 80% complementary to the target nucleic acid over the entire length of the oligonucleotide. In certain embodiments, the region of full complementarity is from 6 to 14 nucleobases in length.

In certain embodiments, the Therapeutic Oligonucleotide comprises a hybridizing region and a terminal region. In certain such embodiments, the hybridizing region consists of 12-30 linked nucleosides and is fully complementary to the target nucleic acid. In certain embodiments, the hybridizing region includes one mismatch relative to the target nucleic acid. In certain embodiments, the hybridizing region includes two mismatches relative to the target nucleic acid. In certain embodiments, the hybridizing region includes three mismatches relative to the target nucleic acid. In certain embodiments, the terminal region consists of 1-4 terminal nucleosides. In certain embodiments, the terminal nucleosides are at the 3′ end. In certain embodiments, one or more of the terminal nucleosides are not complementary to the target nucleic acid.

Antisense mechanisms or antisense activity include(s) any mechanism involving the hybridization of an oligonucleotide with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, or splicing of the target nucleic acid.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

TABLE 1
Exemplary Therapeutic Oligonucleotides

| Nucleotide | Sequence |
|---|---|
| 1 | RNA, (Cm-s^{P-}Am-s^{P-}Gm-Am-Am-Am-(2′-deoxy-2′-fluoro) G-Am-(2′- |
| Givosiran | deoxy-2′-fluoro)G-Um-(2′-deoxy-2′-fluoro)G-Um-(2′-deoxy-2′-fluoro)C- |
| | Um-(2′-deoxy-2′-fluoro)C-Am-Um-Cm-Um-Um-Am) |
| | complex with RNA (Um-s^{P-}(2′-deoxy-2′-fluoro)A-s^{P-}(2′-deoxy-2′- |
| | fluoro) A-(2′-deoxy-2′-fluoro)G-Am-(2′-deoxy-2′-fluoro)U-Gm-(2′-deoxy- |
| | 2′-fluoro)A-Gm-(2′-deoxy-2′-fluoro)A-Cm-(2′-deoxy-2′-fluoro)A-Cm-(2′- |
| | deoxy-2′-fluoro) U-Cm-(2′-deoxy-2′-fluoro)U-Um-(2′-deoxy-2′-fluoro)U- |
| | Cm-(2′-deoxy-2′-fluoro)U-Gm-s^{P-}Gm-s^{P-}Um) |
| | cagaaagagu gucucaucuua (SEQ ID NO: 11) |
| | uaagaugaga cacucuuucuggu (SEQ ID NO: 12) |
| 2 | RNA, (Gm-s^{P-}Am-s^{P-}Cm-Um-Um-Um-(2′-deoxy-2′-fluoro)C-Am-(2′- |
| Lumasiran | deoxy-2′-fluoro)U-(2′-deoxy-2′-fluoro)C-(2′-deoxy-2′-fluoro)C-Um-Gm- |
| | Gm-Am-Am-Am-Um-Am-Um-Am), |
| | complex with RNA (Um-s^{P-}(2′-deoxy-2′-fluoro)A-s^{P-}Um-Am-Um-(2′- |
| | deoxy-2′-fluoro)U-Um-(2′-deoxy-2′-fluoro)C-(2′-deoxy-2′-fluoro)C-Am- |
| | Gm-Gm-Am-(2′-deoxy-2′-fluoro)U-Gm-(2′-deoxy-2′-fluoro)A-Am-Am- |
| | Gm-Um-Cm-s^{P-}Cm-s^{P-}Am) |
| | gacuuucauc cuggaaauaua (SEQ ID NO: 13) |
| | uauauuuccaggaugaaagucca (SEQ ID NO: 14) |
| 3 | RNA, (Am-s^{P-}(2′-deoxy-2′-fluoro)C-s^{P-}Am-(2′-deoxy-2′-fluoro)A-(2′- |
| Inclisiran | deoxy-2′-fluoro)A-(2′-deoxy-2′-fluoro)A-Gm-(2′-deoxy-2′-fluoro)C-Am- |
| | (2′-deoxy-2′-fluoro)A-Am-(2′-deoxy-2′-fluoro)A-Cm-(2′-deoxy-2′- |
| | fluoro)A-Gm-(2′-deoxy-2′-fluoro)G-Um-(2′-deoxy-2′-fluoro)C-Um-Am- |
| | Gm-s^{P-}Am-s^{P-}Am), |
| | complex with RNA (Cm-s^{P-}Um-s^{P-}Am-Gm-Am-Cm-(2′-deoxy-2′- |
| | fluoro)C-Um-(2′-deoxy-2′-fluoro)G-Um-dT-Um-Um-Gm-Cm-Um-Um- |
| | Um-Um-Gm-Um) |
| | acaaaagcaa aacaggucua gaa (SEQ ID NO: 15) |
| | cuagaccugutuugcuuuugu (SEQ ID NO: 16) |
| 4 | RNA, ((2′-deoxy-2′-fluoro)G-s^{P-}Gm-s^{P-}(2′-deoxy-2′-fluoro)U-Um-(2′- |
| Fitusiran | deoxy-2′-fluoro)A-Am-(2′-deoxy-2′-fluoro)C-Am-(2′-deoxy-2′-fluoro)C- |
| | (2′-deoxy-2′-fluoro)C-(2′-deoxy-2′-fluoro)A-Um-(2′-deoxy-2′-fluoro)U- |
| | Um-(2′-deoxy-2′-fluoro)A-Cm-(2′-deoxy-2′-fluoro)U-Um-(2′-deoxy-2′- |
| | fluoro)C-Am-(2′-deoxy-2′-fluoro)A), complex with RNA (Um-s^{P-}(2′- |
| | deoxy-2′-fluoro)U-s^{P-}Gm-(2′-deoxy-2′-fluoro)A-Am-(2′-deoxy-2′- |
| | fluoro) G-Um-(2′-deoxy-2′-fluoro)A-Am-(2′-deoxy-2′-fluoro)A-Um-Gm- |
| | Gm-(2′-deoxy-2′-fluoro) U-Gm-(2′-deoxy-2′-fluoro)U-Um-(2′-deoxy-2′- |
| | fluoro) A-Am-(2′-deoxy-2′-fluoro)C-Cm-s^{P-}Am-s^{P-}Gm) |
| | gguuaacacc auuuacuucaa (SEQ ID NO: 17) |
| | uugaaguaaaugguguuaac cag (SEQ ID NO: 18) |
| 5 | DNA, d([2′-O-(2-methoxyethyl)]m⁵rC-s^{P-}[2′-O-(2-methoxyethyl)]rG- |
| Tonlamarsen | (3′→4′)-[2′,5′-anhydro-6′-deoxy-4′-C-(hydroxymethyl)-a-L- |
| | mannofurano]m⁵C-T-s^{P-}G-s^{P-}A-s^{P-}T-s^{P-}T-s^{P-}T-s^{P-}G-s^{P-}T-s^{P}-m⁵C-s^{P-}mC- |
| | (3′→4′)-s^{P-}[2′,5′-anhydro-6′-deoxy-4′-C-(hydroxymethyl)-a-L- |
| | mannofurano]G-(3′→4′)-[2′,5′-anhydro-6′-deoxy-4′-C-(hydroxymethyl)-α- |
| | L-mannofurano]G-s^{P-}[2′-O-(2-methoxyethyl)]rG) |
| | cgctgatttg tccggg (SEQ ID NO: 19) |
| 6 | RNA, (Am-s^{P-}Am-s^{P-}(2′-deoxy-2′-fluoro)G-Cm-(2′-deoxy-2′-fluoro)A- |
| Cemdisiran | Am-(2′-deoxy-2′-fluoro)G-Am-(2′-deoxy-2′-fluoro)U-(2′-deoxy-2′- |
| | fluoro)A-(2′-deoxy-2′-fluoro)U-Um-(2′-deoxy-2′-fluoro) U-Um-Um-(2′- |
| | deoxy-2′-fluoro)A-Um-(2′-deoxy-2′-fluoro)A-Am-Um-Am), complex with |
| | RNA (Um-s^{P-}(2′-deoxy-2′-fluoro)A-s^{P-}(2′-deoxy-2′-fluoro)U-Um-(2′- |
| | deoxy-2′-fluoro)A-Um-Am-(2′-deoxy-2′-fluoro)A-Am-(2′-deoxy-2′- |
| | fluoro) A-Am-Um-Am-(2′-deoxy-2′-fluoro)U-Cm-(2′-deoxy-2′-fluoro)U- |
| | Um-(2′-deoxy-2′-fluoro)G-Cm-Um-Um-s^{P-}Um-s^{P-}Um-dT-dT) |
| | aagcaagaua uuuuuauaau a (SEQ ID NO: 20) |
| | uauuauaaaa auaucuugcuuuutt (SEQ ID NO: 21) |
| 7 | DNA, d([2′-O-(2-methoxyethyl)]mrU-s^{P-}[2′-O-(2-methoxyethyl)]rG-s^{P-} |
| Donidalorsen | [2′-O-(2-methoxyethyl)]m⁵rC-[2′-O-(2-methoxyethyl)]rA-[2′-O-(2- |
| | methoxyethyl) ]rA-s^{P-}G-s^{P-}T-s^{P-}m⁵C-s^{P-}T-s^{P-}m⁵C-s^{P-}T-s^{P-}T-s^{P-}G-s^{P-}G-s^{P-} |
| | m⁵C-s^{P-}[2′-O-(2-methoxyethyl)]rA-[2′-O-(2-methoxyethyl)]rA-[2′-O-(2- |
| | methoxyethyl)]rA-s^{P-}[2′-O-(2-methoxyethyl)]m⁵rC-s^{P-}[2′-O-(2- |
| | methoxyethyl)]rA) |
| | ugcaagtctc ttggcaaaca (SEQ ID NO: 22) |
| 8 | RNA, (Gm-s^{P-}Um-s^{P-}Gm-Um-(2′-deoxy-2′-fluoro)G-Cm-(2′-deoxy-2′- |
| ALN-HBV02 | fluoro)A-(2′-deoxy-2′-fluoro)C-(2′-deoxy-2′-fluoro)U-Um-Cm-Gm-Cm- |
| | Um-Um-Cm-Am-Cm-Am), |
| | complex with RNA (Um-s^{P-}(2′-deoxy-2′-fluoro)G-s^{P-}Um-Gm-Am- |
| | (3′→3′)-[9-de-B-D-ribofuranosyl-9-[(2S)-2,3-dihydroxypropyl]]A- |
| | (2′→5′)-Gm-(2′-deoxy-2′-fluoro)C-(2′-deoxy-2′-fluoro)G-Am-Am-Gm- |
| | Um-(2′-deoxy-2′-fluoro)G-Cm-(2′-deoxy-2′-fluoro)A-Cm-Am-Cm-s^{P-}Um- |
| | s^{P-}Um) |
| | gugugcacuu cgcuucaca (SEQ ID NO: 23) |
| | ugugaagcgaagugcacacu u (SEQ ID NO: 24) |
| 9 | RNA, (Gm-s^{P-}Um-s^{P-}Cm-Am-Um-Cm-(2′-deoxy-2′-fluoro)C-Am-(2′- |
| ALN-AGT | deoxy-2′-fluoro)C-(2′-deoxy-2′-fluoro)A-(2′-deoxy-2′-fluoro)A-Um-Gm- |
| | Am-Gm-Am-Gm-Um-Am-Cm-Am), |
| | complex with RNA (Um-s^{P-}(2′-deoxy-2′-fluoro)G-s^{P-}Um-Am-Cm- |
| | (3′→3′)-[1-de-B-D-ribofuranosy1-1-[(2S)-2,3-dihydroxypropyl]]m⁵U- |
| | (2′→5′)-Cm-Um-Cm-Am-Um-Um-Gm-(2′-deoxy-2′-fluoro)U-Gm-(2′- |
| | deoxy-2′-fluoro) G-Am-Um-Gm-Am-Cm-s^{P-}Gm-s^{P-}Am) |
| | gucauccaca augagaguaca (SEQ ID NO: 25) |
| | uguacucucauuguggauga cga (SEQ ID NO: 26) |
| 10 | RNA, (Am-s^{P-}(2′-deoxy-2′-fluoro)G-s^{P-}Am-Um-Am-Gm-(3′-+3′)-[1-de-ß- |
| ALN-HSD | D-ribofuranosyl-1-[(2S)-2,3-dihydroxypropyl]]m⁵U-(2′→5′)-Cm-Cm-Am- |
| | Um-Gm-Cm-(2′-deoxy-2′-fluoro)A-Am-(2′-deoxy-2′-fluoro)A-Am-Gm- |
| | Cm-Am-Um-s^{P-}Um-s^{P-}Cm), |
| | complex with RNA (Am-s^{P-}Um-s^{P-}Gm-Cm-Um-Um-(2′-deoxy-2′- |
| | fluoro)U-Um-(2′-deoxy-2′-fluoro)G-(2′-deoxy-2′-fluoro)C-(2′-deoxy-2′- |
| | fluoro)A-Um-Gm-Gm-Am-Cm-Um-Am-Um-Cm-Um) |
| | agauagucca ugcaaaagca uuc (SEQ ID NO: 27) |
| | augcuuuugc auggacuaucu (SEQ ID NO: 28) |
| 11 | RNA, (Cm-s^{P-}Am-Gm-Cm-Cm-Cm-Cm-Um-(2′-deoxy-2′-fluoro)U-(2′- |
| Olpasiran | deoxy-2′-fluoro)A-(2′-deoxy-2′-fluoro)U-Um-Gm-Um-Um-Am-Um-Am- |
| | Cm-Gm-(3′-+3′)-s^{P-}dA), |
| | complex with RNA (Um-s^{P-}(2′-deoxy-2′-fluoro)C-s^{P-}Gm-(2′-deoxy-2′- |
| | fluoro) U-Am-(2′-deoxy-2′-fluoro) U-Am-Am-Cm-Am-Am-(2′-deoxy-2′- |
| | fluoro)U-Am-(2′-deoxy-2′-fluoro)A-Gm-(2′-deoxy-2′-fluoro)G-Gm-(2′- |
| | deoxy-2′-fluoro)G-Cm-s^{P-}(2′-deoxy-2′-fluoro)U-s^{P-}Gm) |
| | cagccccuua uuguuauacg a (SEQ ID NO: 29) |
| | ucguauaaca auaaggggcug (SEQ ID NO: 30) |
| 12 | DNA, d([2′-O-(2-methoxyethyl)]rA-s^{P-}[2′-O-(2-methoxyethyl)]m⁵rC-[2′- |
| Fesomersen | O-(2-methoxyethyl)]rG-[2′-O-(2-methoxyethyl)]rG-[2′-O-(2- |
| | methoxyethyl)]m⁵rC-A-s^{P-}T-s^{P-}T-s^{P-}G-s^{P-}G-s^{P-}T-s^{P-}G-s^{P-}m⁵C-s^{P-}A-sp_ |
| | m⁵C-s^{P-}[2′-O-(2-methoxyethyl)]rA-[2′-O-(2-methoxyethyl)]rG-[2′-O-(2- |
| | methoxyethyl)]m⁵rU-s^{P-}[2′-O)-(2-methoxyethyl)]m⁵rU-s^{P-}[2′-O)-(2- |
| | methoxyethyl)]m⁵rU) |
| | acggcattgg tgcacaguuu (SEQ ID NO: 31) |
| 13 | RNA, (Am-s^{P-}Um-(2′-deoxy-2′-fluoro)G-Um-(2′-deoxy-2′-fluoro)U-Gm- |
| Nedosiran | Um-(2′-deoxy-2′-fluoro)C-(2′-deoxy-2′-fluoro)C-(2′-deoxy-2′-fluoro)U- |
| | (2′-deoxy-2′-fluoro)U-Um-(2′-deoxy-2′-fluoro)U-Um-(2′-deoxy-2′- |
| | fluoro)A-Um-(2′-deoxy-2′-fluoro)C-Um-Gm-Am-Gm-Cm-Am-Gm-Cm- |
| | Cm-G-A-A-A-Gm-Gm-Cm-Um-Gm-Cm), |
| | complex with RNA ([1-de-B-D-ribofuranosyl-1-[(2R,3R,4S,5R)-tetrahydro- |
| | 4-hydroxy-5-[(hydroxymethoxyphosphinyl)methoxy]-3-methoxy-2- |
| | furany1]]U-(4′→5′)-s^{P-}(2′-deoxy-2′-fluoro)C-s^{P-}(2′-deoxy-2′-fluoro)A-s^{P-} |
| | (2′-deoxy-2′-fluoro)G-(2′-deoxy-2′-fluoro)A-Um-(2′-deoxy-2′-fluoro)A- |
| | Am-(2′-deoxy-2′-fluoro)A-(2′-deoxy-2′-fluoro)A-Am-Gm-Gm-(2′-deoxy- |
| | 2′-fluoro)A-Cm-(2′-deoxy-2′-fluoro)A-Am-(2′-deoxy-2′-fluoro)C-Am- |
| | Um-s^{P-}Gm-s^{P-}Gm) |
| | auguuguccu uuuuaucuga gcagccgaaa ggcugc (SEQ ID NO: 32) |
| | ucagauaaaa aggacaacau gg (SEQ ID NO: 33) |
| 14 | RNA, (Gm-s^{P-}Am-(2′-deoxy-2′-fluoro)C-Am-Am-Am-Am-(2′-deoxy-2′- |
| Xalnesiran | fluoro)A-(2′-deoxy-2′-fluoro)U-(2′-deoxy-2′-fluoro)C-Cm-(2′-deoxy-2′- |
| | fluoro) U-(2′-deoxy-2′-fluoro)C-Am-Cm-Am-(2′-deoxy-2′-fluoro)A-Um- |
| | Am-Am-Gm-Cm-Am-Gm-Cm-Cm-G-A-A-A-Gm-Gm-Cm-Um-Gm-Cm), |
| | complex with RNA ([4′-de(hydroxymethyl)-4′- |
| | [(hydroxymethoxyphosphinyl)methoxy ]]Um-s^{P-}(2′-deoxy-2′-fluoro)U-s^{P-} |
| | (2′-deoxy-2′-fluoro)A-s^{P-}Um-(2′-deoxy-2′-fluoro)U-Gm-(2′-deoxy-2′- |
| | fluoro) U-(2′-deoxy-2′-fluoro)G-Am-(2′-deoxy-2′-fluoro)G-Gm-(2′-deoxy- |
| | 2′-fluoro)A-Um-(2′-deoxy-2′-fluoro)U-Um-(2′-deoxy-2′-fluoro)U-Um- |
| | Gm-(2′-deoxy-2′-fluoro)U-Cm-s^{P-}Gm-s^{P-}Gm) |
| | gacaaaaauc cucacaauaa gcagccgaaa ggcugc (SEQ ID NO: 34) |
| | uuauugugag gauuuuuguc gg (SEQ ID NO: 35) |
| 15 | RNA, (Am-s^{P-}(2′-deoxy-2′-fluoro)U-s^{P-}Am-(2′-deoxy-2′-fluoro)A-Cm-(2′- |
| Zerlasiran | deoxy-2′-fluoro) U-Cm-(2′-deoxy-2′-fluoro)U-Gm-(2′-deoxy-2′-fluoro)U- |
| | Cm-(2′-deoxy-2′-fluoro)C-Am-(2′-deoxy-2′-fluoro)U-Um-(2′-deoxy-2′- |
| | fluoro) A-Cm-s^{P-}(2′-deoxy-2′-fluoro)C-s^{P-}Gm), |
| | complex with RNA (Cm-Gm-Gm-Um-Am-Am-(2′-deoxy-2′-fluoro)U-(2′- |
| | deoxy-2′-fluoro)G-(2′-deoxy-2′-fluoro)G-Am-Cm-Am-Gm-Am-Gm-Um- |
| | Um-s^{P-}Am-s^{P-}Um) |
| | auaacucugu ccauuaccg (SEQ ID NO: 36) |
| | cgguaaugga cagaguuau (SEQ ID NO: 37) |
| 16 | DNA, d([2′-O-(2-methoxyethyl)]m⁵rU-s^{P-}[2′-O)-(2-methoxyethyl)]rG-[2′- |
| Pelacarsen | O-(2-methoxyethyl)]m⁵rC-[2′-O-(2-methoxyethyl)]m⁵rU-[2′-O-(2- |
| | methoxyethyl)]morC-m⁵C-s^{P-}G-s^{P-}T-s^{P-}T-s^{P-}G-s^{P-}G-s^{P-}T-s^{P-}G-s^{P-}m⁵C-sp_ |
| | T-s^{P-}[2′-O-(2-methoxyethyl)]m⁵rU-[2′-O-(2-methoxyethyl)]rG-[2′-O-(2- |
| | methoxyethyl)]m⁵rU-s^{P-}[2′-O-(2-methoxyethyl)]m⁵rU-s^{P-}[2′-O-(2- |
| | methoxyethyl)]m⁵rC) |
| | tgctccgttg gtgcttgttc (SEQ ID NO: 38) |
| 17 | DNA, d(P-thio)([2′-O-(2-methoxyethyl)]rA-[2′-O-(2-methoxyethyl)]rG- |
| IONIS- | [2′-O-(2-methoxyethyl)]m⁵rC-[2′-O-(2-methoxyethyl)]m⁵rU-[2′-O-(2- |
| APOCIII- | methoxyethyl)]m⁵rU-m⁵C-T-T-G-T-m⁵C-m⁵C-A-G-m⁵C-[2′-O-(2- |
| LRx | methoxyethyl)]m⁵rU-[2′-O-(2-methoxyethyl)]m⁵rU-[2′-O-(2- |
| | methoxyethyl)]m⁵rU-[2′-O-(2-methoxyethyl)]rA-[2′-O-(2- |
| | methoxyethyl)]m⁵rU) |
| | agcuucttgtccagcuuuau (SEQ ID NO: 39) |
| 18 | DNA, d(P-thio)([2′-O-(2-methoxyethyl)]rA-[2′-O-(2- |
| IONIS- | methoxyethyl)]m⁵rU-[2′-O-(2-methoxyethyl)]m⁵rC-[2′-O-(2- |
| FB-LRx | methoxyethyl)]m⁵rC-[2′-O-(2-methoxyethyl)]m⁵rC-A-m⁵C-G-m⁵C-m⁵C- |
| | m⁵C-m⁵C-T-G-T-[2′-O-(2-methoxyethyl)]m⁵rC-[2′-O-(2- |
| | methoxyethyl)]morC-[2′-O-(2-methoxyethyl)]rA-[2′-O-(2- |
| | methoxyethyl)]rG-[2′-O-(2-methoxyethyl)]m⁵rC) |
| | aucccacgcc cctgtccagc (SEQ ID NO: 40) |
| 19 | Sense Strand 5′-3′ |
| | (invAb)sguggacuuCfUfCfucaauuuucus(invAb) |
| | Antisense Strand 5′-3′ |
| | asGfsasAfaAfuUfgAfgAfgAfaGfuCfcasc |
| | GUGGACUUCUCUCAAUUUUCU (SEQ ID NO: 41) |
| | AGAAAAUUGAGAGAAGUCCAC (SEQ ID NO: 42) |
| 20 | Sense Strand 5′-3′ |
| | (invAb)scgcuguagGfCfAfuaaauugguas(invAb) |
| | Antisense Strand 5′-3′ |
| | usAfscsCfaAfuUfuAfuGfcCfuAfcAfgcsg |
| | CGCUGUAGGCAUAAAUUGGUA (SEQ ID NO: 43) |
| | UACCAAUUUAUGCCUACAGCG (SEQ ID NO: 44) |
| 21 | ^{m}CₑₛGₑₒ^{m}CₖₒT_{ds}G_{ds}A_{ds}T_{ds}T_{ds}T_{ds}G_{ds}T_{ds}^{m}C_{ds}^{m}C_{ds}GₖₒGₖₛGₑ |
| | (SEQ ID NO: 45) |
| | wherein |
| | A is an adenine nucleobase; |
| | ^{m}C is a 5-methyl cytosine nucleobase; |
| | G is a guanine nucleobase; |
| | T is a thymine nucleobase; |
| | e is a 2′-b-D-MOE sugar moiety; |
| | k is a cEt sugar moiety; |
| | dis a 2′-b-D-deoxyribosyl sugar moiety; |
| | s is a phosphorothioate internucleoside linkage; and |
| | o is a phosphodiester internucleoside linkage. |
| 22 | Sense Strand 5′-3′ |
| | gsusgcACUucgcuucaca (SEQ ID NO: 46) |
| | Antisense Strand 5′-3′ |
| | usGsugU(a)agcgaaguGcAcacsgsgr(u) (SEQ ID NO: 47) |
| | wherein 2′-O-Methyl nucleotides = lower case; 2′-Fluoro nucleotides = |
| | UPPER CASE; Phosphorothioate linker = s: Unmodified = r(nucleotide), |
| | UNA = U(nucleotide) |
| 23 | Sense Strand 5′-3′ |
| | gsusgcACUucgcuucaca (SEQ ID NO: 48) |
| | Antisense Strand 5′-3′ |
| | usGsugaU(a)gcgaaguGcAcacsgsgr(u) (SEQ ID NO: 49) |
| | wherein 2′-O-Methyl nucleotides = lower case; 2′-Fluoro nucleotides = |
| | UPPER CASE; Phosphorothioate linker = s: Unmodified = r(nucleotide), |
| | UNA = U(nucleotide) |
| 24 | Sense Strand 5′-3′ |
| | gsusgcACUucgcuucaca (SEQ ID NO: 50) |
| | Antisense Strand 5′-3′ |
| | usGsugaagcgaaguGcAcacsgsgr(u) (SEQ ID NO: 51) |
| | wherein 2′-O-Methyl nucleotides = lower case; 2′-Fluoro nucleotides = |
| | UPPER CASE; Phosphorothioate linker = s: Unmodified = r(nucleotide), |
| | UNA = U(nucleotide) |

In certain embodiments the Therapeutic Oligonucleotide is givosiran.

In certain embodiments the Therapeutic Oligonucleotide is lumasiran.

In certain embodiments the Therapeutic Oligonucleotide is inclisiran.

In certain embodiments the Therapeutic Oligonucleotide is fitusiran.

In certain embodiments the Therapeutic Oligonucleotide is cemdisiran.

In certain embodiments the Therapeutic Oligonucleotide is ALN-HBV02.

In certain embodiments the Therapeutic Oligonucleotide is ALN-AGT.

In certain embodiments the Therapeutic Oligonucleotide is ALN-HSD.

In certain embodiments the Therapeutic Oligonucleotide is olpasiran.

In certain embodiments the Therapeutic Oligonucleotide is nedosiran.

In certain embodiments the Therapeutic Oligonucleotide is xalnesiran.

In certain embodiments the Therapeutic Oligonucleotide is zerlasiran.

In certain embodiments the Therapeutic Oligonucleotide is pelacarsen.

In certain embodiments the Therapeutic Oligonucleotide is IONIS-APOCIII-L_{Rx}.

In certain embodiments the Therapeutic Oligonucleotide is IONIS-FB-L_{Rx}.

In certain embodiments the Therapeutic Oligonucleotide is fesomersen.

In certain embodiments the Therapeutic Oligonucleotide is donidalorsen.

In certain embodiments the Therapeutic Oligonucleotide is tonlamarsen.

In certain embodiments the Therapeutic Oligonucleotide is ARO-HBV.

In certain embodiments the Therapeutic Oligonucleotide is IONIS-AGT-L_{Rx}.

In certain embodiments the Therapeutic Oligonucleotide is AB-729.

In certain embodiments the Therapeutic Oligonucleotide is SLN-124.

When an embodiment, formula, or claim refers to a Therapeutic Oligonucleotide by name and that therapeutic nucleotide has a GalNAc delivery component, then the embodiment, formula, or claim refers to the oligonucleotide portion of the molecule. Similarly, when an embodiment, formula, or claim refers to a therapeutic nucleotide with a phosphate group at the attaching point to the cleavable moiety and that structure, embodiment, or claim already has a drawn phosphate group or portion of a phosphate group the skilled artisan will understand that the drawn or claimed structure has one phosphate group in that position. For example, Compound 1 is drawn in the following shorthand forms in this application for convenience

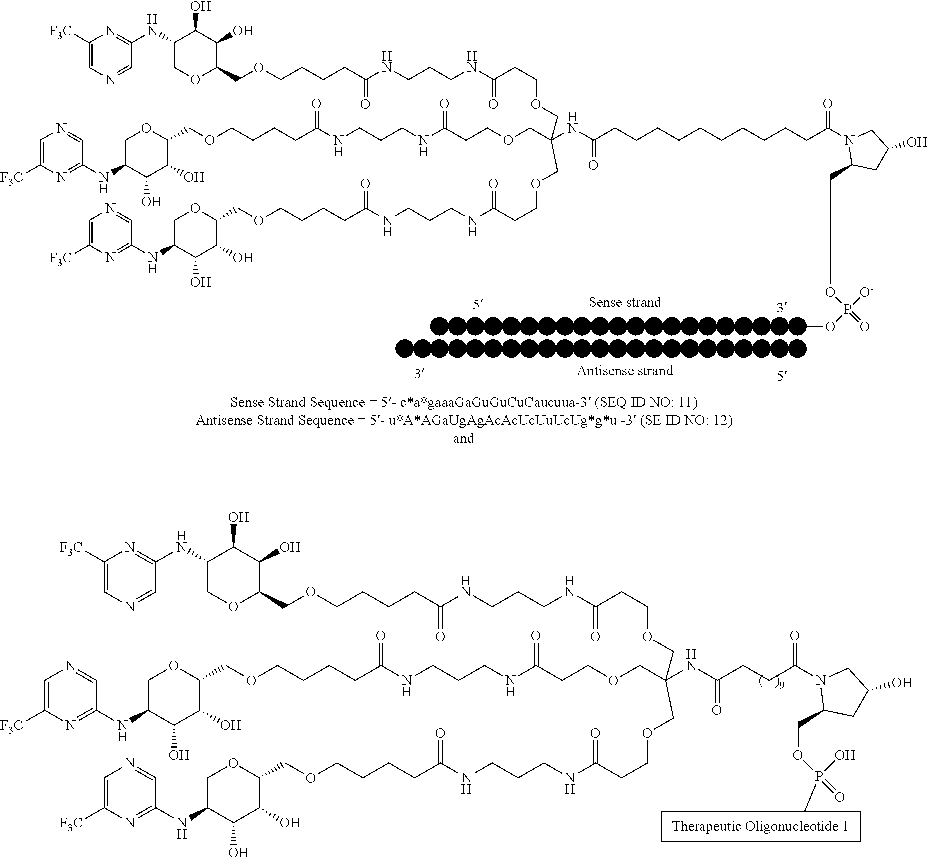

the skilled artisan will recognize, and it is contemplated in this application, that the drawn phosphate group attached to the 3′ sense strand is the phosphate of that sequence and that the resulting structure is:

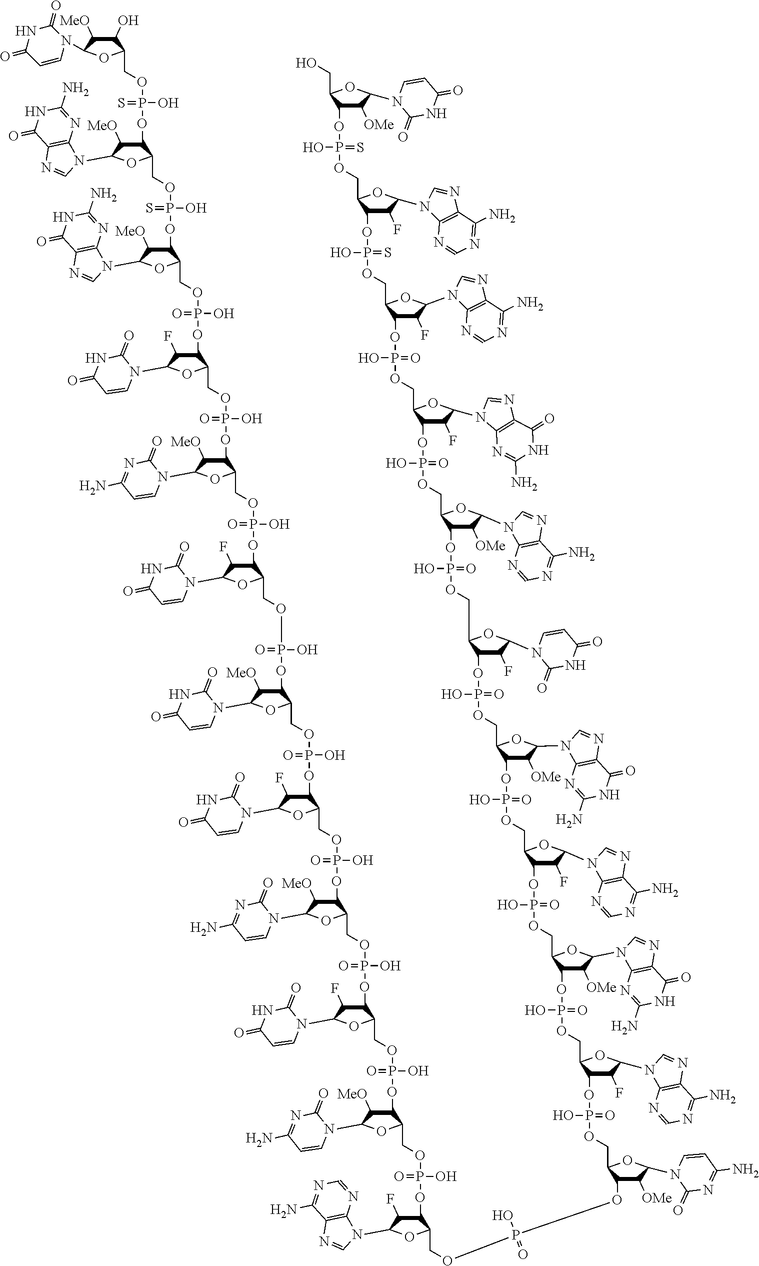

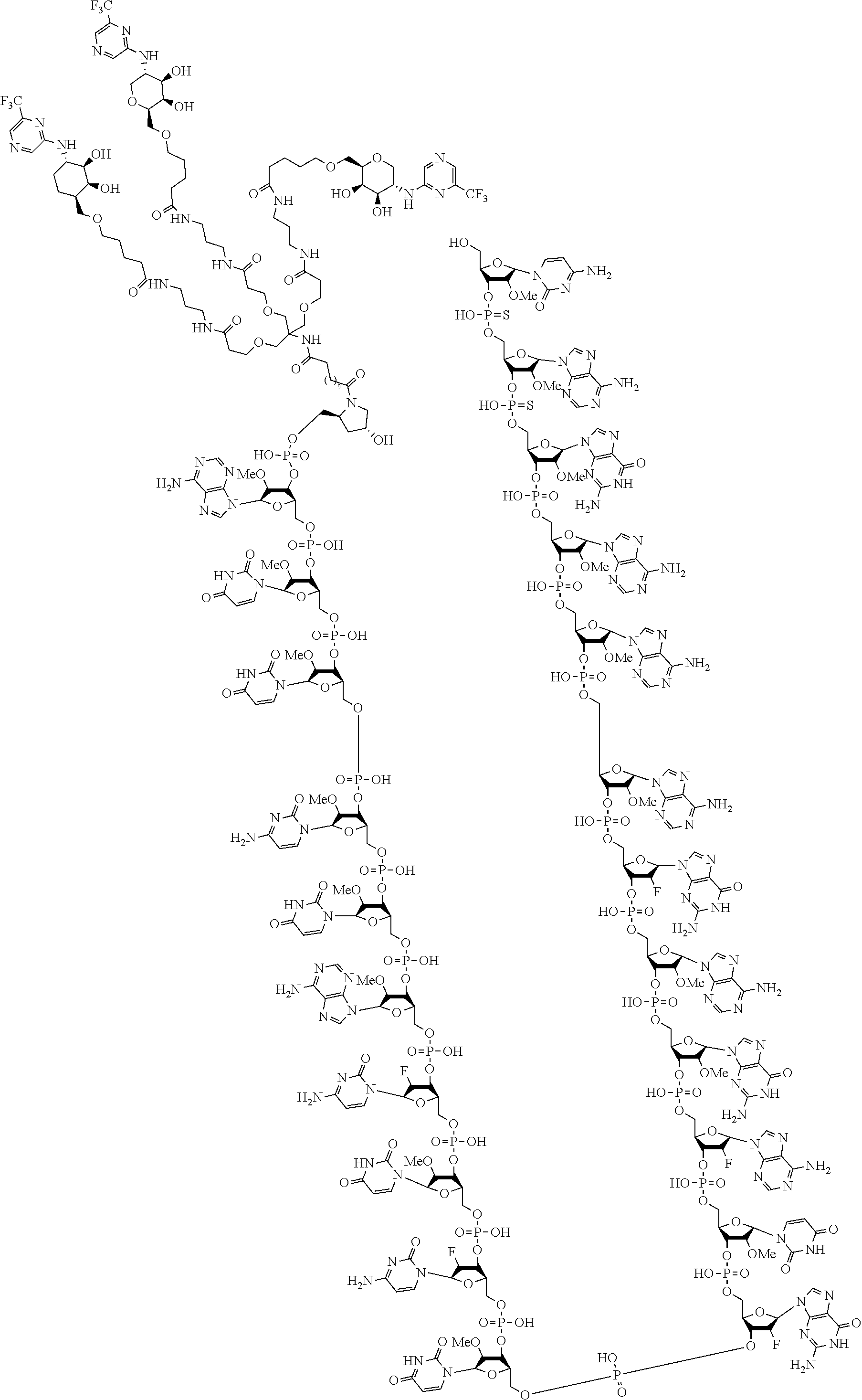

In certain embodiments, the Therapeutic Oligonucleotide delivering compound of the present invention is

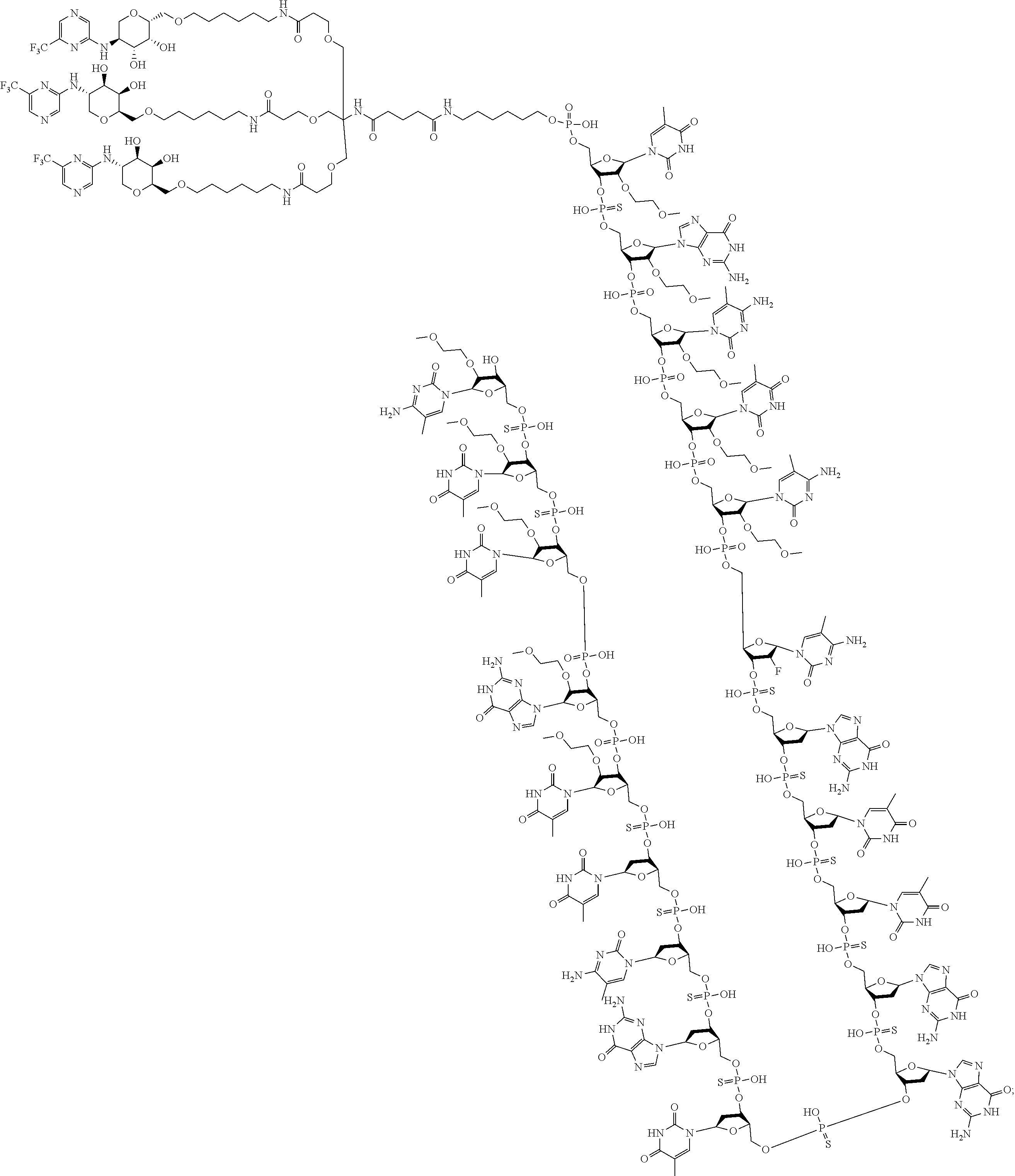

or a pharmaceutically acceptable salt thereof.

In certain embodiments, the Therapeutic Oligonucleotide delivering compound of the present invention is

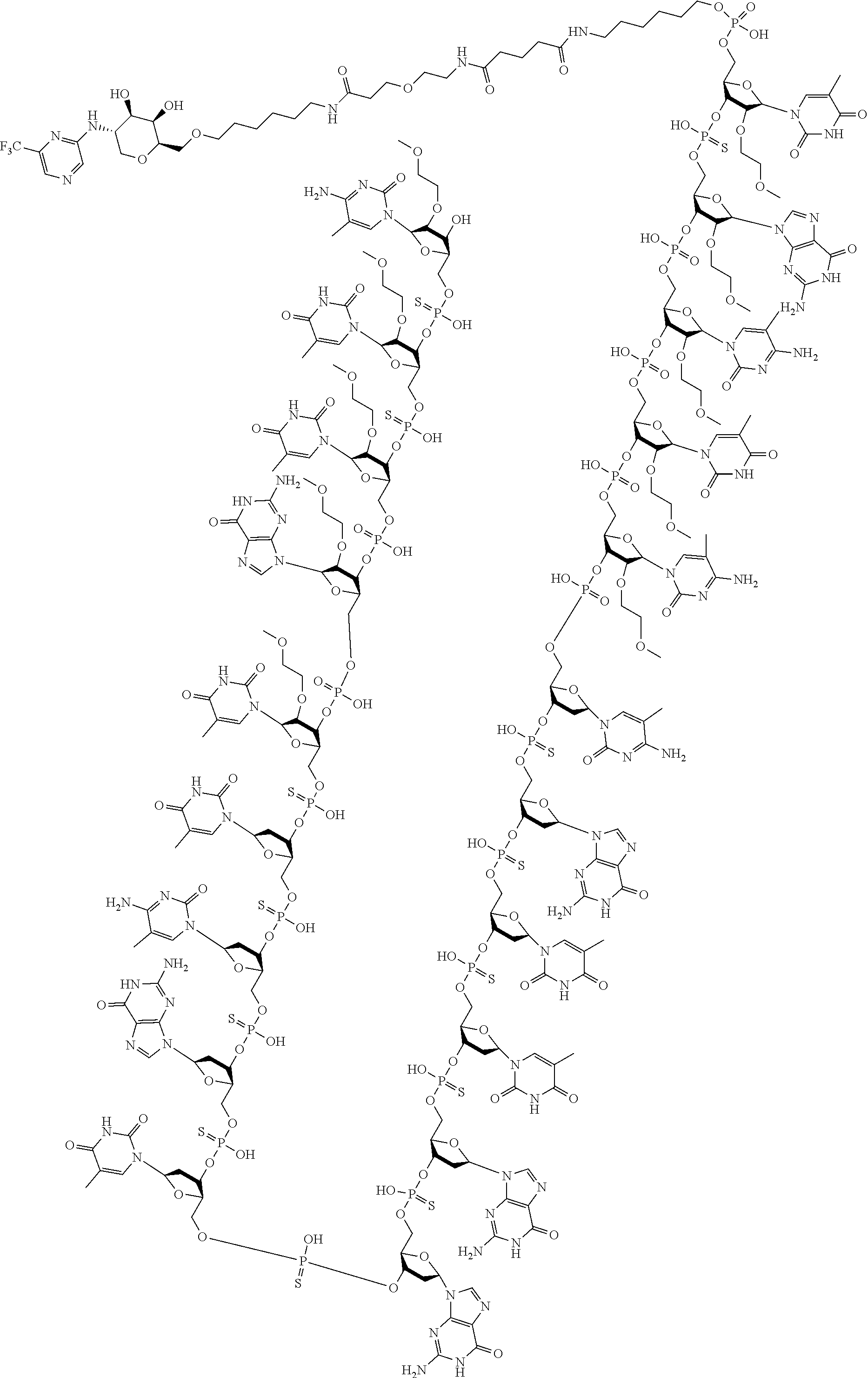

or a pharmaceutically acceptable salt thereof.

In certain embodiments, the Therapeutic Oligonucleotide delivering compound of the present invention is:

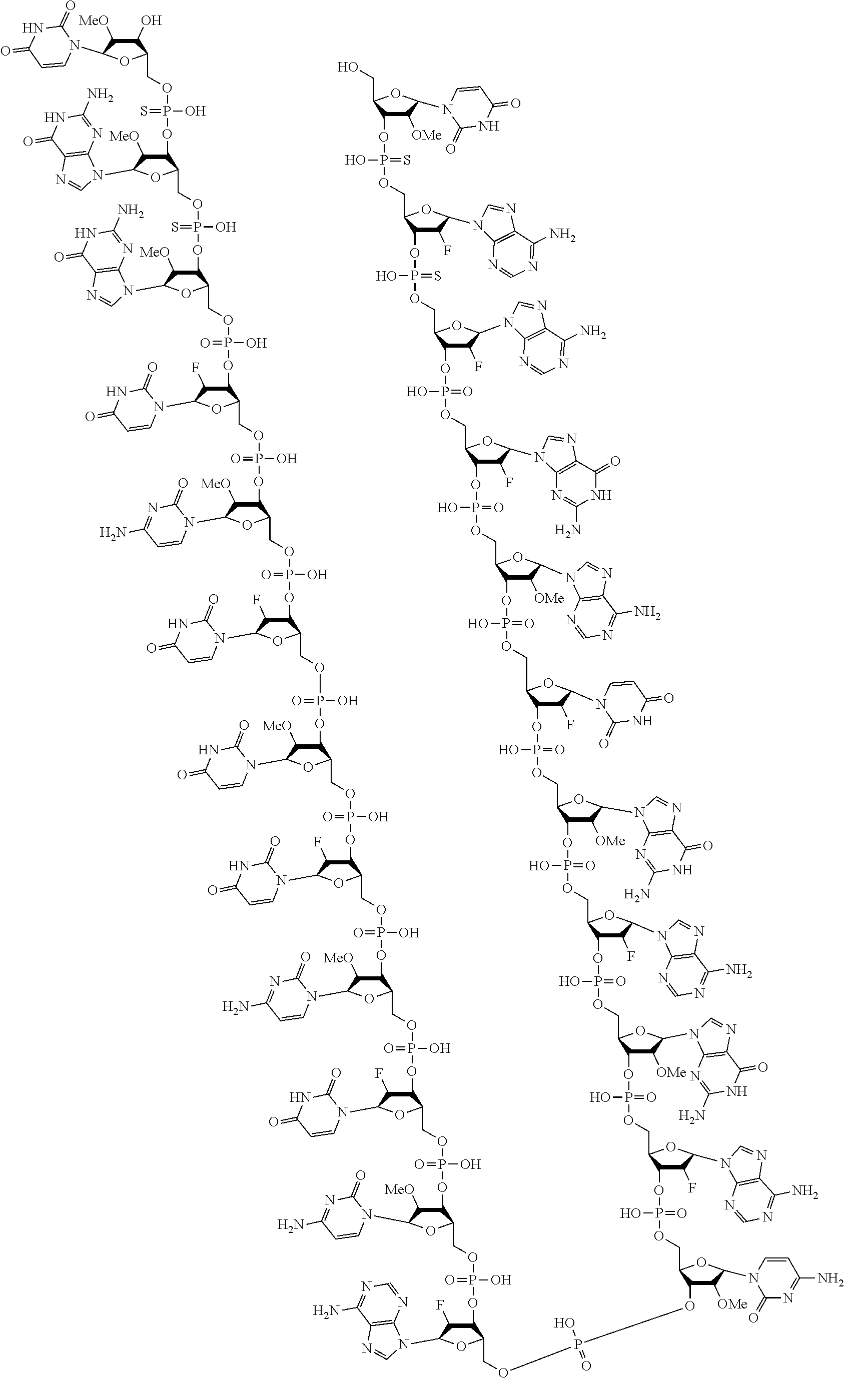

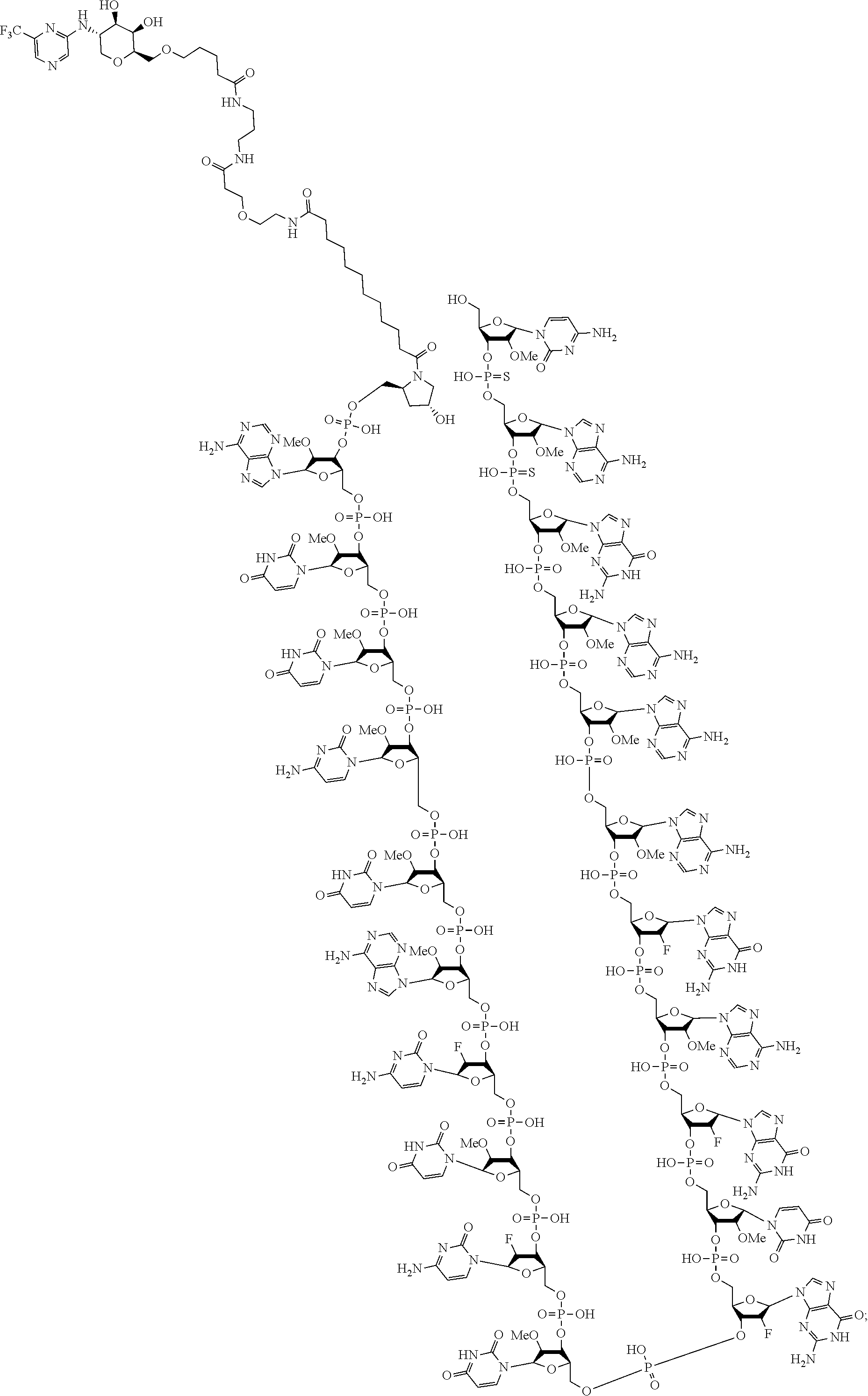

or a pharmaceutically acceptable salt thereof.

In certain embodiments, the Therapeutic Oligonucleotide delivering compound of the present invention is:

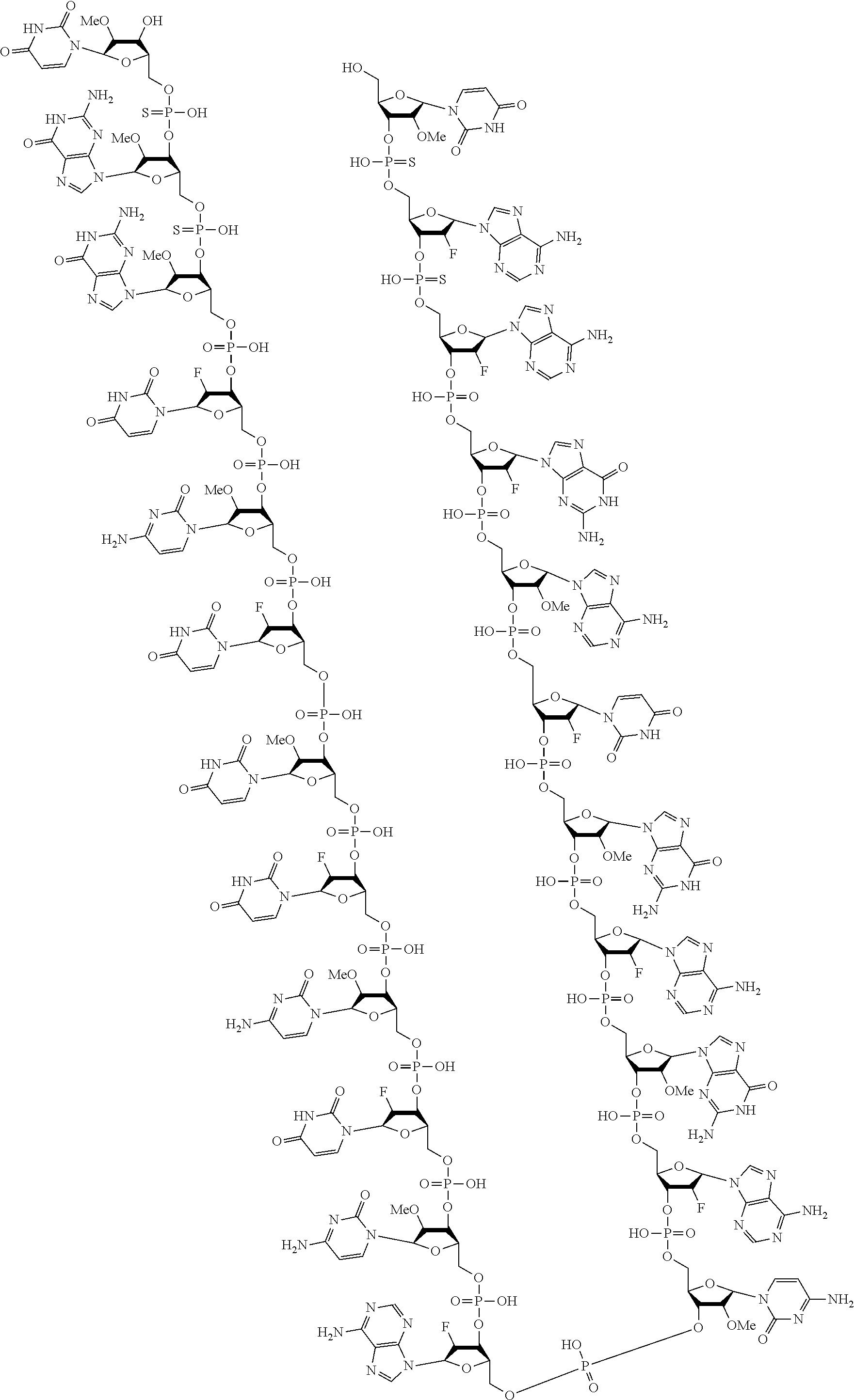

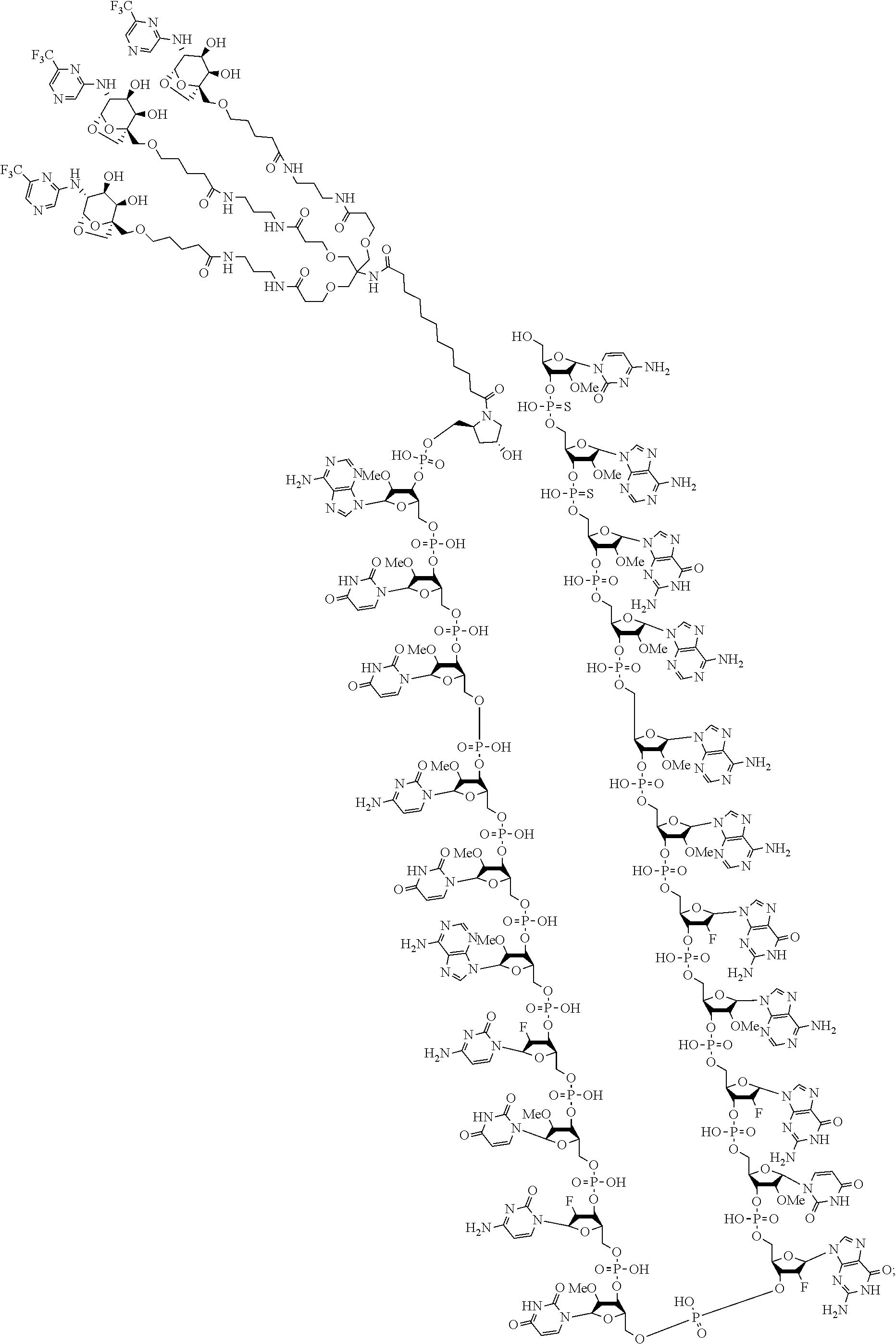

or a pharmaceutically acceptable salt thereof.

In certain embodiments, the Therapeutic Oligonucleotide delivering compound of the present invention is:

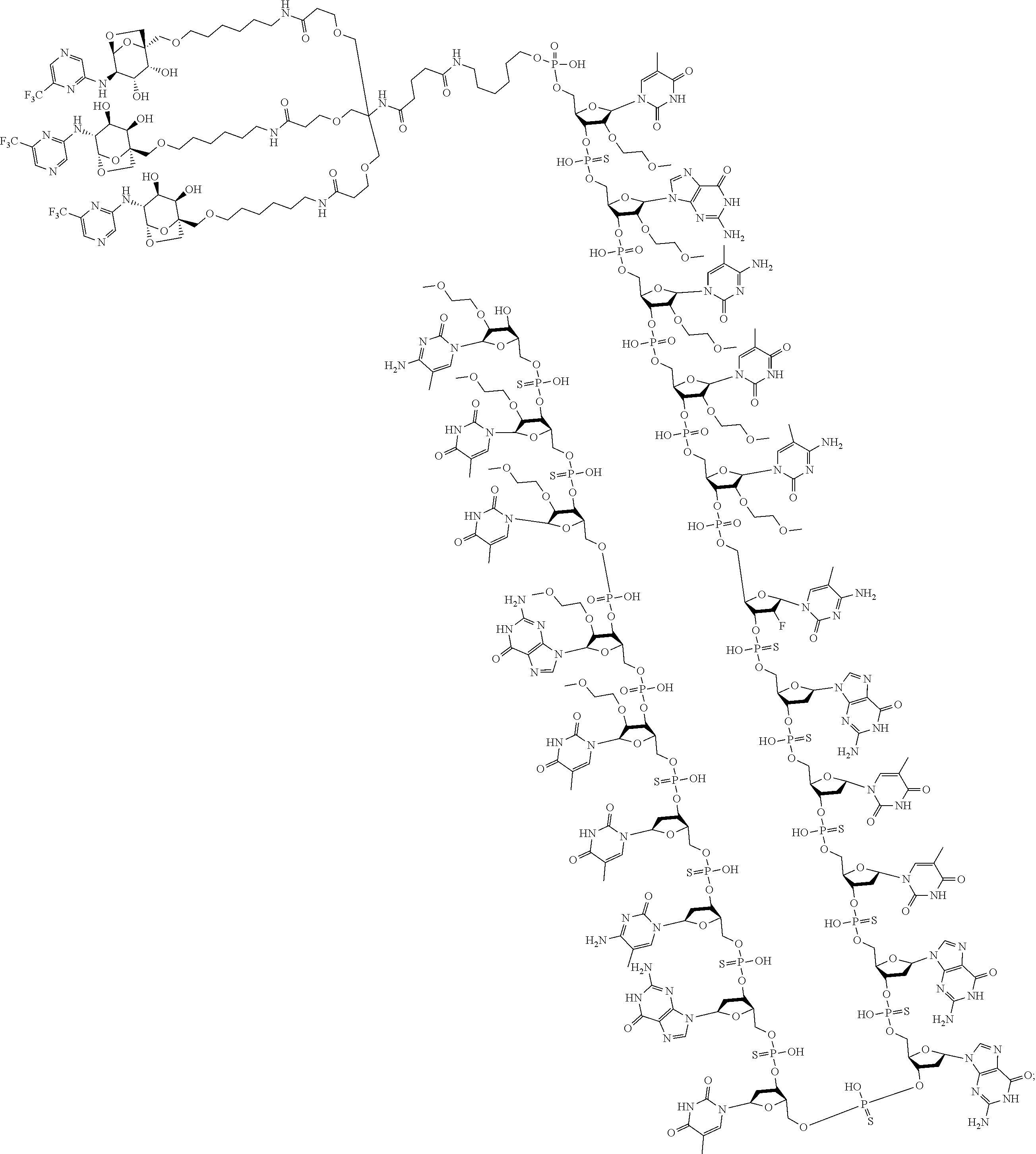

or a pharmaceutically acceptable salt thereof.

In certain embodiments, the Therapeutic Oligonucleotide delivering compound of the present invention is:

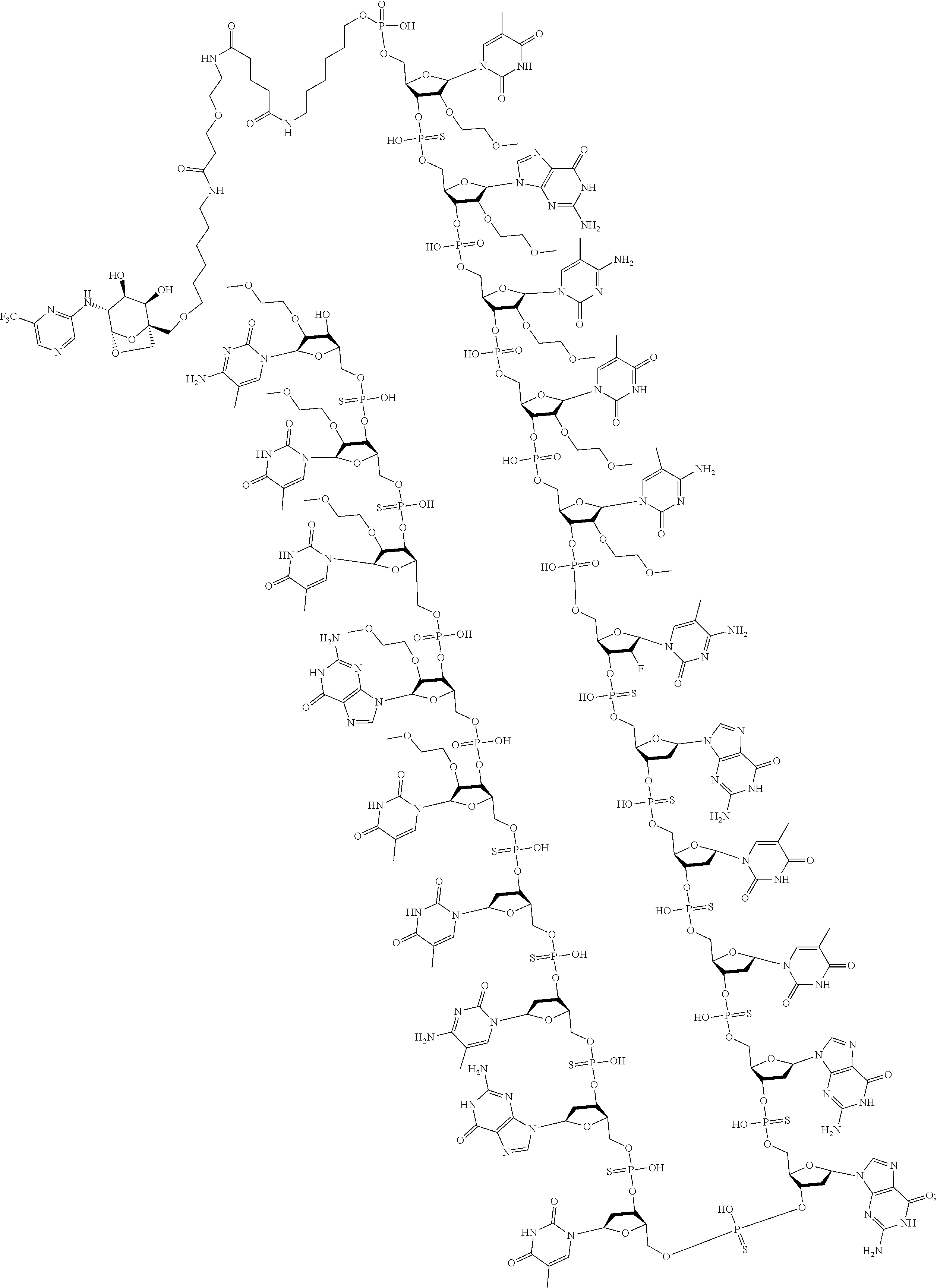

or a pharmaceutically acceptable salt thereof.

In certain embodiments, the Therapeutic Oligonucleotide delivering compound of the present invention is:

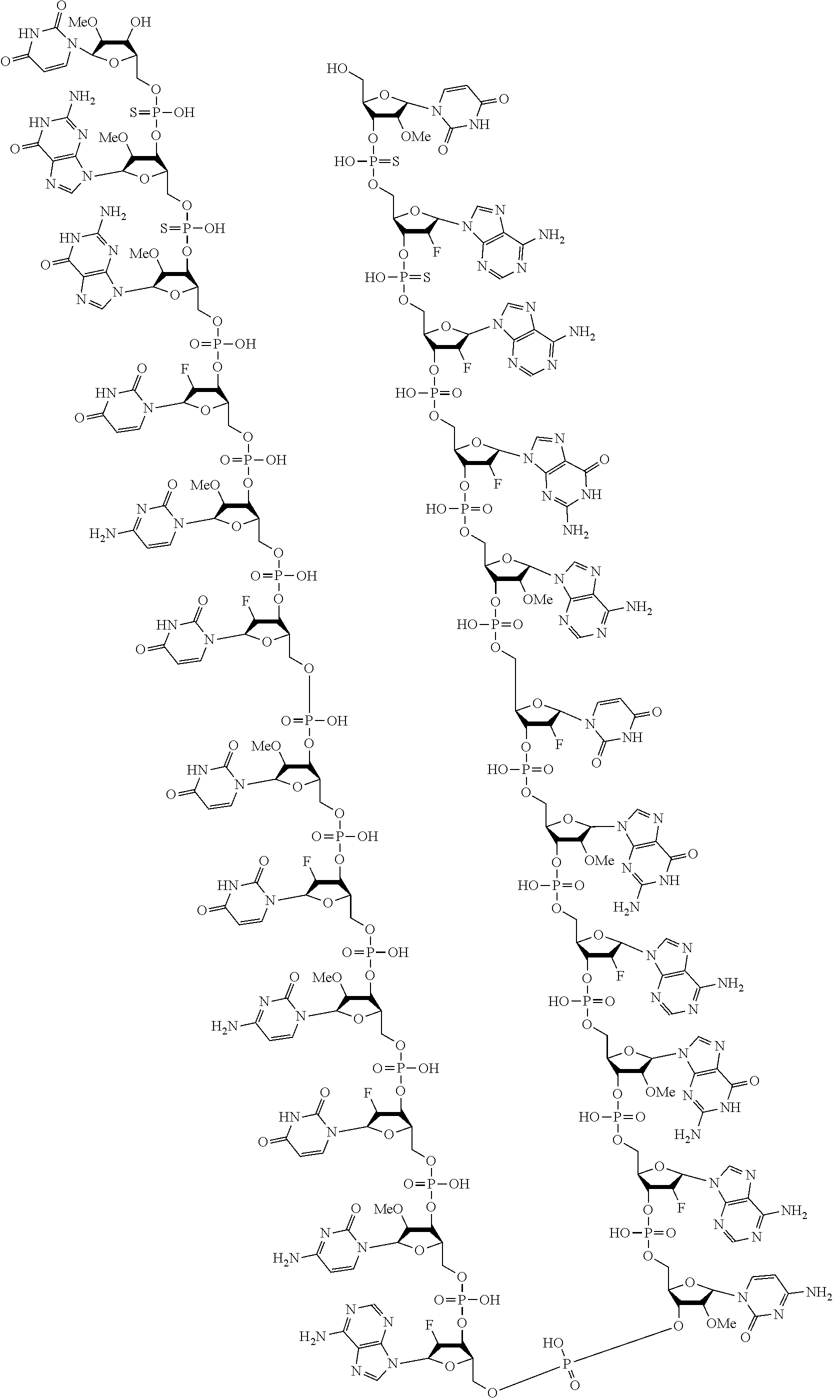

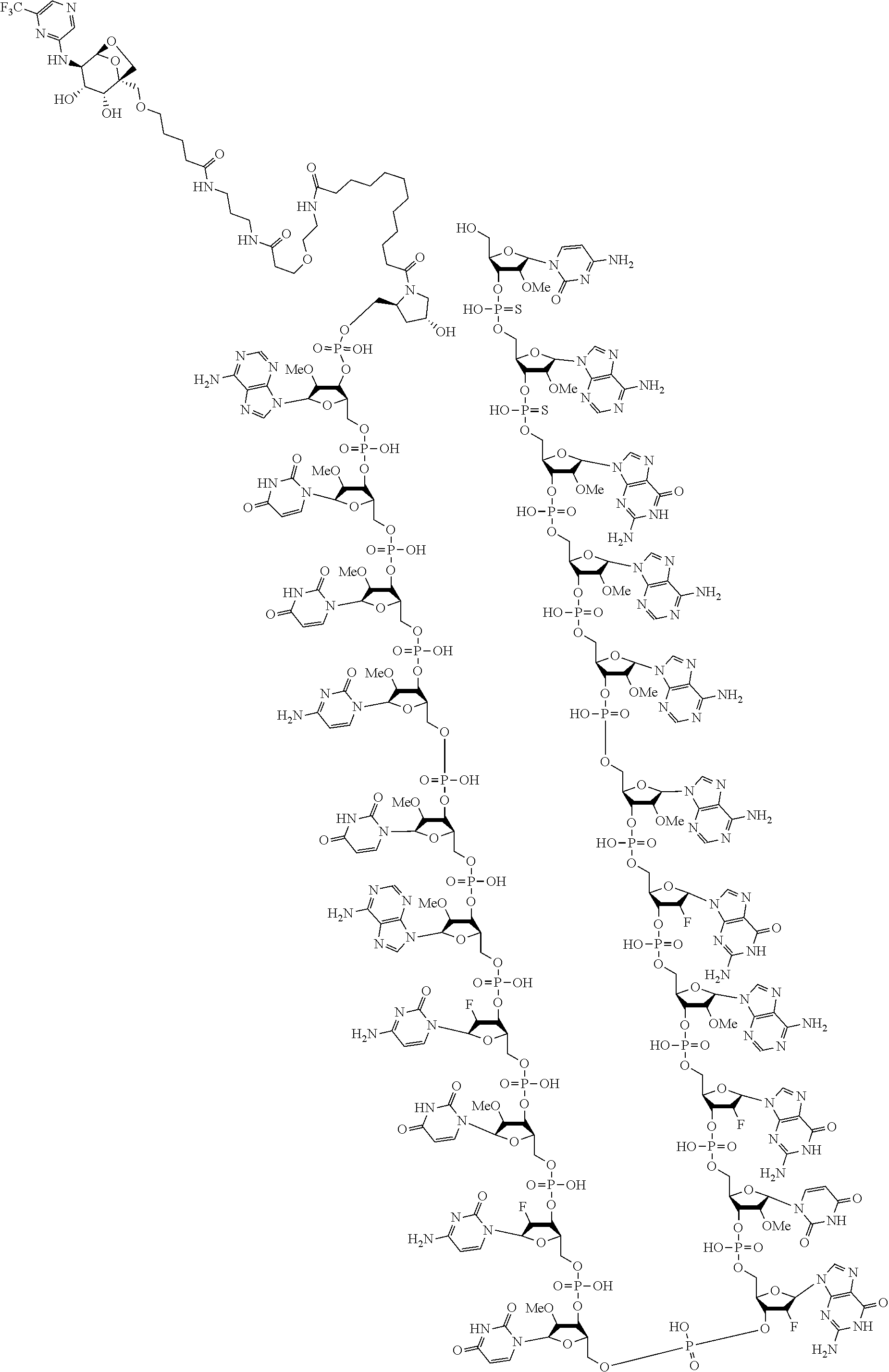

or a pharmaceutically acceptable salt thereof.

In certain embodiments, the Therapeutic Oligonucleotide delivering compound of the present invention is:

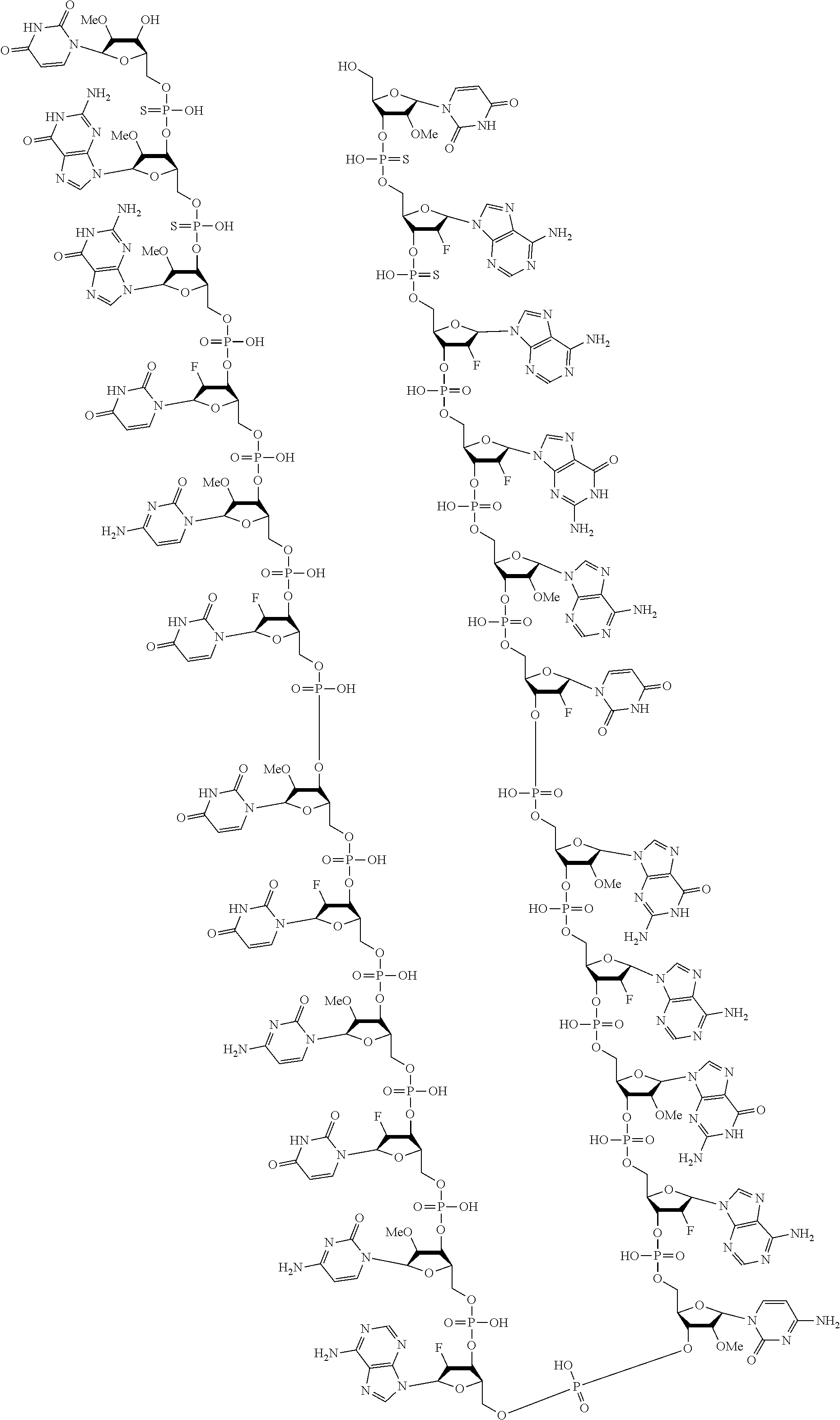

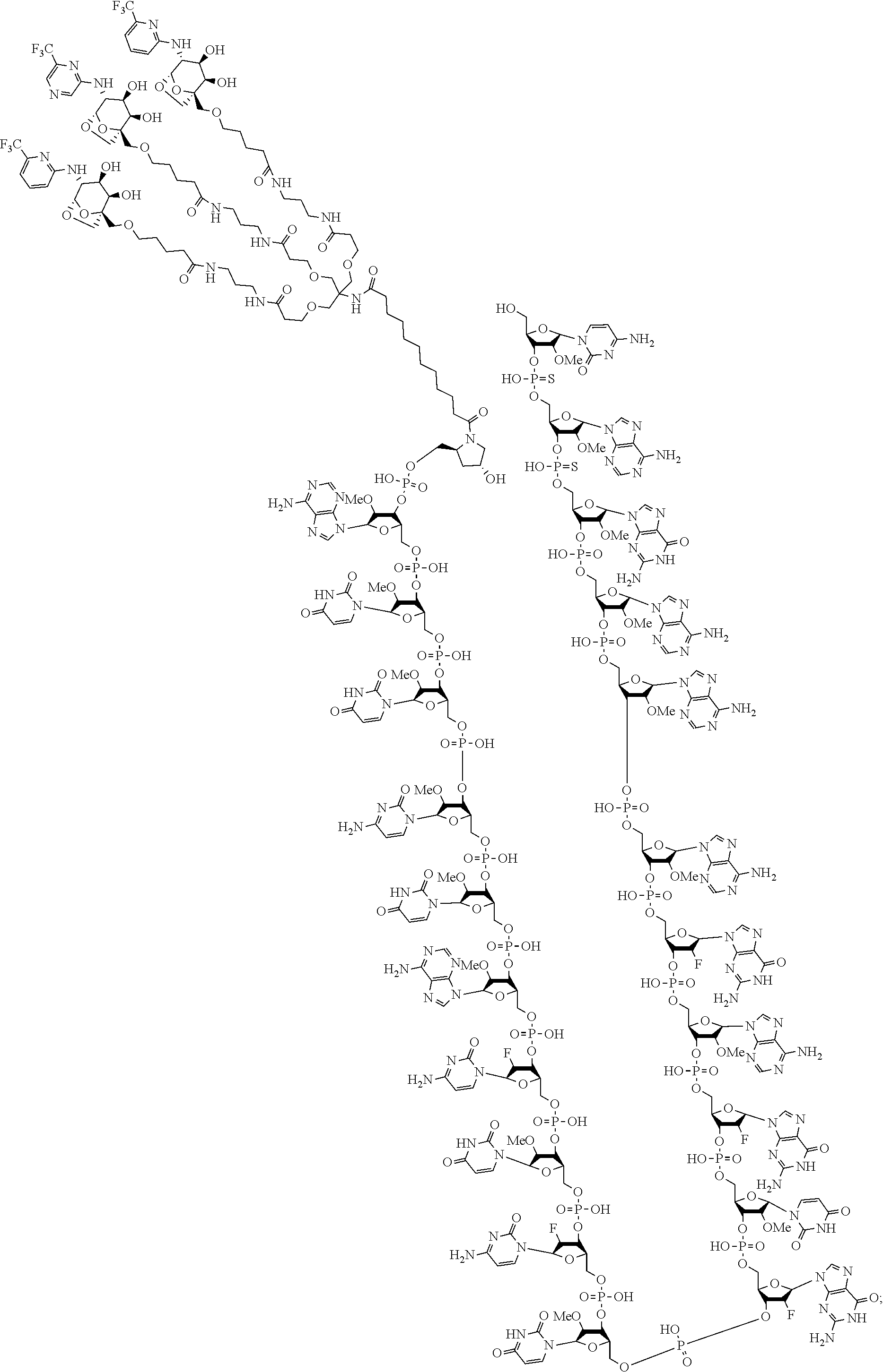

or a pharmaceutically acceptable salt thereof.

In certain embodiments, the Therapeutic Oligonucleotide delivering compound of the present invention is:

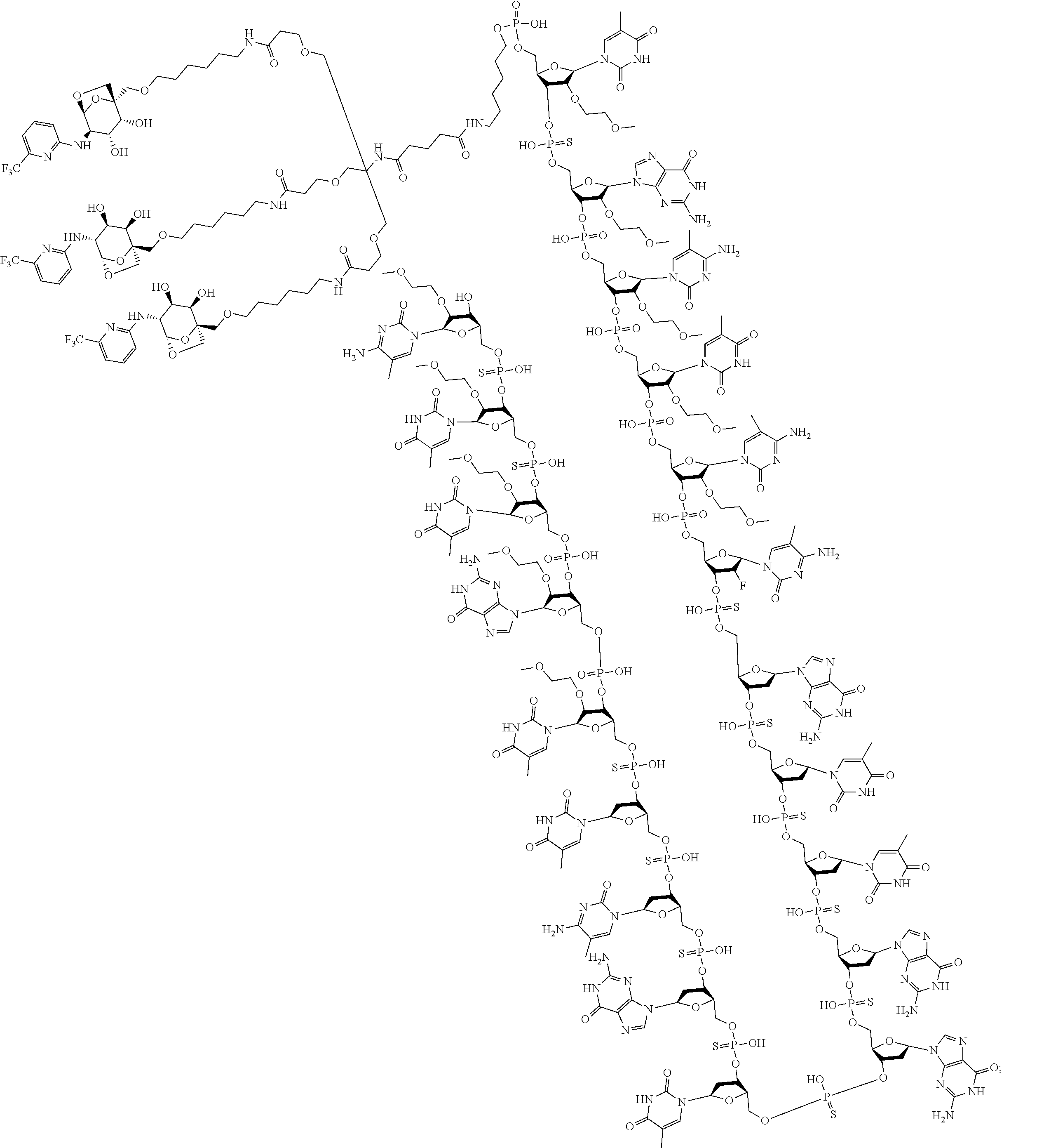

or a pharmaceutically acceptable salt thereof.

In certain embodiments, the Therapeutic Oligonucleotide delivering compound of the present invention is:

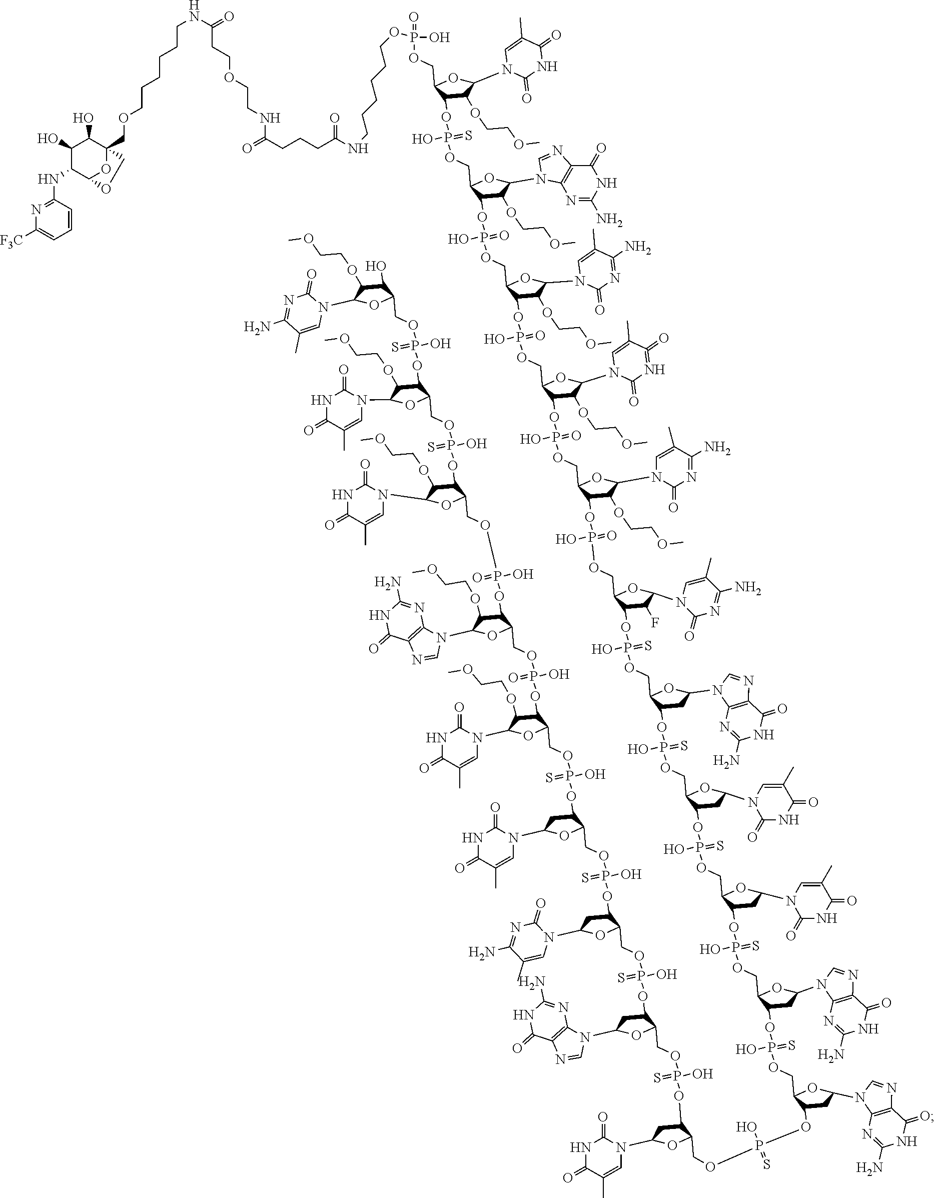

or a pharmaceutically acceptable salt thereof.

In certain embodiments, the Therapeutic Oligonucleotide delivering compound of the present invention is:

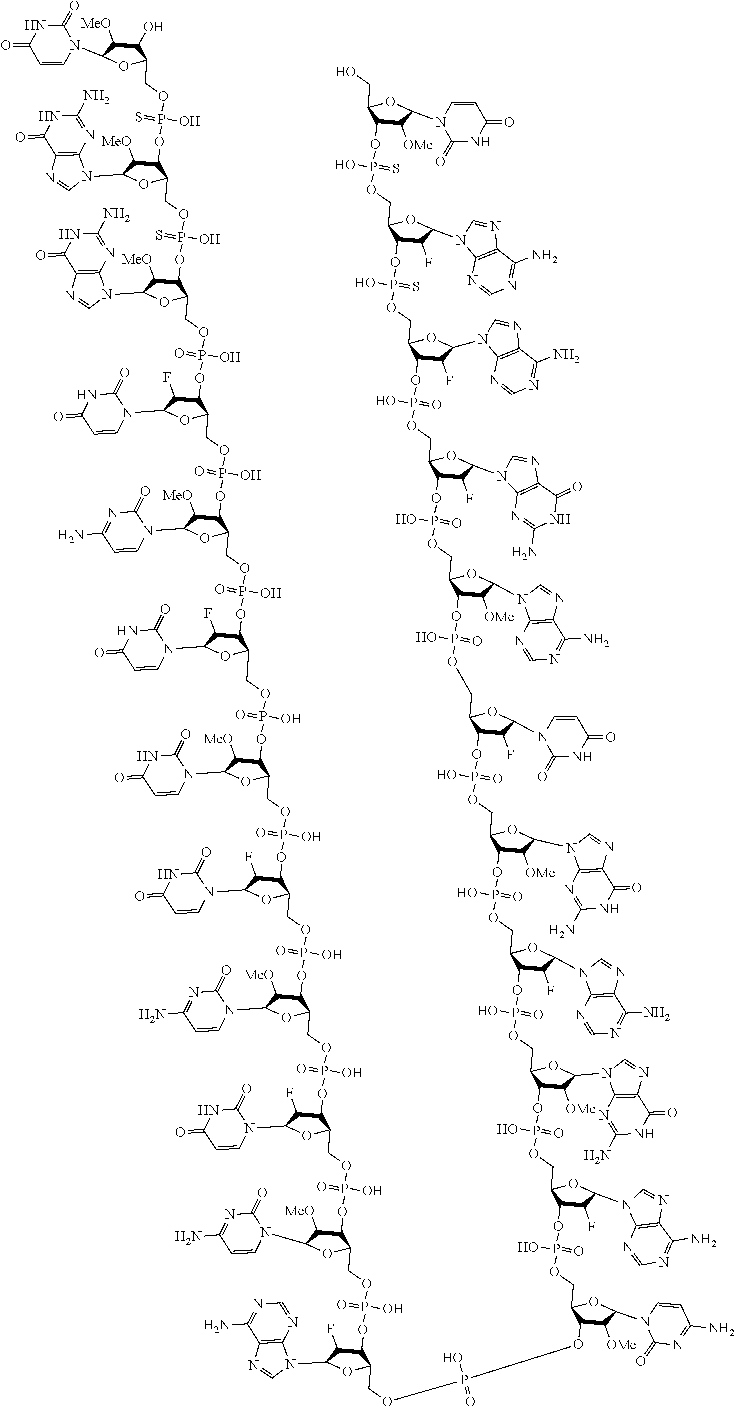

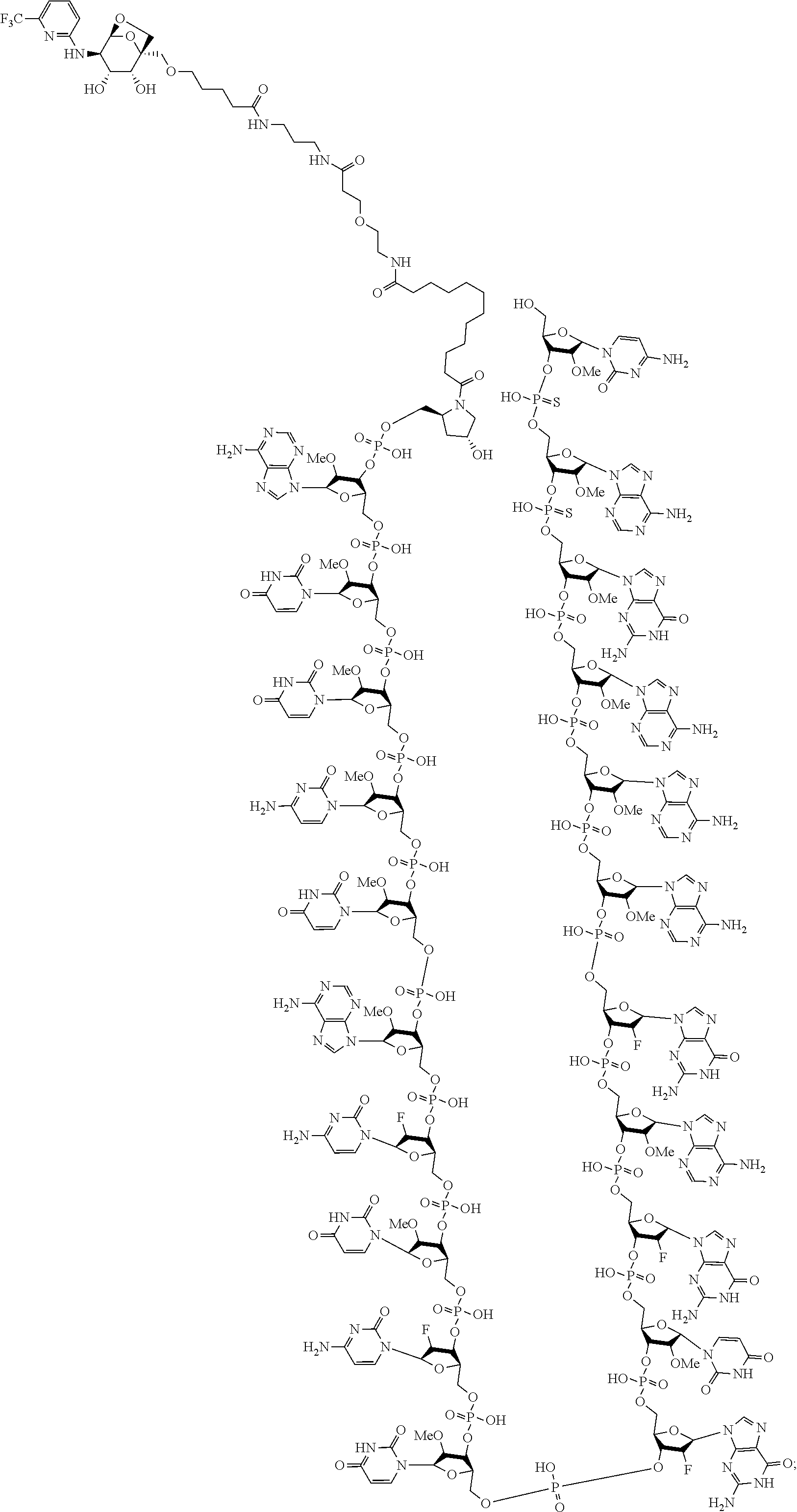

or a pharmaceutically acceptable salt thereof.

Examples of publications describing the use of ASGPR ligands for the delivery of oligonucleotides include: an article from Mina Therapeutics titled “Delivery of Oligonucleotides to the Liver with GalNAc: From Research to Registered Therapeutic Drugs” (Debacker, et al. Molecular Therapy (2022), 28; 8, 1759); an article from GlycoNovo Technologies Co. titled “Liver-Targeted Delivery of Oligonucleotides with N-Acetylgalactosamine Conjugation” (Cui et al. ACS Omega (2021), 6, 16259); WO 2014/179626 and US 2022/153775 assigned to Ionis Pharmaceuticals, Inc.; WO 2020/163747 assigned to Arrowhead Pharmaceuticals Inc.; WO 2022/098990 assigned to Arbutus Biopharma Corporation; and WO 2022/226217 assigned to Civi Biopharma, Inc. Additional examples of patents and patent applications describing therapeutic oligonucleotides include U.S. Pat. Nos. 8,106,022 8,450,467, 8,828,956, 9,352,048, 9,370,581, 9,370,582, 9,814,777, 9,828,606, 9,867,882, 10,125,369, and 10,131,907, assigned to Alnylam Pharmaceuticals, Inc.; and U.S. Pat. Nos. 9,150,605, 9,708,610, and 11,078,485 assigned to Ionis Pharmaceuticals, Inc.

Abbreviations have the following meanings:

| | |
|---|---|
| A | adenosine-3'-phosphate |
| C | cytidine-3'-phosphate |
| G | guanosine-3'-phosphate |
| U | uridine-3'-phosphate |
| Af | adenine 2'-F ribonucleoside |
| Cf | cytosine 2'F ribonucleoside |
| Uf | uracil 2'-F ribonucleoside |
| Am | adenine 2'-OMe ribonucleoside |
| Cm | cytosine 2'-OMe ribonucleoside |
| Gf | guanine 2'-F ribonucleoside |
| Gm | guanine 2'-OMe ribonucleoside |
| Um | uracil 2'-OMe ribonucleoside |
| n | any 2'-OMe modified nucleotide |
| a | 2'-O-methyladenosine-3'-phosphate |
| as | 2'-O-methyladenosine-3'-phosphorothioate |
| C | 2'-O-methylcytidine-3'-phosphate |
| CS | 2'-O-methylcytidine-3'-phosphorothioate |
| g | 2'-O-methylguanosine-3'-phosphate |
| gs | 2'-O-methylguanosine-3'-phosphorothioate |
| t | 2'-O-methyl-5-methyluridine-3'-phosphate |
| ts | 2'-O-methyl-5-methylmidine-3'-phosphorothioate |
| u | 2'-O-methyluridine-3'-phosphate |
| us | 2'-O-methyluridine-3'-phosphorothioate |
| Nf | any 2'-fluoro modified nucleotide |
| Af | 2'-fluoroadenosine-3'-phosphate |
| Afs | 2'-fluoroadenosine-3'-phosphorothioate |
| Cf | 2'-fluorocytidine-3'-phosphate |
| Cfs | 2'-fluorocytidine-3'-phosphorothioate |
| Gf | 2'-fluoroguanosine-3'-phosphate |
| Gfs | 2'-fluoroguanosine-3'-phosphorothioate |
| Tf | 2'-fluoro-5'-methyluridine-3'-phosphate |
| Tfs | 2'-fluoro-5'-methyluridine-3'-phosphorothioate |
| Uf | 2'-fluorouridine-3'-phosphate |
| Ufs | 2'-fluorouridine-3'-phosphorothioate |
| dN | any 2'-deoxyribonucleotide |
| dT | 2'-deoxythymidine-3'-phosphate |
| N_{UNA} | 2',3'-seco nucleotide mimics (unlocked nucleobase analogs) |
| N_{LNA} | locked nucleotide |
| Nf_{ANA} | 2'-F-Arabino nucleotide |
| NM | 2'-methoxyethyl nucleotide |
| AM | 2'-methoxyethyl adenosine-3'-phosphate |
| AMs | 2'-methoxyethyladenosine-3'-phosphorothioate |
| TM | 2'-methoxyethylthymidine-3'-phosphate |
| TMs | 2'-methoxyethylthymidine-3'-phosphorothioate |
| R | ribitol |
| (invdN) | any inverted deoxyribonucleotide (3'-3' linked nucleotide) |
| (invAb) | inverted (3'-3' linked) abasic deoxyribonucleotide, see Table 2 |
| (invAB)s | inverted (3'-3' linked) abasic deoxyribonucleotide- |
| | 5'-phosphorothioate, see Table 2 |
| (invn) | any inverted 2'-0:Me nucleotide (3'-3' linked nucleotide) |
| S | phosphorothioate linkage |
| vpdN | vinyl phosphonate deoxyribonucleotide |
| (5Me-Nf) | 5'-Me, 2'-fluoro nucleotide |
| cPrp | cyclopropyl phosphonate, see Table 2 |

TABLE 2
Exemplary modified nucleotides

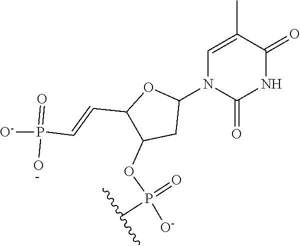 [SMILES: *P(=O)([O-])OC1CC(n2cc(C)c(=O)[nH]c2=O)OC1/C=C/P(=O)([O-])O]

vpdT

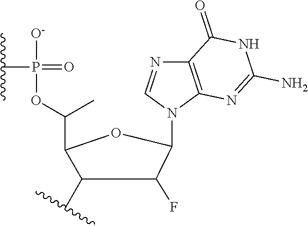 [SMILES: *C1C(F)C(n2cnc3c(=O)[nH]c(N)nc32)OC1C(C)OP(*)(=O)[O-]]

5Me-Gf

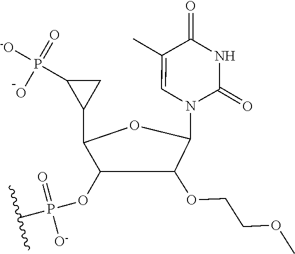

CPrpTM

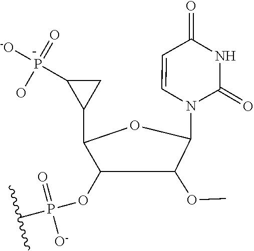

CPrpu

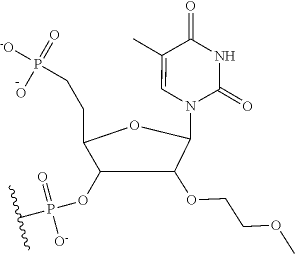

epTM

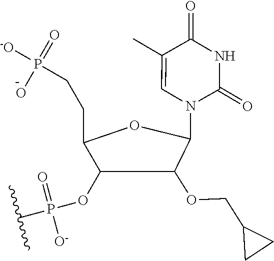

ePTcPr
When positioned internally on oligonucleotide:

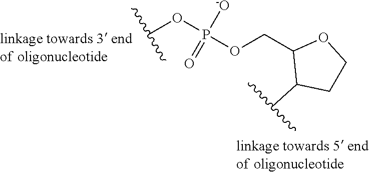

(invAb)
When positioned internally on oligonucleotide:

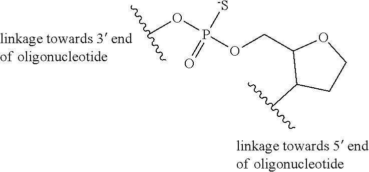

(invAb)s
When positioned at the 3′ terminal end of the oligonucleotide

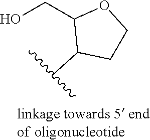 [SMILES: *C1CCOC1CO]

(invAb)

In certain embodiments the Therapeutic Oligonucleotide is a siRNA. A siRNA is a double-stranded RNA that can operate within the RNA interference pathway. RNA interference is a defense against viral infection used by many eukaryotic species. Once inside the cell, a siRNA can bind to a complementary mRNA sequence and induce degradation of that mRNA sequence. Reducing the amount of mRNA encoding a protein can result in reduced translation of the protein. In certain embodiments, the mRNA sequence that the siRNA can bind to encodes a protein related to a disease. In certain embodiments the siRNA is between 20 and 24 base pairs in length.

In certain embodiments the Therapeutic Oligonucleotide is a shRNA. A shRNA (short hairpin RNA) is an artificial RNA molecule with a tight hairpin turn that can be used to silence target gene expression via RNA interference. RNA interference using a shRNA molecule can lead to long-lasting reduction in protein expression.

In certain embodiments the Therapeutic Oligonucleotide is a DNA aptamer. A DNA aptamer is a single-stranded DNA molecule that can bind to a specific target with high affinity. A DNA aptamer can be produced through systematic evolution of ligands by exponential enrichment (SELEX), a form of combinatorial screening wherein aptamers that bind the desired target are subjected to subsequent rounds of optimization.

In certain embodiments the Therapeutic Oligonucleotide is an RNA aptamer. An RNA aptamer is a single-stranded RNA molecule that can bind to a specific target with high affinity.

In certain embodiments the Therapeutic Oligonucleotide is a miRNA. A miRNA (MicroRNA) is a small, highly conserved non-coding RNA molecule involved in the regulation of gene expression. MicroRNAs are typically transcribed by RNA polymerases II and III.

In certain embodiments the Therapeutic Oligonucleotide is a miRNA mimic. A miRNA mimic is a nonnatural double-stranded miRNA-like RNA fragment. The RNA fragment is typically designed to have its 5′-end bearing a partially complementary motif to the selected sequence in the 3′UTR unique to the target gene.

In certain embodiments the Therapeutic Oligonucleotide is a DNA decoy. A DNA decoy is a non-natural DNA probe that binds a target DNA-binding protein.

In certain embodiments the Therapeutic Oligonucleotide is an RNA decoy. A RNA decoy is a non-natural RNA probe that binds a target RNA-binding protein.

In certain embodiments the Therapeutic Oligonucleotide is a CpG oligonucleotide. A CpG oligonucleotide is a short single-stranded synthetic DNA molecule that contain a cytosine triphosphate deoxynucleotide.

Non-limiting examples of compounds of the present invention include:

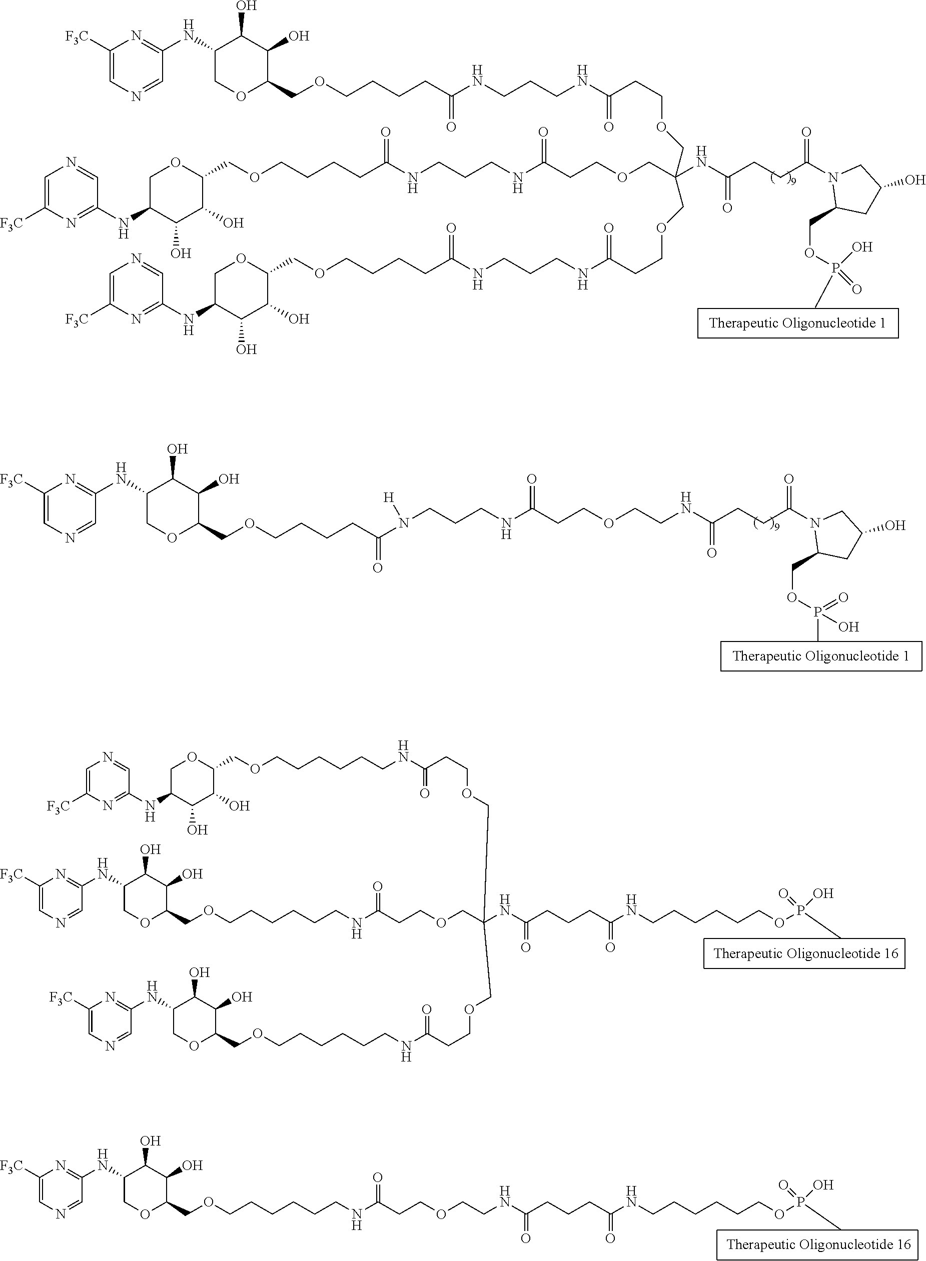

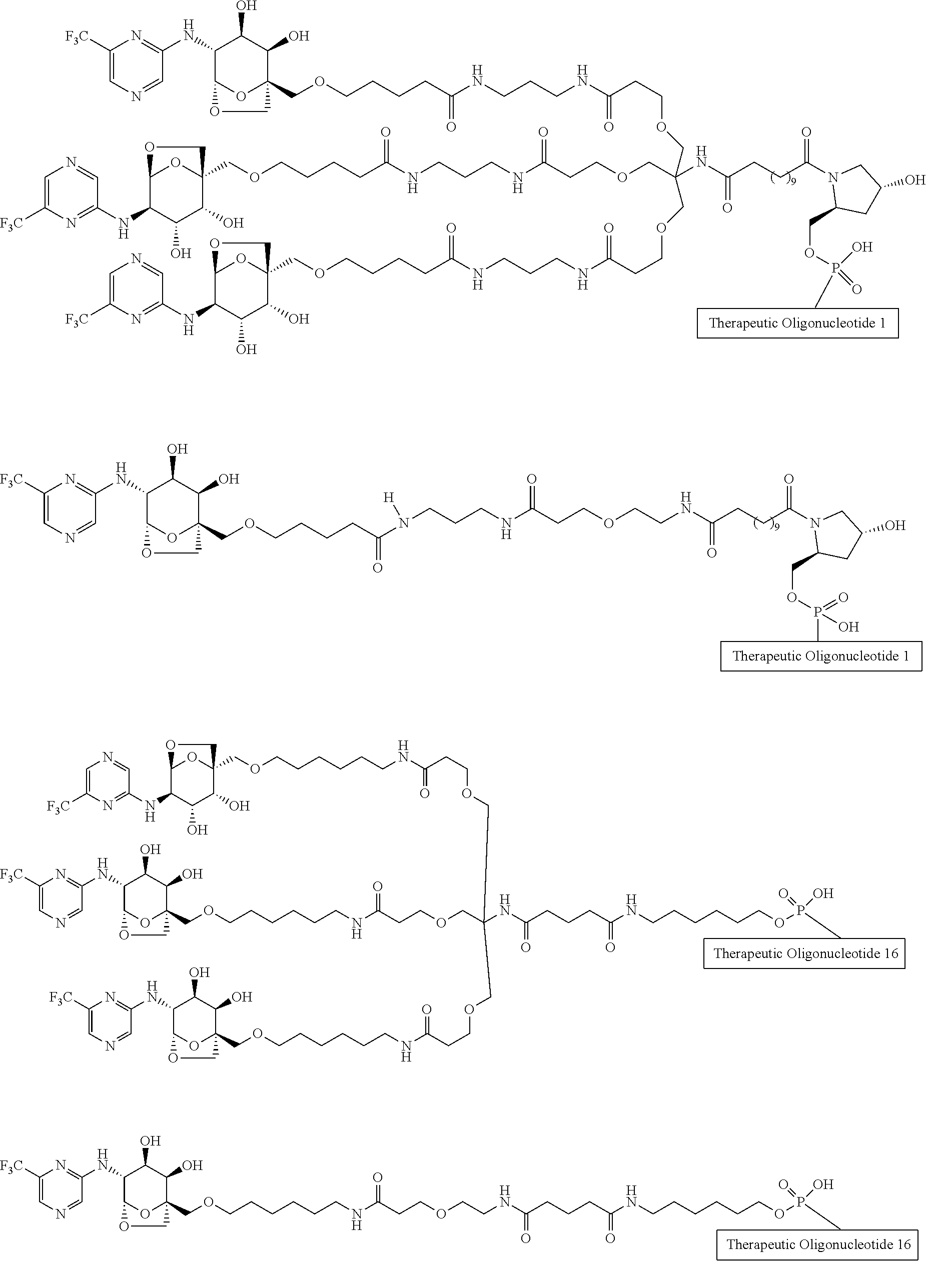

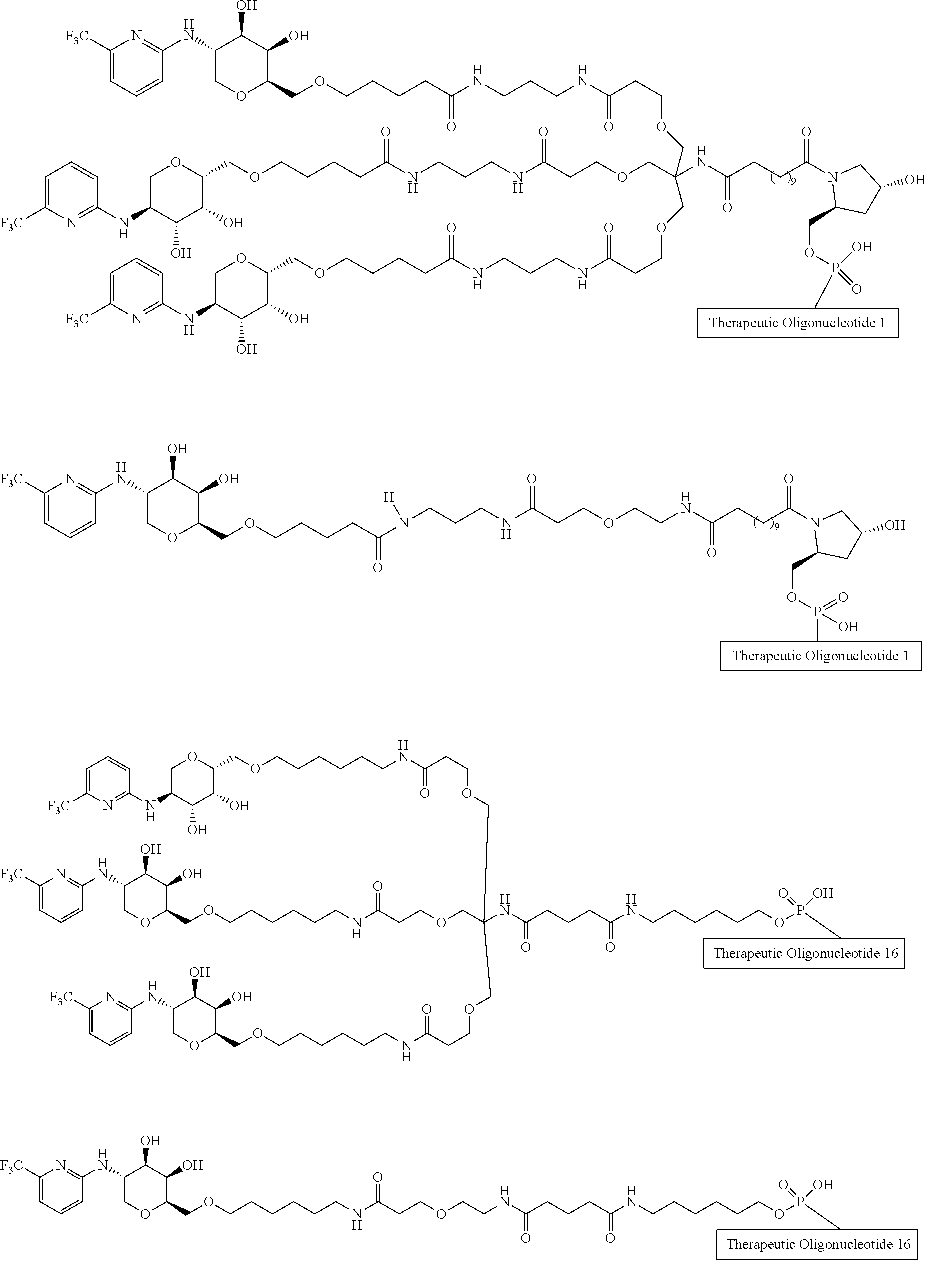

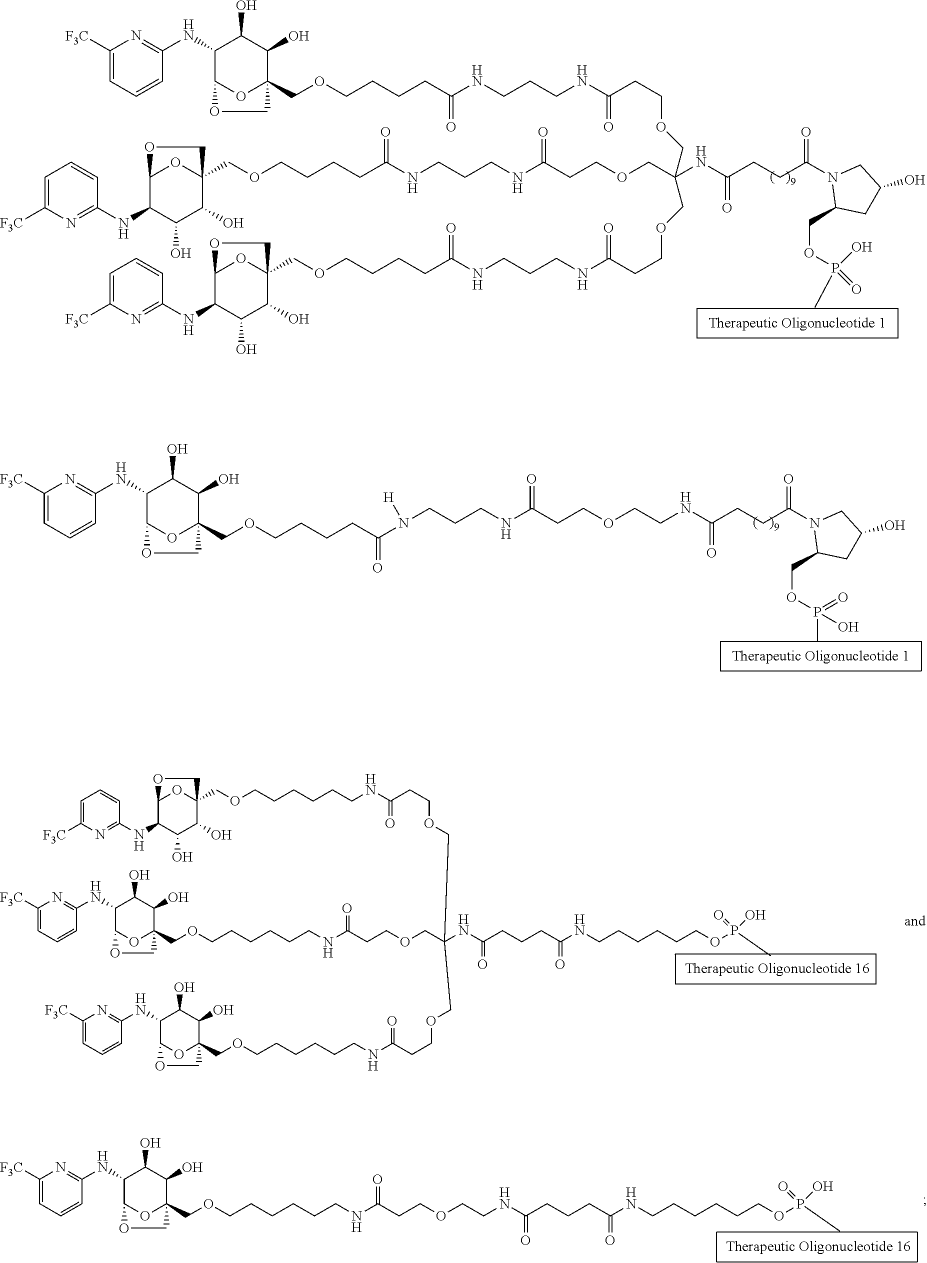

or a pharmaceutically acceptable salt thereof.

In certain embodiments, the compound of the present invention is selected from:

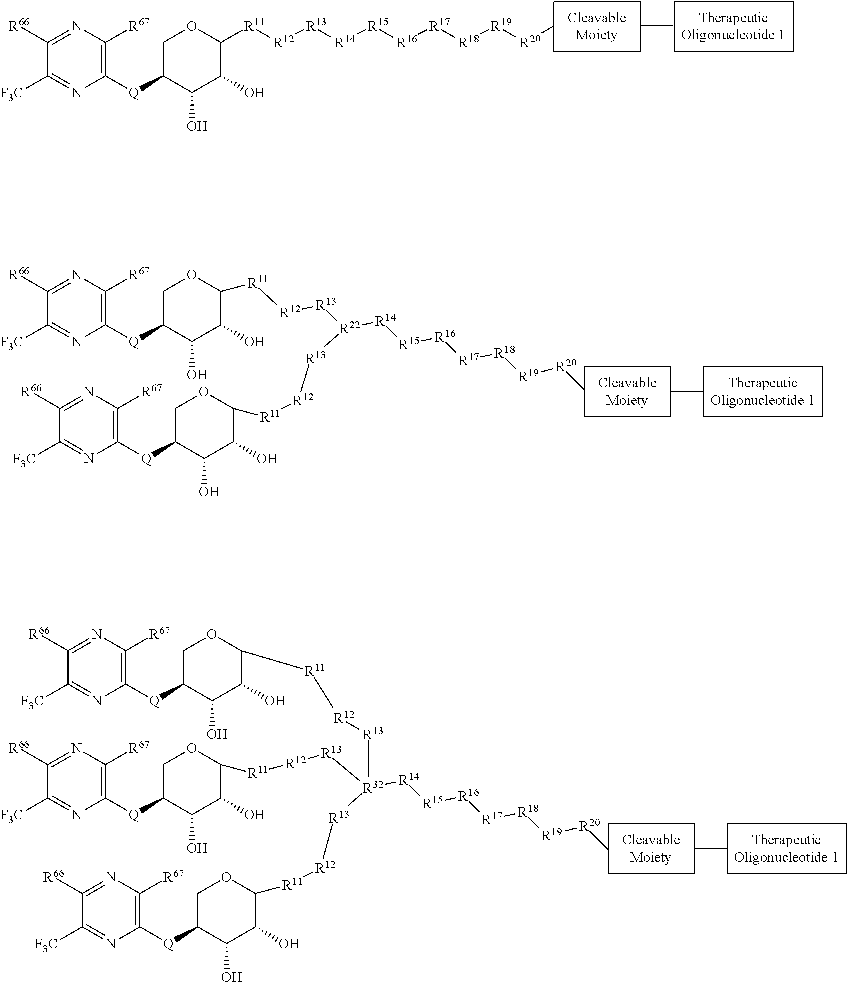

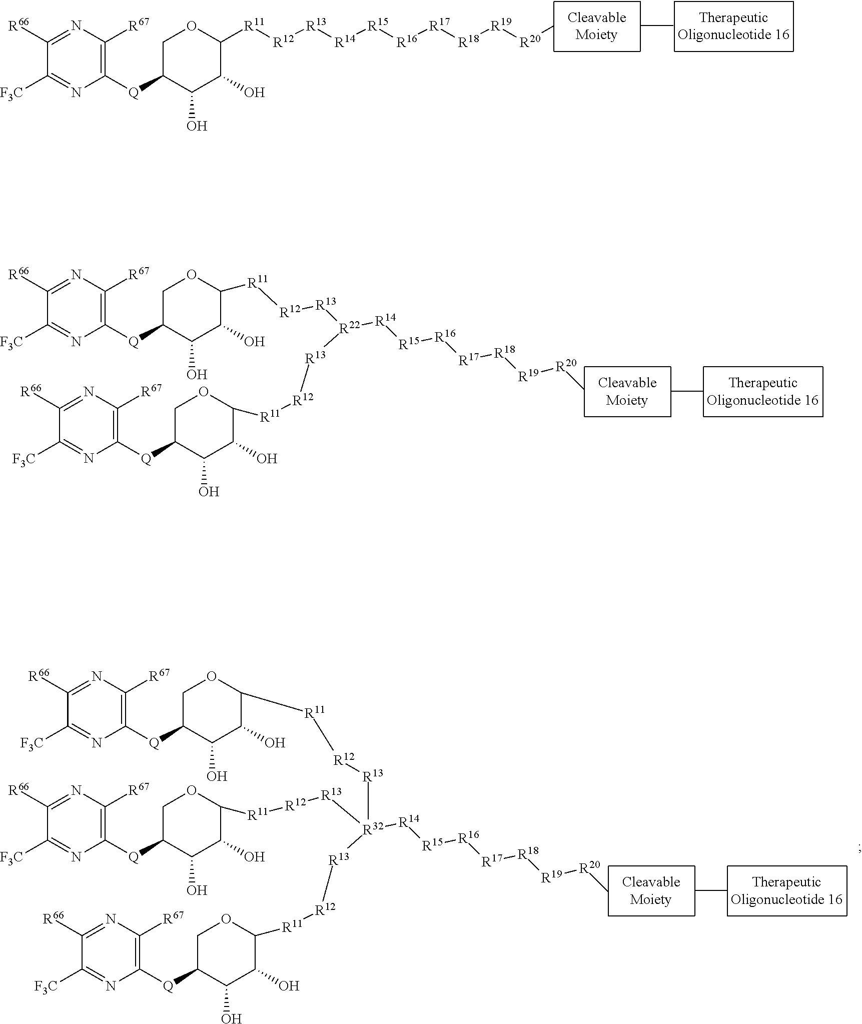

or a pharmaceutically acceptable salt thereof.

In certain embodiments, the compound of the present invention is selected from:

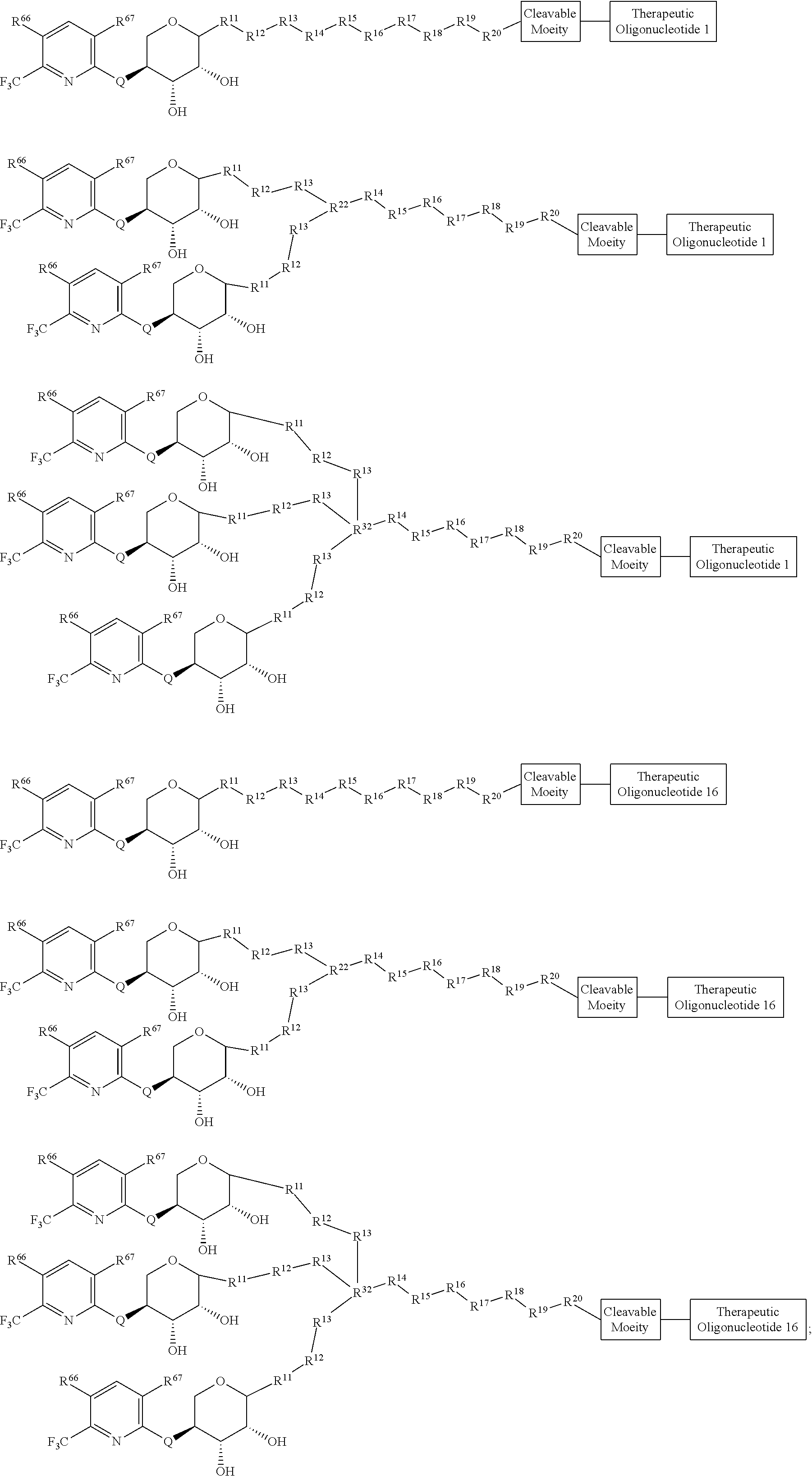

or a pharmaceutically acceptable salt thereof.

In certain embodiments, the compound of the present invention is selected from:

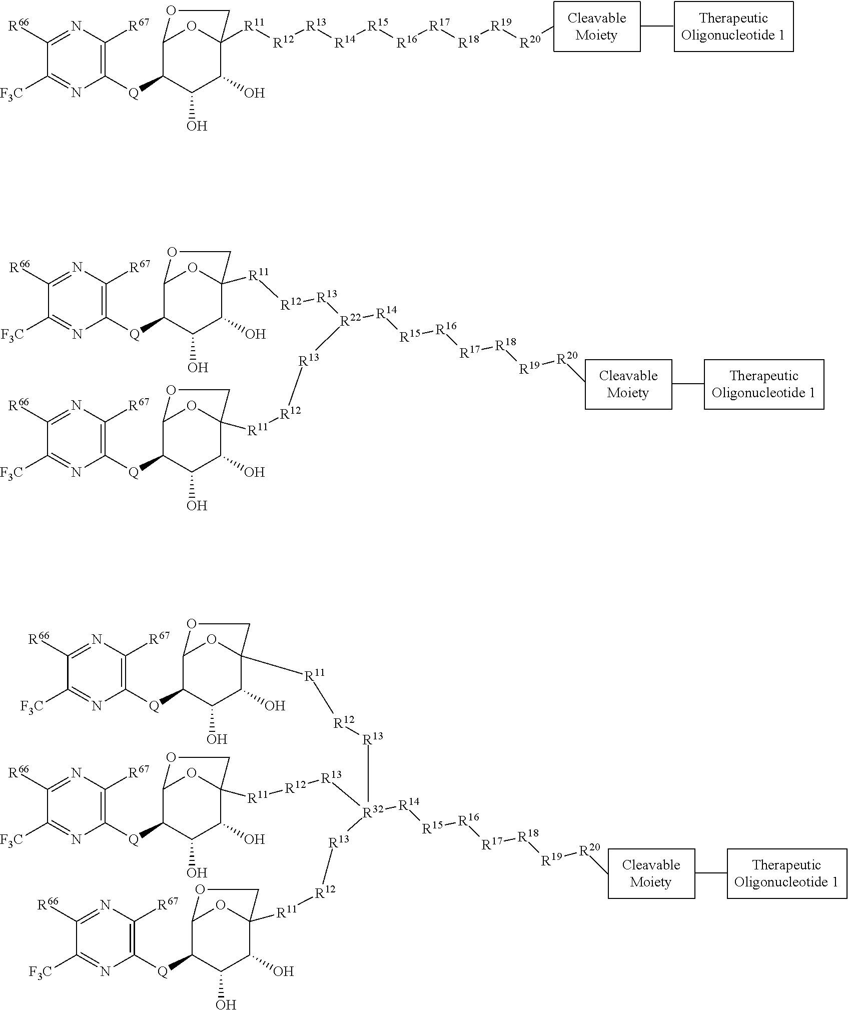

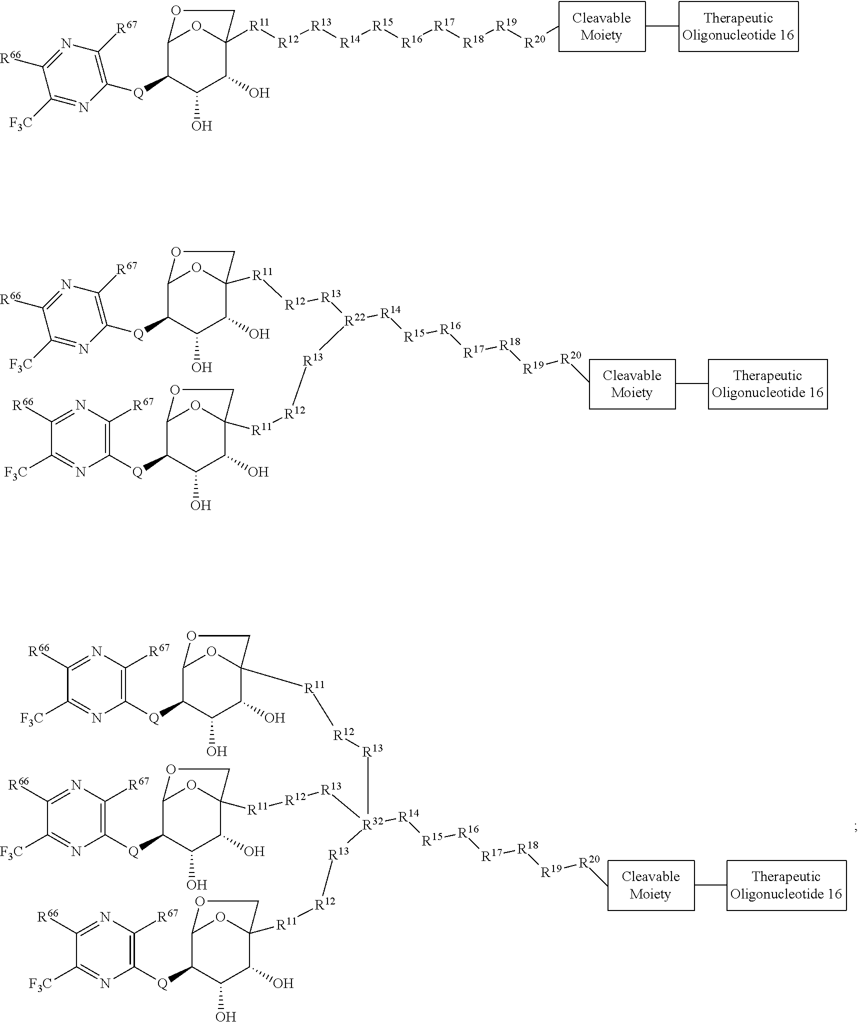

or a pharmaceutically acceptable salt thereof.

In certain embodiments, the compound of the present invention is selected from:

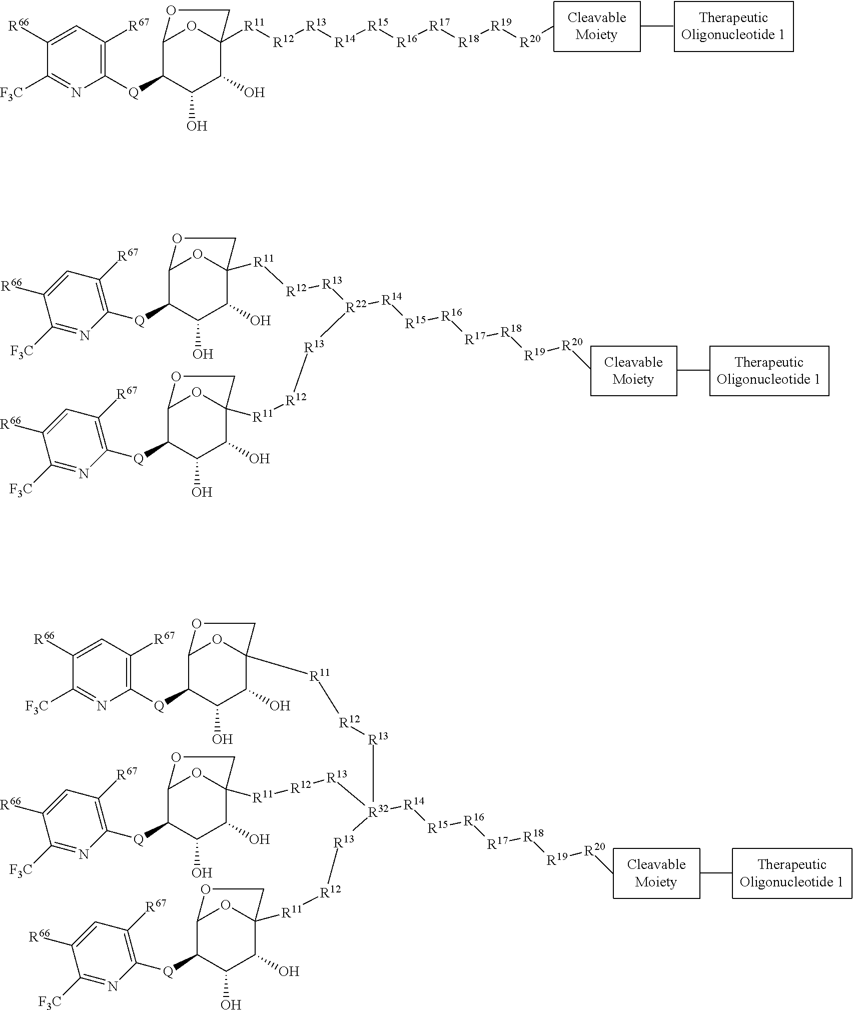

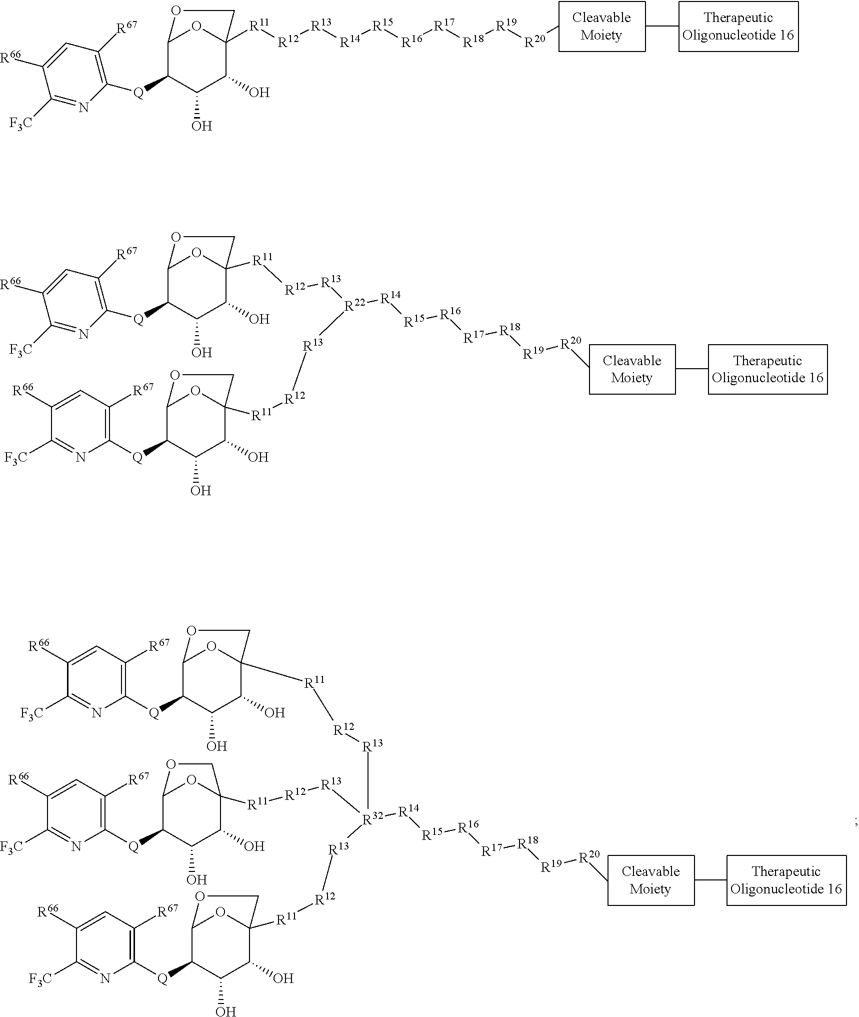

or a pharmaceutically acceptable salt thereof.

### III. Pharmaceutical Compositions and Dosage Forms for the Therapeutic Nucleotide Delivering Compounds of the Present INVENTION

A Therapeutic Oligonucleotide delivering compound of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder that can be treated with the Therapeutic Oligonucleotide.

In certain embodiments, the present invention provides pharmaceutical compositions comprising a Therapeutic Oligonucleotide delivering compound of the present invention or a pharmaceutically acceptable composition, salt, isotopic analog such as a deuterated derivative, or prodrug thereof, and a pharmaceutically acceptable excipient. In certain embodiments, the Therapeutic Oligonucleotide delivering compound is present in an effective amount, e.g., a therapeutically effective amount or a prophylactically effective amount.

The ASGPR-binding Therapeutic Oligonucleotide delivering compound of the present invention can be administered in any manner that allows the delivery of the Therapeutic Oligonucleotide. As such, examples of methods to deliver the compound of the present invention include, but are not limited to, oral, intravenous, sublingual, subcutaneous, parenteral, buccal, rectal, intra-aortal, intracranial, subdermal or transnasal, or by other means, in dosage unit formulations containing one or more conventional pharmaceutically acceptable carriers, as appropriate.

In certain embodiments the Therapeutic Oligonucleotide delivering compound of the present invention is administered orally. Typically, for oral administration the Therapeutic Oligonucleotide delivering compound will be formulated in a solid dosage form for oral administration or as a gel containing capsule. Non-limiting examples of solid dosage forms include capsules, tablets, and powders.

In certain embodiments the pharmaceutical composition comprises a caprylic acid derivative. Non-limiting examples of caprylic acid derivatives are provided in the section “CAPRYLIC ACID DERIVATIVES.” In certain aspects the pharmaceutical composition comprising a caprylic acid derivative is delivered orally.

In certain embodiments the Therapeutic Oligonucleotide delivering compound of the present invention is administered intravenously. For example, the Therapeutic Oligonucleotide delivering compound may be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

In certain embodiments the Therapeutic Oligonucleotide delivering compound of the present invention is administered subcutaneously. Typically, the Therapeutic Oligonucleotide delivering compound will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

Therefore, the disclosure provides pharmaceutical compositions comprising an effective amount of a Therapeutic Oligonucleotide delivering compound or its pharmaceutically acceptable salt together with at least one pharmaceutically acceptable carrier for any appropriate use thereof. The pharmaceutical composition may contain a Therapeutic Oligonucleotide delivering compound or salt as the only active agent, or, in an alternative embodiment, the Therapeutic Oligonucleotide delivering compound and at least one additional active agent.

In certain embodiments the term pharmaceutically acceptable salt refers to a salt of the described Therapeutic Oligonucleotide delivering compound which is, within the scope of sound medical judgment, suitable for administration to a host such as a human without undue toxicity, irritation, allergic response, and the like, commensurate with a reasonable benefit/risk ratio, and effective for its intended use. These salts can be prepared during the final isolation and purification of the Therapeutic Oligonucleotide delivering compound or by separately reacting the Therapeutic Oligonucleotide delivering compound in its free form with a suitable organic or inorganic acid and then isolating the salt thus formed. Basic compounds are capable of forming a wide variety of different salts with various inorganic and organic acids. Acid addition salts of the basic Therapeutic Oligonucleotide delivering compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic Therapeutic Oligonucleotide delivering compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19.

Pharmaceutically acceptable excipients include inert diluents, dispersing and/or granulating agents, surface active agents and/or emulsifiers, disintegrating agents, binding agents, preservatives, buffering agents, lubricating agents, and/or oils. Additional acceptable excipients include cocoa butter and suppository waxes, coloring agents, coating agents, sweetening, flavoring, perfuming agents, etc., and combinations thereof.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum 32 silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, etc., and/or combinations thereof.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Any dosage form can be used that achieves the desired results. In certain embodiments the pharmaceutical composition is in a dosage form that contains from about 0.1 mg to about 1500 mg, from about 1 mg to about 1000 mg, from about 5 mg to about 1000 mg, from about 10 mg to about 1000 mg, from about 1 mg to about 500 mg, from about 5 mg to about 500 mg, from about 10 mg to about 500 mg, from about 1 mg to about 250 mg, from about 5 mg to about 250 mg, from about 10 mg to about 250 mg, from about 25 mg to about 800 mg, from about 50 mg to about 800 mg, from about 75 mg to about 800 mg, from about 100 mg to about 800 mg, or from about 100 mg to about 600 mg of the Therapeutic Oligonucleotide delivering compound and optionally from about 0.1 mg to about 1500 mg, from about 1 mg to about 1000 mg, from about 5 mg to about 1000 mg, from about 10 mg to about 1000 mg, from about 1 mg to about 500 mg, from about 5 mg to about 500 mg, from about 10 mg to about 500 mg, from about 1 mg to about 250 mg, from about 5 mg to about 250 mg, from about 10 mg to about 250 mg, from about 25 mg to about 800 mg, from about 50 mg to about 800 mg, from about 75 mg to about 800 mg, from about 100 mg to about 800 mg, or from about 100 mg to about 600 mg of an additional active agent in a unit dosage form. Examples are dosage forms with at least about 0.1, 1, 5, 10, 25, 50, 100, 200, 250, 300, 400, 500, 600, 700, 750, 800, 850, 900, 950, or 1,000 mg of the Therapeutic Oligonucleotide delivering compound, or its salt. In certain embodiments the dosage form has at most about 0.1, 1, 5, 10, 25, 50, 100, 200, 250, 300, 400, 500, 600, 700, 750, 800, 850, 900, 950, or 1,000 mg of the Therapeutic Oligonucleotide delivering compound, or its salt.

In alternative embodiments, the pharmaceutical composition comprises from about 50 mg/mL to about 300 mg/mL, from about 100 mg/mL to about 250 mg/mL or from about 150 mg/mL to about 200 mg/mL of the Therapeutic Oligonucleotide delivering compound, or its salt. In certain embodiments, from about 0.5 mL to about 5 mL, from about 1 mL to about 2 mL or about 1.0, 1.2, 1.4, 1.6, 1.8, or 2.0 mL of the pharmaceutical composition comprising the Therapeutic Oligonucleotide delivering compound, or its salt, is administered. In certain embodiments, from about 0.5 mL to about 1.5 mL of the pharmaceutical composition comprising the Therapeutic Oligonucleotide delivering compound, or its salt, is administered subcutaneously. In certain embodiments, from about 1 mL to about 5 mL of the pharmaceutical composition comprising the Therapeutic Oligonucleotide delivering compound, or its salt, is administered subcutaneously in one, two, three, four, or more injections. In certain embodiments, if a patient is administered multiple subcutaneous injections to reach a total dose of about 1 mL to about 5 mL, the multiple subcutaneous injection sites are spaced apart by at least about 5 cm.

In alternative embodiments, the dose is from about 20 to about 300 mg, from about 50 mg to about 250 mg, or from about 100 mg to about 200 mg of the Therapeutic Oligonucleotide delivering compound, or its salt.

In certain embodiments the dose ranges from about 0.01-100 mg/kg of patient bodyweight, for example at least about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, the Therapeutic Oligonucleotide delivering compound disclosed herein or used as described are administered once a day (QD), twice a day (BID), or three times a day (TID). In some embodiments, the Therapeutic Oligonucleotide delivering compound disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In some embodiments, the Therapeutic Oligonucleotide delivering compound disclosed herein or used as described are administered once every two days, three, days, four days, five days, six days, or once every week. In some embodiments, the Therapeutic Oligonucleotide delivering compound disclosed herein or used as described are administered once every two weeks, three weeks, or once a month. In some embodiments, the Therapeutic Oligonucleotide delivering compound disclosed herein or used as described are administered once every two, three, four, five, six, seven, eight, nine, ten, eleven months or once per year.

In alternative embodiments, from about 20 to about 300 mg, from about 50 mg to about 250 mg, or from about 100 mg to about 200 mg of the Therapeutic Oligonucleotide delivering compound, or its salt, is administered once per month. In certain embodiments, from about 50 mg to about 250 mg of the pharmaceutical composition comprising the Therapeutic Oligonucleotide delivering compound, or its salt, is administered subcutaneously once per month. In certain embodiments, from about 50 mg to about 250 mg of the pharmaceutical composition comprising the Therapeutic Oligonucleotide delivering compound, or its salt, is administered subcutaneously in one, two, three, four, or more injections which are administered once per month.

In certain embodiments, the Therapeutic Oligonucleotide delivering compound of the present invention is administered at least once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device. Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert, or it can possess pharmaceutical benefits of its own. The amount of carrier employed in conjunction with the Therapeutic Oligonucleotide delivering compound is sufficient to provide a practical quantity of material for administration per unit dose of the Therapeutic Oligonucleotide delivering compound. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the Therapeutic Oligonucleotide delivering compound of the present invention.

The pharmaceutical compositions/combinations can be formulated for oral administration. These compositions can contain any amount of the Therapeutic Oligonucleotide delivering compound that achieves the desired result, for example between 0.1 and 99 weight % (wt. %) of the Therapeutic Oligonucleotide delivering compound and usually at least about 5 wt. % of the Therapeutic Oligonucleotide delivering compound. Some embodiments contain from about 25 wt. % to about 50 wt. % or from about 5 wt. % to about 75 wt. % of the Therapeutic Oligonucleotide delivering compound. Enteric coated oral tablets may also be used to enhance bioavailability of the Therapeutic Oligonucleotide delivering compound for an oral route of administration.

Formulations suitable for rectal administration are typically presented as unit dose suppositories. These may be prepared by admixing the Therapeutic Oligonucleotide delivering compound with one or more conventional solid carriers, for example, cocoa butter, and then shaping the resulting mixture.

Therapeutic Oligonucleotide delivering compounds of the present invention and pharmaceutically acceptable composition, salts, isotopic analogs, or prodrugs thereof, may be formulated in dosage unit form for ease of administration and uniformity of dosage. It will be understood, however, that the total daily usage of the compositions comprising a Therapeutic Oligonucleotide delivering compound as described herein will be decided by the attending physician within the scope of sound medical judgment. The specific therapeutically effective dose level for any particular subject or organism will depend upon a variety of factors including the disease, disorder, or condition being treated and the severity of the disorder; the activity of the specific the Therapeutic Oligonucleotide delivering compound employed; the specific composition employed; the age, body weight, general health, sex and diet of the subject; the time of administration, route of administration, and rate of excretion of the Therapeutic Oligonucleotide delivering compound employed; the duration of the treatment; drugs used in combination or coincidental with the Therapeutic Oligonucleotide delivering compound employed; and like factors well known in the medical arts.

The Therapeutic Oligonucleotide delivering compound and compositions provided herein can be administered by any route, including enteral (e.g., oral), parenteral, systemic, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), the condition of the subject (e.g., whether the subject is able to tolerate oral administration).

The exact amount of a Therapeutic Oligonucleotide delivering compound required to achieve an effective amount will vary from subject to subject, depending, for example, on species, age, and general condition of a subject, severity of the side effects or disorder, identity of the particular Therapeutic Oligonucleotide delivering compound(s), mode of administration, and the like. The desired dosage can be delivered using any frequency determined to be useful by the health care provider, including three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be also appreciated that a Therapeutic Oligonucleotide delivering compound or composition, as described herein, can be administered in combination with one or more additional therapeutically active agents. The Therapeutic Oligonucleotide delivering compound or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder (for example, a Therapeutic Oligonucleotide delivering compound can be administered in combination with an anti-inflammatory agent, anti-cancer agent, immunosuppressant, etc.), and/or it may achieve different effects (e.g., control of adverse side-effects, e.g., emesis controlled by an antiemetic).

The Therapeutic Oligonucleotide delivering compound or composition can be administered concurrently with, prior to, or subsequent to, one or more additional therapeutically active agents. In general, each agent will be administered at a dose and/or on a time schedule determined for that agent. It will further be appreciated that the additional therapeutically active agent used in this combination can be administered together in a single composition or administered separately in different compositions. The particular combination to employ in a regimen will take into account compatibility of the Therapeutic Oligonucleotide delivering compound with the additional therapeutically active agent and/or the desired therapeutic effect to be achieved. In general, it is expected that additional therapeutically active agents used in combination be used at levels that do not exceed the levels at which they are used individually. In some embodiments, the levels used in combination will be lower than those used individually.

Exemplary additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically active agent is an anti-cancer agent, e.g., radiation therapy and/or one or more chemotherapeutic agents.

In certain aspects, a treatment regimen is provided comprising the administration of a Therapeutic Oligonucleotide delivering compound of the present invention or a pharmaceutically acceptable composition, salt, isotopic analog (such as a deuterated derivative), or prodrug thereof in combination or in alternation with at least one additional therapeutic agent. The combinations and/or alternations can be administered for beneficial, additive, or synergistic effect in the treatment of a disorder which is treatable by the Therapeutic Oligonucleotide.

### IV. ASGPR Binding Ligands

In certain embodiments the ASGPR Binding Ligand is selected from:

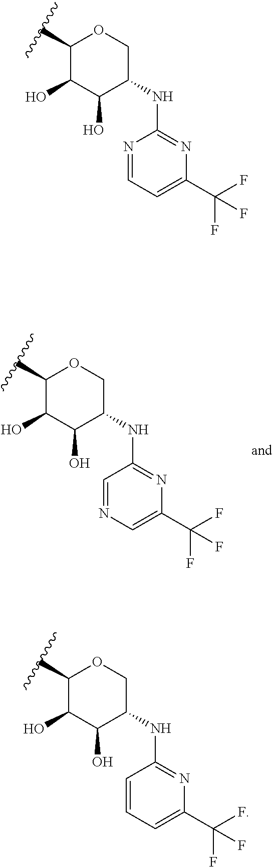 [SMILES: *[C@H]1OC[C@H](Nc2nccc(C(F)(F)F)n2)[C@@H](O)[C@H]1O ; *[C@H]1OC[C@H](Nc2cncc(C(F)(F)F)n2)[C@@H](O)[C@H]1O ; *[C@H]1OC[C@H](Nc2cccc(C(F)(F)F)n2)[C@@H](O)[C@H]1O]

In certain embodiments the ASGPR Binding Ligand is selected from:

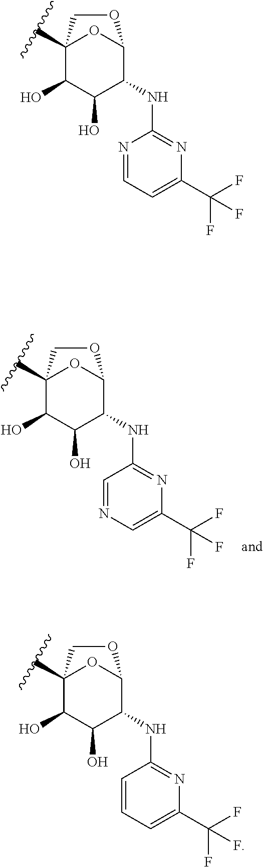 [SMILES: *[C@@]12CO[C@@H](O1)[C@H](Nc1nccc(C(F)(F)F)n1)[C@@H](O)[C@H]2O ; *[C@@]12CO[C@@H](O1)[C@H](Nc1cncc(C(F)(F)F)n1)[C@@H](O)[C@H]2O ; *[C@@]12CO[C@@H](O1)[C@H](Nc1cccc(C(F)(F)F)n1)[C@@H](O)[C@H]2O]

In certain embodiments, the ASGPR Binding Ligand is selected from

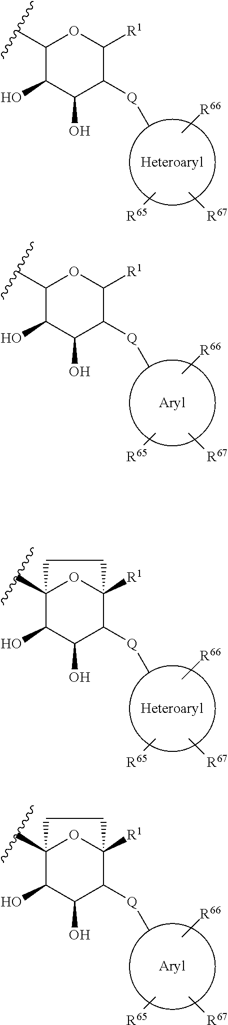

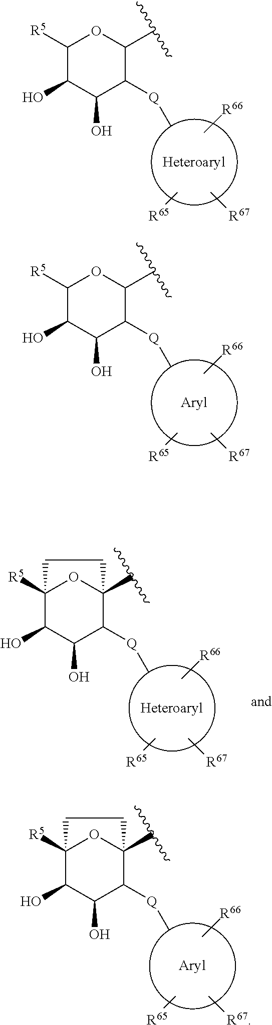

In certain embodiments the ASGPR Binding Ligand is selected from:

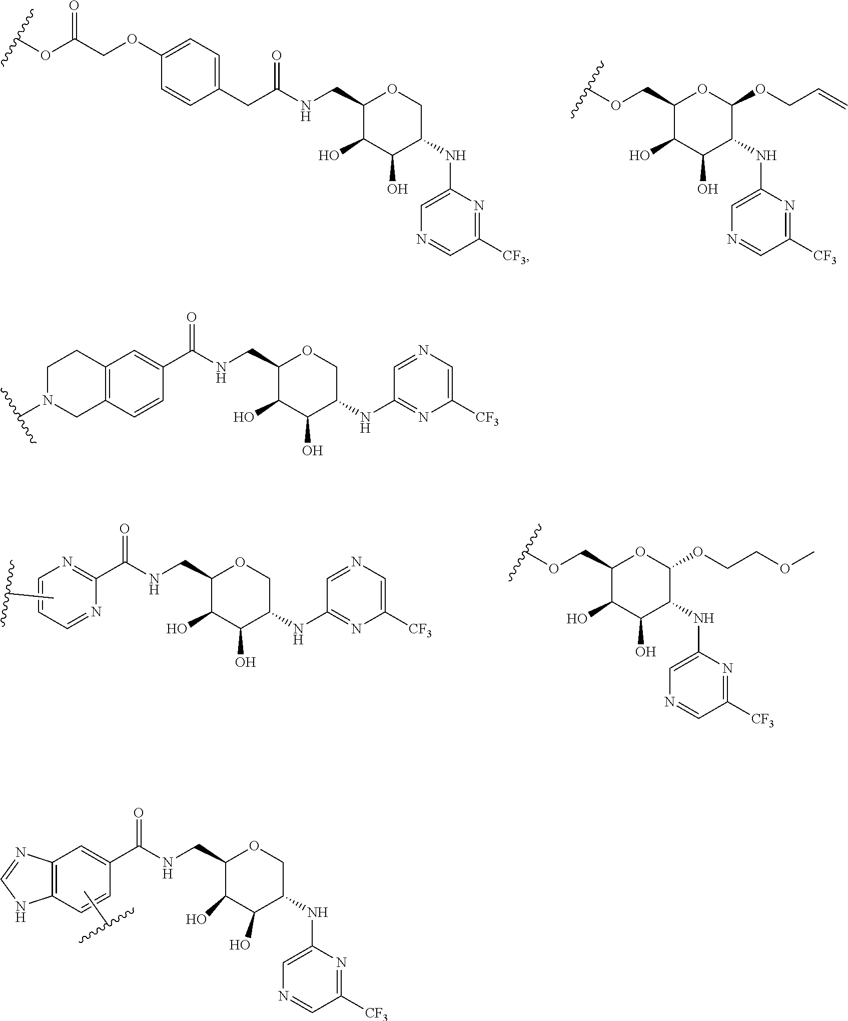

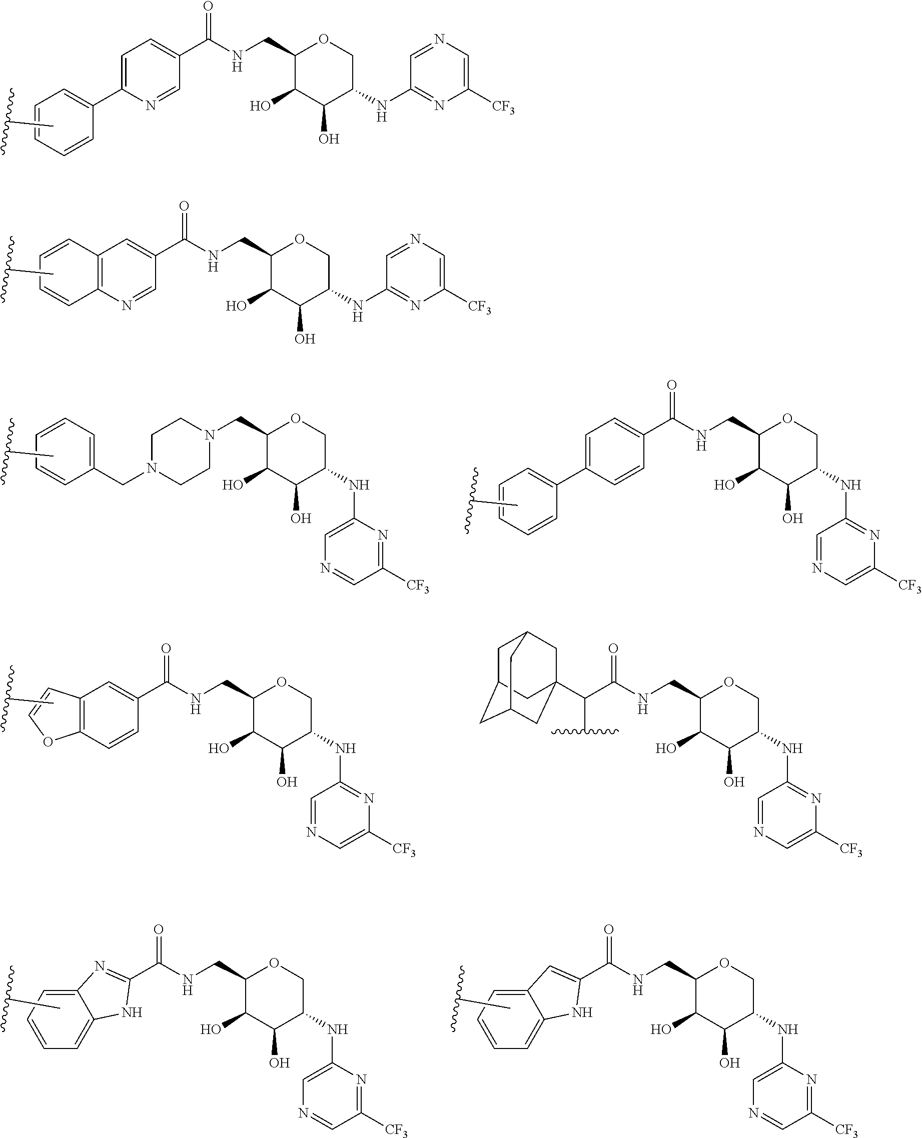

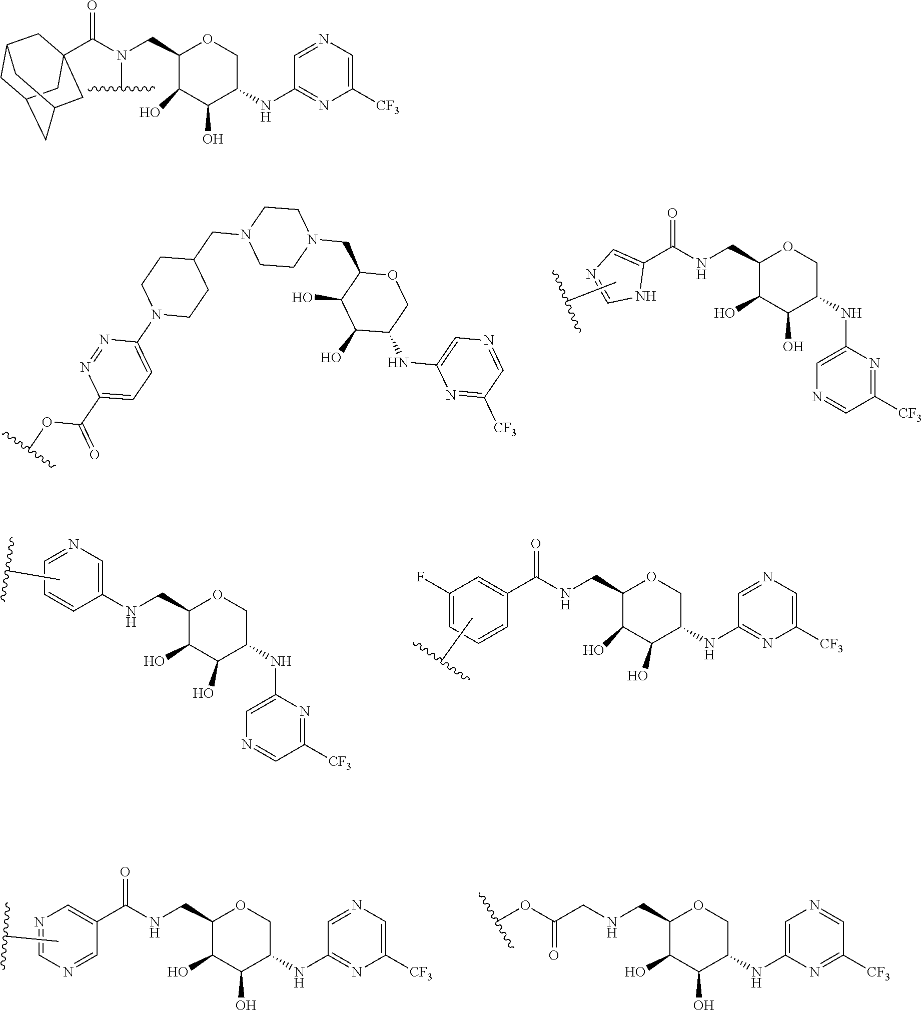

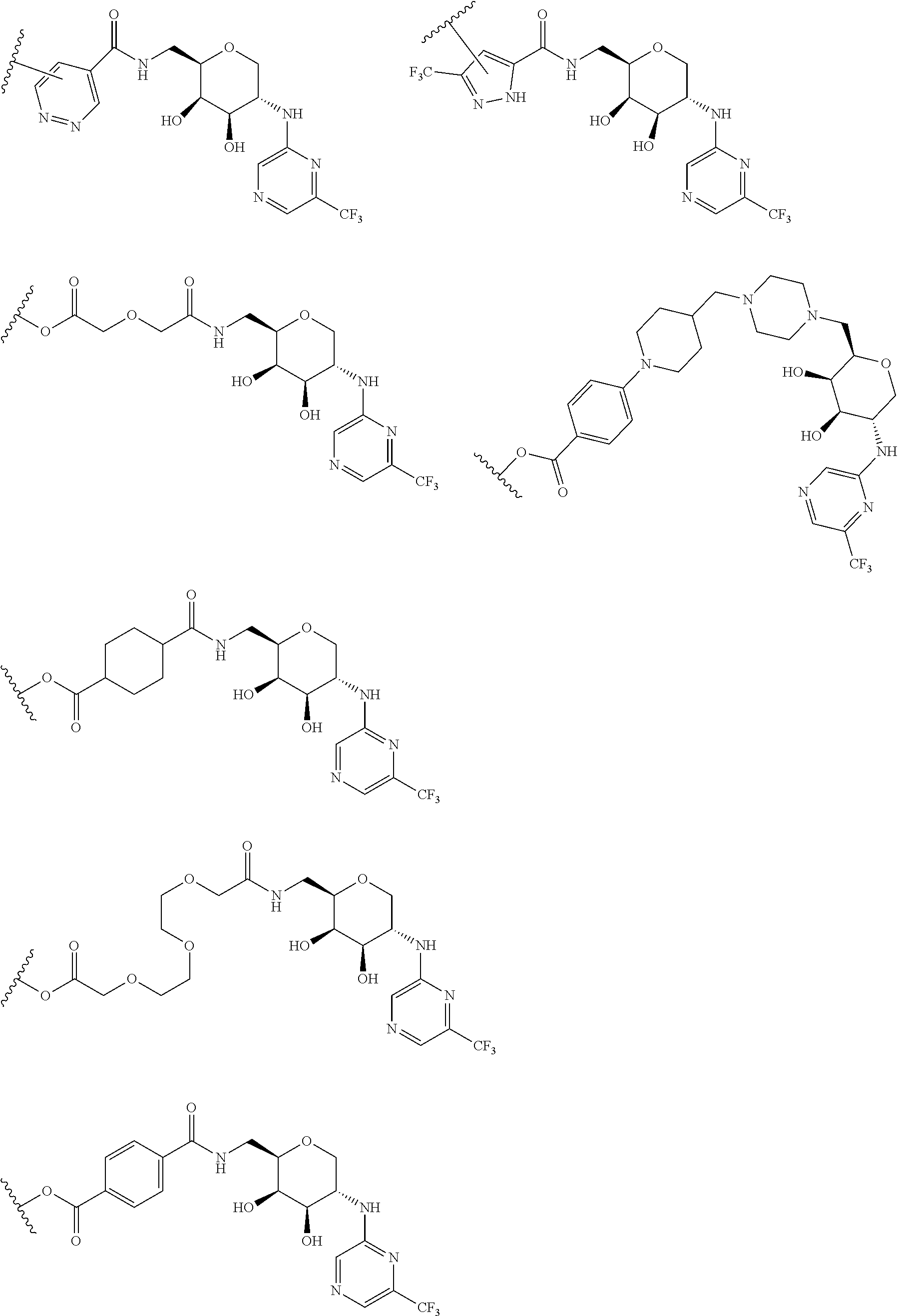

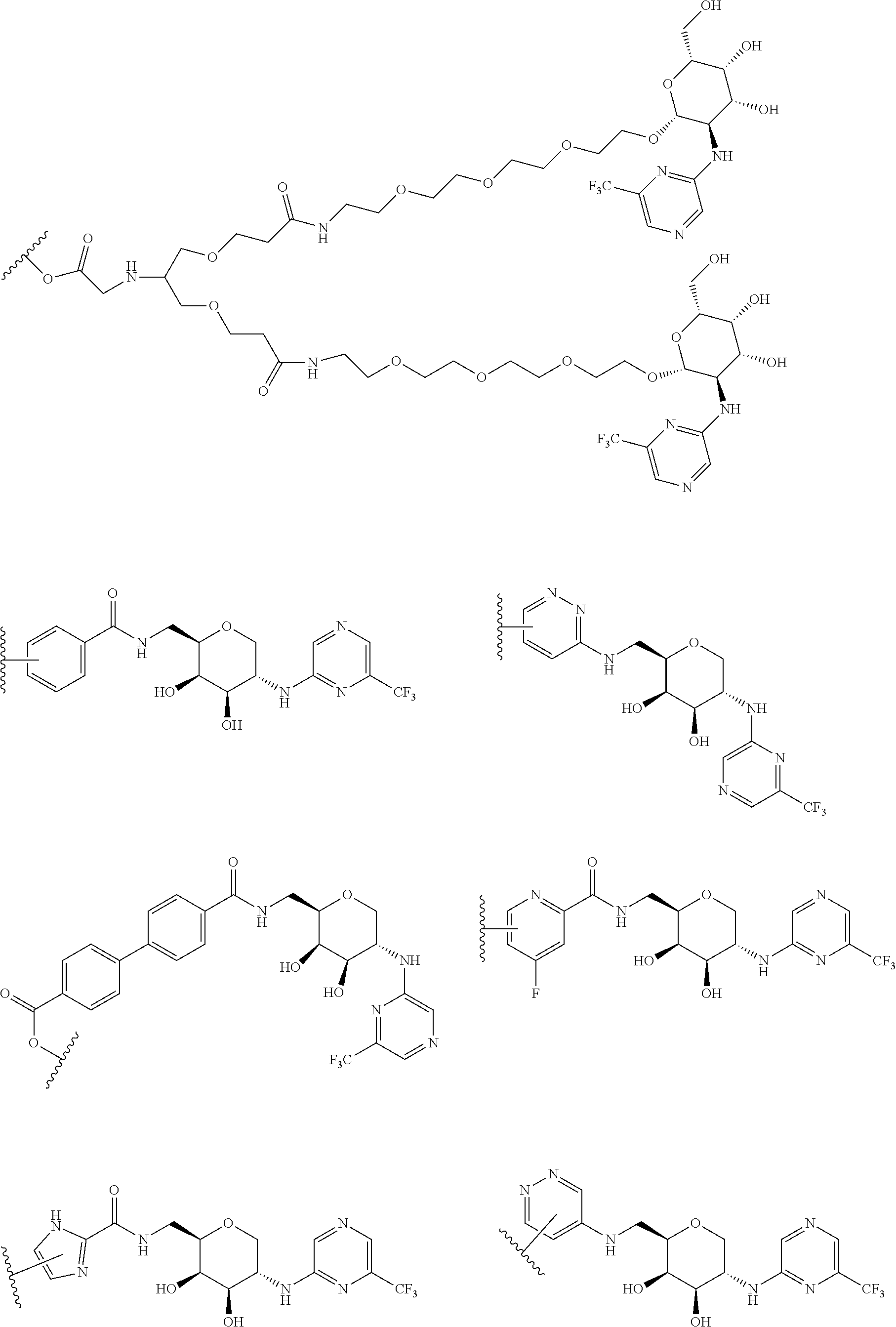

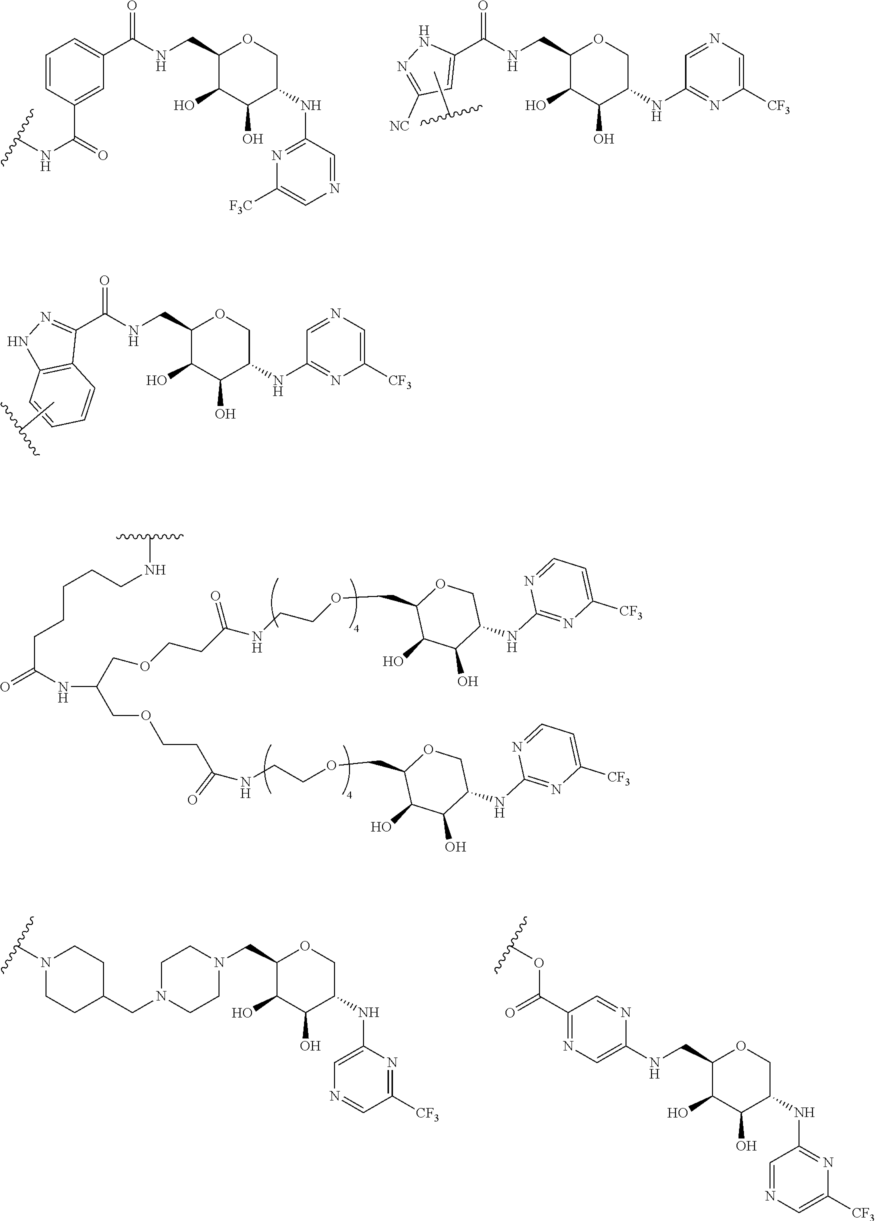

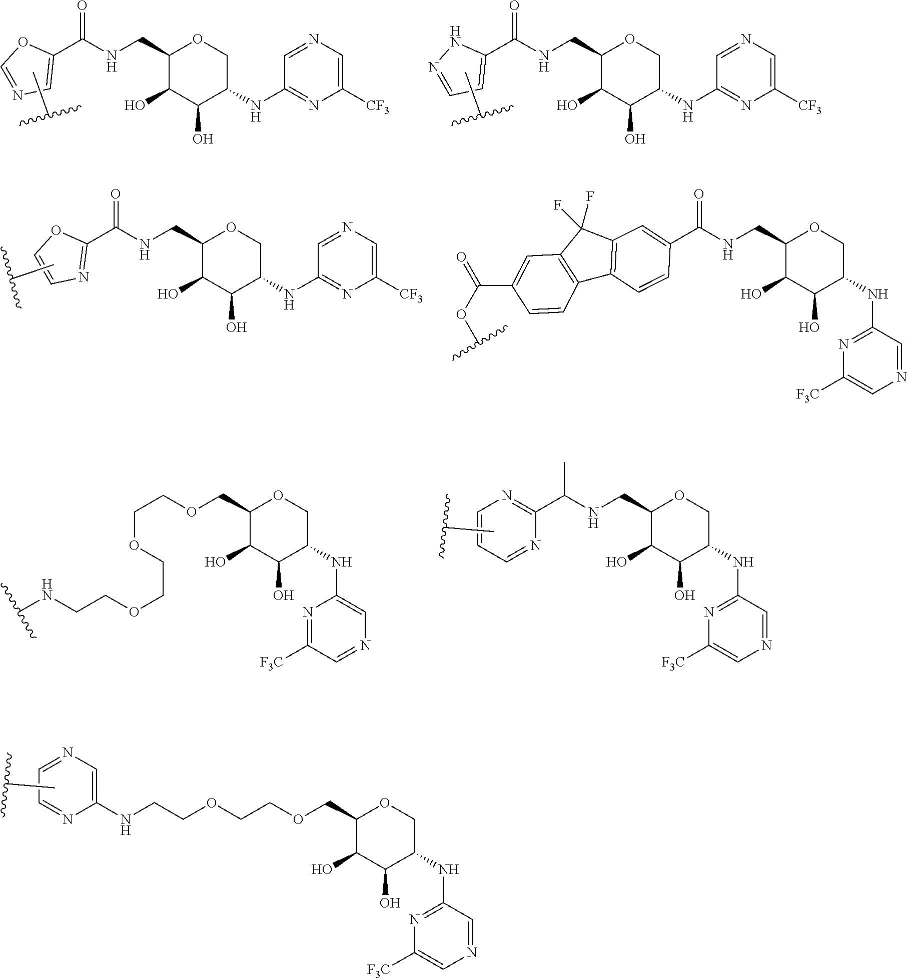

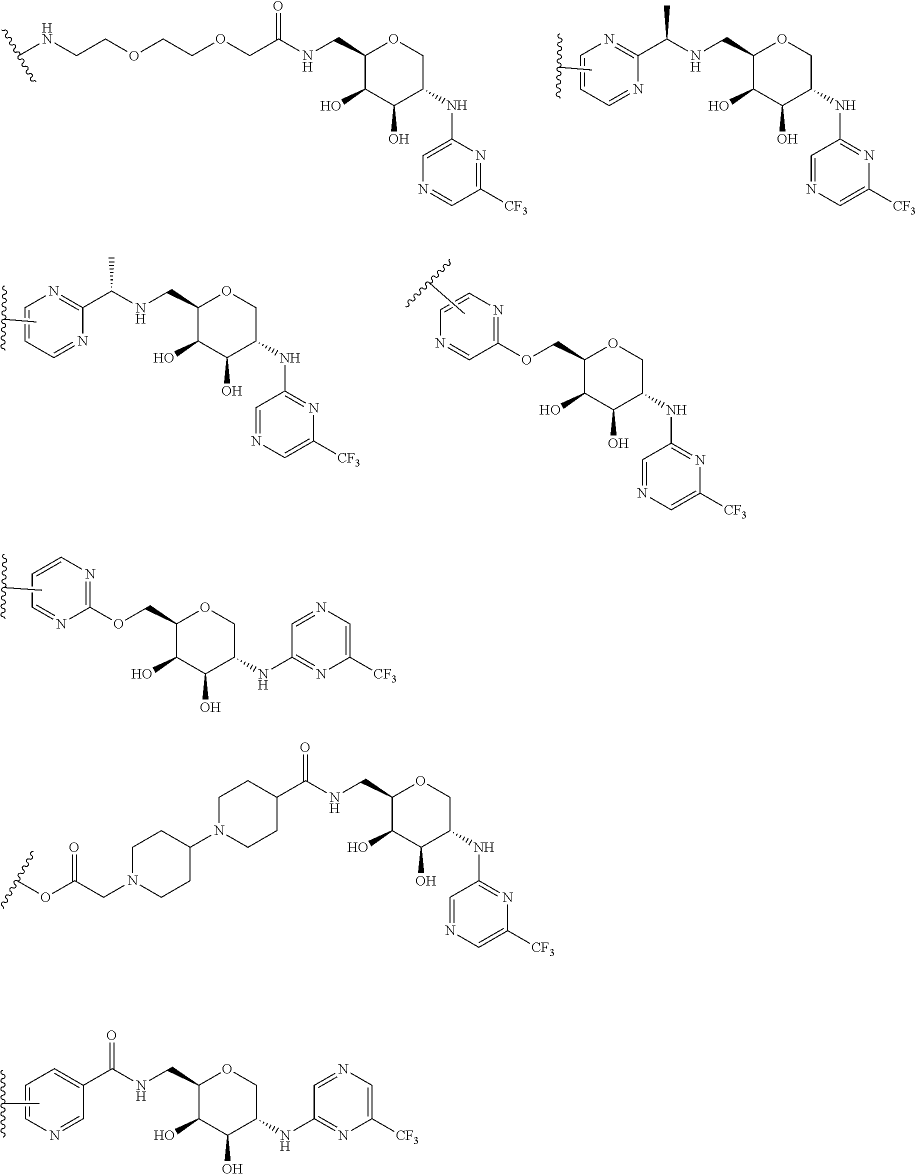

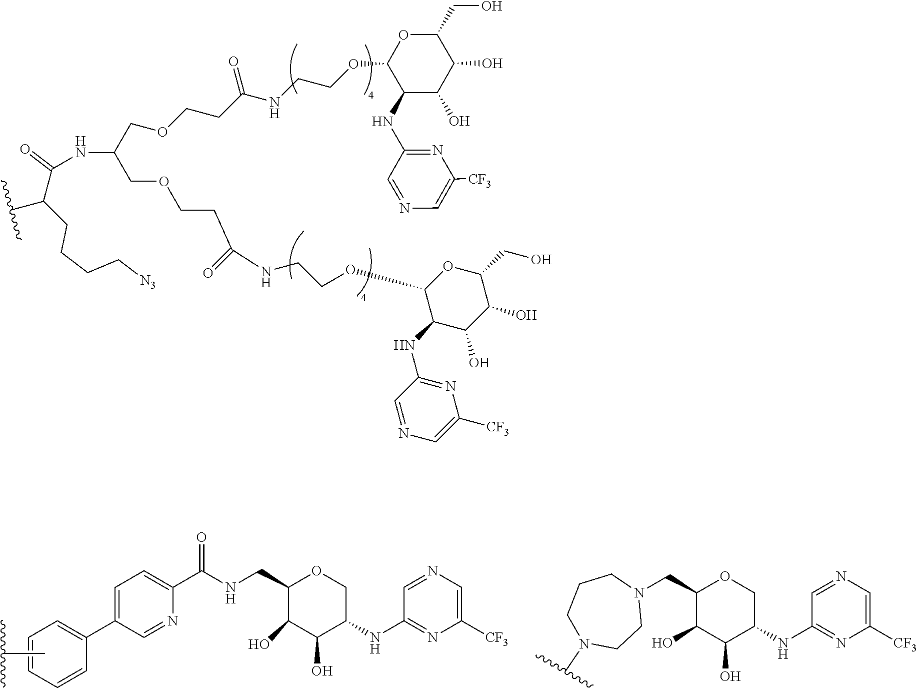

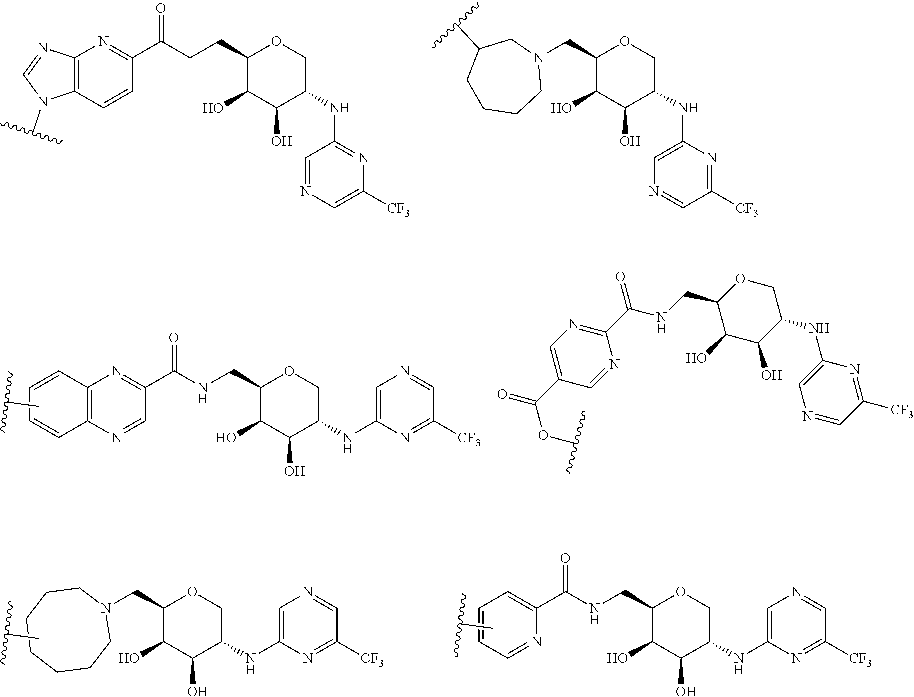

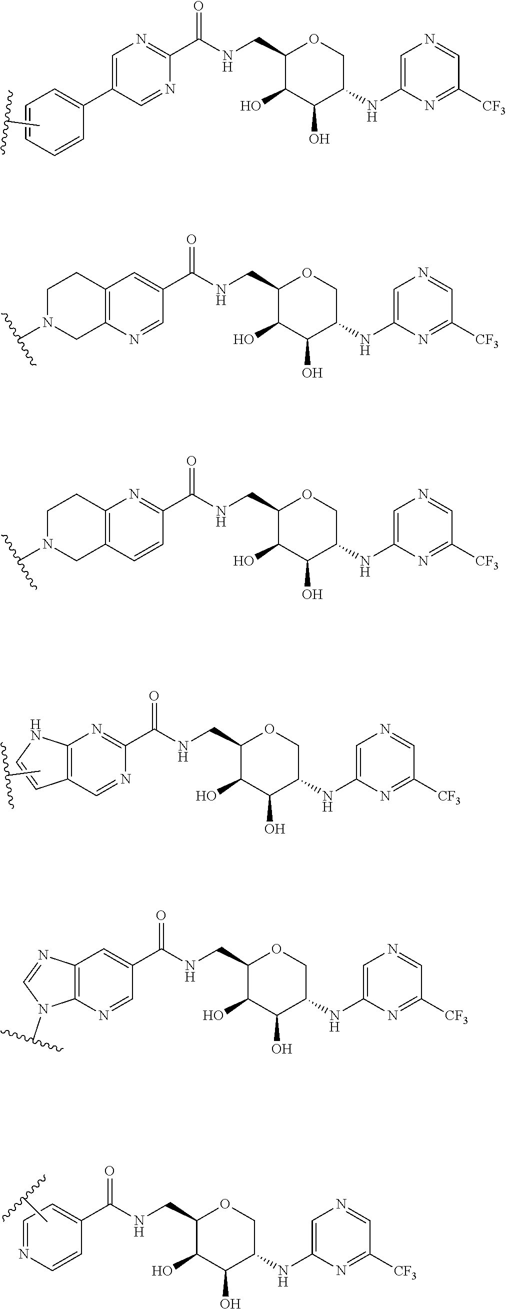

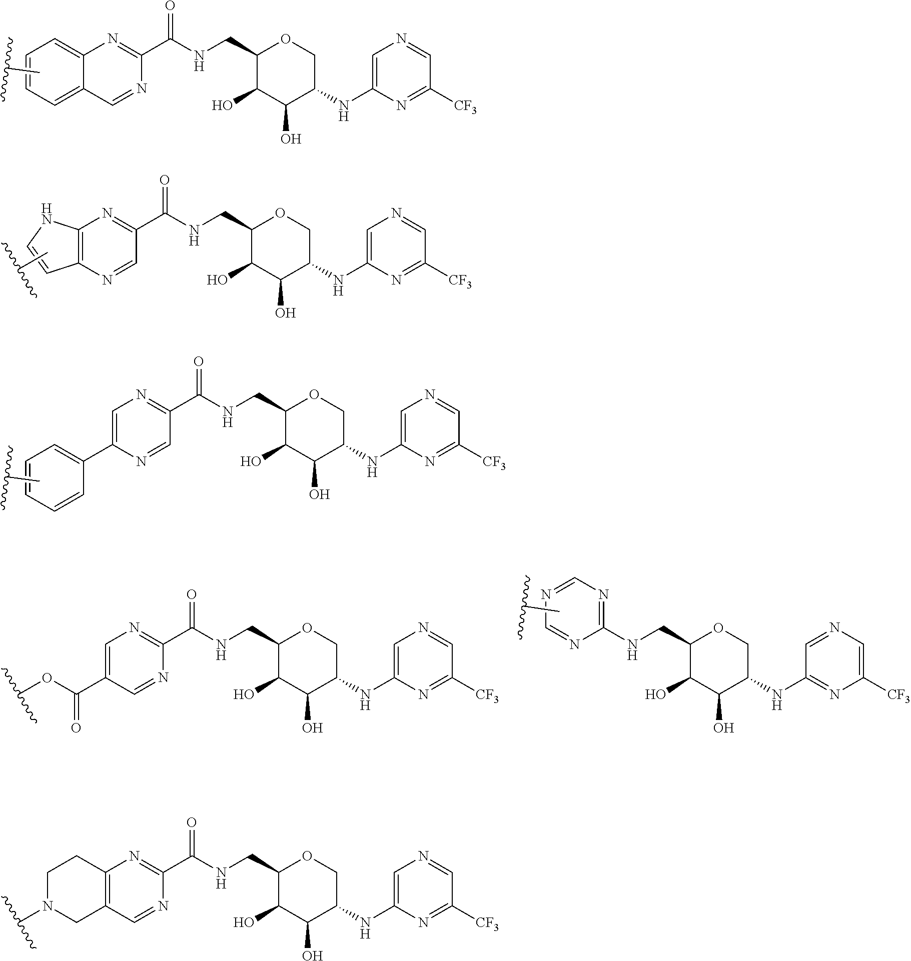

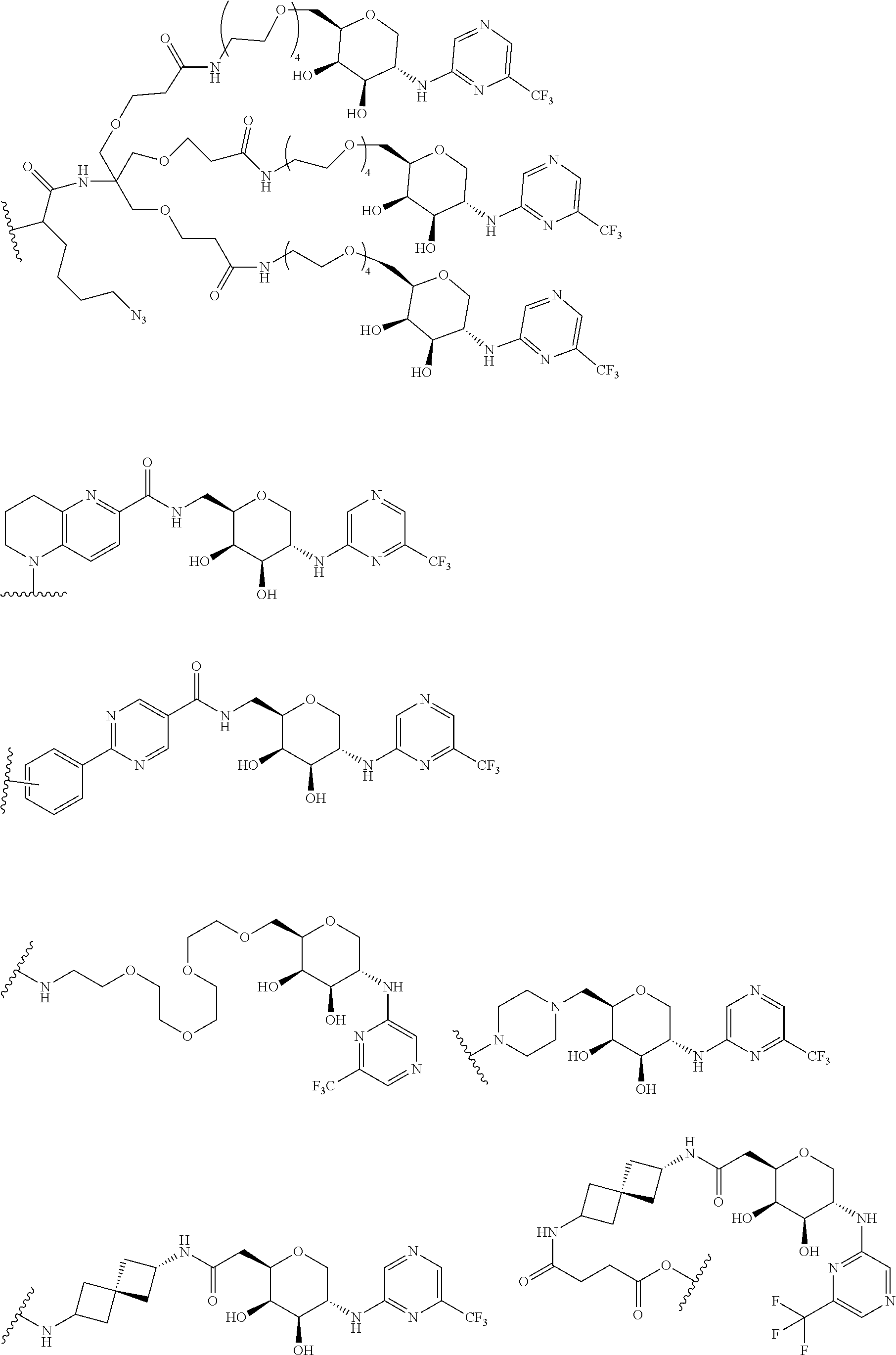

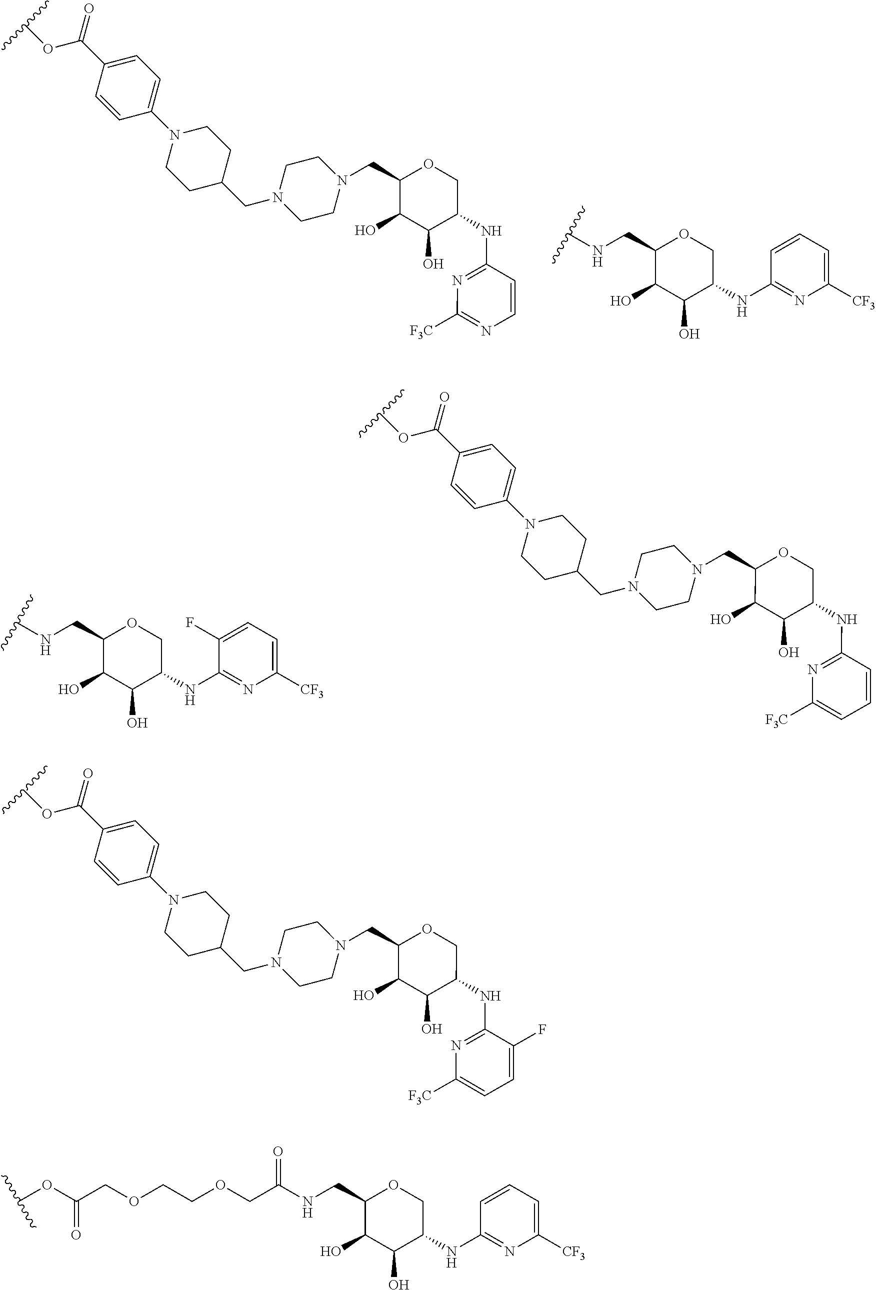 [SMILES: *OC(=O)c1ccc(N2CCC(CN3CCN(C[C@H]4OC[C@H](Nc5ccnc(C(F)(F)F)n5)[C@@H](O)[C@H]4O)CC3)CC2)cc1 ; *NC[C@H]1OC[C@H](Nc2cccc(C(F)(F)F)n2)[C@@H](O)[C@H]1O ; *NC[C@H]1OC[C@H](Nc2nc(C(F)(F)F)ccc2F)[C@@H](O)[C@H]1O ; *OC(=O)c1ccc(N2CCC(CN3CCN(C[C@H]4OC[C@H](Nc5cccc(C(F)(F)F)n5)[C@@H](O)[C@H]4O)CC3)CC2)cc1 ; *OC(=O)c1ccc(N2CCC(CN3CCN(C[C@H]4OC[C@H](Nc5nc(C(F)(F)F)ccc5F)[C@@H](O)[C@H]4O)CC3)CC2)cc1 ; *OC(=O)COCCOCC(=O)NC[C@H]1OC[C@H](Nc2cccc(C(F)(F)F)n2)[C@@H](O)[C@H]1O]

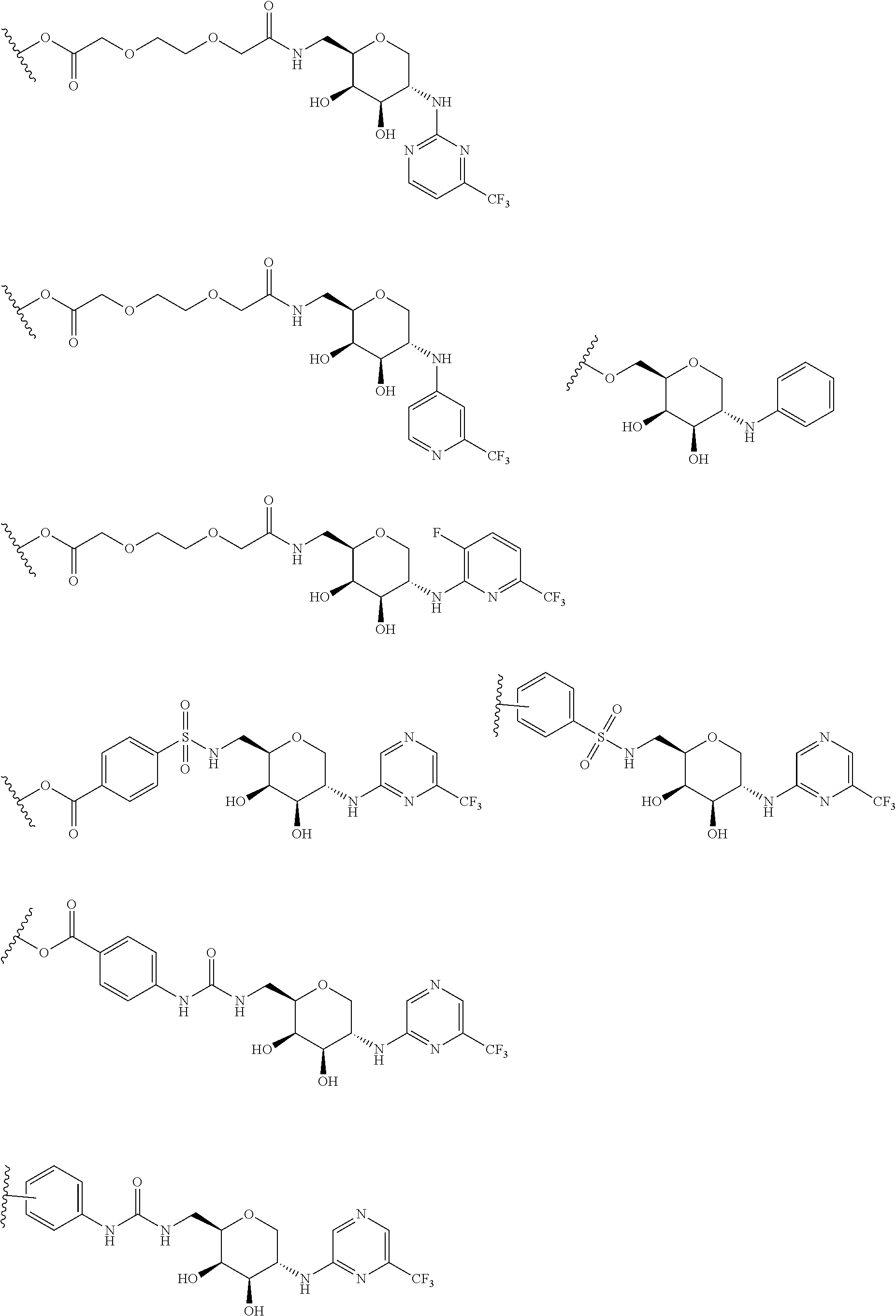

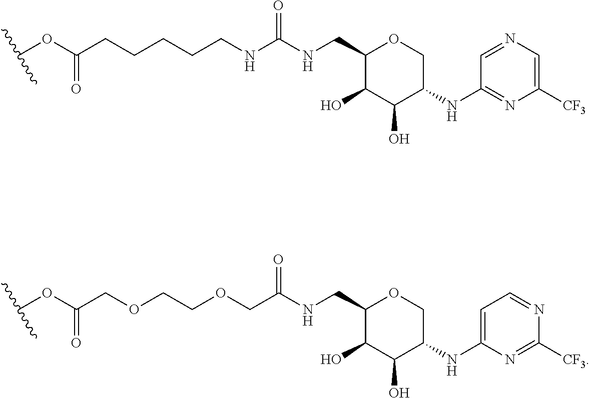 [SMILES: *OC(=O)CCCCCNC(=O)NC[C@H]1OC[C@H](Nc2cncc(C(F)(F)F)n2)[C@@H](O)[C@H]1O ; *OC(=O)COCCOCC(=O)NC[C@H]1OC[C@H](Nc2ccnc(C(F)(F)F)n2)[C@@H](O)[C@H]1O]

In certain embodiments the ASGPR Binding Ligand is selected from:

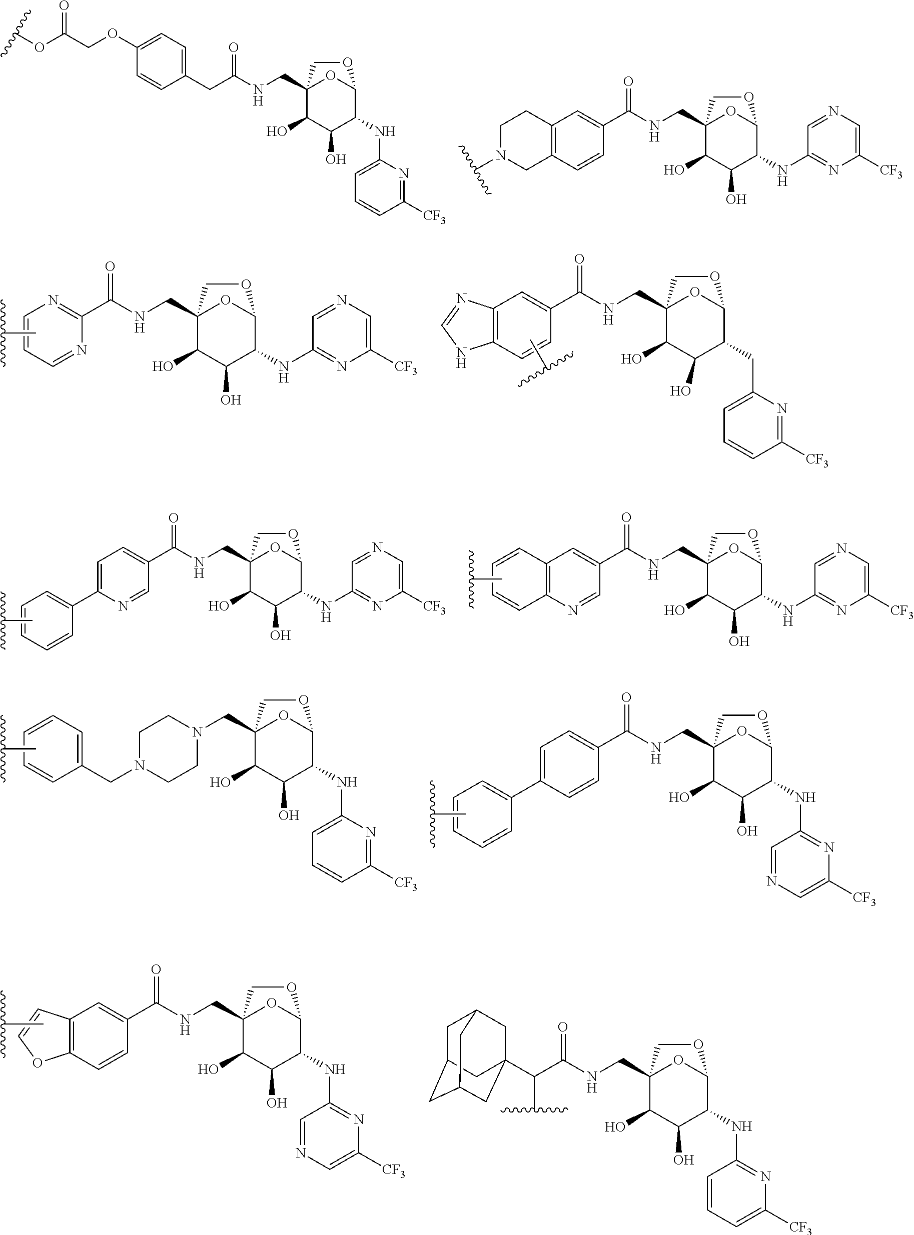

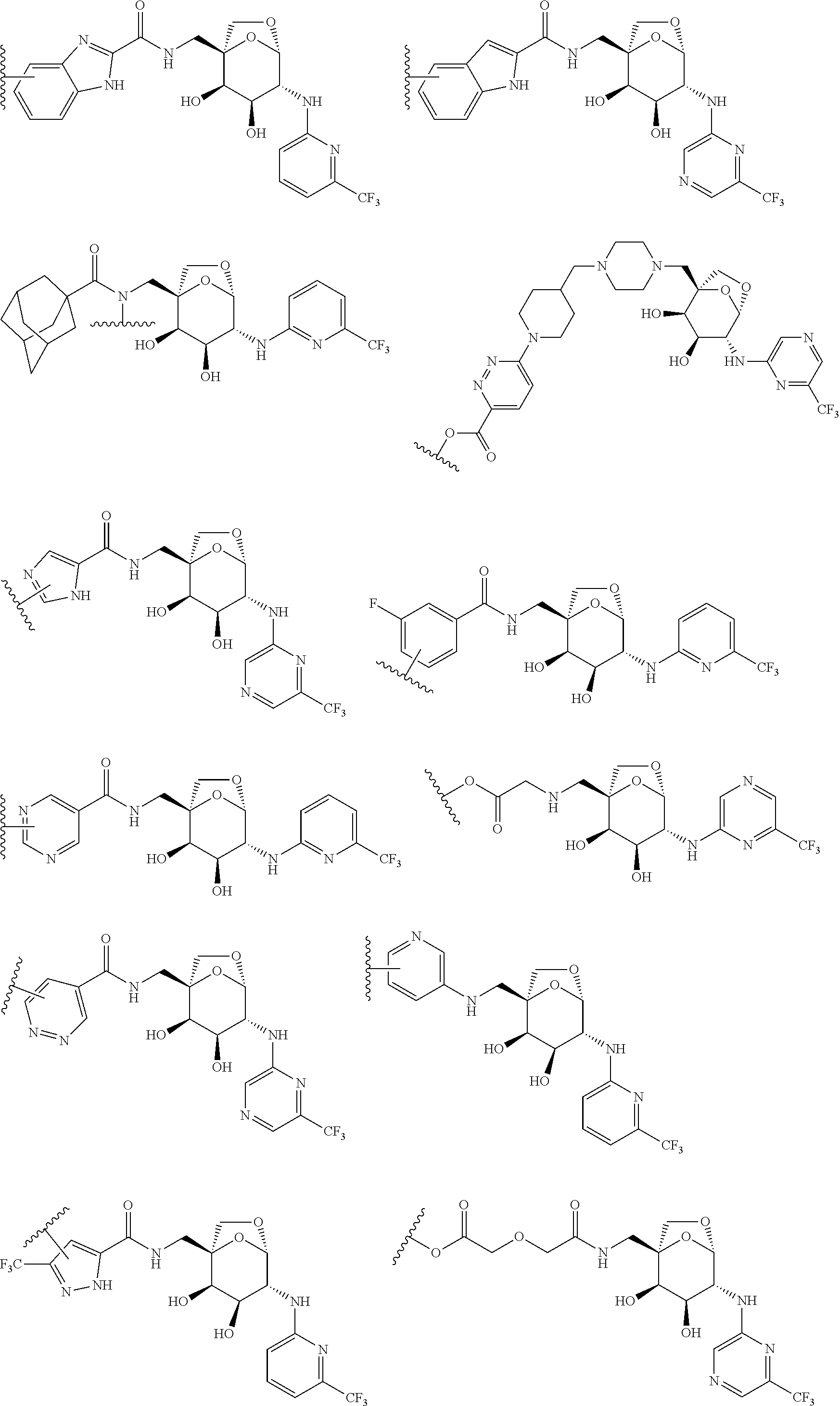

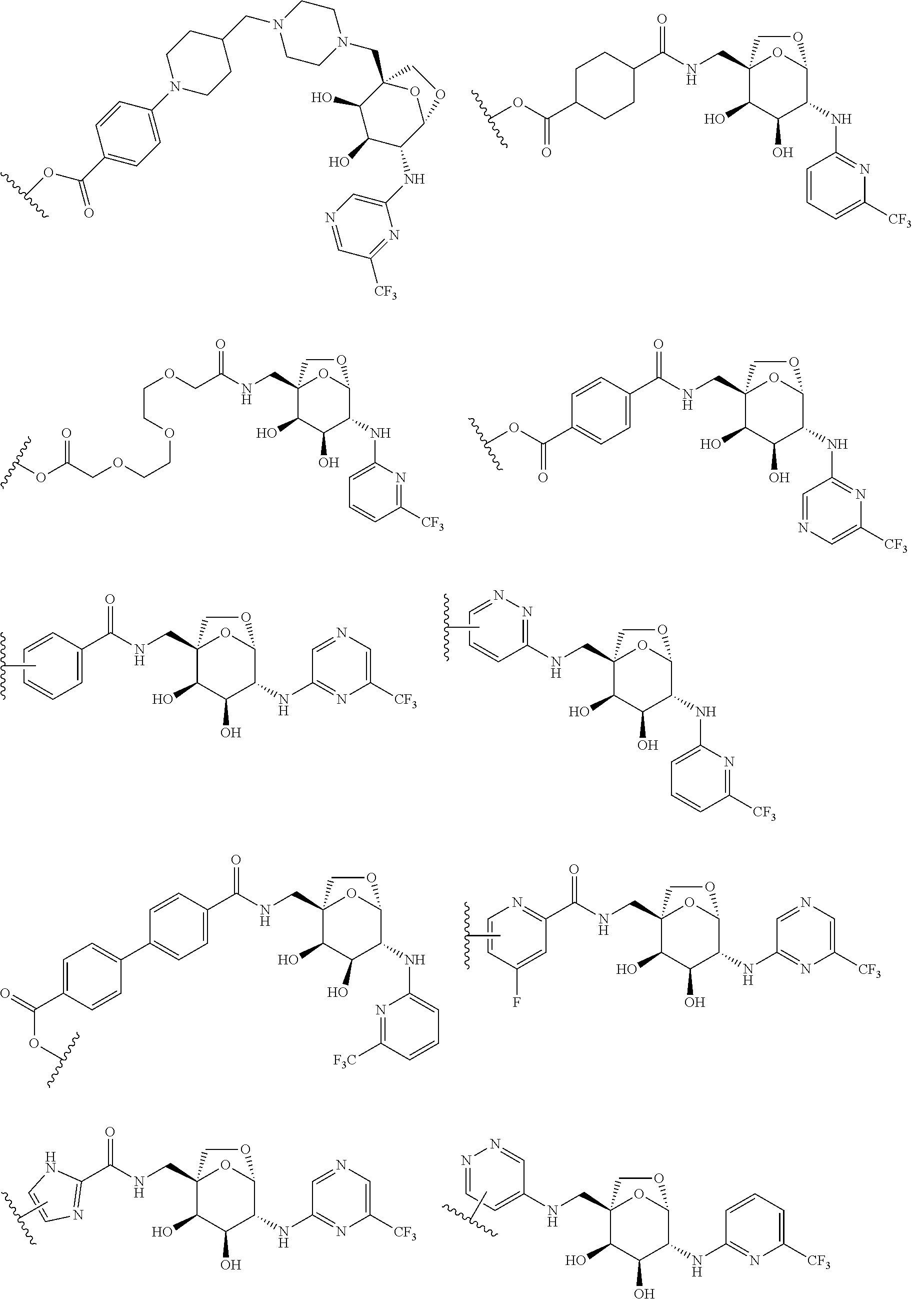

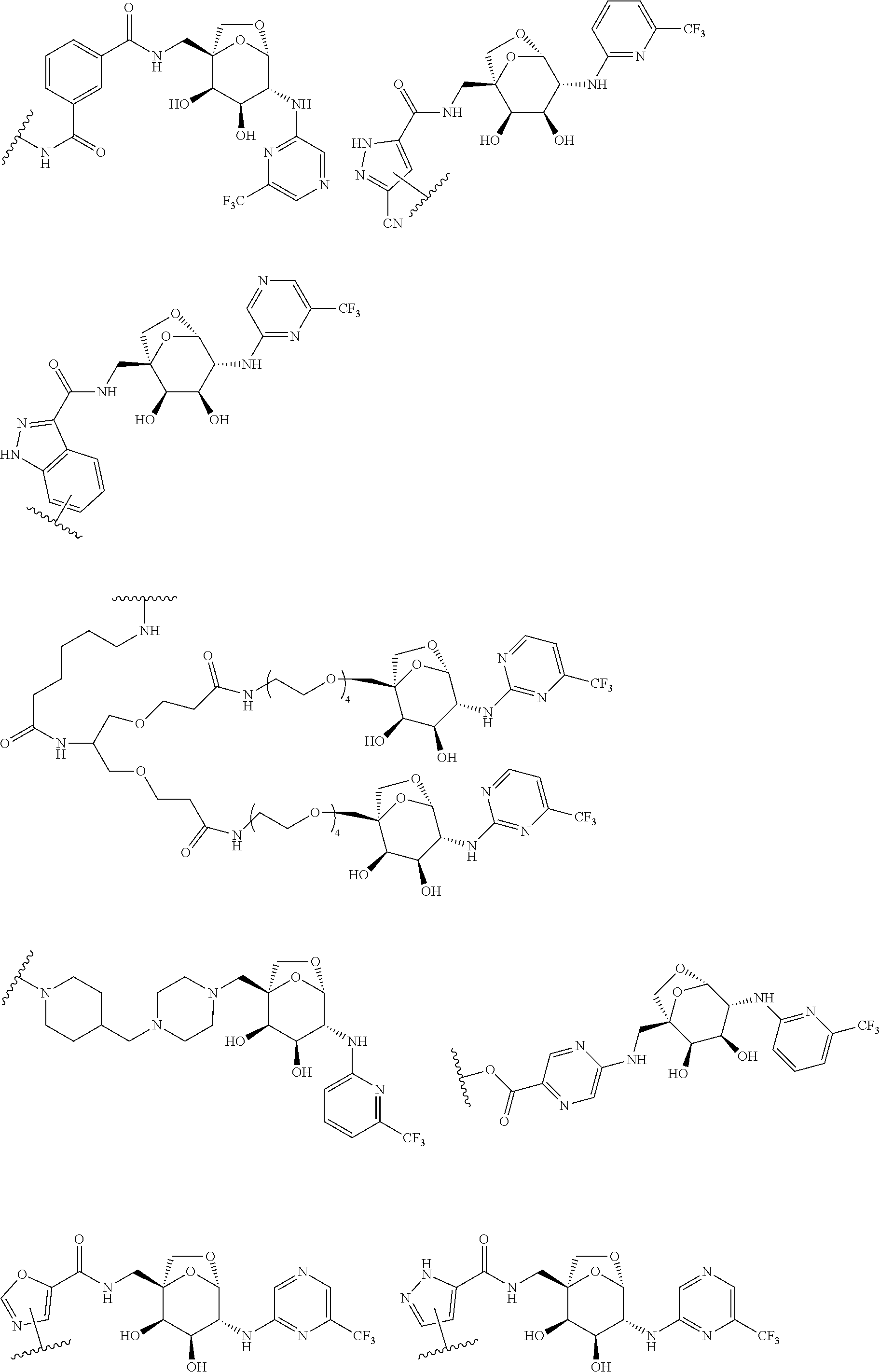

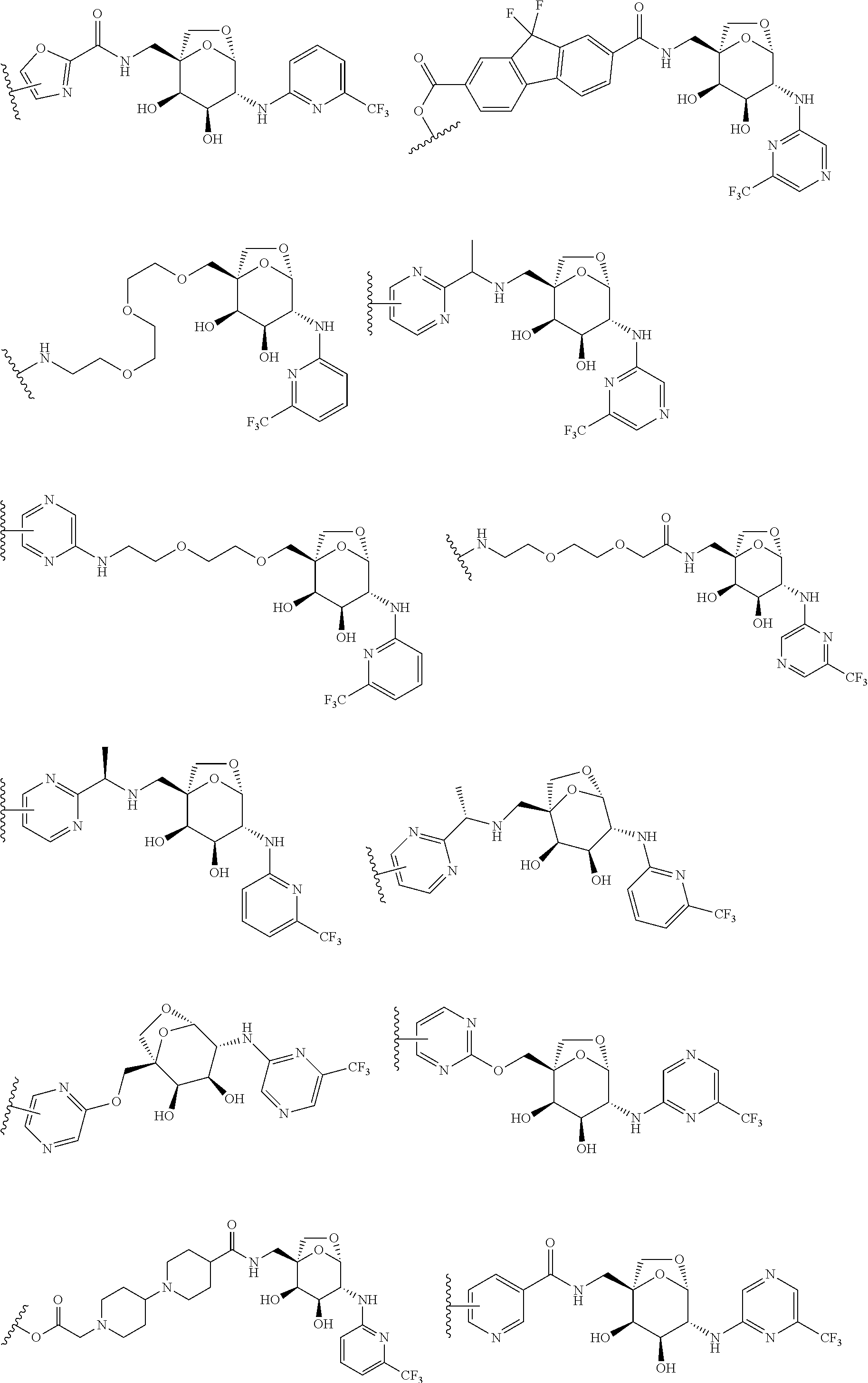

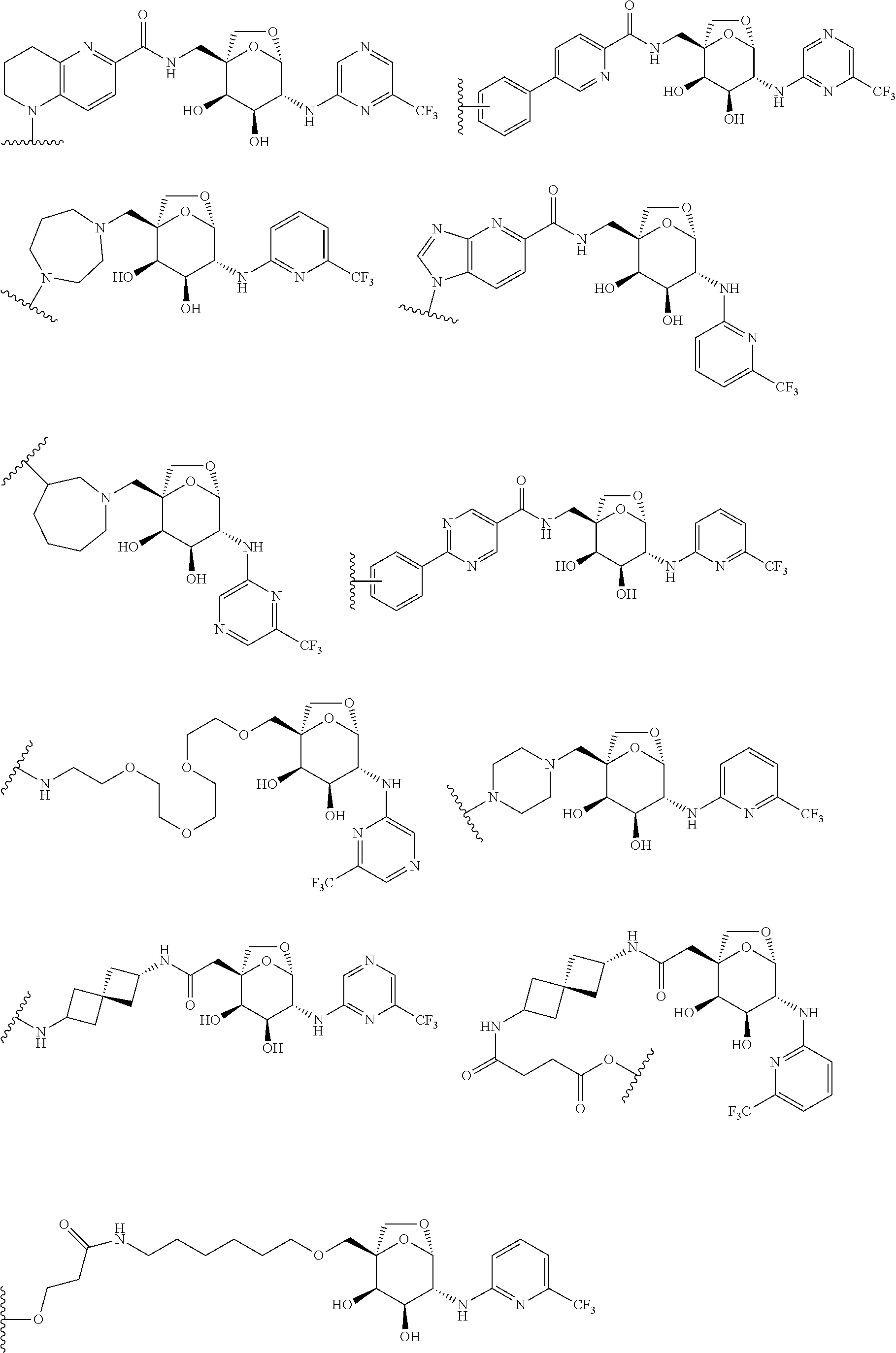

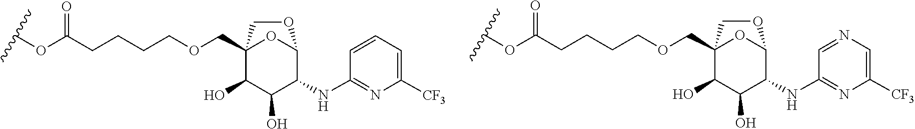 [SMILES: *OC(=O)CCCCOC[C@@]12CO[C@@H](O1)[C@H](Nc1cccc(C(F)(F)F)n1)[C@@H](O)[C@H]2O ; *OC(=O)CCCCOC[C@@]12CO[C@@H](O1)[C@H](Nc1cncc(C(F)(F)F)n1)[C@@H](O)[C@H]2O]

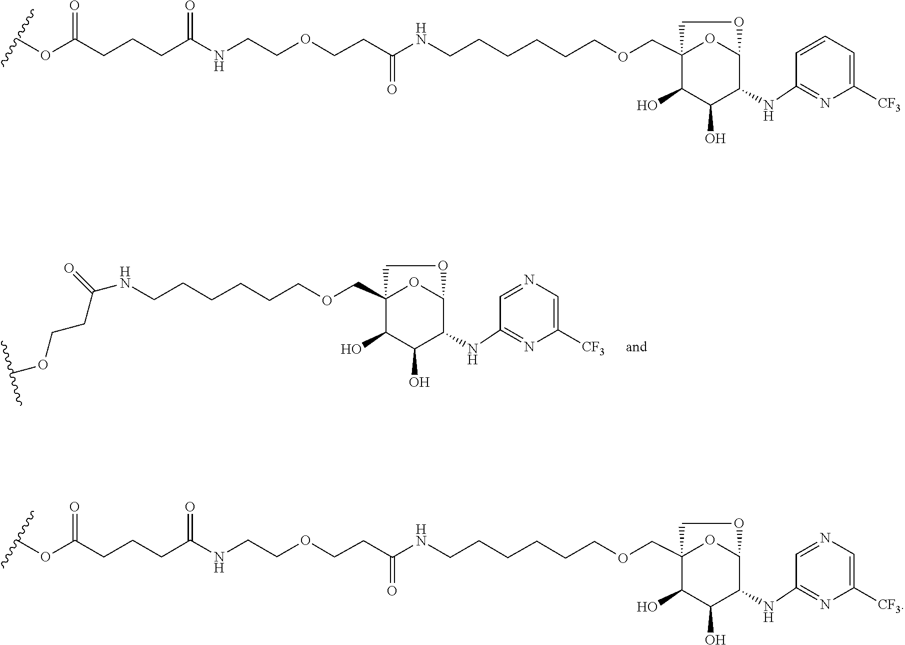 [SMILES: *OC(=O)CCCC(=O)NCCOCCC(=O)NCCCCCCOC[C@@]12CO[C@@H](O1)[C@H](Nc1cccc(C(F)(F)F)n1)[C@@H](O)[C@H]2O ; *OCCC(=O)NCCCCCCOC[C@@]12CO[C@@H](O1)[C@H](Nc1cncc(C(F)(F)F)n1)[C@@H](O)[C@H]2O ; *OC(=O)CCCC(=O)NCCOCCC(=O)NCCCCCCOC[C@@]12CO[C@@H](O1)[C@H](Nc1cncc(C(F)(F)F)n1)[C@@H](O)[C@H]2O]

In certain embodiments the compound of the present invention is:

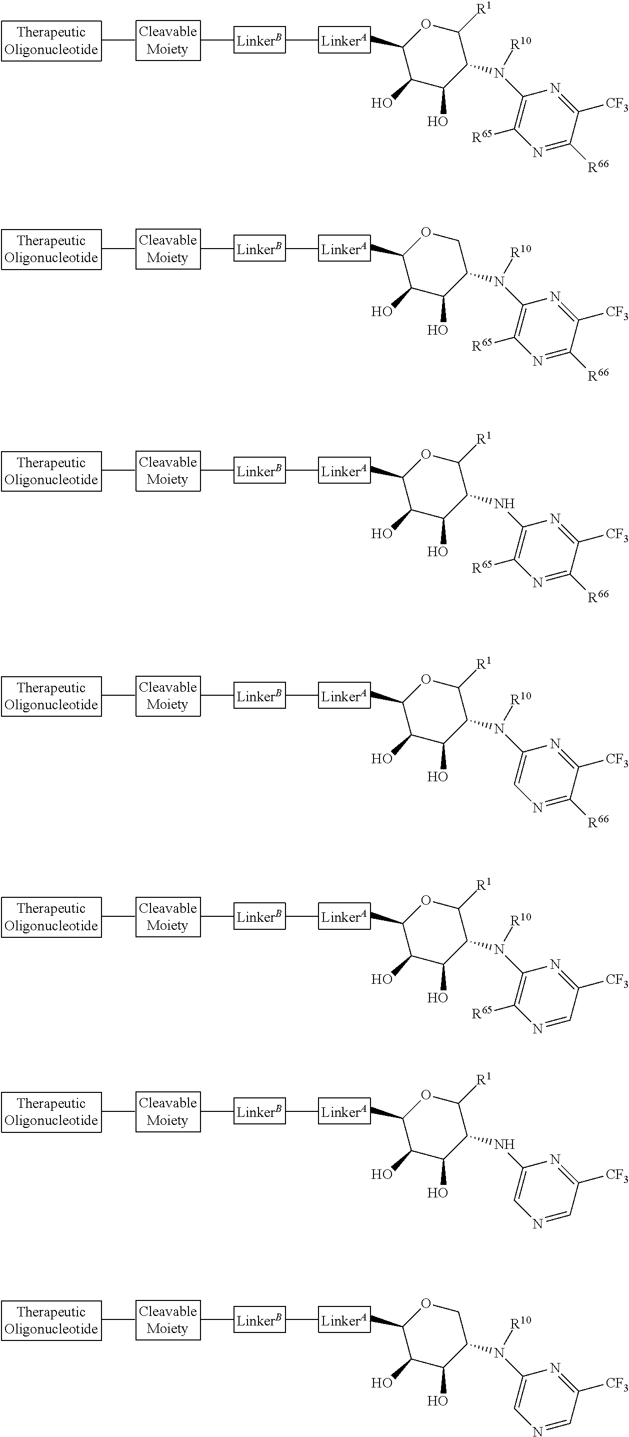

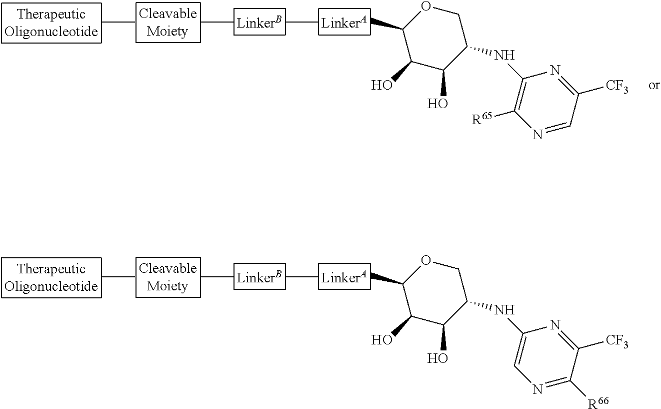

or a pharmaceutically acceptable salt thereof.

In certain embodiments the compound of the present invention is:

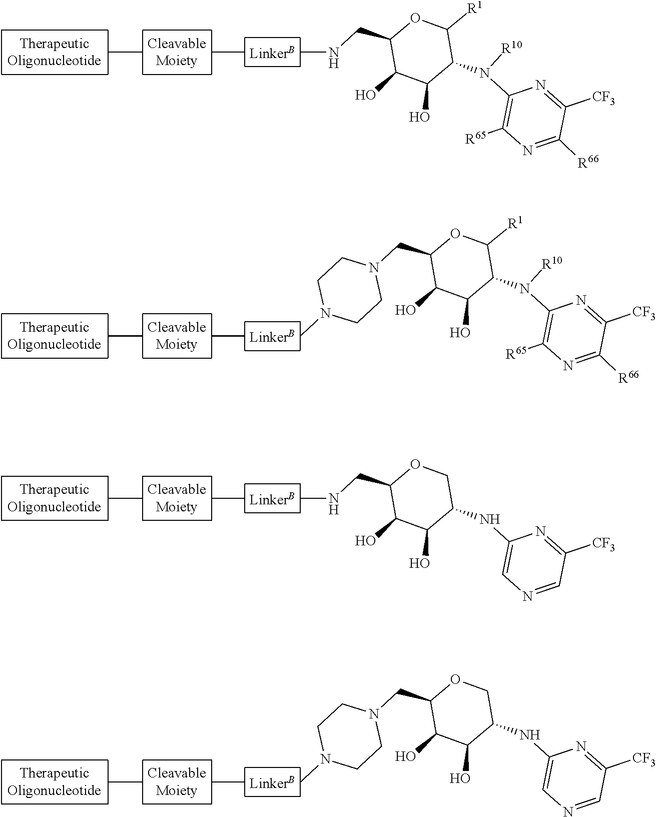

or a pharmaceutically acceptable salt thereof.

In certain embodiments the compound of the present invention is

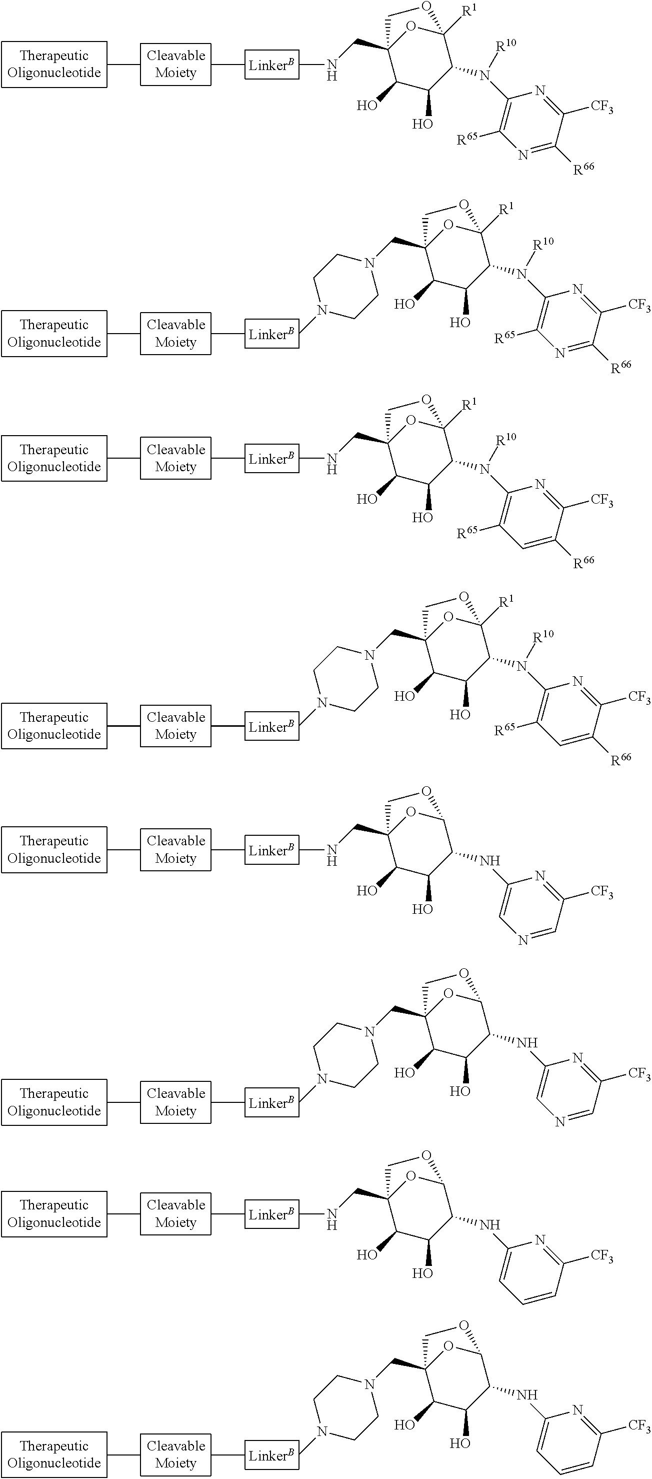

or a pharmaceutically acceptable salt thereof.

In certain embodiments the compound of the present invention is:

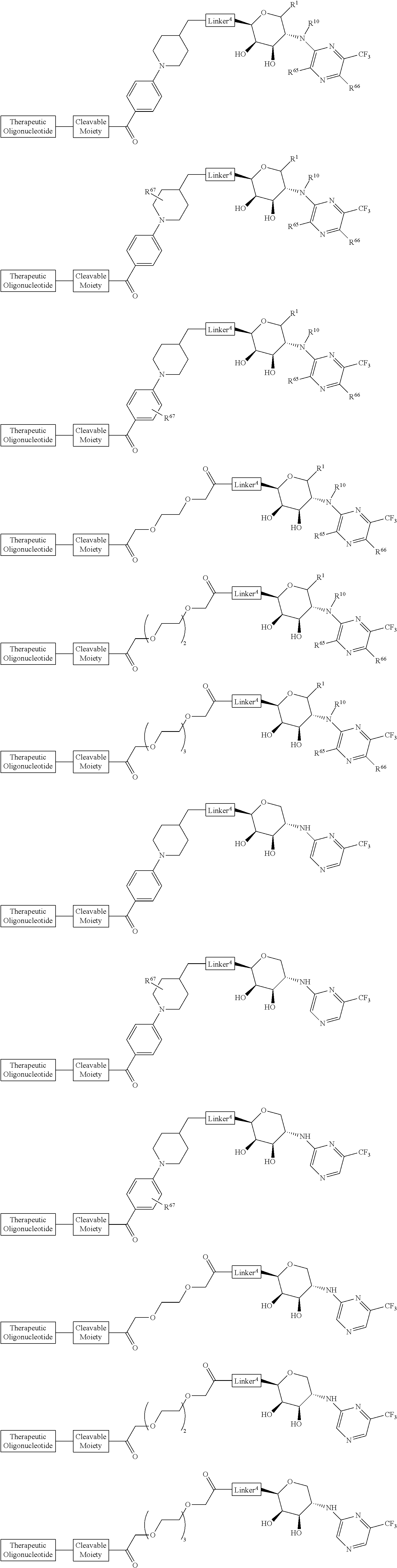

or a pharmaceutically acceptable salt thereof.

In certain embodiments the compound of the present invention is:

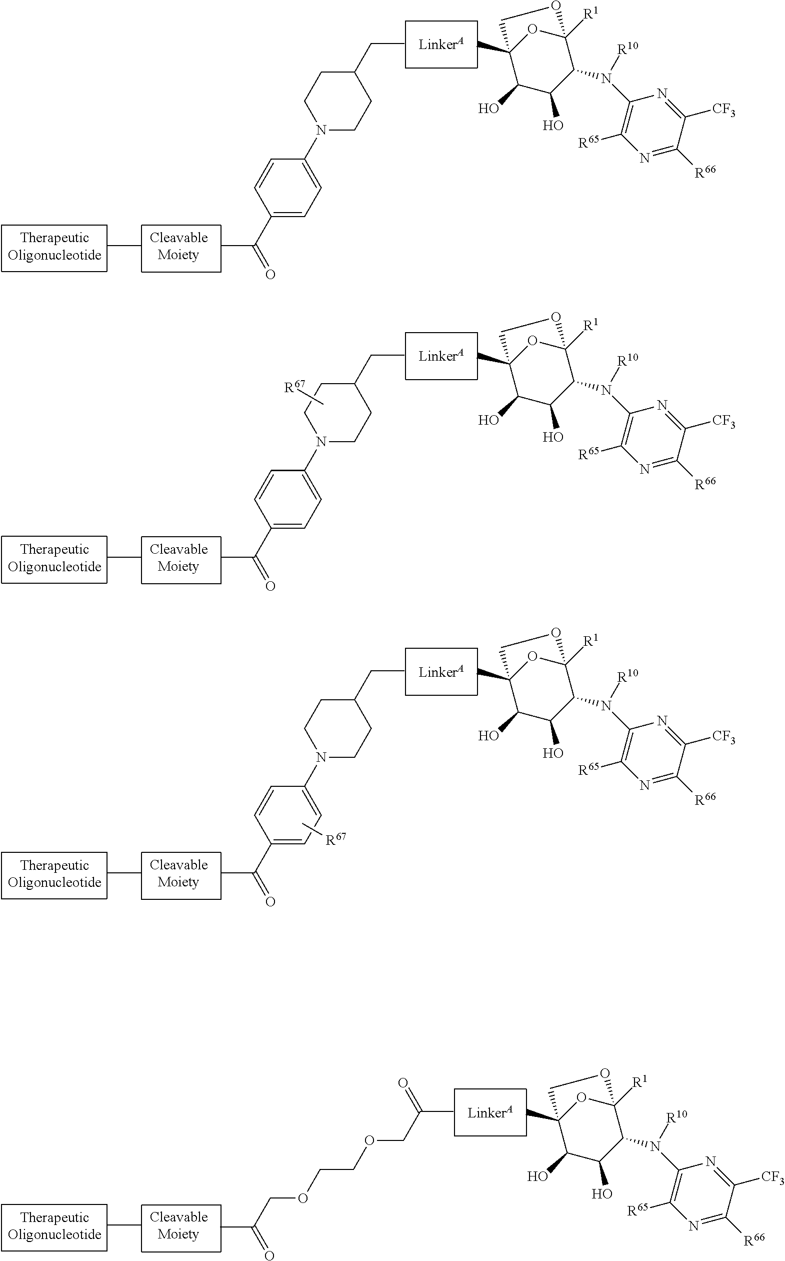

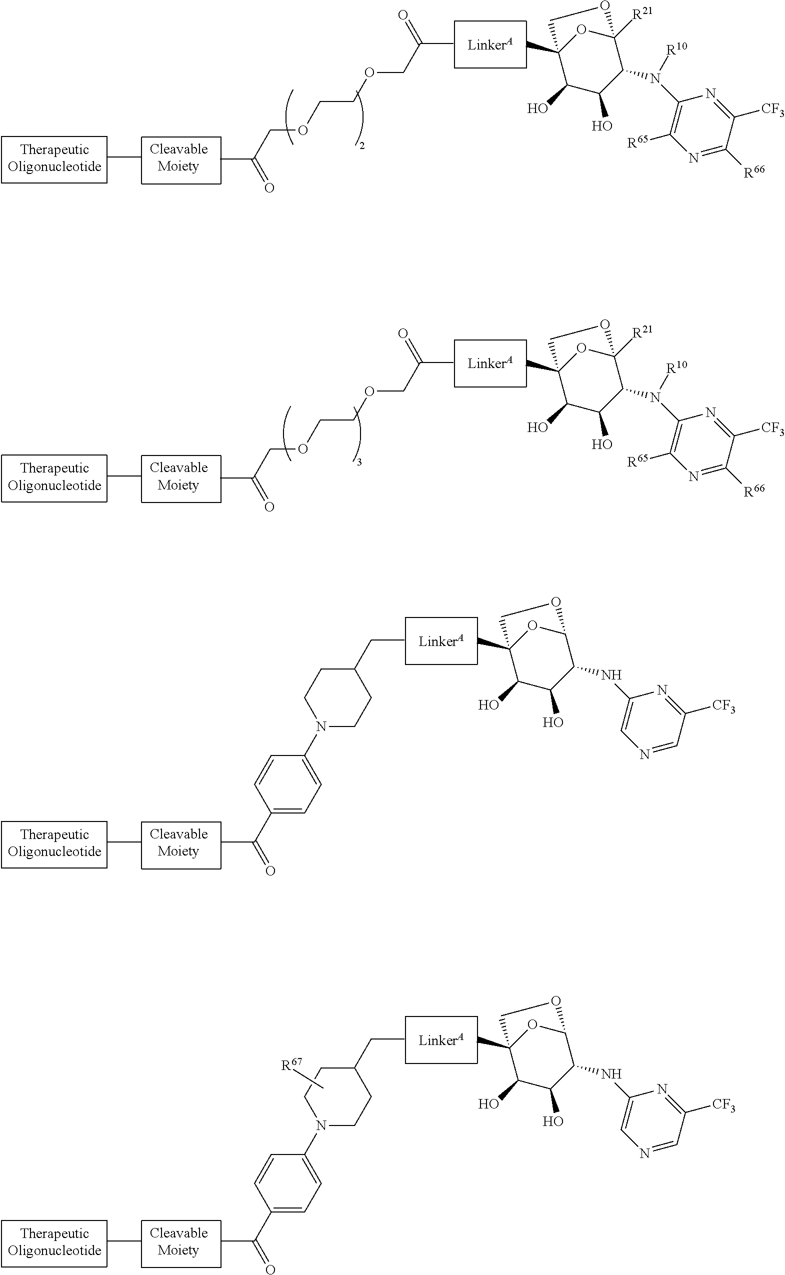

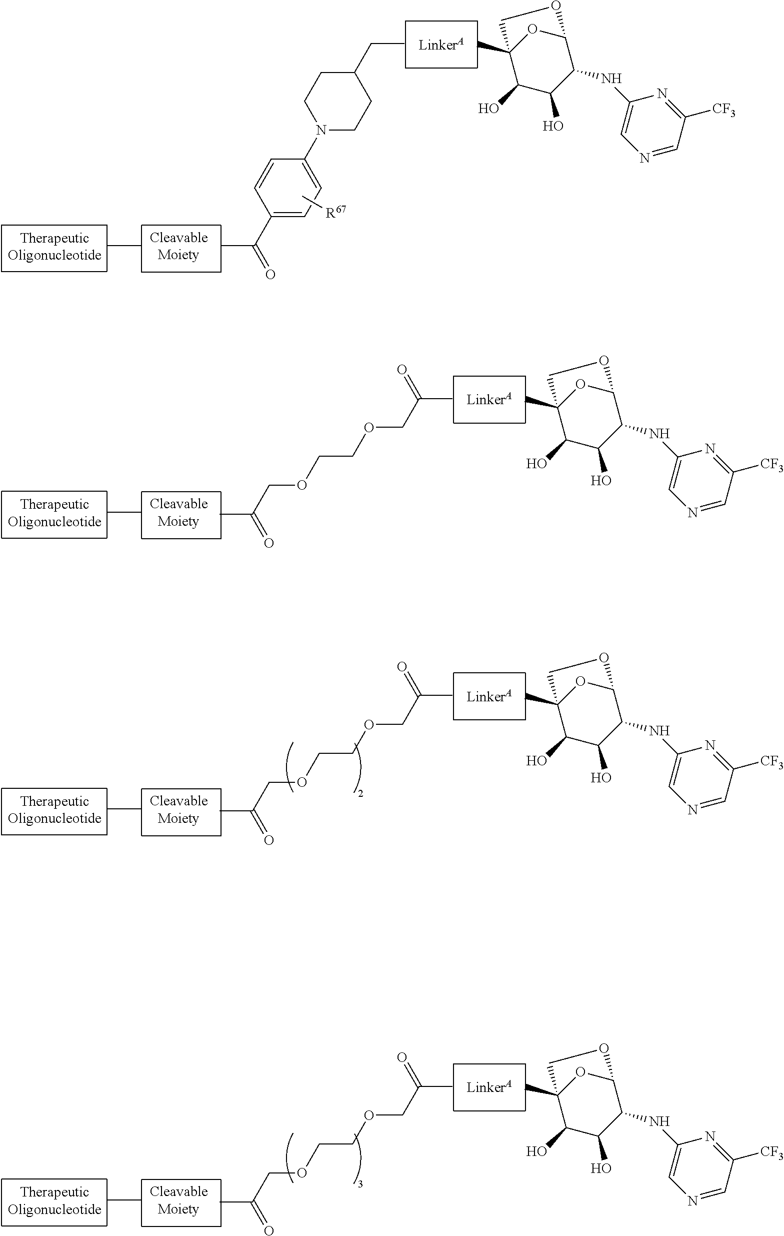

or a pharmaceutically acceptable salt thereof.

In certain embodiments the compound of the present invention is:

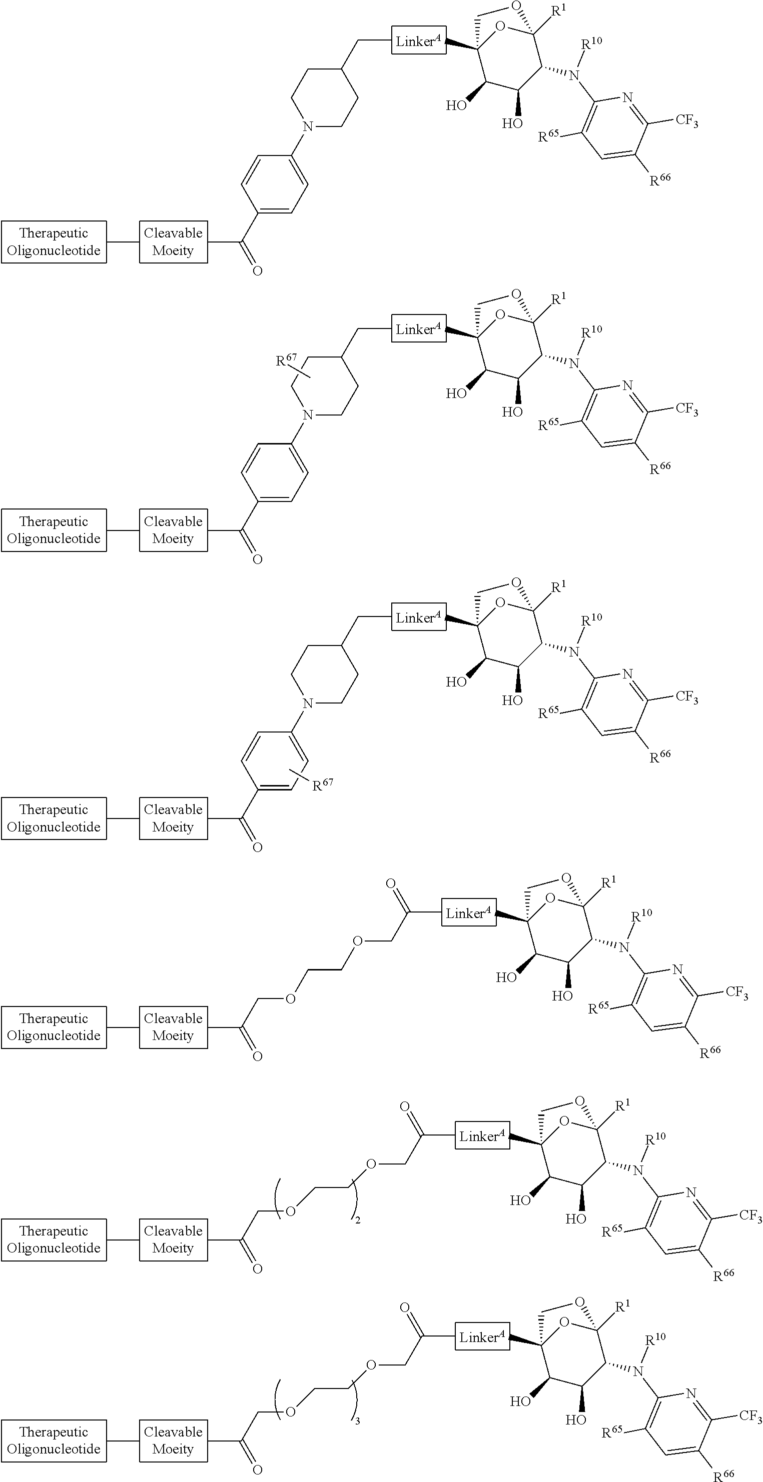

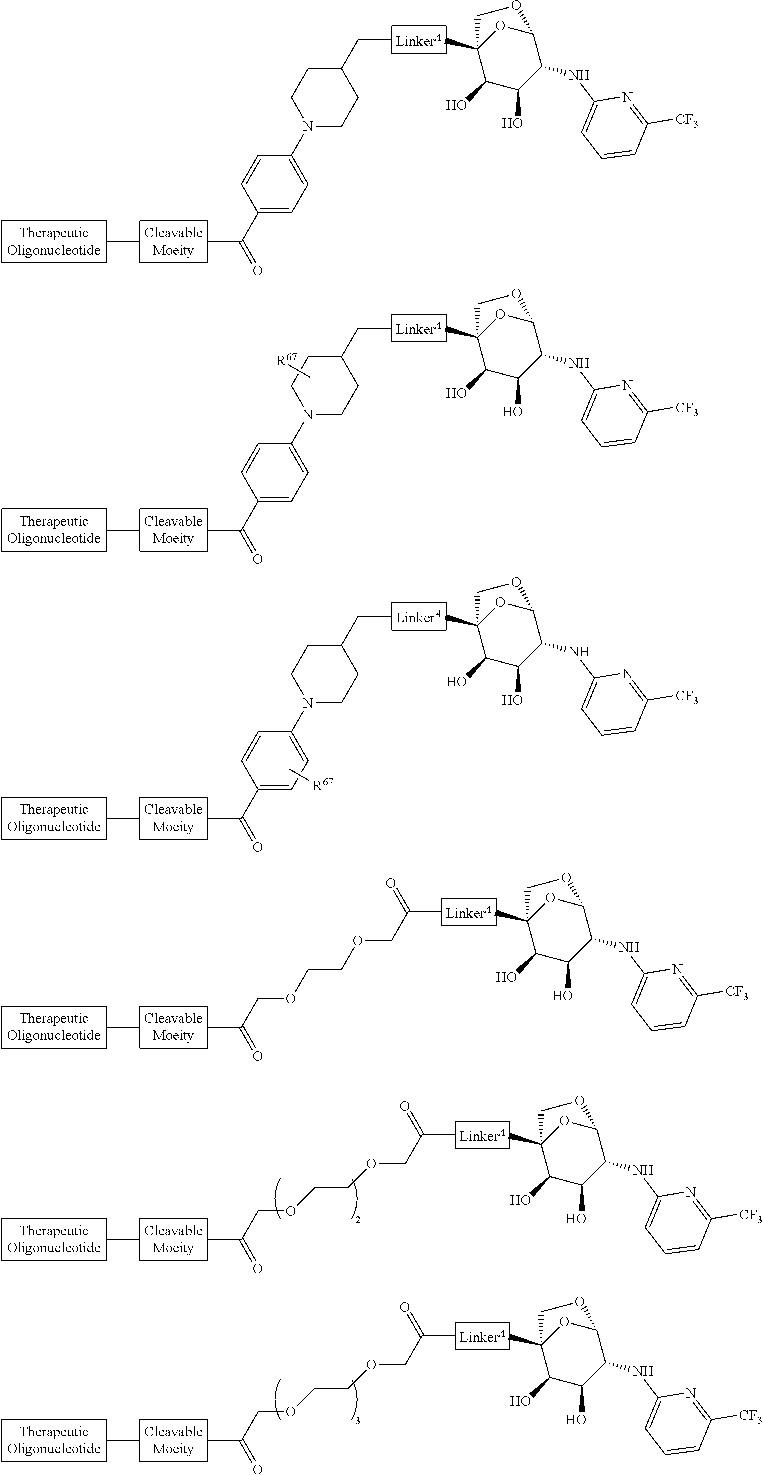

or a pharmaceutically acceptable salt thereof.

In certain embodiments the compound of the present invention is:

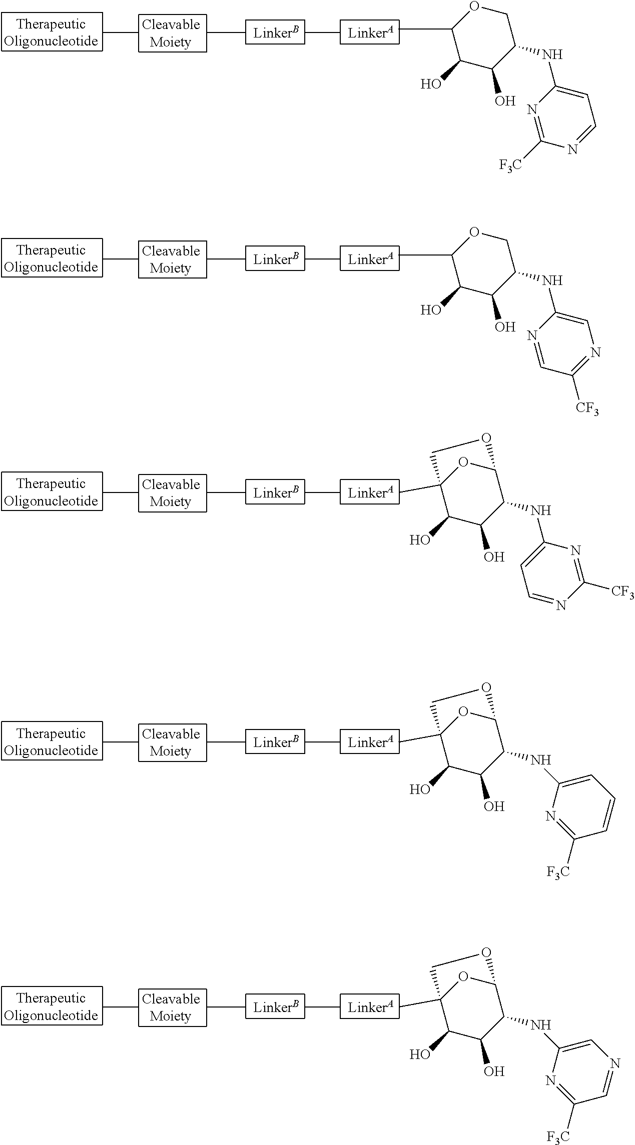

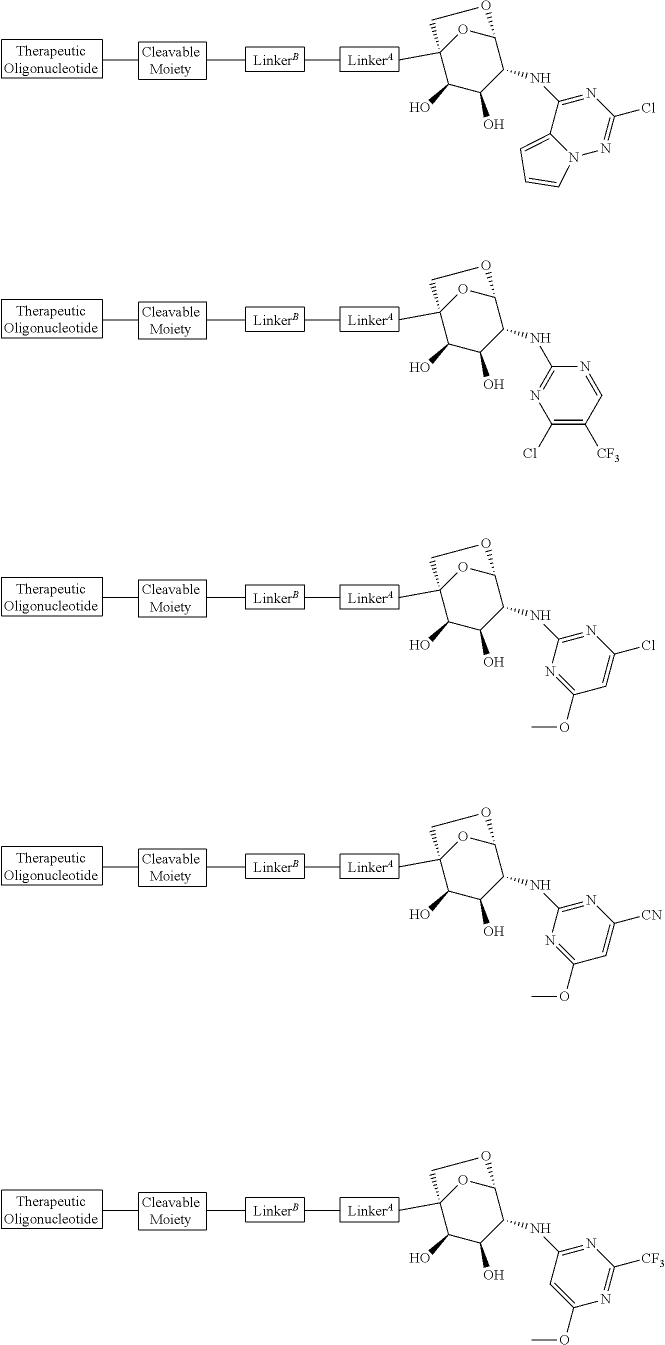

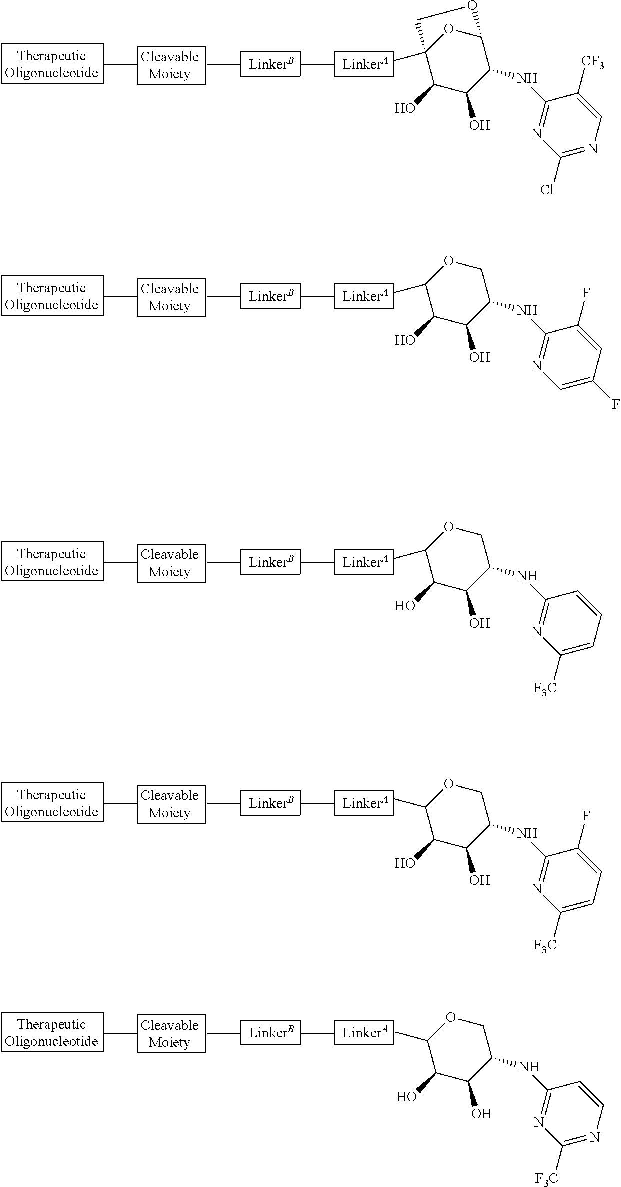

or a pharmaceutically acceptable salt thereof.

In certain aspects the invention provides a bidentate version of a monodentate embodiment presented herein. For example, in this aspect the bidentate of the following embodiment

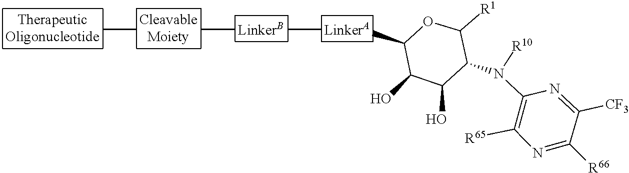

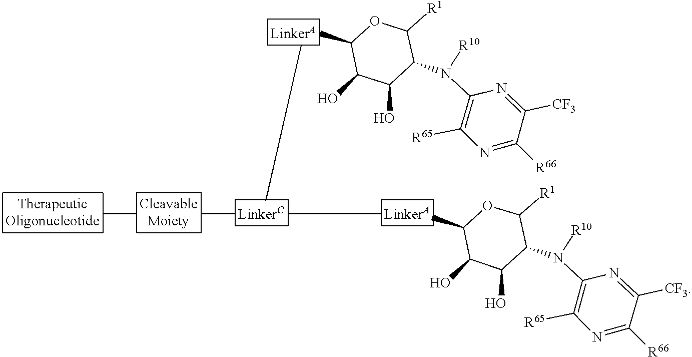

In certain aspects the invention provides a tridentate version of a monodentate embodiment presented herein. For example, in this aspect the tridentate of the following embodiment

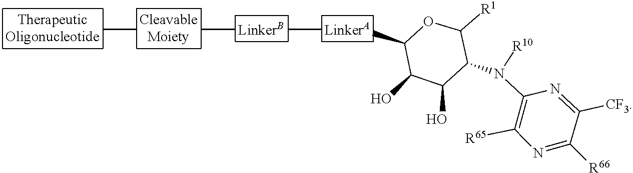

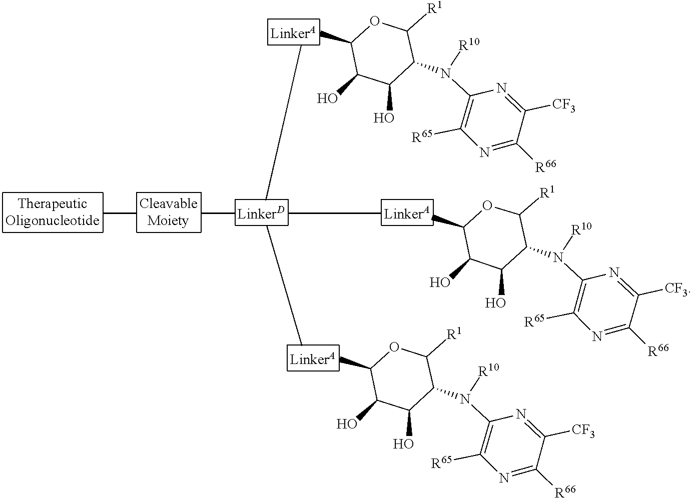

### Embodiments of the Linker

In non-limiting embodiments, Linker^{A} and Linker^{B} are independently selected from:

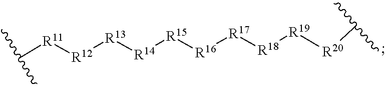

wherein:
R¹¹, R¹², R¹³, R¹⁴, R¹⁵, R¹⁶, R¹, R⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, and heterocycle;
and the remaining variables are as defined herein.

In one embodiment Linker^{A} is bond and Linker^{B} is

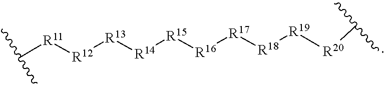

In one embodiment Linker^{B} is bond and Linker^{A} is

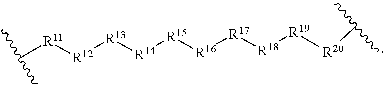

In certain embodiments, Linker^{A} is selected from

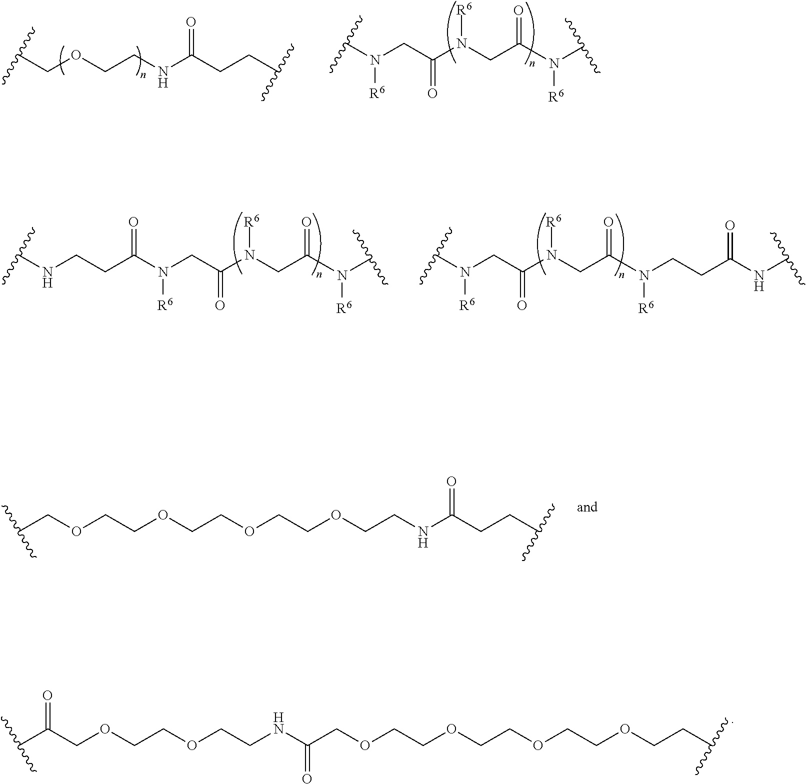

In certain embodiments

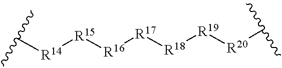

is selected from

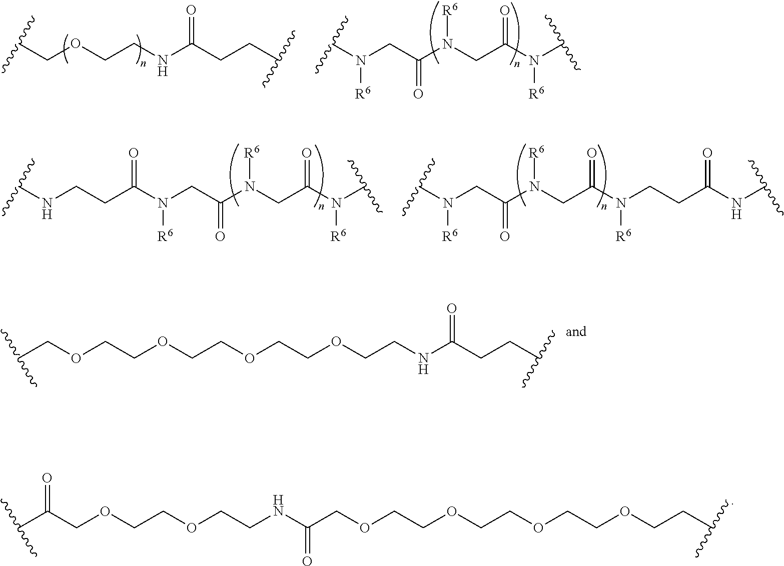

In the embodiments of R¹¹, R¹², R¹³, R¹⁴, R¹, R¹⁶, R¹⁷, R¹⁸, R¹⁹, and R²⁰ provided herein the structure can be included in the Linker as read from left to right or alternatively as read from right to left unless excluded by context. For example, when R¹⁵ is

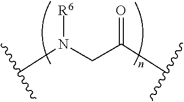

### and Linker^{B} is

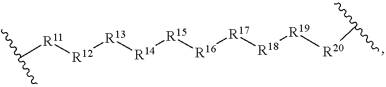

### Linker^{B} can be

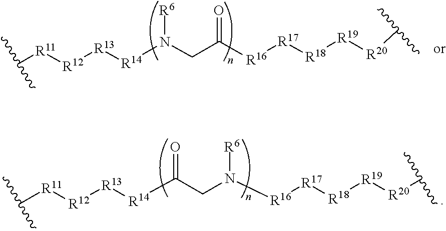

In certain embodiments, R¹¹, R¹², R¹³, R¹⁴, R¹⁵, R¹⁶, R¹⁷, R¹⁸, R¹⁹, and R²⁰ are each independently selected from the group consisting of bond, —C(O)—, —C(O)O—, —OC(O)—, —C(O)NR⁶—, —NR⁶C(O)—, —O—, —NR⁶—, —C(R²¹R²¹)—, a divalent residue of a natural or unnatural amino acid, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[—NR⁶C(R²¹R²¹)C(O)O—]ₓₓ—, and a divalent residue of a fatty acid; each of which is optionally substituted with 1, 2, 3, or 4 substituents independently selected from R²¹.

In certain embodiments, R¹¹, R¹², R¹³, R¹⁴, R¹⁵, R¹⁶, R¹⁷, R¹⁸, R¹⁹, and R²⁰ are each independently selected from the group consisting of bond, —O—, —NR—, —C(R²¹R²¹)—, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, and —[C(O)—CH₂—O]ₙ—; each of which is optionally substituted with 1, 2, 3, or 4 substituents independently selected from R²¹.

In certain embodiments, R¹¹, R¹², R¹³, R¹⁴, R¹⁵, R¹⁶, R¹⁷, R, R¹⁹, and R²⁰ are each independently selected from the group consisting of bond, —O—, —C(R²¹R²¹)—, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, and —[C(O)—CH₂—O]ₙ—; each of which is optionally substituted with 1, 2, 3, or 4 substituents independently selected from R²¹.

In certain embodiments, R¹¹, R¹², R¹³, R¹⁴, R¹⁵, R¹⁶, R¹⁷, R¹⁸, R¹⁹, and R²⁰ are each independently selected from the group consisting of bond, —C(O)—, —C(O)O—, —OC(O)—, —C(O)NR⁶, —NR⁶C(O)—, —O—, —NR⁶—, —C(R²¹R²¹)—, a divalent residue of a natural or unnatural amino acid, —CH₂CH₂—[O—(CH₂)₂]ₙ—NR⁶—, and —[—NR⁶C(R²¹R²¹)C(O)O—]ₓₓ—; each of which is optionally substituted with 1, 2, 3, or 4 substituents independently selected from R²¹.

In alternative embodiments, R¹¹, R¹², R¹³, R¹⁴, R¹⁵, R¹⁶, R¹⁷, R¹⁸, R¹⁹, and R²⁰ are each independently selected from the group consisting of bond, —C(O)—, —C(O)O—, —OC(O)—, —C(O)NR⁶, —NR⁶C(O)—, —O—, —NR⁶—, —C(R²¹R²¹)—, a divalent residue of a natural or unnatural amino acid, —CH₂CH₂—[O—(CH₂)₂]ₙ—NR⁶—, and —[—NR⁶C(R²¹R²¹)C(O)—]ₓₓ—; each of which is optionally substituted with 1, 2, 3, or 4 substituents independently selected from R²¹.

In one embodiment, a divalent residue of an amino acid is selected from

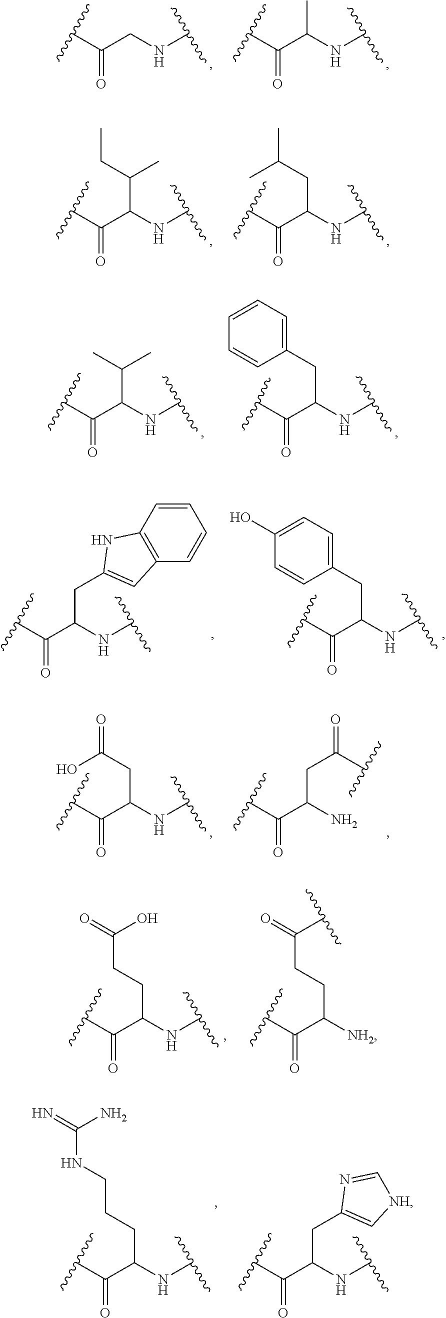

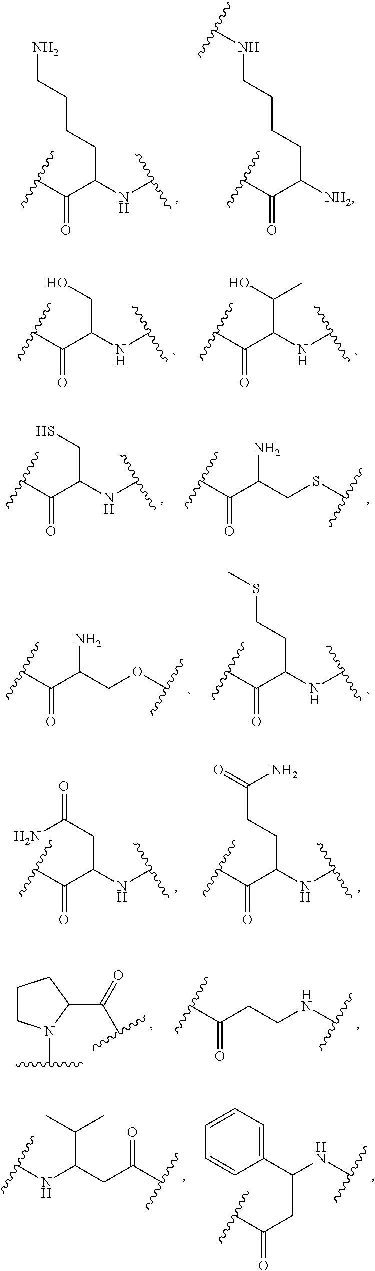

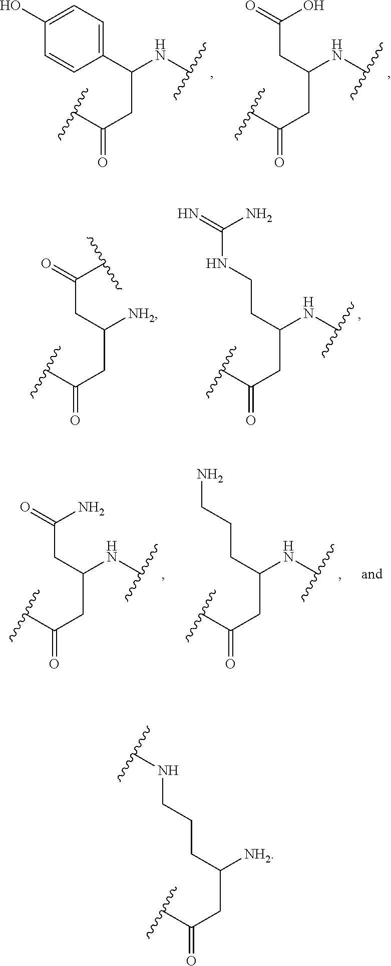

wherein the amino acid can be oriented in either direction and wherein the amino acid can be in the L- or D-form. For example, when R¹⁵ is

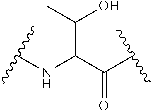 [SMILES: *NC(C(*)=O)C(C)O]

### and Linker^{B} is

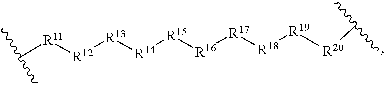

### Linker^{B} can be

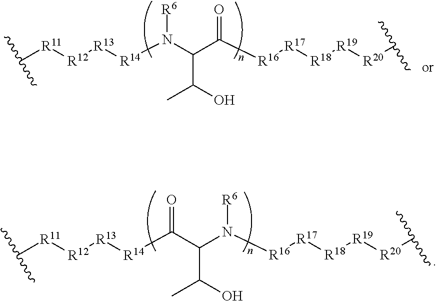

In certain embodiments, Linker^{A}, Linker^{B}, Linker^{C}, or Linker^{D} comprises a polymer linker. In certain embodiments, the polymer linker is a copolymer. In certain embodiments the polymer linker is a block copolymer linker. In certain embodiments, the polymer linker is an alternating copolymer linker. In certain embodiments, the polymer linker is a random copolymer.

In certain embodiments, the polymer linker is not branched. In certain embodiments, the polymer linker is branched. For example, a polymer comprising ethylene glycol monomers does not have a branch point. However, a polymer comprising propylene glycol monomers contains a branch point. In certain embodiments, a branch point is an R²¹ group.

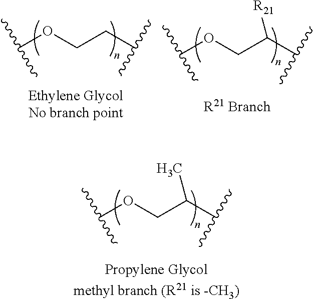

In certain embodiments, the polymer linker has one branch point. In certain embodiments, the polymer linker has two, three, four, five, six, seven, eight, nine, or ten branch points. In certain embodiments, the R¹¹, R¹², R¹³, R¹⁴, R¹⁵, R¹⁶, R¹⁷, R¹⁸, R¹⁹, or R²⁰ group is branched by one R²¹ group. In certain embodiments, the R¹¹, R¹², R¹³, R¹⁴, R¹⁵, R¹⁶, R¹⁷, R¹⁸, R¹⁹, or R²⁰ group is branched by two R²¹ groups. In certain embodiments, the R¹¹, R¹², R¹³, R¹⁴, R¹⁵, R¹⁶, R¹⁷, R¹⁸, R¹⁹, or R²⁰ group is branched by three R²¹ groups. In certain embodiments, the R¹¹, R¹², R¹³, R¹⁴, R¹⁵, R¹⁶, R¹⁷, R¹⁸, R¹⁹, or R²⁰ group is branched by four R²¹ groups.

For example, when R¹¹ is —[—(CH₂)₂—O—]ₙ—, the ethylene glycol monomer can be mono-, di-, or tribranched depending on the number of R²¹ substituents and regiochemistry of R²¹ substituents:

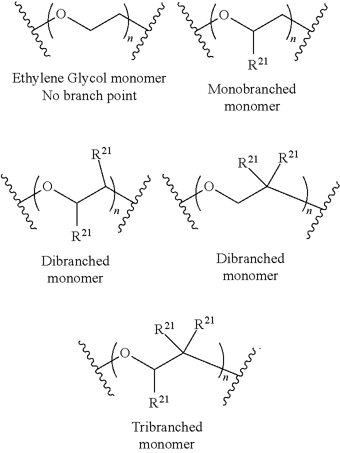

Linker monomers of different structures can be combined in a repeating or random pattern and can be oriented in either direction such that a stable compound results. For example, if R¹³, R¹⁵, R¹⁷ are —[—(CH₂)₂—O—]ₙ— substituted by one R²¹ group;
R¹⁴, R¹⁶, and R¹⁸ are —[—NR⁶C(R^{21a}R^{21b})C(O)—]ₓₓ—;
n is 3;
xx is 3;
R²¹ is CH₃,
R^{21a} and R^{2lb} are hydrogen; and
R⁶ is CH₃;
the alternating block copolymer linker would be of the formula:

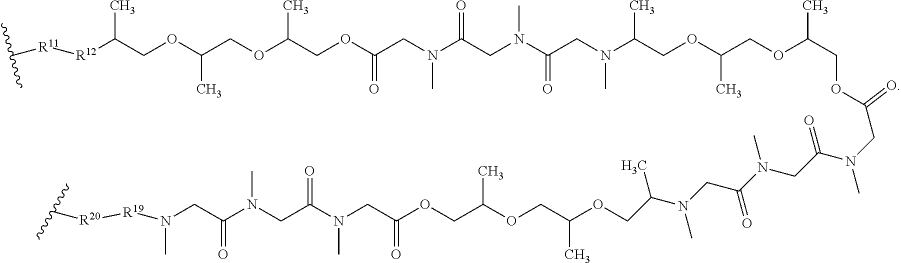

In certain embodiments, R¹¹, R¹², R¹³, R¹⁴, R¹, R¹⁶, R¹⁷, R¹⁸R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R^{21a}R^{21b})—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[—NR⁶C(R^{21a}R^{21b})C(O)O—]ₓₓ— a divalent residue of a fatty acid, a divalent residue of an unsaturated or saturated mono- or di-carboxylic acid,

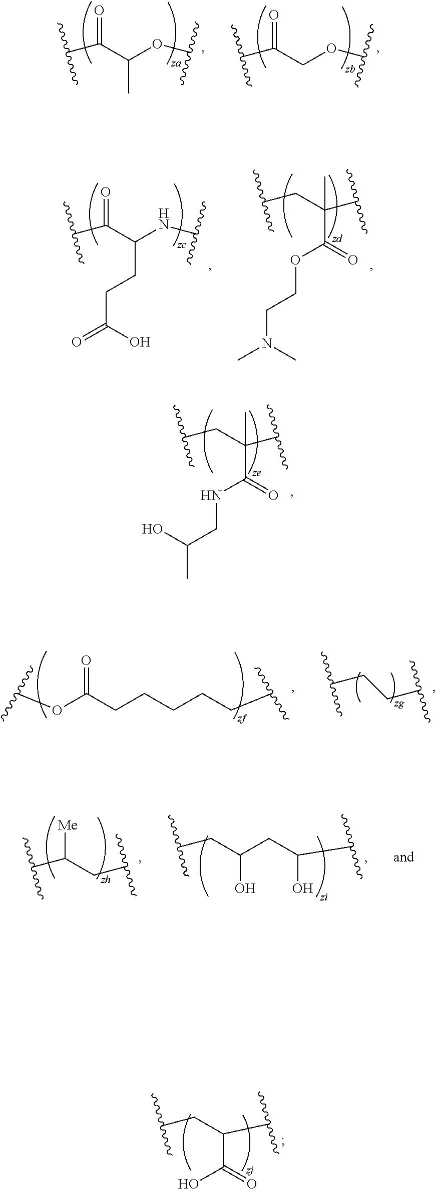

each of which is optionally substituted with 1, 2, 3, or 4 substituents independently selected from R²¹;
wherein za, zb, zc, zd, ze, zf, zg, zh, zi, and zj are independently selected at each instance from 0, 1, 2, 3, 4, 5, 6, 7, 8, 9, and 10, and wherein the sum of za, zb, zc, zd, ze, zf, zg, zh, zi, and zj is not more than 50.

In certain embodiments, the sum of the sum of za, zb, zc, zd, ze, zf, zg, zh, zi, and zj is not more than 30. In certain embodiments, the sum of za, zb, zc, zd, ze, zf, zg, zh, zi, and zj is not more than 25. In certain embodiments, the sum of za, zb, zc, zd, ze, zf, zg, zh, zi, and zj is not more than 15. In certain embodiments, the sum of za, zb, zc, zd, ze, zf, zg, zh, zi, and zj is not more than 10.

In alternative embodiments, R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R^{21a}R^{21b})—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[—NR⁶C(R^{21a}R^{21b})C(O)—]ₓₓ— a divalent residue of a fatty acid, a divalent residue of an unsaturated or saturated mono- or di-carboxylic acid,

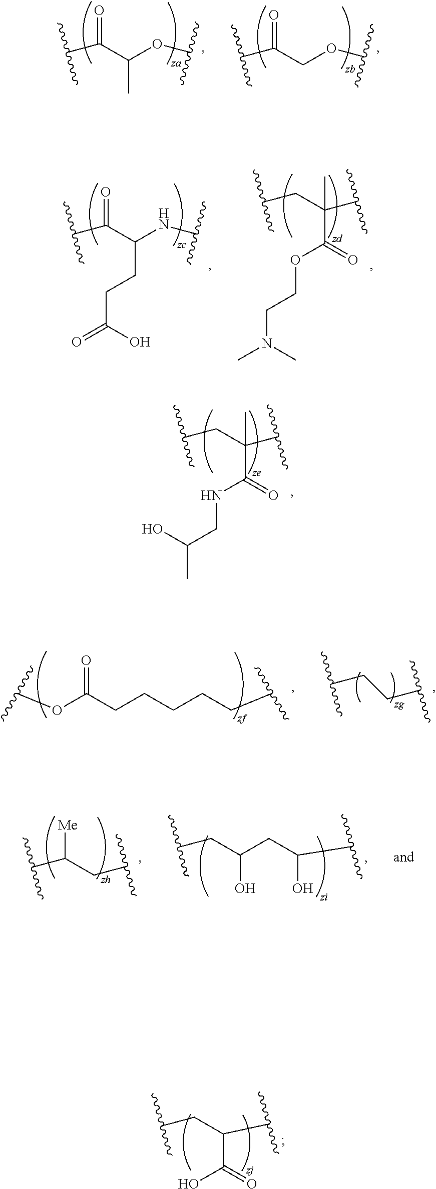

each of which is optionally substituted with 1, 2, 3, or 4 substituents independently selected from R²¹;

For example, a polymer linker can comprise an alternating block copolymer of the formula

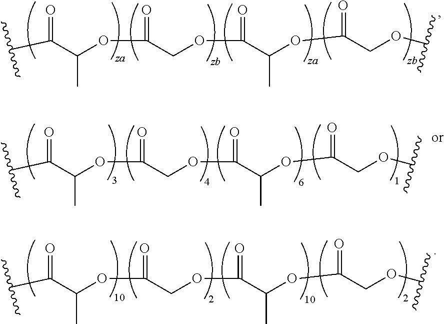

Linker monomers can be oriented in either direction. For example, when R¹ is

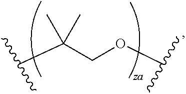

za is four, and Linker^{B} is

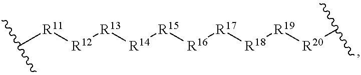

then Linker^{B} can be:

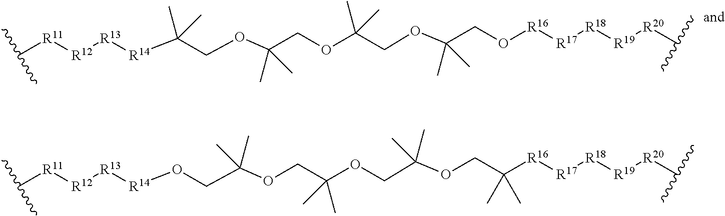

In certain embodiments, the polymer linker is a random copolymer. A random copolymer linker is a linker without a repeating pattern of R¹¹, R¹², R¹³, R¹⁴, R¹⁵, R¹⁶, R¹⁷, R¹⁸, R¹⁹, and R²⁰ monomer groups. In some embodiments, a random copolymer linker has different groups for each of R¹¹, R¹², R¹³, R¹⁴, R¹⁵, R¹⁶, R¹⁷, R¹⁸, R¹⁹, and R²⁰. In certain embodiments, two, three, four or five of R¹¹, R¹², R¹³, R¹⁴, R¹⁵, R¹⁶, R¹⁷, R¹⁸, R¹⁹, and R²⁰ in a random copolymer linker are the same group, but are present in a random order.

For example, a random block copolymer linker can comprise a polymer of the formula

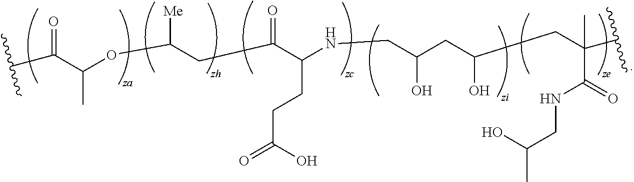

In certain embodiments, the random copolymer linker is a polymer linker comprised of up to 50, up to 30, up to 25, up to 20, or up to 15 randomly attached linker monomers selected from —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[—NR⁶C(R^{21a}R^{21b})C(O)O—]ₓₓ—,

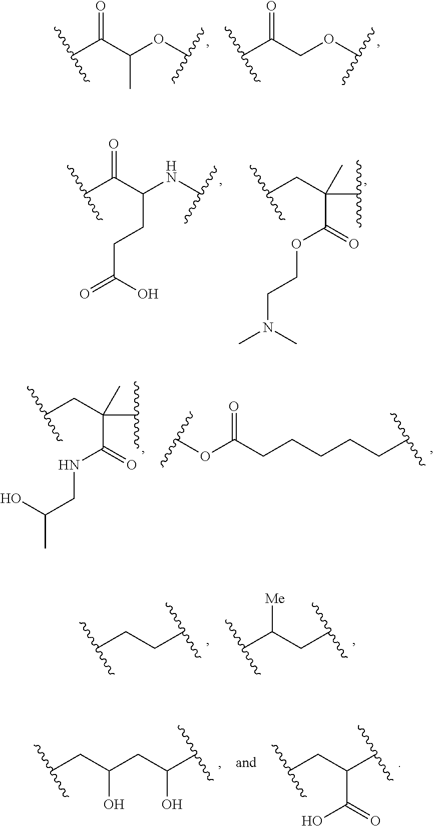

In certain embodiments, R¹¹, R¹², R¹³, R¹⁴, R¹⁵, R¹⁶, R¹⁷, R, R¹⁹, and R²⁰ are each independently selected from the group consisting of

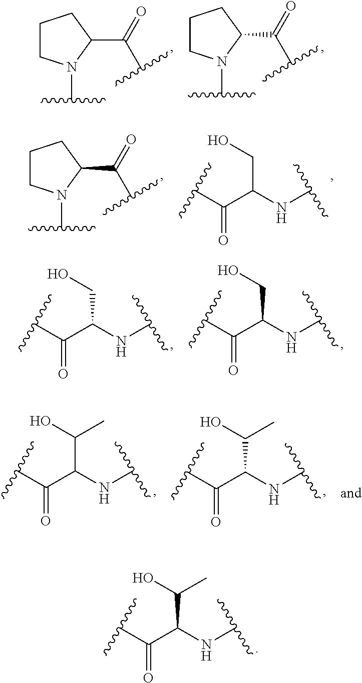

In the embodiments of R¹¹, R¹², R¹³, R¹⁴, R¹, R¹⁶, R¹⁷, R¹⁸, R¹⁹, and R²⁰ provided herein the structure can be included in the Linker as read from left to right or alternatively as read from right to left unless excluded by context. For example, when R¹⁵ is

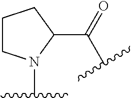 [SMILES: *C(=O)C1CCCN1*]

### and Linker^{B} is

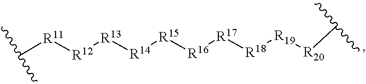

### Linker^{B} can be

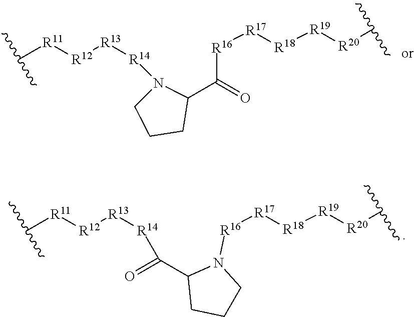

In certain embodiments, R¹¹, R¹², R¹³, R¹⁴, R¹⁵, R¹⁶, R¹⁷, R¹⁸, R¹⁹, and R²⁰ are each independently selected from the group consisting of

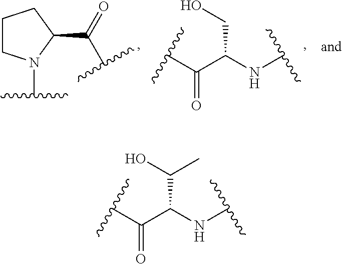

In certain embodiments, R¹¹, R¹², R¹³, R¹⁴, R¹⁵, R¹⁶, R¹⁷, R¹⁸, R¹⁹, and R²⁰ are each independently selected from the group consisting of

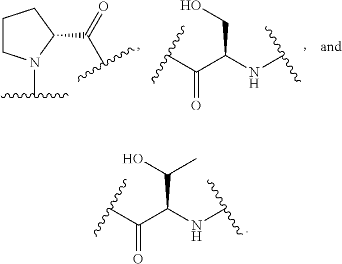

In certain embodiments, at least three of R¹¹, R¹², R¹³, R¹⁴, R¹⁵, R¹⁶, R¹⁷, R¹⁸, R¹⁹, and R²⁰ are each independently selected from the group consisting of

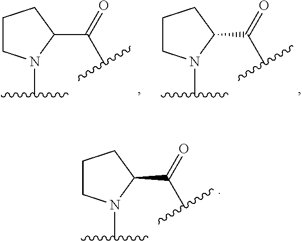 [SMILES: *C(=O)C1CCCN1* ; *C(=O)[C@H]1CCCN1* ; *C(=O)[C@@H]1CCCN1*]

In certain embodiments, at least four of R¹¹, R¹², R¹³, R¹⁴, R¹⁵, R¹⁶, R¹⁷, R¹⁸, R¹⁹, and R²⁰ are each independently selected from the group consisting of

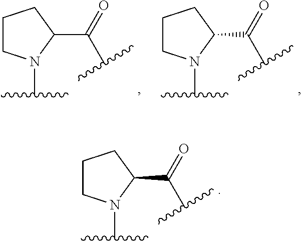 [SMILES: *C(=O)C1CCCN1* ; *C(=O)[C@H]1CCCN1* ; *C(=O)[C@@H]1CCCN1*]

In certain embodiments, at least five of R¹¹, R¹², R¹³, R¹⁴, R¹⁵, R¹⁶, R¹⁷, R¹⁸, R¹⁹, and R²⁰ are each independently selected from the group consisting of

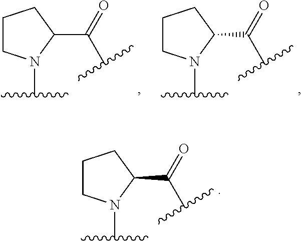 [SMILES: *C(=O)C1CCCN1* ; *C(=O)[C@H]1CCCN1* ; *C(=O)[C@@H]1CCCN1*]

In certain embodiments, at least six of R¹¹, R¹², R¹³, R¹⁴, R¹⁵, R¹⁶, R¹⁷, R¹⁸, R¹⁹, and R²⁰ are each independently selected from the group consisting of

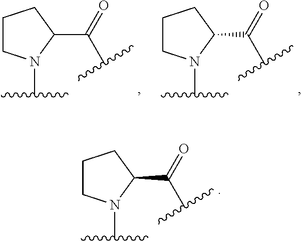 [SMILES: *C(=O)C1CCCN1* ; *C(=O)[C@H]1CCCN1* ; *C(=O)[C@@H]1CCCN1*]

In certain embodiments, at least seven of R¹¹, R¹², R¹³, R¹⁴, R, R¹⁶, R¹⁷, R¹⁸, R¹⁹, and R²⁰ are each independently selected from the group consisting of

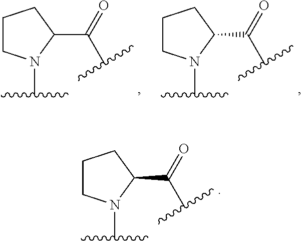 [SMILES: *C(=O)C1CCCN1* ; *C(=O)[C@H]1CCCN1* ; *C(=O)[C@@H]1CCCN1*]

In certain embodiments, at least eight of R¹¹, R¹², R¹³, R¹⁴, R¹⁵, R¹⁶, R¹⁷, R¹⁸, R¹⁹, and R²⁰ are each independently selected from the group consisting of

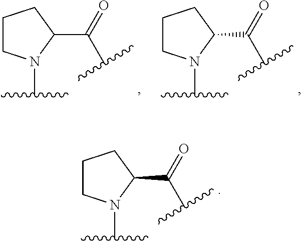 [SMILES: *C(=O)C1CCCN1* ; *C(=O)[C@H]1CCCN1* ; *C(=O)[C@@H]1CCCN1*]

In certain embodiments, at least nine of R¹¹, R¹², R¹³, R¹⁴, R¹⁵, R¹⁶, R¹⁷, R¹⁸, R¹⁹, and R²⁰ are each independently selected from the group consisting of

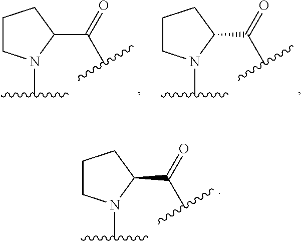 [SMILES: *C(=O)C1CCCN1* ; *C(=O)[C@H]1CCCN1* ; *C(=O)[C@@H]1CCCN1*]

In certain embodiments, Linker^{B} is selected from:

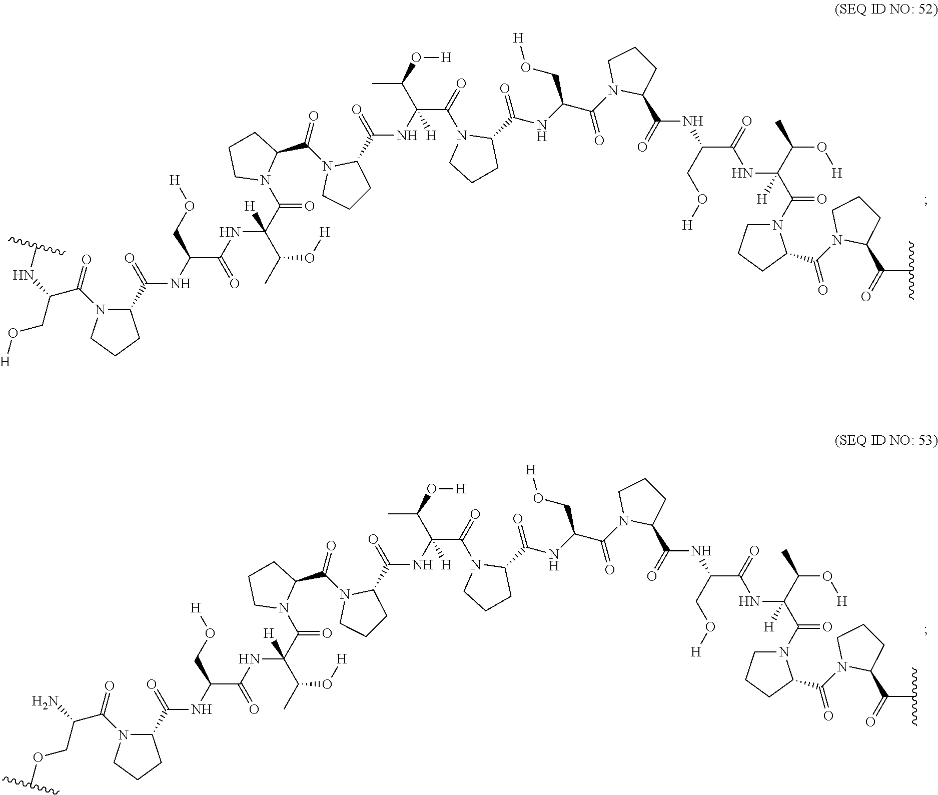 [SMILES: *N[C@@H](CO)C(=O)N1CCC[C@H]1C(=O)N[C@@H](CO)C(=O)N[C@H](C(=O)N1CCC[C@H]1C(=O)N1CCC[C@H]1C(=O)N[C@H](C(=O)N1CCC[C@H]1C(=O)N[C@@H](CO)C(=O)N1CCC[C@H]1C(=O)N[C@@H](CO)C(=O)N[C@H](C(=O)N1CCC[C@H]1C(=O)N1CCC[C@H]1C(*)=O)[C@@H](C)O)[C@@H](C)O)[C@@H](C)O ; *OC[C@H](N)C(=O)N1CCC[C@H]1C(=O)N[C@@H](CO)C(=O)N[C@H](C(=O)N1CCC[C@H]1C(=O)N1CCC[C@H]1C(=O)N[C@H](C(=O)N1CCC[C@H]1C(=O)N[C@@H](CO)C(=O)N1CCC[C@H]1C(=O)N[C@@H](CO)C(=O)N[C@H](C(=O)N1CCC[C@H]1C(=O)N1CCC[C@H]1C(*)=O)[C@@H](C)O)[C@@H](C)O)[C@@H](C)O]

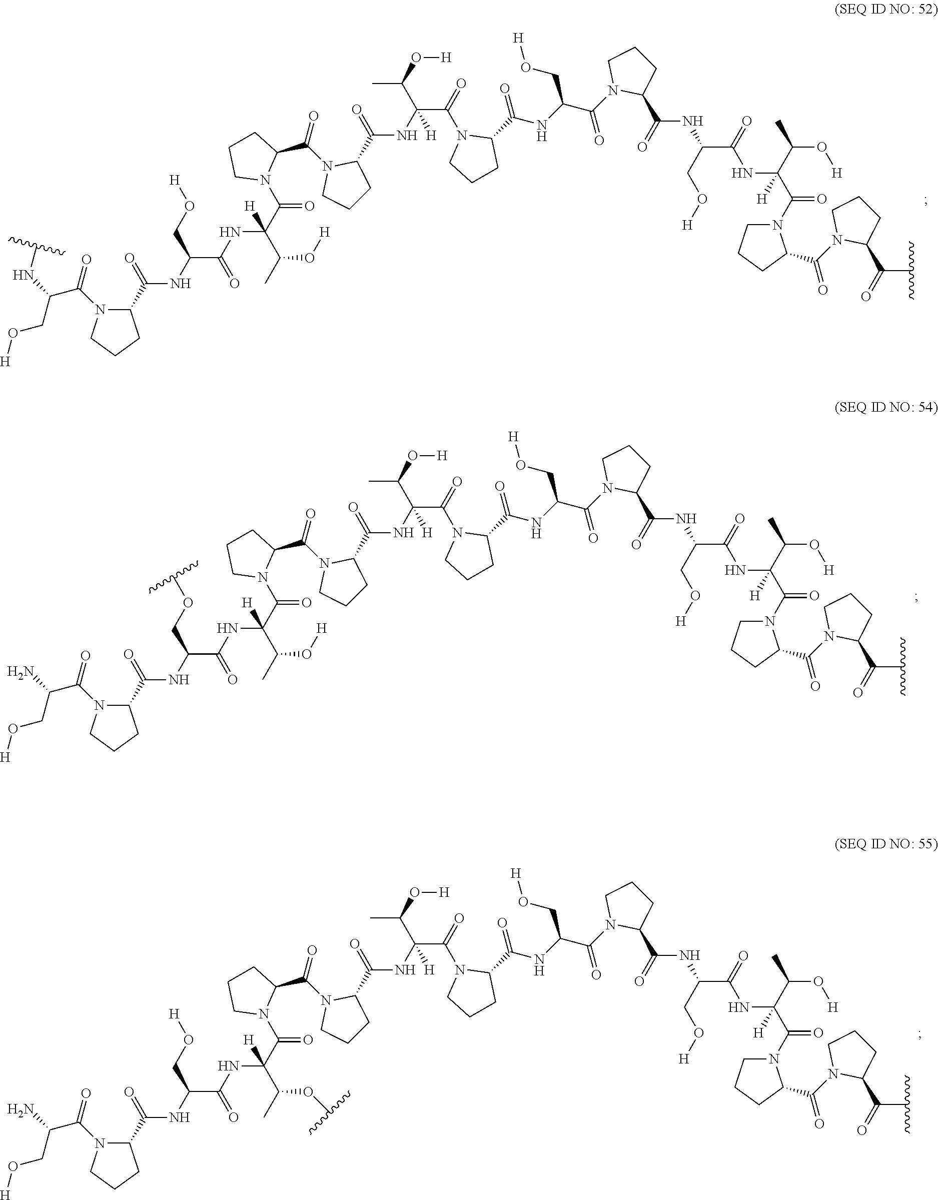

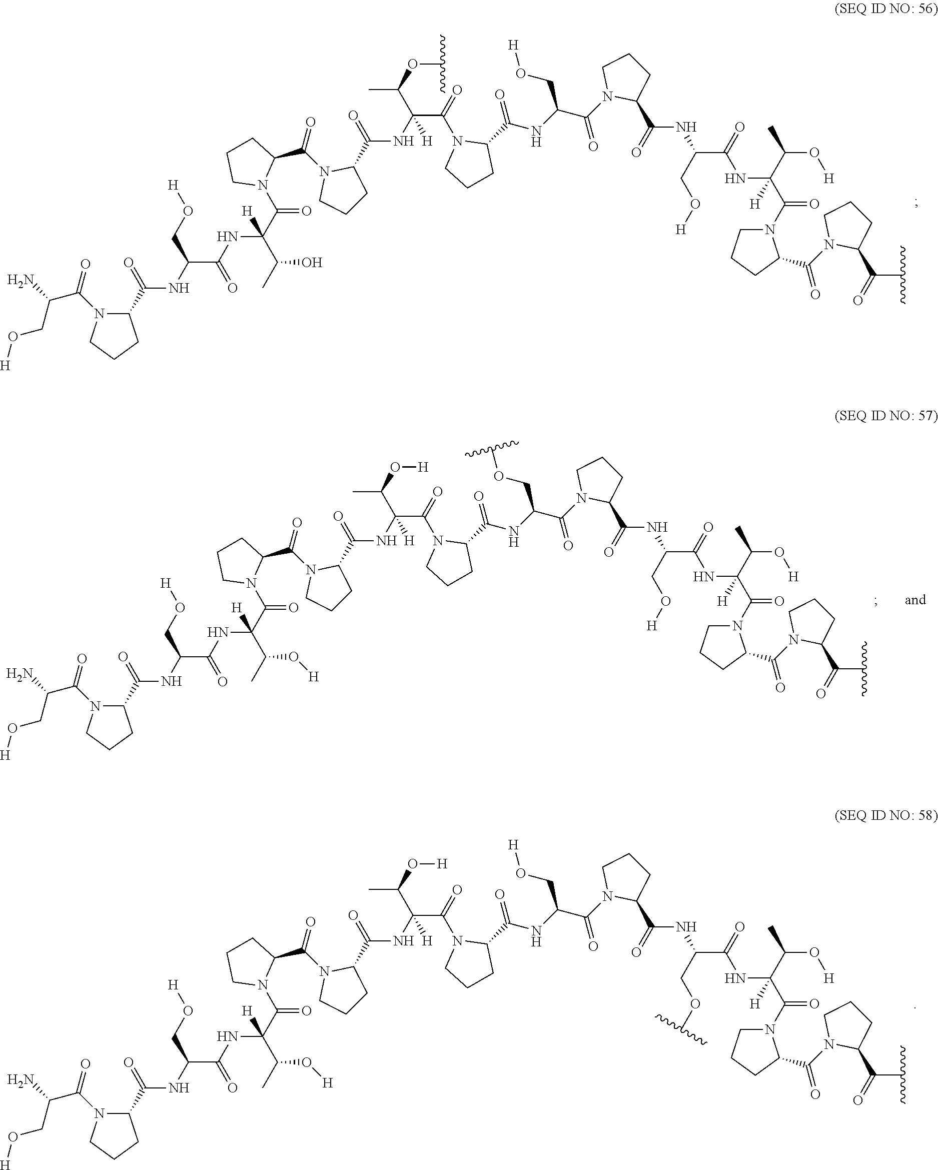

In certain embodiments, the Linker is a sequence comprising SEQ ID NO: 1, 2, 3, 4, 5, 6, 7, 8, 9, or 10; or a sequence that has at least 80%, 85%, 90%, 95%, or 98% sequence identity to SEQ ID NO: 1, 2, 3, 4, 5, 6, 7, 8, 9, or 10.

SEQ ID NO: 1
SPSTPPTPSPSTPP
SEQ ID NO: 2
SPSTPPTPSPSTPPSPSTPP
SEQ ID NO: 3
SPTSPPSPTPTSPP
SEQ ID NO: 4
PPTSPSPTPPTSPS
SEQ ID NO: 5
PPTSPSPTPPTSPSPP
SEQ ID NO: 6
SPPSTPSPTPTSPP
SEQ ID NO: 7
SPSTPSTPSPSTPP
SEQ ID NO: 8
PPTSPSPTPPTSPSSPSTPP
SEQ ID NO: 9
PPSTPTPSPPSTPS
SEQ ID NO: 10
PPSTPTPSPPSTPSPP

In certain embodiments, Linker is an amino acid sequence comprising a peptide that contains between 1 and 10 prolines and optionally between 1 and 10 amino acids selected from serine and threonine.

In one embodiment, a divalent residue of a dicarboxylic acid is generated from a nucleophilic addition reaction:

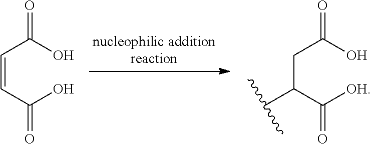 [SMILES: O=C(O)/C=C\C(=O)O ; *C(CC(=O)O)C(=O)O]

Non-limiting embodiments of a divalent residue of a dicarboxylic acid generated from a nucleophilic addition reaction include:

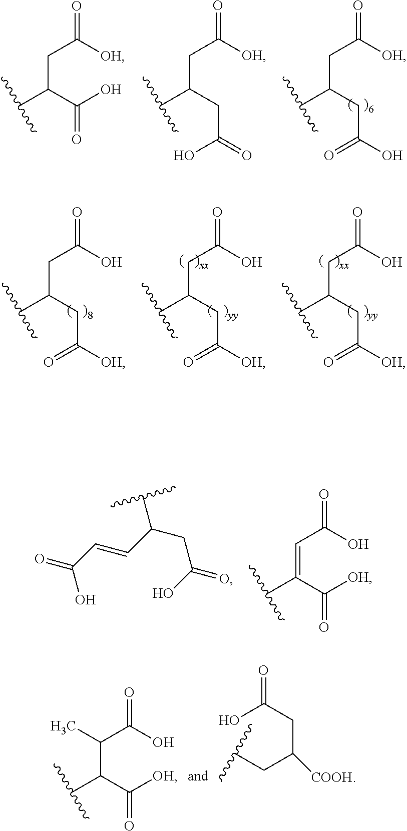

As used in the embodiments herein, xx is independently selected from 0, 1, 2, 3, 4, 5, 6, 7, 8, 9, 10, 11, 12, 13, 14, 15, 16, 17, 18, 19, 20, 21, 22, 23, 24, and 25.

As used in the embodiments herein, yy is independently selected from 0, 1, 2, 3, 4, 5, 6, 7, 8, 9, 10, 11, 12, 13, 14, 15, 16, 17, 18, 19, 20, 21, 22, 23, 24, and 25.

In one embodiment, a divalent residue of a dicarboxylic acid is generated from a condensation reaction:

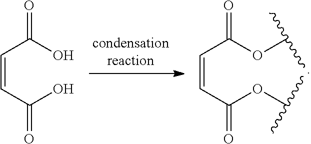 [SMILES: O=C(O)/C=C\C(=O)O ; *OC(=O)/C=C\C(=O)O*]

Non-limiting embodiments of a divalent residue of a dicarboxylic acid generated from a condensation include:

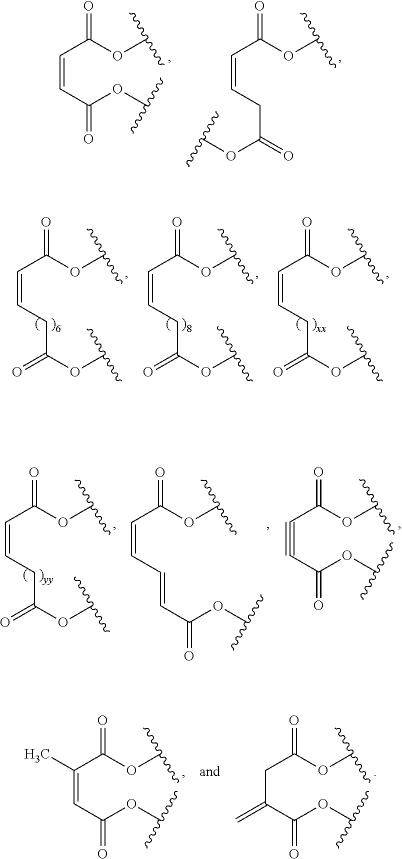

Non-limiting embodiments of a divalent residue of a saturated dicarboxylic acid include:

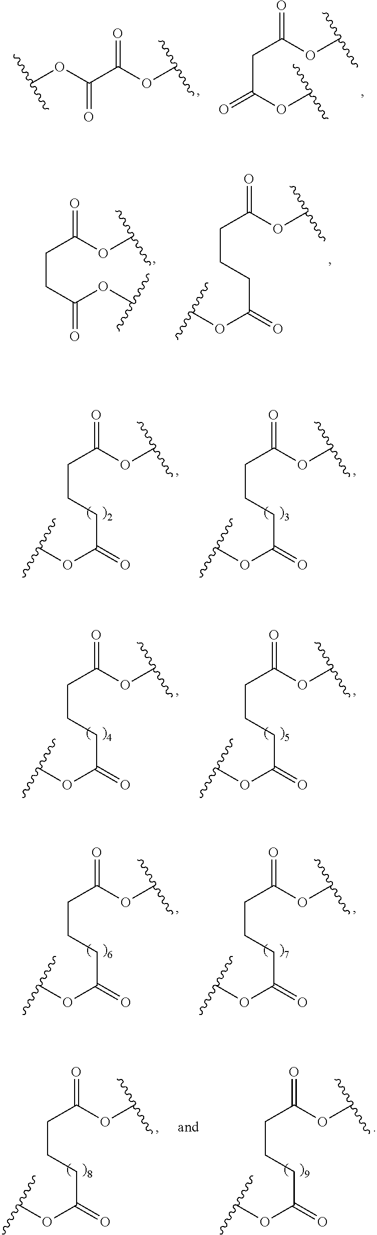

Non-limiting embodiments of a divalent residue of a saturated dicarboxylic acid include:

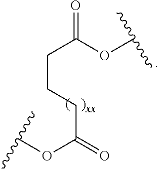

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂OCH₂—), and lignoceric acid (—OC(O)(CH₂)₂₂CH₂—).

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

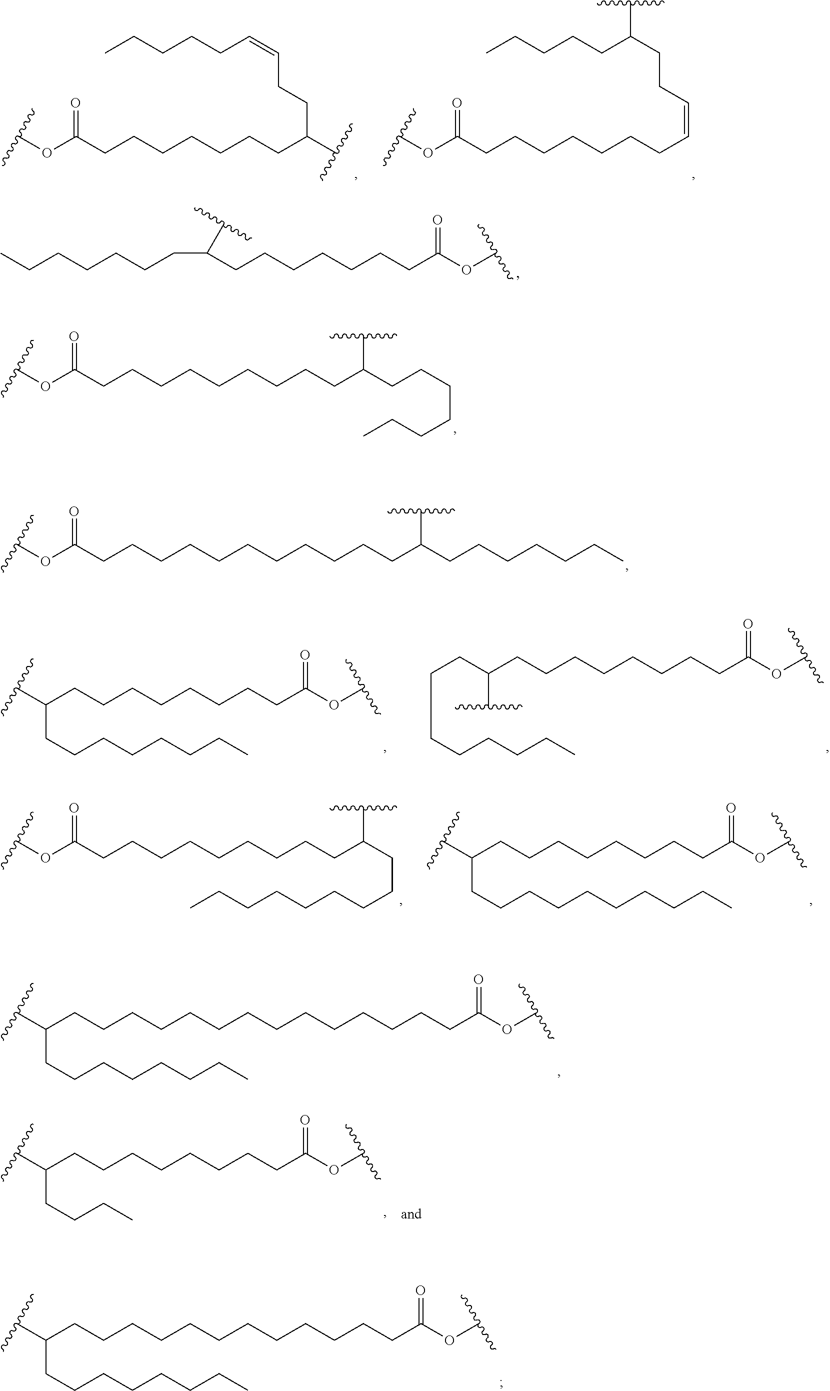

Non-limiting embodiments of a divalent residue of a fatty acid is selected from linoleic acid (—C(O)(CH₂)₇(CH)₂CH₂(CH)₂(CH₂)₄CH₂—), docosahexaenoic acid (—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid (—C(O)(CH₂)₄(CHCHCH₂)₄CH₂—), y-linolenic acid (—C(O)(CH₂)₄(CHCHCH₂)₃(CH₂)₃CH₂—), arachidonic acid (—C(O)(CH₂)₃,(CHCHCH₂)₄(CH₂)₄CH₂—), docosatetraenoic acid (—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₉CHCH(CH₂)₅CH₂—), paullinic acid (—C(O)(CH₂)₁₁CHCH(CH₂)₅CH₂—), oleic acid (—C(O)(CH₂)₇CHCH(CH₂)₇CH₂—), elaidic acid (—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₇CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments Linker^{C} is selected from:

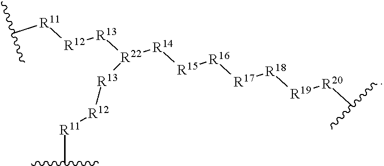

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments Linker^{D} is selected from:

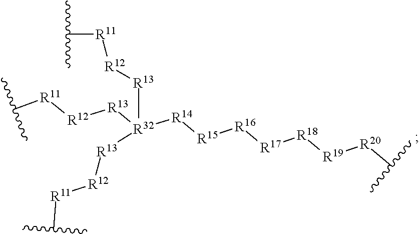

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻; and
all other variables are as defined herein.

In certain embodiments, Linker^{C} is selected from

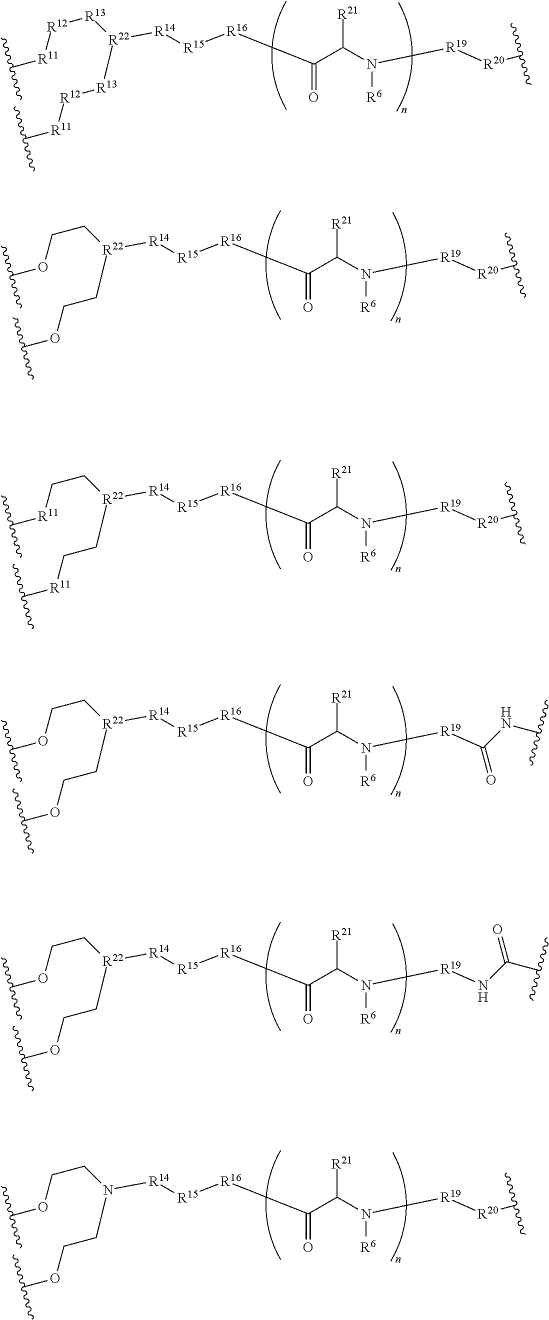

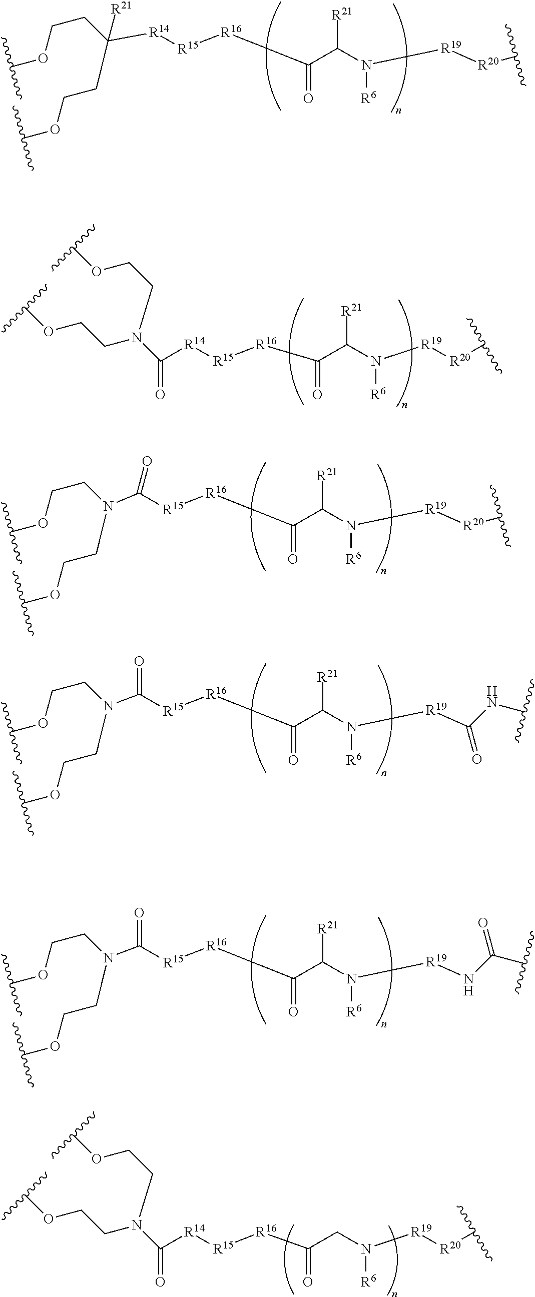

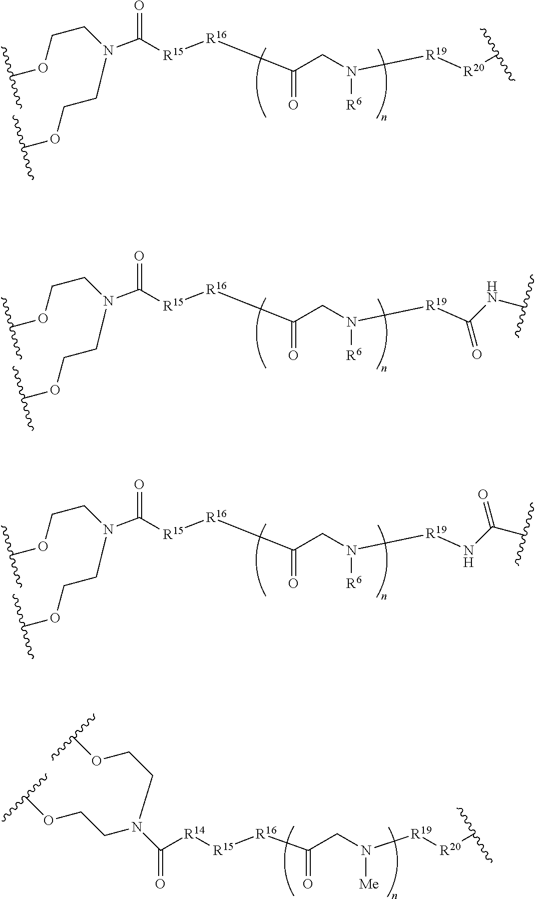

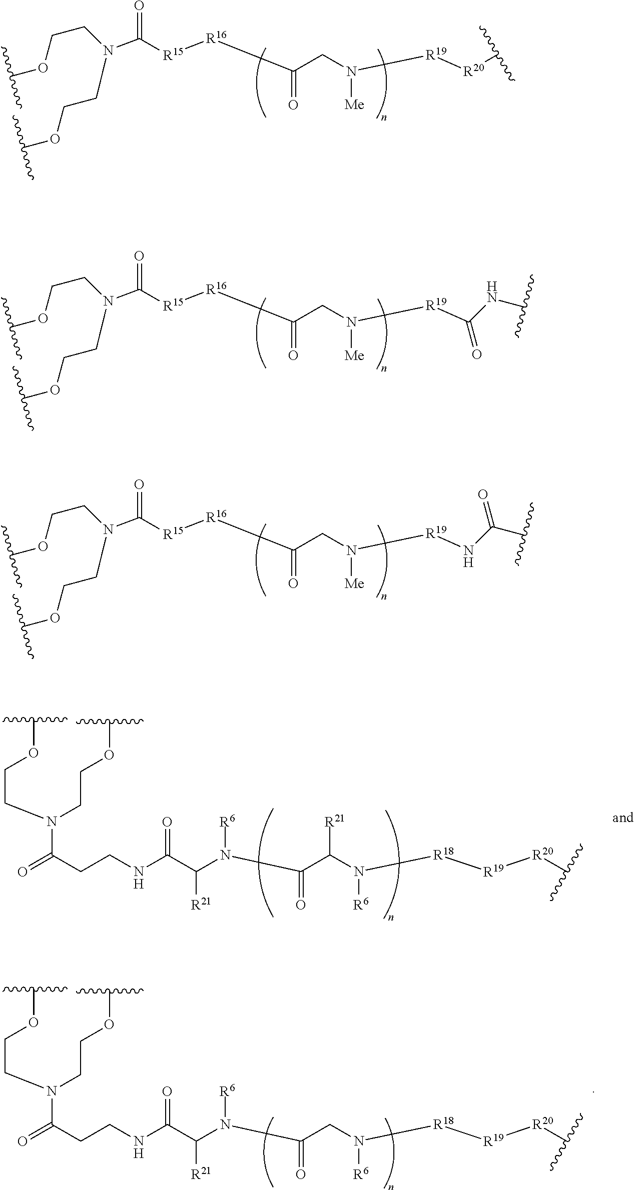

In certain embodiments, Linker^{C} is selected from

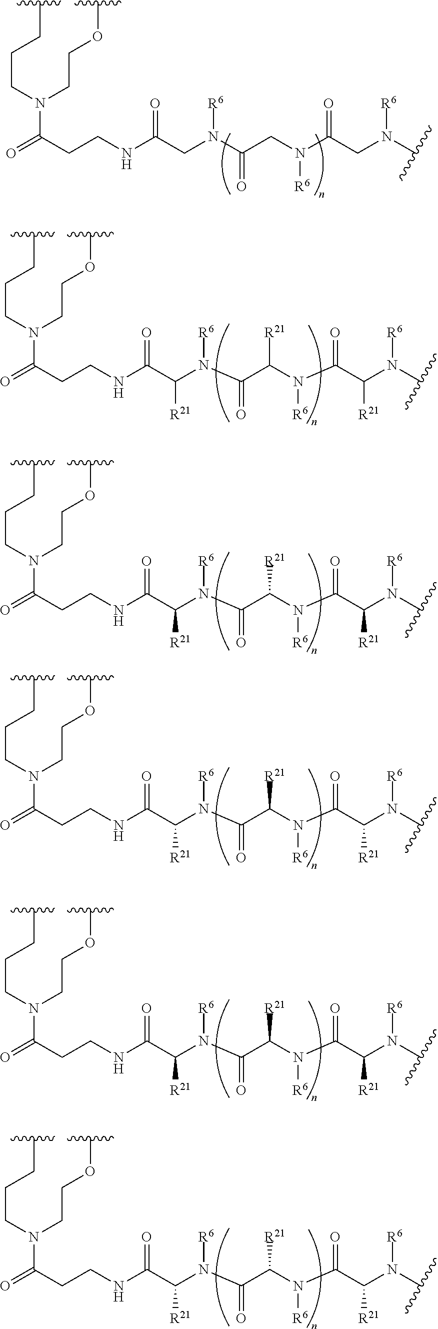

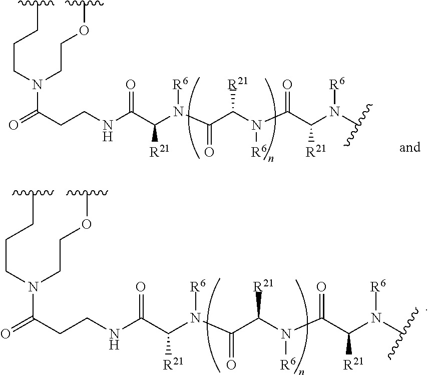

In certain embodiments, Linker^{C} is selected from

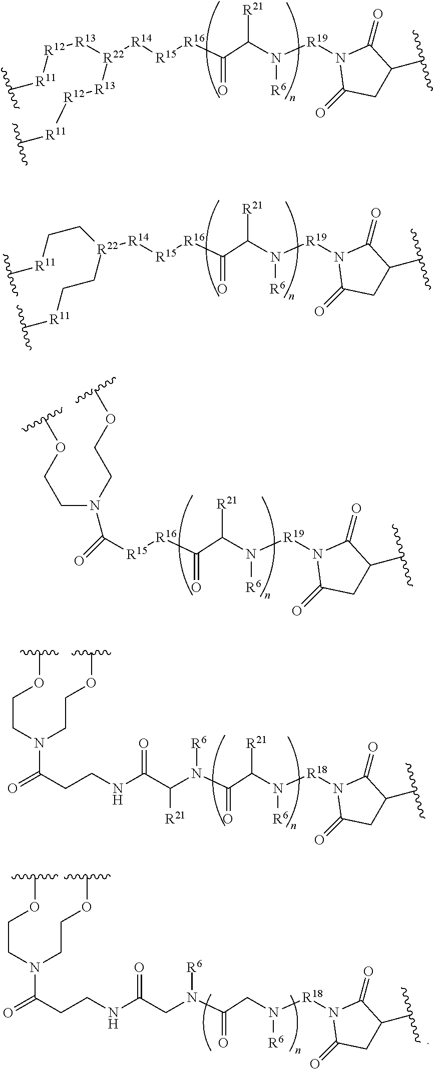

In certain embodiments, Linker^{C} is selected from

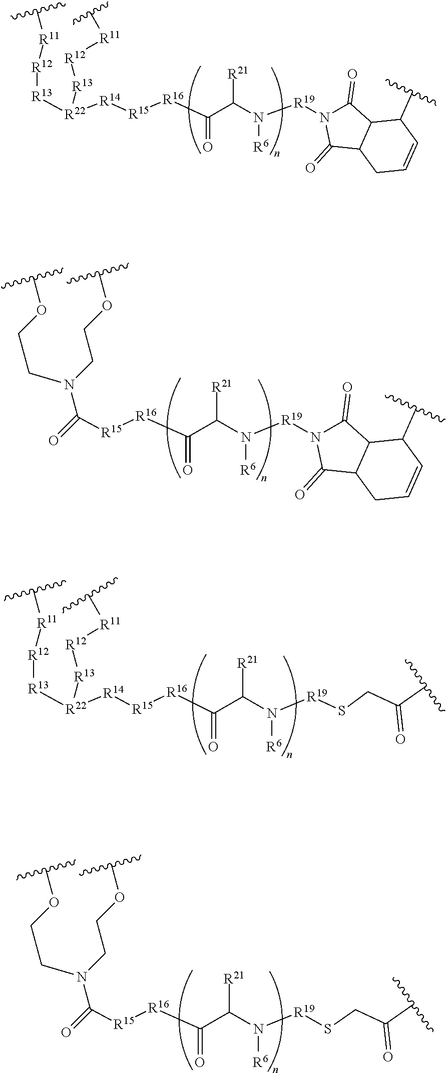

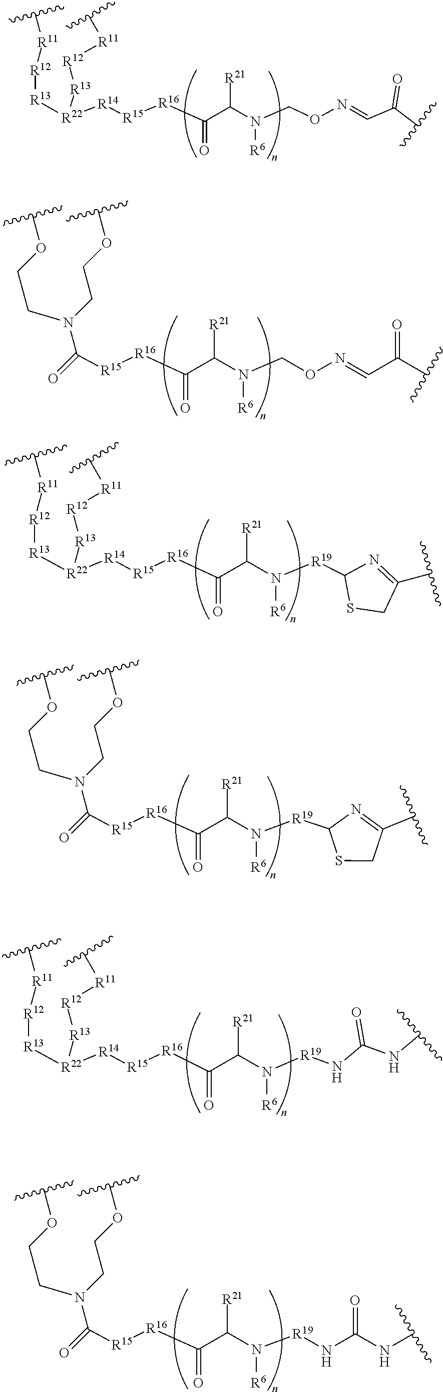

In certain embodiments, Linker^{C} is selected from

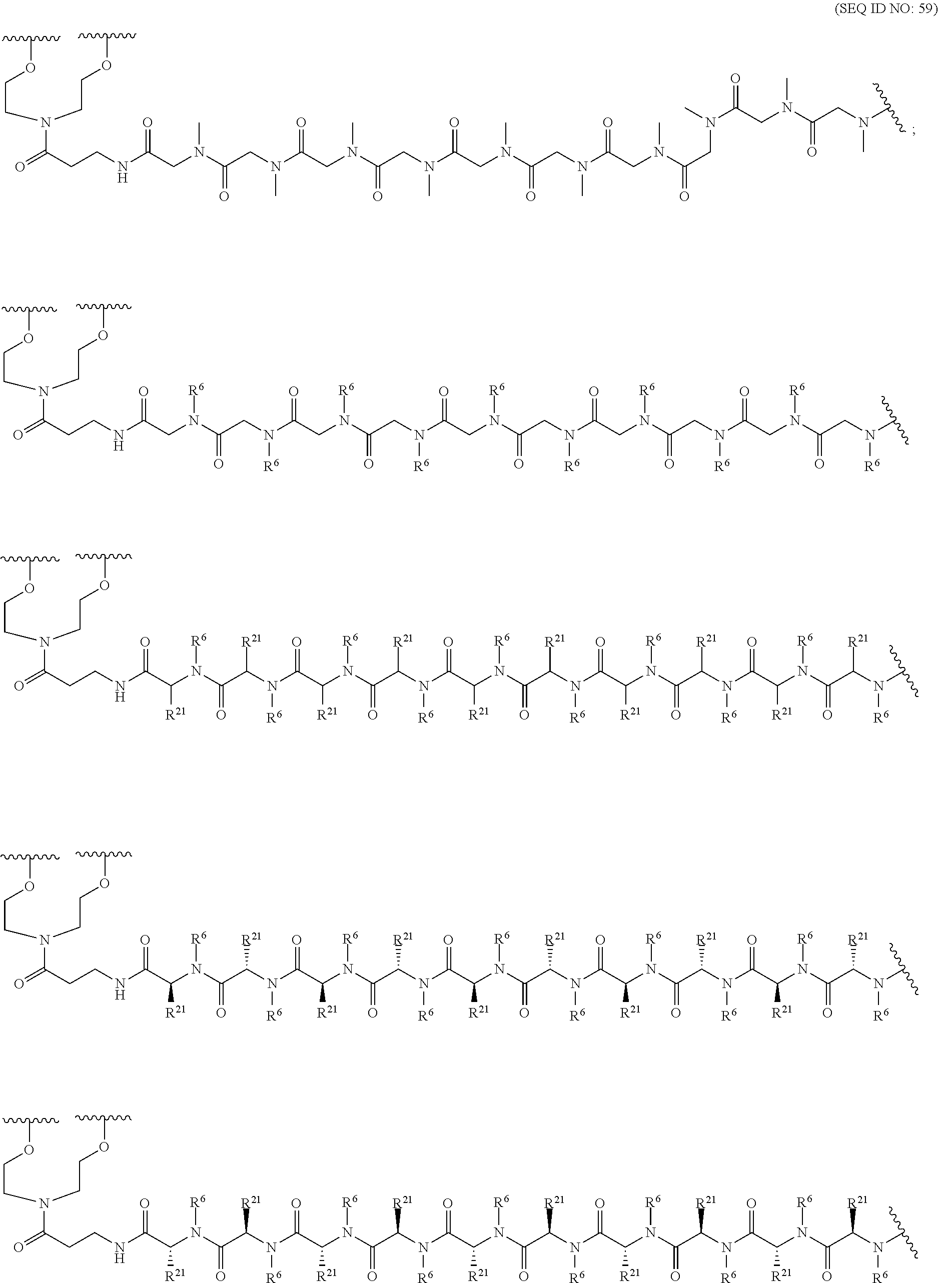

In certain embodiments, Linker^{C} is selected from:

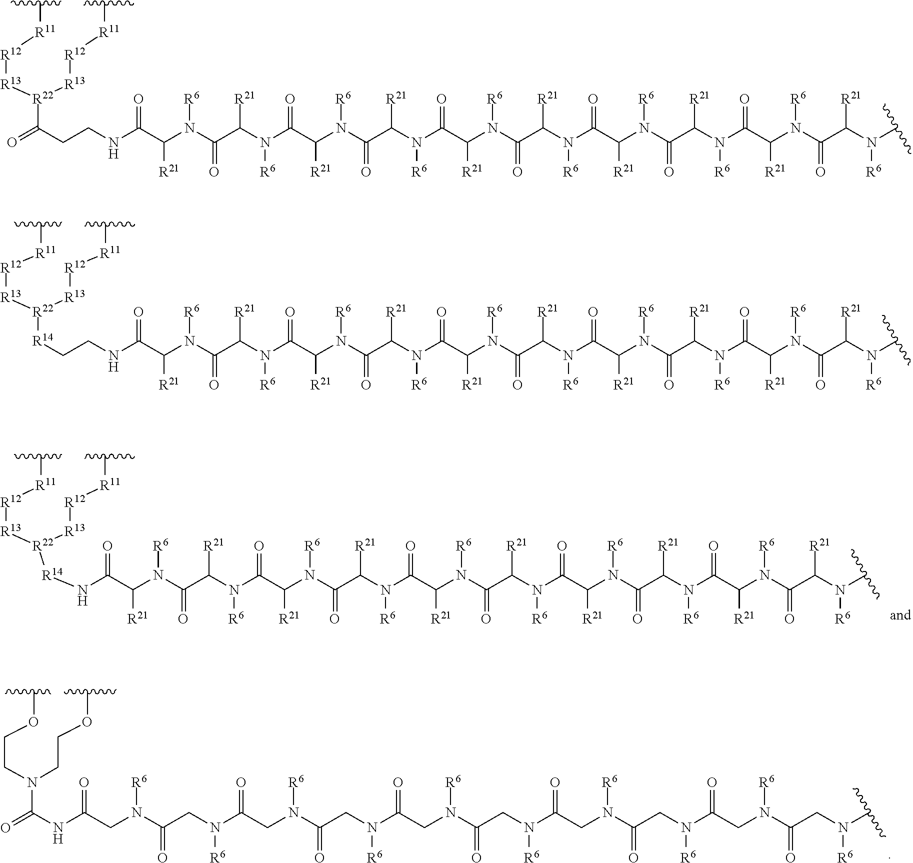

In certain embodiments, Linker^{C} is selected from:

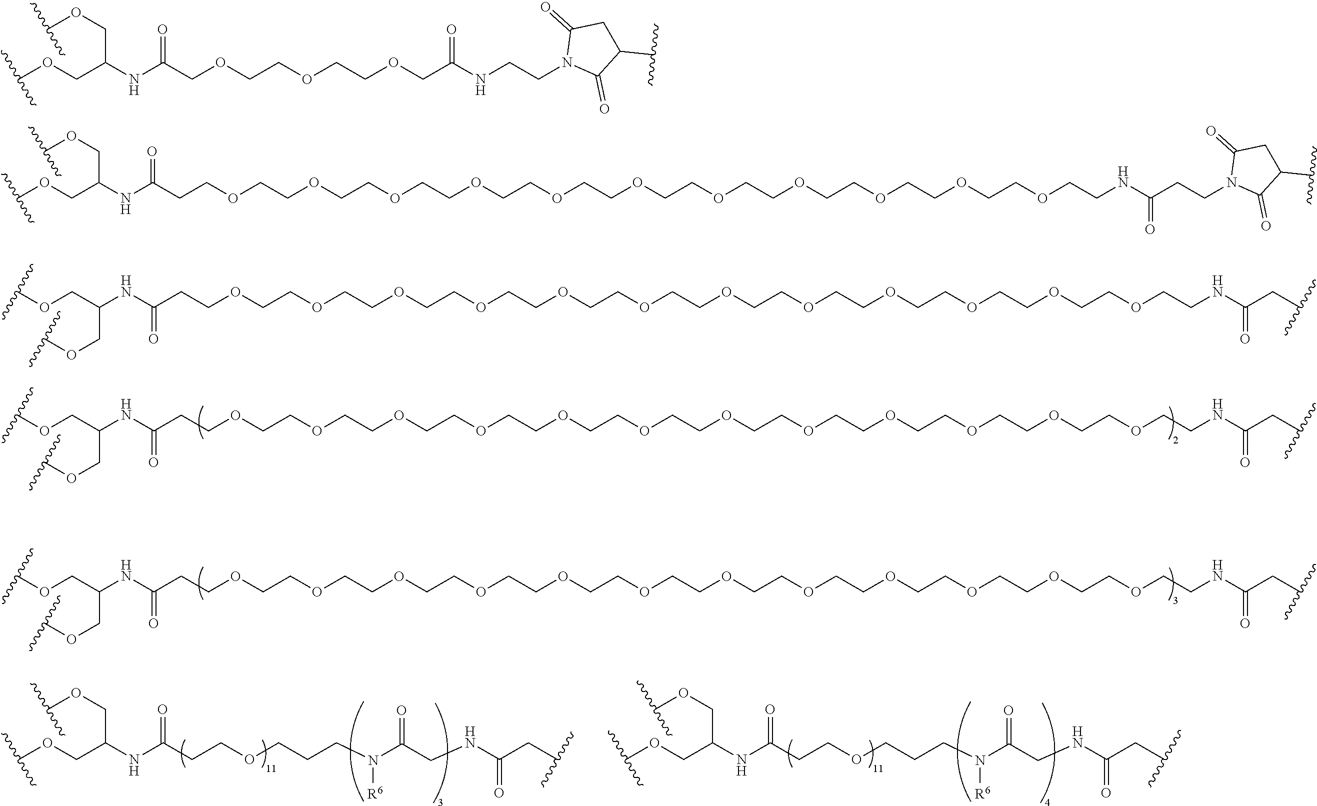

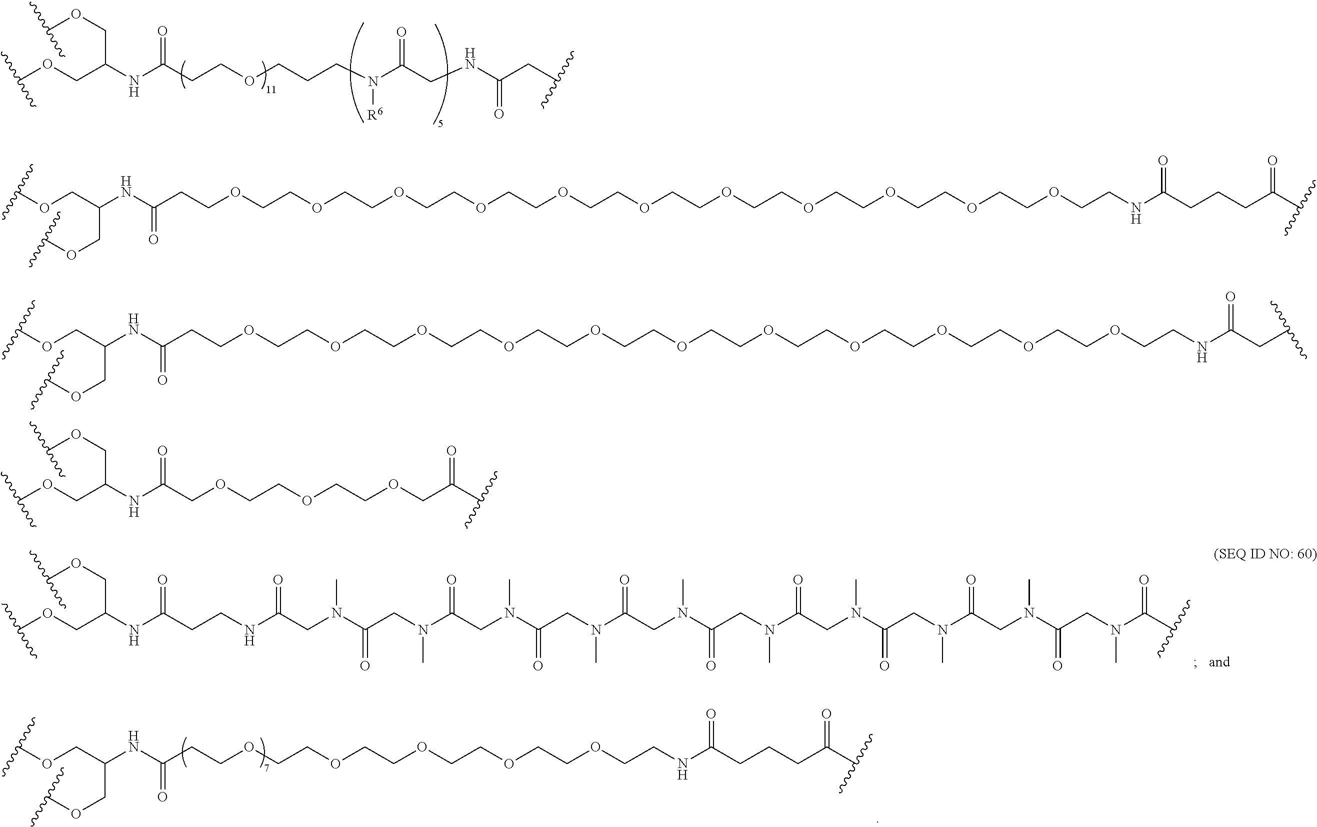

In certain embodiments, Linker^{C} is selected from:

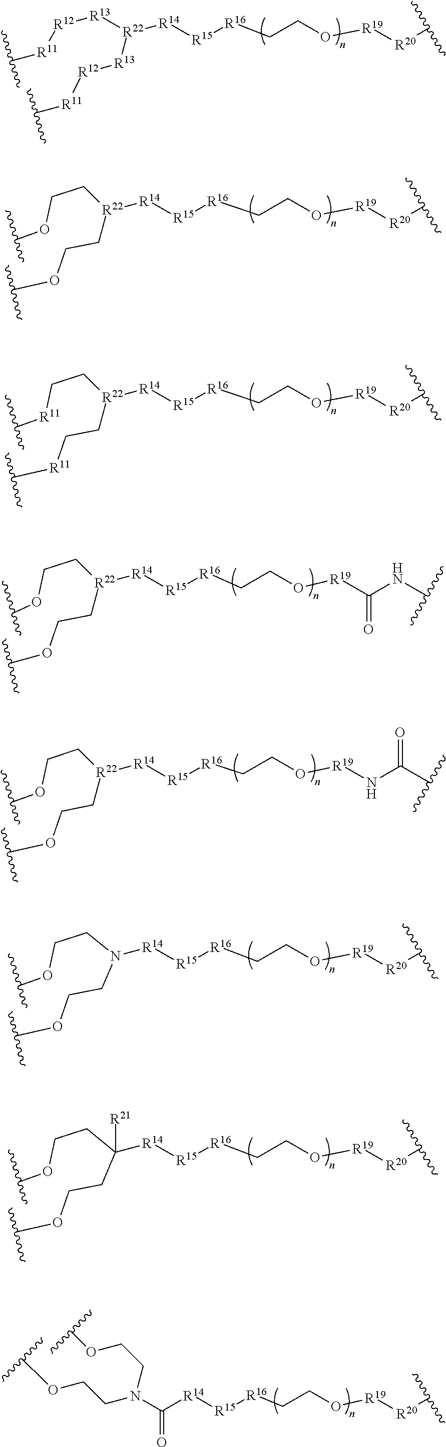

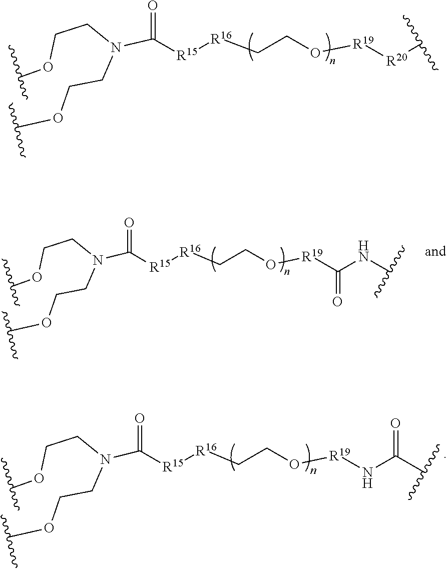

In certain embodiments, Linker^{A} or Linker^{B} is independently selected from

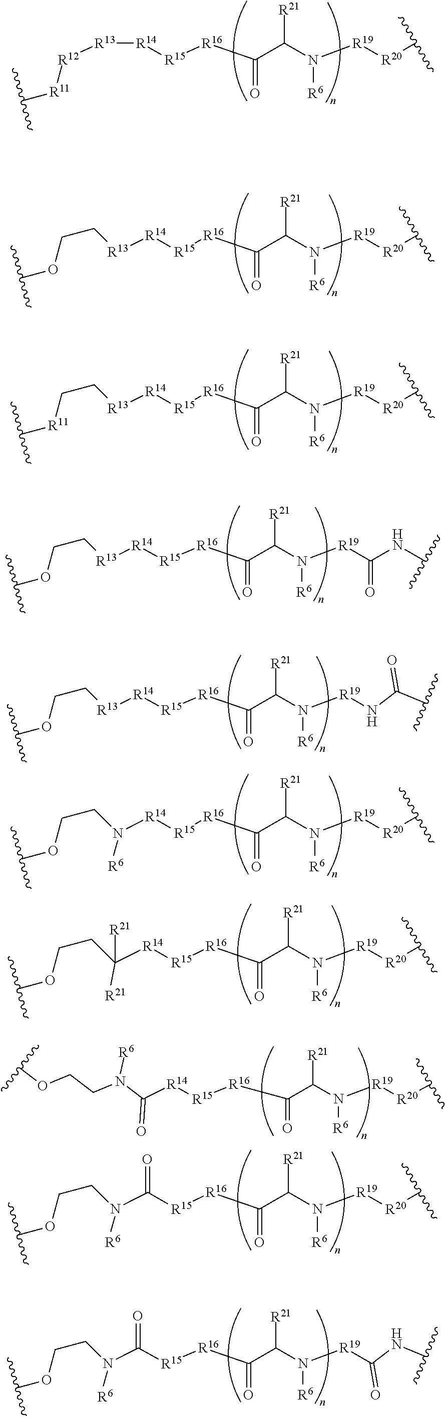

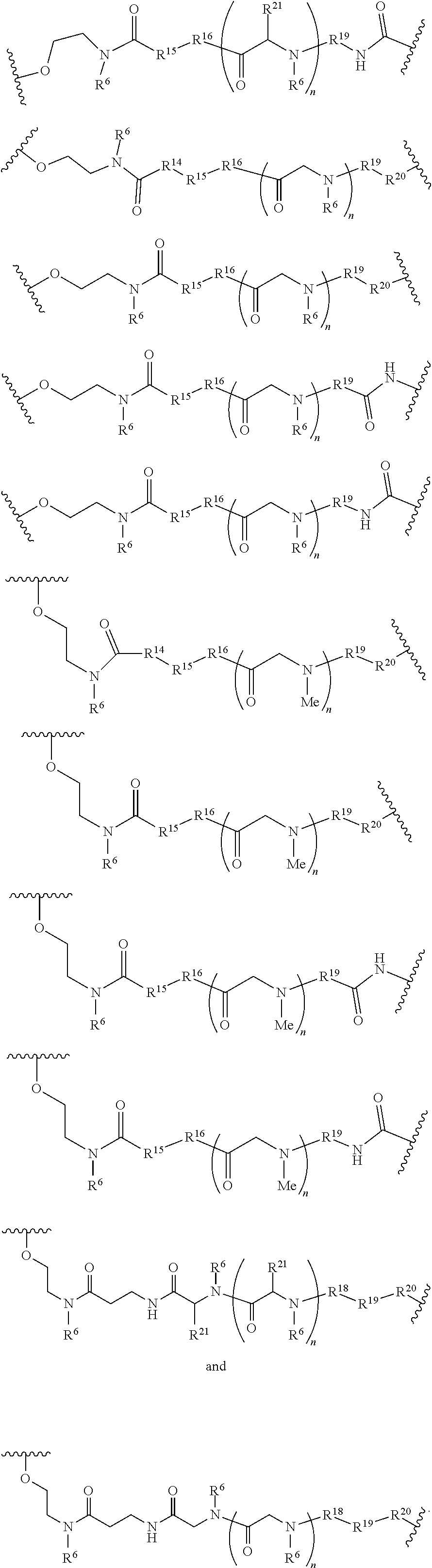

In certain embodiments, Linker^{A} or Linker^{B} is independently selected from

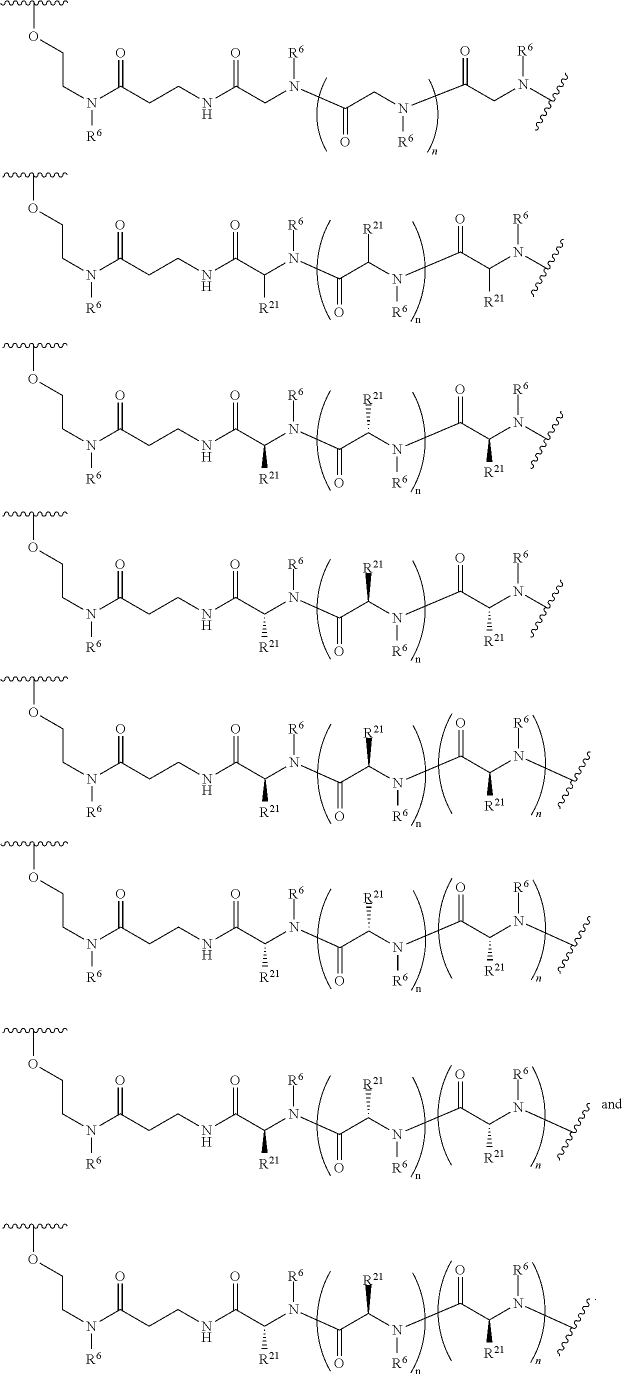

In certain embodiments, Linker^{A} or Linker^{B} is independently selected from

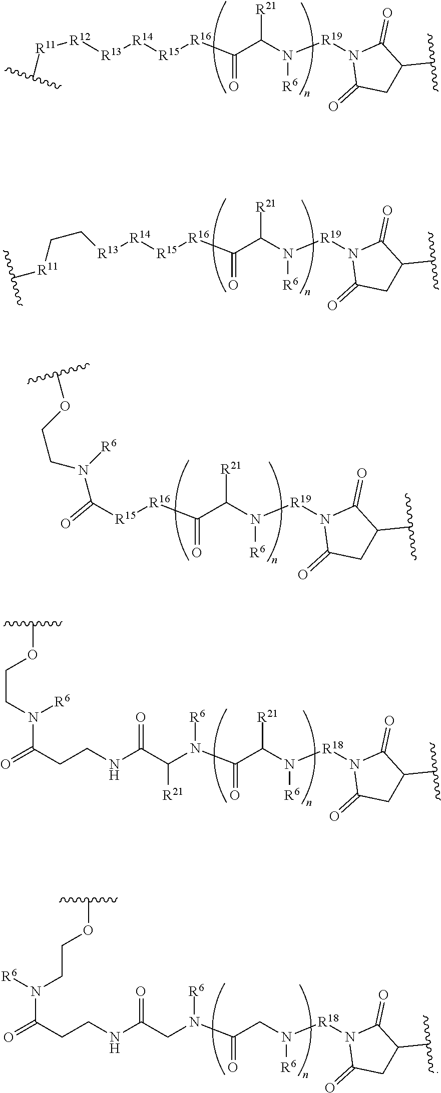

In certain embodiments, Linker^{A} or Linker^{B} is independently selected from

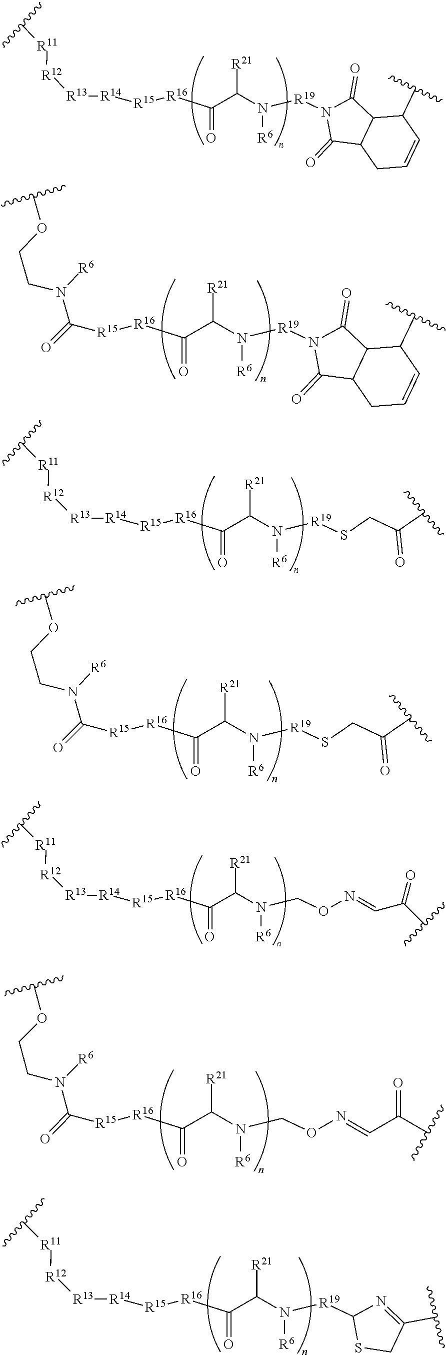

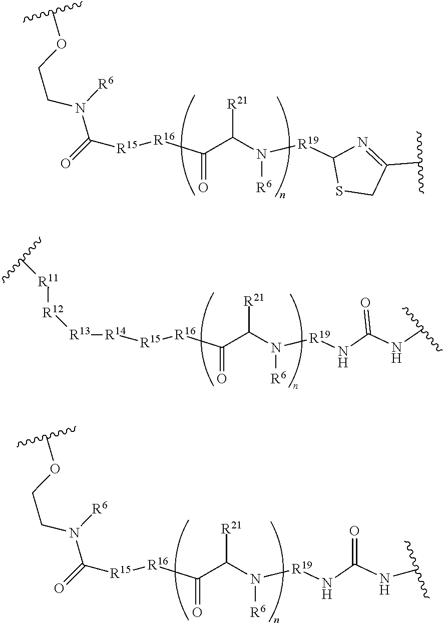

In certain embodiments, Linker^{A} or Linker^{B} is independently selected from

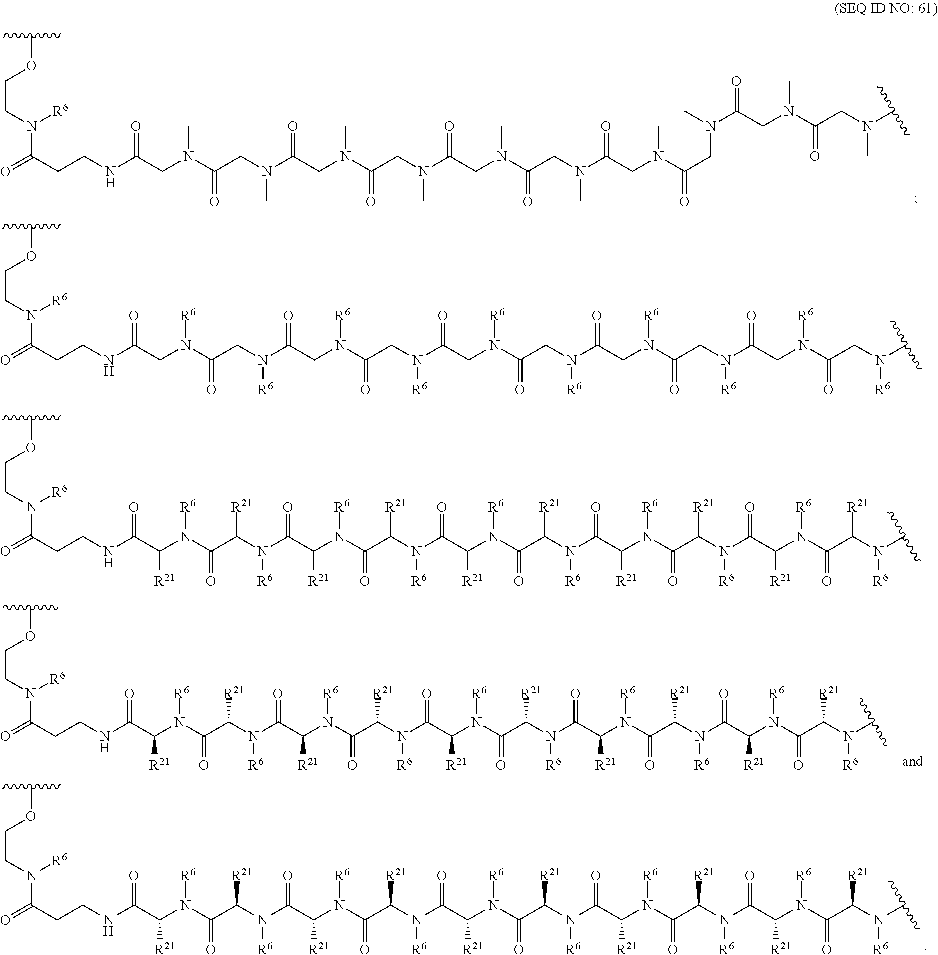

In certain embodiments, Linker^{A} or Linker^{B} is independently selected from:

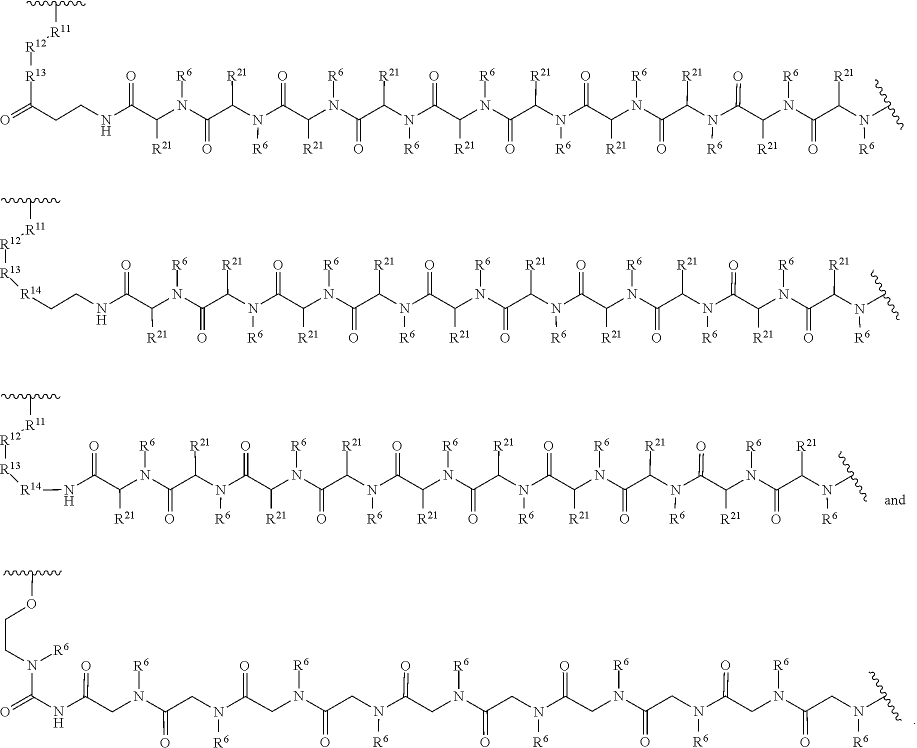

In certain embodiments, Linker^{A} or Linker^{B} is independently selected from

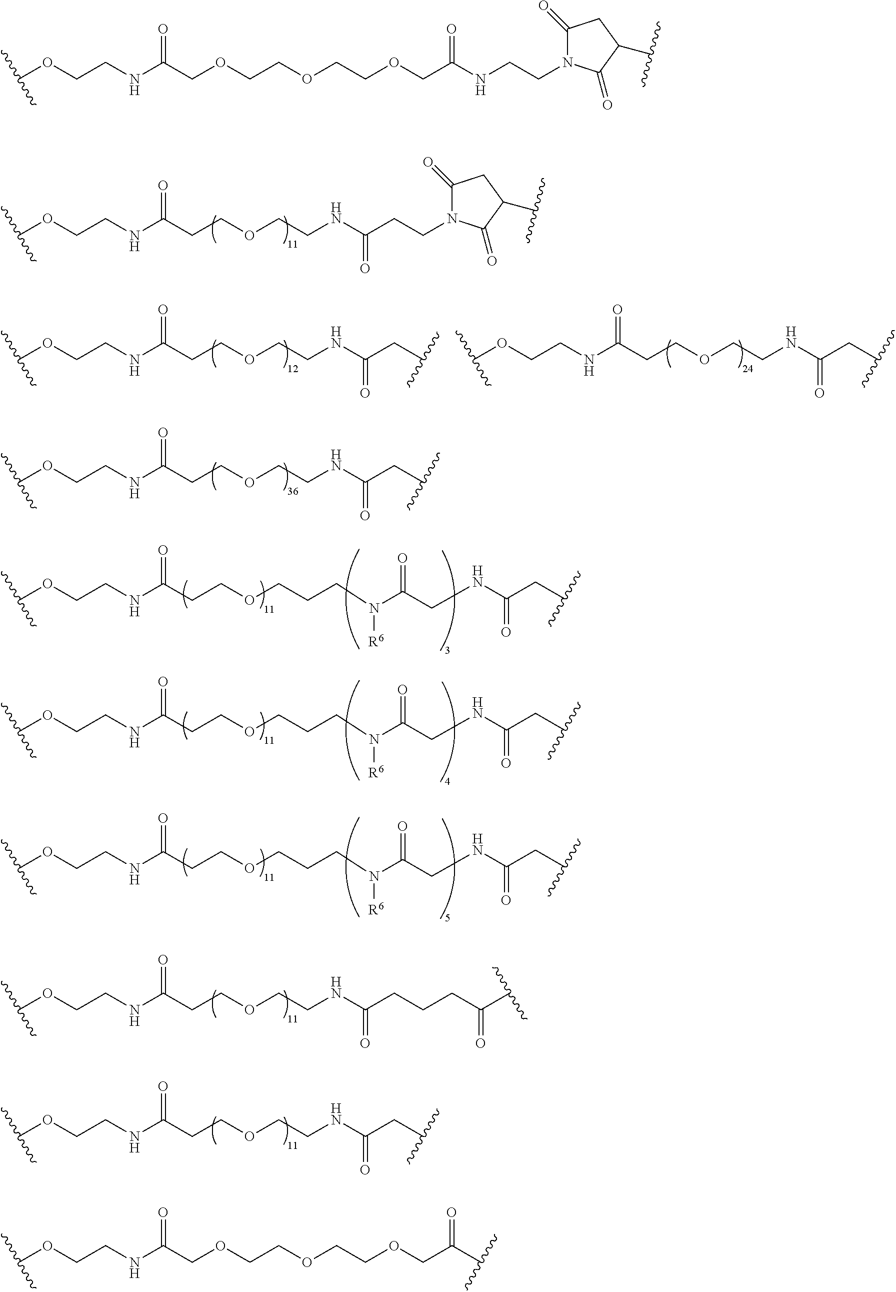

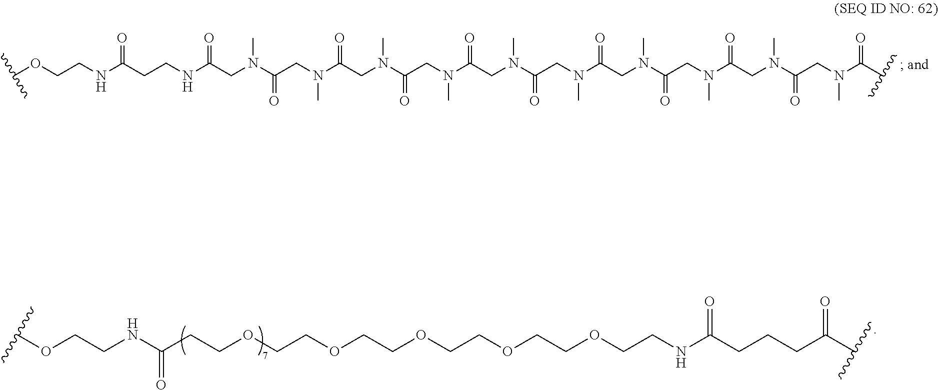

In certain embodiments, Linker^{A} or Linker^{B} is independently selected from:

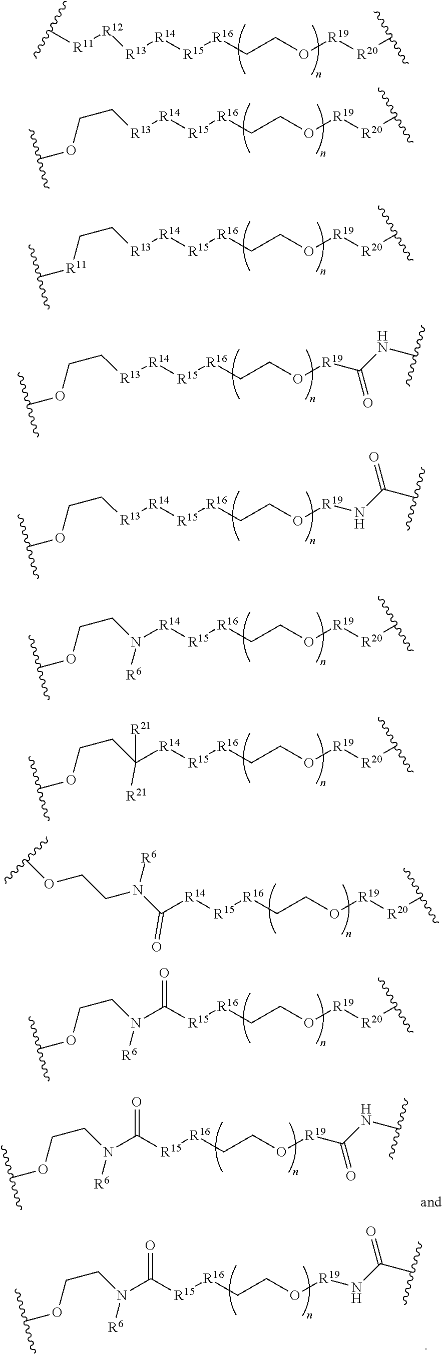

In certain embodiments, R¹¹, R¹², R¹³, R¹⁴, R¹⁵, R¹⁶, R¹⁷, R¹⁸, R¹⁹, and R²⁰ are bond. In certain embodiments, R¹², R¹³, R¹⁴, R¹⁵, R¹⁶, R¹⁷, R¹⁸, R¹⁹, and R²⁰ are bond. In certain embodiments, R¹³, R¹⁴, R¹⁵, R¹⁶, R¹⁷, R¹⁸, R¹⁹, and R²⁰ are bond. In certain embodiments, R¹⁴, R¹⁵, R¹⁶, R¹⁷, R¹⁸, R¹⁹, and R²⁰ are bond. In certain embodiments, R¹⁵, R¹⁶, R¹⁷, R¹⁸, R¹⁹, and R²⁰ are bond. In certain embodiments, R¹⁶, R¹⁷, R¹⁸, R¹⁹, and R²⁰ are bond. In certain embodiments, R¹⁷, R¹⁸, R¹⁹, and R²⁰ are bond. In certain embodiments, R¹⁸, R¹⁹, and R²⁰ are bond.

In certain embodiments, nine of R¹¹, R¹², R¹³, R¹⁴, R¹⁵, R¹⁶, R¹⁷, R¹⁸, R¹⁹, and R²⁰ are bond. In certain embodiments, eight of R¹¹, R¹², R¹³, R¹⁴, R¹⁵, R¹⁶, R¹⁷, R¹⁸, R¹⁹, and R²⁰ are bond. In certain embodiments, seven of R¹¹, R¹², R¹³, R¹⁴, R¹⁵, R¹⁶, R¹⁷, R¹⁸, R¹⁹, and R²⁰ are bond. In certain embodiments, six of R¹¹, R¹², R¹³, R¹⁴, R¹⁵, R¹⁶, R¹⁷, R¹⁸, R¹⁹, and R²⁰ are bond. In certain embodiments, five of R¹¹, R¹², R¹³, R¹⁴, R¹⁵, R¹⁶, R¹⁷, R¹⁸, R¹⁹, and R²⁰ are bond. In certain embodiments, four of R¹¹, R¹², R¹³, R¹⁴, R¹⁵, R¹⁶, R¹⁷, R¹⁸, R¹⁹, and R²⁰ are bond. In certain embodiments, three of R¹¹, R¹², R¹³, R¹⁴, R¹⁵, R¹⁶, R¹⁷, R¹⁸, R¹⁹, and R²⁰ are bond. In certain embodiments, two of R¹¹, R¹², R¹³, R¹⁴, R¹⁵, R¹⁶, R¹⁷, R¹⁸, R¹⁹, and R²⁰ are bond. In certain embodiments, one of R¹¹, R¹², R¹³, R¹⁴, R¹⁵, R¹⁶, R¹⁷, R¹⁸, R¹⁹, and R²⁰ is bond.

In certain embodiments, Linker^{A} is

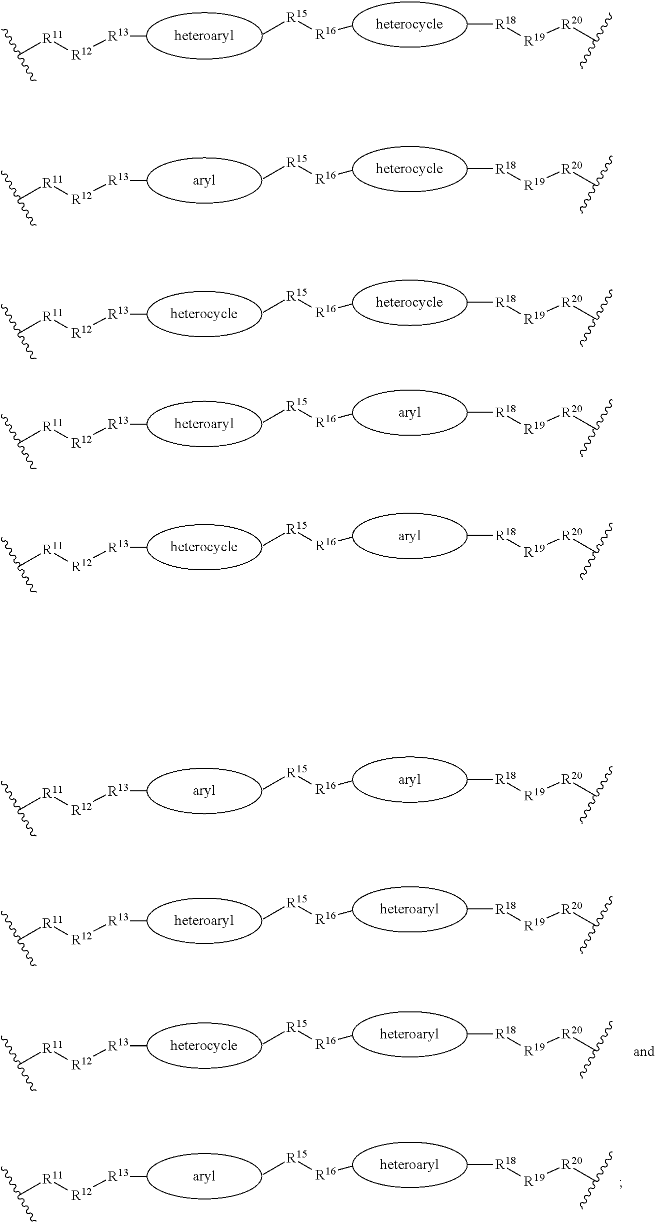

wherein each heteroaryl, heterocycle, and aryl can optionally be substituted with 1, 2, 3, or 4 of any combination of halogen, alkyl, haloalkyl, aryl, heteroaryl, heterocycle, or cycloalkyl.

In certain embodiments, R¹¹, R¹², R¹³, R¹⁵, R¹⁶, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)-alkenyl, alkynyl, haloalkyl, alkoxy, aryl, heterocycle, heteroaryl, and —CH₂CH₂—[O—(CH₂)₂]ₙ—O—.

In certain embodiments, Linker^{A} is selected from

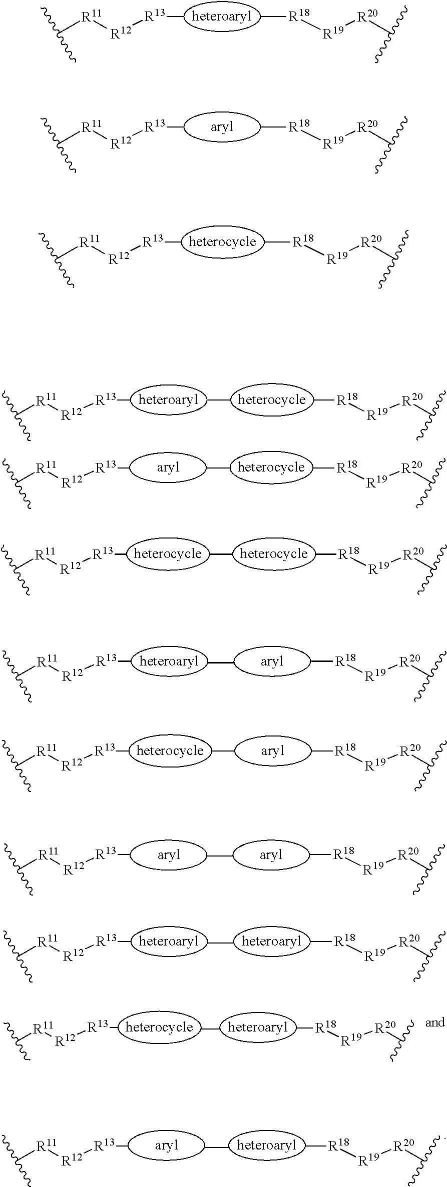

In certain embodiments, R¹¹, R¹², R¹³, R¹⁸, R¹⁹, and R²⁰ are independently selected from bond, alkyl, —C(O)—, —C(O)O—, —OC(O)—, —C(O)NR⁶—, —NWC(O)—, —O—, —S—, —NR—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, R¹¹ is selected from the group consisting of bond, CH₂, —O—, —C(O)NR⁶— and —C(O)O—. In certain embodiments, R²⁰ is selected from the group consisting of bond, CH₂, —O—, —C(O)NR⁶— and —C(O)O—.

In certain embodiments, Linker^{A} is selected from

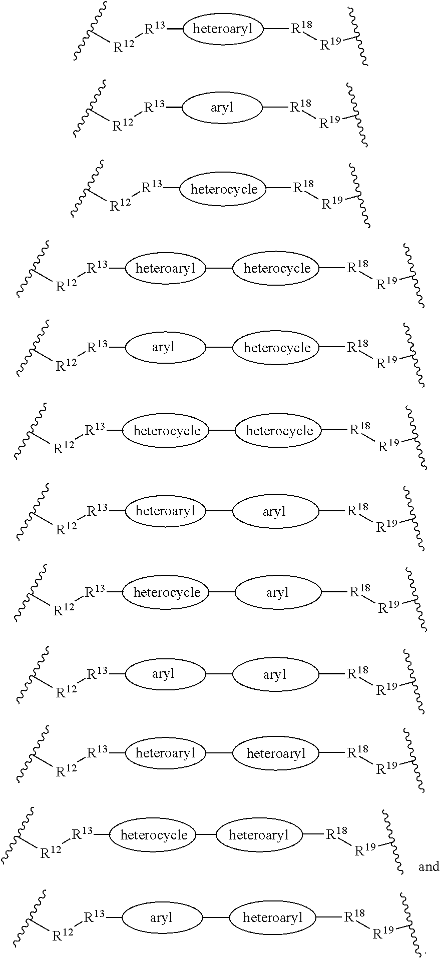

In certain embodiments, R¹² is selected from the group consisting of bond, CH₂, —O—, —C(O)NR⁶— and —C(O)O—. In certain embodiments, R¹⁹ is selected from the group consisting of bond, CH₂, —O—, —C(O)NR⁶— and —C(O)O—.

In certain embodiments, Linker^{A} is selected from

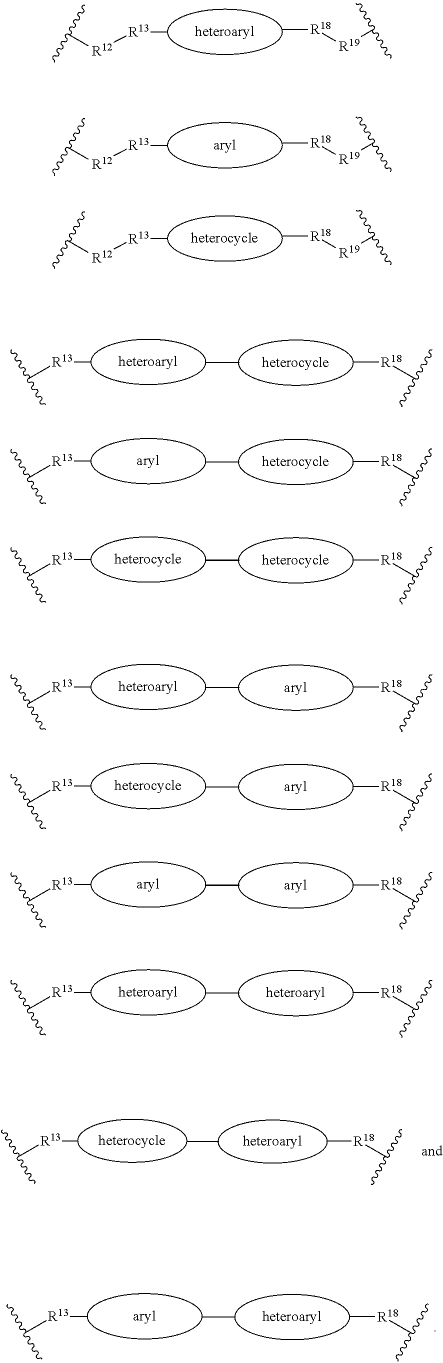

In certain embodiments, R¹³ is selected from the group consisting of bond, CH₂, —O—, —C(O)NR⁶— and —C(O)O—. In certain embodiments, R¹⁸ is selected from the group consisting of bond, CH₂, —O—, —C(O)NR⁶— and —C(O)O—.

In certain embodiments, aryl is phenyl.

In certain embodiments, heteroaryl is selected from

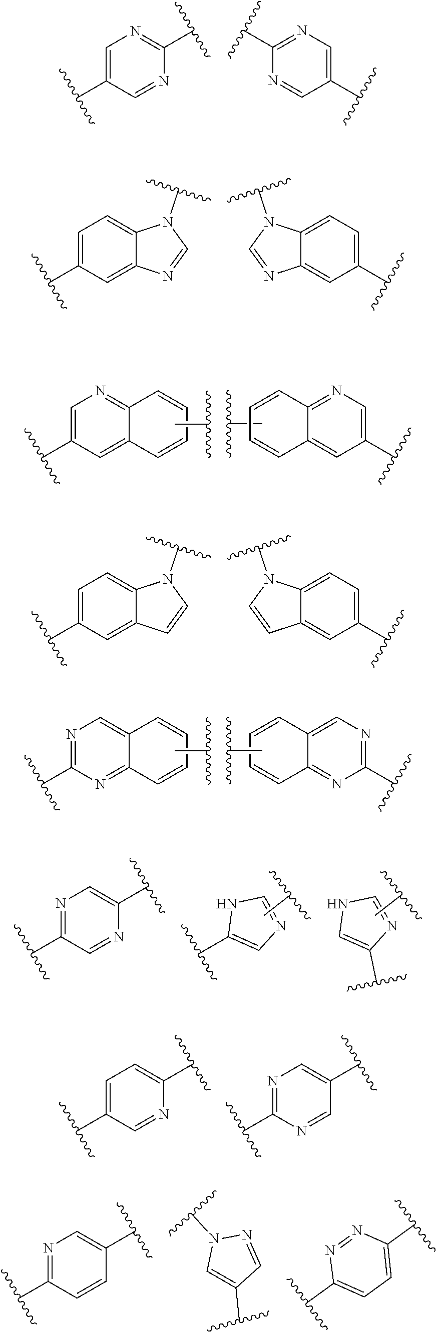

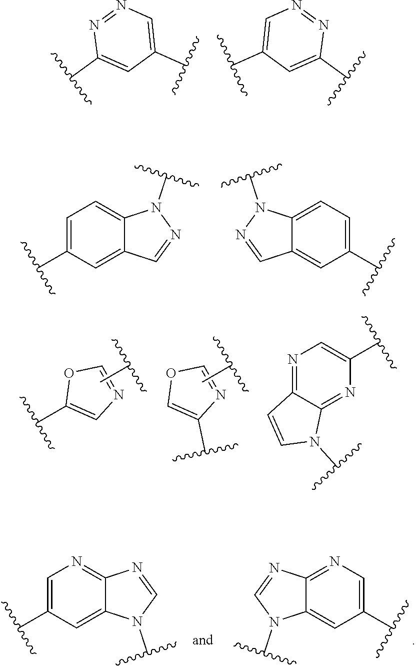

In certain embodiments, heteroaryl is selected from

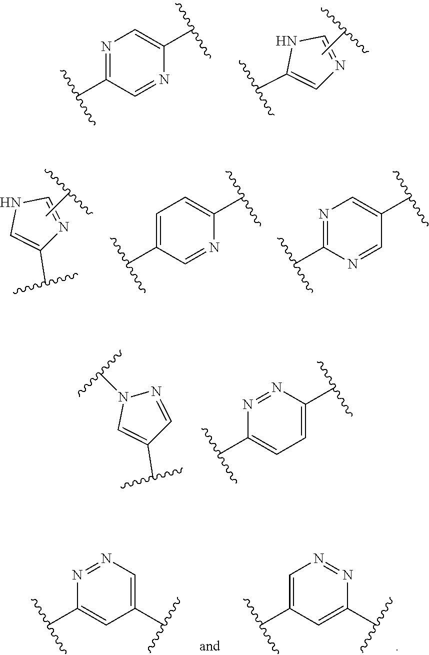

In certain embodiments, heterocycle is selected from

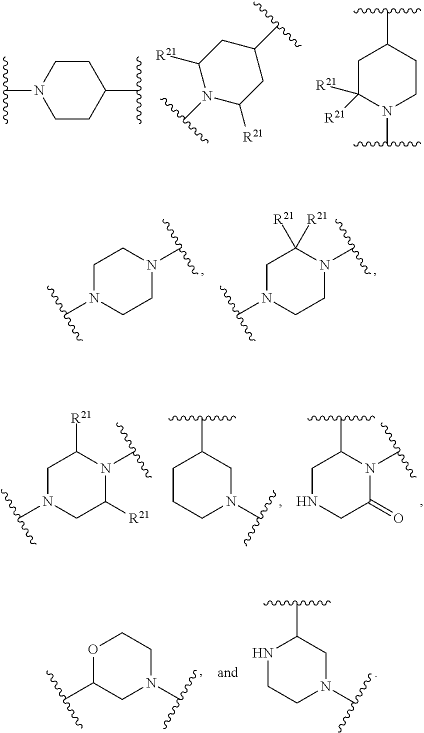

In certain embodiments, Linker^{A} is selected from

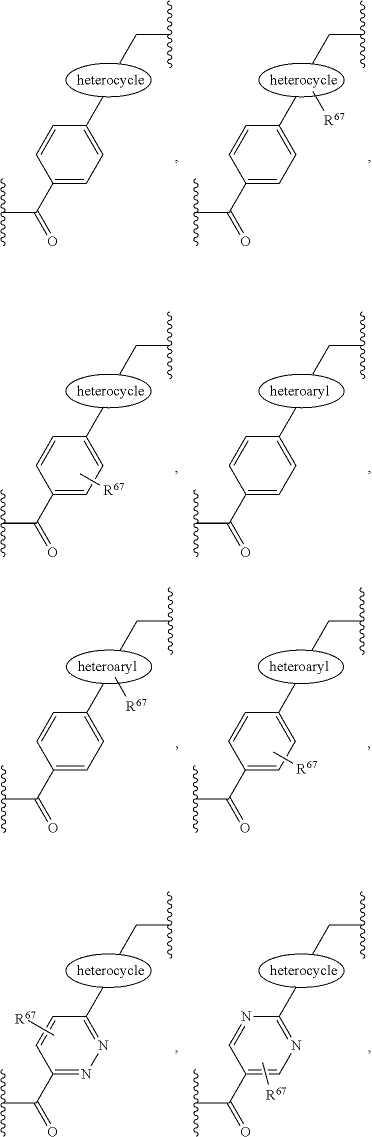

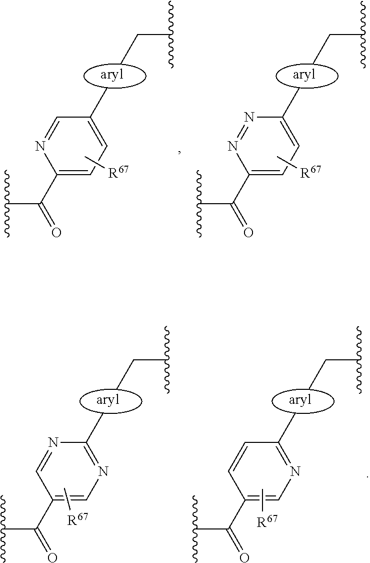

In certain embodiments, Linker^{A} is selected from

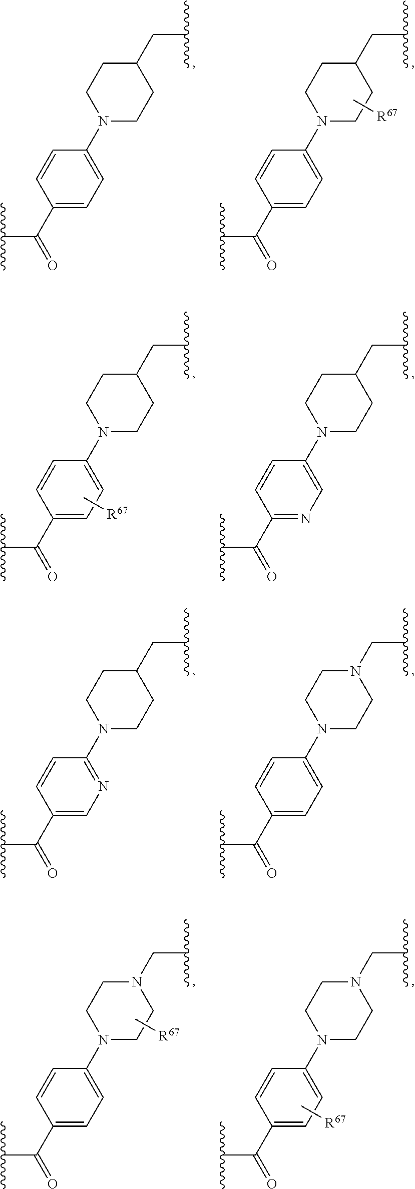

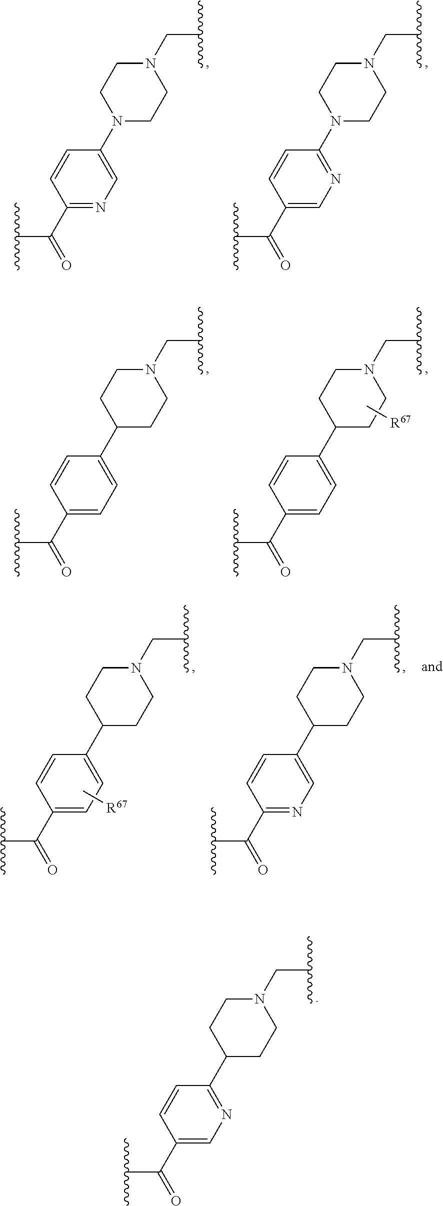

In certain embodiments Linker^{A} is selected from:

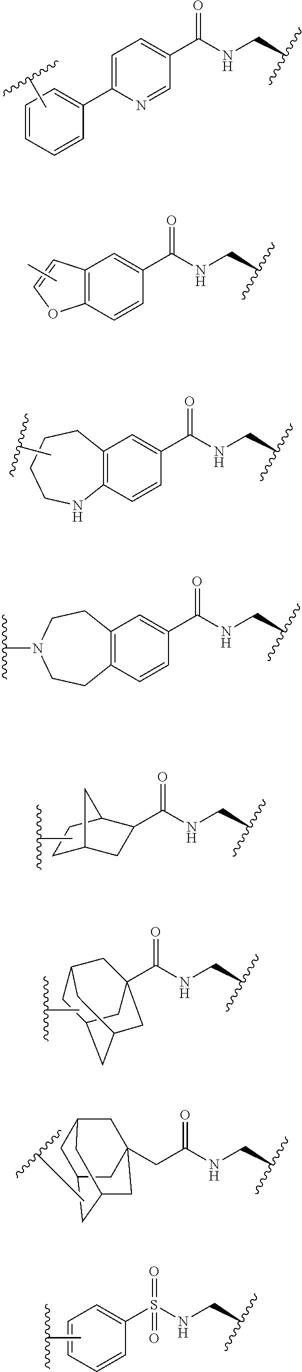

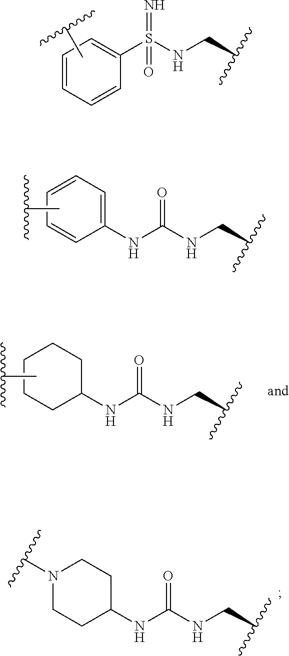

each of which is optionally substituted with 1, 2, 3, or 4 optional substituents as defined herein.

In certain embodiments, Linker^{B} is:

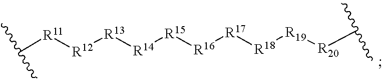

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, and —CH₂CH₂—[O—(CH₂)₂]ₙ—NR⁶—; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, and heterocycle.

In certain embodiments of Linker^{B}, R¹¹, R¹², R¹³, R¹⁴, R¹⁵, R¹⁶, and R¹⁷ are bond. In certain embodiments of Linker^{B}, five of R¹¹, R¹², R¹³, R¹⁴, R¹⁵, R¹⁶, R¹⁷, R¹⁸, and R¹⁹ are bond. In certain embodiments of Linker^{B}, four of R¹¹, R¹², R¹³, R¹⁴, R¹⁵, R¹⁶, R¹⁷, R¹⁸, and R¹⁹ are bond. In certain embodiments of Linker^{B}, three of R¹¹, R¹², R¹³, R¹⁴, R¹⁵, R¹⁶, R¹⁷, R¹⁸, and R¹⁹ are bond. In certain embodiments of Linker^{B}, R¹⁸, R¹⁹, and R²⁰ are independently selected from bond, alkyl, —C(O)—, —C(O)O—, —OC(O)—, —C(O)NR—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, R¹¹, R¹², R¹³, R¹⁴, R¹⁵, R¹⁶, R¹⁷, R¹⁸, R¹⁹, or R²⁰ is substituted with one R²¹ substituent.

In certain embodiments, R¹¹, R¹², R¹³, R¹⁴, R¹⁵, R¹⁶, R¹⁷, R¹⁸, R¹⁹, or R²⁰ is substituted with two R²¹ substituents, wherein the substituents are selected such that a stable compound results. For example, if R¹¹ is —(CR²¹R²¹)—, then the R²¹ groups are not independently hydroxyl and fluorine.

In certain embodiments, R¹¹, R¹², R¹³, R¹⁴, R¹⁵, R¹⁶, R¹⁷, R¹⁸, R¹⁹, or R²⁰ is substituted with three R²¹ substituents, wherein the substituents are selected such that a stable compound results.

In certain embodiments, R¹¹, R¹², R¹³, R¹⁴, R¹⁵, R¹⁶, R¹⁷, R¹⁸, R¹⁹, or R²⁰ is substituted with four R²¹ substituents, wherein the substituents are selected such that a stable compound results.

In certain embodiments, R²¹ is independently at each occurrence selected from the group consisting of hydrogen, alkyl, alkenyl, alkynyl, F, Cl, Br, I, hydroxyl, alkoxy, amino, —NR⁶R⁷, haloalkyl, aryl, heteroaryl, and heterocycle.

In certain embodiments, R²¹ is independently at each occurrence selected from the group consisting of hydrogen, alkyl, F, hydroxyl, alkoxy, amino, —NR⁶R⁷, haloalkyl, aryl, heteroaryl, and heterocycle.

In certain embodiments, R²¹ is independently at each occurrence selected from the group consisting of hydrogen, alkyl, F, hydroxyl, alkoxy, amino, —NR⁶R⁷, and haloalkyl.

### Additional Embodiments of Linker^{A}

In the embodiments of R¹¹, R¹², R¹³, R¹⁴, R¹⁵, R¹⁶, R¹⁷, R¹⁸, R¹⁹, and R²⁰ provided herein the structure can be included in the Linker as read from left to right or alternatively as read from right to left unless excluded by context. For example, when R¹⁵ is

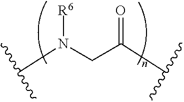

### and Linker^{A} is

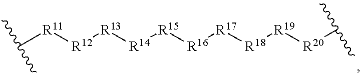

### Linker^{A} can be

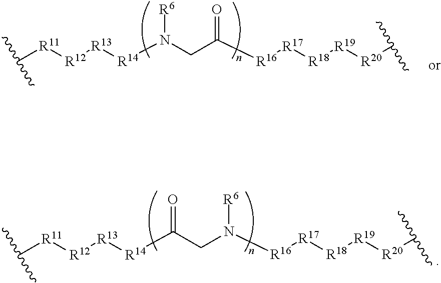

A1. In certain embodiments of Linker^{A}, R¹¹ is bond.
A2. In certain embodiments of Linker^{A}, R¹¹ is alkyl.
A3. In certain embodiments of Linker^{A}, R¹¹ is —C(O)—.
A4. In certain embodiments of Linker^{A}, R¹¹ is —C(O)O—.
A5. In certain embodiments of Linker^{A}, R¹¹ is —C(O)NR⁶—.
A6. In certain embodiments of Linker^{A}, R¹¹ is —NR⁶C(O)—.
A7. In certain embodiments of Linker^{A}, R¹¹ is —NR⁶—.
A8. In certain embodiments of Linker^{A}, R¹¹ is —O—.
A9. In certain embodiments of Linker^{A}, R¹¹ is —C(R²¹R²¹)—.
A10. The Linker^{A} of embodiment A9, wherein both R²¹ groups are hydrogen.
A1 l. In certain embodiments of Linker^{A}, R¹¹ is —CH₂CH₂—[O—(CH₂)₂]ₙ—O—.
A12. In certain embodiments of Linker^{A}, R¹¹ is aryl.
A13. The Linker^{A} of embodiment A12, wherein R¹¹ is phenyl.
A14. The Linker^{A} of embodiment A12, wherein R¹¹ is naphthyl.
A15. In certain embodiments of Linker^{A}, R¹¹ is heterocycle.
A16. The Linker^{A} of embodiment A15, wherein R¹¹ is piperidinyl.
A17. The Linker^{A} of embodiment A16, wherein R¹¹ is

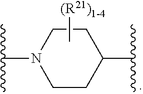

A18. The Linker^{A} of embodiment A16, wherein R¹¹ is

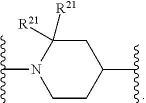

A19. The Linker^{A} of embodiment A15, wherein R¹¹ is piperizinyl.
A20. The Linker^{A} of embodiment A19, wherein R¹¹ is

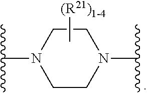

A21. The Linker^{A} of embodiment A15, wherein R¹¹ is pyrrolidinyl.
A22. The Linker^{A} of embodiment A21, wherein R¹¹ is

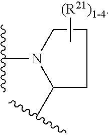

A23. The Linker^{A} of embodiment A21, wherein R¹¹ is

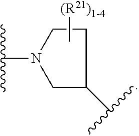

A24. In certain embodiments of Linker^{A}, R¹¹ is heteroaryl.
A25. The Linker^{A} of embodiment A24, wherein R¹¹ is pyridinyl.
A26. The Linker^{A} of embodiment A25, wherein R¹¹ is

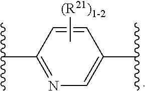

A27. The Linker^{A} of embodiment A25, wherein R¹¹ is

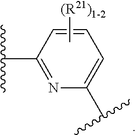

A28. The Linker^{A} of embodiment A25, wherein R¹¹ is

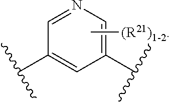

A29. The Linker^{A} of embodiment A24, wherein R¹¹ is pyrimidinyl.
A30. The Linker^{A} of embodiment A29, wherein R¹¹ is

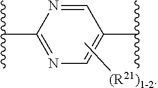

A31. The Linker^{A} of embodiment A29, wherein R¹¹ is

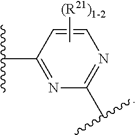

A32. The Linker^{A} of embodiment A24, wherein R¹¹ is pyrazinyl.
A33. The Linker^{A} of embodiment A32, wherein R¹¹ is

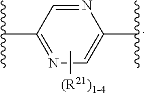

A34. The Linker^{A} of embodiment A32, wherein R¹¹ is

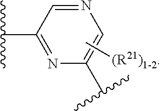

A35. The Linker^{A} of embodiment A24, wherein R¹¹ is pyridizinyl.
A36. The Linker^{A} of embodiment A35, wherein R¹¹ is

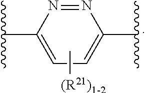

A37. The Linker^{A} of embodiment A35, wherein R¹¹ is

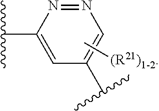

A38. In certain embodiments of Linker^{A}, R¹¹ is alkynyl.
A39. In certain embodiments of Linker^{A}, R¹¹ is alkoxy.
A40. The Linker^{A} of any one of embodiments A1-A39, wherein R¹² is bond.
A41. The Linker^{A} of any one of embodiments A1-A39, wherein R¹² is alkyl.
A42. The Linker^{A} of any one of embodiments A1-A39, wherein R¹² is —C(O)—.
A43. The Linker^{A} of any one of embodiments A1-A39, wherein R¹² is —C(O)O—.
A44. The Linker^{A} of any one of embodiments A1-A39, wherein R¹² is —C(O)NR⁶—.
A45. The Linker^{A} of any one of embodiments A1-A39, wherein R¹² is —NR⁶C(O)—.
A46. The Linker^{A} of any one of embodiments A1-A39, wherein R¹² is —C(R²¹R²¹)—.
A47. The Linker^{A} of embodiment A46, wherein both R²¹ groups are hydrogen.
A48. The Linker^{A} of any one of embodiments A1-A39, wherein R¹² is —CH₂CH₂—[O—(CH₂)₂]ₙ—O—.
A49. The Linker^{A} of any one of embodiments A1-A39, wherein R¹² is aryl.
A50. The Linker^{A} of embodiment A39, wherein R¹² is phenyl.
A51. The Linker^{A} of embodiment A39, wherein R¹² is naphthyl.
A52. The Linker^{A} of any one of embodiments A1-A39, wherein R¹² is heterocycle.
A53. The Linker^{A} of embodiment A52, wherein R¹² is piperidinyl.
A54. The Linker^{A} of embodiment A53, wherein R¹² is

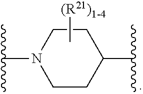

A55. The Linker^{A} of embodiment A53, wherein R¹² is

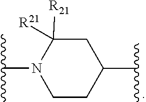

A56. The Linker^{A} of embodiment A52, wherein R¹² is piperizinyl.
A57. The Linker^{A} of embodiment A56, wherein R¹² is

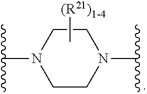

A58. The Linker^{A} of embodiment A52, wherein R¹² is pyrrolidinyl.
A59. The Linker^{A} of embodiment A58, wherein R¹² is

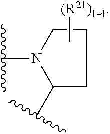

A60. The Linker^{A} of embodiment A59, wherein R¹² is

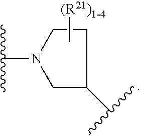

A61. The Linker^{A} of any one of embodiments A1-A39, wherein R¹² is heteroaryl.
A62. The Linker^{A} of embodiment A61, wherein R¹² is pyridinyl.
A63. The Linker^{A} of embodiment A62, wherein R¹² is N

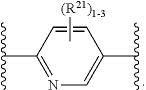

A64. The Linker^{A} of embodiment A62, wherein R¹² is

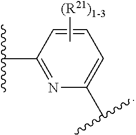

A65. The Linker^{A} of embodiment A62, wherein R¹² is

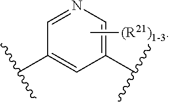

A66. The Linker^{A} of embodiment A61, wherein R¹² is pyrimidinyl.
A67. The Linker^{A} of embodiment A66, wherein R¹² is (R²¹)

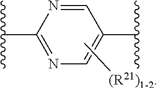

A68. The Linker^{A} of embodiment A66, wherein R¹² is

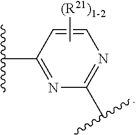

A69. The Linker^{A} of embodiment A61, wherein R¹² is pyrazinyl.
A70. The Linker^{A} of embodiment A69, wherein R¹² is (R²)

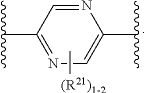

A71. The Linker^{A} of embodiment A68, wherein R¹² is

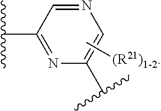

A72. The Linker^{A} of embodiment A61, wherein R¹² is pyridizinyl.
A73. The Linker^{A} of embodiment A72, wherein R¹² is (R²)₁-2

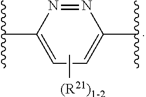

A74. The Linker^{A} of embodiment A72, wherein R¹² is

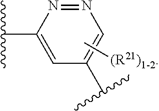

A75. The Linker^{A} of any one of embodiments A1-A39, wherein R¹² is alkynyl.
A76. The Linker^{A} of any one of embodiments A1-A39, wherein R¹² is alkoxy.
A77. The Linker^{A} of any one of embodiments A1-A76, wherein R¹³ is bond.
A78. The Linker^{A} of any one of embodiments A1-A76, wherein R¹³ is alkyl.
A79. The Linker^{A} of any one of embodiments A1-A76, wherein R¹³ is —C(O)—.
A80. The Linker^{A} of any one of embodiments A1-A76, wherein R¹³ is —C(O)O—.
A81. The Linker^{A} of any one of embodiments A1-A76, wherein R¹³ is —C(O)NR⁶—.
A82. The Linker^{A} of any one of embodiments A1-A76, wherein R¹³ is —NR⁶C(O)—.
A83. The Linker^{A} of any one of embodiments A1-A76, wherein R¹³ is —NR⁶—.
A84. The Linker^{A} of any one of embodiments A1-A76, wherein R¹³ is —O—.
A85. The Linker^{A} of any one of embodiments A1-A76, wherein R¹³ is —C(R²¹R²¹)—.
A86. The Linker^{A} of embodiment A85, wherein both R²¹ groups are hydrogen.
A87. The Linker^{A} of any one of embodiments A1-A76, wherein R¹³ is —CH₂CH₂—[O—(CH₂)₂]ₙ—O—.
A88. The Linker^{A} of any one of embodiments A1-A76, wherein R¹³ is aryl.
A89. The Linker^{A} of embodiment A88, wherein R¹³ is phenyl.
A90. The Linker^{A} of embodiment A88, wherein R¹³ is naphthyl.
A91. The Linker^{A} of any one of embodiments A1-A76, wherein R¹³ is heterocycle.
A92. The Linker^{A} of embodiment A91, wherein R¹³ is piperidinyl.
A93. The Linker^{A} of embodiment A92, wherein R¹³ is

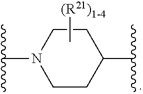

A94. The Linker^{A} of embodiment A92, wherein R¹³ is

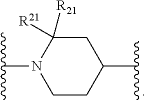

A95. The Linker^{A} of embodiment A91, wherein R¹³ is piperizinyl.
A96. The Linker^{A} of embodiment A95, wherein R¹³ is

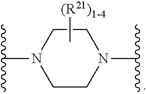

A97. The Linker^{A} of embodiment A91, wherein R¹³ is pyrrolidinyl.
A98. The Linker^{A} of embodiment A97, wherein R¹³ is

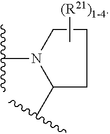

A99. The Linker^{A} of embodiment A97, wherein R¹³ is

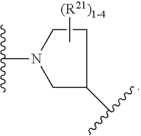

A100. The Linker^{A} of any one of embodiments A1-A76, wherein R¹³ is heteroaryl.
A101. The Linker^{A} of embodiment A100, wherein R¹³ is pyridinyl.
A102. The Linker^{A} of embodiment A101, wherein R¹³ is

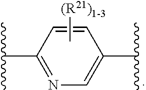

A103. The Linker^{A} of embodiment A101, wherein R¹³ is

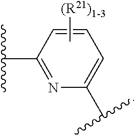

A104. The Linker^{A} of embodiment A101, wherein R¹³ is

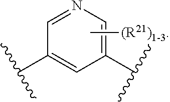

A105. The Linker^{A} of embodiment A100, wherein R¹³ is pyrimidinyl.
A106. The Linker^{A} of embodiment A105, wherein R¹³ is

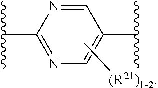

A107. The Linker^{A} of embodiment A105, wherein R¹³ is

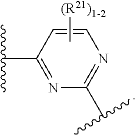

A108. The Linker^{A} of embodiment A100, wherein R¹³ is pyrazinyl.
A109. The Linker^{A} of embodiment A108, wherein R¹³ is

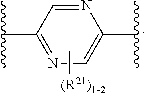

A110. The Linker^{A} of embodiment A108, wherein R¹³ is

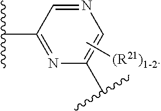

A111. The Linker^{A} of embodiment A100, wherein R¹³ is pyridizinyl.
A112. The Linker^{A} of embodiment A111, wherein R¹³ is

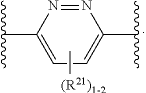

A113. The Linker^{A} of embodiment A111, wherein R¹³ is

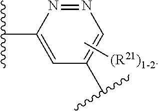

A114. The Linker^{A} of any one of embodiments A1-A76, wherein R¹³ is alkynyl.
A115. The Linker^{A} of any one of embodiments A1-A76, wherein R¹³ is alkoxy.
A116. The Linker^{A} of any one of embodiments A1-A115, wherein R¹⁴ is bond.
A117. The Linker^{A} of any one of embodiments A1-A115, wherein R¹⁴ is alkyl.
A118. The Linker^{A} of any one of embodiments A1-A115, wherein R¹⁴ is —C(O)—.
A119. The Linker^{A} of any one of embodiments A1-A115, wherein R¹⁴ is —C(O)O—.
A120. The Linker^{A} of any one of embodiments A1-A115, wherein R¹⁴ is —C(O)NR⁶—.
A121. The Linker^{A} of any one of embodiments A1-A115, wherein R¹⁴ is —NR⁶C(O)—.
A122. The Linker^{A} of any one of embodiments A1-A115, wherein R¹⁴ is —S(O)—.
A123. The Linker^{A} of any one of embodiments A1-A115, wherein R¹⁴ is —S(O)₂—.
A124. The Linker^{A} of any one of embodiments A1-A115, wherein R¹⁴ is —C(R²¹R²¹)—.
A125. The Linker^{A} of embodiment A124, wherein both R²¹ groups are hydrogen.
A126. The Linker^{A} of any one of embodiments A1-A115, wherein R¹⁴ is —CH₂CH₂—[O—(CH₂)₂]ₙ—O—.
A127. The Linker^{A} of any one of embodiments A1-A115, wherein R¹⁴ is aryl.
A128. The Linker^{A} of embodiment A127, wherein R¹⁴ is phenyl.
A129. The Linker^{A} of embodiment A127, wherein R¹⁴ is naphthyl.
A130. The Linker^{A} of any one of embodiments A1-A115, wherein R¹⁴ is heterocycle.
A131. The Linker^{A} of embodiment A130, wherein R¹⁴ is piperidinyl.
A132. The Linker^{A} of embodiment A131, wherein R¹⁴ is

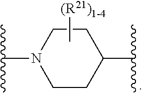

A133. The Linker^{A} of embodiment A131, wherein R¹⁴ is

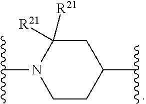

A134. The Linker^{A} of embodiment A130, wherein R¹⁴ is piperizinyl.
A135. The Linker^{A} of embodiment A134, wherein R¹⁴ is

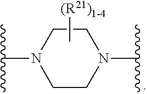

A136. The Linker^{A} of embodiment A130, wherein R¹⁴ is pyrrolidinyl.
A137. The Linker^{A} of embodiment A136, wherein R¹⁴ is

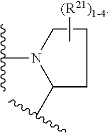

A138. The Linker^{A} of embodiment A136, wherein R¹⁴ is

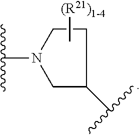

A139. The Linker^{A} of any one of embodiments A1-A115, wherein R¹⁴ is heteroaryl.
A140. The Linker^{A} of embodiment A139, wherein R¹⁴ is pyridinyl.
A141. The Linker of embodiment A140, wherein R¹⁴ is

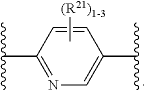

A142. The Linker^{A} of embodiment A140, wherein R¹⁴ is

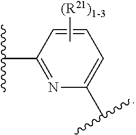

A143. The Linker^{A} of embodiment A140, wherein R¹⁴ is

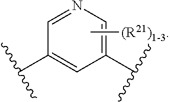

A144. The Linker^{A} of embodiment A139, wherein R¹⁴ is pyrimidinyl.
A145. The Linker^{A} of embodiment A144, wherein R¹⁴ is

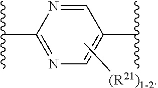

A146. The Linker^{A} of embodiment A144, wherein R¹⁴ is

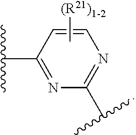

A147. The Linker^{A} of embodiment A139, wherein R¹⁴ is pyrazinyl.
A148. The Linker^{A} of embodiment A147, wherein R¹⁴ is

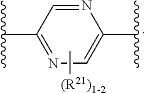

A149. The Linker^{A} of embodiment A147, wherein R¹⁴ is

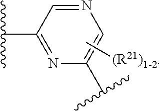

A150. The Linker^{A} of embodiment A139, wherein R¹⁴ is pyridizinyl.
A151. The Linker^{A} of embodiment A150, wherein R¹⁴ is

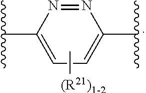

A152. The Linker^{A} of embodiment A150, wherein R¹⁴ is

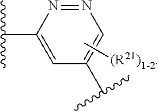

A153. The Linker^{A} of any one of embodiments A1-A115, wherein R¹⁴ is alkynyl.
A154. The Linker^{A} of any one of embodiments A1-A115, wherein R¹⁴ is alkoxy.
A155. The Linker^{A} of any one of embodiments A1-A154, wherein R¹⁵ is bond.
A156. The Linker^{A} of any one of embodiments A1-A154, wherein R¹⁵ is alkyl.
A157. The Linker^{A} of any one of embodiments A1-A154, wherein R¹⁵ is —C(O)—.
A158. The Linker^{A} of any one of embodiments A1-A154, wherein R¹⁵ is —C(O)O—.
A159. The Linker^{A} of any one of embodiments A1-A154, wherein R¹⁵ is —C(O)NR⁶—.
A160. The Linker^{A} of any one of embodiments A1-A154, wherein R¹⁵ is —NR⁶C(O)—.
A161. The Linker^{A} of any one of embodiments A1-A154, wherein R¹⁵ is —NR⁶—.
A162. The Linker^{A} of any one of embodiments A1-A154, wherein R¹⁵ is —O—.
A163. The Linker^{A} of any one of embodiments A1-A154, wherein R¹⁵ is —C(R²¹R²¹)—.
A164. The Linker^{A} of embodiment A163, wherein both R²¹ groups are hydrogen.
A165. The Linker^{A} of any one of embodiments A1-A154, wherein R¹⁵ is —CH₂CH₂—[O—(CH₂)₂]ₙ—O—.
A166. The Linker^{A} of any one of embodiments A1-A154, wherein R¹⁵ is aryl.
A167. The Linker^{A} of embodiment A166, wherein R¹⁵ is phenyl.
A168. The Linker^{A} of embodiment A166, wherein R¹⁵ is naphthyl.
A169. The Linker^{A} of any one of embodiments A1-A154, wherein R¹⁵ is heterocycle.
A170. The Linker^{A} of embodiment A169, wherein R¹⁵ is piperidinyl.
A171. The Linker^{A} of embodiment A170, wherein R¹⁵ is

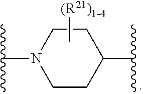

A172. The Linker^{A} of embodiment A170, wherein R¹⁵ is

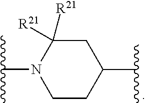

A173. The Linker^{A} of embodiment A169, wherein R¹⁵ is piperizinyl.
A174. The Linker^{A} of embodiment A173, wherein R¹⁵ is

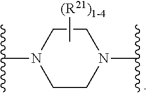

A175. The Linker^{A} of embodiment A169, wherein R¹⁵ is pyrrolidinyl.
A176. The Linker^{A} of embodiment A175, wherein R¹⁵ is

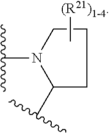

A177. The Linker^{A} of embodiment A175, wherein R¹⁵ is

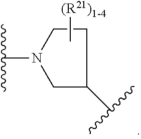

A178. The Linker^{A} of any one of embodiments A1-A154, wherein R¹⁵ is heteroaryl.
A179. The Linker^{A} of embodiment A178, wherein R¹⁵ is pyridinyl.
A180. The Linkerof embodiment A179, wherein R⁵ is

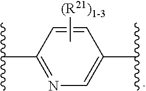

A181. The Linker^{A} of embodiment A179, wherein R¹⁵ is

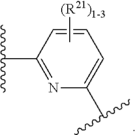

A182. The Linker^{A} of embodiment A179, wherein R¹⁵ is

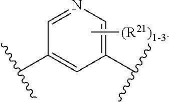

A183. The Linker^{A} of embodiment A178, wherein R¹⁵ is pyrimidinyl.
A184. The Linker^{A} of embodiment A183, wherein R¹⁵ is

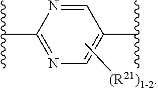

A185. The Linker^{A} of embodiment A183, wherein R¹⁵ is

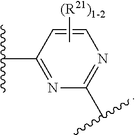

A186. The Linker^{A} of embodiment A178, wherein R¹⁵ is pyrazinyl.
A187. The Linker^{A} of embodiment A186, wherein R¹⁵ is

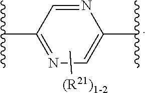

A188. The Linker^{A} of embodiment A186, wherein R¹⁵ is

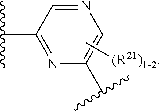

A189. The Linker^{A} of embodiment A178, wherein R¹⁵ is pyridizinyl.
A190. The Linker^{A} of embodiment A189, wherein R¹⁵ is

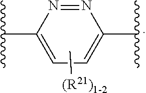

A191. The Linker^{A} of embodiment A189, wherein R¹⁵

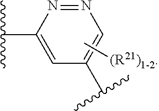

A192. The Linker^{A} of any one of embodiments A1-A154, wherein R¹⁵ is alkynyl.
A193. The Linker^{A} of any one of embodiments A1-A154, wherein R¹⁵ is alkoxy.
A194. The Linker^{A} of any one of embodiments A1-A193, wherein R¹⁶ is bond.
A195. The Linker^{A} of any one of embodiments A1-A193, wherein R¹⁶ is alkyl.
A196. The Linker^{A} of any one of embodiments A1-A193, wherein R¹⁶ is —C(O)—.
A197. The Linker^{A} of any one of embodiments A1-A193, wherein R¹⁶ is —C(O)O—.
A198. The Linker^{A} of any one of embodiments A1-A193, wherein R¹⁶ is —C(O)NR⁶—.
A199. The Linker^{A} of any one of embodiments A1-A193, wherein R¹⁶ is —NR⁶C(O)—.
A200. The Linker^{A} of any one of embodiments A1-A193, wherein R¹⁶ is —S(O)—.
A201. The Linker^{A} of any one of embodiments A1-A193, wherein R¹⁶ is —S(O)₂—.
A202. The Linker^{A} of any one of embodiments A1-A193, wherein R¹⁶ is —C(R²¹R²¹)—.
A203. The Linker^{A} of embodiment A202, wherein both R²¹ groups are hydrogen.
A204. The Linker^{A} of any one of embodiments A1-A193, wherein R¹⁶ is —CH₂CH₂—[O—(CH₂)₂]ₙ—O—.
A205. The Linker^{A} of any one of embodiments A1-A193, wherein R¹⁶ is aryl.
A206. The Linker^{A} of embodiment A205, wherein R¹⁶ is phenyl.
A207. The Linker^{A} of embodiment A205, wherein R¹⁶ is naphthyl.
A208. The Linker^{A} of any one of embodiments A1-A193, wherein R¹⁶ is heterocycle.
A209. The Linker^{A} of embodiment A208, wherein R¹⁶ is piperidinyl.
A210. The Linker^{A} of embodiment A209, wherein R¹⁶ is

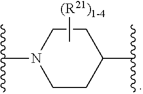

A211. The Linker^{A} of embodiment A209, wherein R¹⁶ is

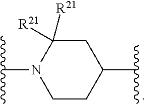

A212. The Linker^{A} of embodiment A208, wherein R¹⁶ is piperizinyl.
A213. The Linker^{A} of embodiment A212, wherein R¹⁶ is

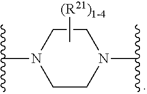

A214. The Linker^{A} of embodiment A208, wherein R¹⁶ is pyrrolidinyl.
A215. The Linker^{A} of embodiment A214, wherein R¹⁶ is

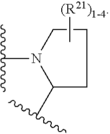

A216. The Linker^{A} of embodiment A214, wherein R¹⁶ is

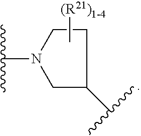

A217. The Linker^{A} of any one of embodiments A1-A193, wherein R¹⁶ is heteroaryl.
A218. The Linker^{A} of embodiment A217, wherein R¹⁶ is pyridinyl.
A219. The Linker^{A} of embodiment A218, wherein R¹⁶ is

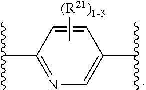

A220. The Linker^{A} of embodiment A218, wherein R¹⁶ is

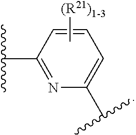

A221. The Linker^{A} of embodiment A218, wherein R¹⁶ is

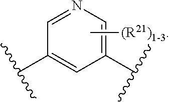

A222. The Linker^{A} of embodiment A217, wherein R¹⁶ is pyrimidinyl.
A223. The Linker^{A} of embodiment A222, wherein R¹⁶ is

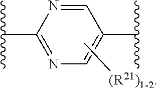

A224. The Linker^{A} of embodiment A222, wherein R¹⁶ is

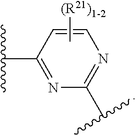

A225. The Linker^{A} of embodiment A217, wherein R¹⁶ is pyrazinyl.
A226. The Linker^{A} of embodiment A225, wherein R¹⁶ is

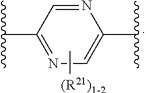

A227. The Linker^{A} of embodiment A225, wherein R¹⁶ is

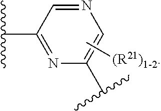

A228. The Linker^{A} of embodiment A217, wherein R¹⁶ is pyridizinyl.
A229. The Linker^{A} of embodiment A228, wherein R¹⁶ is

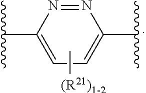

A230. The Linker^{A} of embodiment A228, wherein R¹⁶ is

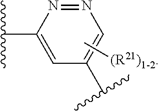

A231. The Linker^{A} of any one of embodiments A1-A193, wherein R¹⁶ is alkynyl.
A232. The Linker^{A} of any one of embodiments A1-A193, wherein R¹⁶ is alkoxy.
A233. The Linker^{A} of any one of embodiments A1-A232, wherein R¹⁷ is bond.
A234. The Linker^{A} of any one of embodiments A1-A232, wherein R¹⁷ is alkyl.
A235. The Linker^{A} of any one of embodiments A1-A232, wherein R¹⁷ is —C(O)—.
A236. The Linker^{A} of any one of embodiments A1-A232, wherein R¹⁷ is —C(O)O—.
A237. The Linker^{A} of any one of embodiments A1-A232, wherein R¹⁷ is —C(O)NR⁶—.
A238. The Linker^{A} of any one of embodiments A1-A232, wherein R¹⁷ is —NR⁶C(O)—.
A239. The Linker^{A} of any one of embodiments A1-A232, wherein R¹⁷ is —NR⁶—.
A240. The Linker^{A} of any one of embodiments A1-A232, wherein R¹⁷ is —O—.
A241. The Linker^{A} of any one of embodiments A1-A232, wherein R¹⁷ is —C(R²¹R²¹)—.
A242. The Linker^{A} of embodiment A241, wherein both R²¹ groups are hydrogen.
A243. The Linker^{A} of any one of embodiments A1-A232, wherein R¹⁷ is —CH₂CH₂—[O—(CH₂)₂]ₙ—O—.
A244. The Linker^{A} of any one of embodiments A1-A232, wherein R¹⁷ is aryl.
A245. The Linker^{A} of embodiment A244, wherein R¹⁷ is phenyl.
A246. The Linker^{A} of embodiment A244, wherein R¹⁷ is naphthyl.
A247. The Linker^{A} of any one of embodiments A1-A232, wherein R¹⁷ is heterocycle.
A248. The Linker^{A} of embodiment A247, wherein R¹⁷ is piperidinyl.
A249. The Linker^{A} of embodiment A248, wherein R¹⁷ is

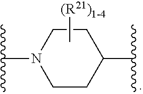

A250. The Linker^{A} of embodiment A248, wherein R¹⁷ is

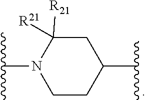

A251. The Linker^{A} of embodiment A247, wherein R¹⁷ is piperizinyl.
A252. The Linker^{A} of embodiment A251, wherein R¹⁷ is

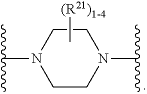

A253. The Linker^{A} of embodiment A247, wherein R¹⁷ is pyrrolidinyl.
A254. The Linker^{A} of embodiment A253, wherein R¹⁷ is

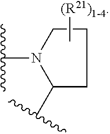

A255. The Linker^{A} of embodiment A253, wherein R¹⁷ is

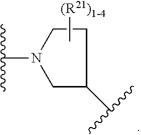

A256. The Linker^{A} of any one of embodiments A1-A232, wherein R¹⁷ is heteroaryl.
A257. The Linker^{A} of embodiment A256, wherein R¹⁷ is pyridinyl.
A258. The Linker^{A} of embodiment A257, wherein R¹⁷ is

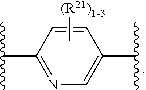

A259. The Linker^{A} of embodiment A257, wherein R¹⁷ is

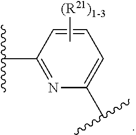

A260. The Linker^{A} of embodiment A257, wherein R¹⁷ is

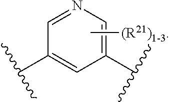

A261. The Linker^{A} of embodiment A256, wherein R¹⁷ is pyrimidinyl.
A262. The Linker^{A} of embodiment A261, wherein R¹⁷ is

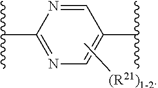

A263. The Linker^{A} of embodiment A261, wherein R¹⁷ is

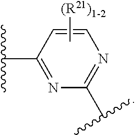

A264. The Linker^{A} of embodiment A256, wherein R¹⁷ is pyrazinyl.
A265. The Linker^{A} of embodiment A264, wherein R¹⁷ is

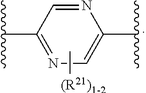

A266. The Linker^{A} of embodiment A264, wherein R¹⁷ is

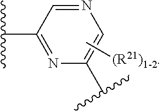

A267. The Linker^{A} of embodiment A256, wherein R¹⁷ is pyridizinyl.
A268. The Linker^{A} of embodiment A267, wherein R¹⁷ is

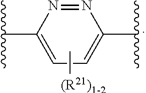

A269. The Linker^{A} of embodiment A267, wherein R¹⁷ is

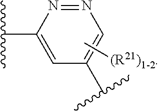

A270. The Linker^{A} of any one of embodiments A1-A232, wherein R¹⁷ is alkynyl.
A271. The Linker^{A} of any one of embodiments A1-A232, wherein R¹⁷ is alkoxy.
A272. The Linker^{A} of any one of embodiments A1-A271, wherein R¹⁸ is bond.
A273. The Linker^{A} of any one of embodiments A1-A271, wherein R¹⁸ is alkyl.
A274. The Linker^{A} of any one of embodiments A1-A271, wherein R¹⁸ is —C(O)—.
A275. The Linker^{A} of any one of embodiments A1-A271, wherein R¹⁸ is —C(O)O—.
A276. The Linker^{A} of any one of embodiments A1-A271, wherein R¹⁸ is —C(O)NR⁶—.
A277. The Linker^{A} of any one of embodiments A1-A271, wherein R¹⁸ is —NR⁶C(O)—.
A278. The Linker^{A} of any one of embodiments A1-A271, wherein R¹⁸ is —S(O)—.
A279. The Linker^{A} of any one of embodiments A1-A271, wherein R¹⁸ is —S(O)₂—.
A280. The Linker^{A} of any one of embodiments A1-A271, wherein R¹⁸ is —C(R²¹R²¹)—.
A281. The Linker^{A} of embodiment A280, wherein both R²¹ groups are hydrogen.
A282. The Linker^{A} of any one of embodiments A1-A271, wherein R¹⁸ is —CH₂CH₂—[O—(CH₂)₂]ₙ—O—.
A283. The Linker^{A} of any one of embodiments A1-A271, wherein R⁸ is aryl.
A284. The Linker^{A} of embodiment A283, wherein R¹⁸ is phenyl.
A285. The Linker^{A} of embodiment A283, wherein R¹⁸ is naphthyl.
A286. The Linker^{A} of any one of embodiments A1-A271, wherein R⁸ is heterocycle.
A287. The Linker^{A} of embodiment A286, wherein R¹⁸ is piperidinyl.
A288. The Linker^{A} of embodiment A287, wherein R¹⁸ is

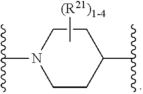

A289. The Linker^{A} of embodiment A287, wherein R¹⁸ is

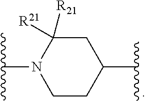

A290. The Linker^{A} of embodiment A286, wherein R¹⁸ is piperizinyl.
A291. The Linker^{A} of embodiment A290, wherein R¹⁸ is

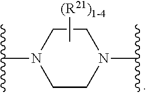

A292. The Linker^{A} of embodiment A286, wherein R¹⁸ is pyrrolidinyl.
A293. The Linker^{A} of embodiment A292, wherein R¹⁸ is

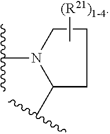

A294. The Linker^{A} of embodiment A292, wherein R¹⁸ is

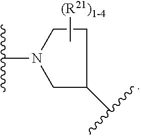

A295. The Linker^{A} of any one of embodiments A1-A271, wherein R⁸ is heteroaryl.
A296. The Linker^{A} of embodiment A295, wherein R¹⁸ is pyridinyl.
A297. The Linker^{A} of embodiment A296, wherein R¹⁸ is

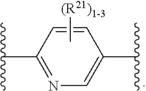

A298. The Linker^{A} of embodiment A296, wherein R¹⁸ is

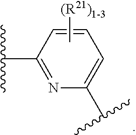

A299. The Linker^{A} of embodiment A296, wherein R¹⁸ is

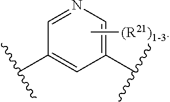

A300. The Linker^{A} of embodiment A295, wherein R¹⁸ is pyrimidinyl.
A301. The Linker^{A} of embodiment A300, wherein R¹⁸ is

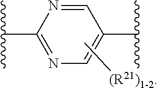

A302. The Linker^{A} of embodiment A300, wherein R¹⁸ is

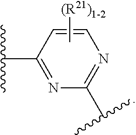

A303. The Linker^{A} of embodiment A295, wherein R¹⁸ is pyrazinyl.
A304. The Linker^{A} of embodiment A303, wherein R¹⁸ is

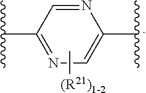

A305. The Linker^{A} of embodiment A303, wherein R¹⁸ is

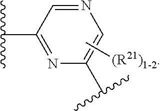

A306. The Linker^{A} of embodiment A295, wherein R¹⁸ is pyridizinyl.
A307. The Linker^{A} of embodiment A306, wherein R¹⁸ is

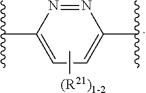

A308. The Linker^{A} of embodiment A306, wherein R¹⁸ is

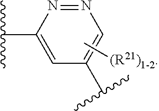

A309. The Linker^{A} of any one of embodiments A1-A271, wherein R¹⁸ is alkynyl.
A310. The Linker^{A} of any one of embodiments A1-A271, wherein R¹⁸ is alkoxy.
A311. The Linker^{A} of any one of embodiments A1-A310, wherein R¹⁹ is bond.
A312. The Linker^{A} of any one of embodiments A1-A310, wherein R¹⁹ is alkyl.
A313. The Linker^{A} of any one of embodiments A1-A310, wherein R¹⁹ is —C(O)—.
A314. The Linker^{A} of any one of embodiments A1-A310, wherein R¹⁹ is —C(O)O—.
A315. The Linker^{A} of any one of embodiments A1-A310, wherein R¹⁹ is —C(O)NR⁶—.
A316. The Linker^{A} of any one of embodiments A1-A310, wherein R¹⁹ is —NR⁶C(O)—.
A317. The Linker^{A} of any one of embodiments A1-A310, wherein R¹⁹ is —NR⁶—.
A318. The Linker^{A} of any one of embodiments A1-A310, wherein R¹⁹ is —O—.
A319. The Linker^{A} of any one of embodiments A1-A310, wherein R¹⁹ is —C(R²¹R²¹)—.
A320. The Linker^{A} of embodiment A319, wherein both R²¹ groups are hydrogen.
A321. The Linker^{A} of any one of embodiments A1-A310, wherein R¹⁹ is —CH₂CH₂—[O—(CH₂)₂]ₙ—O—.
A322. The Linker^{A} of any one of embodiments A1-A310, wherein R¹⁹ is aryl.
A323. The Linker^{A} of embodiment A322, wherein R¹⁹ is phenyl.
A324. The Linker^{A} of embodiment A322, wherein R¹⁹ is naphthyl.
A325. The Linker^{A} of any one of embodiments A1-A310, wherein R¹⁹ is heterocycle.
A326. The Linker^{A} of embodiment A325, wherein R¹⁹ is piperidinyl.
A327. The Linker^{A} of embodiment A326, wherein R¹⁹ is

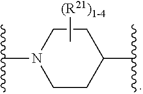

A328. The Linker^{A} of embodiment A326, wherein R¹⁹ is

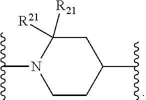

A329. The Linker^{A} of embodiment A325, wherein R¹⁹ is piperizinyl.
A330. The Linker^{A} of embodiment A329, wherein R¹⁹ is

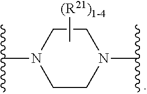

A331. The Linker^{A} of embodiment A325, wherein R¹⁹ is pyrrolidinyl.

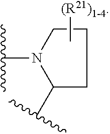

A332. The Linker^{A} of embodiment A331, wherein R¹⁹ is

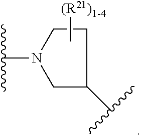

A333. The Linker^{A} of embodiment A331, wherein R¹⁹ is.
A334. The Linker^{A} of any one of embodiments A1-A310, wherein R¹⁹ is heteroaryl.
A335. The Linker^{A} of embodiment A334, wherein R¹⁹ is pyridinyl.
A336. The Linker^{A} of embodiment A335, wherein R¹⁹ is

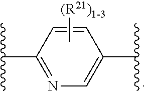

A337. The Linker^{A} of embodiment A335, wherein R¹⁹ is

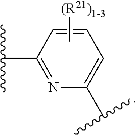

A338. The Linker^{A} of embodiment A335, wherein R¹⁹ is

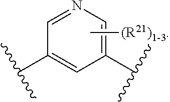

A339. The Linker^{A} of embodiment A334, wherein R¹⁹ is pyrimidinyl.
A340. The Linker^{A} of embodiment A339, wherein R¹⁹ is

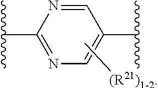

A341. The Linker^{A} of embodiment A339, wherein R¹⁹ is

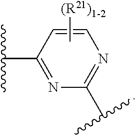

A342. The Linker^{A} of embodiment A334, wherein R¹⁹ is pyrazinyl.
A343. The Linker^{A} of embodiment A342, wherein R¹⁹ is

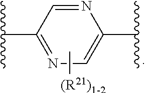

A344. The Linker^{A} of embodiment A342, wherein R¹⁹ is

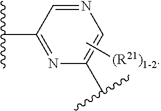

A345. The Linker^{A} of embodiment A334, wherein R¹⁹ is pyridizinyl.
A346. The Linker^{A} of embodiment A345, wherein R¹⁹ is

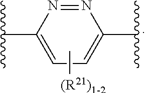

A347. The Linker^{A} of embodiment A345, wherein R¹⁹ is

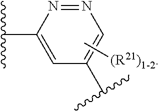

A348. The Linker^{A} of any one of embodiments A1-A310, wherein R¹⁹ is alkynyl.
A349. The Linker^{A} of any one of embodiments A1-A310, wherein R¹⁹ is alkoxy.
A350. The Linker^{A} of any one of embodiments A1-A349, wherein R²⁰ is bond.
A351. The Linker^{A} of any one of embodiments A1-A349, wherein R²⁰ is alkyl.
A352. The Linker^{A} of any one of embodiments A1-A349, wherein R²⁰ is —C(O)—.
A353. The Linker^{A} of any one of embodiments A1-A349, wherein R²⁰ is —C(O)O—.
A354. The Linker^{A} of any one of embodiments A1-A349, wherein R²⁰ is —C(O)NR⁶—.
A355. The Linker^{A} of any one of embodiments A1-A349, wherein R²⁰ is —NR⁶C(O)—.
A356. The Linker^{A} of any one of embodiments A1-A349, wherein R²⁰ is —S(O)—.
A357. The Linker^{A} of any one of embodiments A1-A349, wherein R²⁰ is —S(O)₂—.
A358. The Linker^{A} of any one of embodiments A1-A349, wherein R²⁰ is —C(R²¹R²¹)-A359. The Linker^{A} of embodiment A358, wherein both R²¹ groups are hydrogen.
A360. The Linker^{A} of any one of embodiments A1-A349, wherein R²⁰ is —CH₂CH₂—[O—(CH₂)₂]ₙ—O—.
A361. The Linker^{A} of any one of embodiments A1-A349, wherein R²⁰ is aryl.
A362. The Linker^{A} of embodiment A361, wherein R²⁰ is phenyl.
A363. The Linker^{A} of embodiment A361, wherein R²⁰ is naphthyl.
A364. The Linker^{A} of any one of embodiments A1-A349, wherein R²⁰ is heterocycle.
A365. The Linker^{A} of embodiment A364, wherein R²⁰ is piperidinyl.
A366. The Linker^{A} of embodiment A364, wherein R²⁰ is

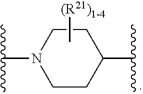

A367. The Linker^{A} of embodiment A364, wherein R²⁰ is

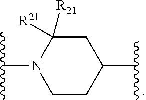

A368. The Linker^{A} of embodiment A364, wherein R²⁰ is piperizinyl.
A369. The Linker^{A} of embodiment A364, wherein R²⁰ is

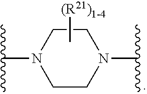

A370. The Linker^{A} of embodiment A364, wherein R²⁰ is pyrrolidinyl.
A371. The Linker^{A} of embodiment A364, wherein R²⁰ is

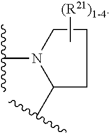

A372. The Linker^{A} of embodiment A364, wherein R²⁰ is

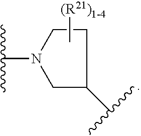

A373. The Linker^{A} of any one of embodiments A1-A349, wherein R²⁰ is heteroaryl.
A374. The Linker^{A} of embodiment A373, wherein R²⁰ is pyridinyl.
A375. The Linker^{A} of embodiment A373, wherein R²⁰ is

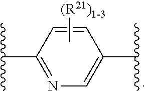

A376. The Linker^{A} of embodiment A373, wherein R²⁰ is

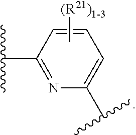

A377. The Linker^{A} of embodiment A373, wherein R²⁰ is

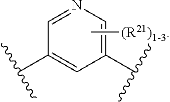

A378. The Linker^{A} of embodiment A373, wherein R²⁰ is pyrimidinyl.
A379. The Linker^{A} of embodiment A373, wherein R²⁰ is

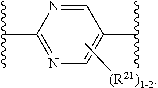

A380. The Linker^{A} of embodiment A373, wherein R²⁰ is

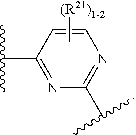

A381. The Linker^{A} of embodiment A373, wherein R²⁰ is pyrazinyl.

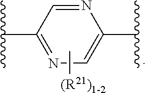

A383. The Linker^{A} of embodiment A373, wherein R²⁰ is

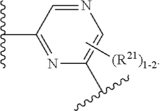

A384. The Linker^{A} of embodiment A373, wherein R²⁰ is pyridizinyl.
A385. The Linker^{A} of embodiment A373, wherein R²⁰ is

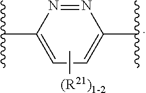

A386. The Linker^{A} of embodiment A373, wherein R²⁰ is

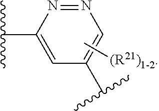

A387. The Linker^{A} of any one of embodiments A1-A349, wherein R²⁰ is alkynyl.
A388. The Linker^{A} of any one of embodiments A1-A349, wherein R²⁰ is alkoxy.
A389. The Linker^{A} of any one of embodiments A1-A388, wherein R¹¹, R¹², R¹³, R¹⁴, R¹, R¹⁶, R¹⁷, R¹⁸, R¹⁹, or R²⁰ is substituted with two R²¹ substituents, wherein the substituents are selected such that a stable compound results.
A390. The Linker^{A} of any one of embodiments A1-A388, wherein R¹¹, R¹², R¹³, R¹⁴, R¹, R¹⁶, R¹⁷, R¹⁸, R¹⁹, or R²⁰ is substituted with three R²¹ substituents, wherein the substituents are selected such that a stable compound results.
A391. The Linker^{A} of any one of embodiments A1-A388, wherein R¹¹, R¹², R¹³, R¹⁴, R¹, R¹⁶, R¹⁷, R¹⁸, R¹⁹, or R²⁰ is substituted with four R²¹ substituents, wherein the substituents are selected such that a stable compound results.
A392. The Linker^{A} of any one of embodiments A1-A388, wherein R¹¹, R¹², R¹³, R¹⁴, R¹⁵, R¹⁶, R¹⁷, R¹⁸, R¹⁹, or R²⁰ is substituted with one R²¹ substituent.
A393. The Linker^{A} of any one of embodiments A1-A392, wherein R²¹ is independently at each occurrence selected from the group consisting of hydrogen, alkyl, alkenyl, alkynyl, F, Cl, Br, I, hydroxyl, alkoxy, amino, —NR⁶R⁷, haloalkyl, aryl, heteroaryl, and heterocycle.
A394. The Linker^{A} of any one of embodiments A1-A393, R²¹ is independently at each occurrence selected from the group consisting of hydrogen, alkyl, F, hydroxyl, alkoxy, amino, —NR⁶R⁷, haloalkyl, aryl, heteroaryl, and heterocycle.
A395. The Linker^{A} of any one of embodiments A1-A394, R²¹ is independently at each occurrence selected from the group consisting of hydrogen, alkyl, F, hydroxyl, alkoxy, amino, —NR⁶R⁷, and haloalkyl.
A396. The Linker^{A} of any one of embodiments A1-A391, wherein one of the R²¹ groups is hydrogen.
A397. The Linker^{A} of any one of embodiments A1-A391, wherein one of the R²¹ groups is fluorine.
A398. The Linker^{A} of any one of embodiments A1-A391, wherein one of the R²¹ groups is alkyl.
A399. The Linker^{A} of embodiment A398, wherein one of the R²¹ groups is methyl.
A400. The Linker^{A} of embodiment A398, wherein one of the R²¹ groups is ethyl.
A401. The Linker^{A} of embodiment A398, wherein one of the R²¹ groups is isopropyl.
A402. The Linker^{A} of any one of embodiments A1-A391, wherein one of the R²¹ groups is hydroxyl.
A403. The Linker^{A} of any one of embodiments A1-A391, wherein one of the R²¹ groups is —NR⁶R⁷.
A404. The Linker^{A} of embodiment A403, wherein one of the R²¹ groups is dimethylamino.
A405. The Linker^{A} of any one of embodiments A1-A391 and A393-A398, wherein a second R²¹ group is hydrogen.
A406. The Linker^{A} of any one of embodiments A1-A391 and A393-A398, wherein a second R²¹ group is fluorine.
A407. The Linker^{A} of any one of embodiments A1-A391 and A393-A398, wherein a second R²¹ group is alkyl.
A408. The Linker^{A} of any one of embodiments A1-A388 and A390-A391, wherein a third R²¹ is hydrogen.
A409. The Linker^{A} of any one of embodiments A1-A388 and A390-A391, wherein a third R²¹ is fluorine.
A410. The Linker^{A} of any one of embodiments A1-A388 and A390-A391, wherein a third R²¹ is alkyl.
A411. The Linker^{A} of any one of embodiments A1-A388 and A391, wherein a fourth R²¹ is hydrogen

In certain embodiments Linker^{A} is selected from:

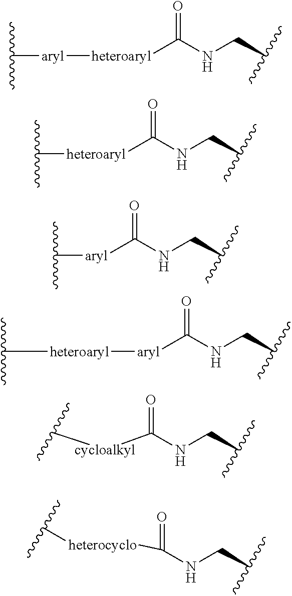

each of which is optionally substituted with 1, 2, 3, or 4 optional substituents as defined herein.

In certain embodiments Linker^{A} is selected from:

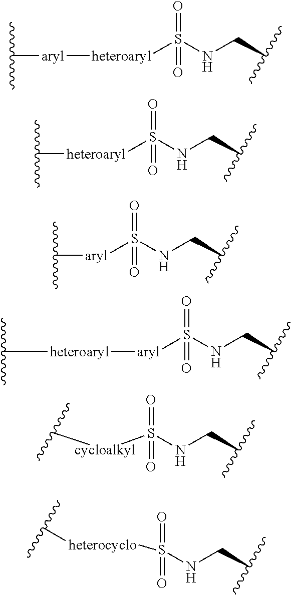

each of which is optionally substituted with 1, 2, 3, or 4 optional substituents as defined herein.

In certain embodiments Linker^{A} is selected from:

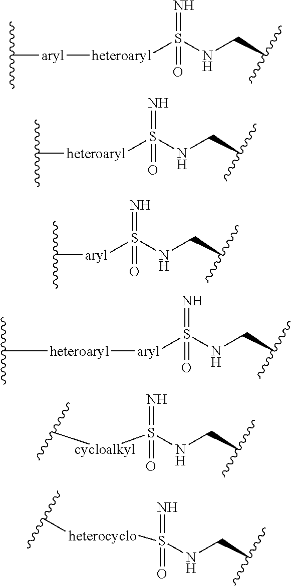

each of which is optionally substituted with 1, 2, 3, or 4 optional substituents as defined herein.

In certain embodiments Linker^{A} is selected from:

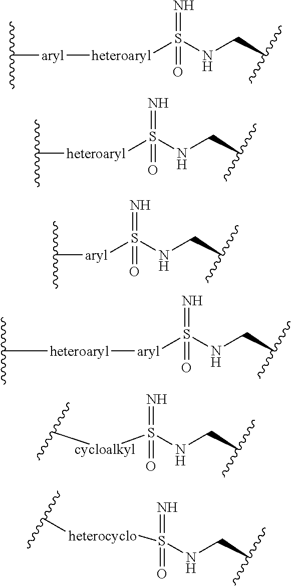

each of which is optionally substituted with 1, 2, 3, or 4 optional substituents as defined herein.

In certain embodiments Linker^{A} is selected from:

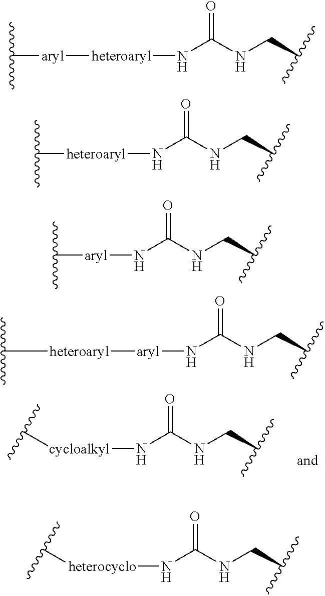

each of which is optionally substituted with 1, 2, 3, or 4 optional substituents as defined herein.

In certain embodiments Linker^{A} is selected from:

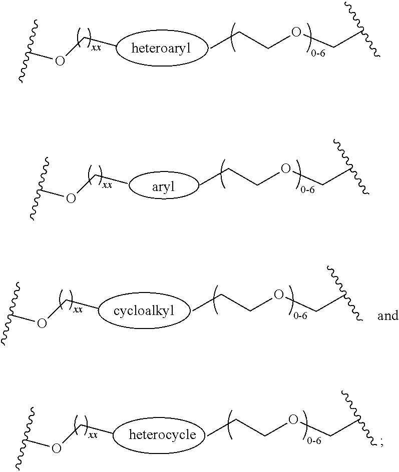

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

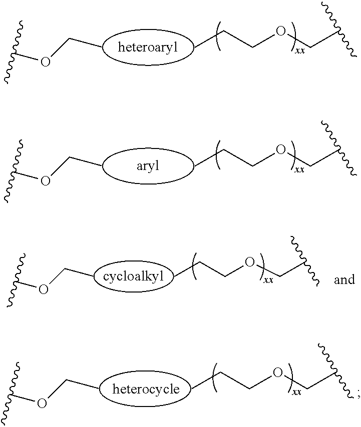

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

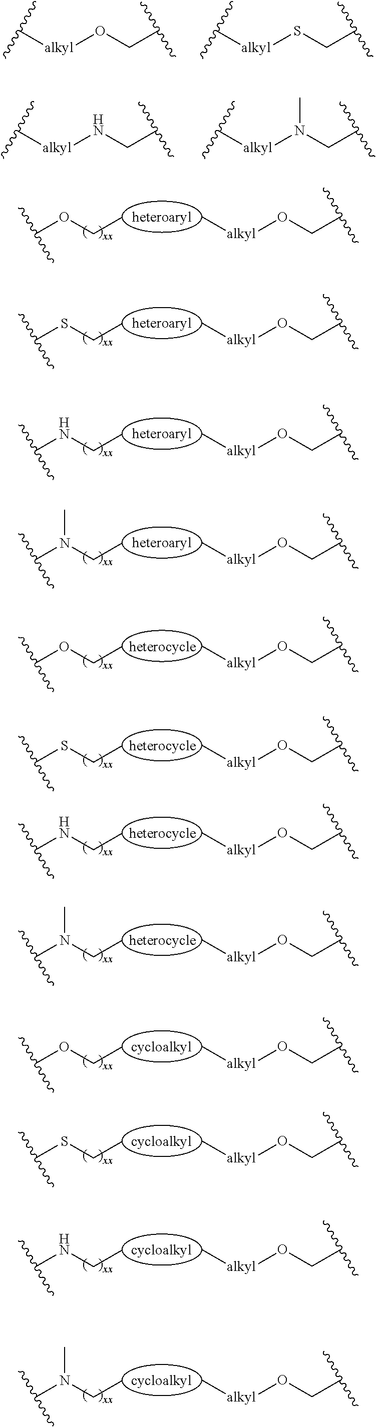

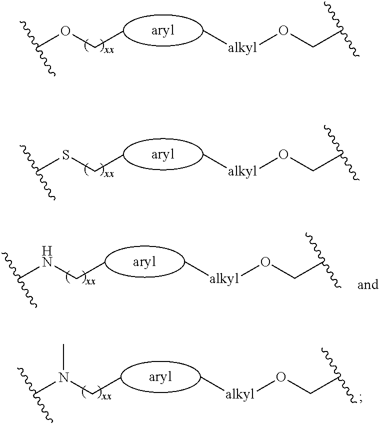

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

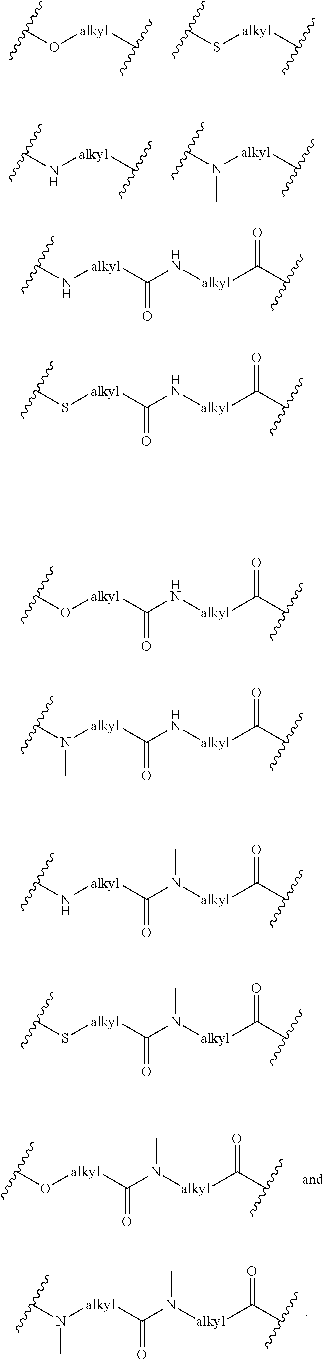

In certain embodiments Linker^{B} is selected from:

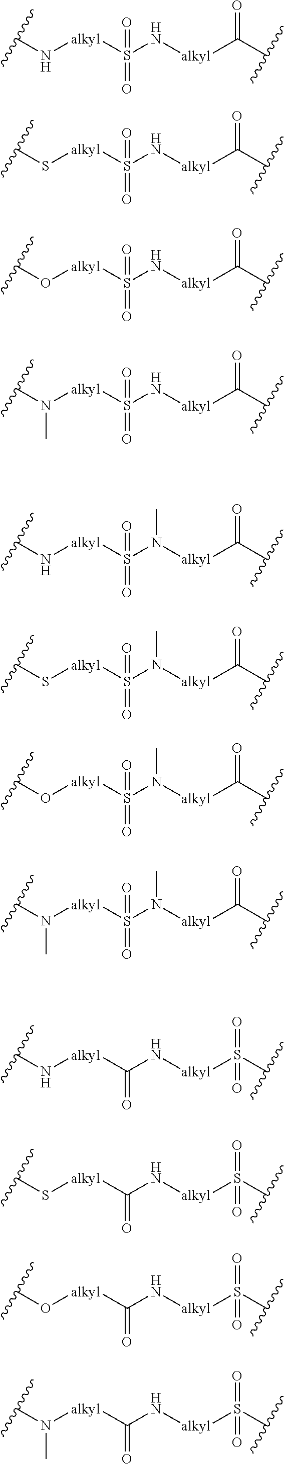

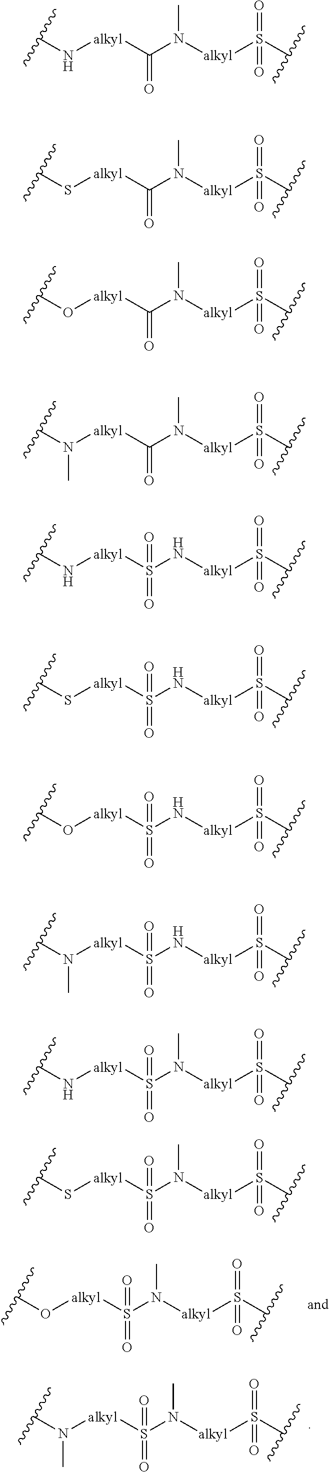

In certain embodiments Linker^{B}, Linker^{C}, or Linker^{D} is selected from:

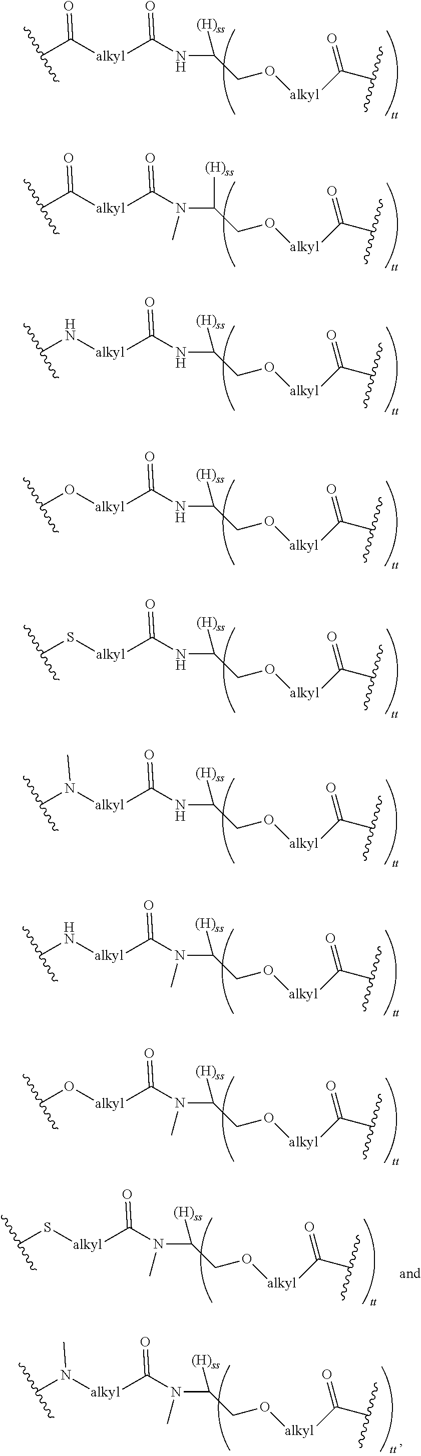

wherein tt is independently selected from 1, 2, or 3 and ss is 3 minus tt.

In certain embodiments Linker^{B}, Linker^{C}, or Linker^{D} is selected from:

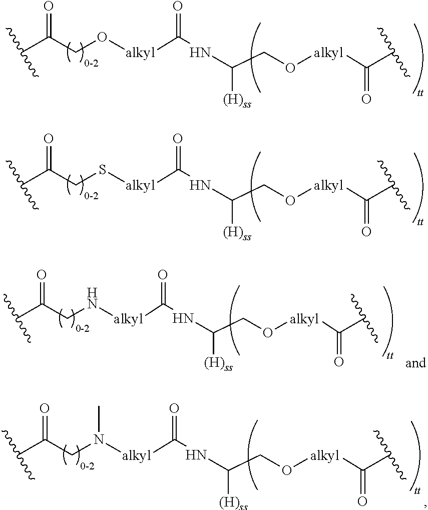

wherein tt and ss are as defined herein.

In certain embodiments Linker^{B}, Linker^{C}, or Linker^{D} is selected from:

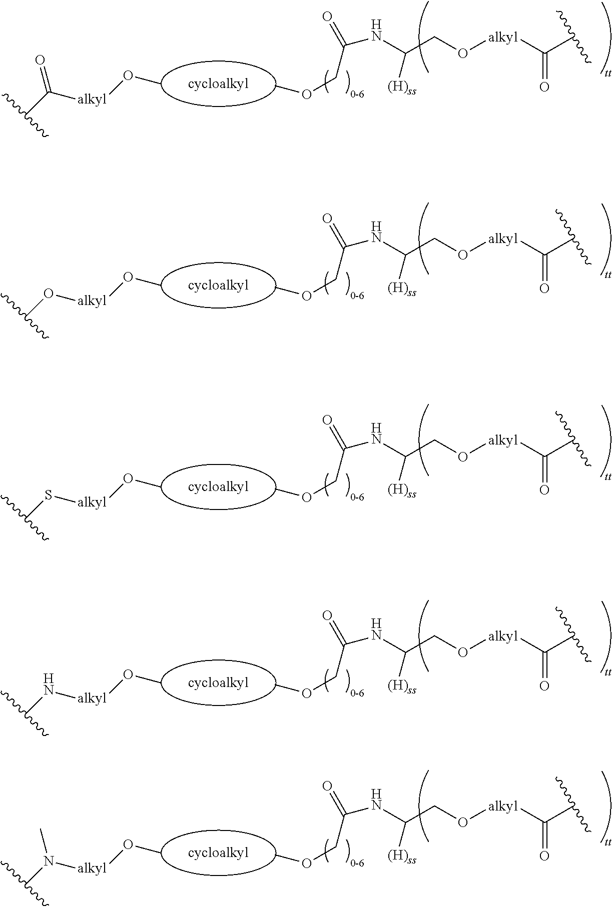

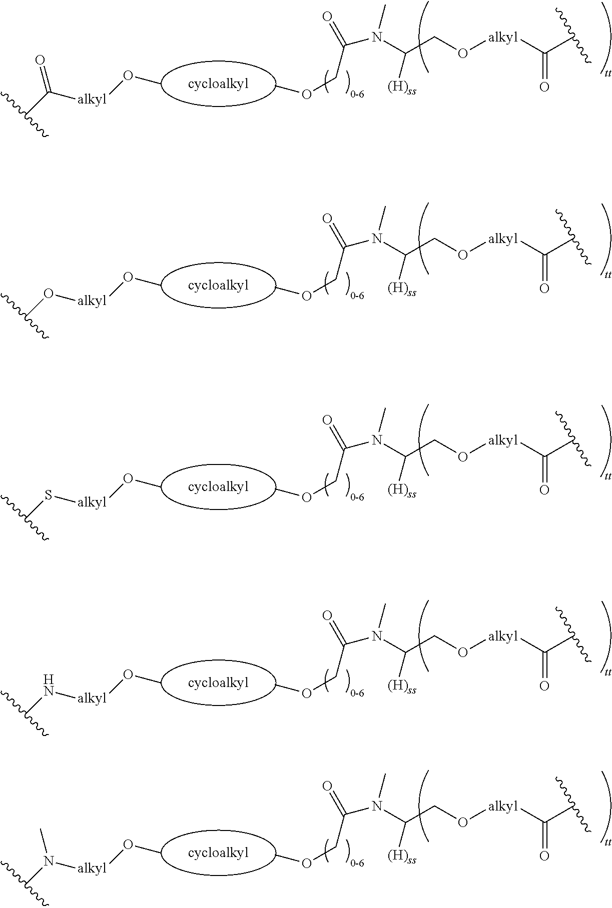

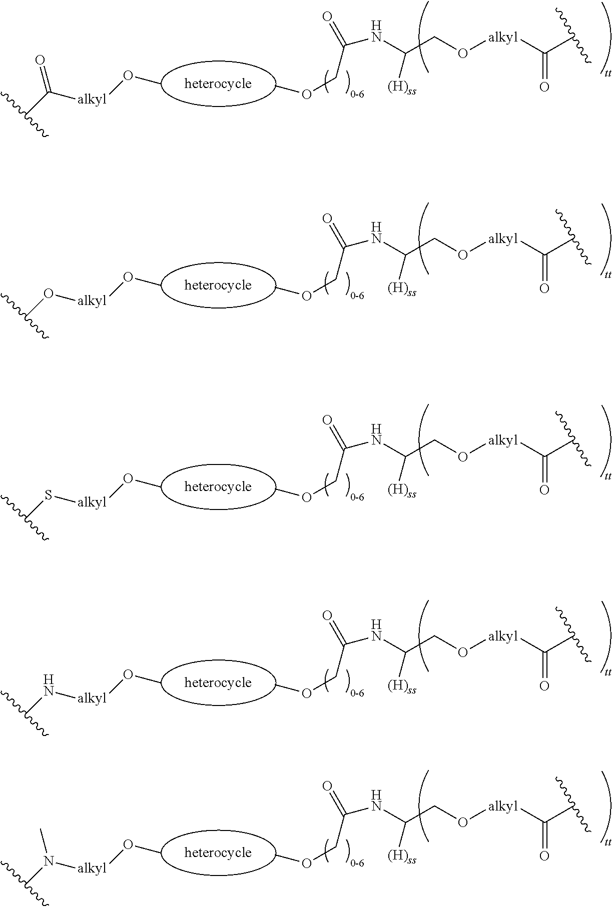

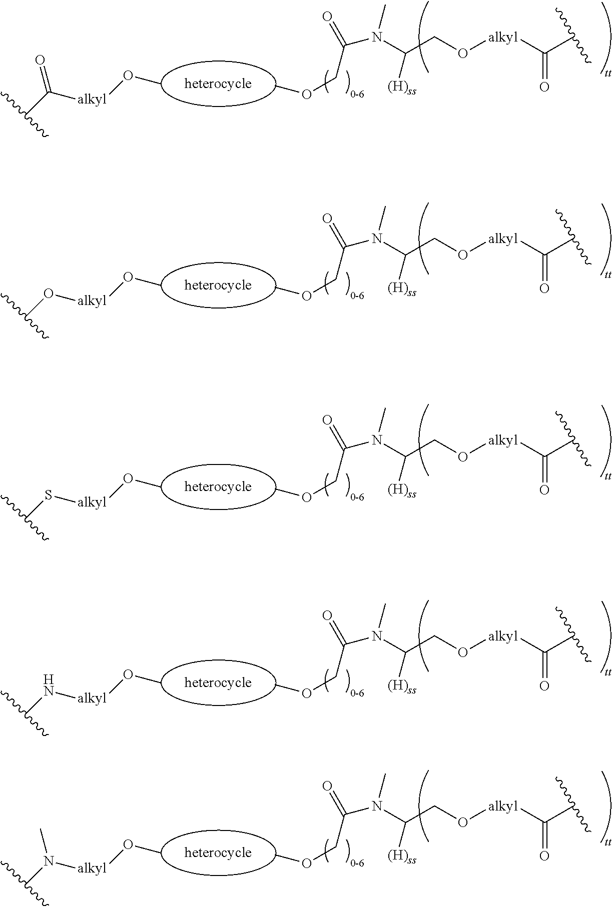

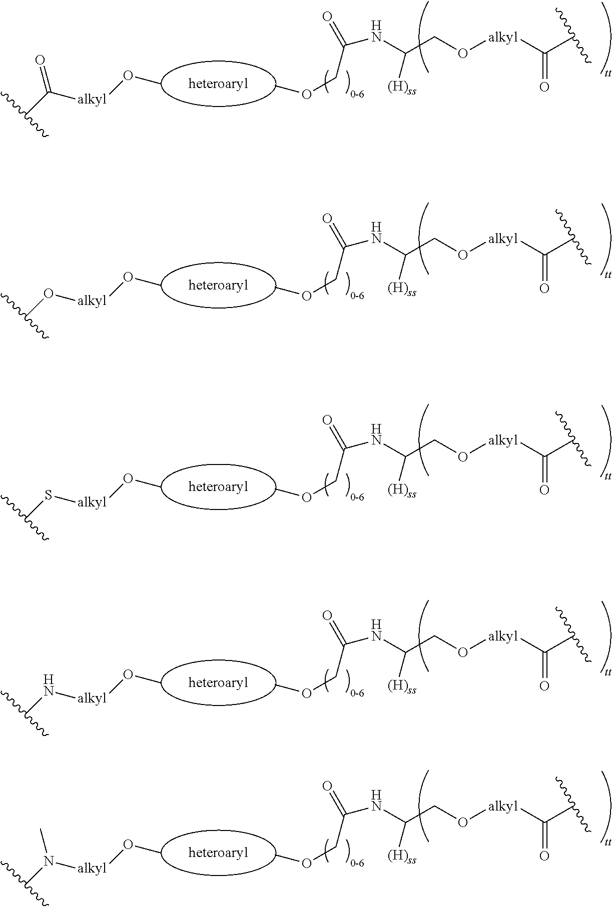

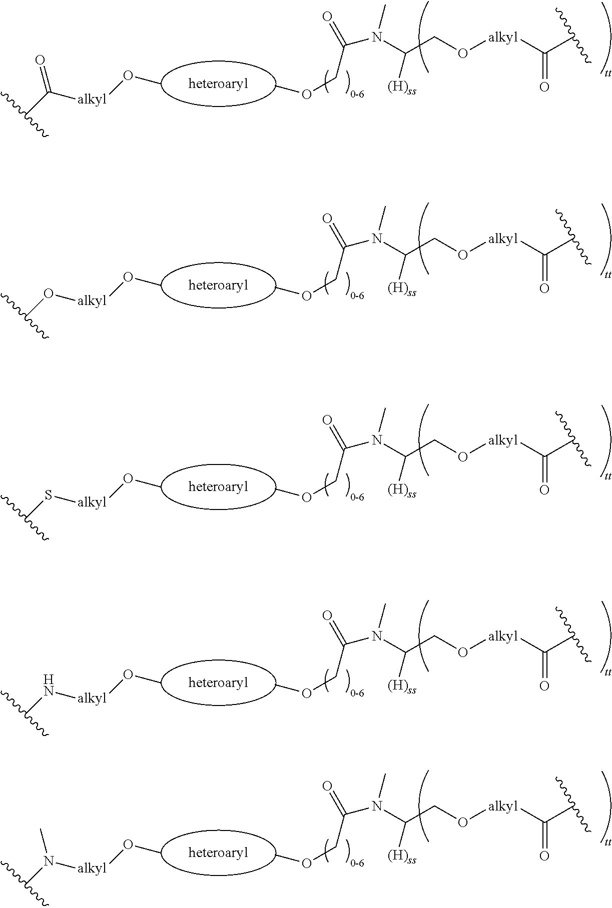

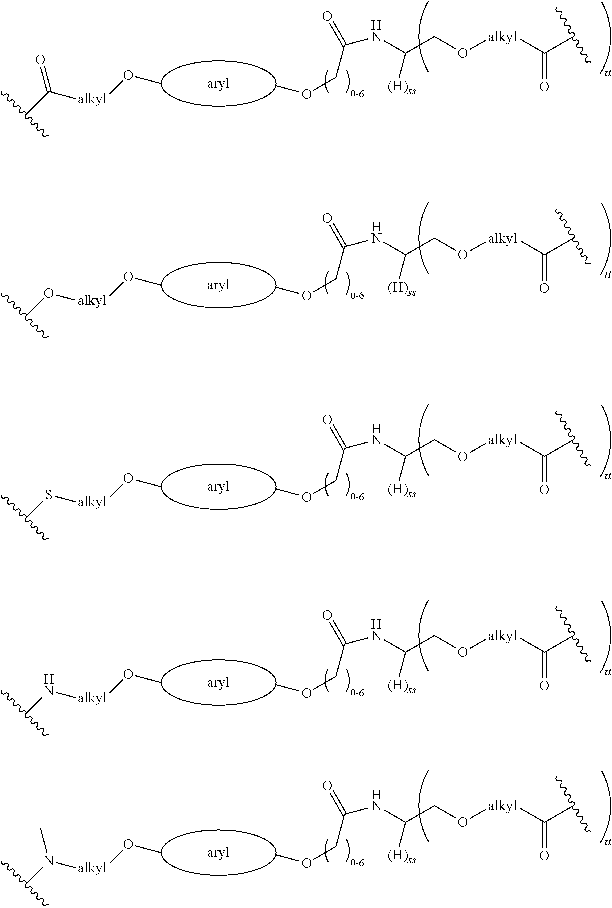

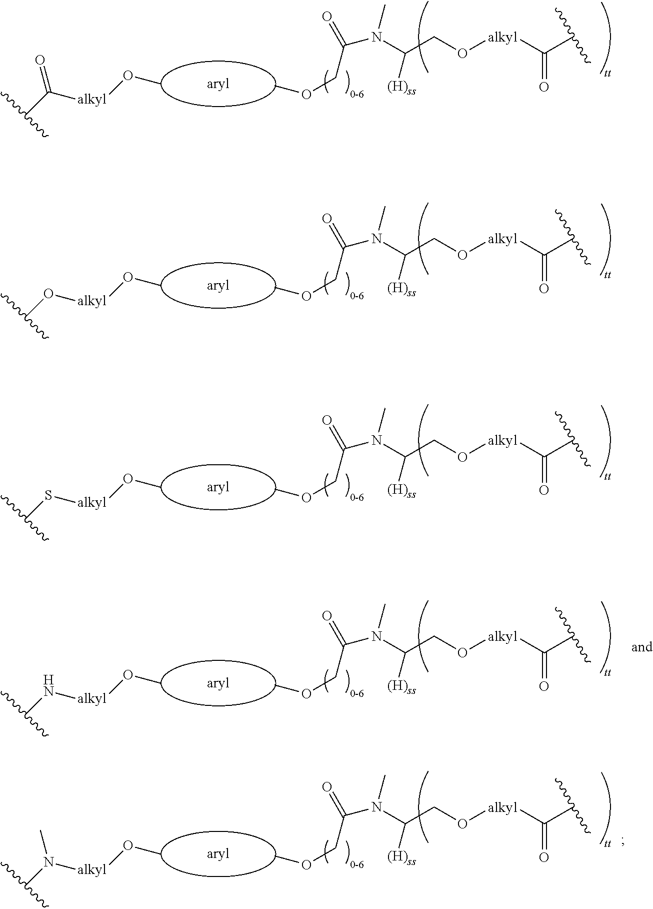

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

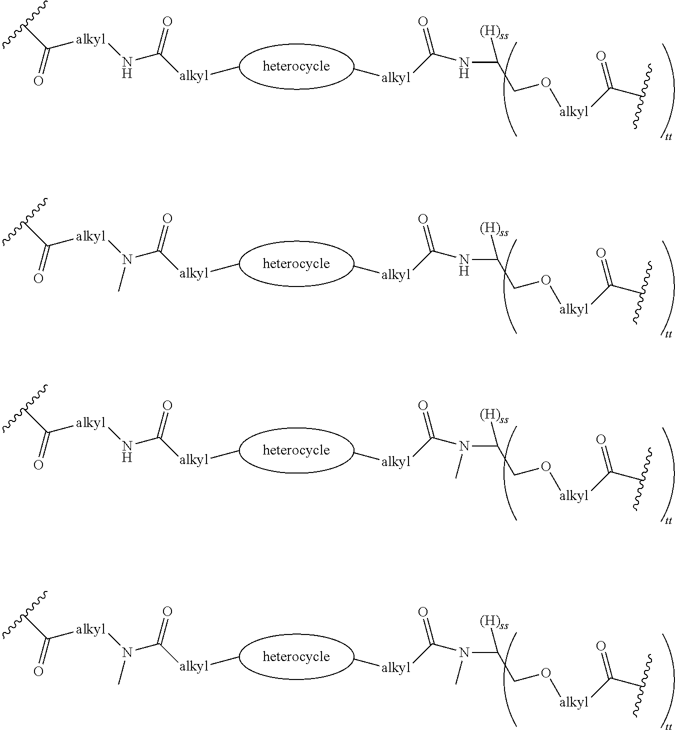

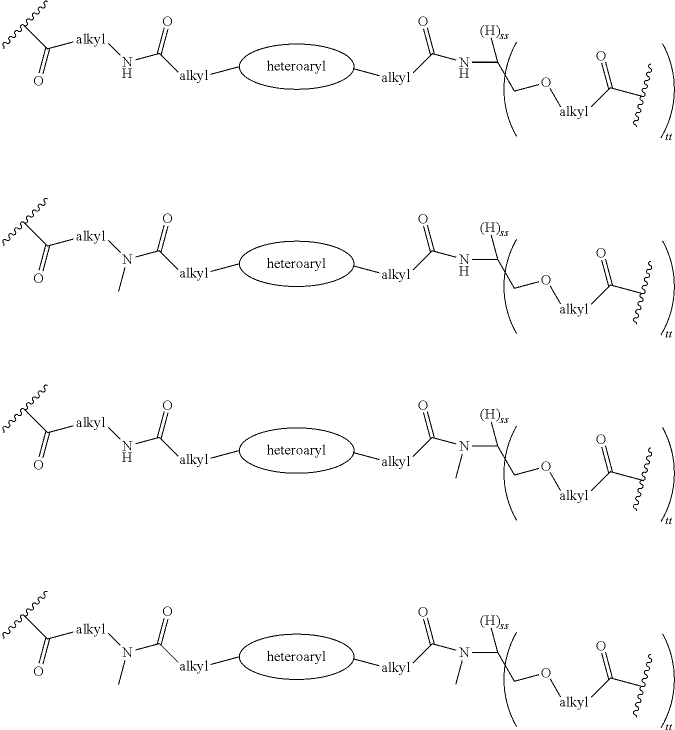

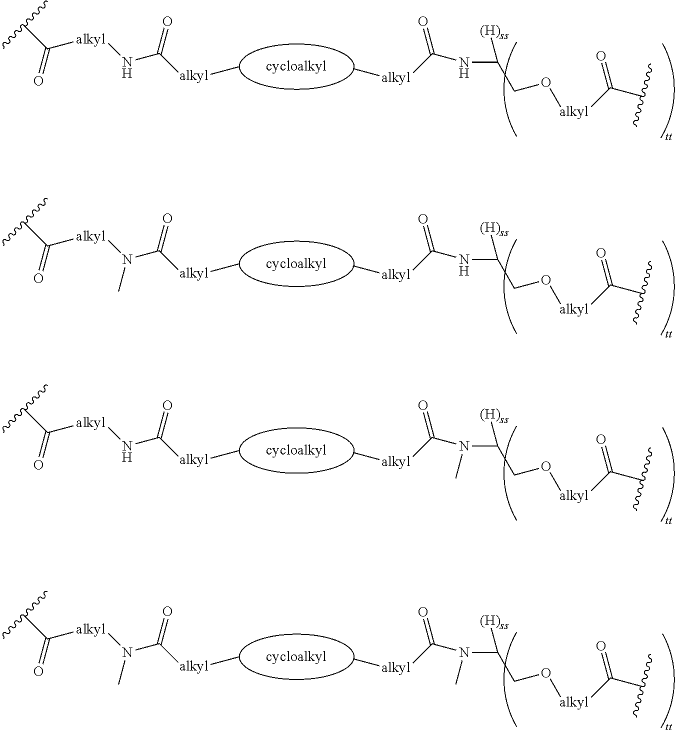

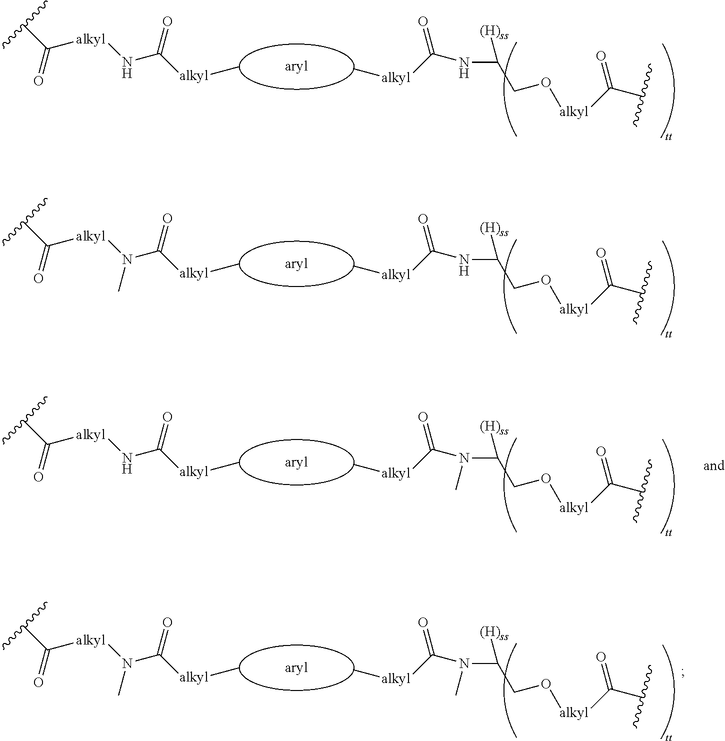

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

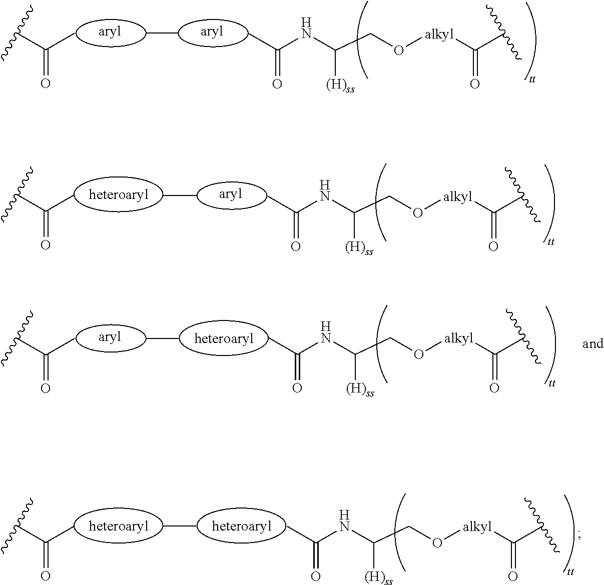

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{A} is selected from:

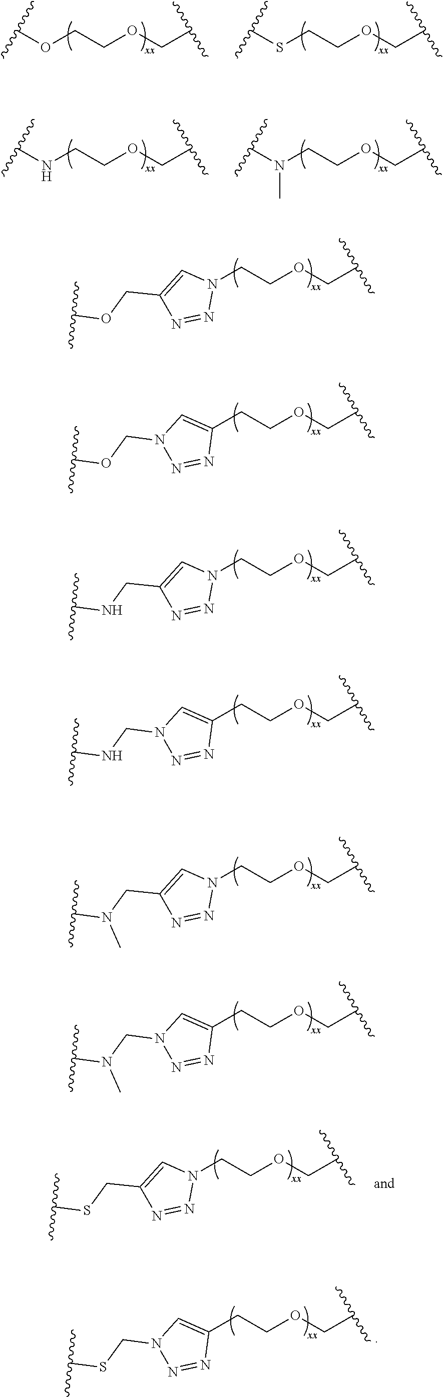

In certain embodiments Linker^{A} is selected from:

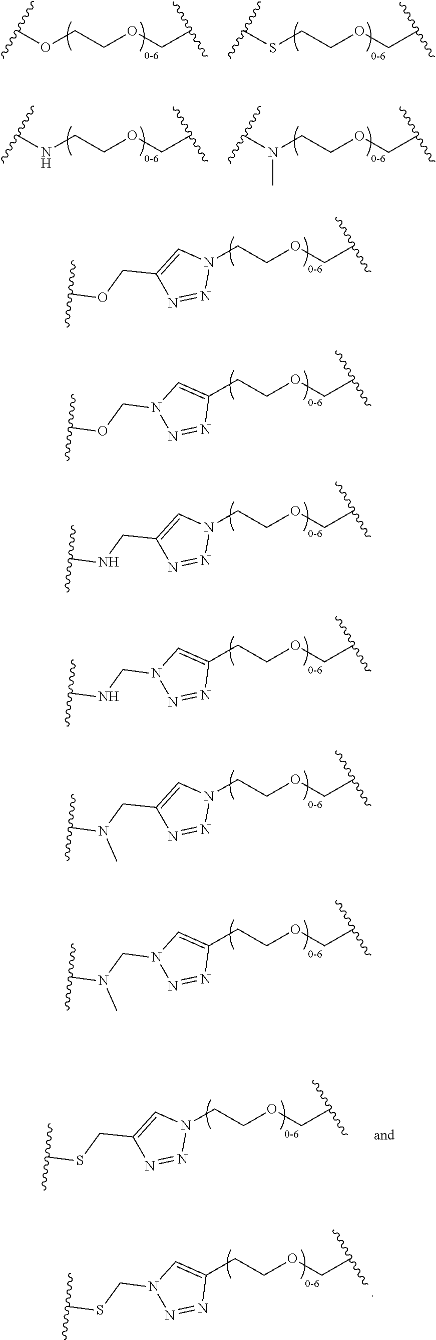

In certain embodiments Linker^{A} is selected from:

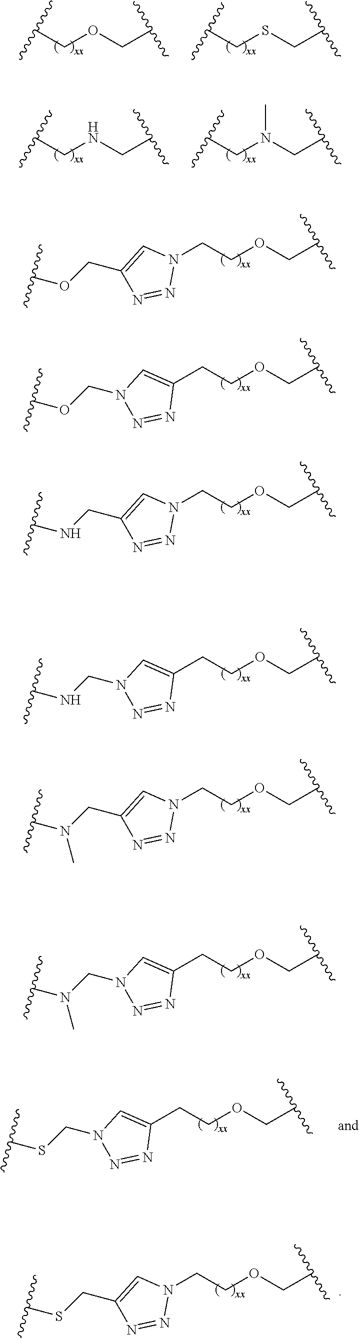

In certain embodiments Linker^{A} is selected from:

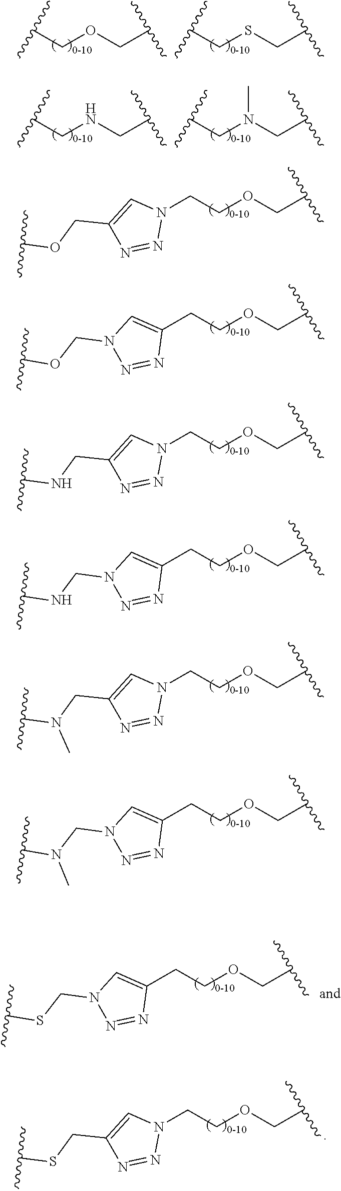

In certain embodiments Linker^{B} is selected from

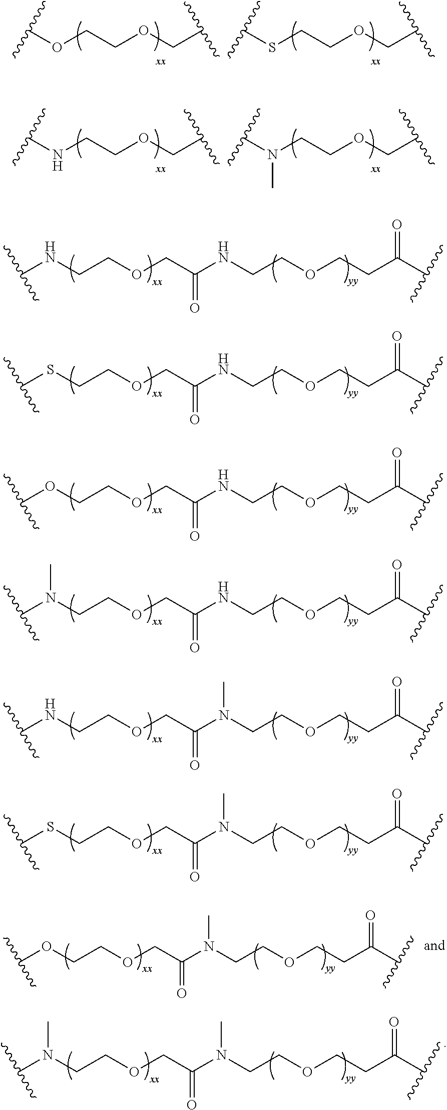

In certain embodiments Linker^{B} is selected from:

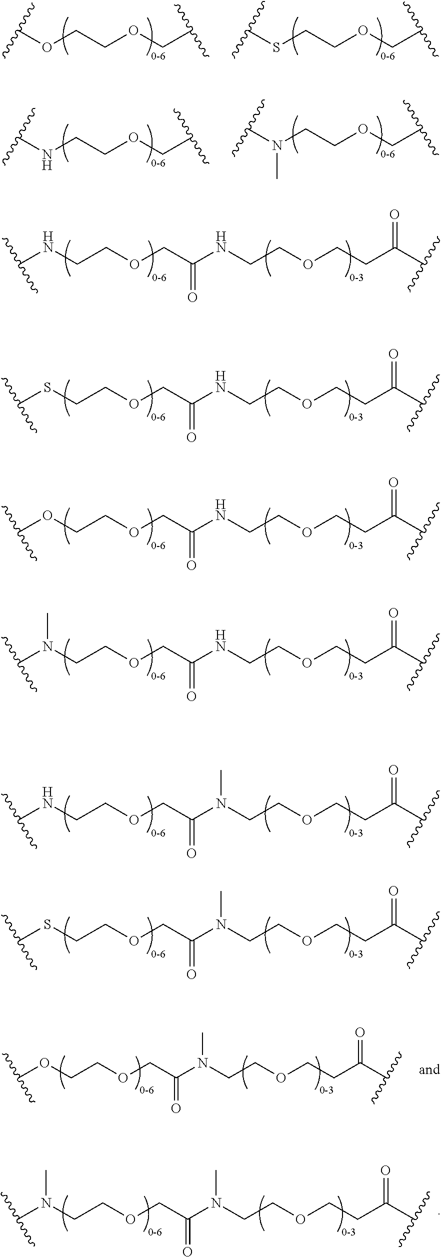

In certain embodiments Linker^{B} is selected from:

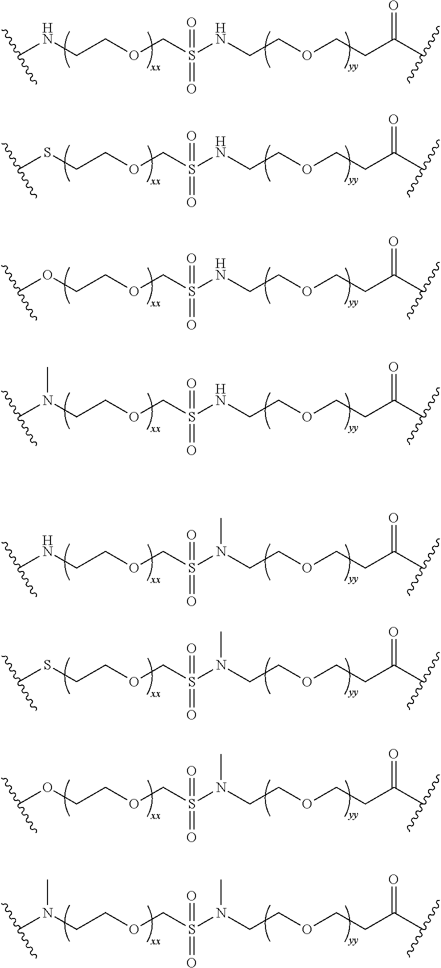

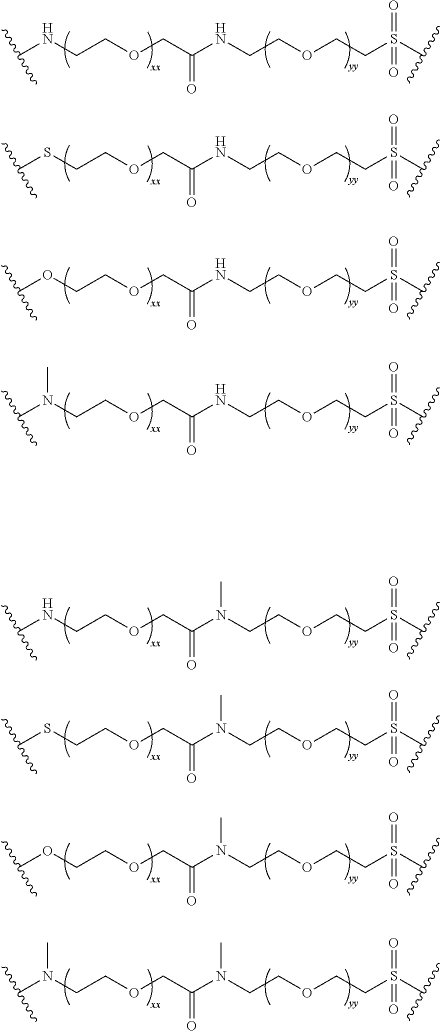

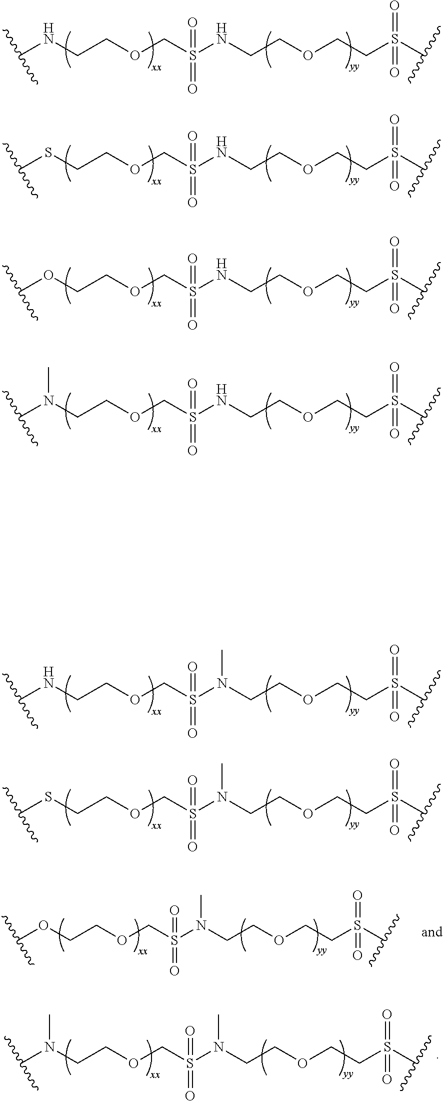

In certain embodiments Linker^{B} is selected from:

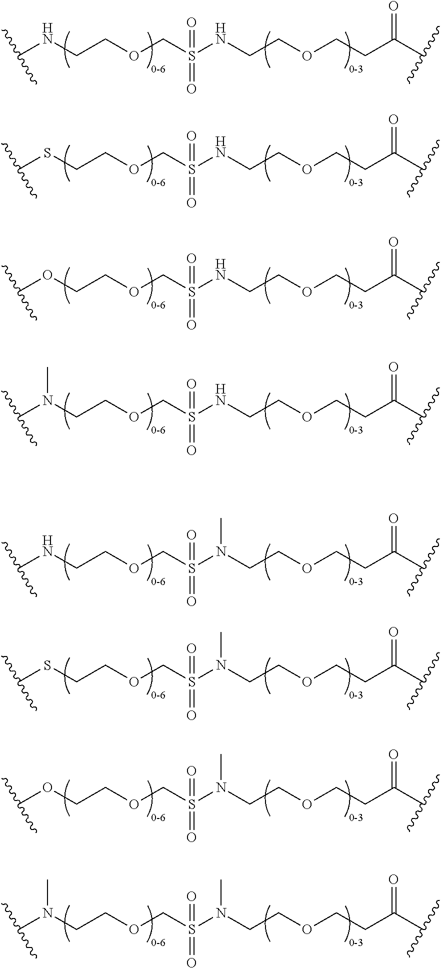

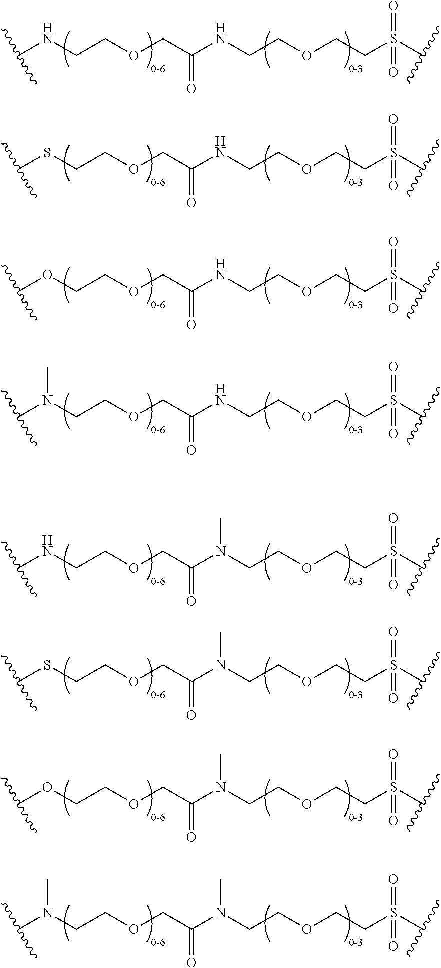

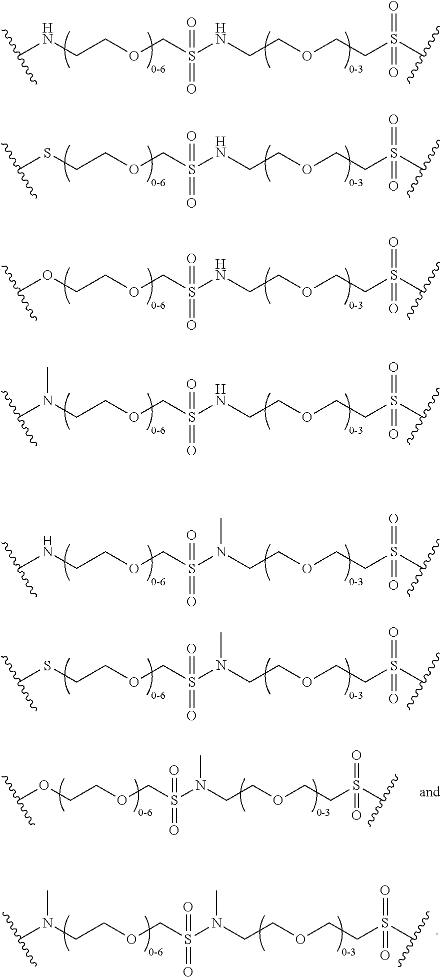

In certain embodiments Linker^{C} is selected from:

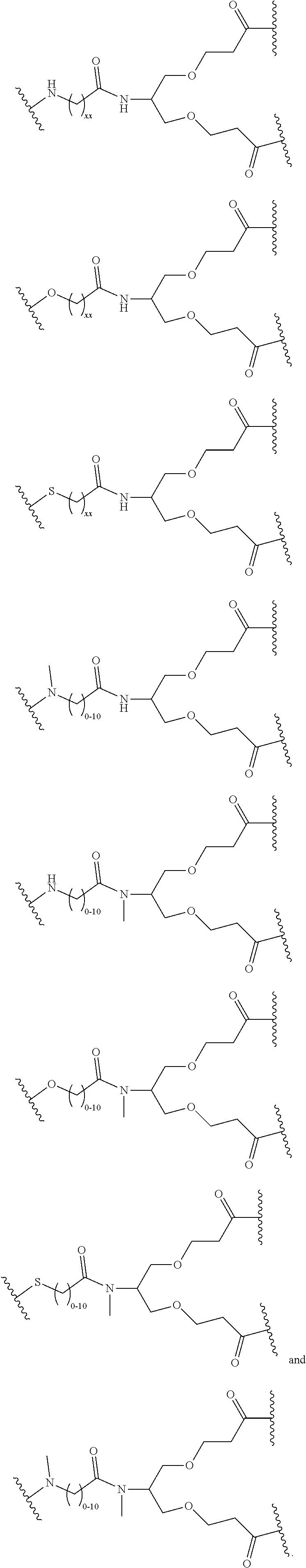

In certain embodiments Linker^{C} is selected from:

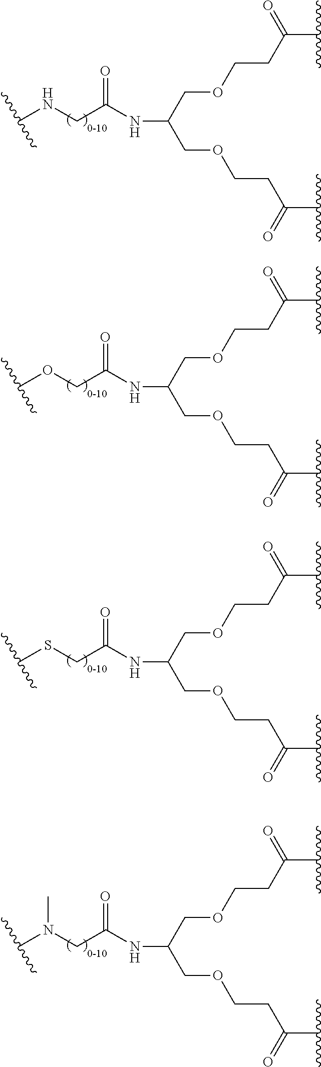

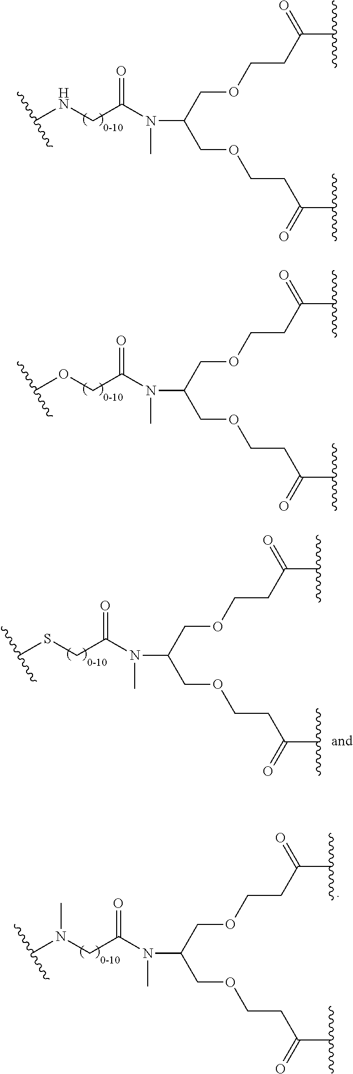

In certain embodiments Linker^{C} is selected from:

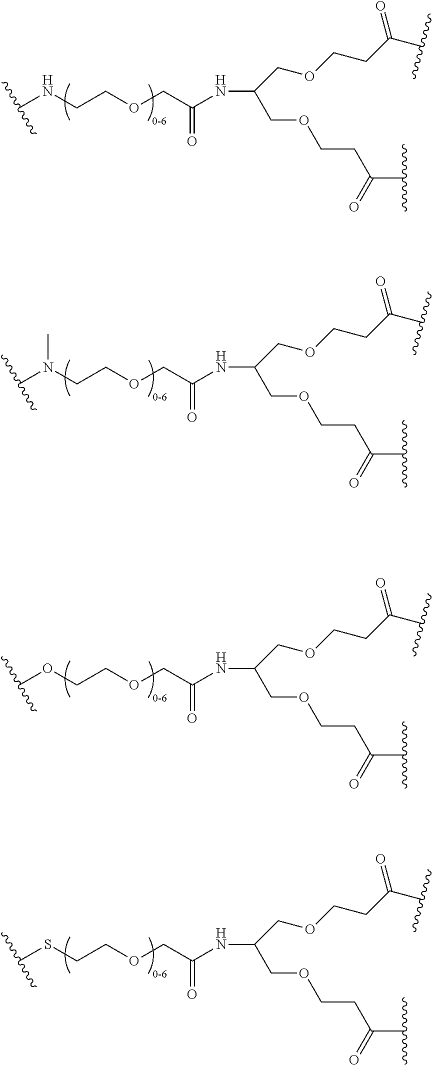

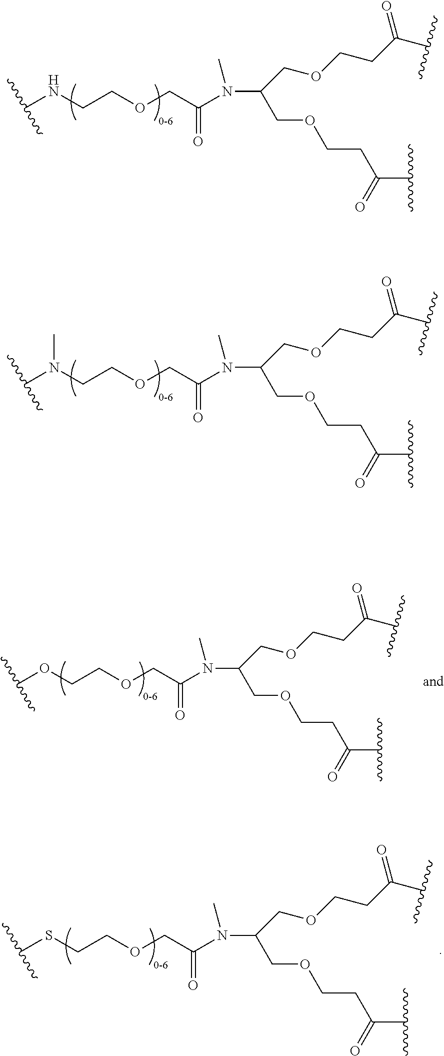

In certain embodiments Linker^{C} is selected from:

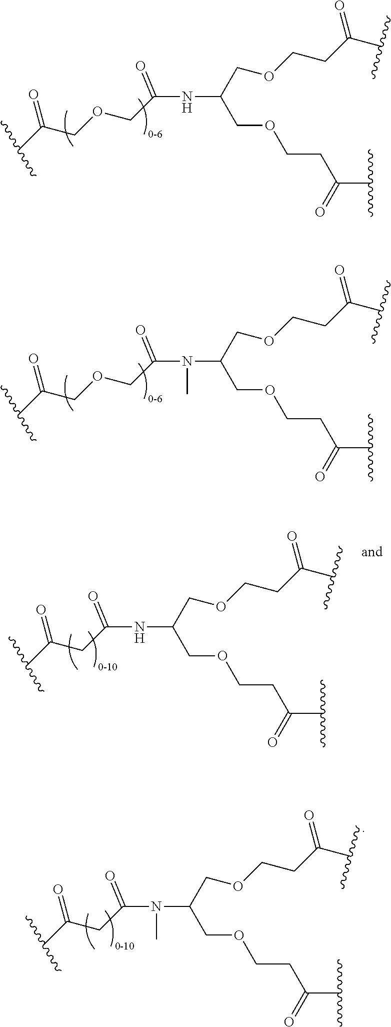

In certain embodiments Linker^{C} is selected from:

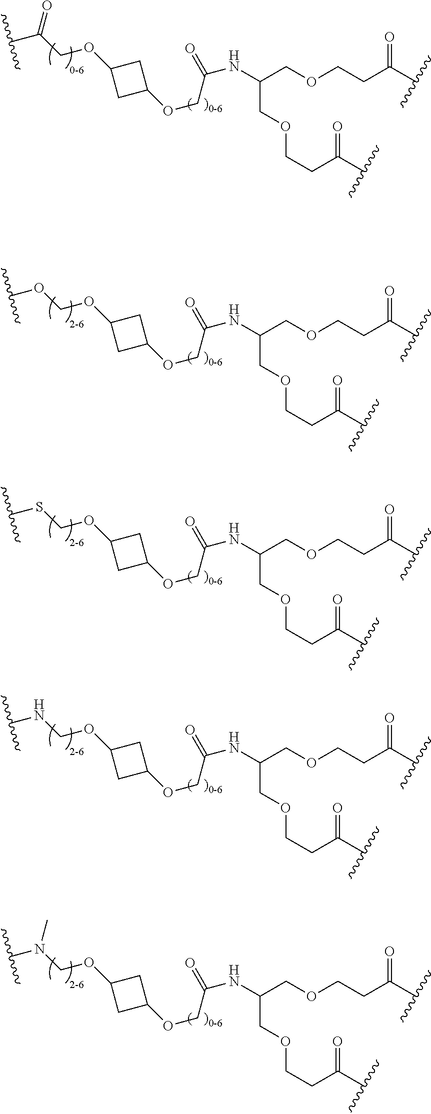

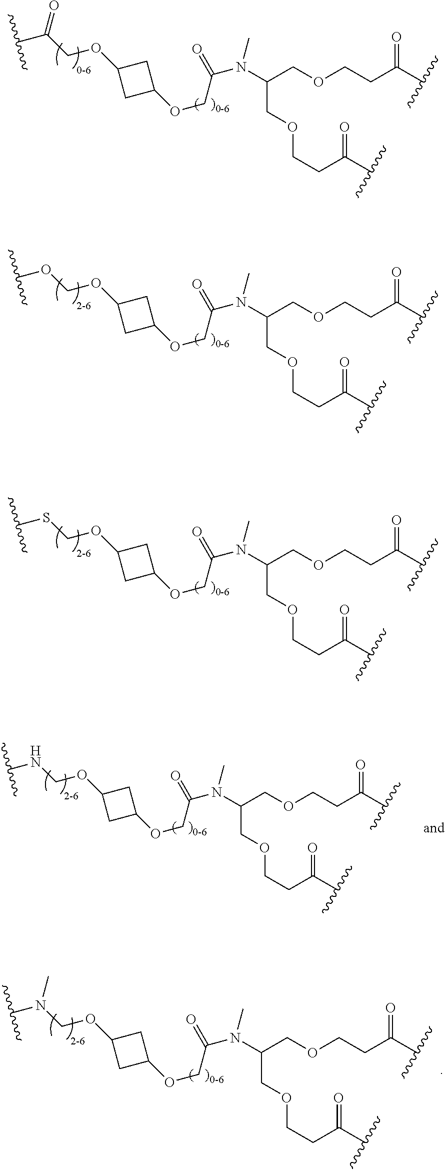

In certain embodiments Linker^{C} is selected from:

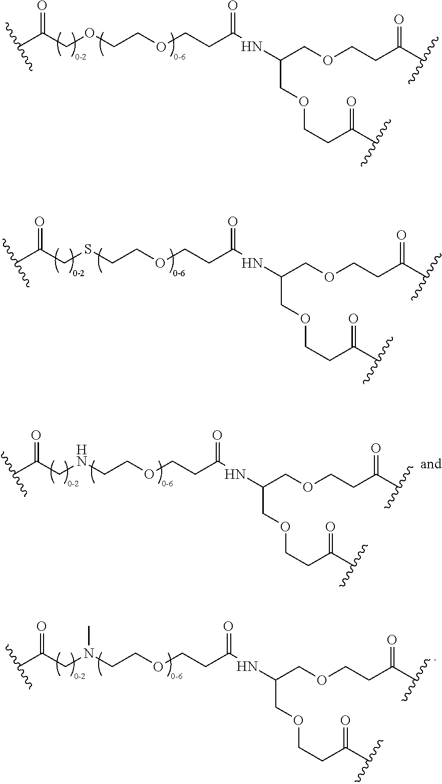

In certain embodiments Linker^{C} is selected from

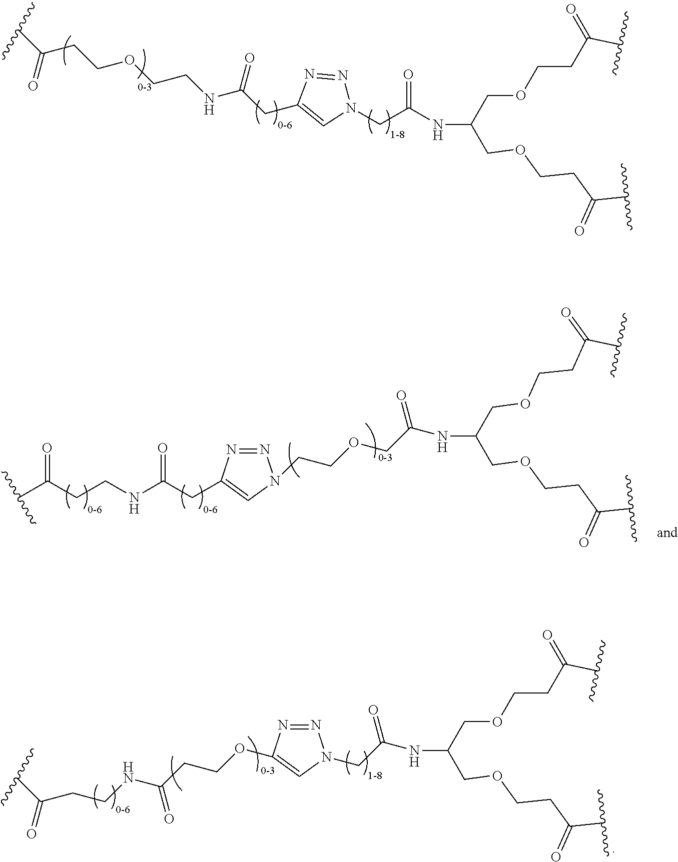

In certain embodiments Linker^{C} is selected from:

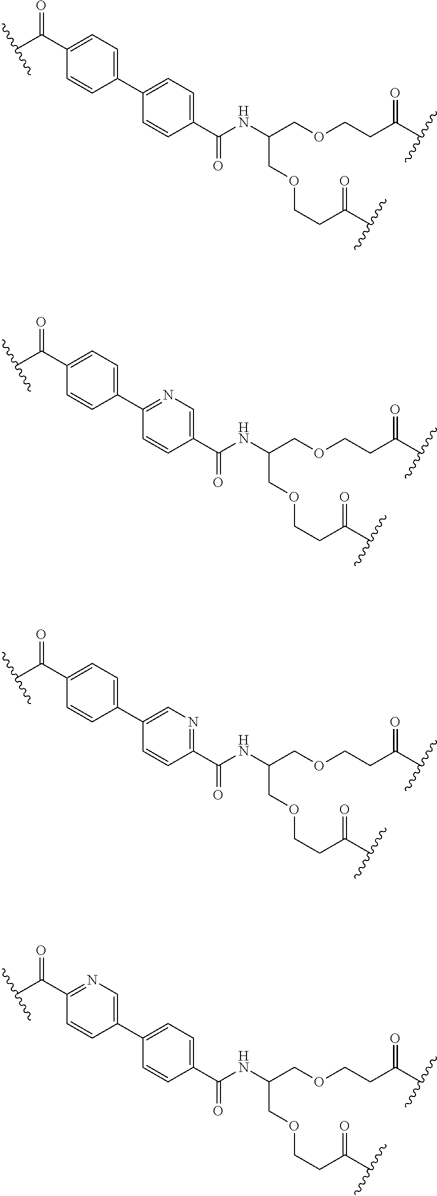 [SMILES: *C(=O)CCOCC(COCCC(*)=O)NC(=O)c1ccc(-c2ccc(C(*)=O)cc2)cc1 ; *C(=O)CCOCC(COCCC(*)=O)NC(=O)c1ccc(-c2ccc(C(*)=O)cc2)nc1 ; *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1]

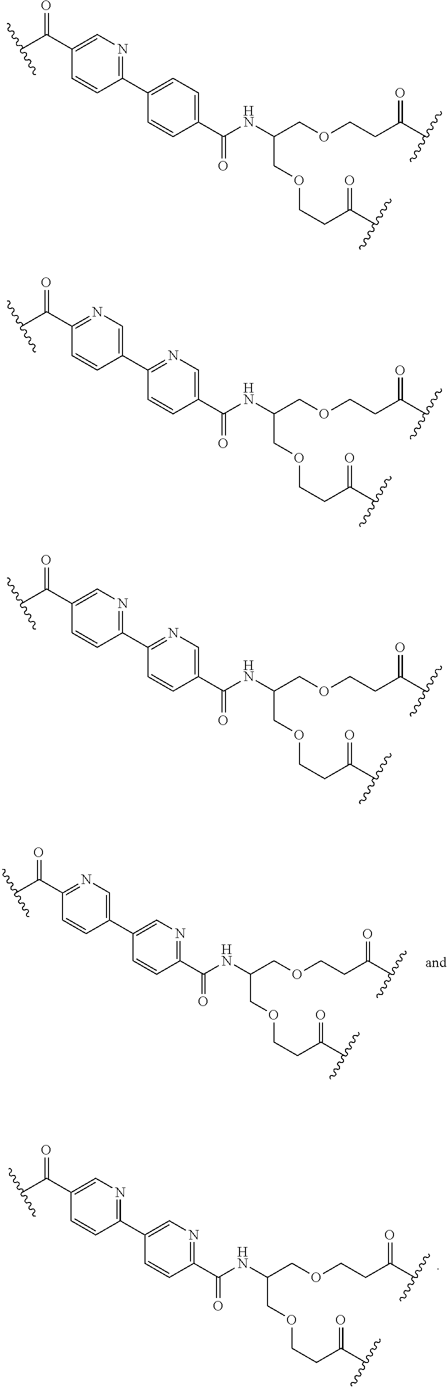 [SMILES: *C(=O)CCOCC(COCCC(*)=O)NC(=O)c1ccc(-c2ccc(C(*)=O)cn2)cc1 ; *C(=O)CCOCC(COCCC(*)=O)NC(=O)c1ccc(-c2ccc(C(*)=O)nc2)nc1 ; *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments Linker^{D} is selected from:

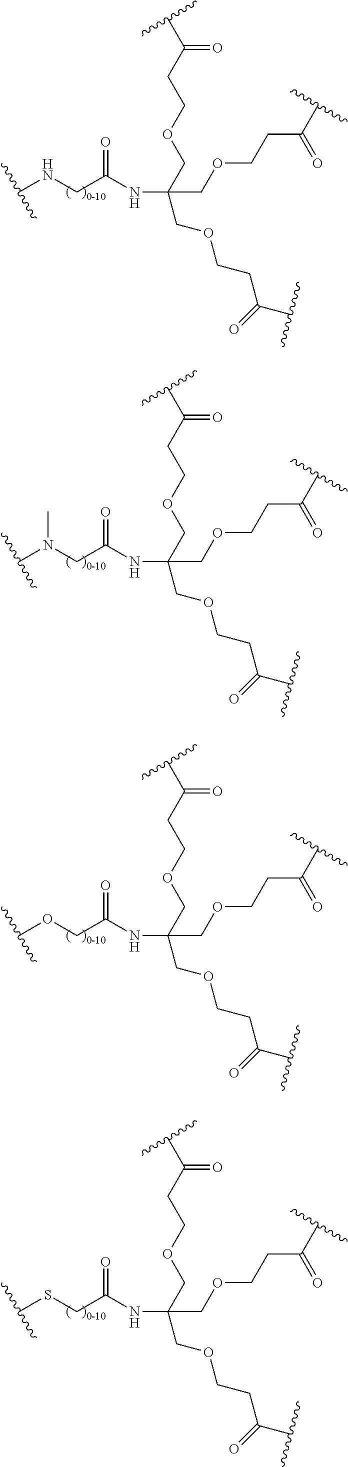

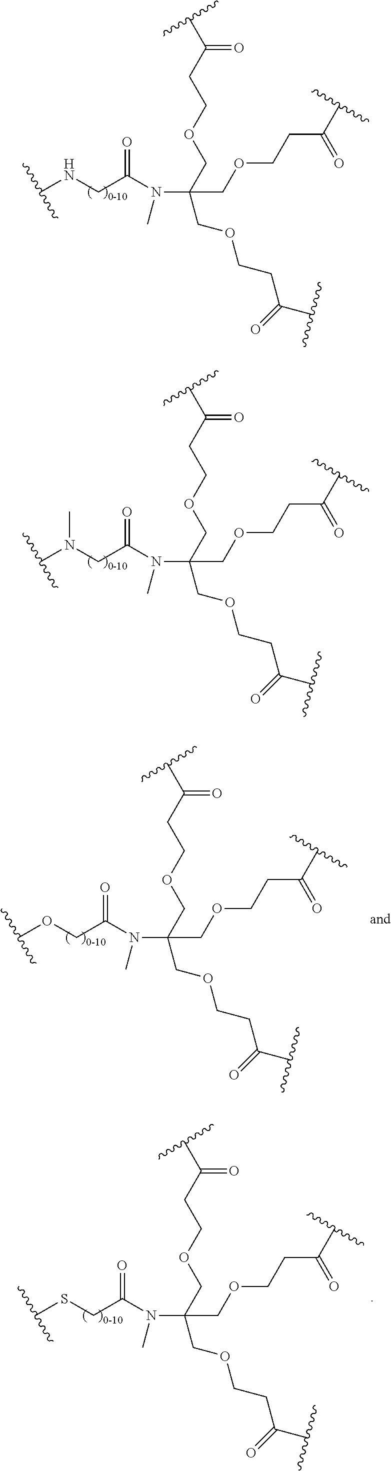

In certain embodiments Linker^{D} is selected from:

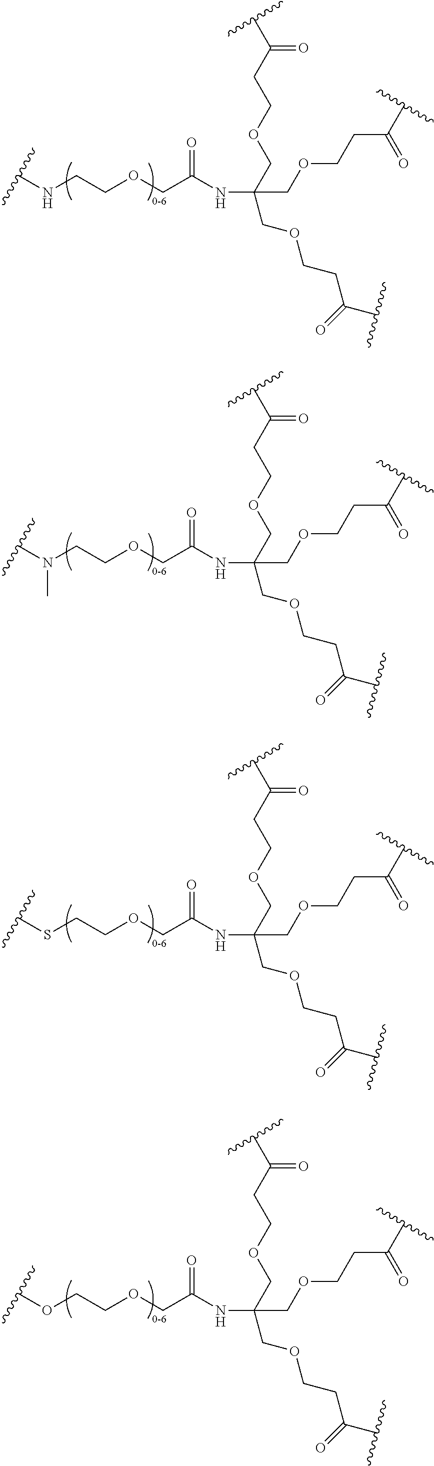

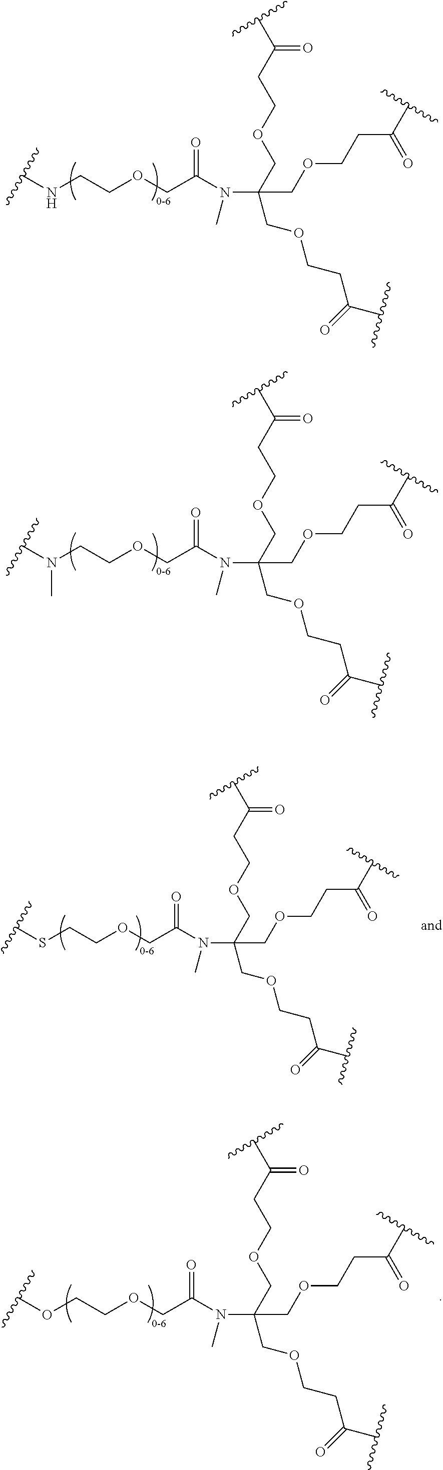

In certain embodiments Linker^{D} is selected from:

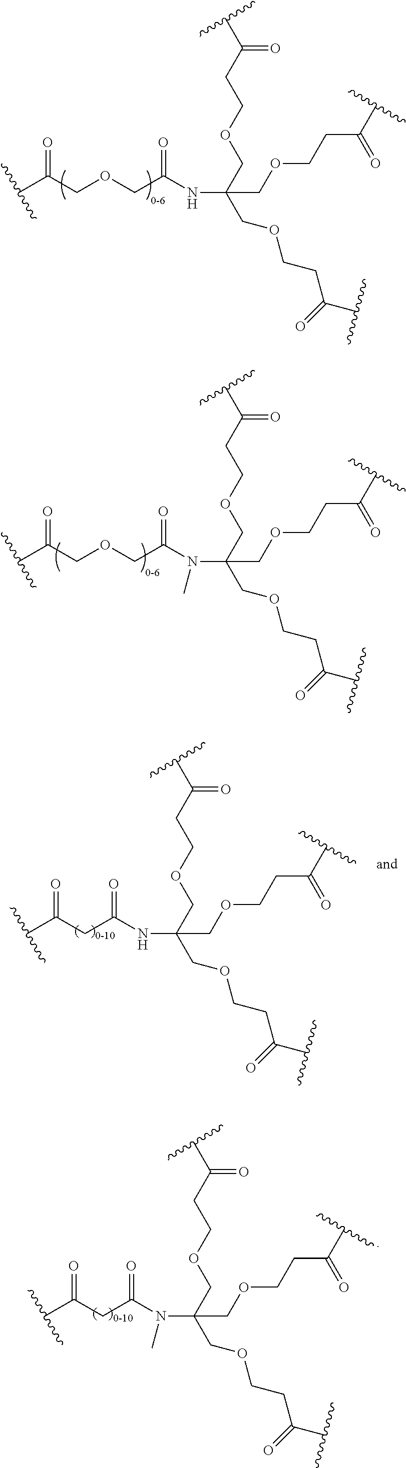

In certain embodiments Linker^{D} is selected from:

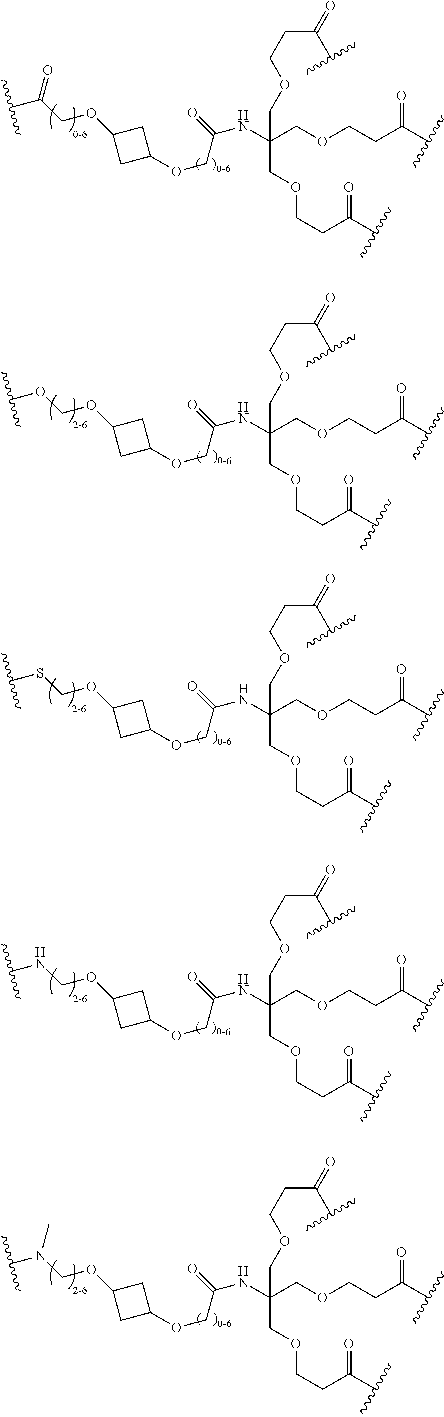

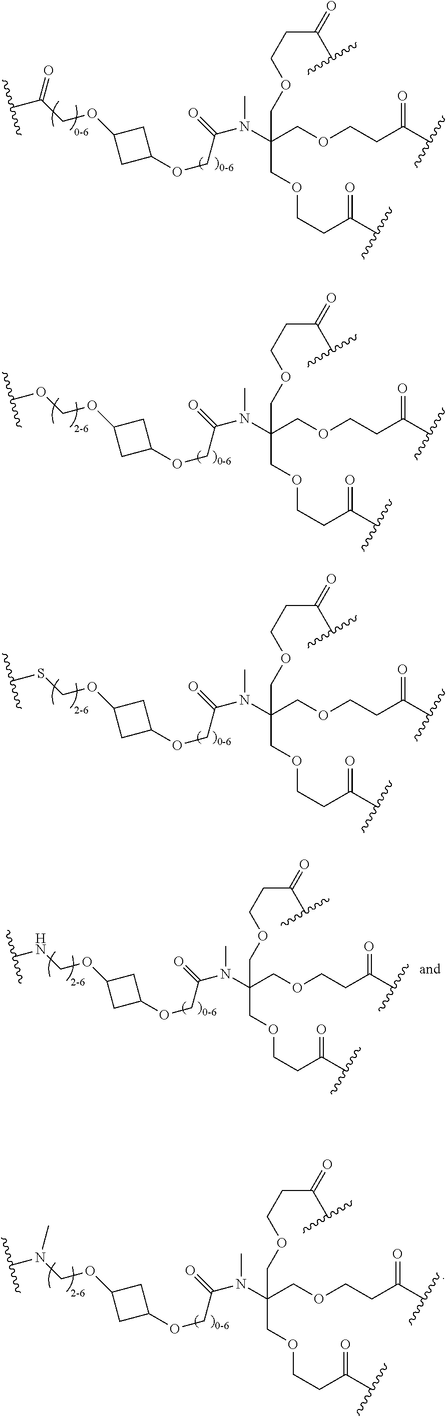

In certain embodiments Linker^{D} is selected from:

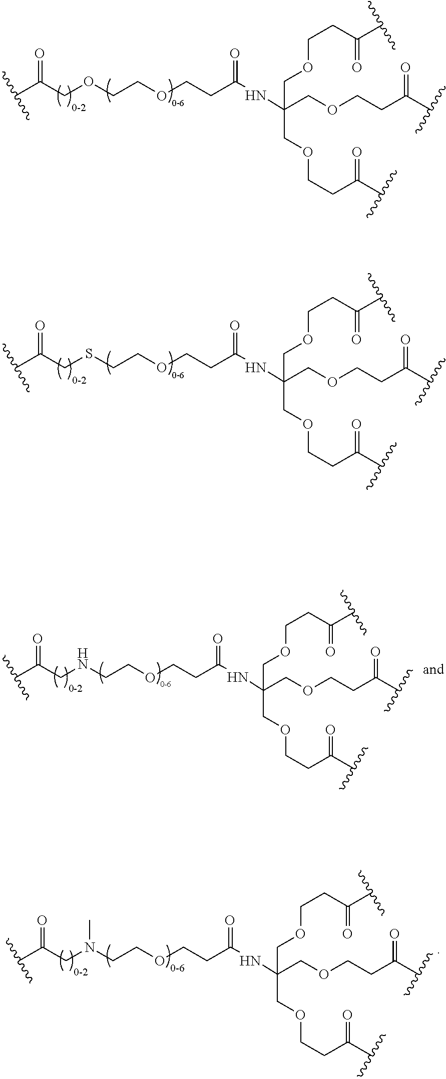

In certain embodiments Linker^{D} is selected from:

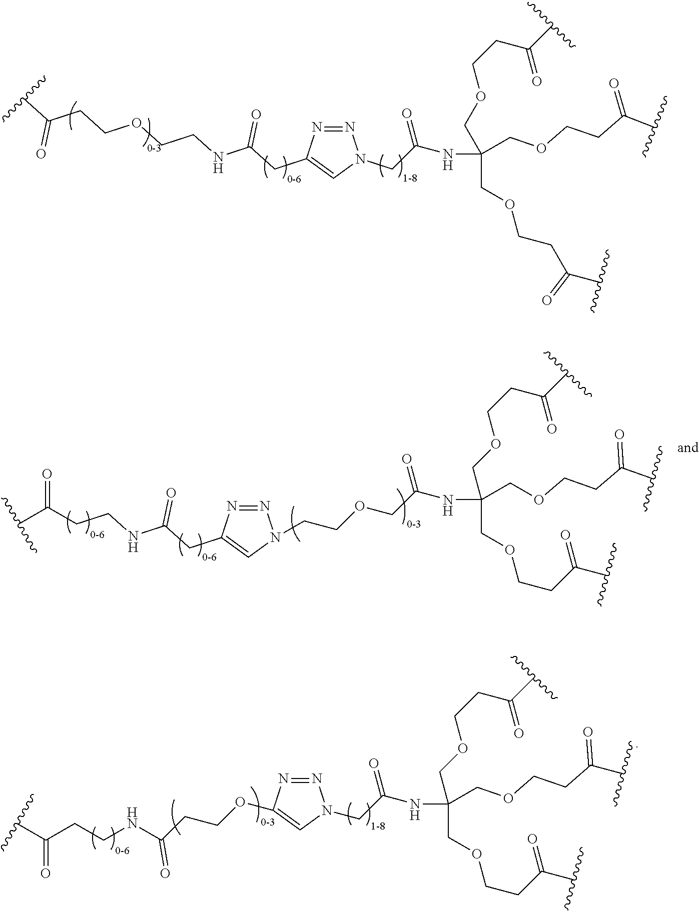

In certain embodiments Linker^{D} is selected from:

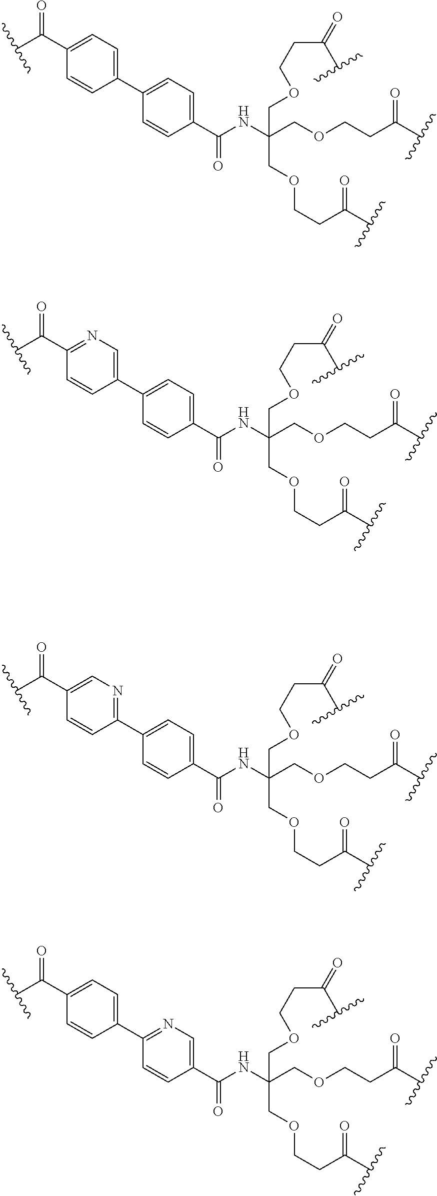 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1]

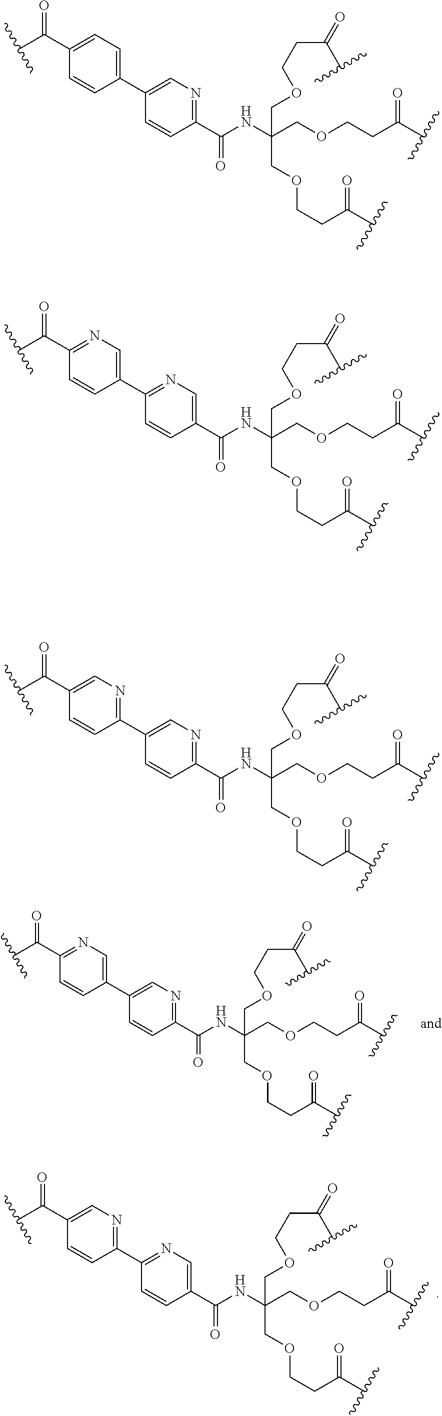 [SMILES: *C(=O)CCOCC(COCCC(*)=O)(COCCC(*)=O)NC(=O)c1ccc(-c2ccc(C(*)=O)cc2)cn1 ; *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, the Linker^{A} is selected from

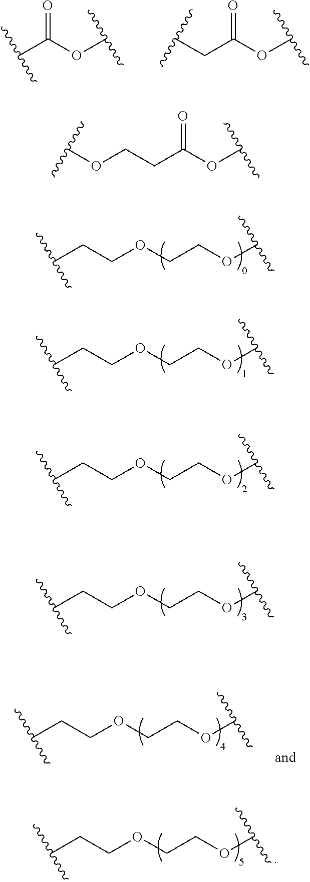

In certain embodiments, the Linker^{A} is selected from

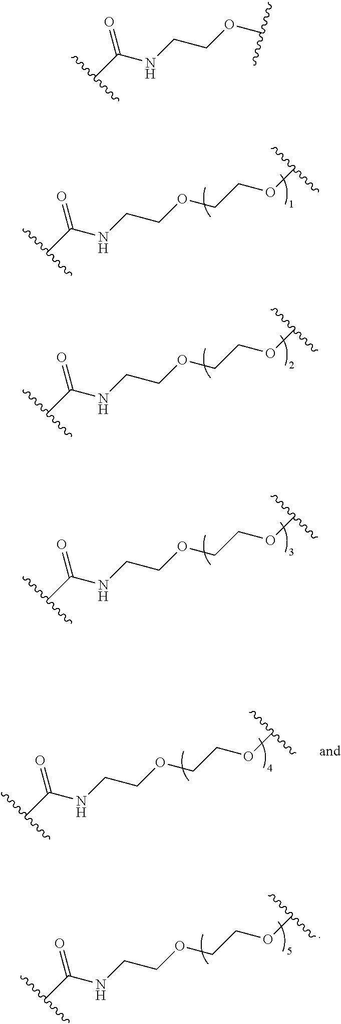

In certain embodiments, the Linker^{A} is selected from

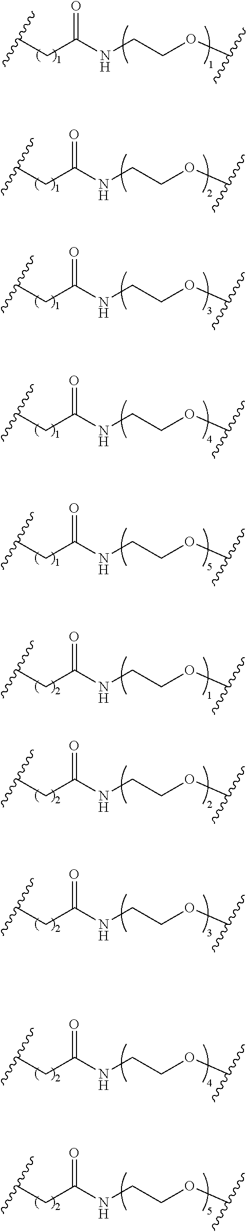

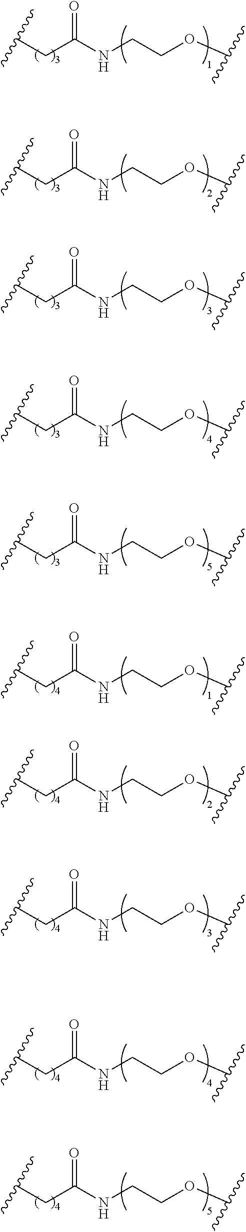

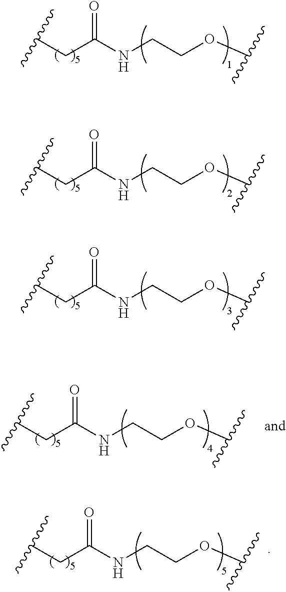

In certain embodiments, the Linker^{A} is selected from

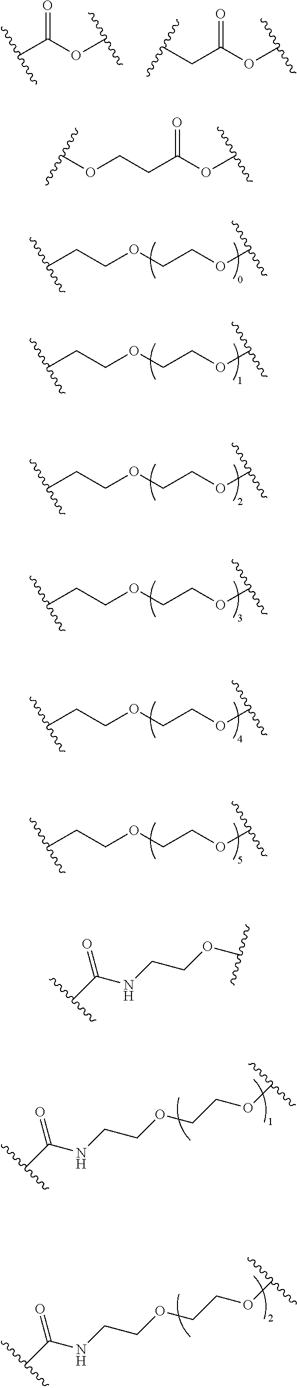

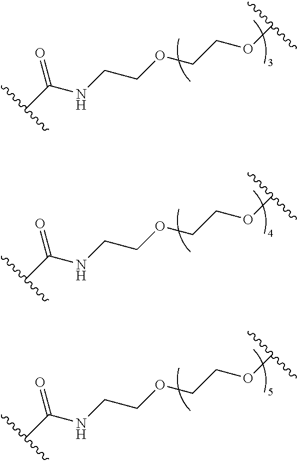

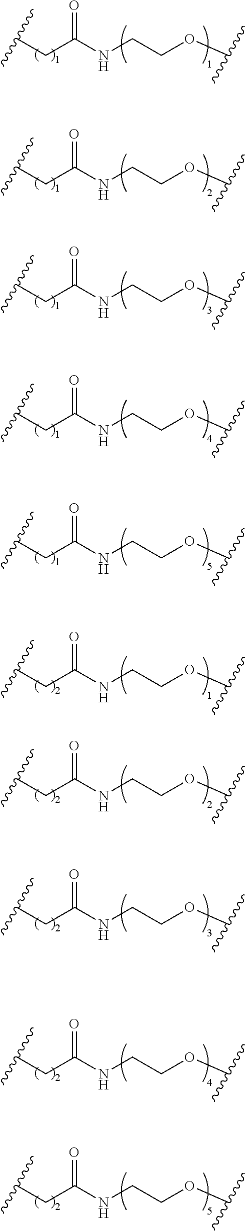

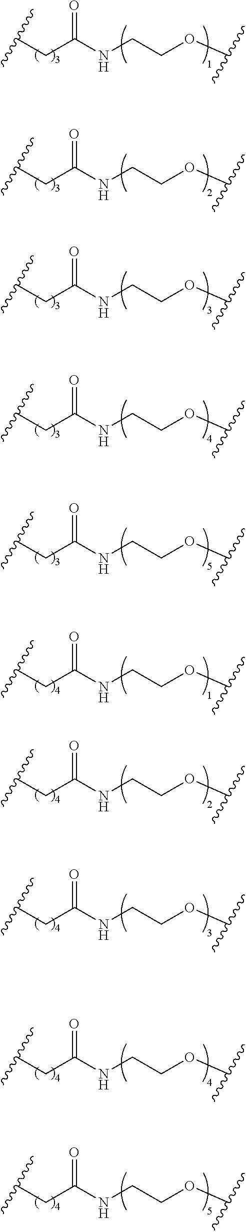

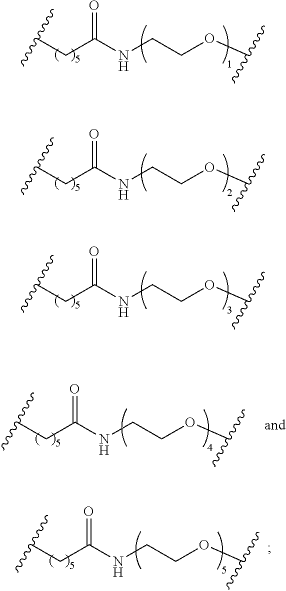

wherein each is optionally substituted with 1, 2, 3, or 4 substituents substituent selected from R²¹.

In certain embodiments Linker^{A} is selected from:

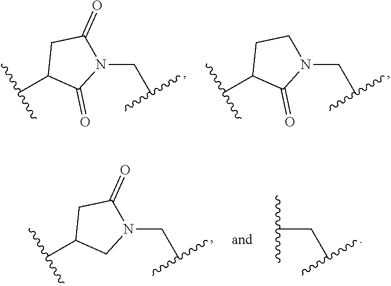

In certain embodiments Linker^{A} is selected from:

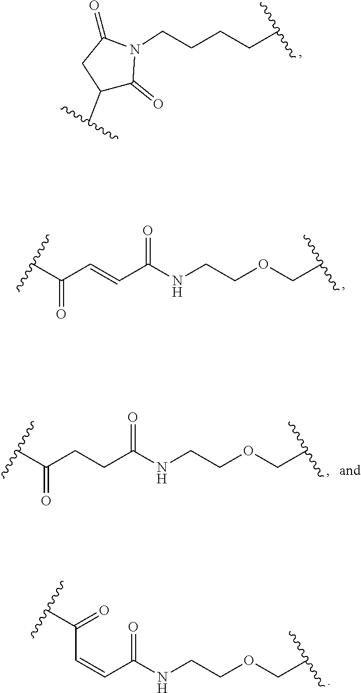 [SMILES: *CCCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments Linker^{A} is selected from:

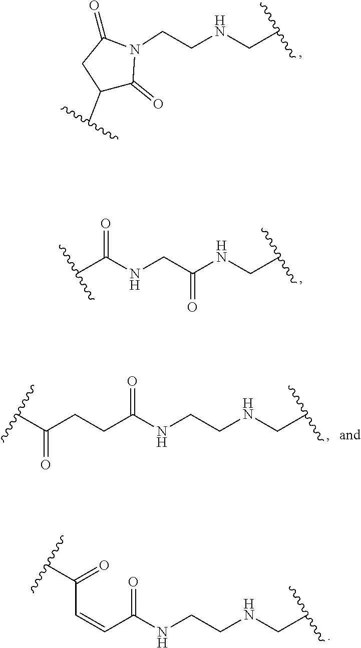 [SMILES: *CNCCN1C(=O)CC(*)C1=O ; *CNC(=O)CNC(*)=O ; *CNCCNC(=O)CCC(*)=O ; *CNCCNC(=O)/C=C\C(*)=O]

In certain embodiments Linker^{A} is selected from:

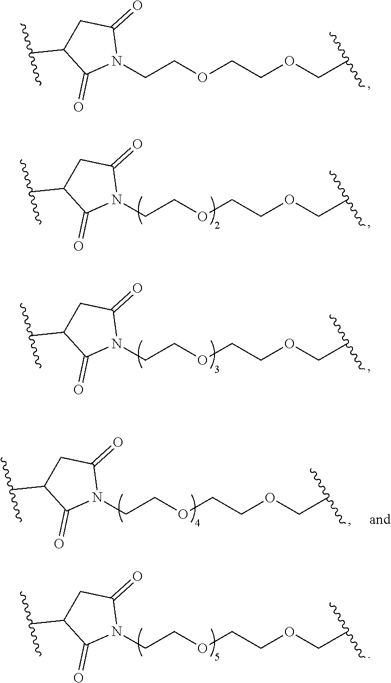

In certain embodiments Linker^{A} is selected from:

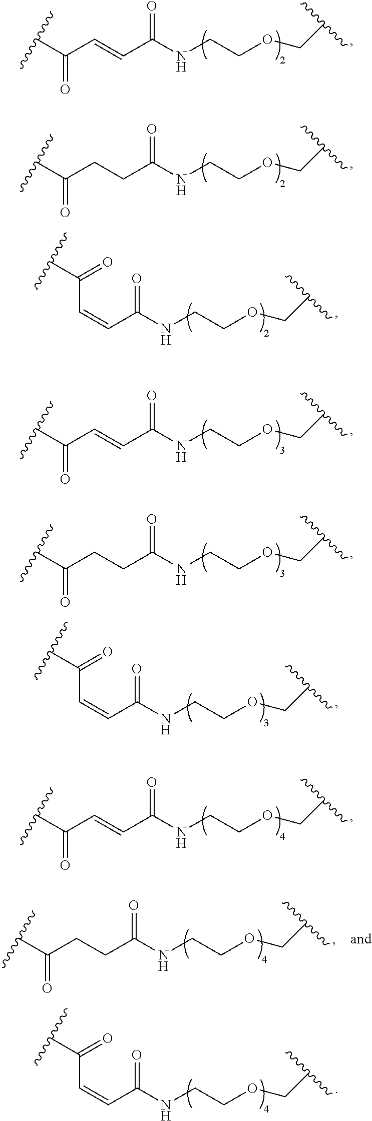

In certain embodiments Linker^{A} is selected from:

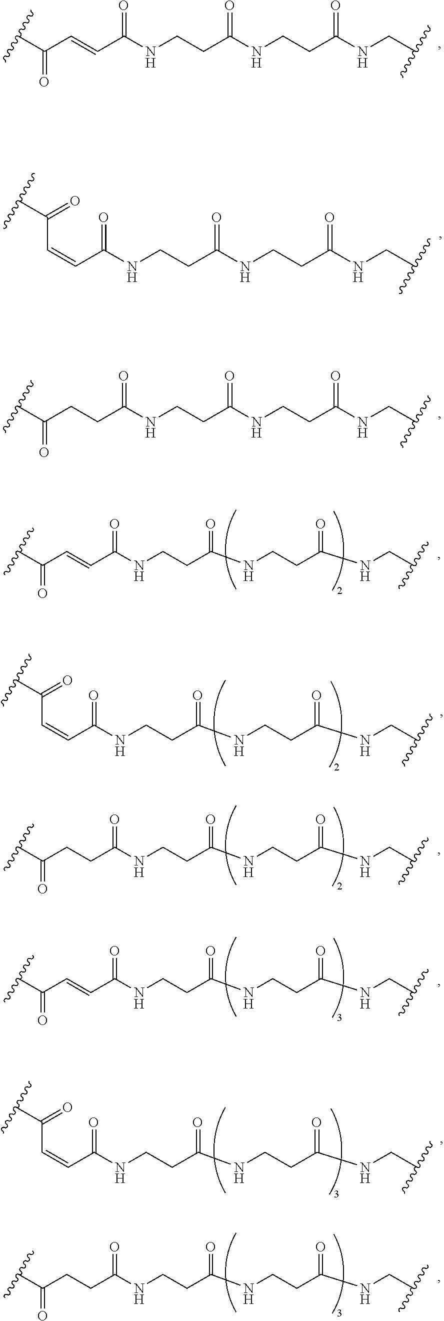

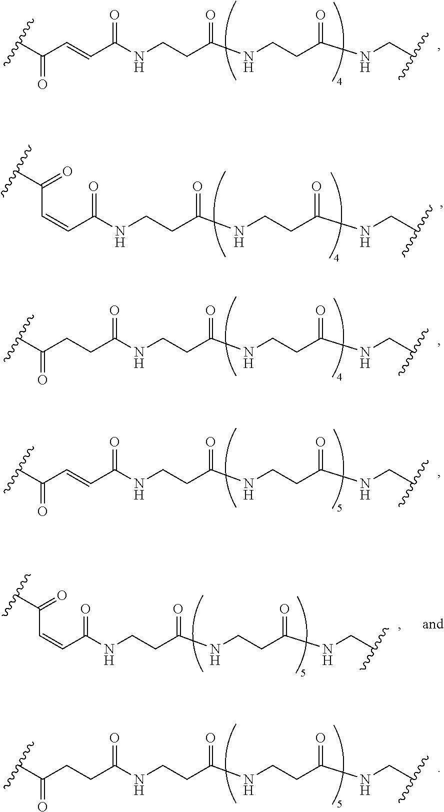

In certain embodiments Linker^{A} is selected from:

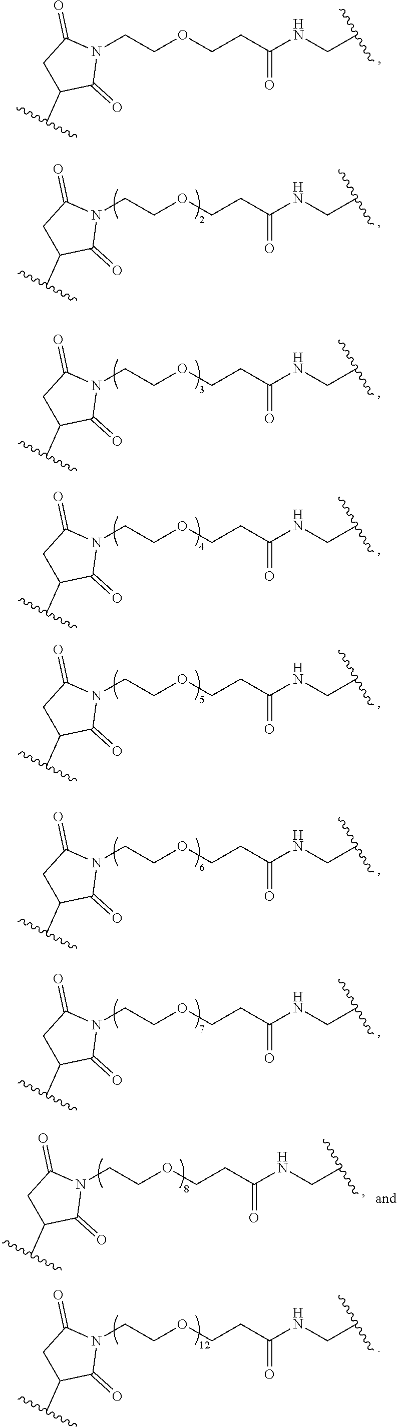

In certain embodiments Linker^{A} is selected from:

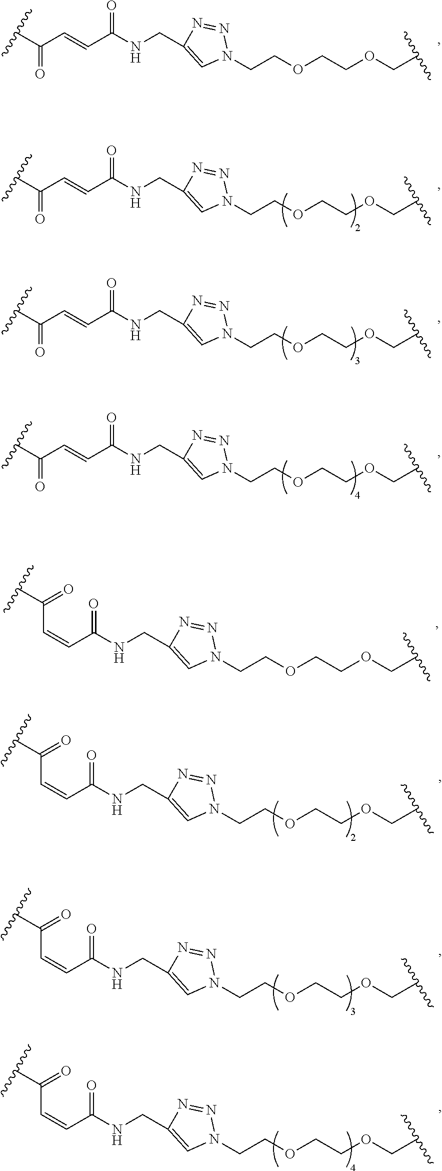

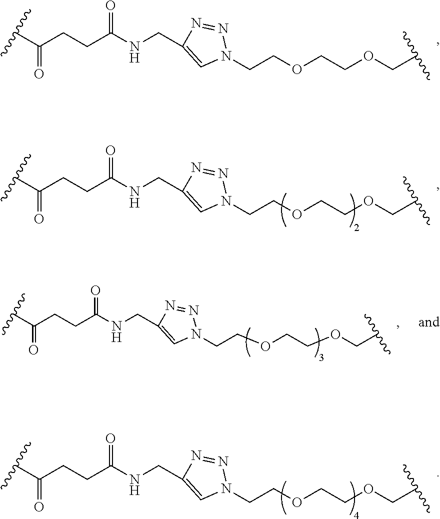

In certain embodiments Linker^{A} is selected from:

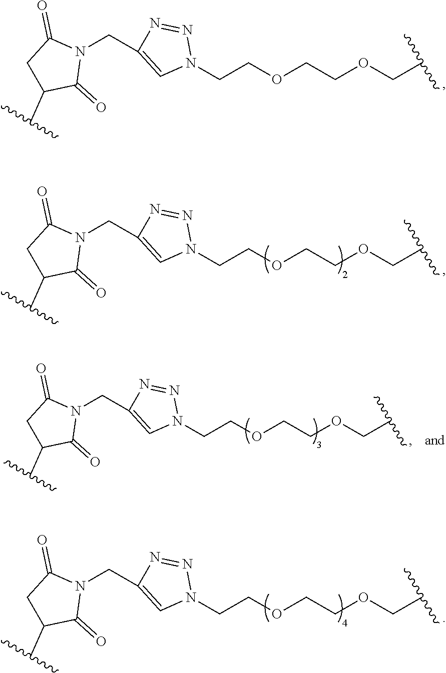

In certain embodiments Linker^{A} is selected from:

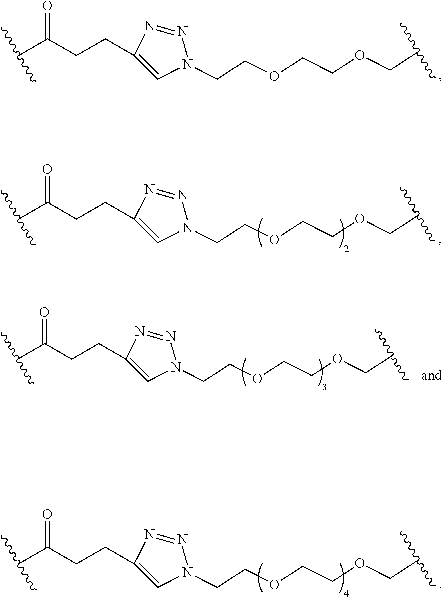

In certain embodiments Linker^{A} is selected from:

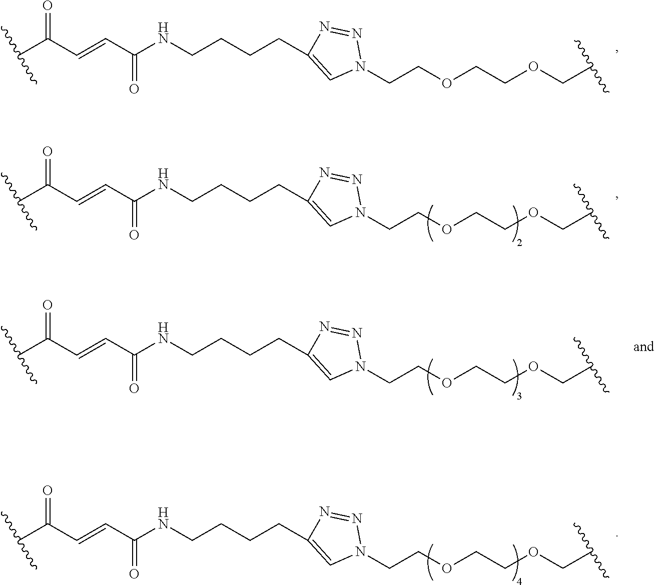

In certain embodiments Linker^{A} is selected from:

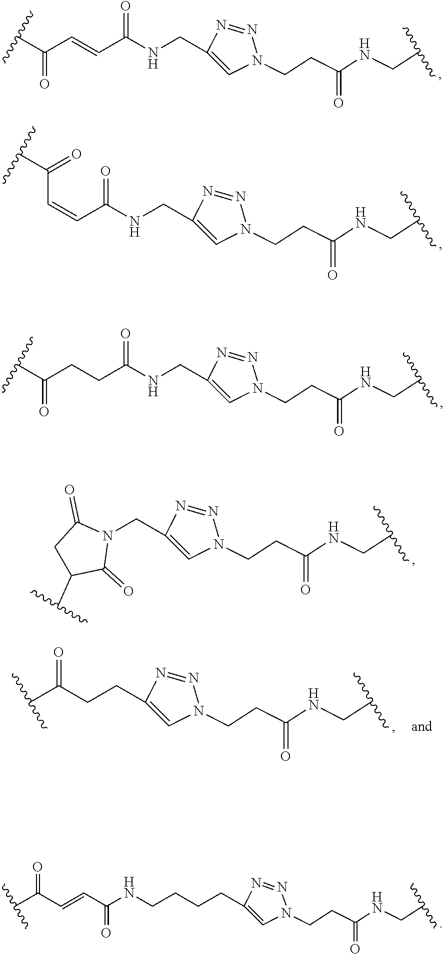 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments Linker^{A} is selected from:

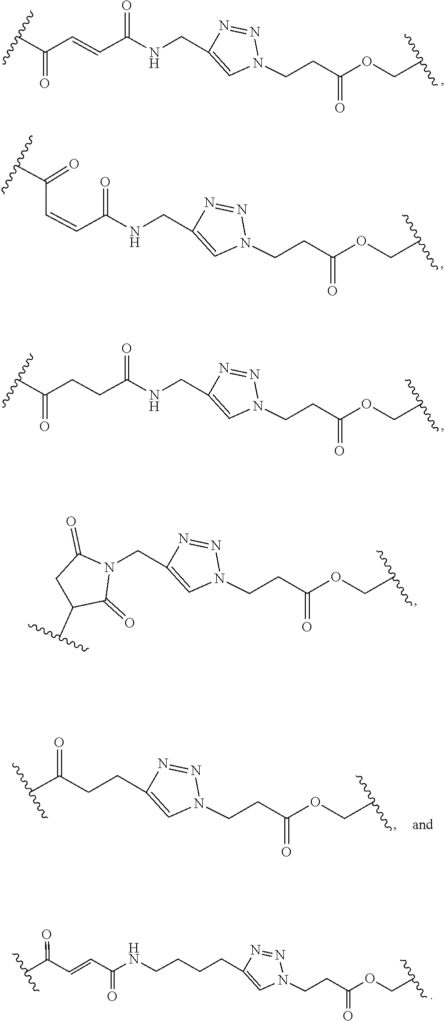 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments, the Linker^{B} is selected from

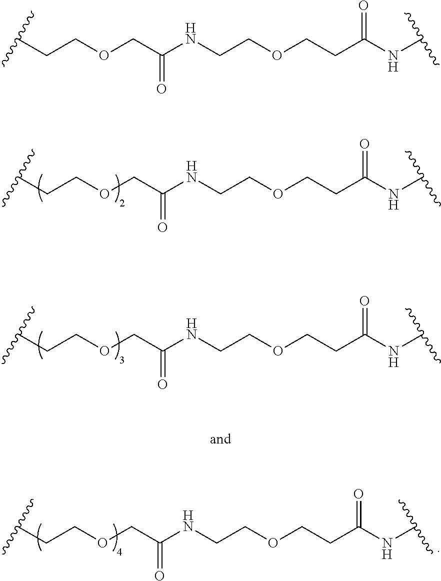

In certain embodiments, the Linker^{B} is selected from

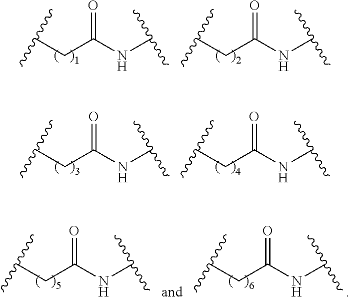

In certain embodiments, the Linker^{B} is selected from

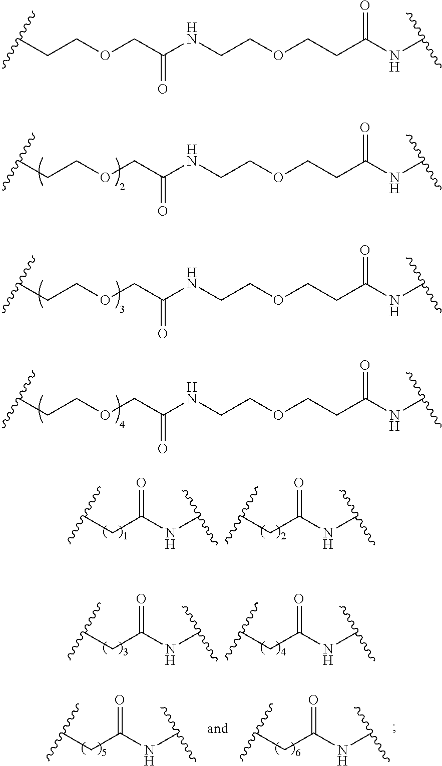

wherein each is optionally substituted with 1, 2, 3, or 4 substituents substituent selected from R²¹.

In certain embodiments Linker^{B} is selected from:

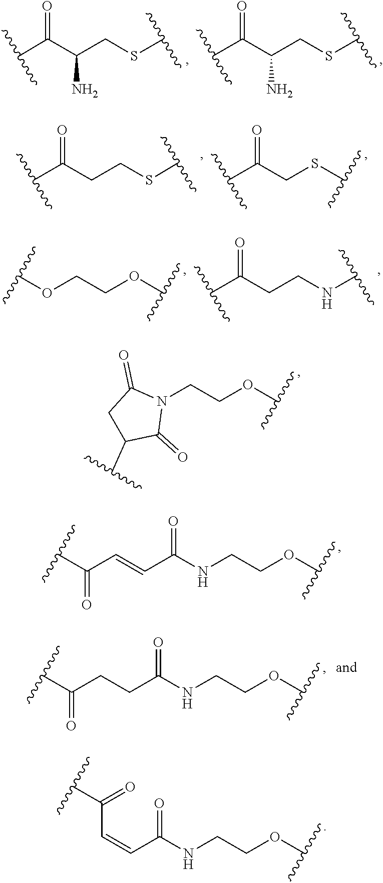

In certain embodiments Linker^{B} is selected from:

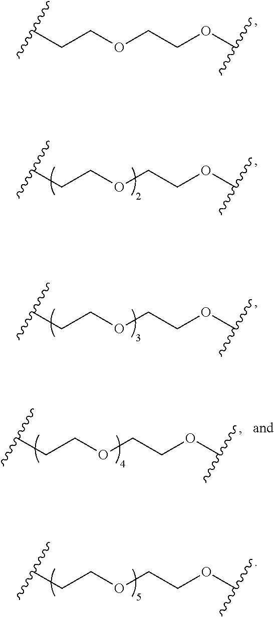

In certain embodiments Linker^{B} is selected from:

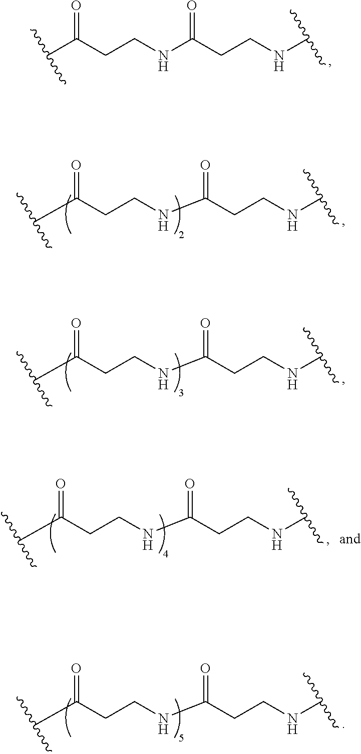

In certain embodiments Linker^{B} is selected from:

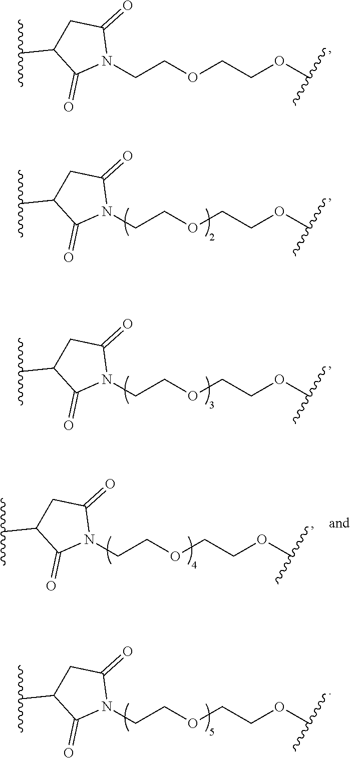

In certain embodiments Linker^{B} is selected from:

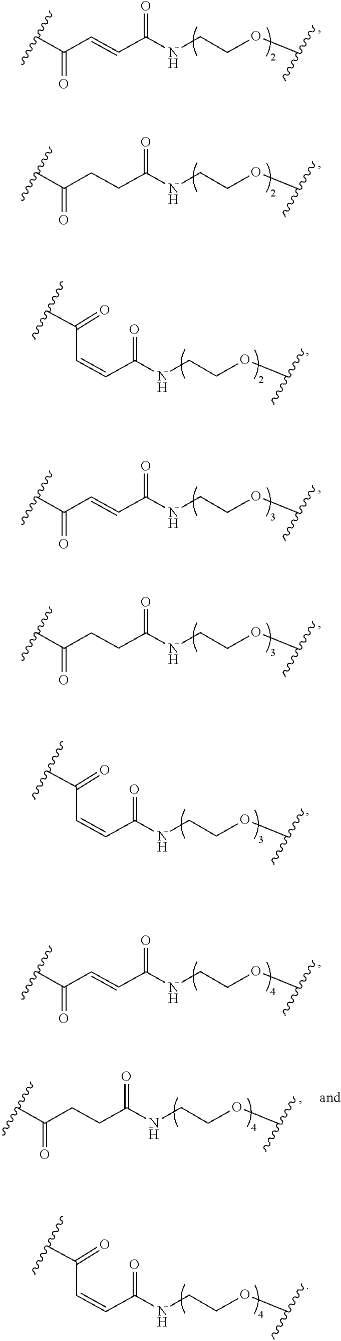

In certain embodiments Linker^{B} is selected from:

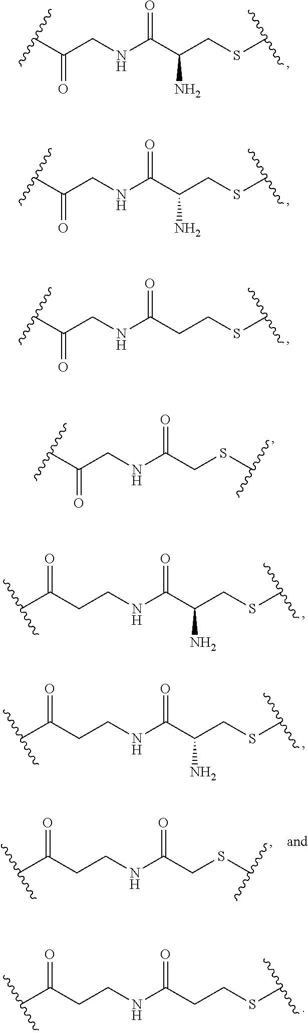

In certain embodiments Linker^{B} is selected from:

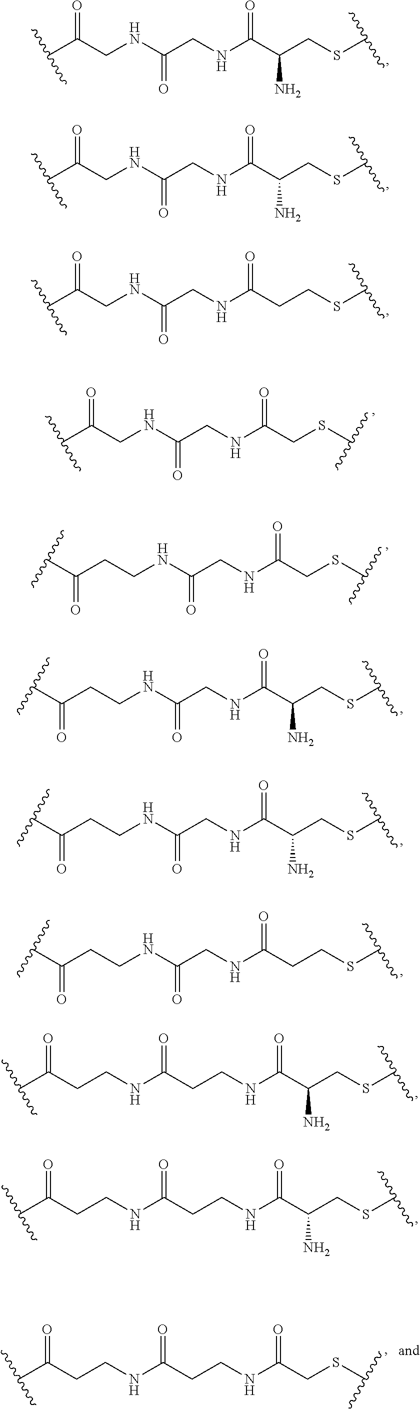

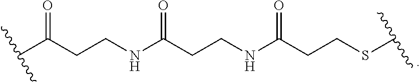 [SMILES: *SCCC(=O)NCCC(=O)NCCC(*)=O]

In certain embodiments Linker^{B} is selected from:

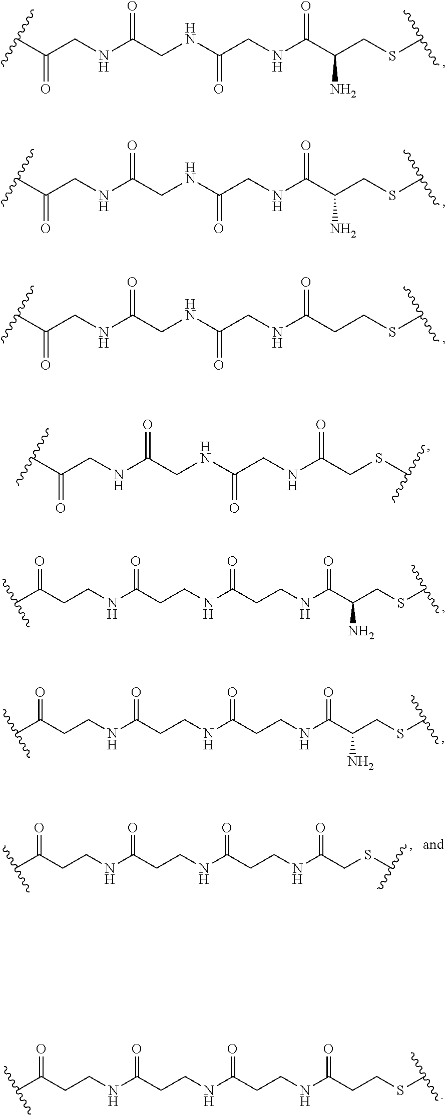

In certain embodiments Linker^{B}-Linker^{A} is selected from:

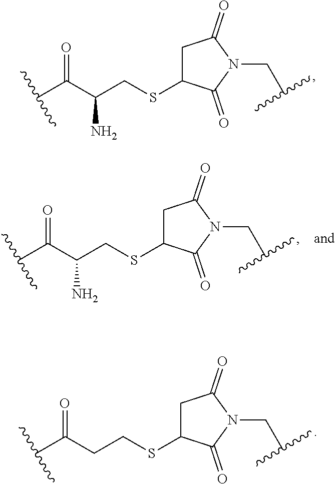 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments Linker^{B}-Linker^{A} is selected from:

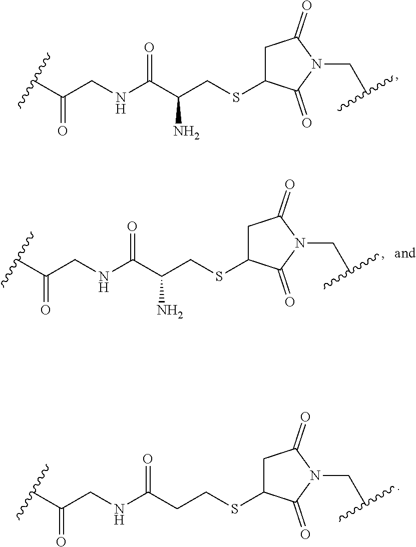 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, Linker^{C} is selected from

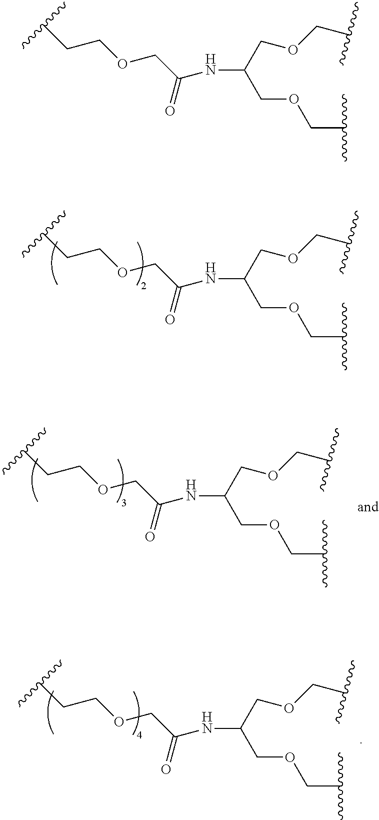

In certain embodiments, Linker^{C} is selected from

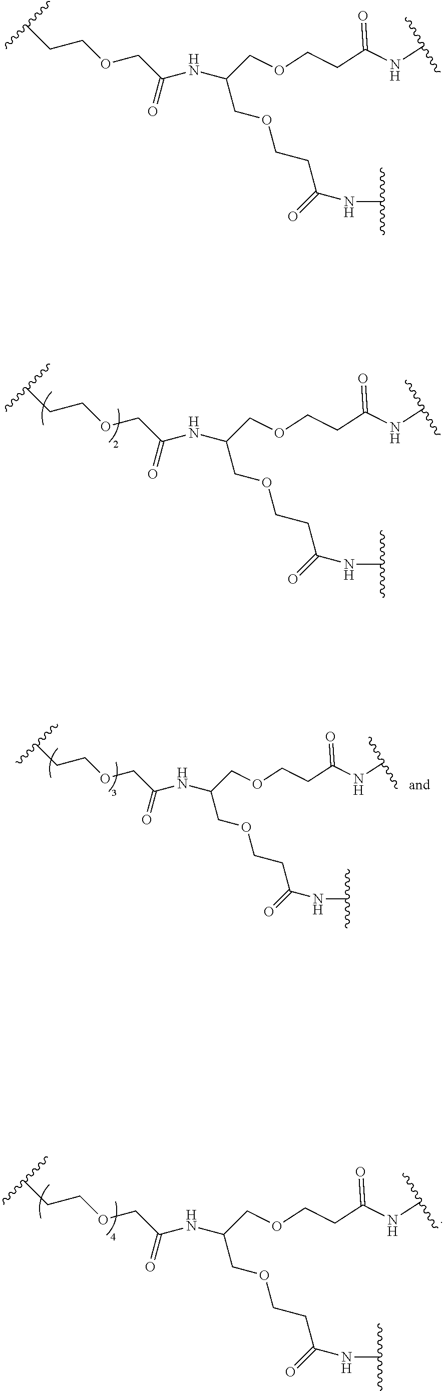

In certain embodiments, Linker^{C} is selected from

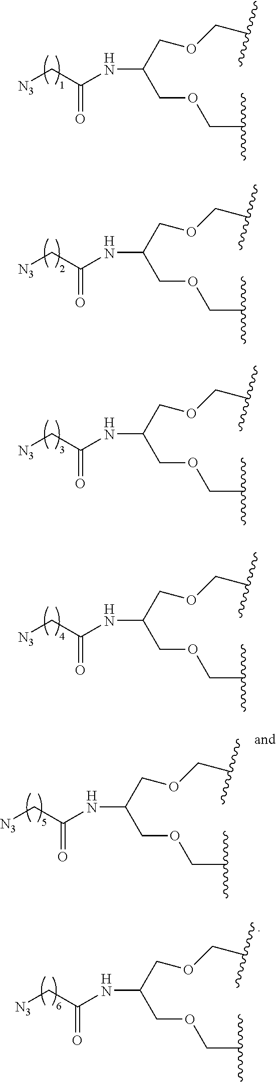

In certain embodiments Linker^{C} is selected from

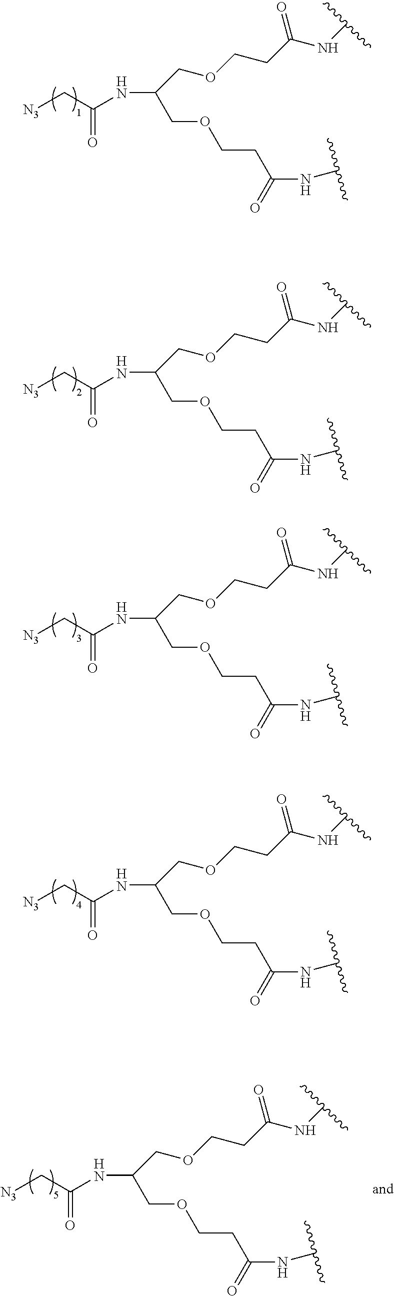

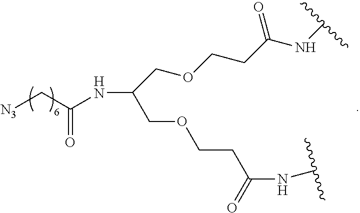

In certain embodiments, Linker^{C} is selected from

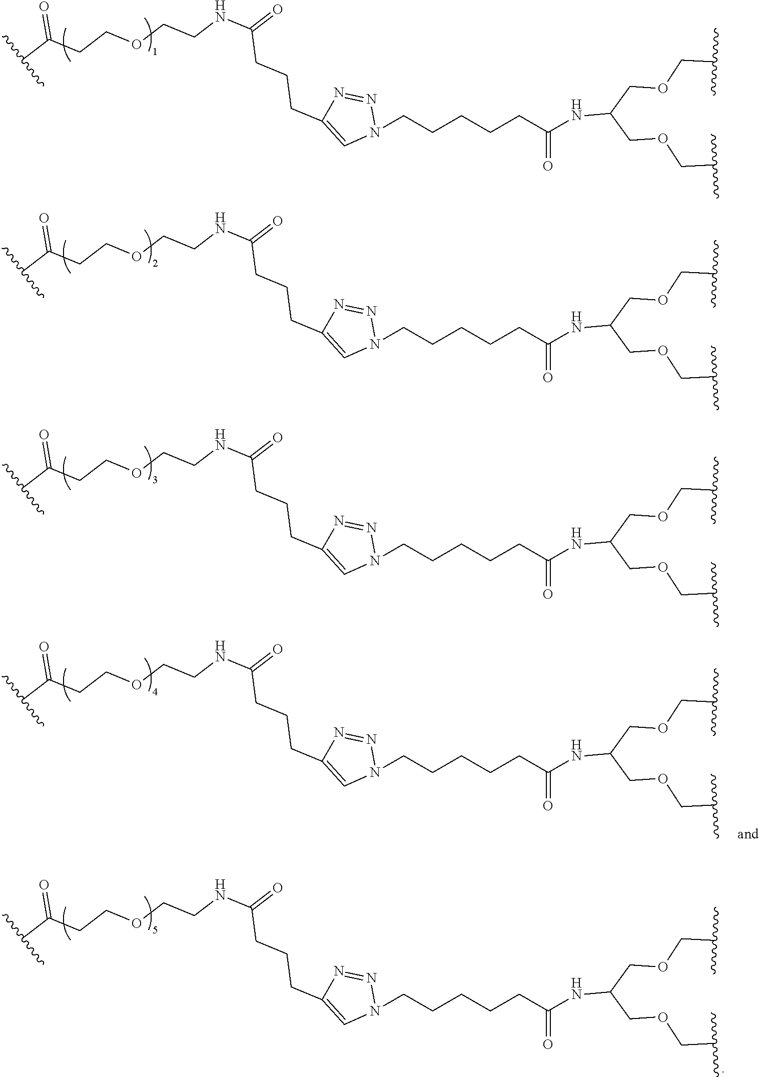

In certain embodiments, Linker^{C} is selected from

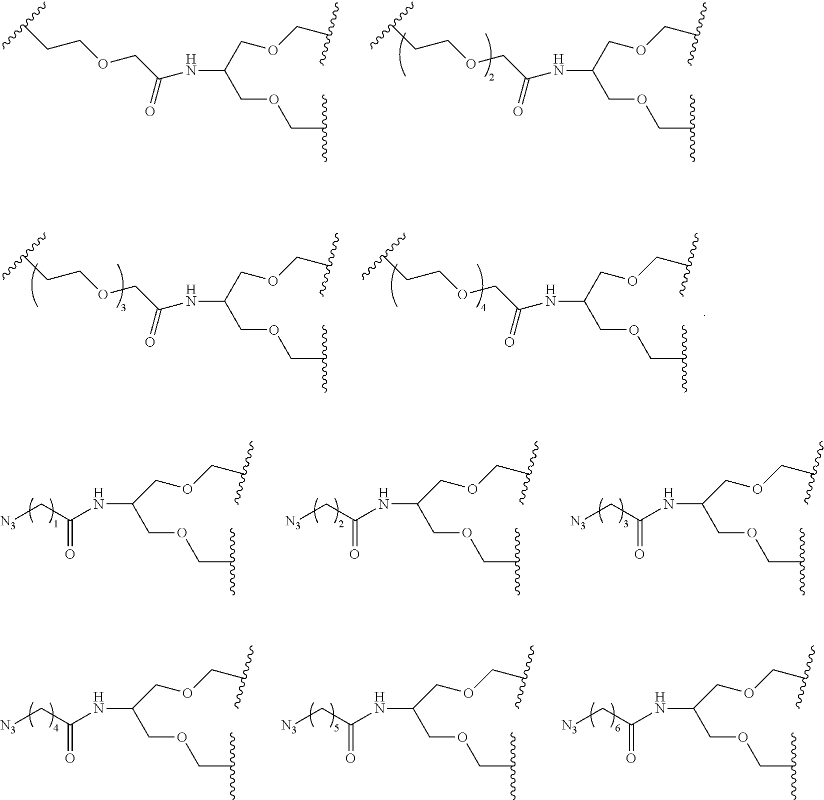

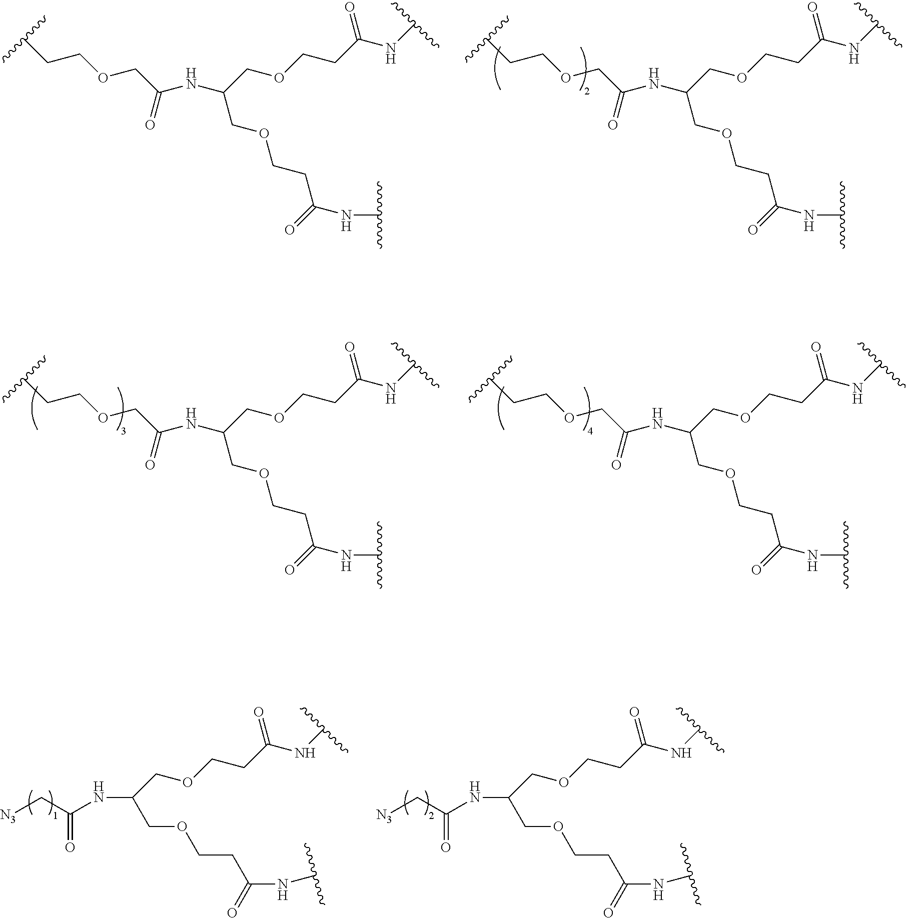

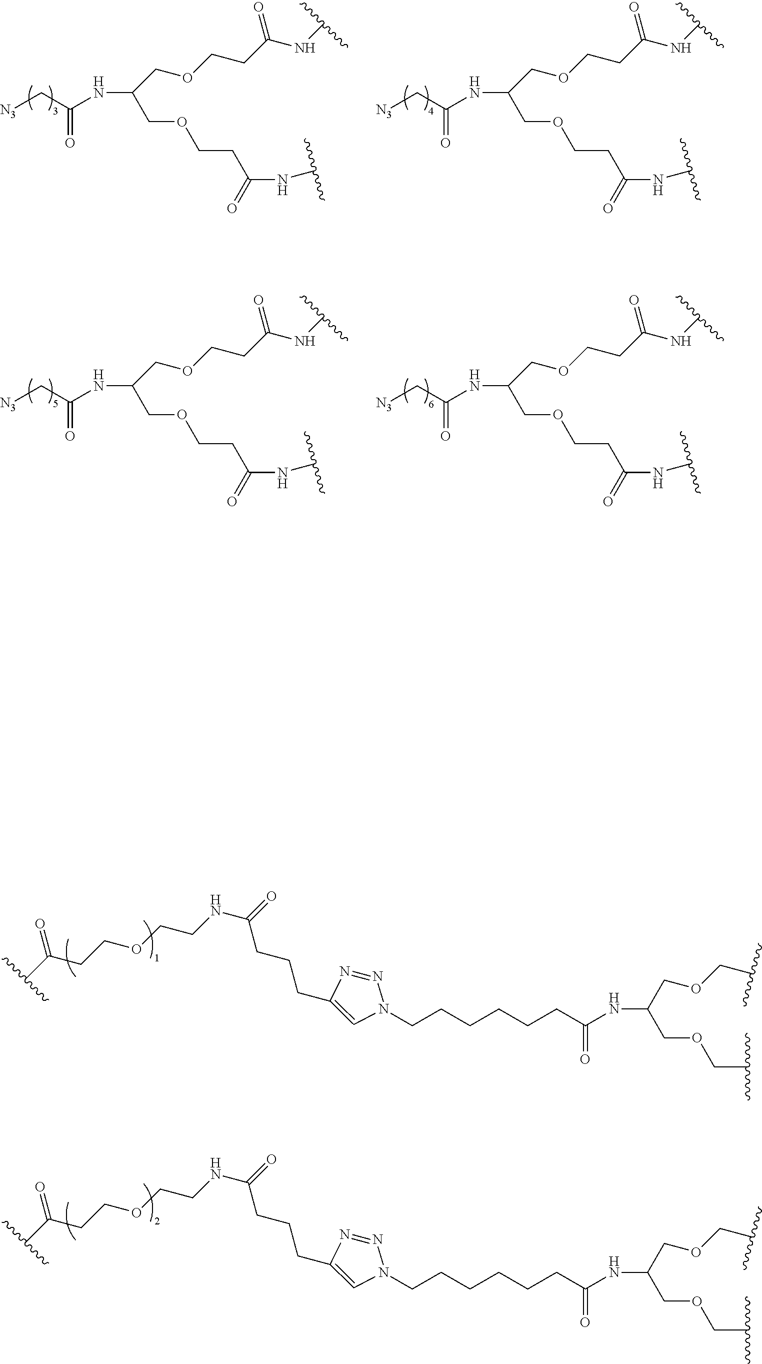

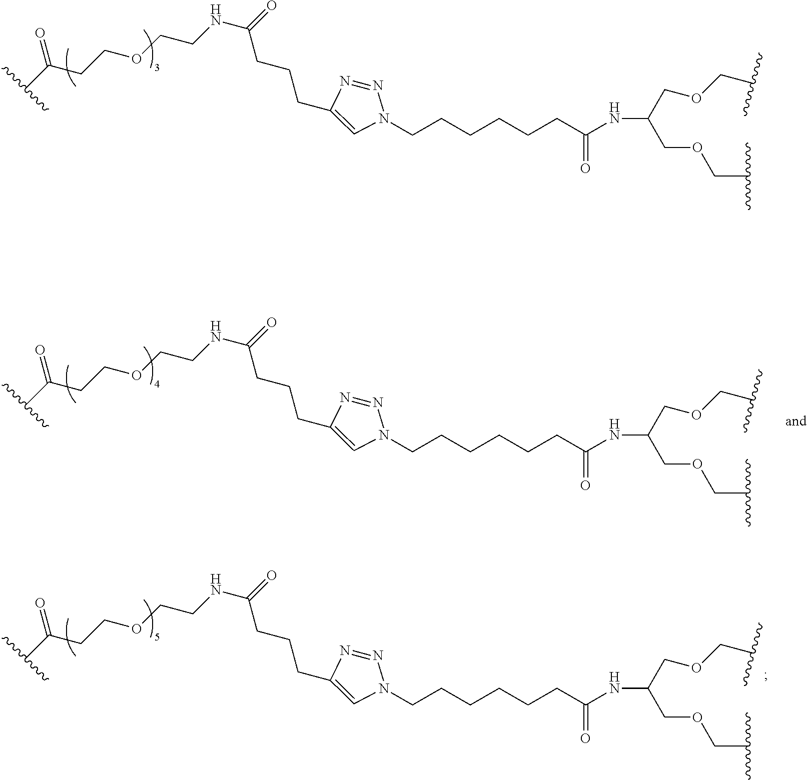

wherein each is optionally substituted with 1, 2, 3, or 4 substituents substituent selected from R²¹.

In certain embodiments Linker^{C} is selected from:

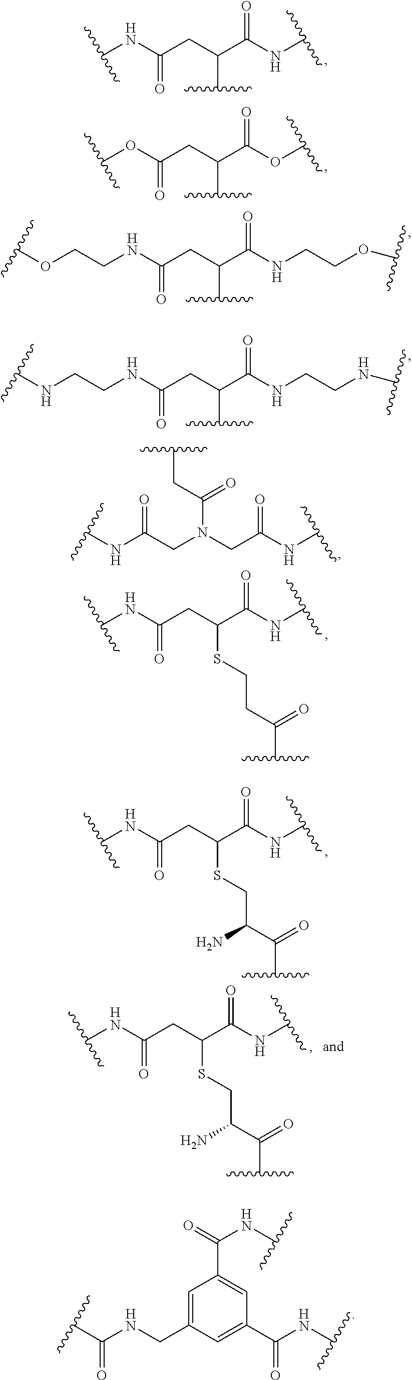

In certain embodiments Linker^{C} is selected from:

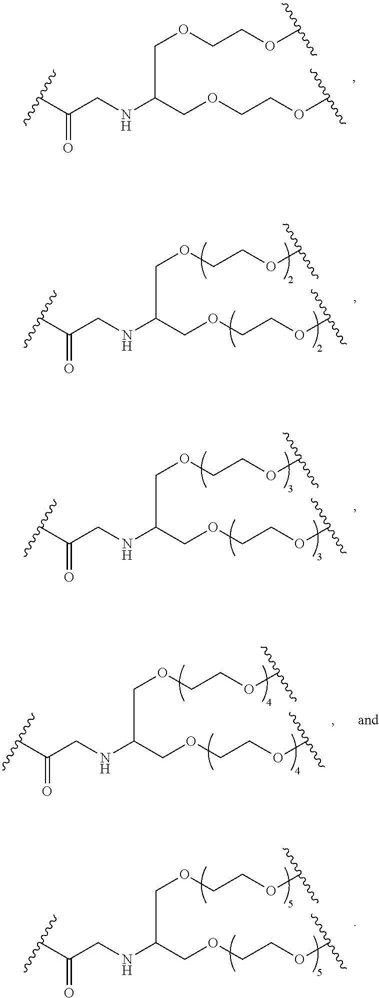

In certain embodiments Linker^{C} is selected from:

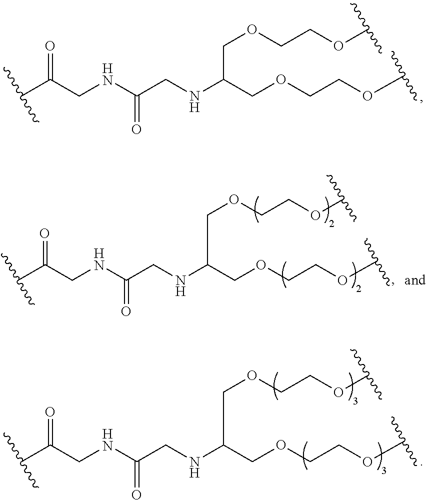

In certain embodiments Linker^{C} is selected from:

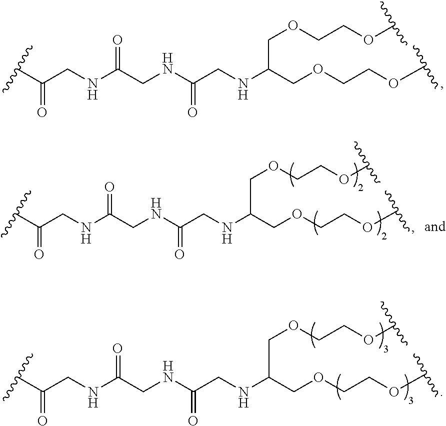

In certain embodiments Linker^{C} is selected from:

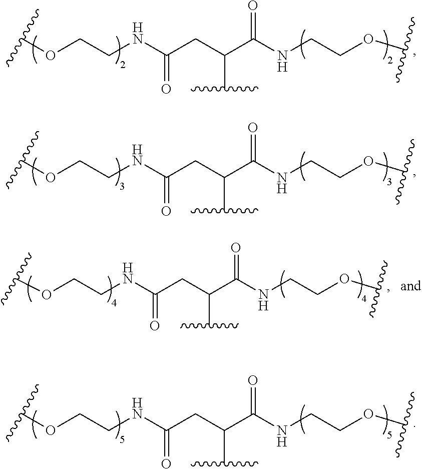

In certain embodiments Linker^{C} is selected from:

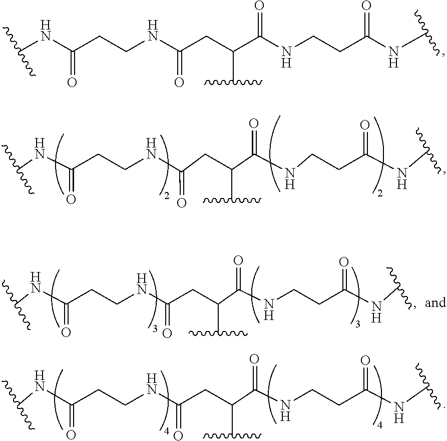

In certain embodiments Linker^{C} is selected from:

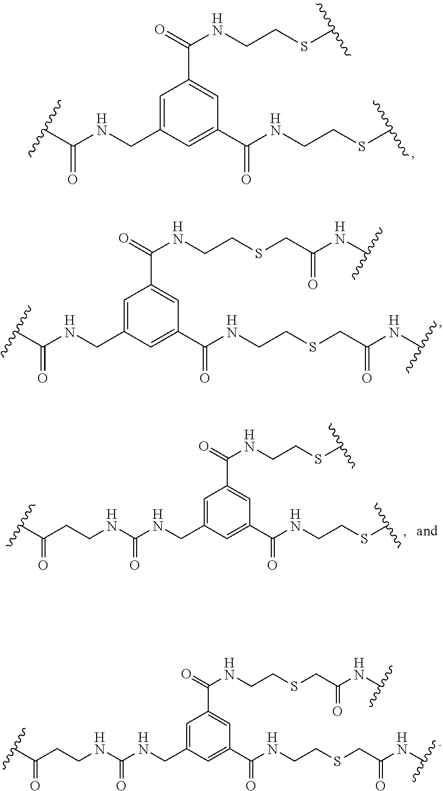 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments Linker^{C}-(Linker^{A})₂ is selected from:

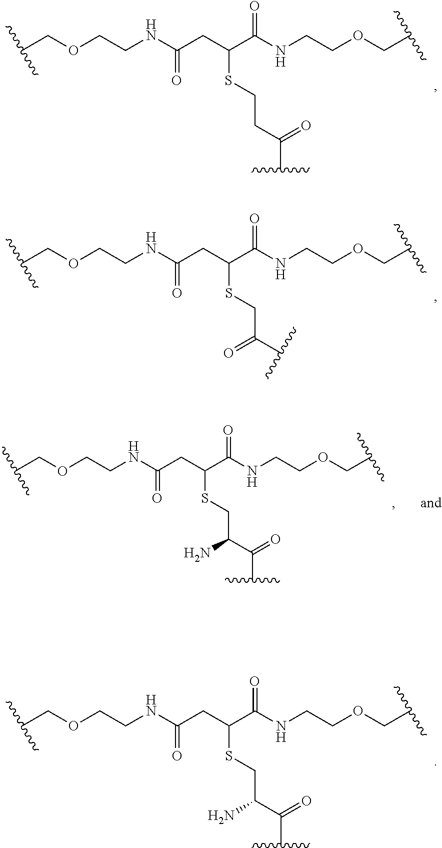 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments Linker^{C}-(Linker^{A})₂ is selected from:

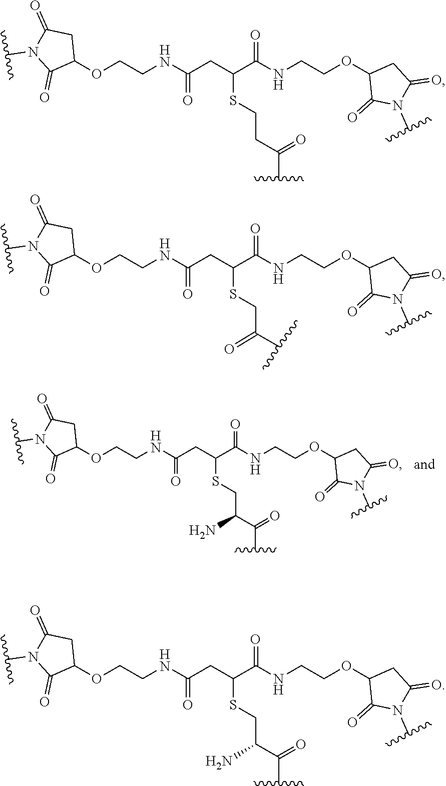 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments Linker^{C}-(Linker^{A})₂ is selected from:

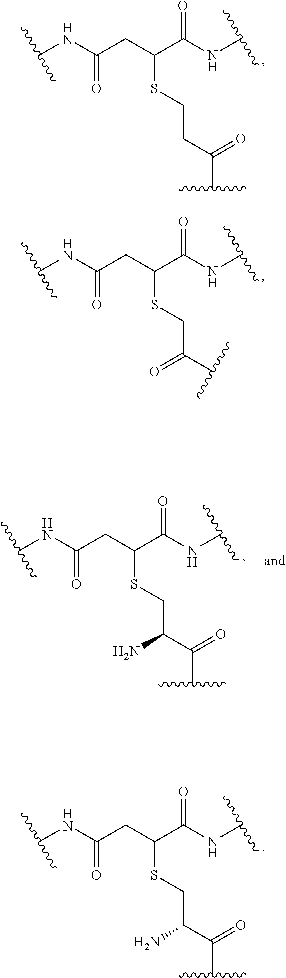

In certain embodiments Linker^{C}-(Linker^{A})₂ is selected from:

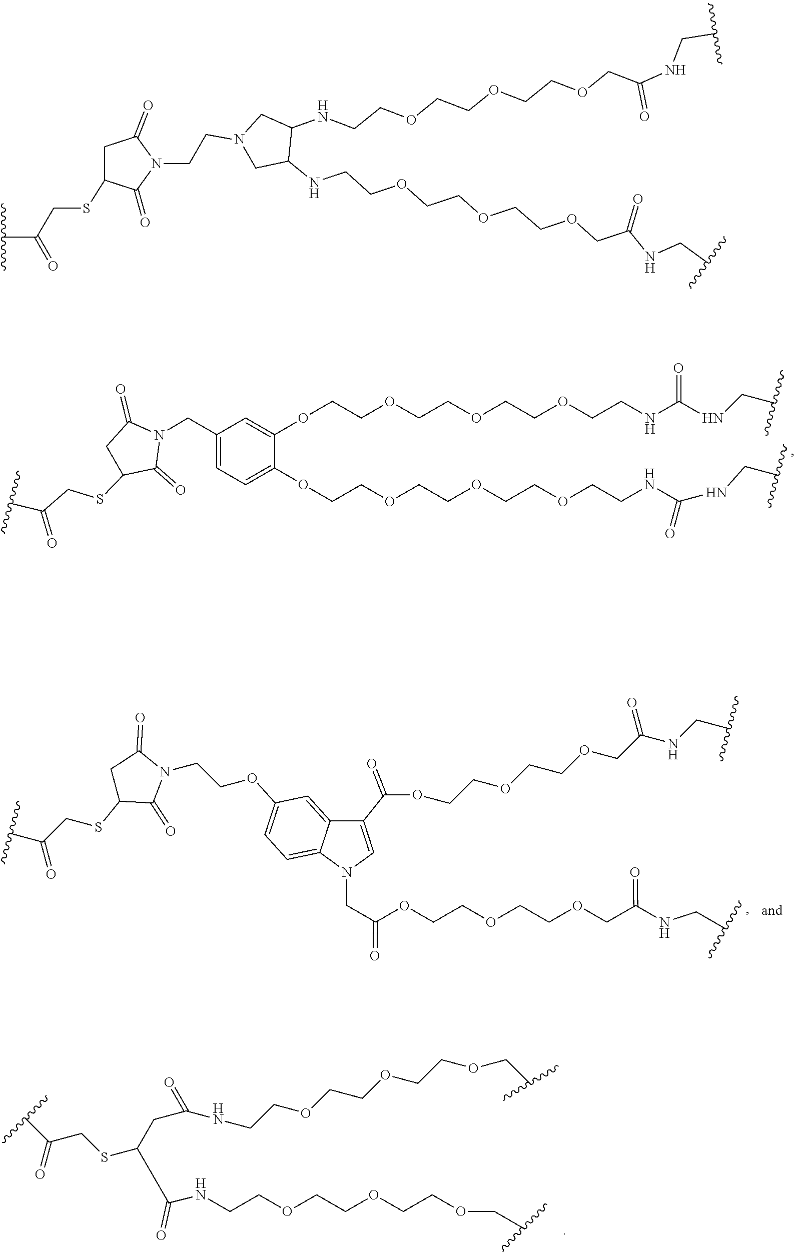 [SMILES: *CNC(=O)COCCOCCOCCNC1CN(CCN2C(=O)CC(SCC(*)=O)C2=O)CC1NCCOCCOCCOCC(=O)NC* ; *CNC(=O)NCCOCCOCCOCCOc1ccc(CN2C(=O)CC(SCC(*)=O)C2=O)cc1OCCOCCOCCOCCNC(=O)NC* ; *CNC(=O)COCCOCCOC(=O)Cn1cc(C(=O)OCCOCCOCC(=O)NC*)c2cc(OCCN3C(=O)CC(SCC(*)=O)C3=O)ccc21 ; *COCCOCCOCCNC(=O)CC(SCC(*)=O)C(=O)NCCOCCOCCOC*]

In certain embodiments, the Linker^{D} is selected from

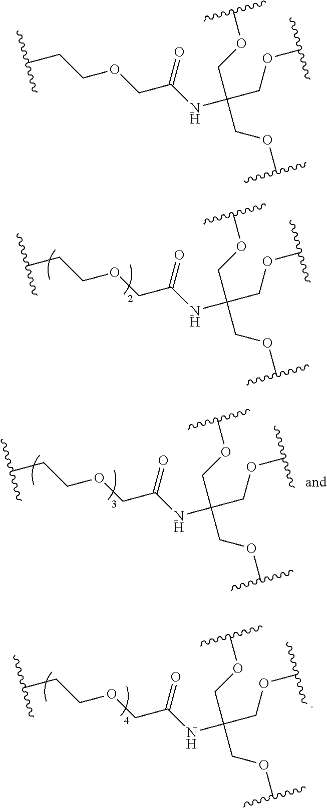

In certain embodiments, the Linker^{D} is selected from

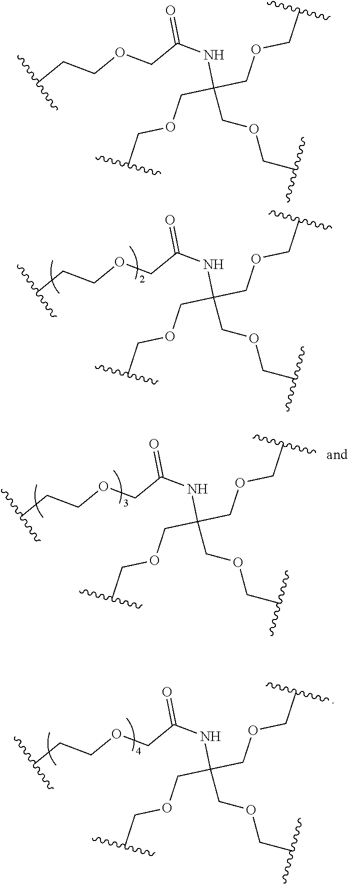

In certain embodiments, the Linker^{D} is selected from

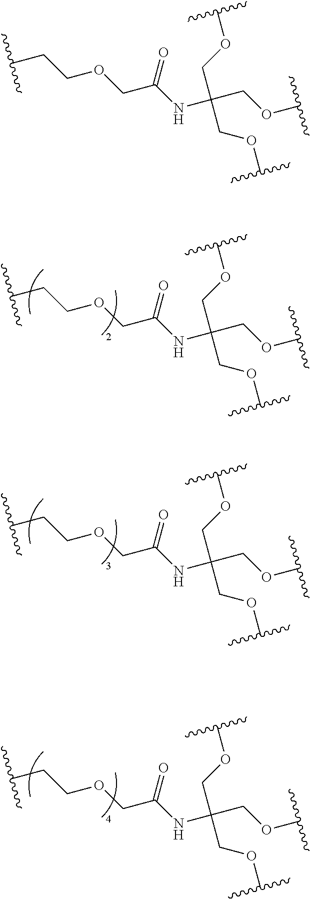

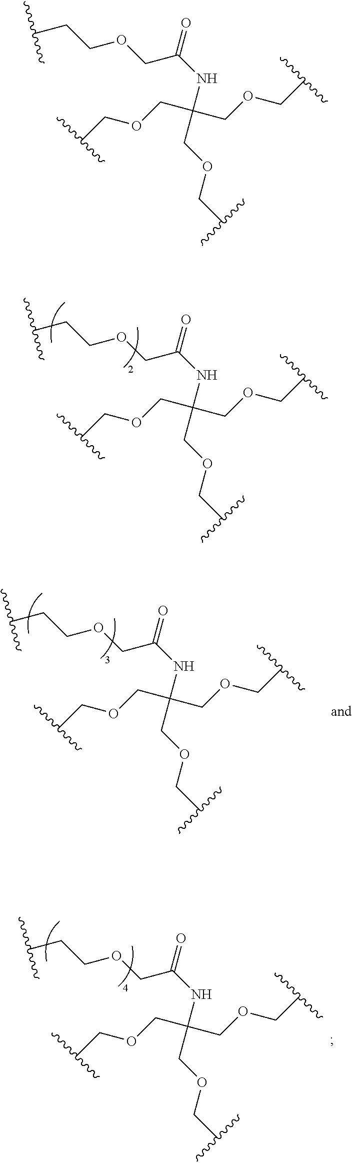

wherein each is optionally substituted with 1, 2, 3, or 4 substituents are selected from R²¹.

In certain embodiments, Linker^{B}-(Linker^{A}) is selected from

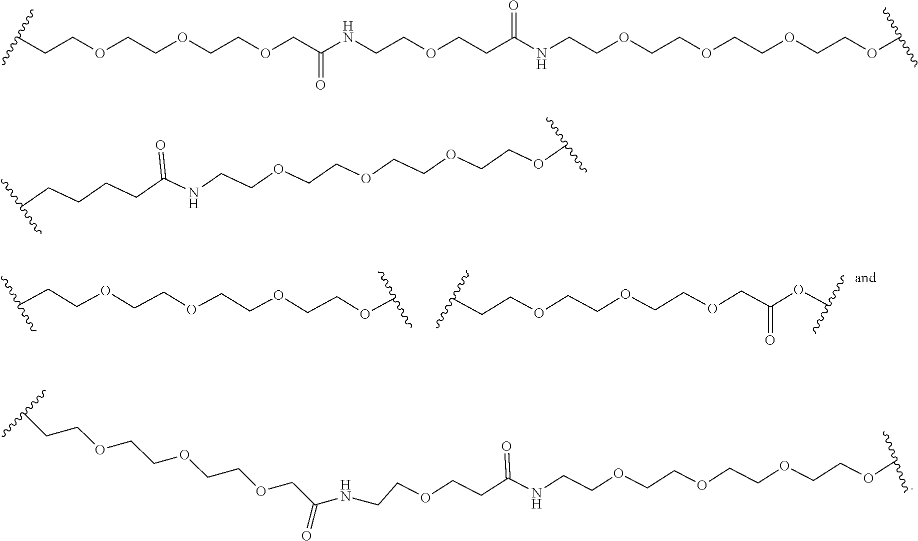 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, Linker^{C}-(Linker^{A}) is selected from

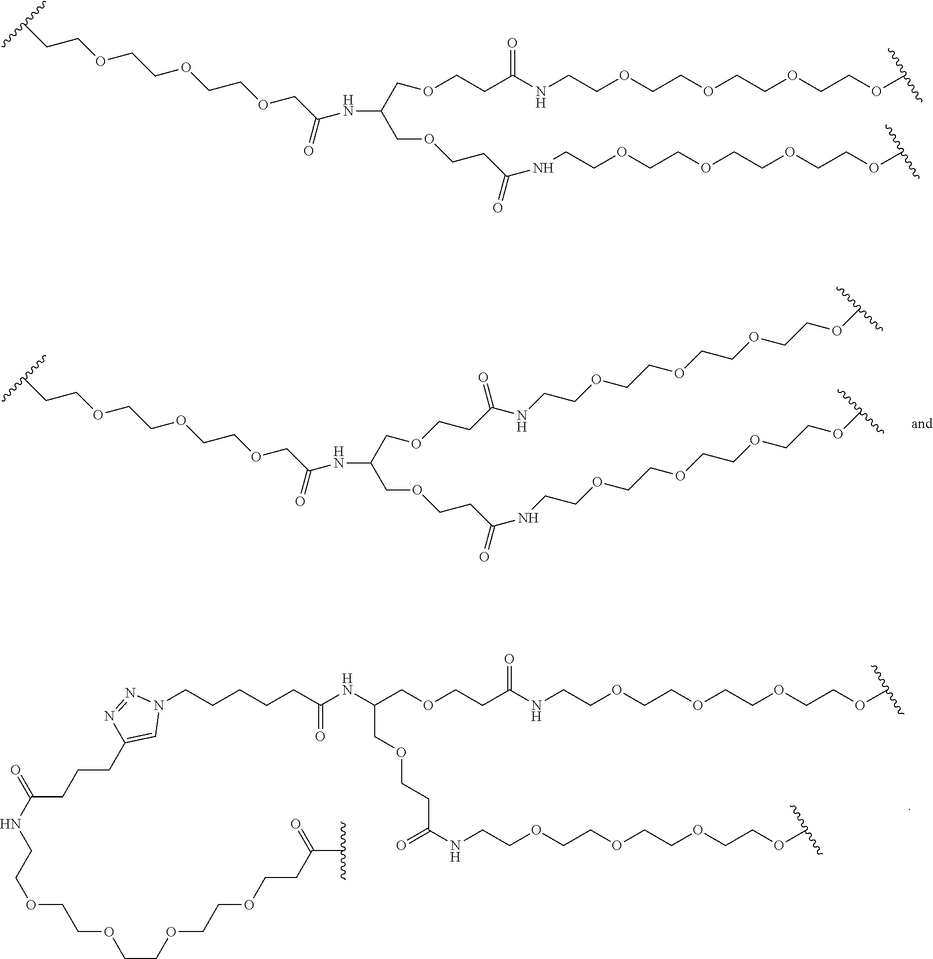 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, Linker^{D}-(Linker^{A}) is selected from

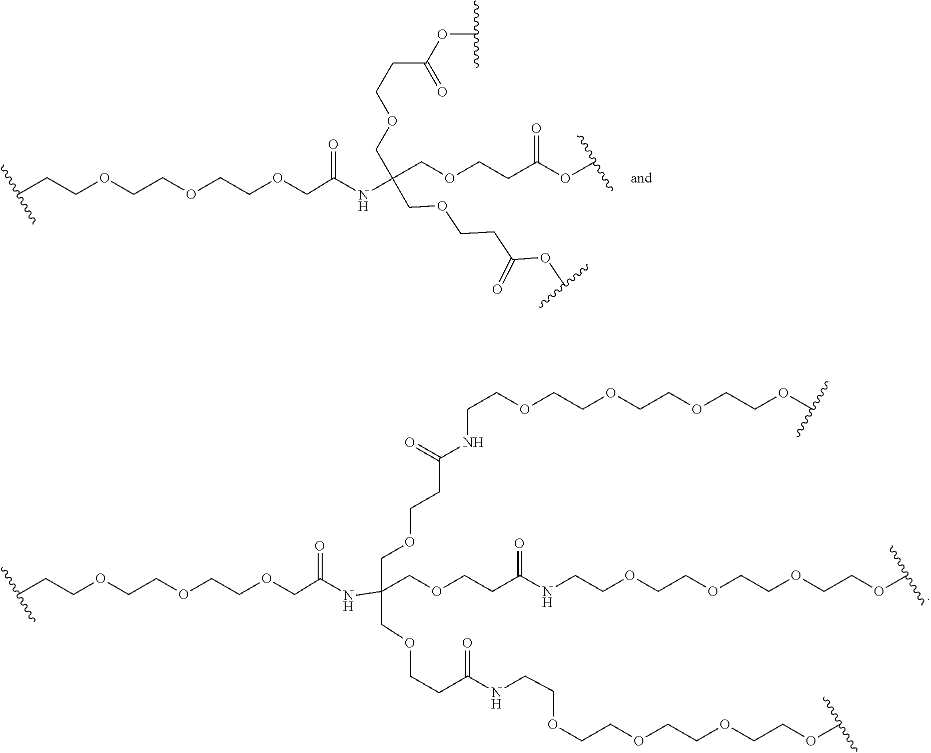 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In certain embodiments Linker^{B} is selected from:

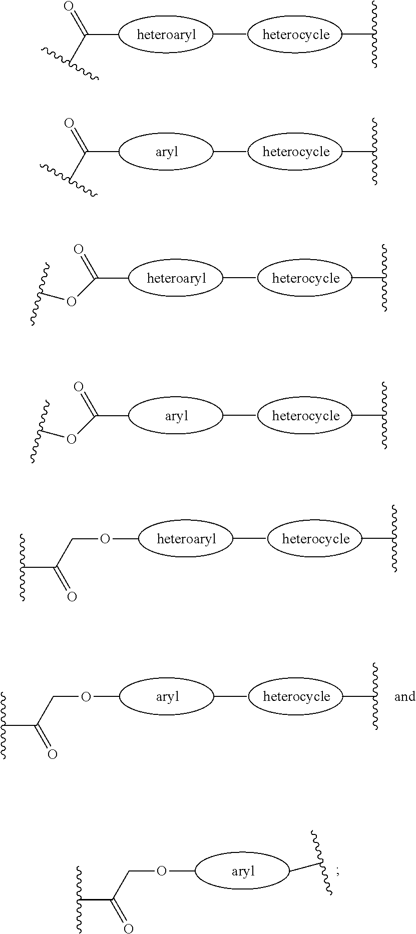

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

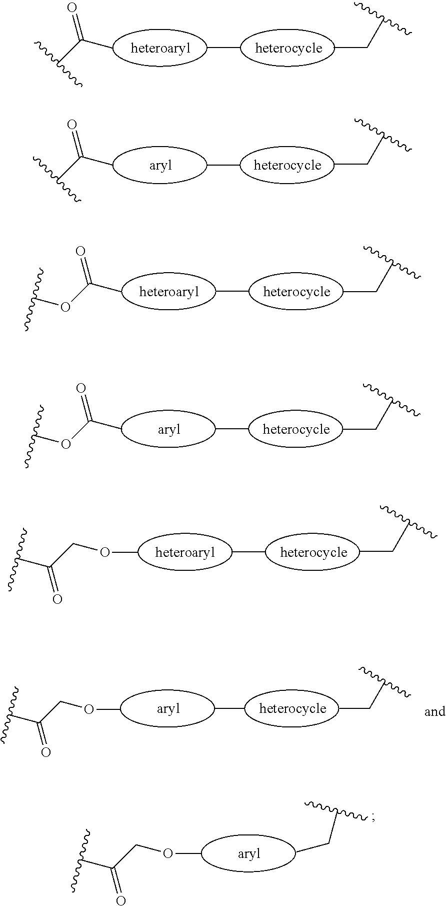

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

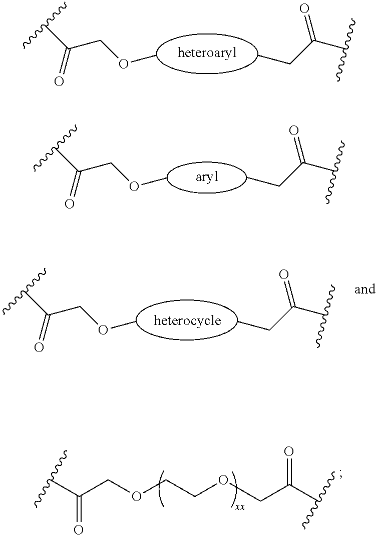

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B}, Linker^{C}, or Linker^{D} is selected from:

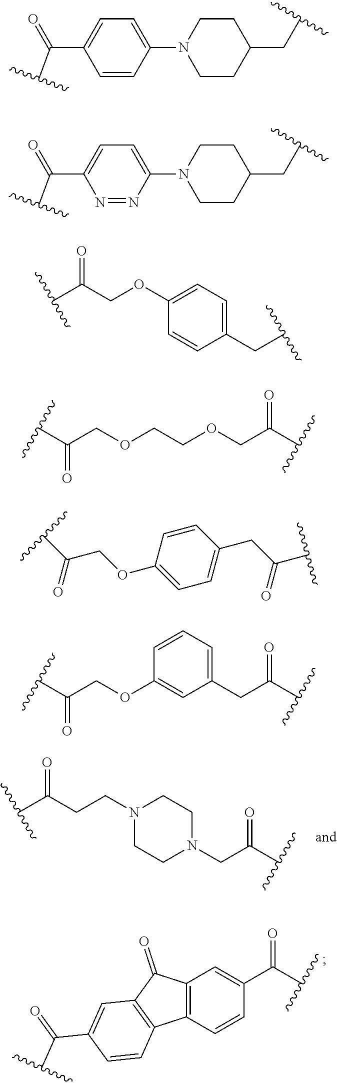

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

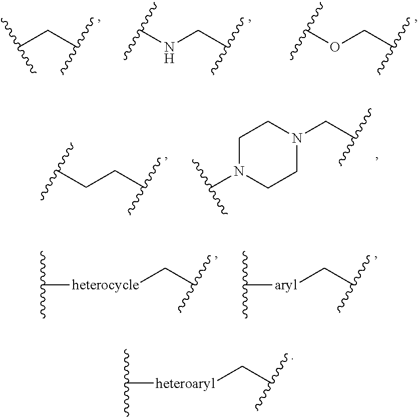

In certain embodiments Linker^{A} is selected from:

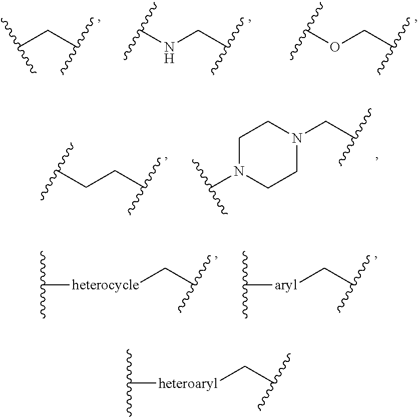

each of which is substituted with 1 or 2 optional substituents.

In certain embodiments Linker^{A} is bond.

In certain embodiments the left side of Linker^{A} is attached to the ASGPR Binding Ligand and the right side is attached to Linker^{B}, Linker^{C}, or Linker^{D}.

In certain embodiments the right side of Linker^{A} is attached to the ASGPR Binding Ligand and the right side is attached to Linker^{B}, Linker^{C}, or Linker^{D}.

In certain embodiments Linker^{B} is selected from:

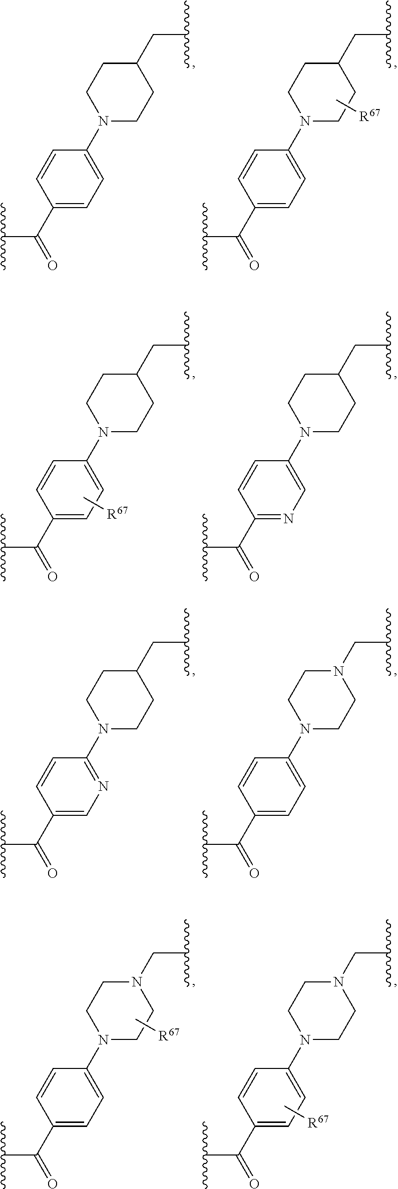

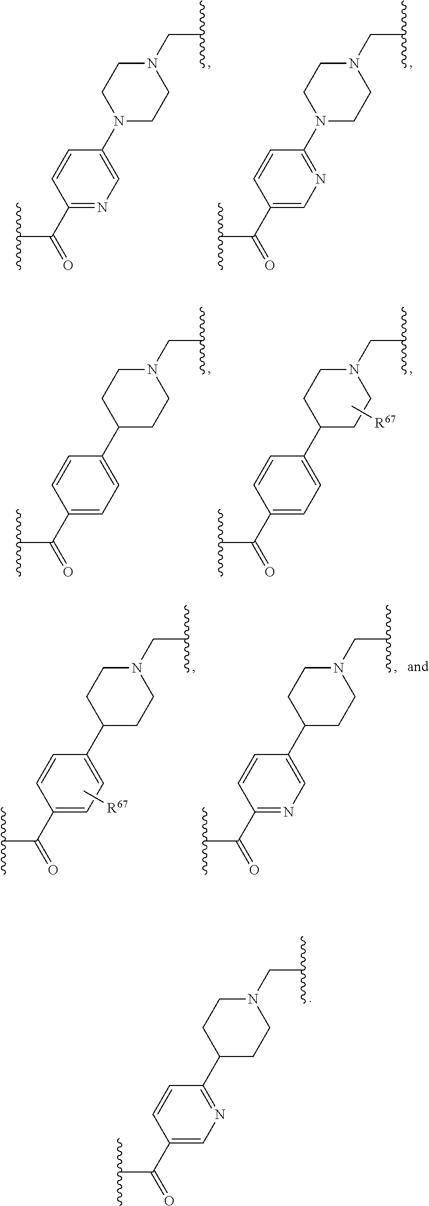

In certain embodiments Linker^{B} is selected from:

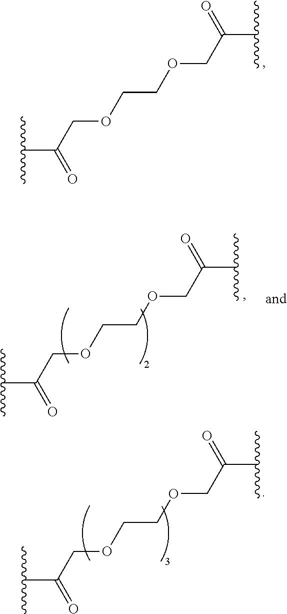

In certain embodiments the left side of Linker^{B} is attached to the Extracellular Targeting Ligand and the right side is attached to Linker^{A}.

In certain embodiments the right side of Linker^{B} is attached to the Extracellular Targeting Ligand and the left side is attached to Linker^{A}.

In certain embodiments Linker^{B} is bond.

In alternative embodiments a linker is provided as described above wherein a

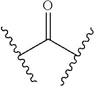 [SMILES: *C(*)=O]

is replaced with a

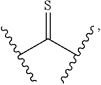 [SMILES: *C(*)=S]

for example where Linker^{B} is drawn as

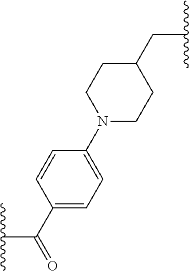 [SMILES: *CC1CCN(c2ccc(C(*)=O)cc2)CC1]

it is

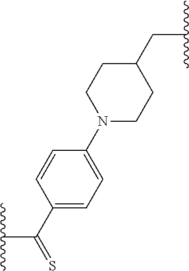 [SMILES: *CC1CCN(c2ccc(C(*)=S)cc2)CC1]

in this embodiment.

In alternative embodiments a linker is provided as described above wherein a

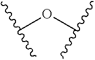 [SMILES: *O*]

is replaced with a

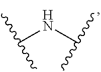 [SMILES: *N*]

for example where Linker^{B} is drawn as

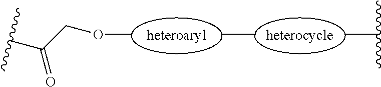

it is

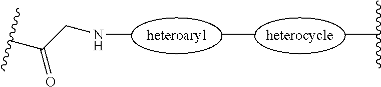

in this embodiment.

In alternative embodiments a linker is provided as described above wherein a

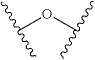 [SMILES: *O*]

is replaced with a

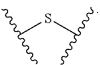 [SMILES: *S*]

### Additional Embodiments of Linker

In certain embodiments, the linker includes at least a linear group comprising groups selected from alkyl, amide, disulfide, polyethylene glycol, ether, thioether (—S—) and hydroxylamino (—O—N(H)—) groups. In certain embodiments, the linear group comprises groups selected from alkyl, amide and ether groups. In certain embodiments, the linear group comprises groups selected from alkyl and ether groups. In certain embodiments, the linear group comprises at least one phosphorus linking group. In certain embodiments, the linear group comprises at least one phosphodiester group. In certain embodiments, the linear group includes at least one neutral linking group.

In certain embodiments, Linker^{B} is selected from:

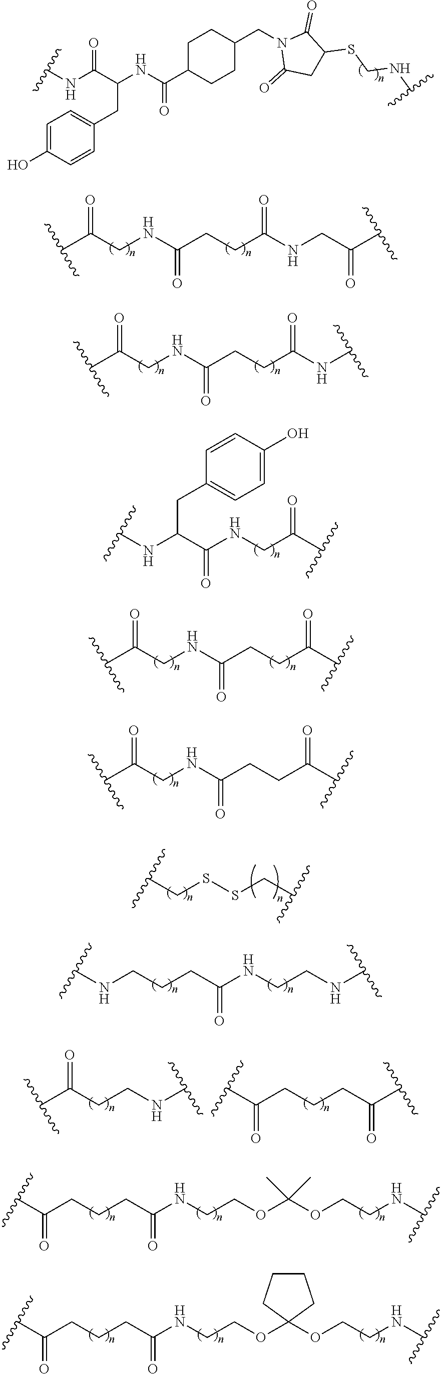

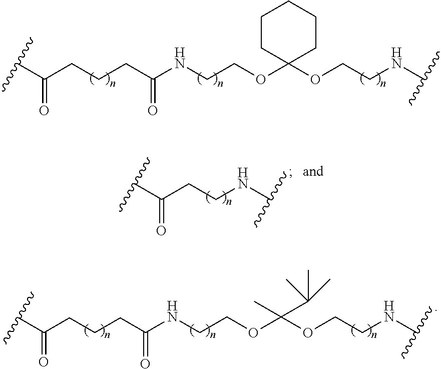

In certain embodiments, Linker^{B} is selected from:

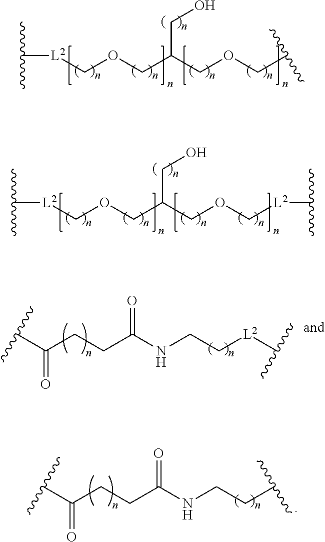

wherein each L² is independently a phosphorus linking group or a neutral linking group.

In certain embodiments, Linker^{B} is selected from:

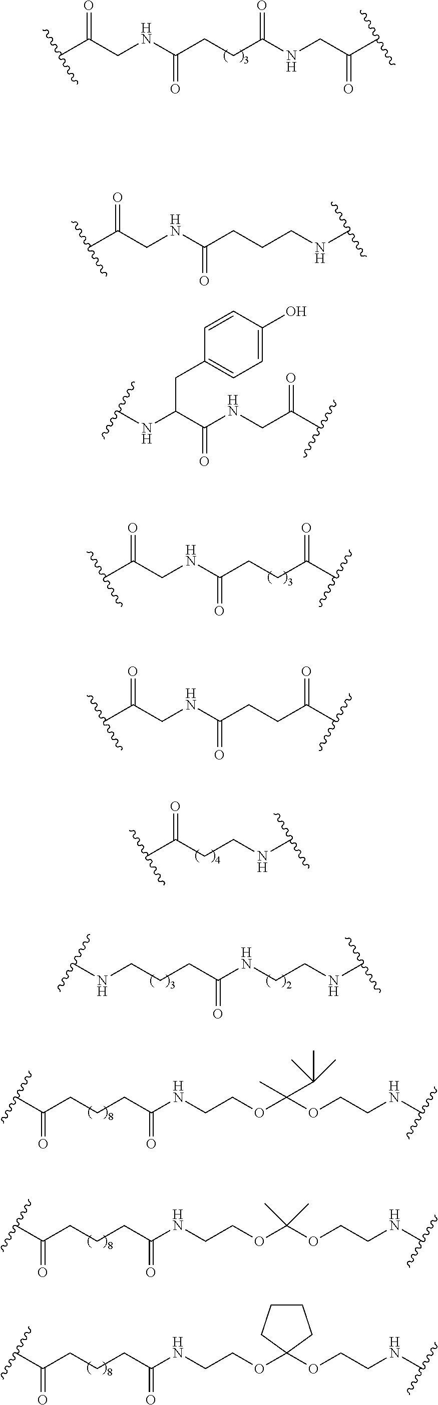

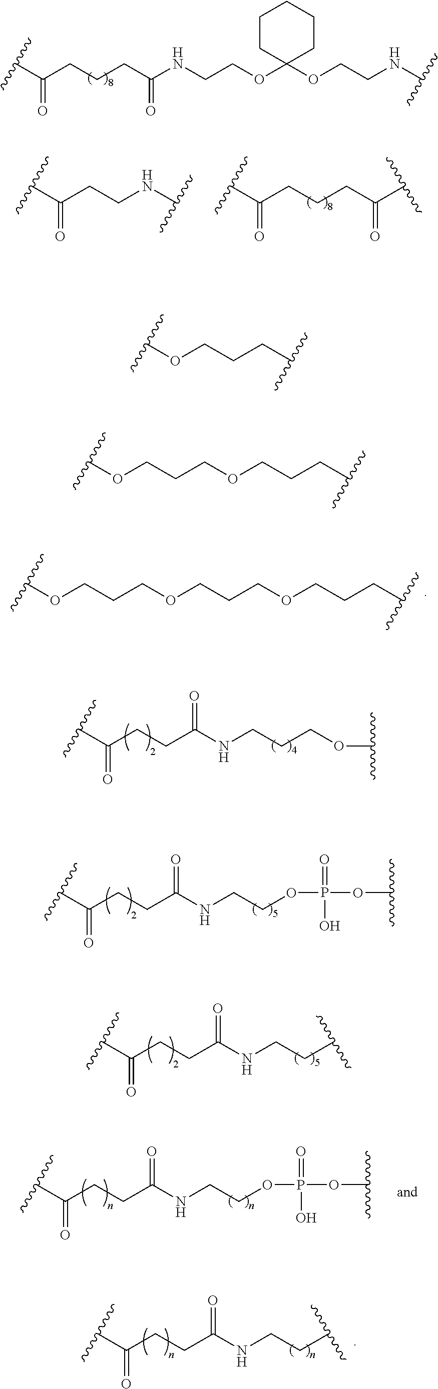

In certain embodiments, Linker^{B} is selected from:

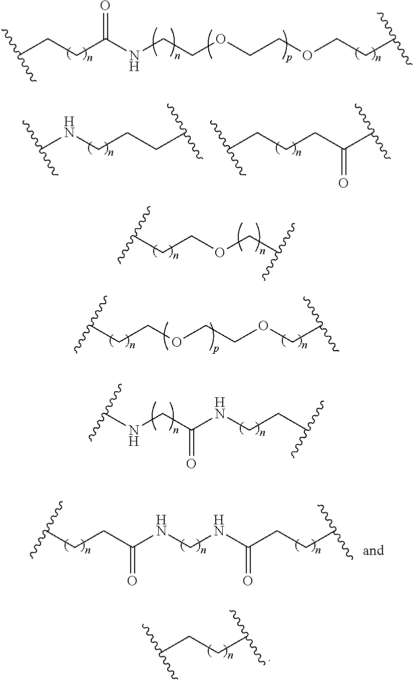

In certain embodiments, Linker^{C} is selected from:

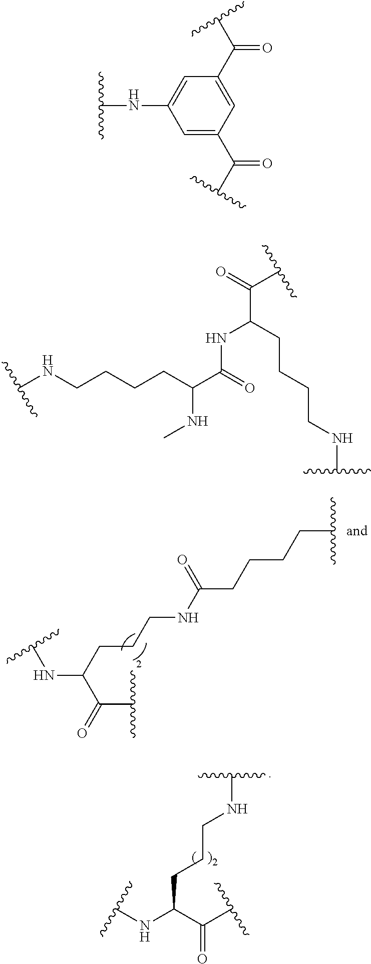

In certain embodiments, Linker^{C} is selected from:

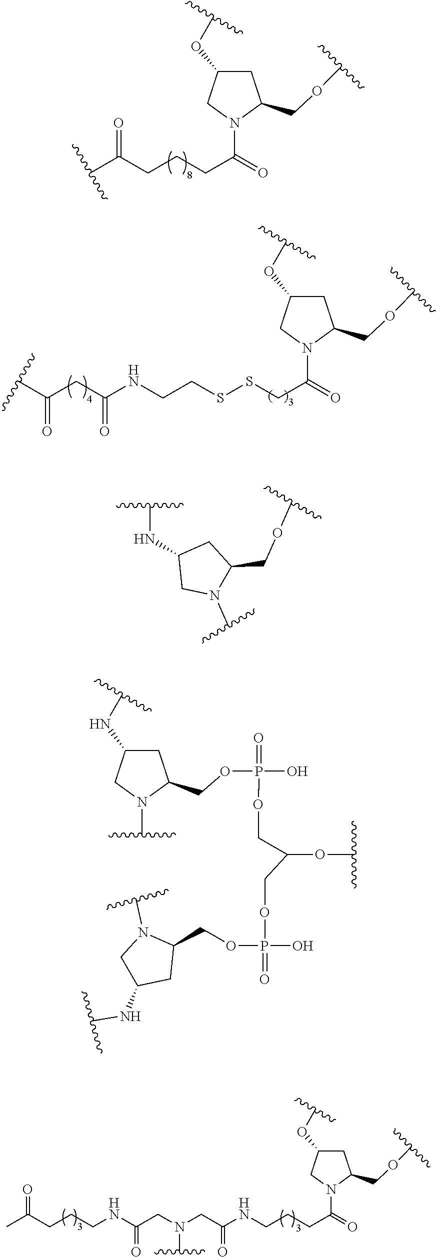

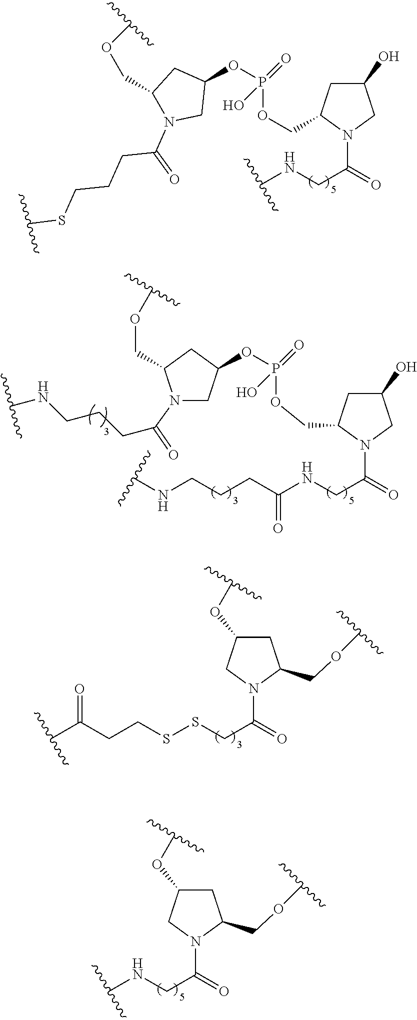

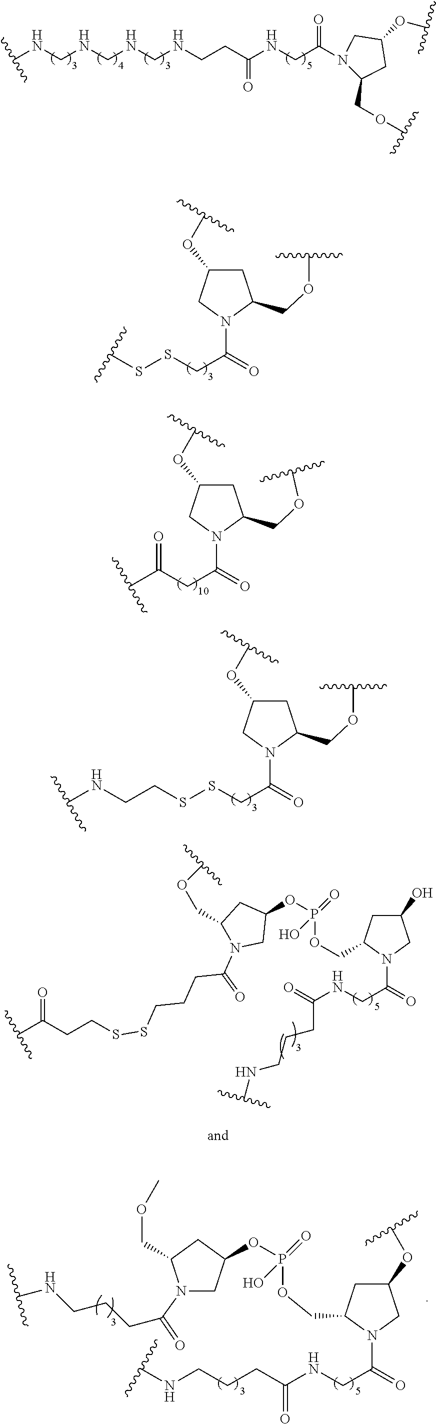

In certain embodiments, Linker^{C} is selected from:

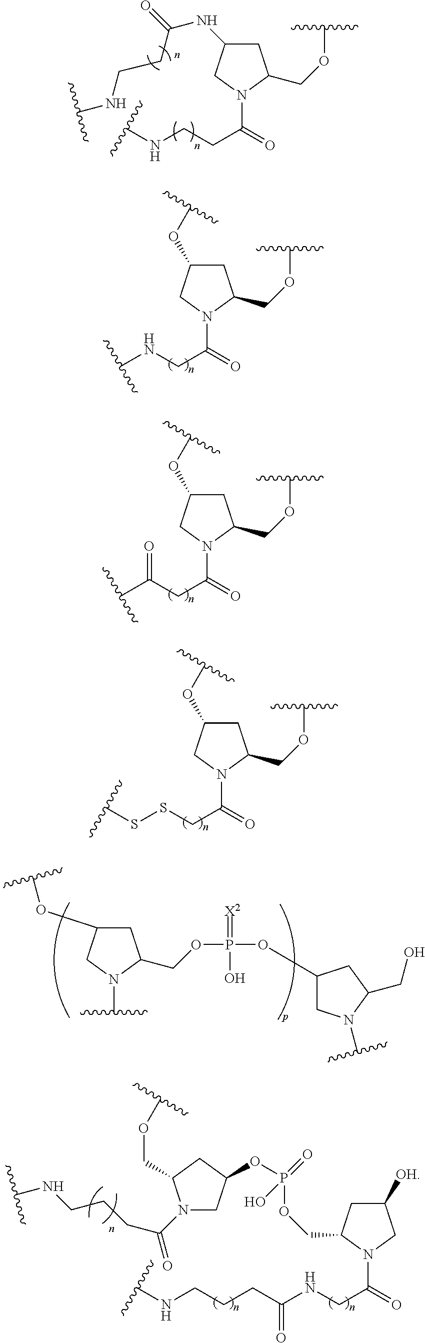

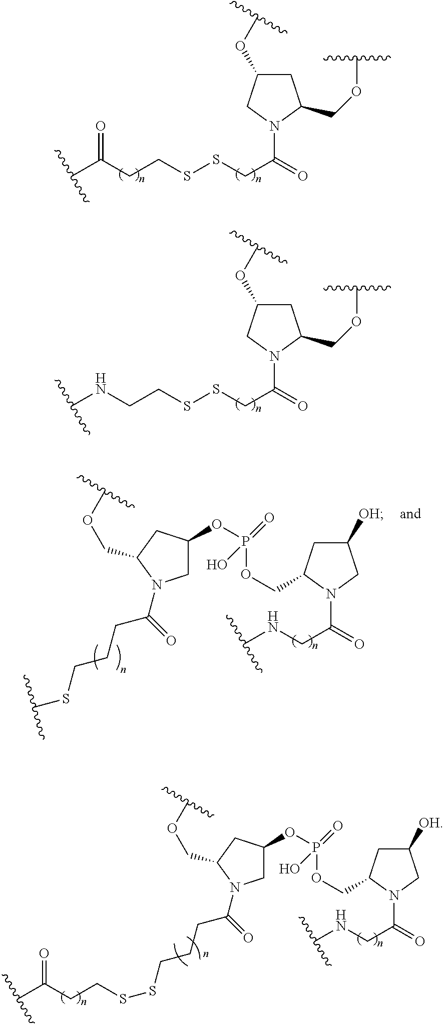

wherein each n is independently selected from 1 to 20, X² is O or S, and p is from 1 to 6.

In certain embodiments, Linker^{C} is selected from:

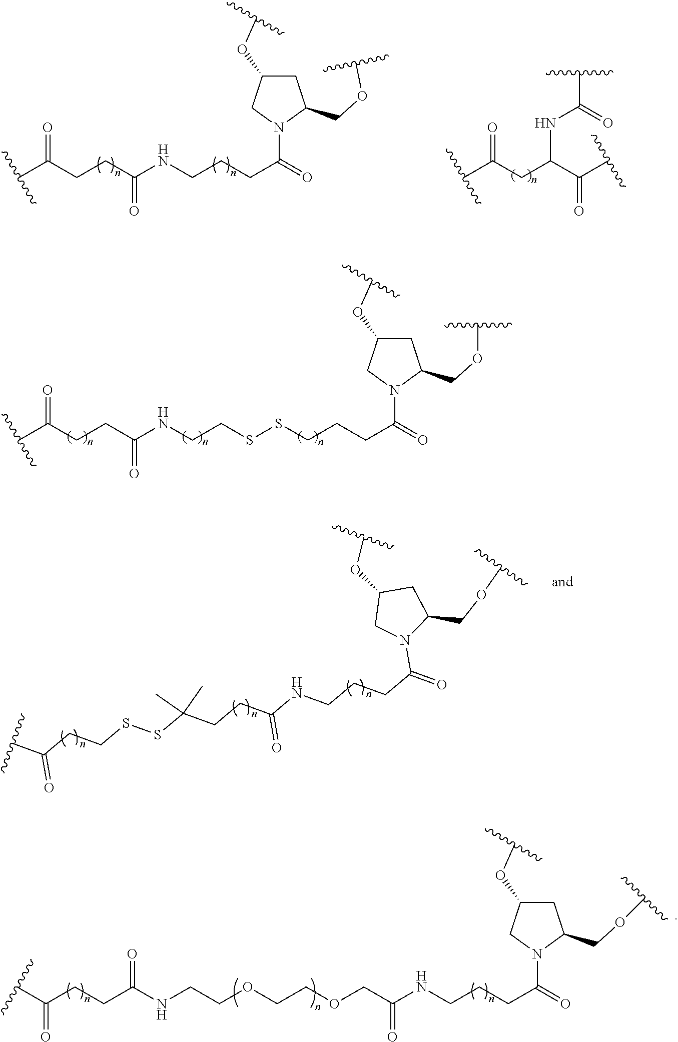

In certain embodiments, Linker^{C} is selected from:

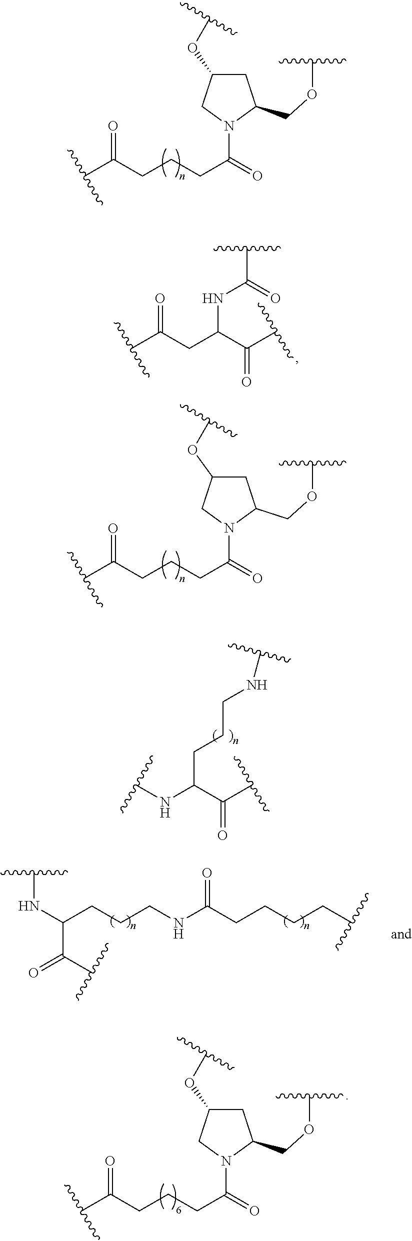

In certain embodiments, Linker^{D} is selected from:

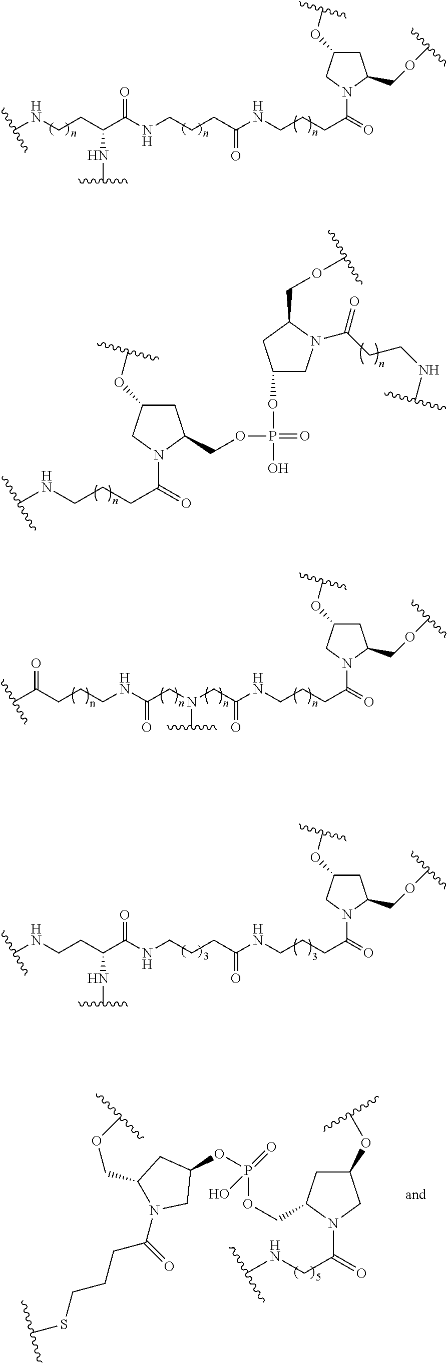

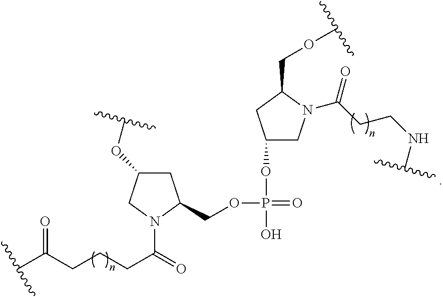

In certain embodiments, Linker^{D} is selected from:

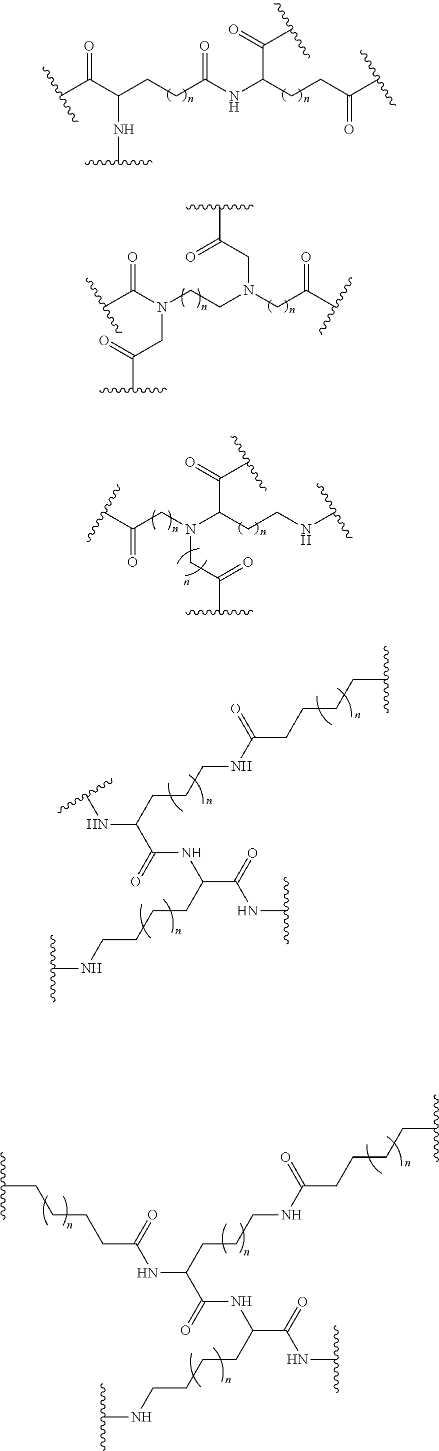

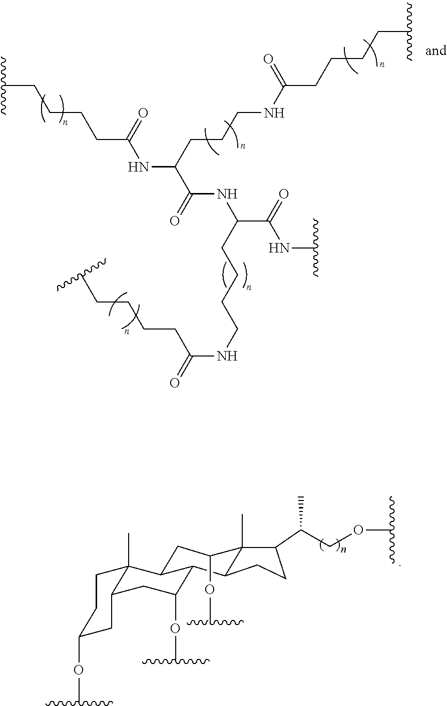

In certain embodiments, Linker^{D} is selected from:

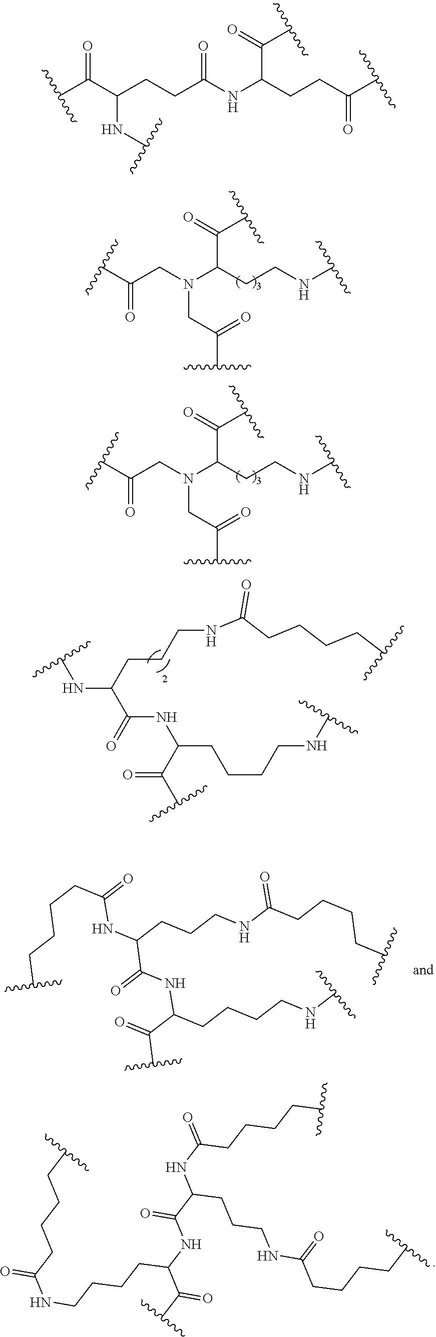

In certain embodiments n is independently 0, 1, 2, 3, 4, 5, 6, or 7.

In certain embodiments n is 1.

In certain embodiments n is 2.

In certain embodiments n is 3.

In certain embodiments n is 4.

In certain embodiments n is 5.

In certain embodiments n is 6.

In certain embodiments n is 7.

### Embodiments of ASGPR Binding Ligand

In certain embodiments ASGPR Binding Ligand is of Formula:

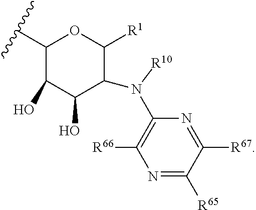

In certain embodiments ASGPR Binding Ligand is of Formula:

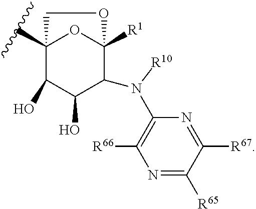

In certain embodiments ASGPR Binding Ligand is of Formula:

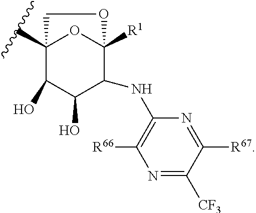

In certain embodiments ASGPR Binding Ligand is of Formula:

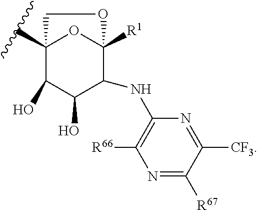

In certain embodiments ASGPR Binding Ligand is of Formula:

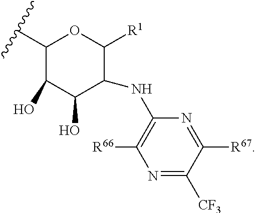

In certain embodiments ASGPR Binding Ligand is of Formula:

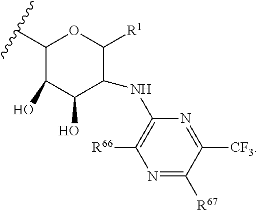

In certain embodiments ASGPR Binding Ligand is of Formula:

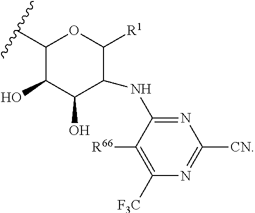

In certain embodiments ASGPR Binding Ligand is of Formula:

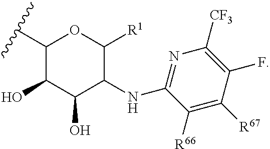

In certain embodiments ASGPR Binding Ligand is of Formula:

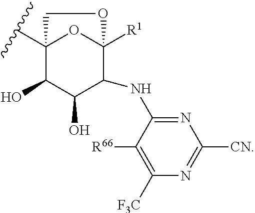

In certain embodiments ASGPR Binding Ligand is of Formula:

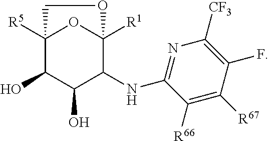

In certain embodiments ASGPR Binding Ligand is of Formula:

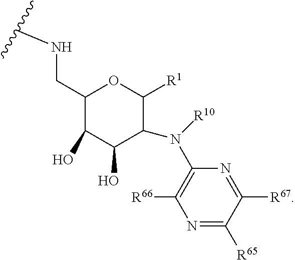

In certain embodiments ASGPR Binding Ligand is of Formula:

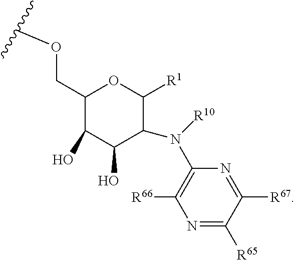

In certain embodiments ASGPR Binding Ligand is of Formula:

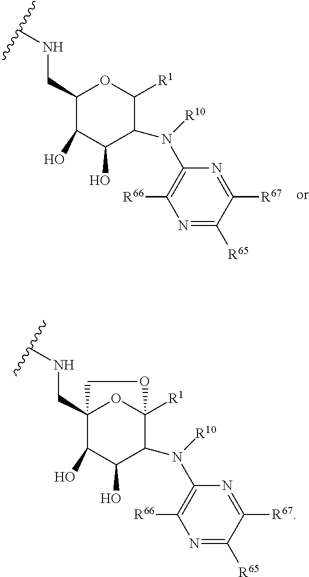

In certain embodiments ASGPR Binding Ligand is of Formula:

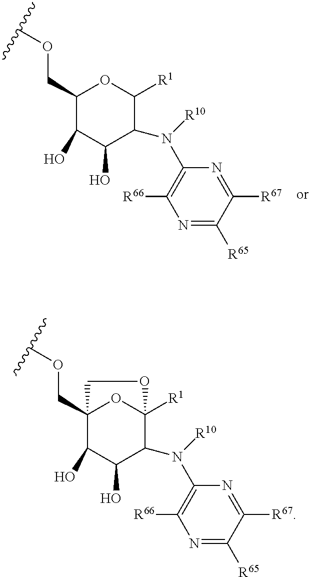

In certain embodiments ASGPR Binding Ligand is of Formula:

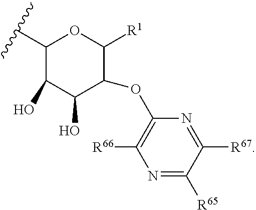

In certain embodiments ASGPR Binding Ligand is of Formula:

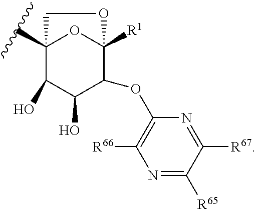

In certain embodiments ASGPR Binding Ligand is of Formula:

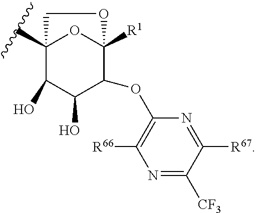

In certain embodiments ASGPR Binding Ligand is of Formula:

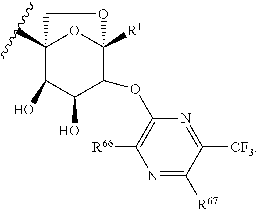

In certain embodiments ASGPR Binding Ligand is of Formula:

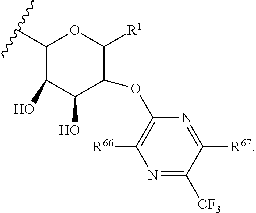

In certain embodiments ASGPR Binding Ligand is of Formula:

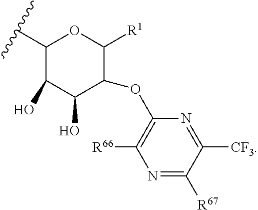

In certain embodiments ASGPR Binding Ligand is of Formula:

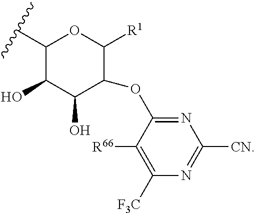

In certain embodiments ASGPR Binding Ligand is of Formula:

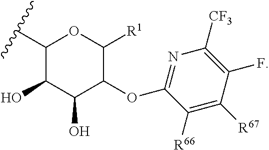

In certain embodiments ASGPR Binding Ligand is of Formula:

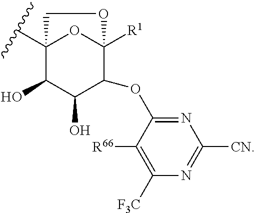

In certain embodiments ASGPR Binding Ligand is of Formula:

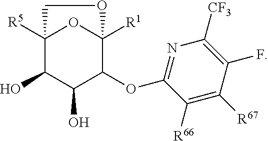

In certain embodiments ASGPR Binding Ligand is of Formula:

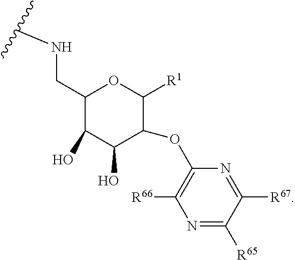

In certain embodiments ASGPR Binding Ligand is of Formula:

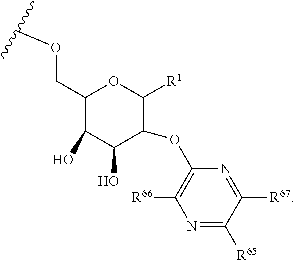

In certain aspects the Therapeutic Oligonucleotide delivering compound is of Formula

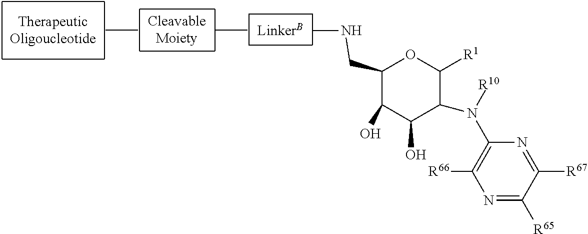

or a pharmaceutically acceptable salt thereof.

In certain aspects the Therapeutic Oligonucleotide delivering compound is of Formula

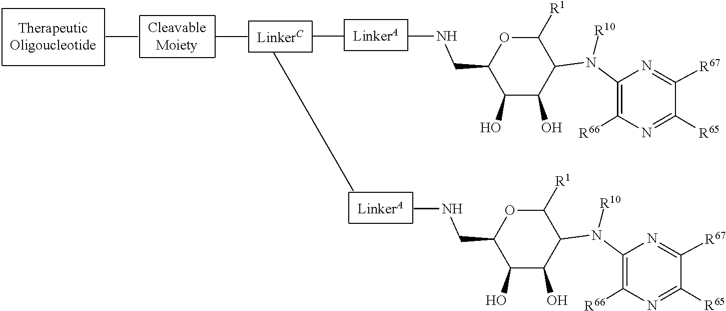

or a pharmaceutically acceptable salt thereof.

In certain aspects the Therapeutic Oligonucleotide delivering compound is of Formula

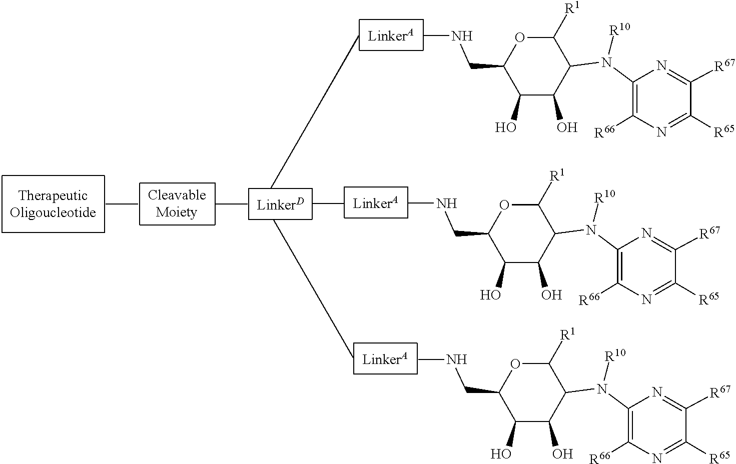

or a pharmaceutically acceptable salt thereof.

In certain aspects the Therapeutic Oligonucleotide delivering compound is of Formula

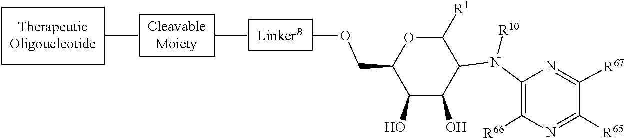

or a pharmaceutically acceptable salt thereof.

In certain aspects the Therapeutic Oligonucleotide delivering compound is of Formula

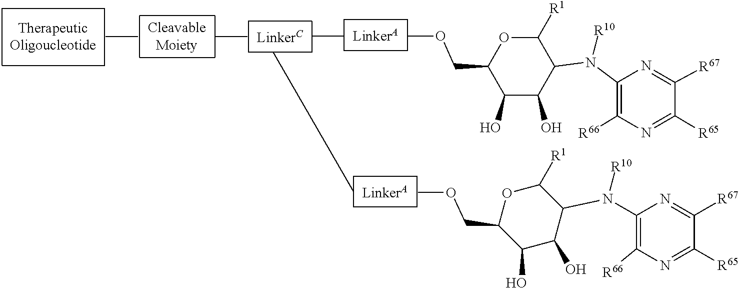

or a pharmaceutically acceptable salt thereof.

In certain aspects the Therapeutic Oligonucleotide delivering compound is of Formula

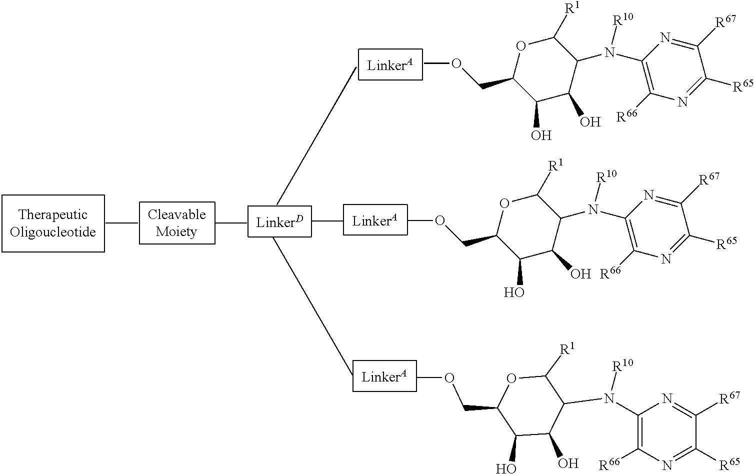

or a pharmaceutically acceptable salt thereof.

In certain embodiments ASGPR Binding Ligand is of Formula:

wherein R* is selected from:

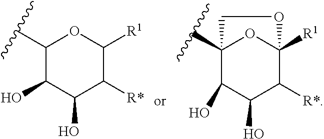

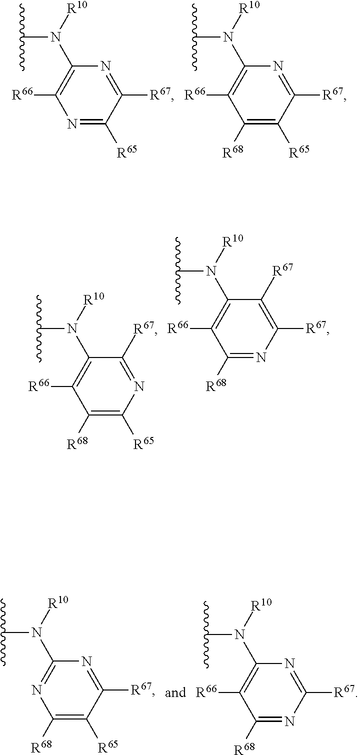

In certain embodiments the ASGPR Binding Ligand is of Formula:

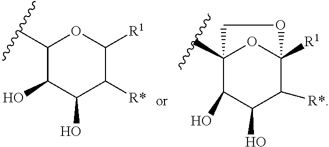

In certain embodiments R* is selected from:

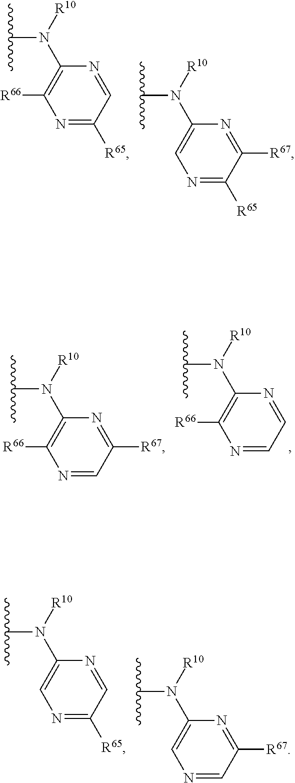

Non limiting examples of R* include:

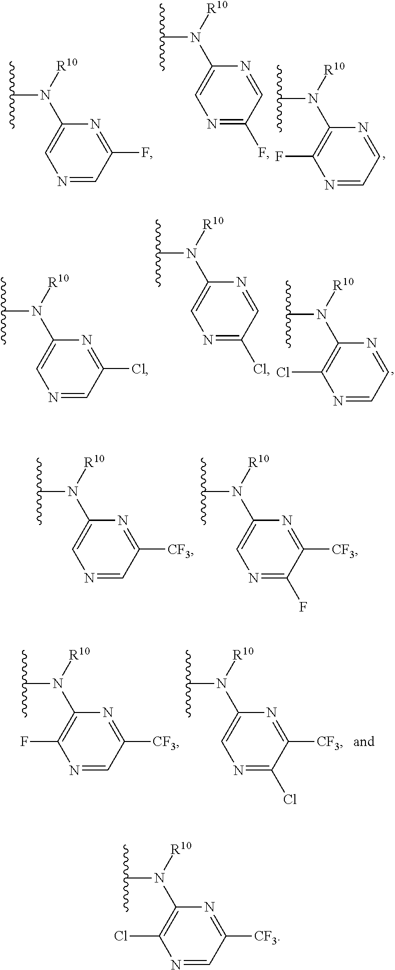

In certain embodiments, ASGPR Binding Ligand is selected from

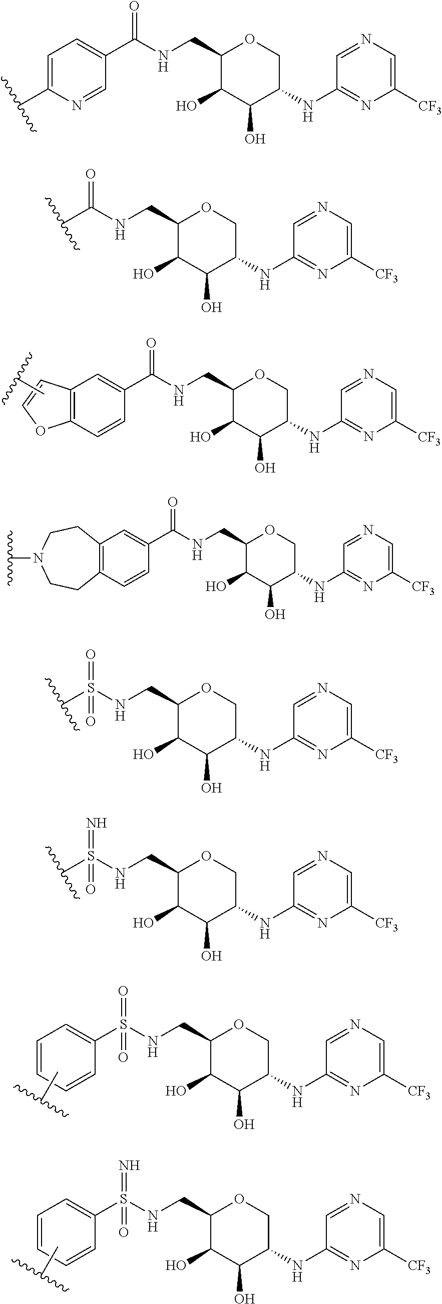

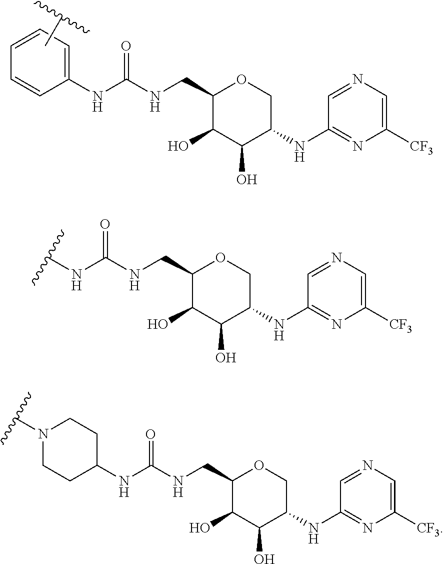

In certain embodiments, ASGPR Binding Ligand is selected from

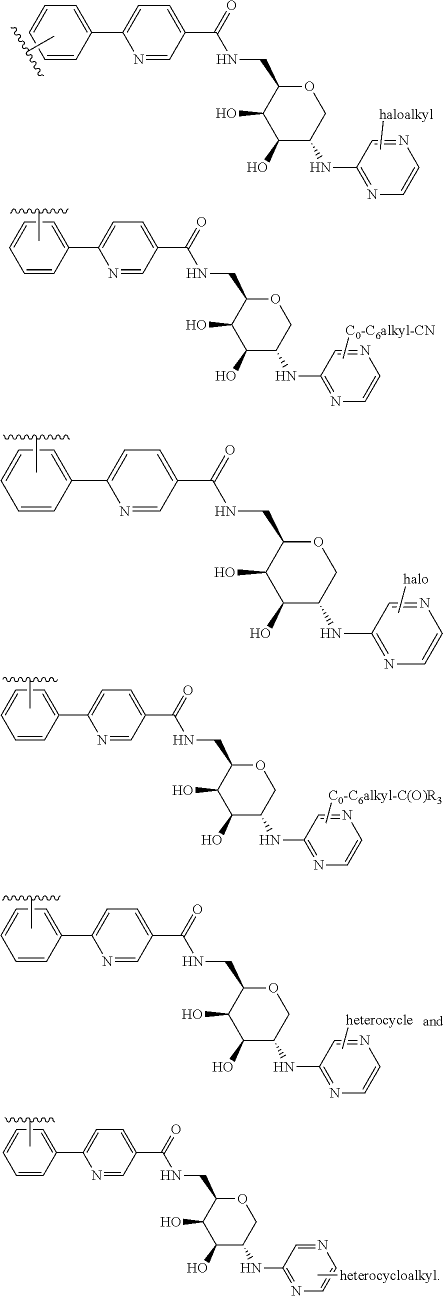

In certain embodiments, ASGPR Binding Ligand is selected from:

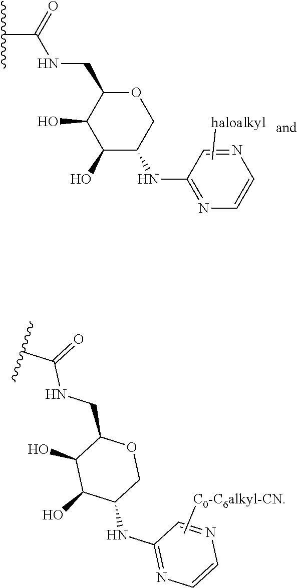

In certain embodiments the Therapeutic Oligonucleotide delivering compound is selected from:

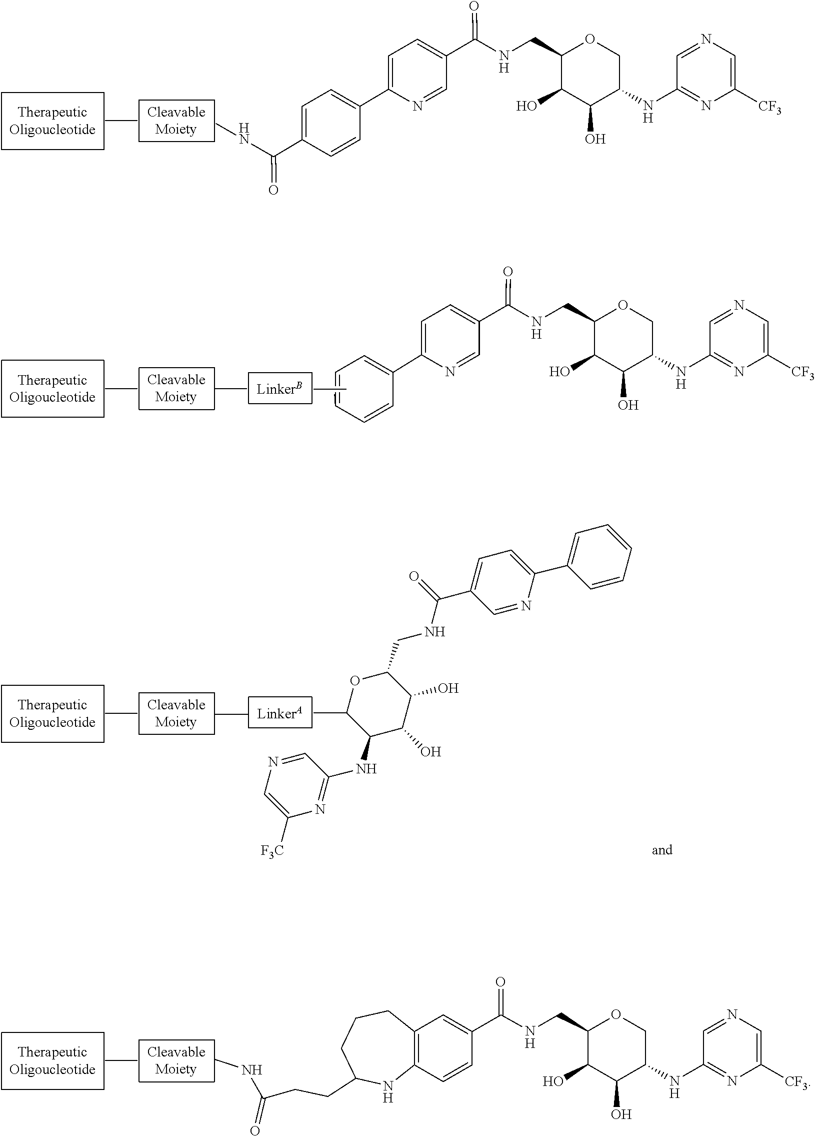

### Additional Embodiments of the ASGPR Binding Ligand

In certain embodiments, R¹ is selected from hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R′)—C(O)R³, C₀-C₆alkyl-N(R′)—S(O)R³, C₀-C₀-C₆alkyl-N(R′)—S(O)₂R³, C₀-C₆alkyl-O—C(O)R³, C₀-C₆alkyl-O—S(O)R³, —N═S(O)(R³)₂, C₀-C₆alkylN₃, or C₀-C₆alkyl-O—S(O)₂R³.

In certain embodiments, R¹ is selected from hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R′)—C(O)R³, C₀-C₆alkyl-N(R′)—S(O)R³, C₀-C₆alkyl-O—C(O)R³, and C₀-C₆alkyl-O—S(O)R³.

In certain embodiments, R¹ is selected from hydrogen, alkyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R⁸)—C(O)R³, C₀-C₆alkyl-N(R′)—S(O)R³, C₀-C₆alkyl-O—C(O)R³, and C₀-C₆alkyl-O—S(O)R³.

In certain embodiments, R¹ is selected from C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R⁸)—C(O)R³, C₀-C₆alkyl-N(R′)—S(O)R³, C₀-C₀-C₆alkyl-N(R⁸)—S(O)₂R³, C₀-C₆alkylO—C(O)R³, C₀-C₆alkyl-O—S(O)R³, —N═S(O)(R³)₂, C₀-C₆alkylN₃, or C₀-C₆alkyl-O—S(O)₂R³.

In certain embodiments, R⁵ is selected from hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R′)—C(O)R³, C₀-C₆alkyl-N(R′)—S(O)R³, C₀-C₀-C₆alkyl-N(R⁸)—S(O)₂R³, C₀-C₆alkyl-O—C(O)R³, C₀-C₆alkyl-O—S(O)R³, —N═S(O)(R³)₂, C₀-C₆alkylN₃, or C₀-C₆alkyl-O—S(O)₂R³.

In certain embodiments, R⁵ is selected from hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R′)—C(O)R³, C₀-C₆alkyl-N(R′)—S(O)R³, C₀-C₆alkyl-O—C(O)R³, and C₀-C₆alkyl-O—S(O)R³.

In certain embodiments, R⁵ is selected from hydrogen, alkyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R⁸)—C(O)R³, C₀-C₆alkyl-N(R′)—S(O)R³, C₀-C₆alkyl-O—C(O)R³, and C₀-C₆alkyl-O—S(O)R³.

In certain embodiments, R⁵ is selected from C₂-C₆alkyl-cyano, C₂-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-S(O)₂R³, C₀-C₆alkyl-N(R⁸)—C(O)R³, C₀-C₆alkyl-N(R⁸)—S(O)R³, C₀-C₀-C₆alkyl-N(R⁸)—S(O)₂R³, C₀-C₆alkylO—C(O)R³, C₀-C₆alkyl-O—S(O)R³, —N═S(O)(R³)₂, C₀-C₆alkylN₃, or C₀-C₆alkyl-O—S(O)₂R³.

In certain embodiments, R³ at each occurrence is independently selected from the group consisting of alkyl, haloalkyl, arylalkyl, heteroarylalkyl, alkenyl, alkynyl, aryl, heteroaryl, heterocycle, —OR⁸, and —NR⁸R⁹.

In certain embodiments, R³ at each occurrence is independently selected from the group consisting of alkyl, haloalkyl, arylalkyl, heteroarylalkyl, aryl, heteroaryl, heterocycle, —OR⁸, and —NR⁸R⁹.

In certain embodiments, R³ at each occurrence is independently selected from the group consisting of alkyl, haloalkyl, aryl, heteroaryl, heterocycle, —OR⁸, and —NR⁸R⁹.

In certain embodiments, R³ at each occurrence is independently selected from the group consisting of —OR⁸ and —NR⁸R⁹.

In certain embodiments, R³ at each occurrence is independently selected from the group consisting of alkyl, haloalkyl, arylalkyl, heteroarylalkyl, alkenyl, alkynyl, aryl, heteroaryl, and heterocycle.

In certain embodiments, R⁶ and R⁷ are independently selected at each occurrence from the group consisting of alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³.

In certain embodiments, R⁶ and R⁷ are independently selected at each occurrence from the group consisting of hydrogen, alkyl, arylalkyl, heteroarylalkyl, alkenyl, alkynyl, aryl, haloalkyl, heteroaryl, and heterocycle.

In certain embodiments, R⁶ and R⁷ are independently selected at each occurrence from the group consisting of C(O)R³, S(O)R³, C(S)R³, and S(O)₂R³.

In certain embodiments, R⁶ and R⁷ are independently selected at each occurrence from the group consisting of hydrogen, alkyl, aryl, heteroaryl, heterocycle, -alkyl-OR⁸, -alkyl-NR⁸R⁹, and C(O)R³.

In certain embodiments, R⁸ and R⁹ are independently selected at each occurrence from hydrogen, alkyl, arylalkyl, heteroarylalkyl, alkenyl, alkynyl, aryl, heteroaryl, and heterocycle.

In certain embodiments, R⁸ and R⁹ are independently selected at each occurrence from alkyl, arylalkyl, heteroarylalkyl, alkenyl, alkynyl, aryl, heteroaryl, and heterocycle.

In certain embodiments, R⁸ and R⁹ are independently selected at each occurrence from hydrogen, alkyl, aryl, heteroaryl, and heterocycle.

In certain embodiments, R¹⁰ is hydrogen, alkyl, arylalkyl, heteroarylalkyl, aryl, heteroaryl, heterocycle, C(O)R³, S(O)R³, C(S)R³, or S(O)₂R³.

In certain embodiments, R¹⁰ is alkyl, haloalkyl, arylalkyl, heteroarylalkyl, alkenyl, alkynyl, aryl, heteroaryl, heterocycle, C(O)R³, S(O)R³, C(S)R³, or S(O)₂R³.

In certain embodiments, R¹⁰ is hydrogen, alkyl, aryl, heteroaryl, heterocycle, C(O)R³, S(O)R³, C(S)R³, or S(O)₂R³.

In certain embodiments, R¹⁰ is hydrogen, alkyl, aryl, heteroaryl, or heterocycle.

In certain embodiments, R¹⁰ is hydrogen, alkyl, or aryl.

In certain embodiments, R¹⁰ is C(O)R³, S(O)R³, C(S)R³, or S(O)₂R³.

In certain embodiments, R⁶⁵, R⁶⁶, and R⁶⁷ are independently selected at each occurrence from the group consisting of hydrogen, C₀-C₆alkyl-cyano, haloalkyl, F, Cl, Br, I, heterocycle, C₀-C₆alkyl-C(O)R³, C₀-C₆alkyl-S(O)R³, C₀-C₆alkyl-S(O)₂R³, C₀-C₆alkyl-N(R′)—C(O)R³, C₀-C₆alkyl-N(R⁸)—S(O)R³, C₀-C₆alkyl-N(R′)—S(O)₂R³C₀-C₆alkyl-O—C(O)R³, C₀-C₆alkyl-O—S(O)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰.

In certain embodiments, R⁶⁵, R⁶⁶, and R⁶⁷ are independently selected at each occurrence from the group consisting of hydrogen, C₀-C₆alkyl-cyano, haloalkyl, F, Cl, Br, I, heterocycle, C₀-C₆alkyl-C(O)R³, C₀-C₆alkyl-N(R′)—C(O)R³, and C₀-C₆alkyl-O—C(O)R³, each of which is optionally substituted with 1, 2, or 3 substituents independently selected from R⁸⁰.

In certain embodiments, R⁶⁵, R⁶⁶, and R⁶⁷ are independently selected at each occurrence from the group consisting of hydrogen, cyano, haloalkyl, F, Cl, Br, I, heterocycle, —C(O)R³, —N(R′)—C(O)R³, —O—C(O)R³, and —O—S(O)₂R³, each of which is optionally substituted with 1, 2, or 3 substituents independently selected from R⁸⁰.

In certain embodiments, R⁶⁵, R⁶⁶, and R⁶⁷ are independently selected at each occurrence from the group consisting of hydrogen, C₂-C₄alkyl-cyano, haloalkyl, F, Cl, Br, I, heterocycle, C₂-C₄alkyl-C(O)R³, C₂-C₄alkyl-N(R′)—C(O)R³, and C₂-C₄alkyl-O—C(O)R³, each of which is optionally substituted with 1, 2, or 3 substituents independently selected from R⁸⁰.

In certain embodiments, R⁶⁵, R⁶⁶, and R^{6′} are independently selected at each occurrence from the group consisting of hydrogen, C₀-C₆alkyl-cyano, haloalkyl, F, and C₁.

In certain embodiments, R⁸⁰ is independently selected at each instance from the group consisting of alkyl, alkenyl, alkynyl, haloalkyl, —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.

In certain embodiments, R⁸⁰ is independently selected at each instance from the group consisting of alkyl, alkenyl, alkynyl, haloalkyl, —OR⁶, F, C₁, —NR⁶R⁷, heterocycle, heteroaryl, aryl, cyano, nitro, hydroxyl, azide, amide, —S(O)(NR⁶)R³, —NR⁸C(O)R³, —C(O)NR⁶R⁷, and —C(O)R³.

In certain embodiments, R⁸⁰ is independently selected at each instance from the group consisting of C₁-C₄alkyl, C₂-C₄alkenyl, C₂-C₄alkynyl, C₁-C₄haloalkyl, —OR⁶, F, C₁, —NR⁶R⁷, heterocycle, heteroaryl, aryl, cyano, nitro, hydroxyl, azide, amide, —S(O)(NR⁶)R³, —NR⁸C(O)R³, —C(O)NR⁶R⁷, and —C(O)R³.

In certain embodiments, R⁸⁰ is independently selected at each instance from the group consisting of C₁-C₄alkyl, C₁-C₄haloalkyl, —OR⁶, F, C₁, —NR⁶R⁷, heterocycle, heteroaryl, aryl, cyano, nitro, hydroxyl, azide, amide, —S(O)(NR⁶)R³, —NR⁸C(O)R³, —C(O)NR⁶R⁷, and —C(O)R³.

In certain embodiments, R⁸⁰ is independently selected at each instance from the group consisting of C₁-C₄alkyl, C₁-C₄haloalkyl, —OR⁶, F, C₁, —NR⁶R⁷, heterocycle, heteroaryl, and aryl.

### Embodiments of R¹

In certain embodiments R¹ is hydrogen.

In certain embodiments R¹ is

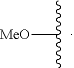 [SMILES: *OC]

In certain embodiments R¹ is

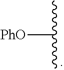 [SMILES: *Oc1ccccc1]

In certain embodiments R¹ is

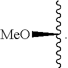

In certain embodiments R¹ is

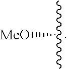

In certain embodiments R¹ is

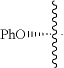

In certain embodiments R¹ is

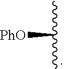

In certain embodiments R¹ is C₀-C₆alkyl-cyano optionally substituted with 1, 2, 3, or 4 substituents.

In certain embodiments R¹ is alkyl optionally substituted with 1, 2, 3, or 4 substituents.

In certain embodiments R¹ is alkenyl optionally substituted with 1, 2, 3, or 4 substituents.

In certain embodiments R¹ is alkynyl optionally substituted with 1, 2, 3, or 4 substituents.

In certain embodiments R¹ is haloalkyl optionally substituted with 1, 2, 3, or 4 substituents.

In certain embodiments R¹ is F.

In certain embodiments R¹ is C₁.

In certain embodiments R¹ is Br.

In certain embodiments R¹ is aryl optionally substituted with 1, 2, 3, or 4 substituents.

In certain embodiments R¹ is arylalkyl optionally substituted with 1, 2, 3, or 4 substituents.

In certain embodiments R¹ is heteroaryl optionally substituted with 1, 2, 3, or 4 substituents.

In certain embodiments R¹ is heteroarylalkyl optionally substituted with 1, 2, 3, or 4 substituents.

In certain embodiments R¹ is heterocycle optionally substituted with 1, 2, 3, or 4 substituents.

In certain embodiments R¹ is heterocycloalkyl optionally substituted with 1, 2, 3, or 4 substituents.

In certain embodiments R¹ is haloalkoxy optionally substituted with 1, 2, 3, or 4 substituents.

In certain embodiments R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R

In certain embodiments R³ is hydrogen or alkyl.

In certain embodiments R³ is hydrogen.

In certain embodiments R³ is alkyl.

In certain embodiments R³ is haloalkyl.

In certain embodiments R³ is arylalkyl.

In certain embodiments R³ is heteroarylalkyl.

In certain embodiments R³ is alkenyl.

In certain embodiments R³ is alkynyl.

In certain embodiments R³ is aryl.

In certain embodiments R³ is phenyl.

In certain embodiments R³ is heteroaryl.

In certain embodiments R³ is pyridine.

In certain embodiments R³ is heterocycle.

In certain embodiments R³ is —OR⁸.

In certain embodiments R³ is —OH.

In certain embodiments R³ is —NR⁸R⁹.

In certain embodiments R³ is —NH₂.

### Embodiments of R⁵

In certain embodiments R⁵ is hydrogen.

In certain embodiments R⁵ is

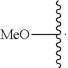 [SMILES: *OC]

In certain embodiments R⁵ is

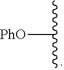 [SMILES: *Oc1ccccc1]

In certain embodiments R⁵ is

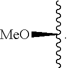

In certain embodiments R⁵ is

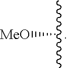

In certain embodiments R⁵ is

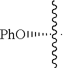

In certain embodiments R⁵ is

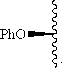

In certain embodiments R⁵ is C₀-C₆alkyl-cyano optionally substituted with 1, 2, 3, or 4 substituents.

In certain embodiments R⁵ is alkyl optionally substituted with 1, 2, 3, or 4 substituents.

In certain embodiments R⁵ is alkenyl optionally substituted with 1, 2, 3, or 4 substituents.

In certain embodiments R⁵ is alkynyl optionally substituted with 1, 2, 3, or 4 substituents.

In certain embodiments R⁵ is haloalkyl optionally substituted with 1, 2, 3, or 4 substituents.

In certain embodiments R⁵ is F.

In certain embodiments R⁵ is Cl.

In certain embodiments R⁵ is Br.

In certain embodiments R⁵ is aryl optionally substituted with 1, 2, 3, or 4 substituents.

In certain embodiments R⁵ is arylalkyl optionally substituted with 1, 2, 3, or 4 substituents.

In certain embodiments R⁵ is heteroaryl optionally substituted with 1, 2, 3, or 4 substituents.

In certain embodiments R⁵ is heteroarylalkyl optionally substituted with 1, 2, 3, or 4 substituents.

In certain embodiments R⁵ is heterocycle optionally substituted with 1, 2, 3, or 4 substituents.

In certain embodiments R⁵ is heterocycloalkyl optionally substituted with 1, 2, 3, or 4 substituents.

In certain embodiments R⁵ is haloalkoxy optionally substituted with 1, 2, 3, or 4 substituents.

In certain embodiments R⁵ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R′)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R⁶ and R⁷

In certain embodiments R⁶ and R⁷ are independently selected from hydrogen and alkyl.

In certain embodiments R⁶ is C(O)Me and R⁷ is H.

In certain embodiments R⁶ is hydrogen.

In certain embodiments R⁶ is alkyl.

In certain embodiments R⁶ is methyl.

In certain embodiments R⁶ is ethyl.

In certain embodiments R⁶ is arylalkyl.

In certain embodiments R⁶ is benzyl.

In certain embodiments R⁶ is heteroarylalkyl.

In certain embodiments R⁶ is alkenyl.

In certain embodiments R⁶ is alkynyl.

In certain embodiments R⁶ is aryl.

In certain embodiments R⁶ is phenyl.

In certain embodiments R⁶ is arylalkyl.

In certain embodiments R⁶ is haloalkyl.

In certain embodiments R⁶ is trifluoromethyl.

In certain embodiments R⁶ is heteroaryl.

In certain embodiments R⁶ is heterocycle.

In certain embodiments R⁶ is -alkyl-OR⁸.

In certain embodiments R⁶ is -alkyl-NR⁸R⁹.

In certain embodiments R⁶ is C(O)R³.

In certain embodiments R⁶ is C(O)Me.

In certain embodiments R⁶ is S(O)R³.

In certain embodiments R⁶ is C(S)R³.

In certain embodiments R⁶ is S(O)₂R³.

In certain embodiments R⁷ is hydrogen.

In certain embodiments R⁷ is alkyl.

In certain embodiments R⁷ is methyl.

In certain embodiments R⁷ is ethyl.

In certain embodiments R⁷ is arylalkyl.

In certain embodiments R⁷ is benzyl.

In certain embodiments R⁷ is heteroarylalkyl.

In certain embodiments R⁷ is alkenyl.

In certain embodiments R⁷ is alkynyl.

In certain embodiments R⁷ is aryl.

In certain embodiments R⁷ is phenyl.

In certain embodiments R⁷ is arylalkyl.

In certain embodiments R⁷ is haloalkyl.

In certain embodiments R⁷ is trifluoromethyl.

In certain embodiments R⁷ is heteroaryl.

In certain embodiments R⁷ is heterocycle.

In certain embodiments R⁷ is -alkyl-OR⁸.

In certain embodiments R⁷ is -alkyl-NR⁸R⁹.

In certain embodiments R⁷ is C(O)R³.

In certain embodiments R⁷ is C(O)Me.

In certain embodiments R⁷ is S(O)R³.

In certain embodiments R⁷ is C(S)R³.

In certain embodiments R⁷ is S(O)₂R³.

### Embodiments of R⁸ and R⁹

In certain embodiments R⁸ and R⁹ are independently selected from hydrogen and alkyl.

In certain embodiments R⁸ is hydrogen.

In certain embodiments R⁸ is alkyl.

In certain embodiments R⁸ is methyl.

In certain embodiments R⁸ is ethyl.

In certain embodiments R⁸ is arylalkyl.

In certain embodiments R⁸ is benzyl.

In certain embodiments R⁸ is heteroarylalkyl.

In certain embodiments R⁸ is alkenyl.

In certain embodiments R⁸ is alkynyl.

In certain embodiments R⁸ is aryl.

In certain embodiments R⁸ is phenyl.

In certain embodiments R⁸ is arylalkyl.

In certain embodiments R⁸ is haloalkyl.

In certain embodiments R⁸ is trifluoromethyl.

In certain embodiments R⁸ is heteroaryl.

In certain embodiments R⁸ is heterocycle.

In certain embodiments R⁹ is hydrogen.

In certain embodiments R⁹ is alkyl.

In certain embodiments R⁹ is methyl.

In certain embodiments R⁹ is ethyl.

In certain embodiments R⁹ is arylalkyl.

In certain embodiments R⁹ is benzyl.

In certain embodiments R⁹ is heteroarylalkyl.

In certain embodiments R⁹ is alkenyl.

In certain embodiments R⁹ is alkynyl.

In certain embodiments R⁹ is aryl.

In certain embodiments R⁹ is phenyl.

In certain embodiments R⁹ is arylalkyl.

In certain embodiments R⁹ is haloalkyl.

In certain embodiments R⁹ is trifluoromethyl.

In certain embodiments R⁹ is heteroaryl.

In certain embodiments R⁹ is heterocycle.

### Embodiments of R¹⁰

In certain embodiments R¹⁰ is hydrogen.

In certain embodiments R¹⁰ is alkyl.

In certain embodiments R¹⁰ is haloalkyl.

In certain embodiments R¹⁰ is C(O)R³.

### Embodiments of R¹¹, R¹², R¹³, R¹⁴, R¹⁵, R¹⁶, R¹⁷, R¹⁸, R¹⁹, and R²⁰

In certain embodiments R¹¹ is bond.

In certain embodiments R¹¹ is alkyl.

In certain embodiments R¹¹ is C(O).

In certain embodiments R¹¹ is —C(O)O—.

In certain embodiments R¹¹ is —C(O)NR⁶—.

In certain embodiments R¹¹ is —O—.

In certain embodiments R¹¹ is —NR⁶—.

In certain embodiments R¹¹ is a divalent residue of a natural amino acid.

In certain embodiments R¹¹ is a divalent residue of an unnatural amino acid.

In certain embodiments R¹¹ is —CH₂CH₂—[O—(CH₂)₂]ₙ—O—.

In certain embodiments R¹¹ is —CH₂CH₂—[O—(CH₂)₂]ₙ—N₆—.

In certain embodiments R¹¹ is —CH₂CH₂—[O—(CH₂)₂]ₙ—.

In certain embodiments R¹² is bond.

In certain embodiments R¹² is alkyl.

In certain embodiments R¹² is a divalent residue of a natural amino acid.

In certain embodiments R¹² is a divalent residue of an unnatural amino acid.

In certain embodiments R¹³ is bond.

In certain embodiments R¹³ is alkyl.

In certain embodiments R¹³ is C(O).

In certain embodiments R¹³ is —C(O)O—.

In certain embodiments R¹³ is —C(O)NR⁶—.

In certain embodiments R¹³ is —O—.

In certain embodiments R¹³ is —NR⁶—.

In certain embodiments R¹³ is a divalent residue of a natural amino acid.

In certain embodiments R¹³ is a divalent residue of an unnatural amino acid.

In certain embodiments R¹³ is —CH₂CH₂—[O—(CH₂)₂]ₙ—O—.

In certain embodiments R¹³ is —CH₂CH₂—[O—(CH₂)₂]ₙ—N₆—.

In certain embodiments R¹³ is —CH₂CH₂—[O—(CH₂)₂]ₙ—.

In certain embodiments R¹⁴ is bond.

In certain embodiments R¹⁴ is alkyl.

In certain embodiments R¹⁴ is a divalent residue of a natural amino acid.

In certain embodiments R¹⁴ is a divalent residue of an unnatural amino acid.

In certain embodiments R¹⁵ is bond.

In certain embodiments R¹⁵ is alkyl.

In certain embodiments R¹⁵ is C(O).

In certain embodiments R¹⁵ is —C(O)O—.

In certain embodiments R¹⁵ is —C(O)NR⁶—.

In certain embodiments R¹⁵ is —O—.

In certain embodiments R¹⁵ is —NR⁶—.

In certain embodiments R¹⁵ is a divalent residue of a natural amino acid.

In certain embodiments R¹⁵ is a divalent residue of an unnatural amino acid.

In certain embodiments R¹⁵ is —CH₂CH₂—[O—(CH₂)₂]ₙ—O—.

In certain embodiments R¹⁵ is —CH₂CH₂—[O—(CH₂)₂]ₙ—N₆—.

In certain embodiments R¹⁵ is —CH₂CH₂—[O—(CH₂)₂]ₙ—.

In certain embodiments R¹⁶ is bond.

In certain embodiments R¹⁶ is alkyl.

In certain embodiments R¹⁶ is a divalent residue of a natural amino acid.

In certain embodiments R¹⁶ is a divalent residue of an unnatural amino acid.

In certain embodiments R¹⁷ is bond.

In certain embodiments R¹⁷ is alkyl.

In certain embodiments R¹⁷ is C(O).

In certain embodiments R¹⁷ is —C(O)O—.

In certain embodiments R¹⁷ is —C(O)NR⁶—.

In certain embodiments R¹⁷ is —O—.

In certain embodiments R¹⁷ is —NR⁶—.

In certain embodiments R¹⁷ is a divalent residue of a natural amino acid.

In certain embodiments R¹⁷ is a divalent residue of an unnatural amino acid.

In certain embodiments R¹⁷ is —CH₂CH₂—[O—(CH₂)₂]ₙ—O—.

In certain embodiments R¹⁷ is —CH₂CH₂—[O—(CH₂)₂]ₙ—NR⁶—.

In certain embodiments R¹⁷ is —CH₂CH₂—[O—(CH₂)₂]ₙ—.

In certain embodiments R¹⁸ is bond.

In certain embodiments R¹⁸ is alkyl.

In certain embodiments R¹⁸ is a divalent residue of a natural amino acid.

In certain embodiments R¹⁸ is a divalent residue of an unnatural amino acid.

In certain embodiments R¹⁹ is bond.

In certain embodiments R¹⁹ is alkyl.

In certain embodiments R¹⁹ is C(O).

In certain embodiments R¹⁹ is —C(O)O—.

In certain embodiments R¹⁹ is —C(O)NR⁶—.

In certain embodiments R¹⁹ is —O—.

In certain embodiments R¹⁹ is —NR⁶—.

In certain embodiments R¹⁹ is a divalent residue of a natural amino acid.

In certain embodiments R¹⁹ is a divalent residue of an unnatural amino acid.

In certain embodiments R¹⁹ is —CH₂CH₂—[O—(CH₂)₂]ₙ—O—.

In certain embodiments R¹⁹ is —CH₂CH₂—[O—(CH₂)₂]ₙ—NR⁶—.

In certain embodiments R¹⁹ is —CH₂CH₂—[O—(CH₂)₂]ₙ—.

In certain embodiments R²⁰ is bond.

In certain embodiments R²⁰ is alkyl.

In certain embodiments R²⁰ is a divalent residue of a natural amino acid.

In certain embodiments R²⁰ is a divalent residue of an unnatural amino acid.

In certain embodiments n is 0.

In certain embodiments n is 1.

In certain embodiments n is 2.

In certain embodiments n is 3.

In certain embodiments n is 4.

In certain embodiments n is 5.

In certain embodiments n is 6.

In certain embodiments n is 7.

In certain embodiments n is 8.

In certain embodiments n is 9.

In certain embodiments n is 10.

### Embodiments of R²¹

In certain embodiments R²¹ is hydrogen.

In certain embodiments R²¹ is alkyl.

In certain embodiments R²¹ is methyl.

In certain embodiments R²¹ is F.

In certain embodiments R²¹ is C₁.

In certain embodiments R²¹ is haloalkyl.

In certain embodiments R²¹ is C(O)R³.

In certain embodiments R²¹ is NH₂.

In certain embodiments R²¹ is OH.

### Embodiments of R²²

In certain embodiments R²² is alkyl.

In certain embodiments R²² is —C(O)N—.

In certain embodiments R²² is —NC(O)—.

In certain embodiments R²² is —N—.

In certain embodiments R²² is —C(R²¹)—.

In certain embodiments R²² is —P(O)O—.

In certain embodiments R²² is —P(O)—.

In certain embodiments R²² is —P(O)(NR⁶R⁷)N—.

In certain embodiments R²² is alkenyl.

In certain embodiments R²² is haloalkyl.

In certain embodiments R²² is aryl.

In certain embodiments R²² is heterocycle.

In certain embodiments R²² is heteroaryl.

### Embodiments of R³²

In certain embodiments R³² is alkyl.

In certain embodiments R³² is N⁺X⁻.

In certain embodiments R³² is —C—.

In certain embodiments R³² is alkenyl.

In certain embodiments R³² is haloalkyl.

In certain embodiments R³² is aryl.

In certain embodiments R³² is heterocycle.

In certain embodiments R³² is heteroaryl.

In certain embodiments X⁻ is Br⁻. or

In certain embodiments X⁻ is Cl⁻.

### Embodiments of R⁴²

In certain embodiments R⁴² is hydrogen.

In certain embodiments R⁴² is alkyl.

In certain embodiments R⁴² is alkyl-O-alkyl.

In certain embodiments R⁴² is C(O)R⁴³.

In certain embodiments R⁴² is C(O)Me.

### Embodiments of R⁴³

In certain embodiments R⁴³ is hydrogen.

In certain embodiments R⁴³ is alkyl.

In certain embodiments R⁴³ is haloalkyl.

### Embodiments of R⁶⁵, R⁶⁶, and R⁶⁷

In certain embodiments R⁶⁵, R⁶⁶, and R⁶⁷ are independently selected from hydrogen, halogen, and haloalkyl.

In certain embodiments R⁶⁵ is hydrogen.

In certain embodiments R⁶⁵ is alkyl.

In certain embodiments R⁶⁵ is methyl.

In certain embodiments R⁶⁵ is halogen.

In certain embodiments R⁶⁵ is F.

In certain embodiments R⁶⁵ is Cl.

In certain embodiments R⁶⁵ is haloalkyl.

In certain embodiments R⁶⁵ is CF₃.

In certain embodiments R⁶⁵ is OMe.

In certain embodiments R⁶⁵ is OH.

In certain embodiments R⁶⁵ is NH₂.

In certain embodiments R⁶⁵ is NHMe.

In certain embodiments R⁶⁵ is NMe₂.

In certain embodiments R⁶⁵ is heterocycle.

In certain embodiments R⁶⁶ is hydrogen.

In certain embodiments R⁶⁶ is alkyl.

In certain embodiments R⁶⁶ is methyl.

In certain embodiments R⁶⁶ is halogen.

In certain embodiments R⁶⁶ is F.

In certain embodiments R⁶⁶ is C₁.

In certain embodiments R⁶⁶ is haloalkyl.

In certain embodiments R⁶⁶ is CF₃.

In certain embodiments R⁶⁶ is OMe.

In certain embodiments R⁶⁶ is OH.

In certain embodiments R⁶⁶ is NH₂.

In certain embodiments R⁶⁶ is NHMe.

In certain embodiments R⁶⁶ is NMe₂.

In certain embodiments R⁶⁶ is heterocycle.

In certain embodiments R⁶⁷ is hydrogen.

In certain embodiments R⁶⁷ is alkyl.

In certain embodiments R⁶⁷ is methyl.

In certain embodiments R⁶⁷ is halogen.

In certain embodiments R⁶⁷ is F.

In certain embodiments R⁶⁷ is C₁.

In certain embodiments R⁶⁷ is haloalkyl.

In certain embodiments R⁶⁷ is CF₃.

In certain embodiments R⁶⁷ is OMe.

In certain embodiments R⁶⁷ is OH.

In certain embodiments R⁶⁷ is NH₂.

In certain embodiments R⁶⁷ is NHMe.

In certain embodiments R⁶⁷ is NMe₂.

In certain embodiments R⁶⁷ is heterocycle.

### Embodiments of R⁷⁵ and R⁷⁶

In certain embodiments R⁷⁵ and R⁷⁶ are independently selected from hydrogen, halogen, and haloalkyl.

In certain embodiments R⁷⁵ is hydrogen.

In certain embodiments R⁷⁵ is alkyl.

In certain embodiments R⁷⁵ is methyl.

In certain embodiments R⁷⁵ is halogen.

In certain embodiments R⁷⁵ is F.

In certain embodiments R⁷⁵ is C₁.

In certain embodiments R⁷⁵ is haloalkyl.

In certain embodiments R⁷⁵ is CF₃.

In certain embodiments R⁷⁵ is OMe.

In certain embodiments R⁷⁵ is OH.

In certain embodiments R⁷⁵ is NH₂.

In certain embodiments R⁷⁵ is NHMe.

In certain embodiments R⁷⁵ is NMe₂.

In certain embodiments R⁷⁵ is heterocycle.

In certain embodiments R⁷⁶ is hydrogen.

In certain embodiments R⁷⁶ is alkyl.

In certain embodiments R⁷⁶ is methyl.

In certain embodiments R⁷⁶ is halogen.

In certain embodiments R⁷⁶ is F.

In certain embodiments R⁷⁶ is Cl.

In certain embodiments R⁷⁶ is haloalkyl.

In certain embodiments R⁷⁶ is CF₃.

In certain embodiments R⁷⁶ is OMe.

In certain embodiments R⁷⁶ is OH.

In certain embodiments R⁷⁶ is NH₂.

In certain embodiments R⁷⁶ is NHMe.

In certain embodiments R⁷⁶ is NMe₂.

In certain embodiments R⁷⁶ is heterocycle.

### Embodiments of R⁸⁰

In certain embodiments R⁸⁰ is hydrogen.

In certain embodiments R⁸⁰ is alkyl.

In certain embodiments R⁸⁰ is methyl.

In certain embodiments R⁸⁰ is halogen.

In certain embodiments R⁸⁰ is F.

In certain embodiments R⁸⁰ is C₁.

In certain embodiments R⁸⁰ is haloalkyl.

In certain embodiments R⁸⁰ is CF₃.

In certain embodiments R⁸⁰ is OMe.

In certain embodiments R⁸⁰ is OH.

In certain embodiments R⁸⁰ is NH₂.

In certain embodiments R⁸⁰ is NHMe.

In certain embodiments R⁸⁰ is NMe₂.

In certain embodiments R⁸⁰ is —C(O)R³.

### Embodiments of R¹⁰²

In certain embodiments R¹⁰² is hydrogen.

In certain embodiments R¹⁰² is alkyl.

In certain embodiments R¹⁰² is methyl.

In certain embodiments R¹⁰² is halogen.

In certain embodiments R¹⁰² is F.

In certain embodiments R¹⁰² is C₁.

In certain embodiments R¹⁰² is haloalkyl.

In certain embodiments R¹⁰² is CF₃.

In certain embodiments R¹⁰² is OMe.

In certain embodiments R¹⁰² is OH.

In certain embodiments R¹⁰² is NH₂.

In certain embodiments R¹⁰² is NHMe.

In certain embodiments R¹⁰² is NMe₂.

In certain embodiments R¹⁰² is —C(O)R³.

### Embodiments of n, m, and p

In certain embodiments nn is 0.

In certain embodiments nn is 1.

In certain embodiments nn is 2.

In certain embodiments nn is 3.

In certain embodiments m is 0.

In certain embodiments m is 1.

In certain embodiments m is 2.

In certain embodiments m is 3.

In certain embodiments p is 0.

In certain embodiments p is 1.

In certain embodiments p is 2.

In certain embodiments p is 3.

### Embodiments of ASGPR Binder

B1. In certain embodiments a compound of Formula I, Formula II, or Formula III is provided wherein, the ASGPR Binding Ligand is selected from:

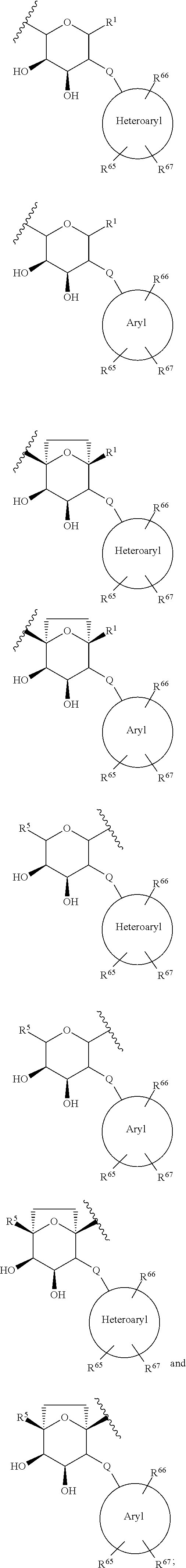

wherein
Q is —O— or —N(R¹⁰)—;
R¹ and R⁵ are independently hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R′)—C(S)R³, C₀-C₆alkyl-N(R′)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³;
in certain embodiments R¹ is hydrogen;
R² is hydrogen, alkyl, or C(O)R³;
R³ at each occurrence is independently selected from the group consisting of hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from the group consisting of hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is hydrogen, alkyl, haloalkyl, arylalkyl, heteroarylalkyl, alkenyl, alkynyl, aryl, heteroaryl, heterocycle, C(O)R³, S(O)R³, C(S)R³, or S(O)₂R³;
in certain embodiments R¹⁰ is hydrogen;
R⁶⁵, R⁶⁶, and R⁶⁷ are independently selected at each occurrence from the group consisting of hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R′)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰;
R⁸⁰ is independently selected at each instance from the group consisting of alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.
B2. The compound of embodiment B1, wherein the compound is selected from

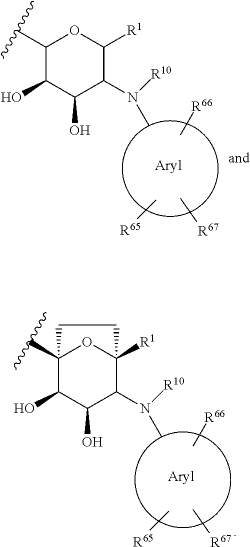

B3. The compound of embodiment B1, wherein the compound is selected from

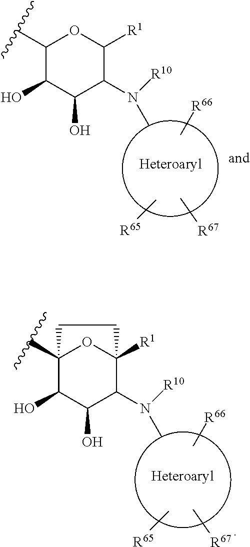

B4. The compound of any one of embodiments B1-B3, wherein R⁶⁵ is not substituted with an optional substituent.
B5. The compound of any one of embodiments B1-B3, wherein R⁶⁵ is substituted with 1 optional substituent.
B6. The compound of any one of embodiments B1-B3, wherein R⁶⁵ is substituted with 2 optional substituents.
B7. The compound of any one of embodiments B1-B3, wherein R⁶⁵ is substituted with 3 optional substituents.
B8. The compound of any one of embodiments B1-B3, wherein R⁶⁵ is hydrogen.
B9. The compound of any one of embodiments B1-B3, wherein R⁶⁵ is —CF₃.
B10. The compound of any one of embodiments B1-B3, wherein R⁶⁵ is cyano.
B11. The compound of any one of embodiments B1-B3, wherein R⁶⁵ is F.
B12. The compound of any one of embodiments B1-B3, wherein R⁶⁵ is C₁.
B13. The compound of any one of embodiments B1-B3, wherein R⁶⁵ is Br.
B14. The compound of any one of embodiments B1-B7, wherein R⁶⁵ is haloalkyl.
B15. The compound of any one of embodiments B1-B7, wherein R⁶⁵ is heterocycle.
B16. The compound of any one of embodiments B1-B7, wherein R⁶⁵ is haloalkoxy.
B17. The compound of any one of embodiments B1-B7, wherein R⁶⁵ is C₀-C₆alkyl-OR⁶.
B18. The compound of any one of embodiments B1-B7, wherein R⁶⁵ is C₀-C₆alkyl-SR⁶.
B19. The compound of any one of embodiments B1-B7, wherein R⁶⁵ is C₀-C₆alkyl-NR⁶R⁷.
B20. The compound of any one of embodiments B1-B7, wherein R⁶⁵ is C₀-C₆alkyl-C(O)R³.
B21. The compound of any one of embodiments B1-B7, wherein R⁶⁵ is C₀-C₆alkyl-S(O)R³.
B22. The compound of any one of embodiments B1-B7, wherein R⁶⁵ is C₀-C₆alkyl-C(S)R³.
B23. The compound of any one of embodiments B1-B7, wherein R⁶⁵ is C₀-C₆alkyl-S(O)₂R³.
B24. The compound of any one of embodiments B1-B7, wherein R⁶⁵ is C₀-C₆alkyl-N(R′)—C(O)R³.
B25. The compound of any one of embodiments B1-B7, wherein R⁶⁵ is C₀-C₆alkyl-N(R′)—S(O)R³.
B26. The compound of any one of embodiments B1-B7, wherein R⁶⁵ is C₀-C₆alkyl-N(R′)—C(S)R³.
B27. The compound of any one of embodiments B1-B7, wherein R⁶⁵ is C₀-C₆alkyl-N(R′)—S(O)₂R³.
B28. The compound of any one of embodiments B1-B7, wherein R⁶⁵ is C₀-C₆alkylN₃.
B29. The compound of any one of embodiments B1-B7, wherein R⁶⁵ is C₀-C₆alkyl-cyano.
B30. The compound of any one of embodiments B17-B29, wherein C₀-C₆alkyl is C₀-alkyl (i.e. bond).
B31. The compound of any one of embodiments B17-B29, wherein C₀-C₆alkyl is C₁-alkyl.
B32. The compound of any one of embodiments B17-B29, wherein C₀-C₆alkyl is C₂-alkyl.
B33. The compound of any one of embodiments B1-B7, wherein R⁶⁵ is —N═S(O)(R³)₂.
B34. The compound of any one of embodiments B1-B7, wherein R⁶⁵ is heterocycloalkyl.
B35. The compound of any one of embodiments B1-B7, wherein R⁶⁵ is aryl.
B36. The compound of any one of embodiments B1-B35, wherein R⁶⁶ is not substituted with an optional substituent.
B37. The compound of any one of embodiments B1-B35, wherein R⁶⁶ is substituted with 1 optional substituent.
B38. The compound of any one of embodiments B1-B35, wherein R⁶⁶ is substituted with 2 optional substituents.
B39. The compound of any one of embodiments B1-B35, wherein R⁶⁶ is substituted with 3 optional substituents.
B40. The compound of any one of embodiments B1-B35, wherein R⁶⁶ is hydrogen.
B41. The compound of any one of embodiments B1-B35, wherein R⁶⁶ is —CF₃.
B42. The compound of any one of embodiments B1-B35, wherein R⁶⁶ is cyano.
B43. The compound of any one of embodiments B1-B35, wherein R⁶⁶ is F.
B44. The compound of any one of embodiments B1-B35, wherein R⁶⁶ is C₁.
B45. The compound of any one of embodiments B1-B35, wherein R⁶⁶ is Br.
B46. The compound of any one of embodiments B1-B37, wherein R⁶⁶ is haloalkyl.
B47. The compound of any one of embodiments B1-B37, wherein R⁶⁶ is heterocycle.
B48. The compound of any one of embodiments B1-B37, wherein R⁶⁶ is haloalkoxy.
B49. The compound of any one of embodiments B1-B37, wherein R⁶⁶ is C₀-C₆alkyl-OR⁶.
B50. The compound of any one of embodiments B1-B37, wherein R⁶⁶ is C₀-C₆alkyl-SR⁶.
B51. The compound of any one of embodiments B1-B37, wherein R⁶⁶ is C₀-C₆alkyl-NR⁶R⁷.
B52. The compound of any one of embodiments B1-B37, wherein R⁶⁶ is C₀-C₆alkyl-C(O)R³.
B53. The compound of any one of embodiments B1-B37, wherein R⁶⁶ is C₀-C₆alkyl-S(O)R³.
B54. The compound of any one of embodiments B1-B37, wherein R⁶⁶ is C₀-C₆alkyl-C(S)R³.
B55. The compound of any one of embodiments B1-B37, wherein R⁶⁶ is C₀-C₆alkyl-S(O)₂R³.
B56. The compound of any one of embodiments B1-B37, wherein R⁶⁶ is C₀-C₆alkyl-N(R⁸)—C(O)R³.
B57. The compound of any one of embodiments B1-B37, wherein R⁶⁶ is C₀-C₆alkyl-N(R⁸)—S(O)R³.
B58. The compound of any one of embodiments B1-B37, wherein R⁶⁶ is C₀-C₆alkyl-N(R⁸)—C(S)R³.
B59. The compound of any one of embodiments B1-B37, wherein R⁶⁶ is C₀-C₆alkyl-N(R⁸)—S(O)₂R³.
B60. The compound of any one of embodiments B1-B37, wherein R⁶⁶ is C₀-C₆alkylN₃.
B61. The compound of any one of embodiments B1-B37, wherein R⁶⁶ is C₀-C₆alkyl-cyano.
B62. The compound of any one of embodiments B49-B61, wherein C₀-C₆alkyl is C₀-alkyl (i.e. bond).
B63. The compound of any one of embodiments B49-B61, wherein C₀-C₆alkyl is C₁-alkyl.
B64. The compound of any one of embodiments B49-B61, wherein C₀-C₆alkyl is C₂-alkyl.
B65. The compound of any one of embodiments B1-B35, wherein R⁶⁶ is —N═S(O)(R³)₂.
B66. The compound of any one of embodiments B1-B35, wherein R⁶⁶ is heterocycloalkyl.
B67. The compound of any one of embodiments B1-B35, wherein R⁶⁶ is aryl.
B68. The compound of any one of embodiments B1-B67, wherein R⁶⁷ is not substituted with an optional substituent.
B69. The compound of any one of embodiments B1-B67, wherein R⁶⁷ is substituted with 1 optional substituent.
B70. The compound of any one of embodiments B1-B67, wherein R⁶⁷ is substituted with 2 optional substituents.
B71. The compound of any one of embodiments B1-B67, wherein R⁶⁷ is substituted with 3 optional substituents.
B72. The compound of any one of embodiments B1-B67, wherein R⁶⁷ is hydrogen.
B73. The compound of any one of embodiments B1-B67, wherein R⁶⁷ is —CF₃.
B74. The compound of any one of embodiments B1-B67, wherein R⁶⁷ is cyano.
B75. The compound of any one of embodiments B1-B67, wherein R⁶⁷ is F.
B76. The compound of any one of embodiments B1-B67, wherein R⁶⁷ is C₁.
B77. The compound of any one of embodiments B1-B67, wherein R⁶⁷ is Br.
B78. The compound of any one of embodiments B1-B67, wherein R⁶⁷ is haloalkyl.
B79. The compound of any one of embodiments B1-B65, wherein R⁶⁷ is heterocycle.
B80. The compound of any one of embodiments B1-B67, wherein R⁶⁷ is haloalkoxy.
B81. The compound of any one of embodiments B1-B67, wherein R⁶⁷ is C₀-C₆alkyl-OR⁶.
B82. The compound of any one of embodiments B1-B67, wherein R⁶⁷ is C₀-C₆alkyl-SR⁶.
B83. The compound of any one of embodiments B1-B67, wherein R⁶⁷ is C₀-C₆alkyl-NR⁶R⁷.
B84. The compound of any one of embodiments B1-B67, wherein R⁶⁷ is C₀-C₆alkyl-C(O)R³.
B85. The compound of any one of embodiments B1-B67, wherein R⁶⁷ is C₀-C₆alkyl-S(O)R³.
B86. The compound of any one of embodiments B1-B67, wherein R⁶⁷ is C₀-C₆alkyl-C(S)R³.
B87. The compound of any one of embodiments B1-B67, wherein R⁶⁷ is C₀-C₆alkyl-S(O)₂R³.
B88. The compound of any one of embodiments B1-B67, wherein R⁶⁷ is C₀-C₆alkyl-N(R⁸)—C(O)R³.
B89. The compound of any one of embodiments B1-B67, wherein R⁶⁷ is C₀-C₆alkyl-N(R⁸)—S(O)R³.
B90. The compound of any one of embodiments B1-B67, wherein R⁶⁷ is C₀-C₆alkyl-N(R⁸)—C(S)R³.
B91. The compound of any one of embodiments B1-B67, wherein R⁶⁷ is C₀-C₆alkyl-N(R⁸)—S(O)₂R³.
B92. The compound of any one of embodiments B1-B67, wherein R⁶⁷ is C₀-C₆alkylN₃.
B93. The compound of any one of embodiments B1-B67, wherein R⁶⁷ is C₀-C₆alkyl-cyano.
B94. The compound of any one of embodiments B81-B93, wherein C₀-C₆alkyl is C₀-alkyl (i.e. bond).
B95. The compound of any one of embodiments B81-B93, wherein C₀-C₆alkyl is C₁-alkyl.
B96. The compound of any one of embodiments B81-B93, wherein C₀-C₆alkyl is C₂-alkyl.
B97. The compound of any one of embodiments B1-B67, wherein R⁶⁷ is —N═S(O)(R³)₂.
B98. The compound of any one of embodiments B1-B67, wherein R⁶⁷ is heterocycloalkyl.
B99. The compound of any one of embodiments B1-B67, wherein R⁶⁷ is aryl.
B100. The compound of embodiment B3, wherein heteroaryl is

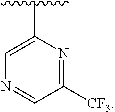 [SMILES: *c1cncc(C(F)(F)F)n1]

B101. The compound of embodiment B3, wherein heteroaryl is

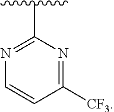 [SMILES: *c1nccc(C(F)(F)F)n1]

B102. The compound of embodiment B3, wherein heteroaryl is,

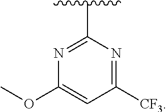 [SMILES: *c1nc(OC)cc(C(F)(F)F)n1]

B103. The compound of embodiment B3, wherein heteroaryl is

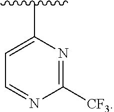 [SMILES: *c1ccnc(C(F)(F)F)n1]

B104. The compound of embodiment B3, wherein heteroaryl is

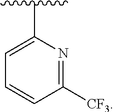 [SMILES: *c1cccc(C(F)(F)F)n1]

B105. The compound of embodiment B3, wherein heteroaryl is

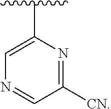 [SMILES: *c1cncc(C#N)n1]

B106. The compound of embodiment B3, wherein heteroaryl is

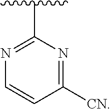 [SMILES: *c1nccc(C#N)n1]

B107. The compound of embodiment B3, wherein heteroaryl is

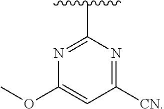 [SMILES: *c1nc(C#N)cc(OC)n1]

B108. The compound of embodiment B3, wherein heteroaryl is

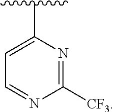 [SMILES: *c1ccnc(C(F)(F)F)n1]

B109. The compound of embodiment B3, wherein heteroaryl is

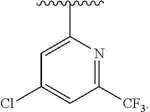 [SMILES: *c1cc(Cl)cc(C(F)(F)F)n1]

B110. The compound of embodiment B3, wherein heteroaryl is

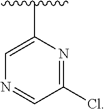 [SMILES: *c1cncc(Cl)n1]

B111. The compound of embodiment B3, wherein heteroaryl is

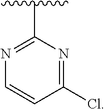 [SMILES: *c1nccc(Cl)n1]

B112. The compound of embodiment B3, wherein heteroaryl is

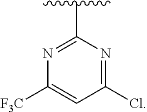 [SMILES: *c1nc(Cl)cc(C(F)(F)F)n1]

B113. The compound of embodiment B3, wherein heteroaryl is

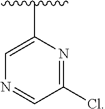 [SMILES: *c1cncc(Cl)n1]

B114. The compound of embodiment B3, wherein heteroaryl is

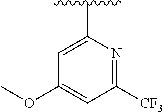 [SMILES: *c1cc(OC)cc(C(F)(F)F)n1]

B115. The compound of embodiment B3, wherein heteroaryl is

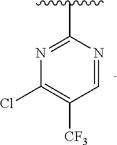 [SMILES: *c1ncc(C(F)(F)F)c(Cl)n1]

B116. The compound of embodiment B3, wherein heteroaryl is

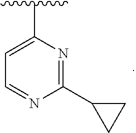 [SMILES: *c1ccnc(C2CC2)n1]

B117. The compound of embodiment B3, wherein heteroaryl is

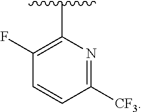 [SMILES: *c1nc(C(F)(F)F)ccc1F]

B118. The compound of embodiment B3, wherein heteroaryl is

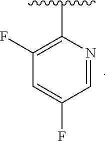 [SMILES: *c1ncc(F)cc1F]

B119. The compound of embodiment B3, wherein heteroaryl is

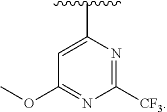 [SMILES: *c1cc(OC)nc(C(F)(F)F)n1]

B120. The compound of embodiment B3, wherein heteroaryl is

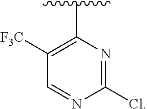 [SMILES: *c1nc(Cl)ncc1C(F)(F)F]

B121. The compound of embodiment B3, wherein heteroaryl is

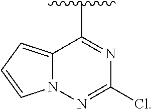 [SMILES: *c1nc(Cl)nn2cccc12]

B122. The compound of any one of embodiments B1-B121, wherein R¹⁰ is hydrogen.
B123. The compound of any one of embodiments B1-B121, wherein R¹⁰ is alkyl.
B124. The compound of any one of embodiments B1-B121, wherein R¹⁰ is methyl.
B125. The compound of any one of embodiments B1-B121, wherein R¹⁰ is deuterium.
B126. The compound of any one of embodiments B1-B121, wherein R¹⁰ is C(O)R³.
B127. The compound of any one of embodiments B1-B121, wherein R¹⁰ is arylalkyl.
B128. The compound of any one of embodiments B1-B127, wherein R¹ is hydrogen.
B129. The compound of any one of embodiments B1-B127, wherein R¹ is hydrogen.
B130. The compound of any one of embodiments B1-B127, wherein R¹ is aryl.
B131. The compound of any one of embodiments B1-B127, wherein R¹ is phenyl.
B132. The compound of any one of embodiments B1-B127, wherein R¹ is C(O)R³.
B133. The compound of any one of embodiments B1-B127, wherein R¹ is arylalkyl.
B134. The compound of any one of embodiments B1-B127, wherein R¹ is alkyl.
B135. The compound of any one of embodiments B1-B127, wherein R¹ is haloalkyl.
B136. The compound of any one of embodiments B1-B127, wherein R¹ is methyl.
B137. The compound of any one of embodiments B1-B127, wherein R¹ is C₀-C₆alkyl-cyano.
B138. The compound of any one of embodiments B1-B127, wherein R¹ is C₀-C₆alkyl-OR⁶
B139. The compound of any one of embodiments B1-B127, wherein R¹ is C₀-C₆alkyl-SR⁶
B140. The compound of any one of embodiments B1-B127, wherein R¹ is C₀-C₆alkyl-NR⁶R⁷
B141. The compound of any one of embodiments B1-B127, wherein R¹ is C₀-C₆alkyl-C(O)R³
B142. The compound of any one of embodiments B1-B127, wherein R¹ is C₀-C₆alkyl-S(O)R³
B143. The compound of any one of embodiments B1-B127, wherein R¹ is C₀-C₆alkyl-S(O)₂R³
B144. The compound of any one of embodiments B1-B127, wherein R¹ is C₀-C₆alkyl-N(R⁸)—C(O)R³
B145. The compound of any one of embodiments B1-B127, wherein R¹ is C₀-C₆alkyl-N(R⁸)—S(O)R³
B146. The compound of any one of embodiments B1-B127, wherein R¹ is C₀-C₆alkyl-N(R⁸)—S(O)₂R³
B147. The compound of any one of embodiments B1-B127, wherein R¹ is C₀-C₆alkyl-O—C(O)R³
B148. The compound of any one of embodiments B137-B147, wherein C₀-C₆alkyl is C₀-alkyl (i.e. bond).
B149. The compound of any one of embodiments B137-B147, wherein C₀-C₆alkyl is C₁-alkyl.
B150. The compound of any one of embodiments B137-B147, wherein C₀-C₆alkyl is C₂-alkyl.

In certain embodiments R⁶⁵, R⁶⁶, or R⁶⁷ is aryl.

In certain embodiments R⁶⁵, R⁶⁶, or R⁶⁷ is heteroaryl.

In certain embodiments R⁶⁵, R⁶⁶, or R⁶⁷ is heterocycle.

### Additional Embodiments

1. A compound of Formula:

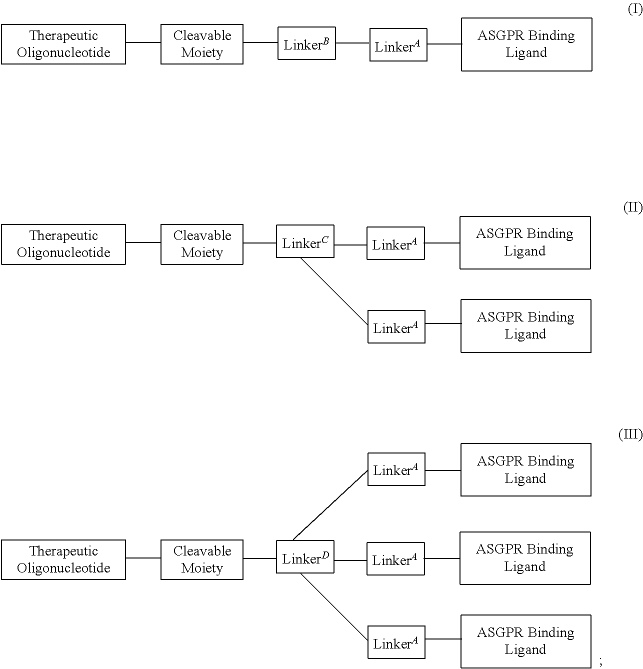

or a pharmaceutically acceptable salt thereof;
wherein:

ASGPR Binding Ligand is a compound selected from:

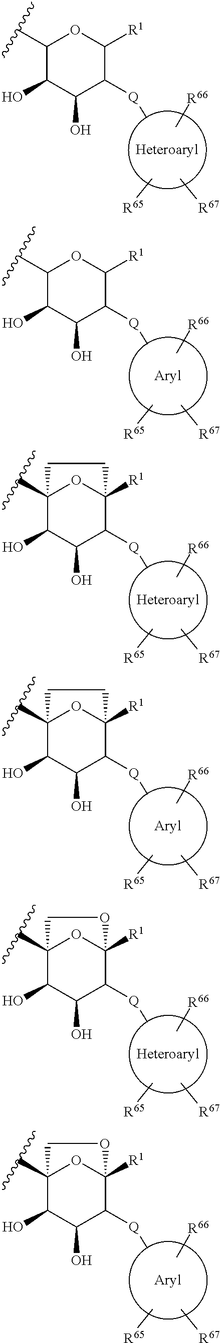

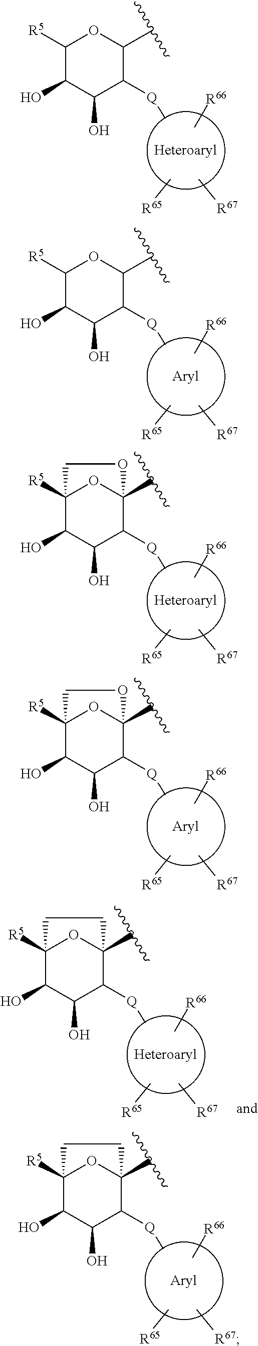

Q is —O— or —N(R¹⁰)—;
R¹ and R⁵ are independently hydrogen, alkyl, alkenyl, alkynyl, haloalkyl, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R⁸)—C(S)R³, C₀-C₆alkyl-N(R⁸)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³;
R³ at each occurrence is independently selected from the group consisting of hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from the group consisting of hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is hydrogen, alkyl, haloalkyl, arylalkyl, heteroarylalkyl, alkenyl, alkynyl, aryl, heteroaryl, heterocycle, C(O)R³, S(O)R³, C(S)R³, or S(O)₂R³;
R⁶⁵, R⁶⁶, and R⁶⁷ are independently selected at each occurrence from the group consisting of hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰;
R⁸⁰ is independently selected at each instance from the group consisting of alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.

Cleavable Moiety is a bond,

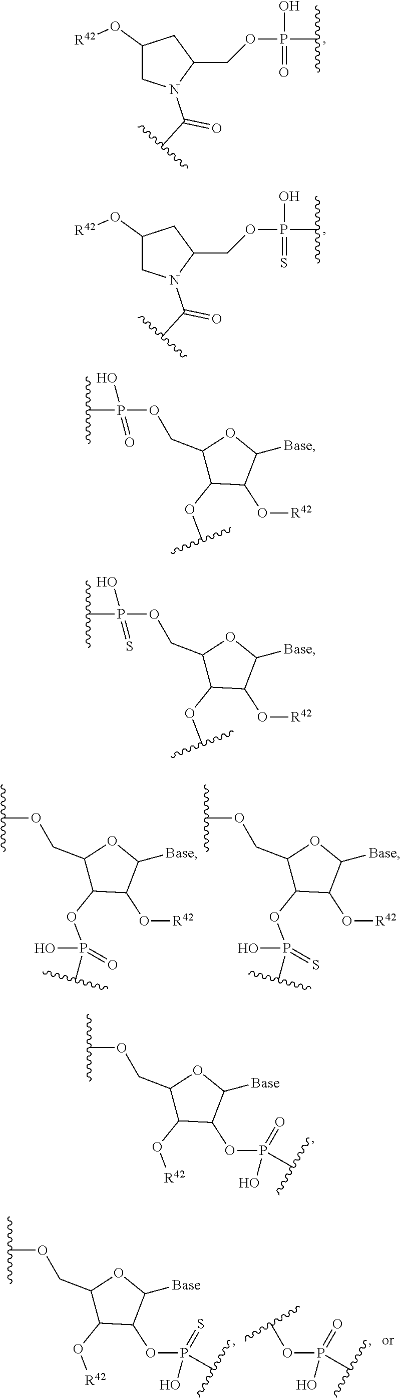

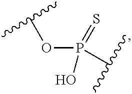 [SMILES: *OP(*)(O)=S]

wherein the Therapeutic Oligonucleotide is attached to the —P—, or Cleavable Moiety is

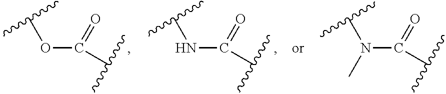

wherein the Therapeutic Oligonucleotide is bound to the —C—;
R⁴² is hydrogen, alkyl, alkyl-O-alkyl, or C(O)R⁴³.
R⁴³ is selected from the group consisting of hydrogen, alkyl, and haloalkyl;

Base is a heterocycle comprising two, three, or four nitrogen atoms, wherein the heterocycle is substituted with an oxo, amino, or carbamoyl and optionally substituted with 1, 2, or 3 substituents independently selected from alkyl, haloalkyl, and halogen;

Therapeutic Oligonucleotide is an antisense oligonucleotide, siRNA, shRNA, DNA aptamer, RNA aptamer, miRNA, miRNA mimic, antimiRNA, DNA decoy, RNA decoy, or CpG oligonucleotide;

Linker^{A} and Linker^{B} are independently selected from:

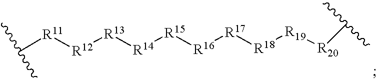

Linker^{C} is selected from:

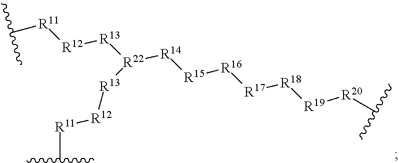

Linker^{D} is selected from:

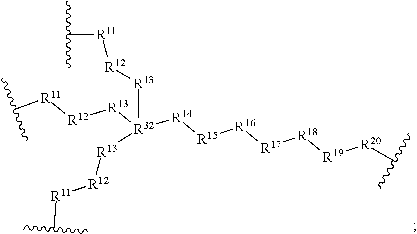

R¹¹, R¹², R¹³, R¹⁴, R¹⁵, R¹⁶, R¹⁷, R⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, and heterocycle;
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹; and
X⁻ is an anionic group.
2. The compound of embodiment 1, wherein R¹ is hydrogen.
3. The compound of embodiment 1 or embodiment 2, wherein the compound is of formula:

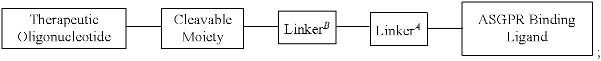

or a pharmaceutically acceptable salt thereof.
4. The compound of embodiment 1 or embodiment 2, wherein the compound is of formula:

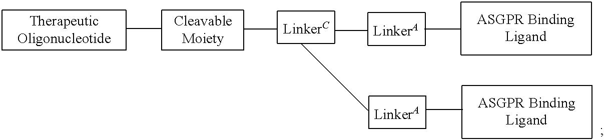

or a pharmaceutically acceptable salt thereof.
5. The compound of embodiment 1 or embodiment 2, wherein the compound is of formula:

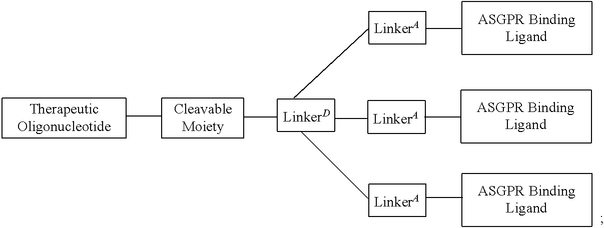

or a pharmaceutically acceptable salt thereof.
6. The compound of any one of embodiments 1-5, wherein the ASGPR Binding Ligand is

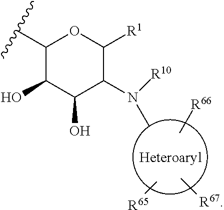

7. The compound of any one of embodiments 1-5, wherein the ASGPR Binding Ligand is

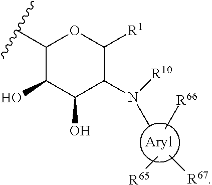

8. The compound of any one of embodiments 1-5, wherein the ASGPR Binding Ligand is

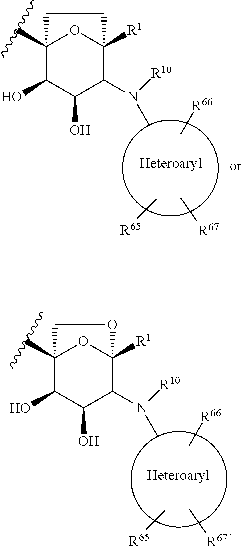

9. The compound of any one of embodiments 1-5, wherein the ASGPR Binding Ligand is

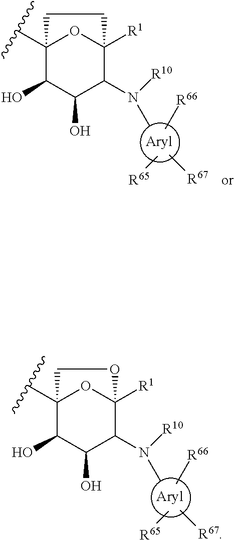

10. The compound of any one of embodiments 1-5, wherein the ASGPR Binding Ligand is

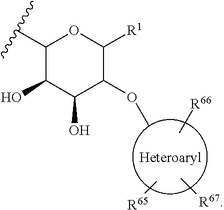

11. The compound of any one of embodiments 1-5, wherein the ASGPR Binding Ligand is

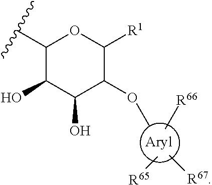

12. The compound of any one of embodiments 1-5, wherein the ASGPR Binding Ligand is

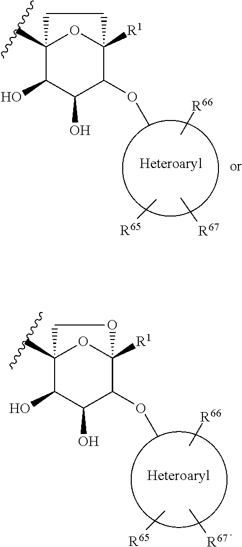

13. The compound of any one of embodiments 1-5, wherein the ASGPR Binding Ligand is

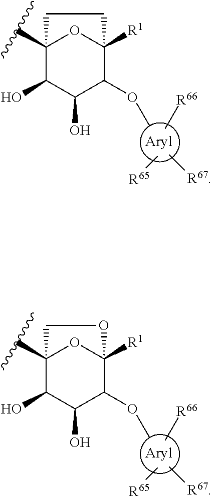

14. The compound of any one of embodiments 1-5, wherein the ASGPR Binding Ligand is

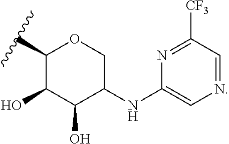 [SMILES: *[C@H]1OCC(Nc2cncc(C(F)(F)F)n2)[C@@H](O)[C@H]1O]

15. The compound of any one of embodiments 1-5, wherein the ASGPR Binding Ligand is

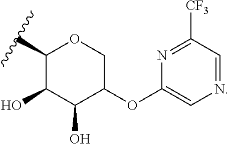 [SMILES: *[C@H]1OCC(Oc2cncc(C(F)(F)F)n2)[C@@H](O)[C@H]1O]

16. The compound of any one of embodiments 1-5, wherein the ASGPR Binding Ligand is

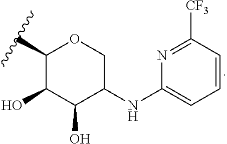 [SMILES: *[C@H]1OCC(Nc2cccc(C(F)(F)F)n2)[C@@H](O)[C@H]1O]

17. The compound of any one of embodiments 1-5, wherein the ASGPR Binding Ligand is

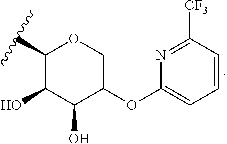 [SMILES: *[C@H]1OCC(Oc2cccc(C(F)(F)F)n2)[C@@H](O)[C@H]1O]

18. The compound of any one of embodiments 1-5, wherein the ASGPR Binding Ligand is

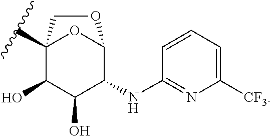 [SMILES: *[C@@]12CO[C@@H](O1)[C@H](Nc1cccc(C(F)(F)F)n1)[C@@H](O)[C@H]2O]

19. The compound of any one of embodiments 1-5, wherein the ASGPR Binding Ligand is

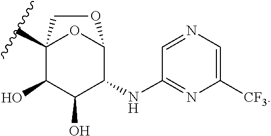 [SMILES: *[C@@]12CO[C@@H](O1)[C@H](Nc1cncc(C(F)(F)F)n1)[C@@H](O)[C@H]2O]

20. The compound of any one of embodiments 1-19, wherein Base is:

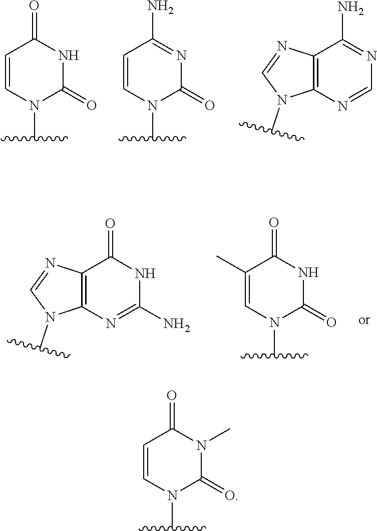

21. The compound of any one of embodiments 1-19, wherein Base is:

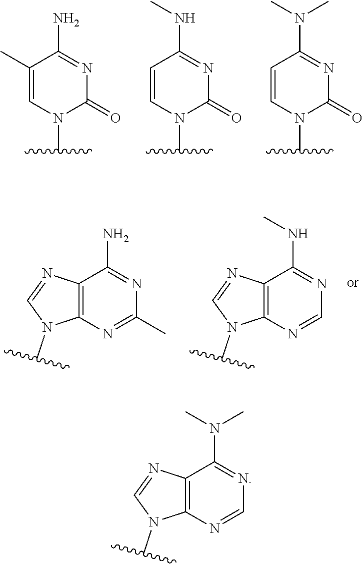

22. The compound of any one of embodiments 1-19, wherein Base is:

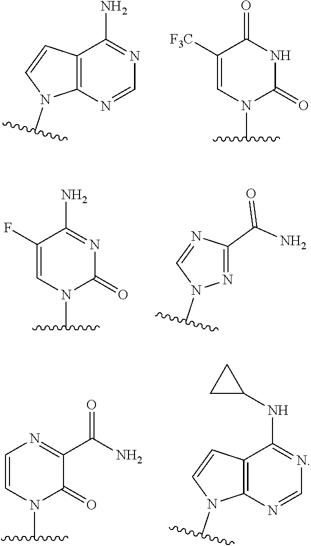 [SMILES: *n1ccc2c(N)ncnc21 ; *n1cc(C(F)(F)F)c(=O)[nH]c1=O ; *n1cc(F)c(N)nc1=O ; *n1cnc(C(N)=O)n1 ; *n1ccnc(C(N)=O)c1=O ; *n1ccc2c(NC3CC3)ncnc21]

23. The compound of any one of embodiments 1-19, wherein Base is:

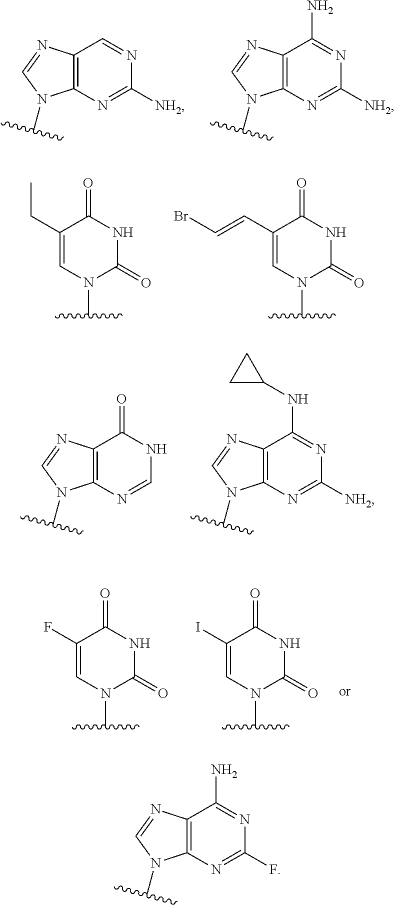

24. The compound of any one of embodiments 1-19, wherein Cleavable Moiety is bond.
25. The compound of any one of embodiments 1-19, wherein Cleavable Moiety is

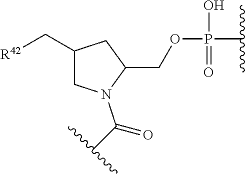

wherein the Therapeutic Oligonucleotide is attached to the —P—.
26. The compound of any one of embodiments 1-19, wherein Cleavable Moiety is

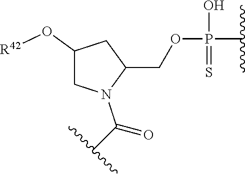

wherein the Therapeutic Oligonucleotide is attached to the —P—.
27. The compound of any one of embodiments 1-23, wherein Cleavable Moiety is

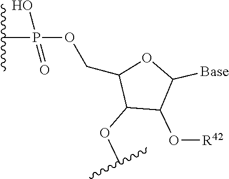

wherein the Therapeutic Oligonucleotide is attached to the —P—.
28. The compound of any one of embodiments 1-23, wherein Cleavable Moiety is

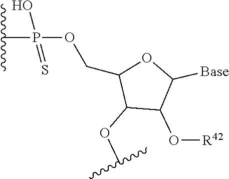

wherein the Therapeutic Oligonucleotide is attached to the —P—.
29. The compound of any one of embodiments 1-23, wherein Cleavable Moiety is

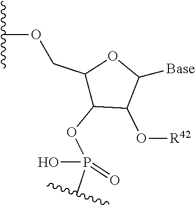

wherein the Therapeutic Oligonucleotide is attached to the —P—.
30. The compound of any one of embodiments 1-23, wherein Cleavable Moiety is

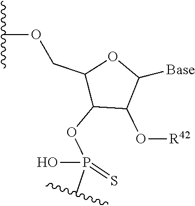

wherein the Therapeutic Oligonucleotide is attached to the —P—.
31. The compound of any one of embodiments 1-23, wherein Cleavable Moiety is

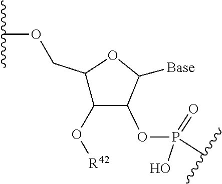

wherein the Therapeutic Oligonucleotide is attached to the —P—.
32. The compound of any one of embodiments 1-23, wherein Cleavable Moiety is

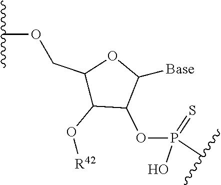

wherein the Therapeutic Oligonucleotide is attached to the —P—.
33. The compound of any one of embodiments 1-23, wherein Cleavable Moiety is

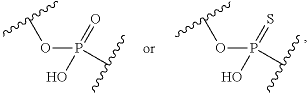 [SMILES: *OP(*)(=O)O ; *OP(*)(O)=S]

wherein the Therapeutic Oligonucleotide is attached to the —P—.
34. The compound of any one of embodiments 1-23, wherein Cleavable Moiety is selected from the group consisting of

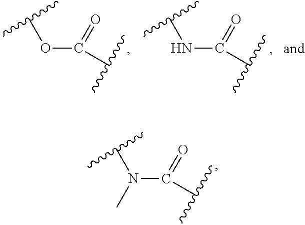

wherein the Therapeutic Oligonucleotide is attached to the —C—.
35. The compound of any one of embodiments 27-34, wherein R⁴² is methyl.
36. The compound of embodiment 1, wherein the compound is selected from the group consisting of:

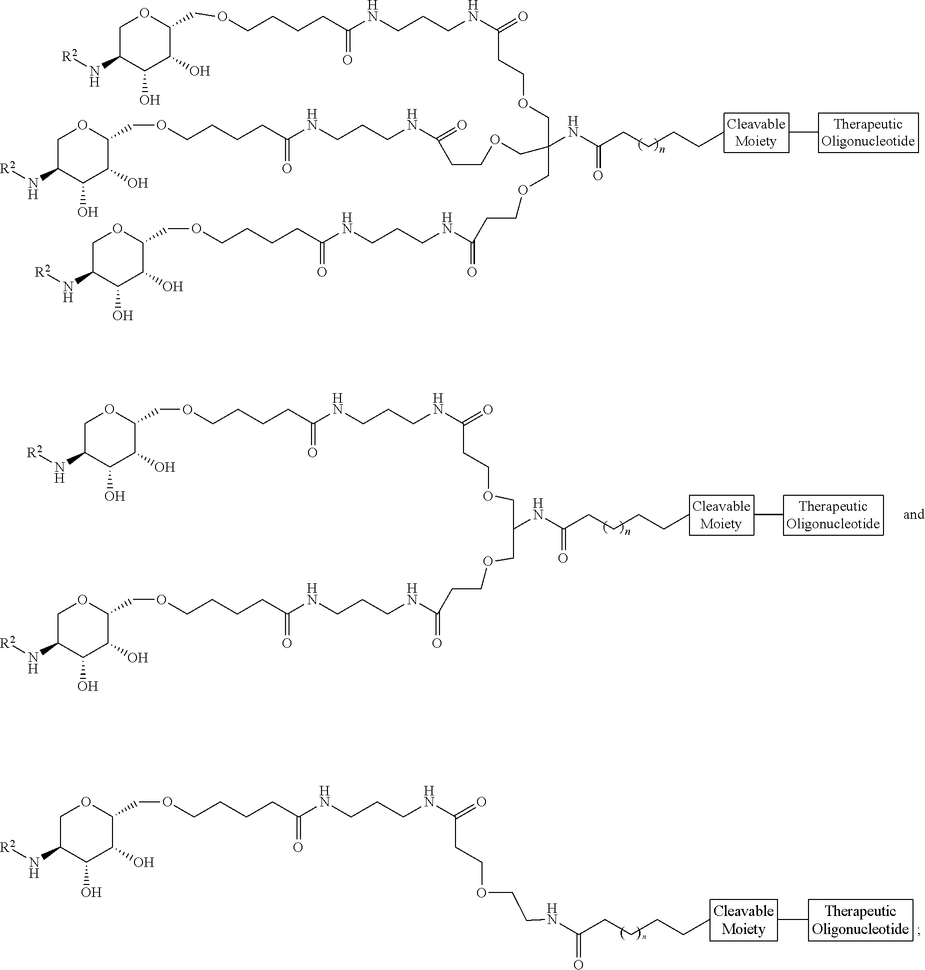

or a pharmaceutically acceptable salt thereof,
   wherein
   n is 0, 1, 2, 3, 4, 5, 6, 7, 8, 9, or 10; and
   R² is

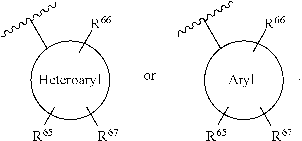

37. The compound of embodiment 36 wherein the compound is

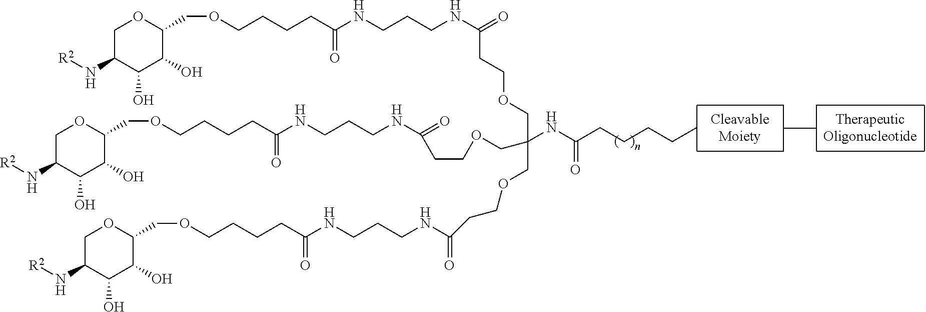

or a pharmaceutically acceptable salt thereof.
38. The compound of embodiment 36 wherein the compound is

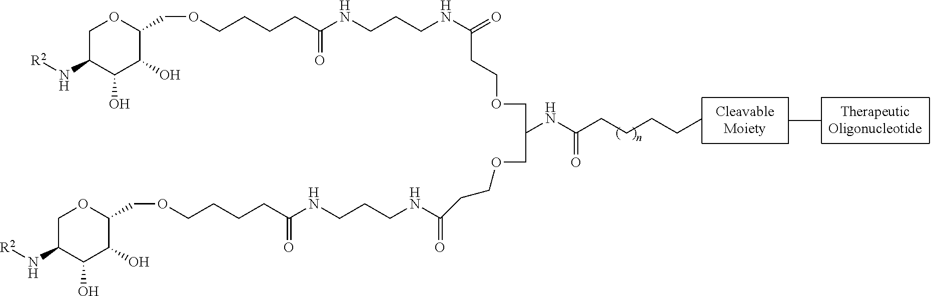

or a pharmaceutically acceptable salt thereof.
39. The compound of embodiment 36 wherein the compound is

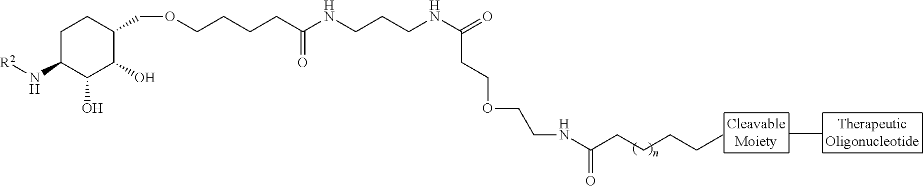

or a pharmaceutically acceptable salt thereof.
40. The compound of embodiment 1, wherein the compound is selected from the group consisting of:

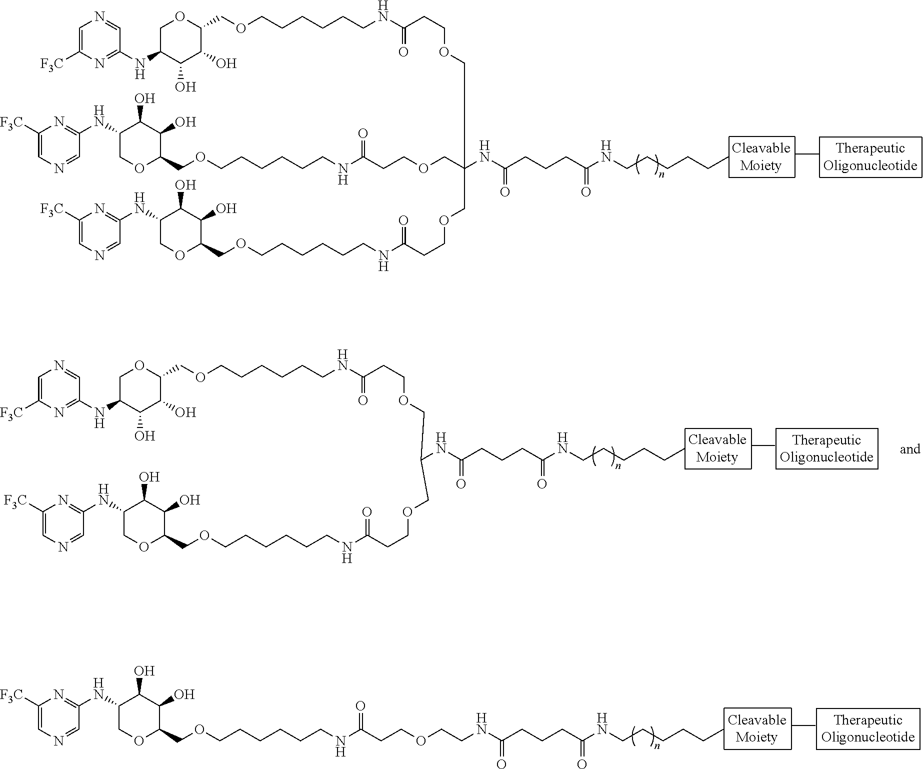

or a pharmaceutically acceptable salt thereof;
   wherein
   n is 0, 1, 2, 3, 4, 5, 6, 7, 8, 9, or 10.
41. The compound of any embodiment 40 wherein the compound is of Formula

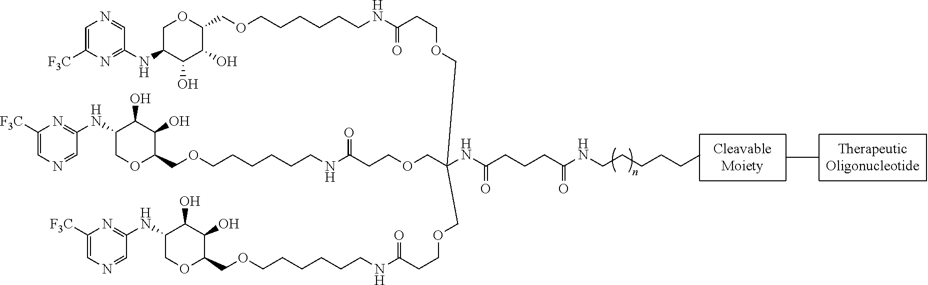

or a pharmaceutically acceptable salt thereof.
42. The compound ofany embodiment 40 wherein the compound is of Formula

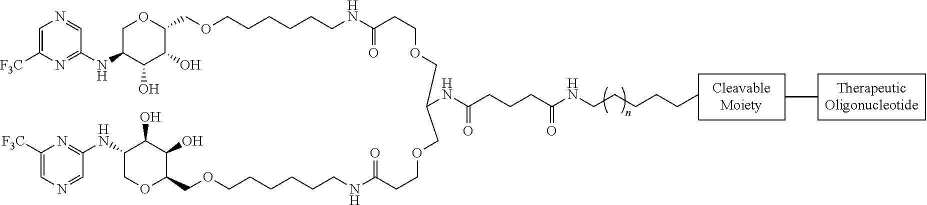

or a pharmaceutically acceptable salt thereof
43. The compound of any embodiment 40 wherein the compound is of Formula

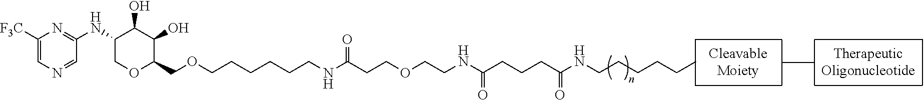

or a pharmaceutically acceptable salt thereof.
44. The compound of embodiment 1, wherein the compound is selected from the group consisting of:

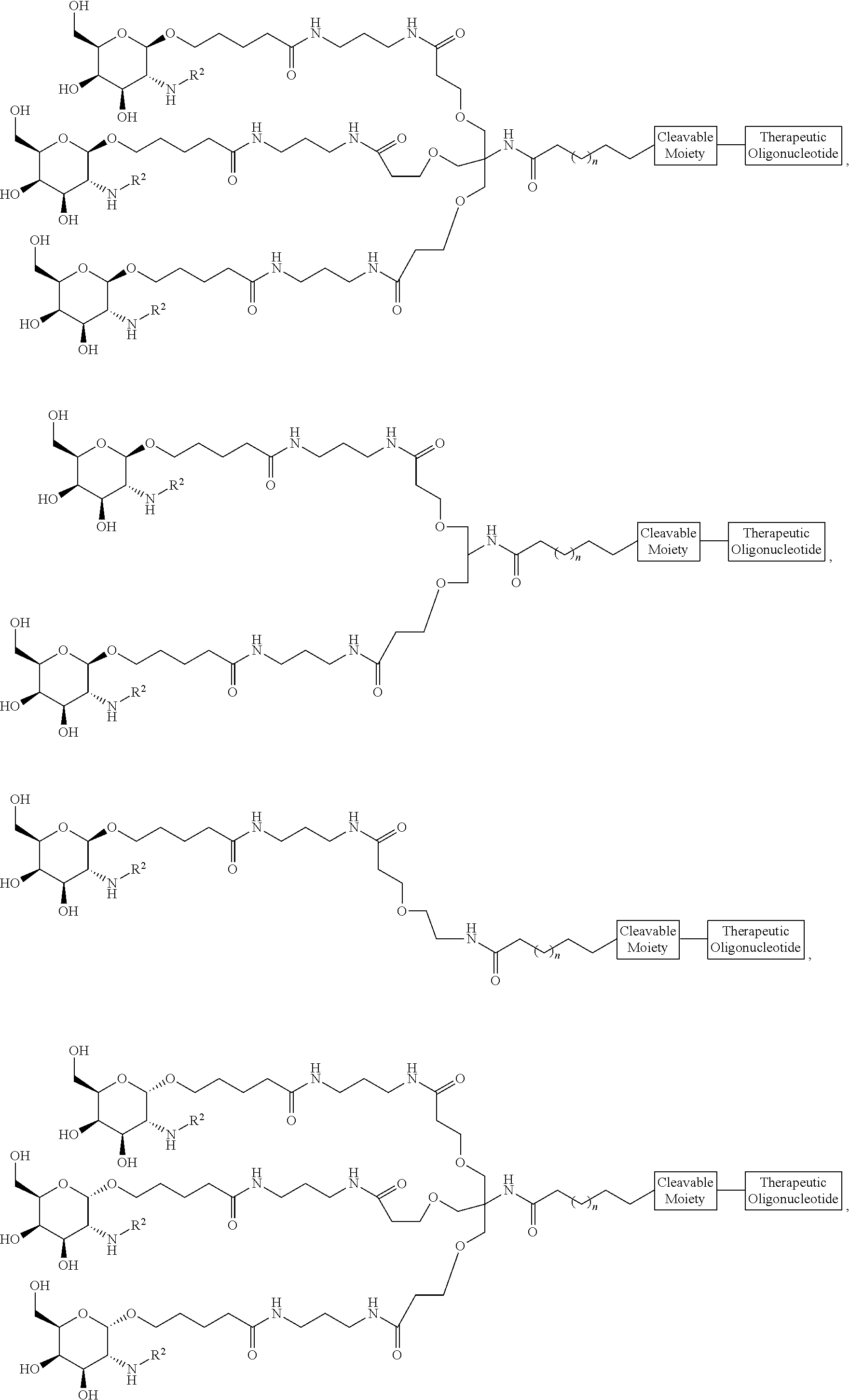

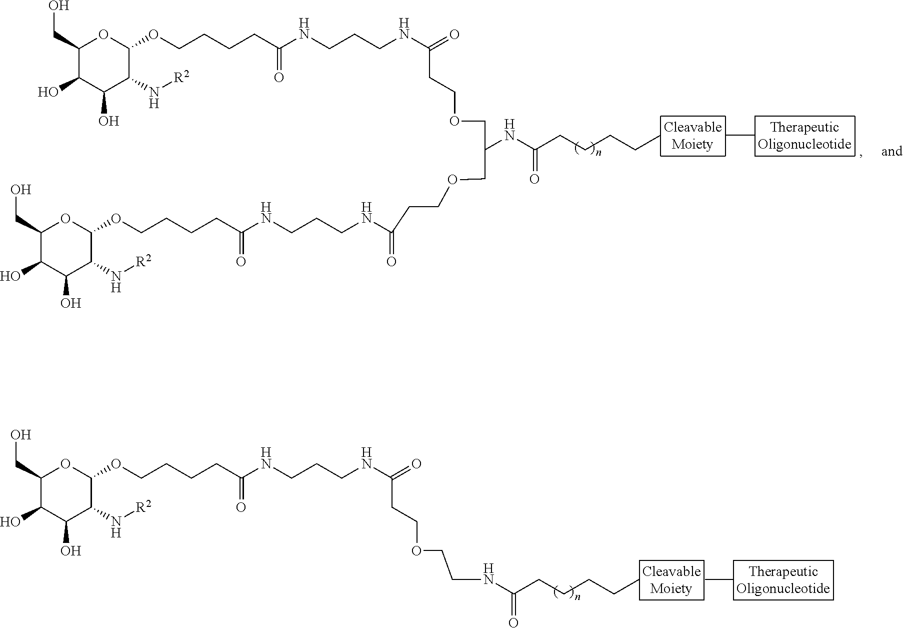

or a pharmaceutically acceptable salt thereof,
   wherein
   n is 0, 1, 2, 3, 4, 5, 6, 7, 8, 9, or 10; and
   R² is

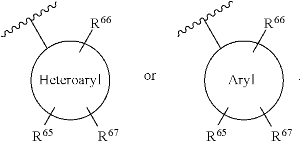

45. The compound of embodiment 1, wherein the compound is selected from the group consisting of:

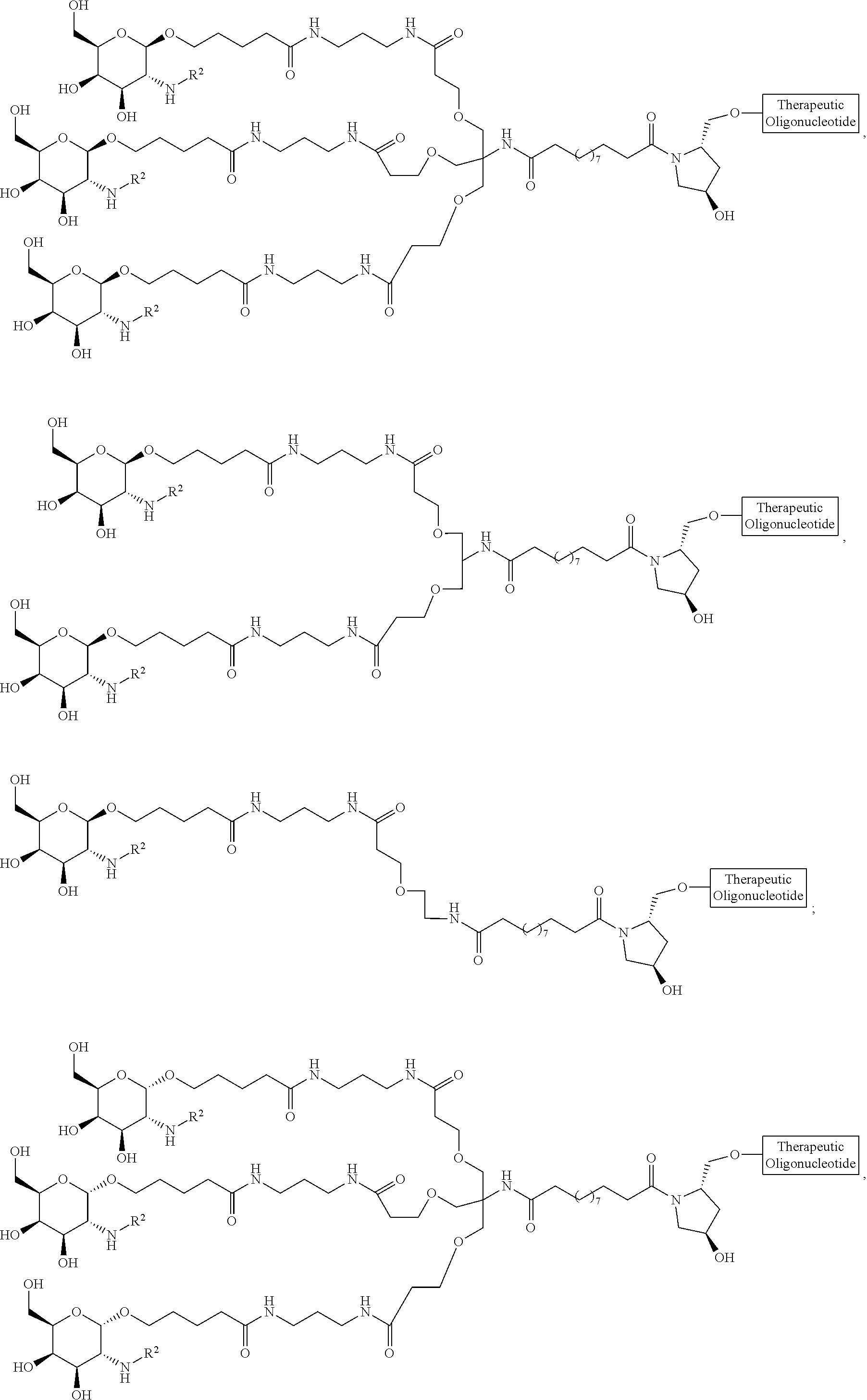

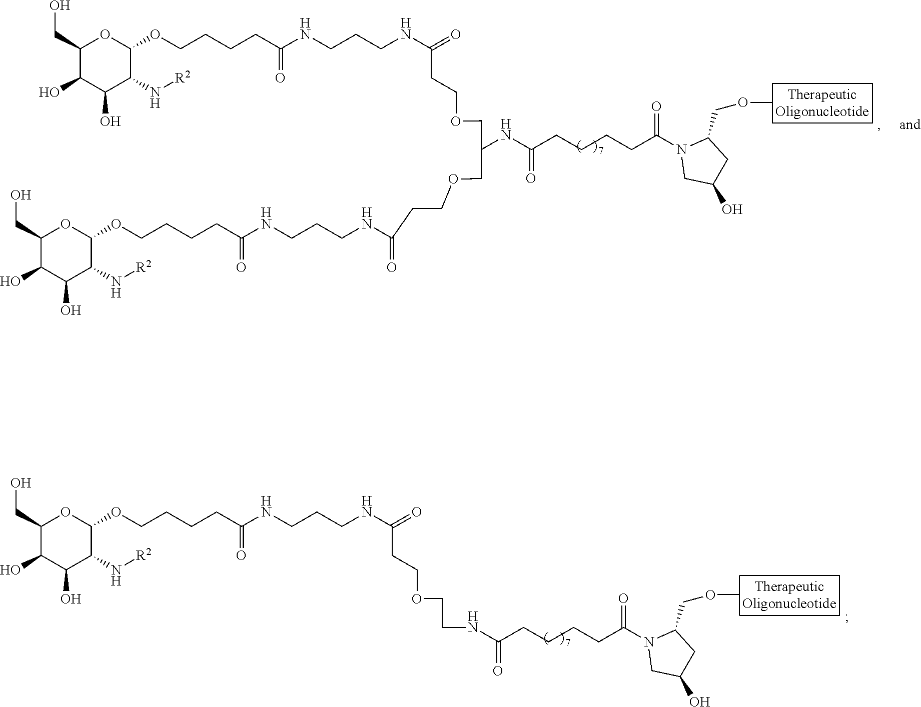

or a pharmaceutically acceptable salt thereof,
   wherein
   n is 0, 1, 2, 3, 4, 5, 6, 7, 8, 9, or 10; and
   R² is

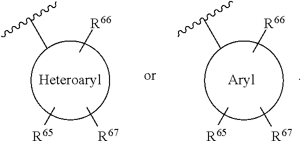

46. The compound of any one of embodiments 1-45, wherein Therapeutic Oligonucleotide is a nucleotide described in Table 1.
47. The compound of any one of embodiments 1-45, wherein Therapeutic Oligonucleotide is a nucleotide that is at least 80% identical to a nucleotide described in Table 1.
48. The compound of any one of embodiments 1-45, wherein Therapeutic Oligonucleotide is a nucleotide that is at least 85% identical to a nucleotide described in Table 1.
49. The compound of any one of embodiments 1-45, wherein Therapeutic Oligonucleotide is a nucleotide that is at least 90% identical to a nucleotide described in Table 1.
50. The compound of any one of embodiments 1-45, wherein Therapeutic Oligonucleotide is a nucleotide that is at least 95% identical to a nucleotide described in Table 1.
51. The compound of any one of embodiments 1-45, wherein Therapeutic Oligonucleotide is a nucleotide that is at least 96% identical to a nucleotide described in Table 1.
52. The compound of any one of embodiments 1-45, wherein Therapeutic Oligonucleotide is a nucleotide that is at least 97% identical to a nucleotide described in Table 1.
53. The compound of any one of embodiments 1-45, wherein Therapeutic Oligonucleotide is a nucleotide that is at least 98% identical to a nucleotide described in Table 1.
54. The compound of any one of embodiments 1-45, wherein Therapeutic Oligonucleotide is a nucleotide that is at least 99% identical to a nucleotide described in Table 1.
55. The compound of any one of embodiments 1-54, wherein the Therapeutic Oligonucleotide is Therapeutic Oligonucleotide 1.
56. The compound of any one of embodiments 1-54, wherein the Therapeutic Oligonucleotide is Therapeutic Oligonucleotide 2.
57. The compound of any one of embodiments 1-54, wherein the Therapeutic Oligonucleotide is Therapeutic Oligonucleotide 3.
58. The compound of any one of embodiments 1-54, wherein the Therapeutic Oligonucleotide is Therapeutic Oligonucleotide 4.
59. The compound of any one of embodiments 1-54, wherein the Therapeutic Oligonucleotide is Therapeutic Oligonucleotide 5.
60. The compound of any one of embodiments 1-54, wherein the Therapeutic Oligonucleotide is Therapeutic Oligonucleotide 6.
61. The compound of any one of embodiments 1-54, wherein the Therapeutic Oligonucleotide is Therapeutic Oligonucleotide 7.
62. The compound of any one of embodiments 1-54, wherein the Therapeutic Oligonucleotide is Therapeutic Oligonucleotide 8.
63. The compound of any one of embodiments 1-54, wherein the Therapeutic Oligonucleotide is Therapeutic Oligonucleotide 9.
64. The compound of any one of embodiments 1-54, wherein the Therapeutic Oligonucleotide is Therapeutic Oligonucleotide 10.
65. The compound of any one of embodiments 1-54, wherein the Therapeutic Oligonucleotide is Therapeutic Oligonucleotide 11.
66. The compound of any one of embodiments 1-54, wherein the Therapeutic Oligonucleotide is Therapeutic Oligonucleotide 12.
67. The compound of any one of embodiments 1-54, wherein the Therapeutic Oligonucleotide is Therapeutic Oligonucleotide 13.
68. The compound of any one of embodiments 1-54, wherein the Therapeutic Oligonucleotide is Therapeutic Oligonucleotide 14.
69. The compound of any one of embodiments 1-54, wherein the Therapeutic Oligonucleotide is Therapeutic Oligonucleotide 15.
70. The compound of any one of embodiments 1-54, wherein the Therapeutic Oligonucleotide is Therapeutic Oligonucleotide 16.
71. The compound of any one of embodiments 1-54, wherein the Therapeutic Oligonucleotide is Therapeutic Oligonucleotide 17.
72. The compound of any one of embodiments 1-54, wherein the Therapeutic Oligonucleotide is Therapeutic Oligonucleotide 18.
73. The compound of any one of embodiments 1-54, wherein the Therapeutic Oligonucleotide is Therapeutic Oligonucleotide 19.
74. The compound of any one of embodiments 1-54, wherein the Therapeutic Oligonucleotide is Therapeutic Oligonucleotide 20.
75. The compound of any one of embodiments 1-54, wherein the Therapeutic Oligonucleotide is Therapeutic Oligonucleotide 21.
76. The compound of any one of embodiments 1-54, wherein the Therapeutic Oligonucleotide is Therapeutic Oligonucleotide 22.
77. The compound of any one of embodiments 1-54, wherein the Therapeutic Oligonucleotide is Therapeutic Oligonucleotide 23.
78. The compound of any one of embodiments 1-54, wherein the Therapeutic Oligonucleotide is Therapeutic Oligonucleotide 24.
79. The compound of any one of embodiments 1-54, wherein the Therapeutic Oligonucleotide is Therapeutic Oligonucleotide 25.
80. The compound of any one of embodiments 1-54, wherein the Therapeutic Oligonucleotide is Therapeutic Oligonucleotide 26.
81. The compound of any one of embodiments 1-54, wherein the Therapeutic Oligonucleotide is Therapeutic Oligonucleotide 27.
82. The compound of any one of embodiments 1-54, wherein the Therapeutic Oligonucleotide is Therapeutic Oligonucleotide 28.
83. The compound of any one of embodiments 1-54, wherein the Therapeutic Oligonucleotide is Therapeutic Oligonucleotide 29.
84. A compound selected from

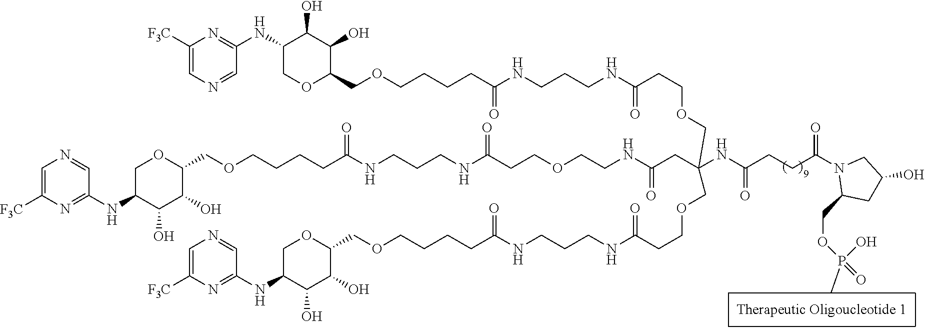

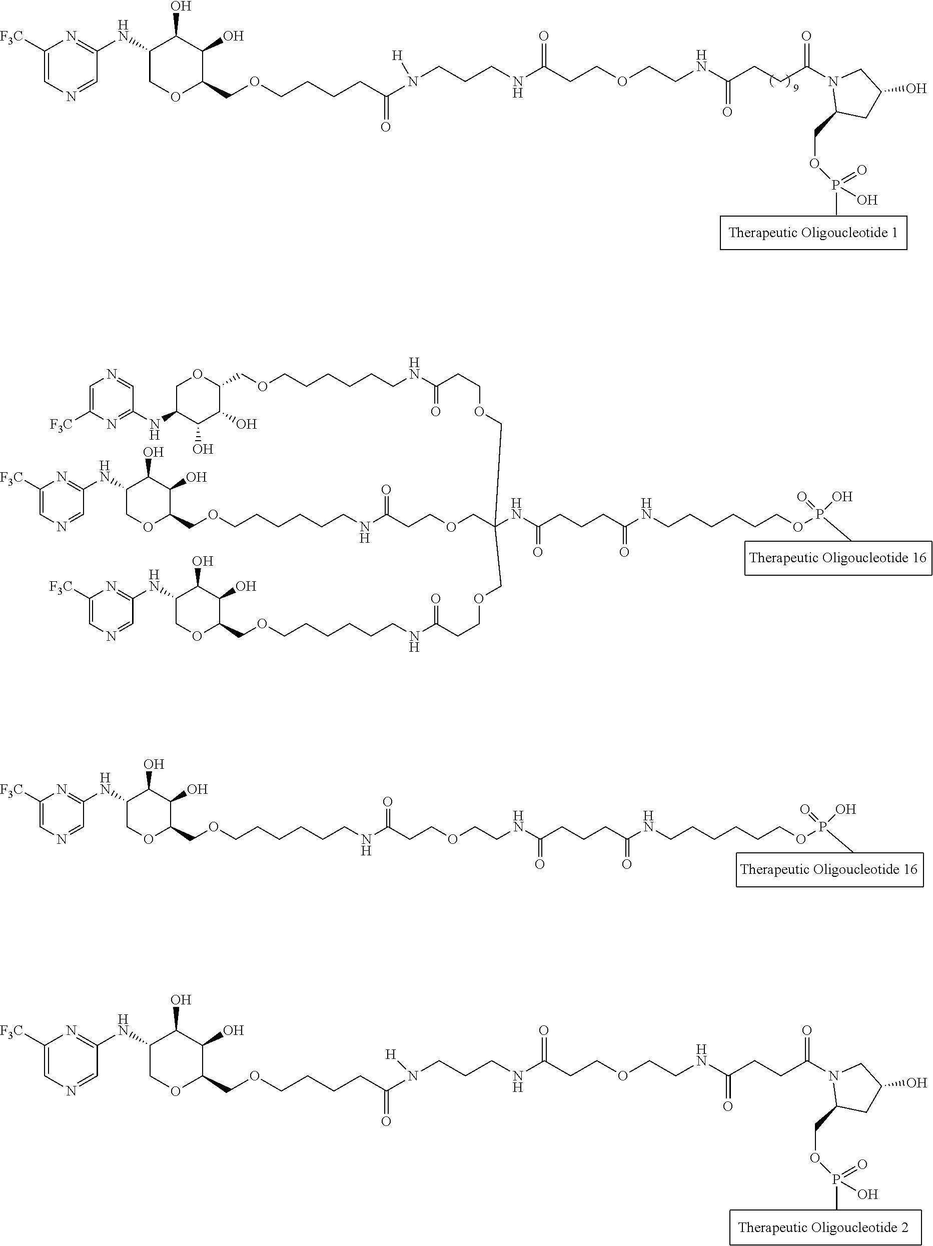

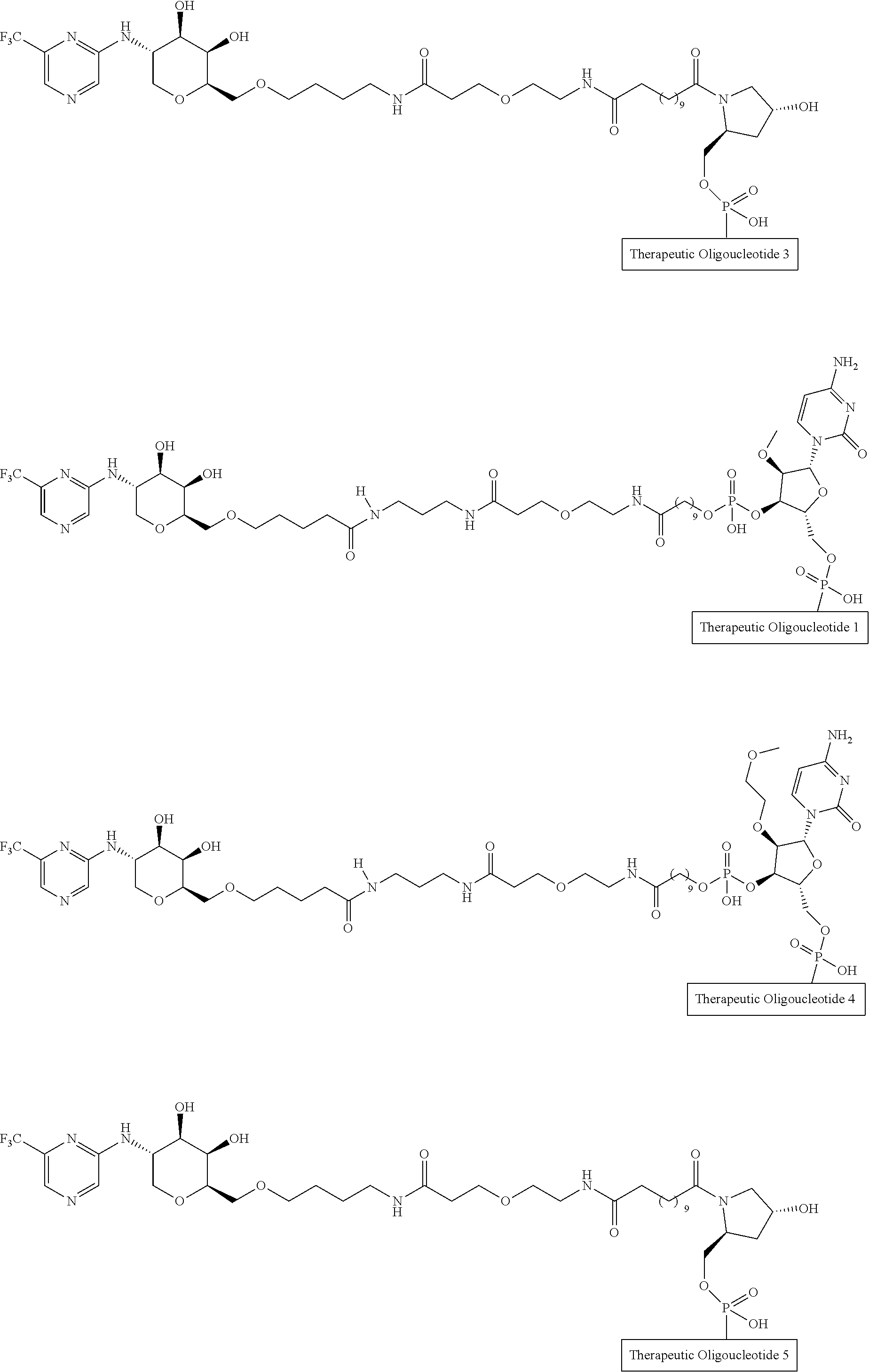

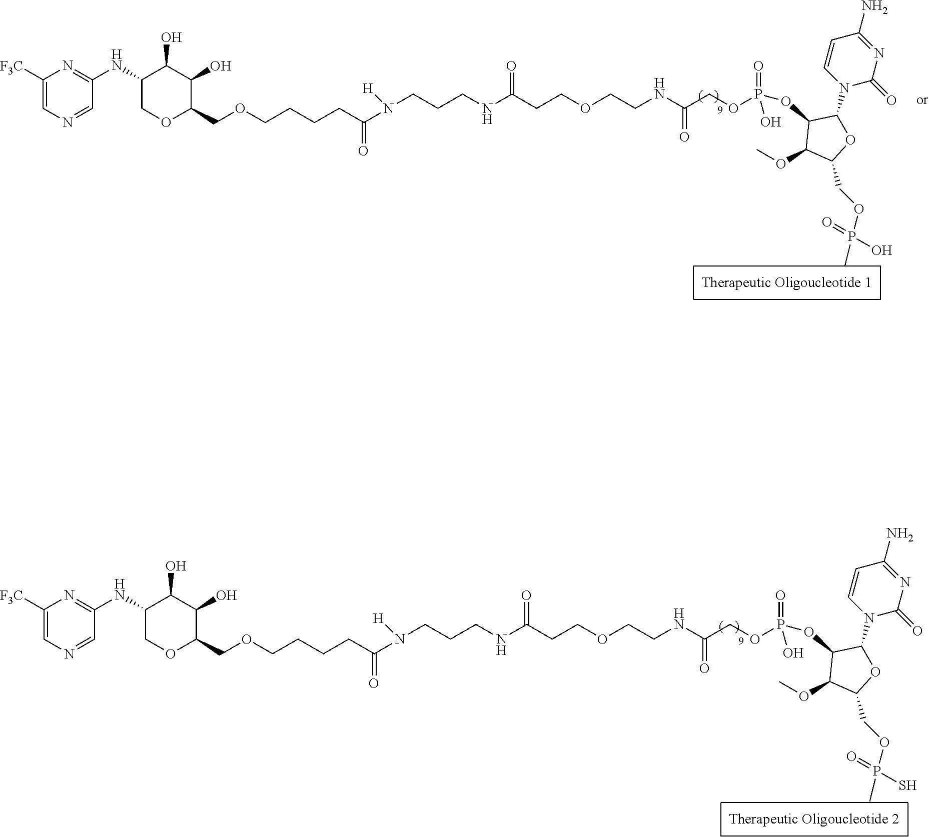

or a pharmaceutically acceptable salt thereof.
85. A pharmaceutical composition comprising a compound of any one of embodiments 1-84 or a pharmaceutically acceptable salt thereof and a pharmaceutically acceptable excipient.
86. A method for the treatment of a disorder comprising administering an effective amount of a compound of any one of embodiments 1-84 or a pharmaceutically acceptable salt thereof or a pharmaceutical composition thereof to a human patient.
87. The method of embodiment 86, wherein the disorder responds to treatment with the Therapeutic Oligonucleotide.
88. The method of embodiment 86 or 87, wherein the disorder is a liver associated disorder.
89. The method of embodiment 86 or 87, wherein the disorder is selected from acute hepatic porphyria (AHP), hyperoxaluria type 1 (PH1), hypercholesterolemia, hemophilia, a complement mediated disorder, HBV, hypertension, nonalcoholic steatohepatitis (NASH), a cardiovascular disease, hyperoxaluria type 2, hyperoxaluria type 3, β-thalassemia, immunoglobulin A nephropathy (IgAN), angioedema, myelodysplastic disorder, and treatment resistant hypertension.

In certain embodiments, the invention includes:
1. A compound of Formula:

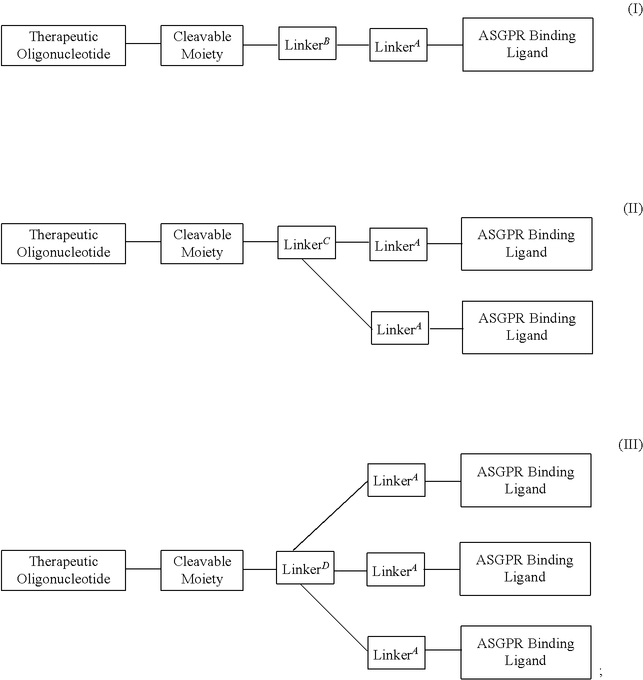

or a pharmaceutically acceptable salt thereof,
wherein:
ASGPR Binding Ligand is a compound selected from:

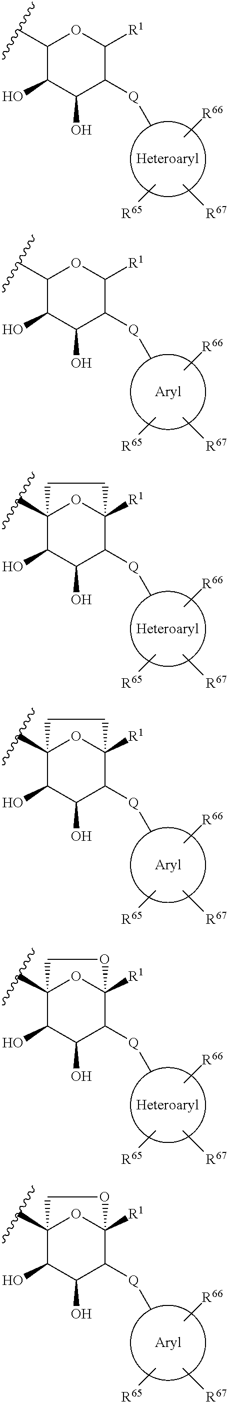

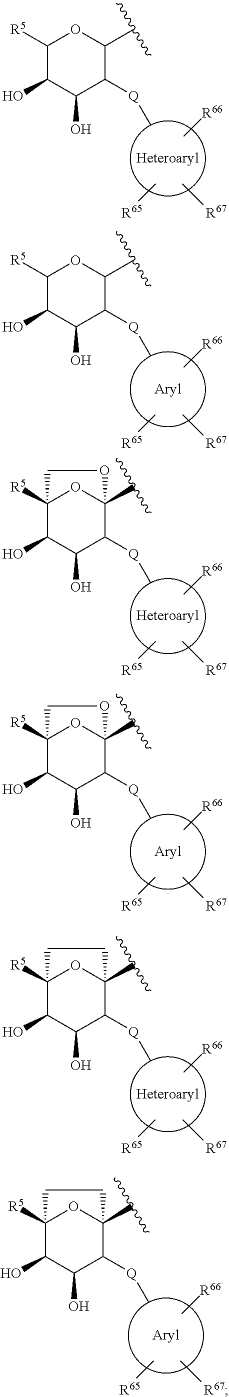

Q is —O— or —N(R¹⁰)—;
R¹ and R⁵ are independently selected from hydrogen, alkyl, alkenyl, alkynyl, haloalkyl, halogen, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, C₀-C₆alkyl-N═S(O)(R³)₂, C₀-C₆alkylN₃, and C₀-C₆alkyl-O—S(O)₂R³;
R³ at each occurrence is independently selected from the group consisting of hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from the group consisting of hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is hydrogen, alkyl, haloalkyl, arylalkyl, heteroarylalkyl, alkenyl, alkynyl, aryl, heteroaryl, heterocycle, C(O)R³, S(O)R³, C(S)R³, or S(O)₂R³;
R⁶⁵, R⁶⁶, and R⁶⁷ are independently selected at each occurrence from the group consisting of hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R′)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰;
R⁸⁰ is independently selected at each instance from the group consisting of alkyl, alkenyl, alkynyl, haloalkyl, —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.
Cleavable Moiety is a bond,

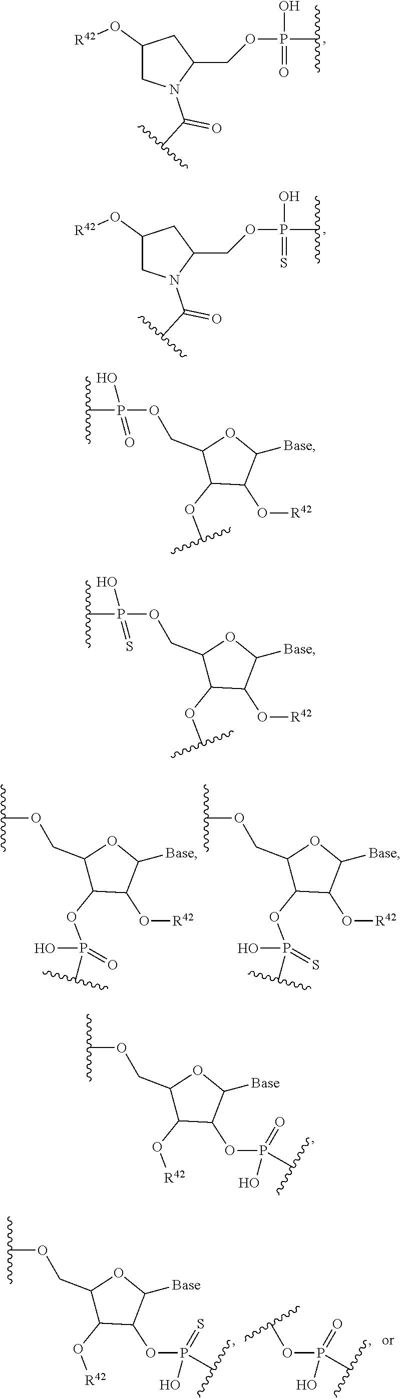

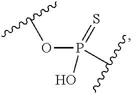 [SMILES: *OP(*)(O)=S]

wherein the Therapeutic Oligonucleotide is attached to the —P—, or
Cleavable Moiety is

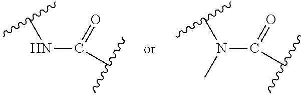

or wherein the Therapeutic Oligonucleotide is bound to the —C— or —N—, or
Cleavable Moiety is

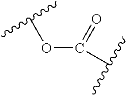 [SMILES: *OC(*)=O]

wherein the Therapeutic Oligonucleotide is bound to the —C— or —O—;
R⁴² is hydrogen, alkyl, alkyl-O-alkyl, or C(O)R⁴³.

R⁴³ is selected from the group consisting of hydrogen, alkyl, and haloalkyl;
Base is a heterocycle comprising two, three, or four nitrogen atoms, wherein the heterocycle is substituted with an oxo, amino, or carbamoyl and optionally substituted with 1, 2, or 3 substituents independently selected from oxo, alkyl, haloalkyl, alkenyl, haloalkenyl, and halogen;
Therapeutic Oligonucleotide is an antisense oligonucleotide, siRNA, shRNA, DNA aptamer, RNA aptamer, miRNA, miRNA mimic, antimiRNA, DNA decoy, RNA decoy, or CpG oligonucleotide;
Linker^{A} and Linker^{B} are independently selected from:

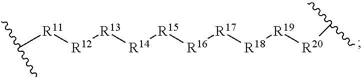

Linker^{C} is selected from:

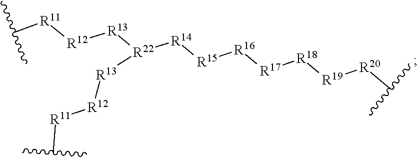

Linker^{D} is selected from:

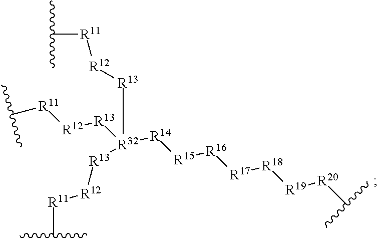

R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[C(O)—CH₂—NR⁶]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, and 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R²² is independently at each occurrence selected from the group consisting of alkyl, —C(O)N—, —NC(O)—, —N—, —C(R²¹)—, —P(0)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹; and
X⁻ is an anionic group.
2. The compound of embodiment D1, wherein Cleavable Moiety is a bond

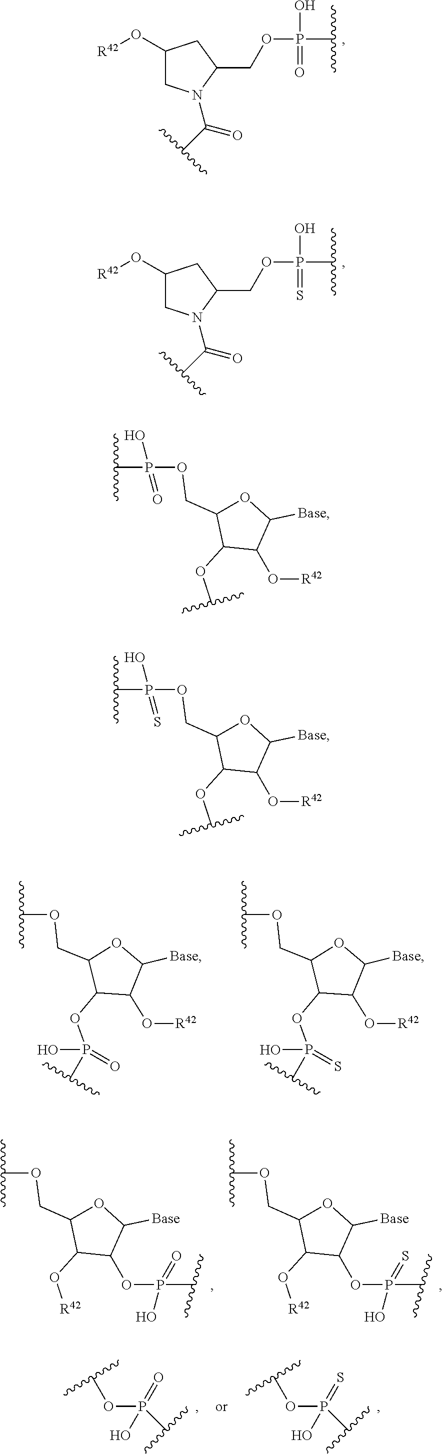

wherein the Therapeutic Oligonucleotide is attached to the —P—;
or

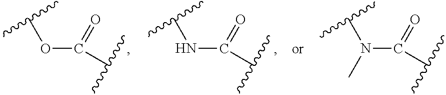

wherein the Therapeutic Oligonucleotide is bound to the —C—;
Base is a heterocycle comprising two, three, or four nitrogen atoms, wherein the heterocycle is substituted with an oxo, amino, or carbamoyl and optionally substituted with 1, 2, or 3 substituents independently selected from alkyl, haloalkyl, and halogen; and
R¹ and R⁵ are independently selected from hydrogen, alkyl, alkenyl, alkynyl, haloalkyl, halogen, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³.
3. The compound of embodiment D1, wherein R¹ is hydrogen.
4. The compound of any one of embodiments D1-D3, wherein the compound is of formula:

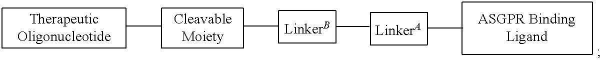

or a pharmaceutically acceptable salt thereof.
5. The compound of any one of embodiments D1-D3, wherein the compound is of formula:

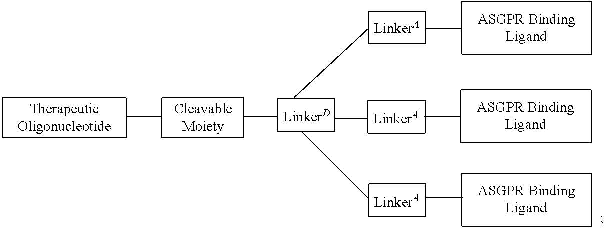

or a pharmaceutically acceptable salt thereof.
6. The compound of any one of embodiments D1-D3, wherein the compound is of formula:

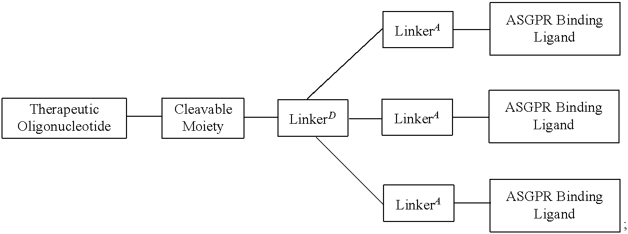

or a pharmaceutically acceptable salt thereof.
7. The compound of any one of embodiments D1-D6, wherein the ASGPR Binding Ligand is

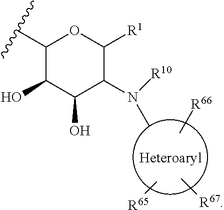

8. The compound of any one of embodiments D1-D6, wherein the ASGPR Binding Ligand is

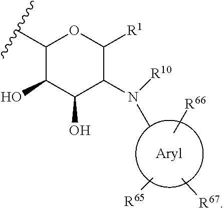

9. The compound of any one of embodiments D1-D6, wherein the ASGPR Binding Ligand is

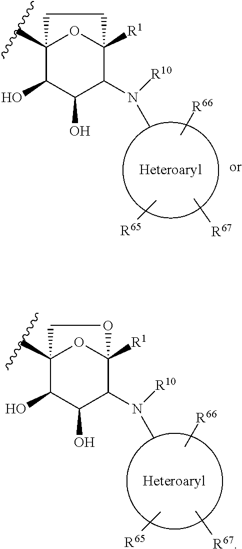

10. The compound of any one of embodiments D1-D6, wherein the ASGPR Binding Ligand is

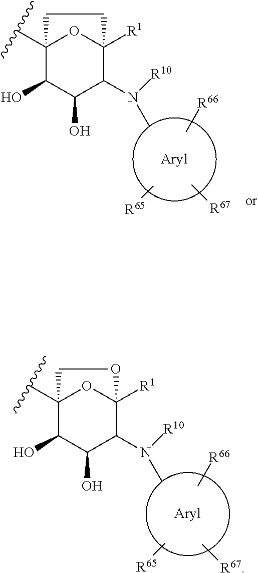

11. The compound of any one of embodiments D1-D6, wherein the ASGPR Binding Ligand is

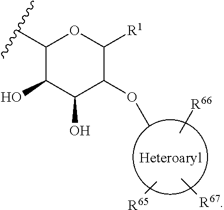

12. The compound of any one of embodiments D1-D6, wherein the ASGPR Binding Ligand is

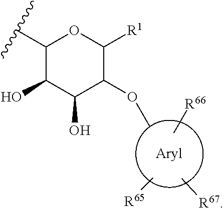

13. The compound of any one of embodiments D1-D6, wherein the ASGPR Binding Ligand is

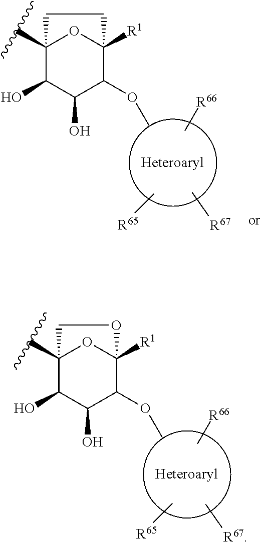

14. The compound of any one of embodiments D1-D6, wherein the ASGPR Binding Ligand is

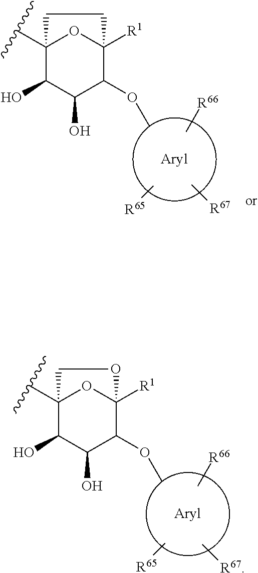

15. The compound of any one of embodiments D1-D6, wherein the ASGPR Binding Ligand is

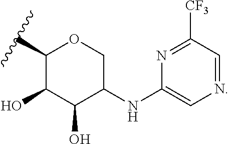 [SMILES: *[C@H]1OCC(Nc2cncc(C(F)(F)F)n2)[C@@H](O)[C@H]1O]

16. The compound of any one of embodiments D1-D6, wherein the ASGPR Binding Ligand is

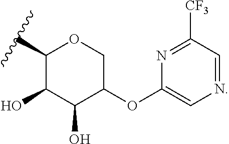 [SMILES: *[C@H]1OCC(Oc2cncc(C(F)(F)F)n2)[C@@H](O)[C@H]1O]

17. The compound of any one of embodiments D1-D6, wherein the ASGPR Binding Ligand is

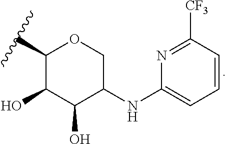 [SMILES: *[C@H]1OCC(Nc2cccc(C(F)(F)F)n2)[C@@H](O)[C@H]1O]

18. The compound of any one of embodiments D1-D6, wherein the ASGPR Binding Ligand is

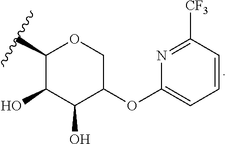 [SMILES: *[C@H]1OCC(Oc2cccc(C(F)(F)F)n2)[C@@H](O)[C@H]1O]

19. The compound of any one of embodiments D1-D6, wherein the ASGPR Binding Ligand is

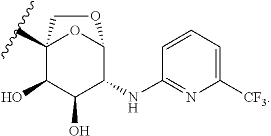 [SMILES: *[C@@]12CO[C@@H](O1)[C@H](Nc1cccc(C(F)(F)F)n1)[C@@H](O)[C@H]2O]

20. The compound of any one of embodiments D1-D6, wherein the ASGPR Binding Ligand is

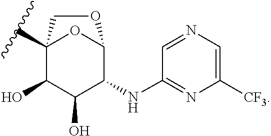 [SMILES: *[C@@]12CO[C@@H](O1)[C@H](Nc1cncc(C(F)(F)F)n1)[C@@H](O)[C@H]2O]

21. The compound of any one of embodiments D1-D20, wherein Base is:

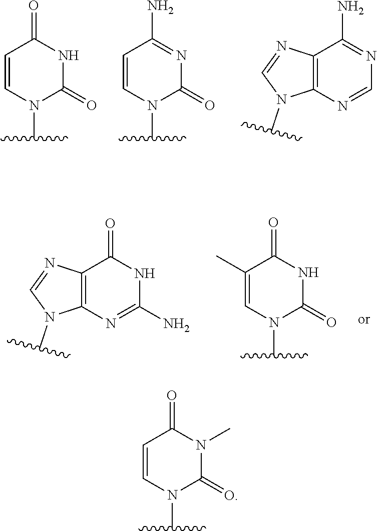

22. The compound of any one of embodiments D1-D20, wherein Base is:

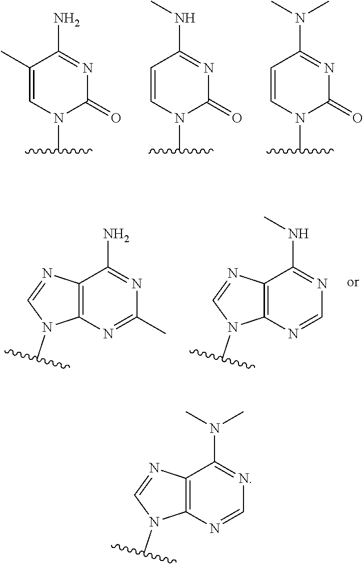

23. The compound of any one of embodiments D1-D20, wherein Base is:

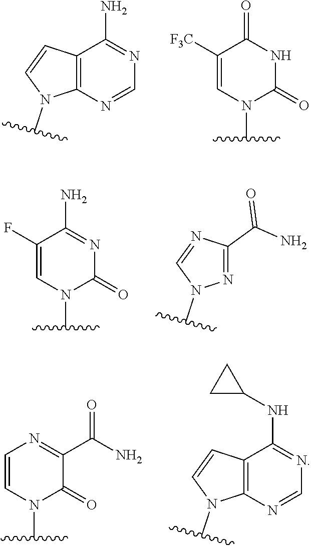 [SMILES: *n1ccc2c(N)ncnc21 ; *n1cc(C(F)(F)F)c(=O)[nH]c1=O ; *n1cc(F)c(N)nc1=O ; *n1cnc(C(N)=O)n1 ; *n1ccnc(C(N)=O)c1=O ; *n1ccc2c(NC3CC3)ncnc21]

24. The compound of any one of embodiments D1-D20, wherein Base is:

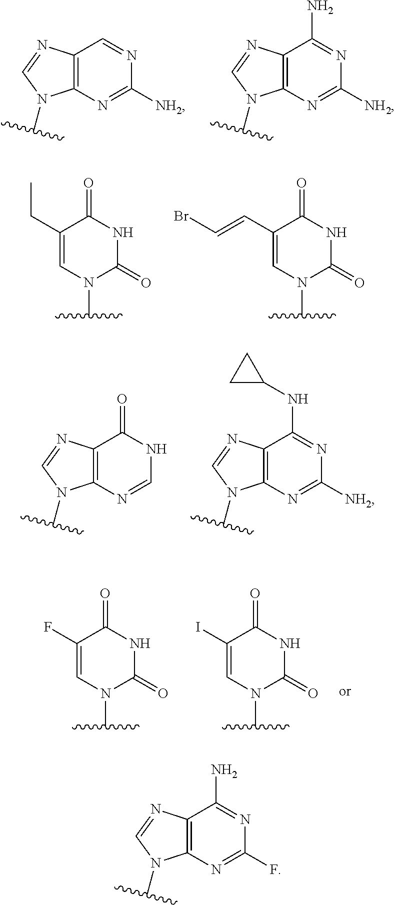

25. The compound of any one of embodiments D1-D20, wherein Cleavable Moiety is bond.
26. The compound of any one of embodiments D1-D20, wherein Cleavable Moiety is

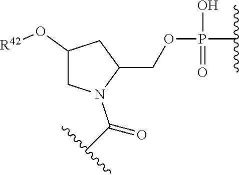

wherein the Therapeutic Oligonucleotide is attached to the —P—.
27. The compound of any one of embodiments D1-D20, wherein Cleavable Moiety is

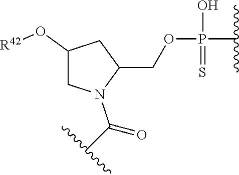

wherein the Therapeutic Oligonucleotide is attached to the —P—.
28. The compound of any one of embodiments D1-D24, wherein Cleavable Moiety is

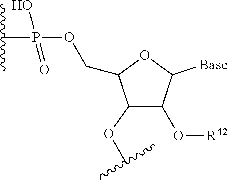

wherein the Therapeutic Oligonucleotide is attached to the —P—.
29. The compound of any one of embodiments D1-D24, wherein Cleavable Moiety is

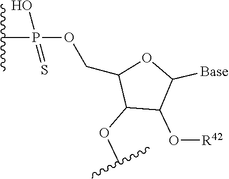

wherein the Therapeutic Oligonucleotide is attached to the —P—.
30. The compound of any one of embodiments D1-D24, wherein Cleavable Moiety is

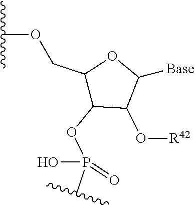

wherein the Therapeutic Oligonucleotide is attached to the —P—.
31. The compound of any one of embodiments D1-D24, wherein Cleavable Moiety is

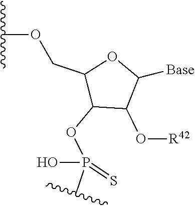

wherein the Therapeutic Oligonucleotide is attached to the —P—.
32. The compound of any one of embodiments D1-D24, wherein Cleavable Moiety is

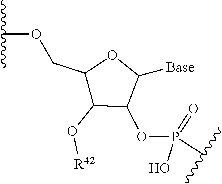

wherein the Therapeutic Oligonucleotide is attached to the —P—.
33. The compound of any one of embodiments D1-D24, wherein Cleavable Moiety is

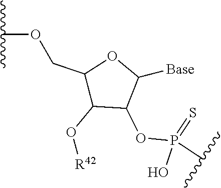

wherein the Therapeutic Oligonucleotide is attached to the —P—.
34. The compound of any one of embodiments D1-D24, wherein Cleavable Moiety is

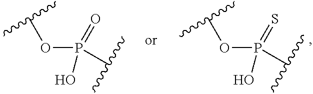 [SMILES: *OP(*)(=O)O ; *OP(*)(O)=S]

wherein the Therapeutic Oligonucleotide is attached to the —P—.
35. The compound of any one of embodiments D1-D24, wherein Cleavable Moiety is

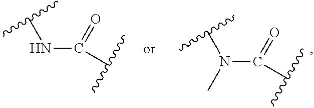

wherein the Therapeutic Oligonucleotide is bound to the —N— or —C—, or

Cleavable Moiety is

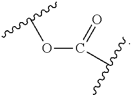 [SMILES: *OC(*)=O]

wherein the Therapeutic Oligonucleotide is bound to the —C— or —O—.
36. The compound of any one of embodiments D26-D33, wherein R⁴² is methyl.
37. The compound of any one of embodiments D1-D36, wherein Linker^{A} is bond.
38. The compound of any one of embodiments D1-D37, wherein

Linker^{B} is

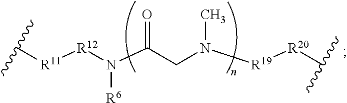

Linker^{C} is

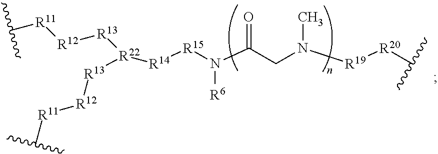

Linker^{D} is

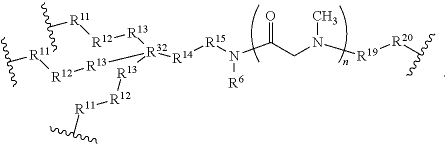

39. The compound of embodiment D1, wherein the compound is selected from the group consisting of:

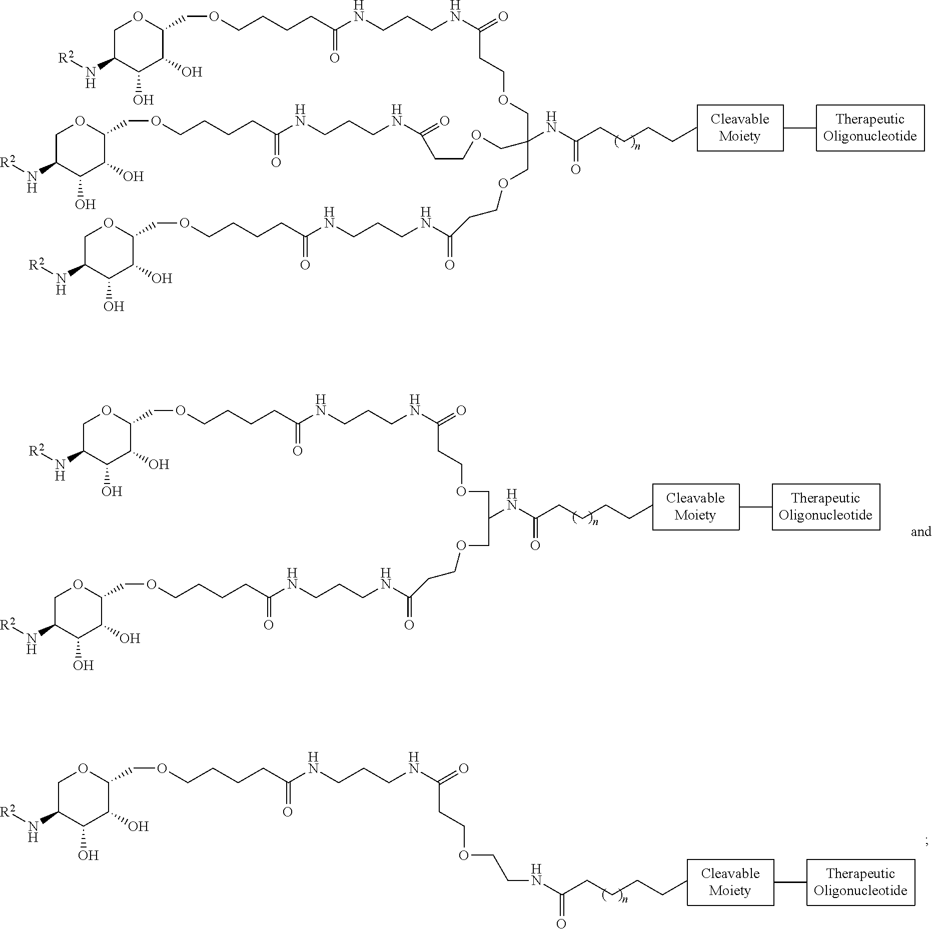

or a pharmaceutically acceptable salt thereof;
   wherein
   n is 0, 1, 2, 3, 4, 5, 6, 7, 8, 9, or 10; and
   R² is

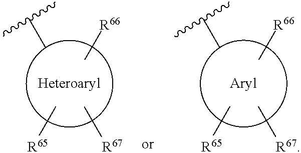

40. The compound of embodiment D39 wherein the compound is

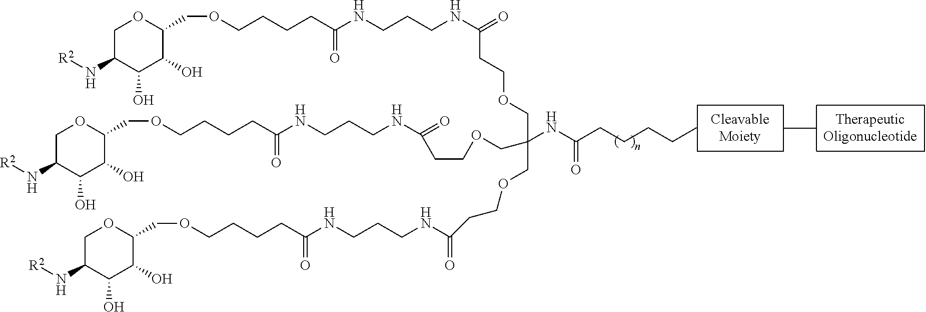

or a pharmaceutically acceptable salt thereof.
41. The compound of embodiment D39 wherein the compound is

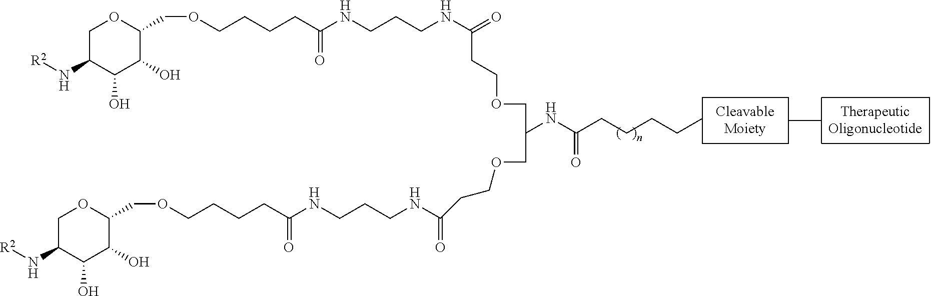

or a pharmaceutically acceptable salt thereof.
42. The compound of embodiment D39 wherein the compound is

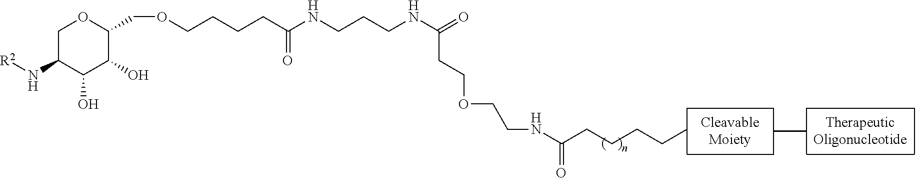

or a pharmaceutically acceptable salt thereof.
43. The compound of embodiment D1, wherein the compound is selected from the group consisting of:

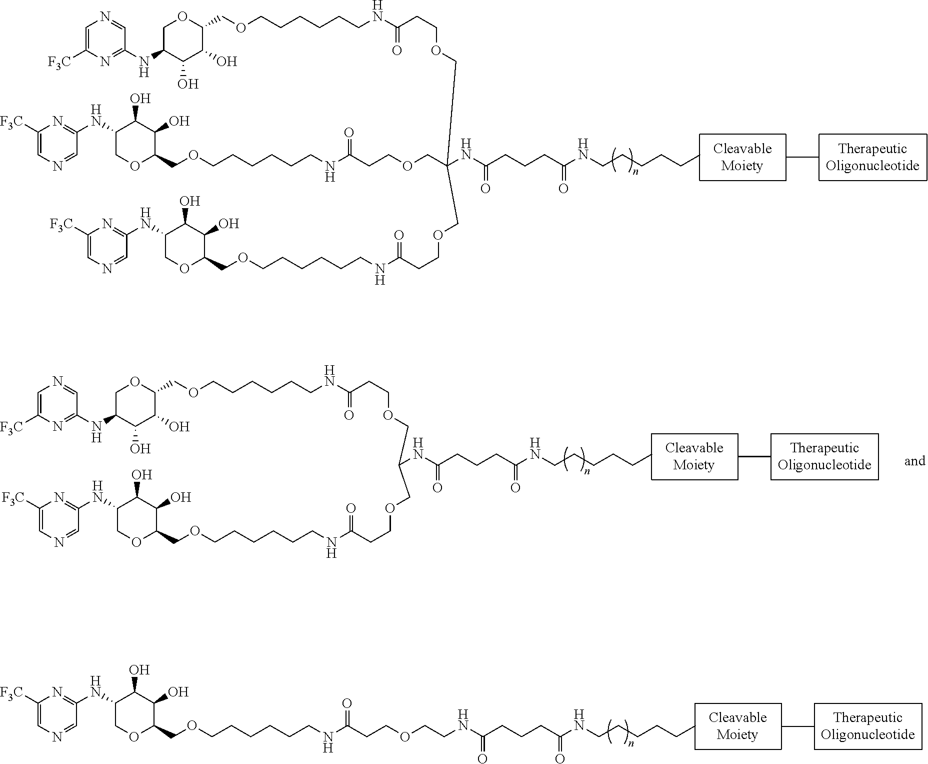

or a pharmaceutically acceptable salt thereof,
wherein
n is 0, 1, 2, 3, 4, 5, 6, 7, 8, 9, or 10.
44. The compound of embodiment D43 wherein the compound is of Formula

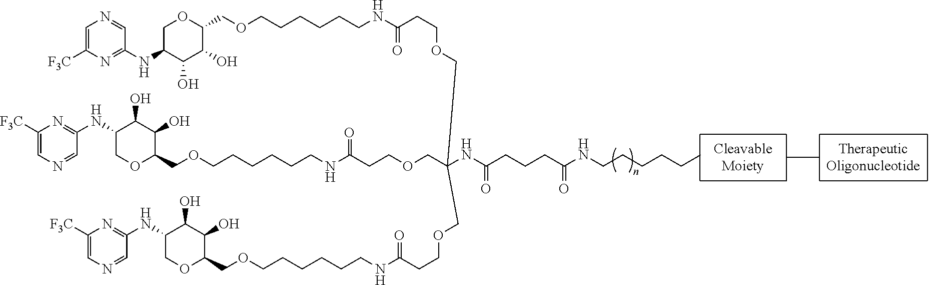

or a pharmaceutically acceptable salt thereof.

45. The compound of embodiment D43 wherein the compound is of Formula

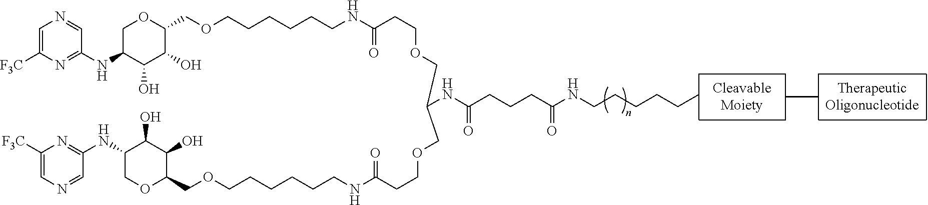

or a pharmaceutically acceptable salt thereof.
46. The compound of embodiment D43 wherein the compound is of Formula

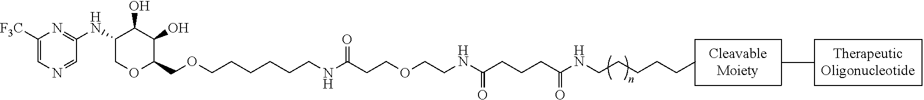

or a pharmaceutically acceptable salt thereof.
47. The compound of embodiment D1, wherein the compound is selected from the group consisting of

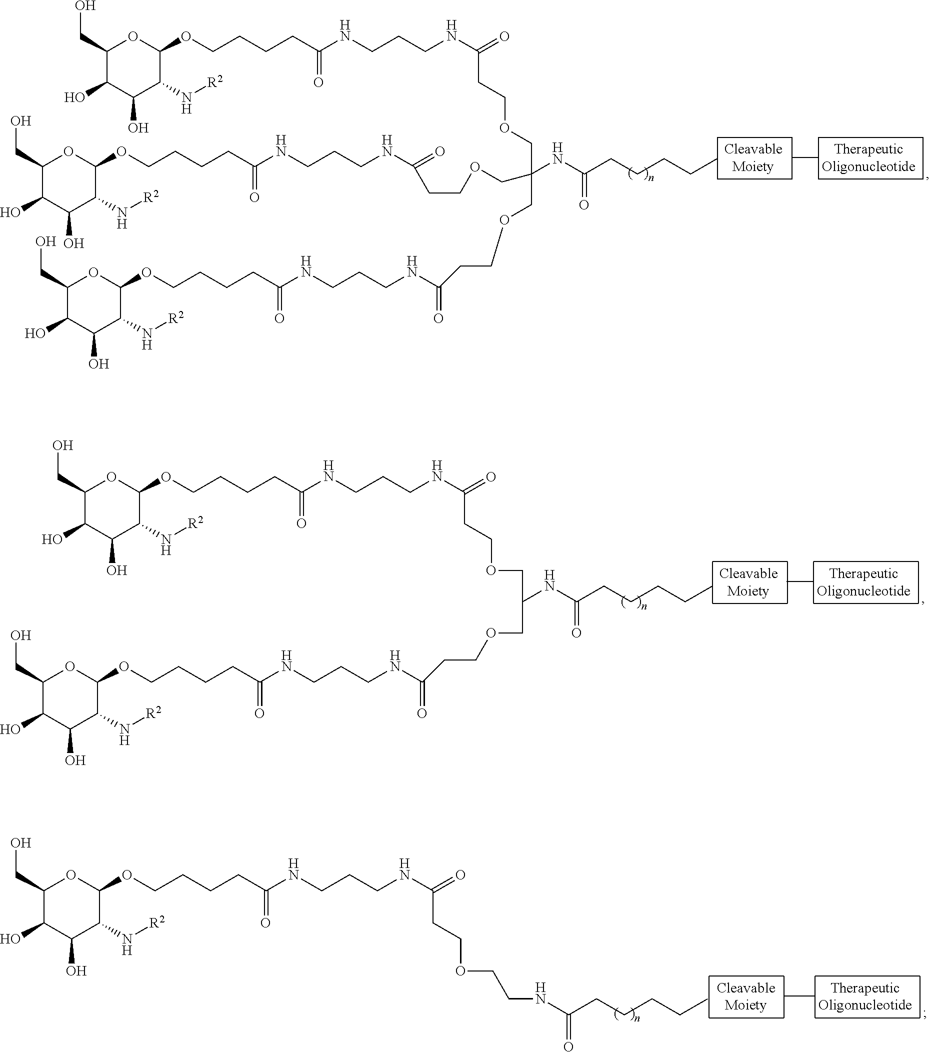

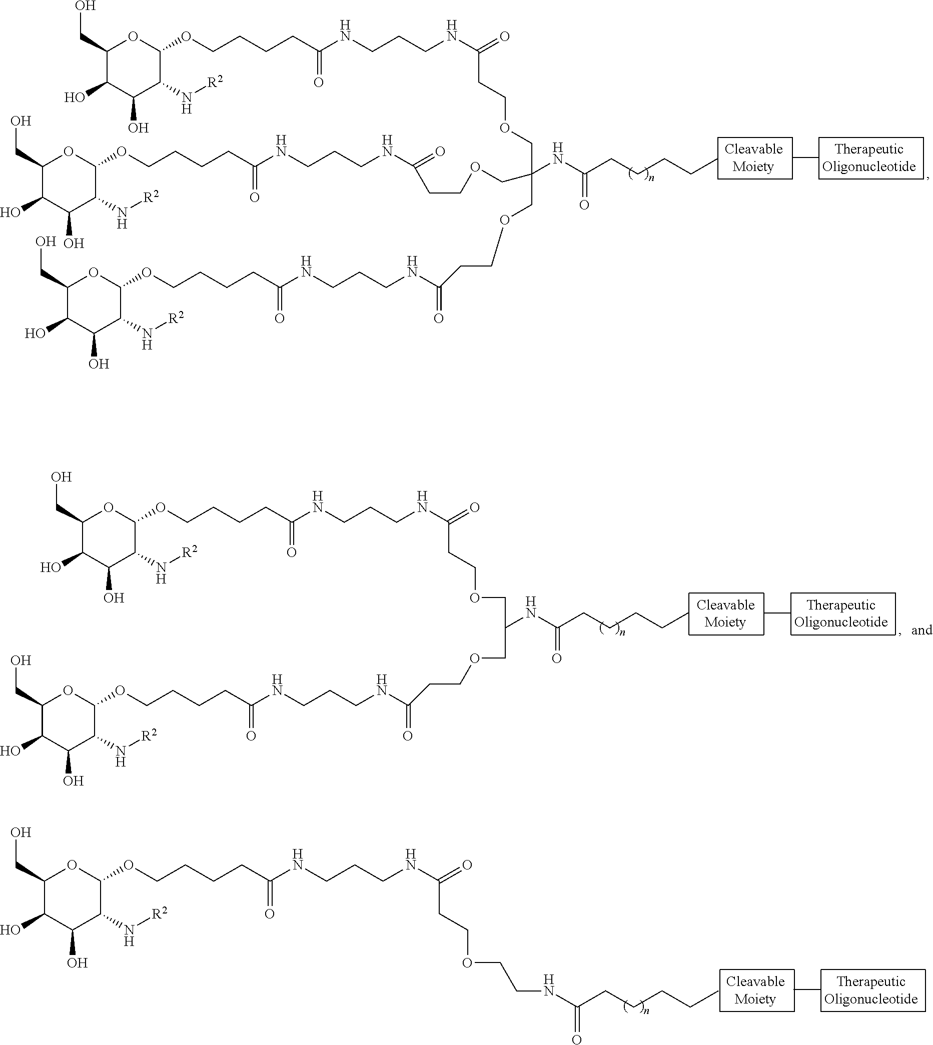

or a pharmaceutically acceptable salt thereof,
wherein
n is 0, 1, 2, 3, 4, 5, 6, 7, 8, 9, or 10; and
R² is

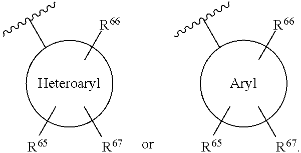

48. The compound of embodiment D1, wherein the compound is selected from the group consisting of:

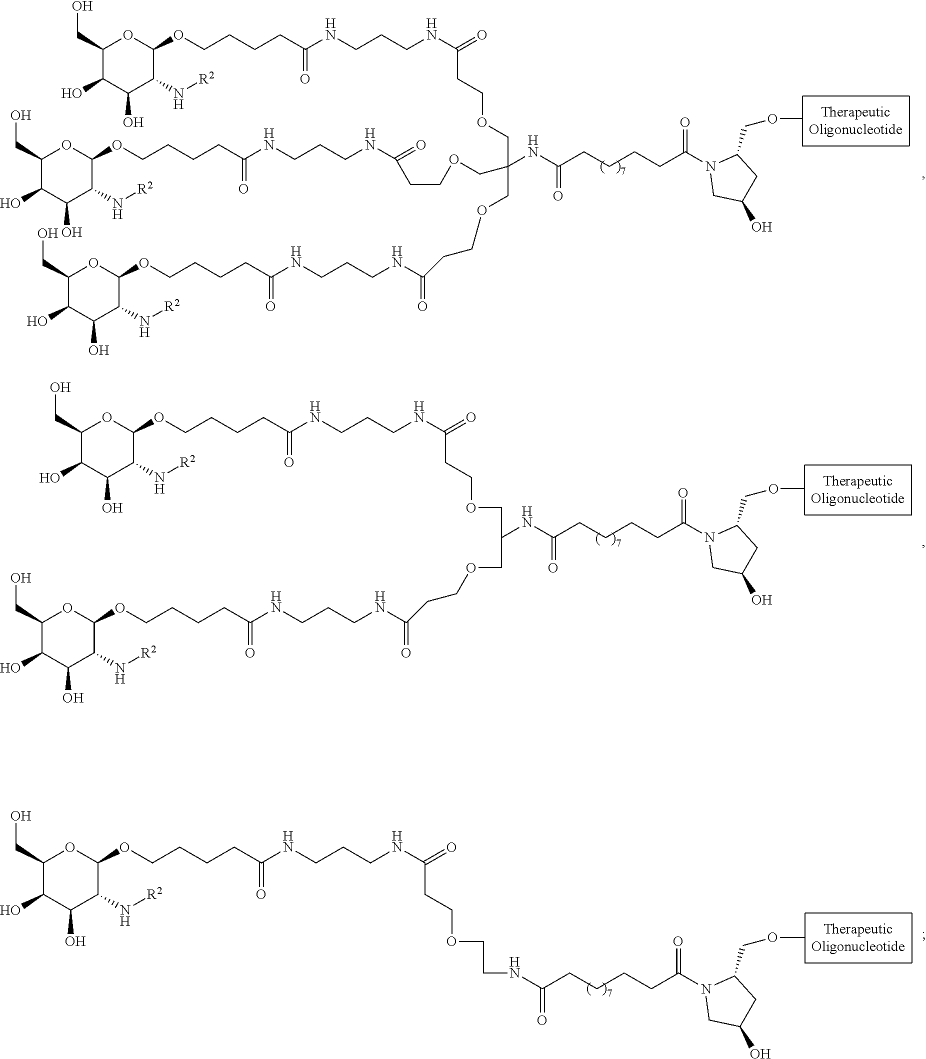

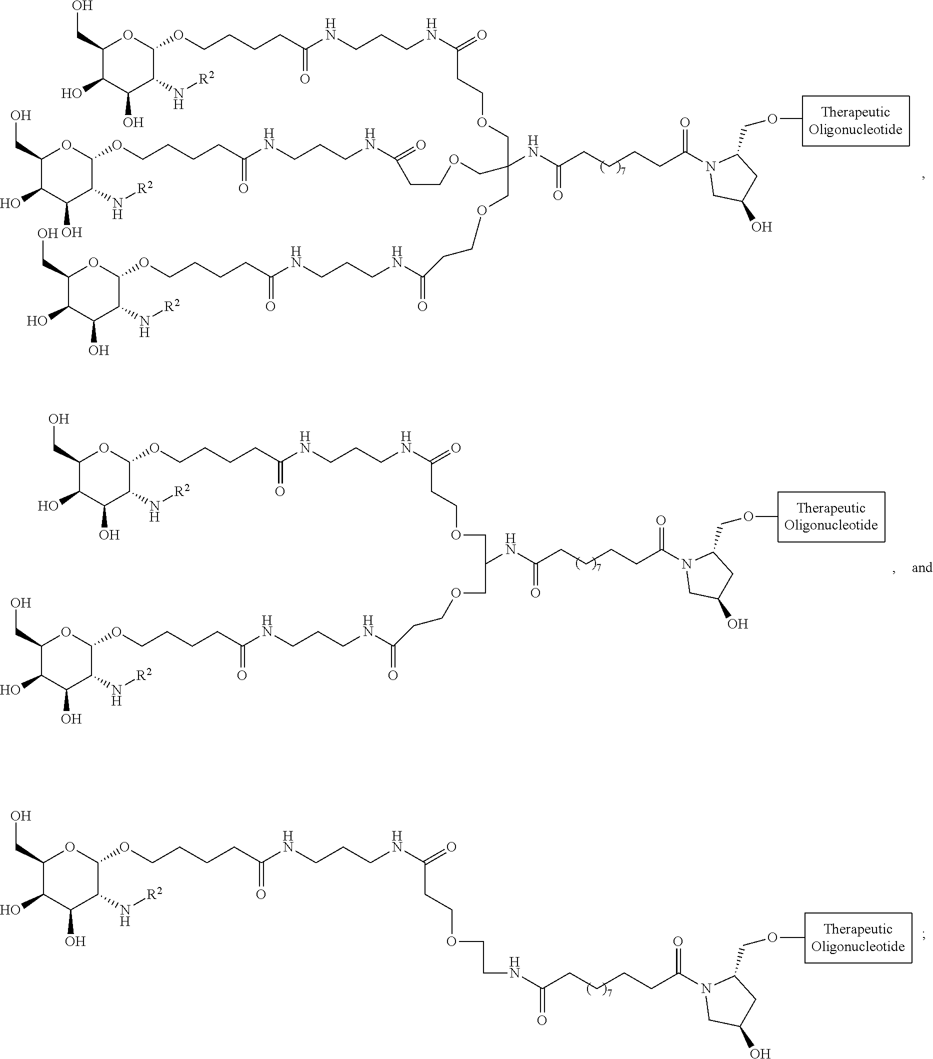

or a pharmaceutically acceptable salt thereof,
wherein
n is 0, 1, 2, 3, 4, 5, 6, 7, 8, 9, or 10; and
R² is

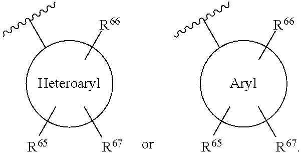

49. The compound of any one of embodiments D1-D48, wherein Therapeutic Oligonucleotide is a nucleotide described in Table 1.
50. The compound of any one of embodiments D1-D48, wherein Therapeutic Oligonucleotide is a nucleotide that is at least 80% identical to a nucleotide described in Table 1.
51. The compound of any one of embodiments D1-D48, wherein Therapeutic Oligonucleotide is a nucleotide that is at least 85% identical to a nucleotide described in Table 1.
52. The compound of any one of embodiments D1-D48, wherein Therapeutic Oligonucleotide is a nucleotide that is at least 90% identical to a nucleotide described in Table 1.
53. The compound of any one of embodiments D1-D48, wherein Therapeutic Oligonucleotide is a nucleotide that is at least 95% identical to a nucleotide described in Table 1.
54. The compound of any one of embodiments D1-D48, wherein Therapeutic Oligonucleotide is a nucleotide that is at least 96% identical to a nucleotide described in Table 1.
55. The compound of any one of embodiments D1-D48, wherein Therapeutic Oligonucleotide is a nucleotide that is at least 97% identical to a nucleotide described in Table 1.
56. The compound of any one of embodiments D1-D48, wherein Therapeutic Oligonucleotide is a nucleotide that is at least 98% identical to a nucleotide described in Table 1.
57. The compound of any one of embodiments D1-D48, wherein Therapeutic Oligonucleotide is a nucleotide that is at least 99% identical to a nucleotide described in Table 1.
58. The compound of any one of embodiments D1-D57, wherein the Therapeutic Oligonucleotide is Therapeutic Oligonucleotide 1.
59. The compound of any one of embodiments D1-D57, wherein the Therapeutic Oligonucleotide is Therapeutic Oligonucleotide 2.
60. The compound of any one of embodiments D1-D57, wherein the Therapeutic Oligonucleotide is Therapeutic Oligonucleotide 3.
61. The compound of any one of embodiments D1-D57, wherein the Therapeutic Oligonucleotide is Therapeutic Oligonucleotide 4.
62. The compound of any one of embodiments D1-D57, wherein the Therapeutic Oligonucleotide is Therapeutic Oligonucleotide 5.
63. The compound of any one of embodiments D1-D57, wherein the Therapeutic Oligonucleotide is Therapeutic Oligonucleotide 6.
64. The compound of any one of embodiments D1-D57, wherein the Therapeutic Oligonucleotide is Therapeutic Oligonucleotide 7.
65. The compound of any one of embodiments D1-D57, wherein the Therapeutic Oligonucleotide is Therapeutic Oligonucleotide 8.
66. The compound of any one of embodiments D1-D57, wherein the Therapeutic Oligonucleotide is Therapeutic Oligonucleotide 9.
67. The compound of any one of embodiments D1-D57, wherein the Therapeutic Oligonucleotide is Therapeutic Oligonucleotide 10.
68. The compound of any one of embodiments D1-D57, wherein the Therapeutic Oligonucleotide is Therapeutic Oligonucleotide 11.
69. The compound of any one of embodiments D1-D57, wherein the Therapeutic Oligonucleotide is Therapeutic Oligonucleotide 12.
70. The compound of any one of embodiments D1-D57, wherein the Therapeutic Oligonucleotide is Therapeutic Oligonucleotide 13.
71. The compound of any one of embodiments D1-D57, wherein the Therapeutic Oligonucleotide is Therapeutic Oligonucleotide 14.
72. The compound of any one of embodiments D1-D57, wherein the Therapeutic Oligonucleotide is Therapeutic Oligonucleotide 15.
73. The compound of any one of embodiments D1-D57, wherein the Therapeutic Oligonucleotide is Therapeutic Oligonucleotide 16.
74. The compound of any one of embodiments D1-D57, wherein the Therapeutic Oligonucleotide is Therapeutic Oligonucleotide 17.
75. The compound of any one of embodiments D1-D57, wherein the Therapeutic Oligonucleotide is Therapeutic Oligonucleotide 18.
76. The compound of any one of embodiments D1-D57, wherein the Therapeutic Oligonucleotide is Therapeutic Oligonucleotide 19.
77. The compound of any one of embodiments D1-D57, wherein the Therapeutic Oligonucleotide is Therapeutic Oligonucleotide 20.
78. The compound of any one of embodiments D1-D57, wherein the Therapeutic Oligonucleotide is Therapeutic Oligonucleotide 21.
79. The compound of any one of embodiments D1-D57, wherein the Therapeutic Oligonucleotide is Therapeutic Oligonucleotide 22.
80. The compound of any one of embodiments D1-D57, wherein the Therapeutic Oligonucleotide is Therapeutic Oligonucleotide 23.
81. The compound of any one of embodiments D1-D57, wherein the Therapeutic Oligonucleotide is Therapeutic Oligonucleotide 24.
82. The compound of any one of embodiments D1-D57, wherein the Therapeutic Oligonucleotide is Therapeutic Oligonucleotide 25.
83. The compound of any one of embodiments D1-D57, wherein the Therapeutic Oligonucleotide is Therapeutic Oligonucleotide 26.
84. The compound of any one of embodiments D1-D57, wherein the Therapeutic Oligonucleotide is Therapeutic Oligonucleotide 27.
85. The compound of any one of embodiments D1-D57, wherein the Therapeutic Oligonucleotide is Therapeutic Oligonucleotide 28.
86. The compound of any one of embodiments D1-D57, wherein the Therapeutic Oligonucleotide is Therapeutic Oligonucleotide 29.
87. A compound selected from

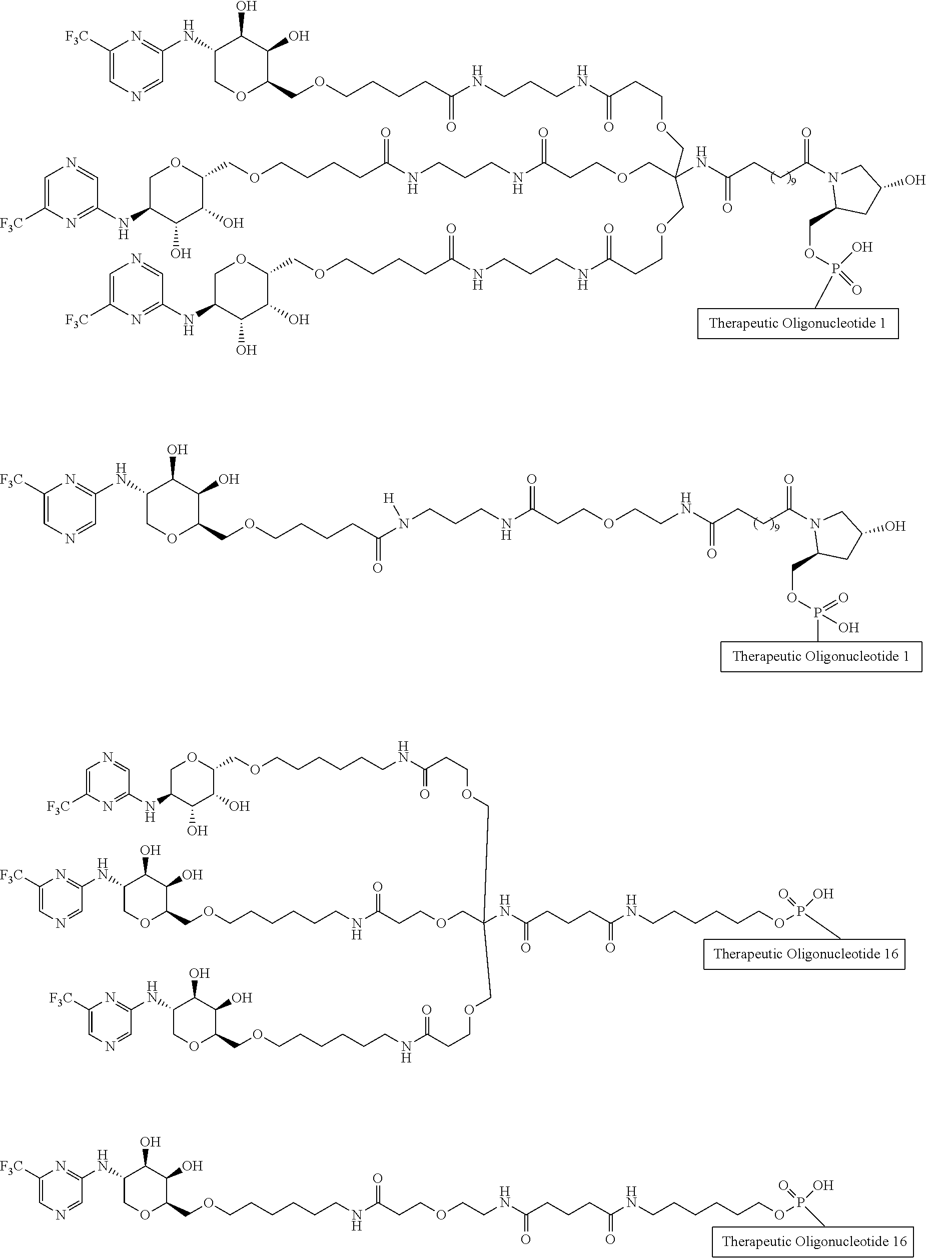

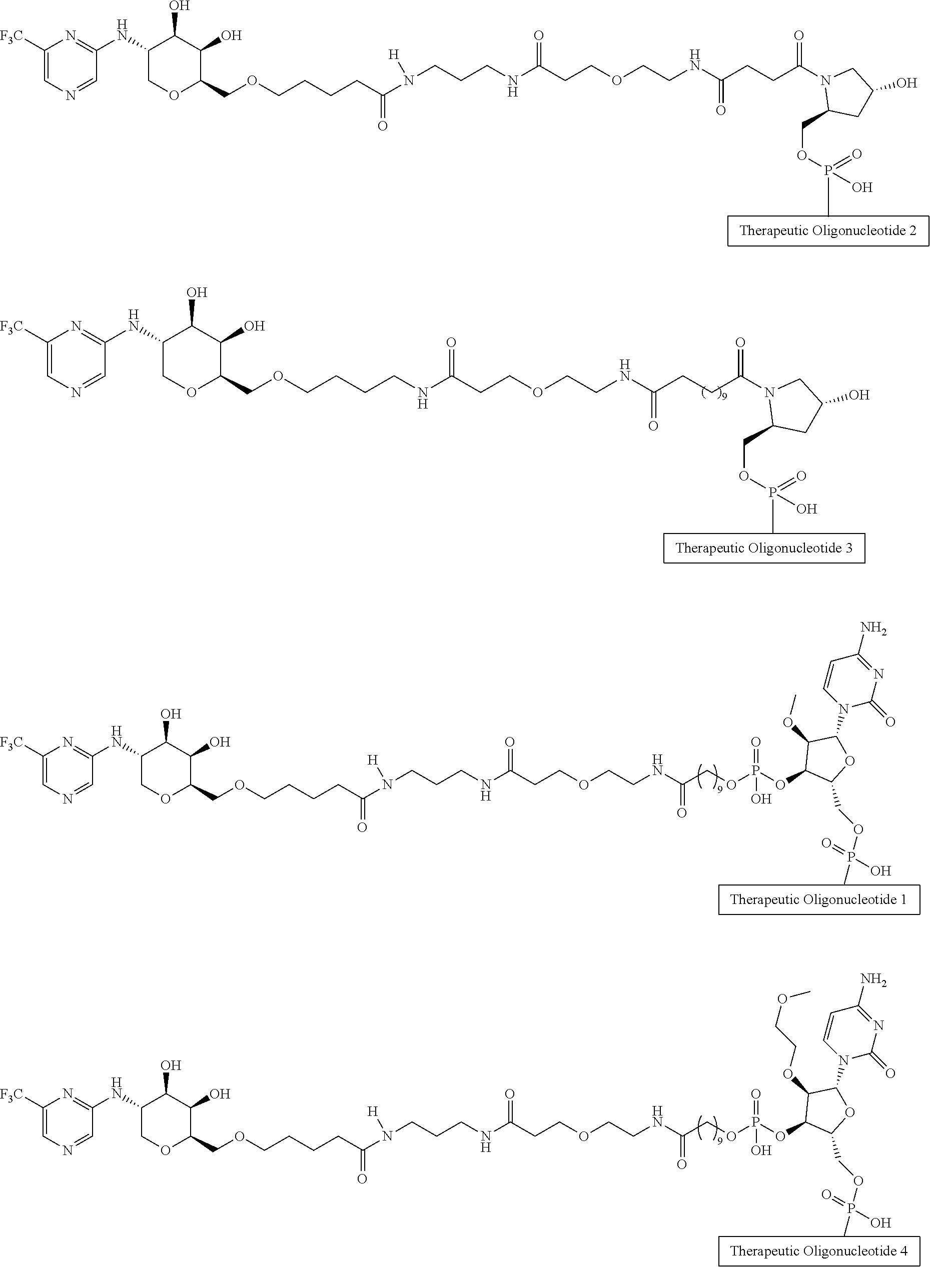

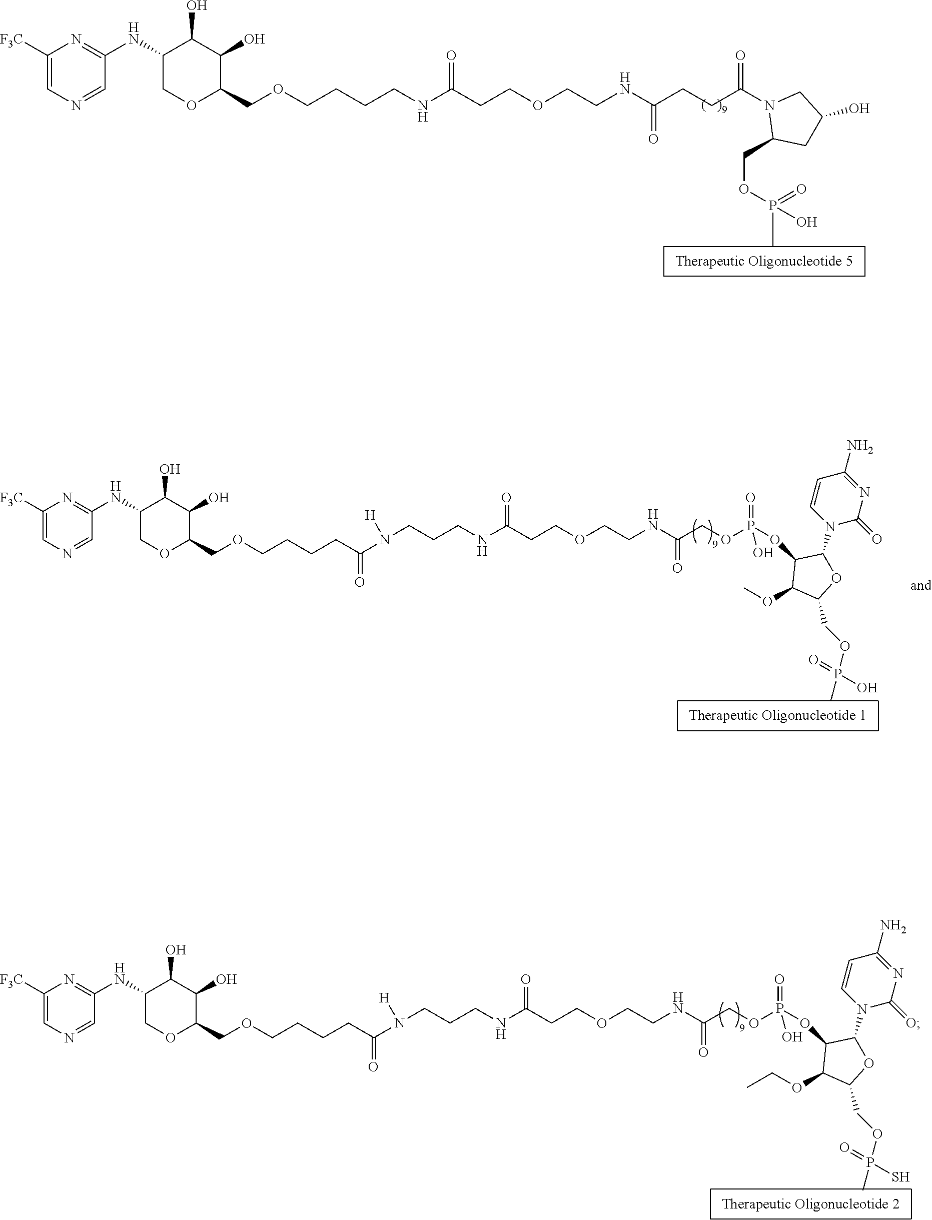

or a pharmaceutically acceptable salt thereof.
88. A pharmaceutical composition comprising a compound of any one of embodiments D1-D87 or a pharmaceutically acceptable salt thereof and a pharmaceutically acceptable excipient.
89. A method for the treatment of a disorder comprising administering an effective amount of a compound of any one of embodiments D1-D87 or a pharmaceutically acceptable salt thereof or a pharmaceutical composition thereof to a human patient.
90. The method of embodiment D89, wherein the disorder responds to treatment with the Therapeutic Oligonucleotide.
91. The method of embodiment D89 or D90, wherein the disorder is a liver associated disorder.
92. The method of embodiment D89 or D90, wherein the disorder is selected from acute hepatic porphyria (AHP), hyperoxaluria type 1 (PH1), hypercholesterolemia, hemophilia, a complement mediated disorder, HBV, hypertension, nonalcoholic steatohepatitis (NASH), a cardiovascular disease, hyperoxaluria type 2, hyperoxaluria type 3, β-thalassemia, immunoglobulin A nephropathy (IgAN), angioedema, myelodysplastic disorder, and treatment resistant hypertension.

### V. METHODS OF TREATMENT

In certain aspects, the Therapeutic Oligonucleotide delivering compound of the current invention can be administered in an effective amount for the treatment a disorder which can be treated with the Therapeutic Oligonucleotide, for example a liver disorder.

In some embodiments, provided are methods of treating a disorder comprising administering to a subject in need thereof an effective amount of a Therapeutic Oligonucleotide delivering compound as described herein or a pharmaceutically acceptable salt thereof. In some embodiments, the disorder is selected from acute coronary syndrome, acute hepatic porphyria (AHP), acute intermittent porphyria, acute porphyria, age-related macular degeneration (AMD), ALA dehydratase deficient porphyria (ADP), amyloidosis, aortic stenosis, atherosclerosis, atherosclerotic cardiovascular disease (ASCVD), atypical hemolytic uremic syndrome, autoimmune hepatitis, Berger disease, beta thalassemia intermedia, biliary atresia, cardiovascular disease, carotid artery plaque, chronic heart failure with reduced ejection fraction, chronic hepatitis A, chronic hepatitis A virus (cHAV) infection, chronic hepatitis B, chronic hepatitis B virus (cHBV) infection, chronic hepatitis C, chronic hepatitis C virus (cHCV) infection, chronic hepatitis D, chronic hepatitis D virus (cHDV) infection, cirrhosis, coronary artery disease, elevated lipoprotein(a) (Lp(a)), elevated serum lipoprotein(a) (Lp(a)), elevated triglycerides (TG), end-stage renal disease, end-stage renal disease requiring hemodialysis, generalized myasthenia gravis, geographic atrophy, hemochromatosis, hemophilia, hemophilia A, hemophilia B, hepatic impairment, hepatitis A, hepatitis B, hepatitis C, hepatitis D, hereditary amyloidosis, hereditary angioedema, heterozygous familial hypercholesterolemia (HeFH), homozygous familial hypercholesterolemia (HoFH), hypercholesterolemia, hyperlipidemia, hyperlipoproteinemia (a), hypertension, hypertriglyceridemia, IgA glomerulonephritis, IgA nephropathy (IgAN), Inflammation, kidney disease, lipoproteinemia, low risk myelodysplastic syndrome, macular degeneration, metabolic-associated fatty liver disease (MAFLD), mild hypertension, mixed dyslipidemia, nonalcoholic fatty liver disease (NAFD), nonalcoholic steatohepatitis (NASH), nonalcoholic steatohepatitis (NASH) with fibrosis, non-transfusion-dependent thalassemia, paroxysmal nocturnal hemoglobinuria, phlebotomy dependent polycythemia vera, polycythemia vera, primary biliary cholangitis, primary hypercholesterolemia, primary hyperoxaluria, primary hyperoxaluria Type 1 (PH1), primary hyperoxaluria Type 2 (PH2), primary hyperoxaluria Type 3 (PH3), primary IgA nephropathy, primary sclerosing cholangitis, renal impairment, resistant hypertension, thalassemia, type 2 diabetes, urologic disease, Wilson's disease, or very-low risk myelodysplastic syndrome.

In certain aspects, the disorder treated by a Therapeutic Oligonucleotide delivering compound described herein or a pharmaceutically acceptable salt thereof of the present invention is selected from acute hepatic porphyria (AHP), hyperoxaluria type 1 (PH1), hypercholesterolemia, hemophilia, a complement mediated disorder, HBV, hypertension, nonalcoholic steatohepatitis (NASH), a cardiovascular disease, hyperoxaluria type 2, hyperoxaluria type 3, β-thalassemia, immunoglobulin A nephropathy (IgAN), angioedema, myelodysplastic disorder, and treatment resistant hypertension.

In some embodiments, provided are methods of treating a liver disorder comprising administering to a subject in need thereof an effective amount of a Therapeutic Oligonucleotide delivering compound described herein or a pharmaceutically acceptable salt thereof as described herein. In some embodiments, the liver disorder is selected from acute hepatic porphyria (AHP), acute intermittent porphyria, acute porphyria, ALA dehydratase deficient porphyria (ADP), autoimmune hepatitis, beta thalassemia (β-thalassemia), beta thalassemia intermedia, biliary atresia, chronic hepatitis A, chronic hepatitis A virus (cHAV) infection, chronic hepatitis B, chronic hepatitis B virus (cHBV) infection, chronic hepatitis C, chronic hepatitis C virus (cHCV) infection, chronic hepatitis D, chronic hepatitis D virus (cHDV) infection, cirrhosis, hemochromatosis, hepatic impairment, hepatitis A, hepatitis B, hepatitis C, hepatitis D, hypercholesterolemia, IgA nephropathy (IgAN), metabolic-associated fatty liver disease (MAFLD), nonalcoholic fatty liver disease (NAFD), nonalcoholic steatohepatitis (NASH), nonalcoholic steatohepatitis (NASH) with fibrosis, non-transfusion-dependent thalassemia, primary biliary cholangitis, primary sclerosing cholangitis, thalassemia, or Wilson's disease.

In certain aspects a compound of the present invention is administered prophylactically. For example, when the Therapeutic Oligonucleotide is an antithrombin lowering agent such as fitusiran the compound of the present invention may be administered prophylactically to decrease the risk of excessive bleeding events.

In some embodiments, the present disclosure provides a method to treat a disorder in a subject in need thereof comprising administering to the subject an effective amount of a Therapeutic Oligonucleotide delivering compound comprising the nucleotide component of givosiran. Givosiran is an siRNA that targets 5-aminolevulinate synthetase (ALASI). In some embodiments, the disorder is selected from acute porphyria, acute hepatic porphyria (AHP), ALA dehydratase deficient porphyria (ADP), or acute intermittent porphyria.

In some embodiments, the present disclosure provides a method to treat a disorder in a subject in need thereof comprising administering to the subject an effective amount of a Therapeutic Oligonucleotide delivering compound comprising the nucleotide component of lumasiran. Lumasiran is an siRNA that targets HAOl. In some embodiments, the disorder is primary hyperoxaluria. In some embodiments, the primary hyperoxaluria is primary hyperoxaluria Type 1 (PH1). In some embodiments, the primary hyperoxaluria is primary hyperoxaluria Type 2 (PH2). In some embodiments, the primary hyperoxaluria is primary hyperoxaluria Type 3 (PH3).

In some embodiments, the present disclosure provides a method to treat a disorder in a subject in need thereof comprising administering to the subject an effective amount of a Therapeutic Oligonucleotide delivering compound comprising the nucleotide component of inclisiran. Inclisiran (ALN-60212) is an siRNA that targets PCSK9. In some embodiments, the disorder is selected from atherosclerotic cardiovascular disease (ASCVD), hypercholesterolemia, primary hypercholesterolemia, heterozygous familial hypercholesterolemia (HeFH), homozygous familial hypercholesterolemia (HoFH), renal impairment, mixed dyslipidemia, acute coronary syndrome, type 2 diabetes, atherosclerosis, hyperlipidemia, coronary artery disease, or carotid artery plaque.

In some embodiments, the present disclosure provides a method to treat a disorder in a subject in need thereof comprising administering to the subject an effective amount of a Therapeutic Oligonucleotide delivering compound comprising the nucleotide component of fitusiran. Fitusiran, also known as ALN-57213, is an siRNA that targets SERPINCl. In some embodiments, the disorder is hemophilia. In some embodiments, the hemophilia is hemophilia A. In some embodiments, the hemophilia is hemophilia B.

In some embodiments, the present disclosure provides a method to treat a disorder in a subject in need thereof comprising administering to the subject an effective amount of a Therapeutic Oligonucleotide delivering compound comprising the nucleotide component of cemdisiran. Cemdisiran (AD062643) is an siRNA that targets complement C₅. In some embodiments, the disorder is selected from atypical hemolytic uremic syndrome, Berger disease, IgA glomerulonephritis, IgA nephropathy, paroxysmal nocturnal hemoglobinuria, or myasthenia gravis.

In some embodiments, the present disclosure provides a method to treat a disorder in a subject in need thereof comprising administering to the subject an effective amount of a Therapeutic Oligonucleotide delivering compound comprising the nucleotide component of VIR-2218 (ALN-HBV02). In some embodiments, the disorder is selected from chronic hepatitis B virus (cHBV) infection, chronic hepatitis D virus (cHDV) infection, hepatitis, hepatitis B, chronic hepatitis B, hepatitis D, chronic hepatitis D, renal impairment, hepatic impairment, or cirrhosis.

In some embodiments, the present disclosure provides a method to treat a disorder in a subject in need thereof comprising administering to the subject an effective amount of a Therapeutic Oligonucleotide delivering compound comprising the nucleotide component of zilebesiran (ALN-AGT). In some embodiments, the disorder is hypertension.

In some embodiments, the present disclosure provides a method to treat a disorder in a subject in need thereof comprising administering to the subject an effective amount of a Therapeutic Oligonucleotide delivering compound comprising the nucleotide component of ALN-HSD. In some embodiments, the disorder is selected from nonalcoholic steatohepatitis (NASH) or nonalcoholic steatohepatitis (NASH) with fibrosis.

In some embodiments, the present disclosure provides a method to treat a disorder in a subject in need thereof comprising administering to the subject an effective amount of a Therapeutic Oligonucleotide delivering compound comprising the nucleotide component of olpasiran. Olpasiran is an siRNA that targets lipoprotein(a) (Lp(a). In some embodiments, the disorder is selected from renal impairment, elevated serum lipoprotein(a) (Lp(a)), atherosclerotic cardiovascular disease (ASCVD), or cardiovascular disease.

In some embodiments, the present disclosure provides a method to treat a disorder in a subject in need thereof comprising administering to the subject an effective amount of a Therapeutic Oligonucleotide delivering compound comprising the nucleotide component of nedosiran. Nedosiran is an siRNA that targets LDHA. In some embodiments, the disorder is selected from primary hyperoxaluria, primary hyperoxaluria Type 1 (PH1), primary hyperoxaluria Type 2 (PH2), primary hyperoxaluria Type 3 (PH3), end stage renal disease, kidney disease, or urologic disease.

In some embodiments, the present disclosure provides a method to treat a disorder in a subject in need thereof comprising administering to the subject an effective amount of a Therapeutic Oligonucleotide delivering compound comprising the nucleotide component of xalnesiran. In some embodiments, the disorder is selected from hepatitis, hepatitis B, chronic hepatitis B, or chronic hepatitis B virus (cHBV) infection.

In some embodiments, the present disclosure provides a method to treat a disorder in a subject in need thereof comprising administering to the subject an effective amount of a Therapeutic Oligonucleotide delivering compound comprising the nucleotide component of zerlasiran (SLN360). In some embodiments, the disorder is selected from elevated lipoprotein(a) (Lp(a)) or atherosclerotic cardiovascular disease (ASCVD).

In some embodiments, the present disclosure provides a method to treat a disorder in a subject in need thereof comprising administering to the subject an effective amount of a Therapeutic Oligonucleotide delivering compound comprising the nucleotide component of pelacarsen. Pelacarsen (IONIC-APO(a)-LRX, ISIS-681257) is an antisense oligonucleotide that targets apolipoprotein A. In some embodiments, the disorder is selected from hepatic impairment, hyperlipoproteinemia (a), acute coronary syndrome, lipoprotenemia, inflammation, aortic stenosis, elevated lipoprotein(a) (Lp(a)), or cardiovascular disease.

In some embodiments, the present disclosure provides a method to treat a disorder in a subject in need thereof comprising administering to the subject an effective amount of a Therapeutic Oligonucleotide delivering compound comprising the nucleotide component of IONIS-APOCIII-LRx. In some embodiments, the disorder is selected from elevated triglycerides (TG), hypertriglyceridemia, or cardiovascular disease.

In some embodiments, the present disclosure provides a method to treat a disorder in a subject in need thereof comprising administering to the subject an effective amount of a Therapeutic Oligonucleotide delivering compound comprising the nucleotide component of IONIS-FB-LRx. In some embodiments, the disorder is selected from primary IgA nephropathy, geographic atrophy, macular degeneration, or age-related macular degeneration (AMD).

In some embodiments, the present disclosure provides a method to treat a disorder in a subject in need thereof comprising administering to the subject an effective amount of a Therapeutic Oligonucleotide delivering compound comprising the nucleotide component of fesomersen. In some embodiments, the disorder is end-stage renal disease requiring hemodialysis.

In some embodiments, the present disclosure provides a method to treat a disorder in a subject in need thereof comprising administering to the subject an effective amount of a Therapeutic Oligonucleotide delivering compound comprising the nucleotide component of donidalorsen. Donidalorsen (ISIS-721744) is a plasma kallikrein inhibitor. In some embodiments, the disorder is hereditary angioedema.

In some embodiments, the present disclosure provides a method to treat a disorder in a subject in need thereof comprising administering to the subject an effective amount of a Therapeutic Oligonucleotide delivering compound comprising the nucleotide component of tonlamarsen. In some embodiments, the disorder is resistant hypertension.

In some embodiments, the present disclosure provides a method to treat a disorder in a subject in need thereof comprising administering to the subject an effective amount of a Therapeutic Oligonucleotide delivering compound comprising the nucleotide component of ARO-HBV. In some embodiments, the disorder is hepatitis B.

In some embodiments, the present disclosure provides a method to treat a disorder in a subject in need thereof comprising administering to the subject an effective amount of a Therapeutic Oligonucleotide delivering compound comprising the nucleotide component of IONIS-AGT-LRx. In some embodiments, the disorder is selected from hypertension, chronic heart failure with reduced ejection fraction, or mild hypertension.

In some embodiments, the present disclosure provides a method to treat a disorder in a subject in need thereof comprising administering to the subject an effective amount of a Therapeutic Oligonucleotide delivering compound comprising the nucleotide component of AB-729. AB-729 is an anti-miRNA (antimir) that targets hepatitis B surface antigen (HBsAg). In some embodiments, the disorder is selected from hepatitis B, chronic hepatitis B, chronic hepatitis B virus (cHBV) infection, hepatitis D, chronic hepatitis D, or chronic hepatitis D virus (cHDV) infection.

In some embodiments, the present disclosure provides a method to treat a disorder in a subject in need thereof comprising administering to the subject an effective amount of a Therapeutic Oligonucleotide delivering compound comprising the nucleotide component of SLN-124. In some embodiments, the disorder is selected from polycythemia vera, low risk myelodysplastic syndrome, very-low risk myelodysplastic syndrome, or non-transfusion-dependent thalassemia.

### VI. COMBINATION TREATMENT

In certain aspects, a treatment regimen is provided comprising the administration to a subject in need thereof of an effective amount of a Therapeutic Oligonucleotide delivering compound of the present invention or a pharmaceutically acceptable composition, salt, isotopic analog, or prodrug thereof in combination or in alternation with at least one additional therapeutic agent for the treatment of a disorder that can be treated with the Therapeutic Oligonucleotide. The combinations and/or alternations disclosed herein can be administered for beneficial, additive, or synergistic effect in the treatment of disorders, for example, a disorder of the liver.

In some embodiments the combination therapy comprises administering a caprylic acid derivative. Non-limiting examples of caprylic acid derivatives are provided in the section “CAPRYLIC ACID DERIVATIVES.” In certain aspects the combination therapy comprising a caprylic acid derivative and a compound of the present invention is delivered orally.

In some embodiments, a treatment regimen is provided for the treatment of a disorder comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, at least one of the additional therapeutic agents is a non-nucleoside reverse transcriptase inhibitor. In some embodiments, at least one of the additional therapeutic agents is a nucleoside or nucleotide reverse transcriptase inhibitor. In some embodiments, at least one of the additional therapeutic agents is a protease inhibitor. In some embodiments, at least one of the additional therapeutic agents is an integrase inhibitor. In some embodiments, at least one of the additional therapeutic agents is an entry or fusion inhibitor. In some embodiments, at least one of the additional therapeutic agents is selected from efavirenz, rilpivirine, doravirine, abacavir, tenofovir, emtricitabine, lamivudine, zidovudine, atazanavir, darunavir, lopinavir, ritonavir, bictegravir, raltegravir, dolutegravir, enfuvirtide, and maraviroc.

In some embodiments, a treatment regimen is provided for the treatment of a porphyria comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the porphyria is selected from acute hepatic porphyria (AHP), acute intermittent porphyria, acute porphyria, or ALA dehydratase deficient porphyria (ADP). In some embodiments, the at least one additional therapeutic agent is hormone therapy. In some embodiments, the at least one additional therapeutic agent is gonadotropin-releasing hormone (GnRH). In some embodiments, the at least one additional therapeutic agent is hemin.

In some embodiments, a treatment regimen is provided for the treatment of amyloidosis comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the at least one additional therapeutic agent is selected from anti-angiogenesis therapy, monoclonal antibodies, proteasome inhibitors, or a combination thereof. In some embodiments, the at least one additional therapeutic agent is selected from lenalidomide (Revlimid), pomalidomide (Pomalyst), or thalidomide (Synovir, Thalomid).

In some embodiments, a treatment regimen is provided for the treatment of thalessemia comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the thalassemia is selected from beta thalassemia intermedia, non-transfusion-dependent thalassemia, or beta thalassemia. In some embodiments, the at least one additional therapeutic agent is iron chelation therapy. In some embodiments, the at least one additional therapeutic agent is selected from deferasirox, deferiprone deferoxamine, luspatercept (Reblozyl), or hydroxyurea.

In some embodiments, a treatment regimen is provided for the treatment of hepatitis comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the hepatitis is mediated by a virus selected from hepatitis A virus (HAV), hepatitis B virus (HBV), hepatitis C virus (HCV), or hepatitis D virus (HDV). In some embodiments, the hepatitis is selected from chronic hepatitis A, chronic hepatitis A virus (cHAV) infection), chronic hepatitis B, chronic hepatitis B virus (cHBV) infection, chronic hepatitis C, chronic hepatitis C virus (cHCV) infection, chronic hepatitis D, chronic hepatitis D virus (cHDV) infection, hepatitis A, hepatitis B, hepatitis C, or hepatitis D.

In some embodiments, the hepatitis is hepatitis B. In some embodiments, the hepatitis is hepatitis B and the at least one additional therapeutic agent is an antiviral therapy. In some embodiments, the antiviral therapy is selected from tenofovir (Viread), entecavir (Baraclude), lamivudine (Epivir), adefovir (Hepsera), or telbivudine. In some embodiments, the hepatitis is hepatitis B and the at least one additional therapeutic agent is Interferon alfa-2b (Intron A). In some embodiments, the hepatitis B is chronic hepatitis B. In some embodiments, the hepatitis B is chronic hepatitis B virus (cHBV) infection.

In some embodiments, the hepatitis is hepatitis C. In some embodiments, the hepatitis is hepatitis C and the at least one additional therapeutic agent is direct-acting antivirals (DAA). In some embodiments, the DAA is selected from sofosbuvir, daclatasvir, ombitasvir, paritaprevir, ritonavir, dasabuvir, elbasvir, elbasvir, grazoprevir, glecaprevir, pibrentasvir, ledipasvir, or sofosbuvir. In some embodiments, the hepatitis C is chronic hepatitis C. In some embodiments, the hepatitis C is chronic hepatitis C virus (cHCV) infection.

In some embodiments, a treatment regimen is provided for the treatment of hypercholesterolemia comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the hypercholesterolemia is selected from heterozygous familial hypercholesterolemia (HeFH), homozygous familial hypercholesterolemia (HoFH), or primary hypercholesterolemia. In some embodiments, the at least one additional therapeutic agent is statin therapy. In some embodiments, the statin therapy is selected from atorvastatin, Fluvastatin, lovastatin, pitavastatin, pravastatin, rosuvastatin, simvastatin. In some embodiments, the at least one additional therapeutic agent is ezetimibe. In some embodiments, the at least one additional therapeutic agent is PCSK9 inhibitor therapy. In some embodiments, the PCSK9 inhibitor is selected from alirocumab or evolocumab.

In some embodiments, a treatment regimen is provided for the treatment of IgA nephropathy (IgAN) comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the at least one additional therapeutic agent is selected from angiotensin-converting enzyme (ACE) inhibitors or angiotensin receptor blockers (ARBs).

In some embodiments, a treatment regimen is provided for the treatment of hyperlipoproteinemia (a) comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the at least one additional therapeutic agent is selected from niacin, acetylsalicylic acid, or L-carnitine.

In some embodiments, a treatment regimen is provided for the treatment of primary biliary cholangitis comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, the at least one additional therapeutic agent is selected from ursodeoxycholic acid (UDCA), obeticholic acid, fibrates (Tricor), or budesonide.

In some embodiments, a treatment regimen is provided for the treatment of cirrhosis comprising the administration of a Therapeutic Oligonucleotide delivering compound of the present invention or a pharmaceutically acceptable composition, salt, isotopic analog, or prodrug thereof in combination or in alternation with at least one additional therapeutic agent. In some embodiments, at least one of the additional therapeutic agents is a glucocorticosteroid. In some embodiments, at least one of the additional therapeutic agents is an antioxidant. In some embodiments, at least one of the additional therapeutic agents is selected from pentoxifylline and infliximab.

### VII. CAPRYLIC ACID DERIVATIVES

In certain aspects the caprylic acid derivative is of Formula:

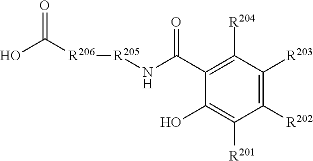

or a pharmaceutically acceptable salt thereof,
wherein:
   R²⁰¹, R²⁰², R²⁰³, and R²⁰⁴ are independently hydrogen, —OH, —NR⁶R⁷, halogen, C₁-C₄ alkyl, or C₁-C₄ alkoxy;
   R²⁰⁵ and R²⁰⁶ are independently selected from C₂-C₁₆ alkylene, C₂-C₁₆ alkenylene, and aryl, each of which is optionally substituted with 1, 2, or 3 substituents independently selected from R²⁰⁷;
   R²⁰⁷ is independently selected at each instance from the group consisting of alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³.
C1. In certain embodiments the caprylic acid derivative is of Formula:

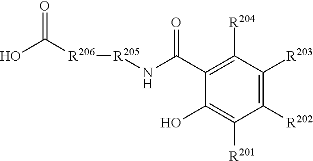

or a pharmaceutically acceptable salt thereof;
C2. The caprylic acid derivative of embodiment C1, wherein R²⁰¹ is hydrogen.
C3. The caprylic acid derivative of embodiment C1, wherein R²⁰¹ is —OH.
C4. The caprylic acid derivative of embodiment C1, wherein R²⁰¹ is —NR⁶R⁷.
C5. The caprylic acid derivative of embodiment C1, wherein R²⁰¹ is halogen.
C6. The caprylic acid derivative of embodiment C1, wherein R²⁰¹ is C₁-C₄ alkyl.
C7. The caprylic acid derivative of embodiment C1, wherein R²⁰¹ is C₁-C₄ alkoxy.
C8. The caprylic acid derivative of any one of embodiments C₁-C₇, wherein R²⁰² is hydrogen.
C9. The caprylic acid derivative of any one of embodiments C₁-C₇, wherein R²⁰² is —OH.
C10. The caprylic acid derivative of any one of embodiments C₁-C₇, wherein R²⁰² is —NR⁶R⁷.
C11. The caprylic acid derivative of any one of embodiments C₁-C₇, wherein R²⁰² is halogen.
C12. The caprylic acid derivative of any one of embodiments C₁-C₇, wherein R²⁰² is C₁-C₄ alkyl.
C13. The caprylic acid derivative of any one of embodiments C₁-C₇, wherein R²⁰² is C₁-C₄ alkoxy.
C14. The caprylic acid derivative of any one of embodiments C₁-C₁₃, wherein R²⁰³ is hydrogen.
C15. The caprylic acid derivative of any one of embodiments C₁-C₁₃, wherein R²⁰³ is —OH.
C16. The caprylic acid derivative of any one of embodiments C₁-C₁₃, wherein R²⁰³ is —NR⁶R⁷.
C17. The caprylic acid derivative of any one of embodiments C₁-C₁₃, wherein R²⁰³ is halogen.
C18. The caprylic acid derivative of any one of embodiments C₁-C₁₃, wherein R²⁰³ is C₁-C₄ alkyl.
C19. The caprylic acid derivative of any one of embodiments C₁-C₁₃, wherein R²⁰³ is C₁-C₄ alkoxy.
C20. The caprylic acid derivative of any one of embodiments C₁-C₁₉, wherein R²⁰⁴ is hydrogen.
C21. The caprylic acid derivative of any one of embodiments C₁-C₁₉, wherein R²⁰⁴ is —OH.
C22. The caprylic acid derivative of any one of embodiments C₁-C₁₉, wherein R²⁰⁴ is —NR⁶R⁷.
C23. The caprylic acid derivative of any one of embodiments C₁-C₁₉, wherein R²⁰⁴ is halogen.
C24. The caprylic acid derivative of any one of embodiments C₁-C₁₉, wherein R²⁰⁴ is C₁-C₄ alkyl.
C25. The caprylic acid derivative of any one of embodiments C₁-C₁₉, wherein R²⁰⁴ is C₁-C₄ alkoxy.
C26. The caprylic acid derivative of any one of embodiments C₁-C₂₅, wherein R²⁰⁵ is C₂-C₁₆ alkylene optionally substituted with 1, 2, or 3 substituents independently selected from R²⁰⁷.
C27. The caprylic acid derivative of any one of embodiments C₁-C₂₅, wherein R²⁰⁵ is C₂-C₁₆ alkenylene optionally substituted with 1, 2, or 3 substituents independently selected from R²⁰⁷.

C₂₈. The caprylic acid derivative of any one of embodiments C₁-C₂₅, wherein R²⁰⁵ is aryl optionally substituted with 1, 2, or 3 substituents independently selected from R²⁰⁷.
C29. The caprylic acid derivative of any one of embodiments C₁-C₂₈, wherein R²⁰⁶ is C₂-C₁₆ alkylene optionally substituted with 1, 2, or 3 substituents independently selected from R²⁰⁷.

C₃₀. The caprylic acid derivative of any one of embodiments C₁-C₂₈, wherein R²⁰⁶ is C₂-C₁₆ alkenylene optionally substituted with 1, 2, or 3 substituents independently selected from R²⁰⁷.

C₃₁. The caprylic acid derivative of any one of embodiments C₁-C₂₈, wherein R²⁰⁶ is aryl optionally substituted with 1, 2, or 3 substituents independently selected from R²⁰⁷.
C32. The caprylic acid derivative of any one of embodiments C₁-C₃₁, wherein R²⁰⁷ is hydrogen.
C33. The caprylic acid derivative of any one of embodiments C₁-C₃₁, wherein R²⁰⁷ is alkyl.
C34. The caprylic acid derivative of any one of embodiments C₁-C₃₁, wherein R²⁰⁷ is methyl.
C35. The caprylic acid derivative of any one of embodiments C₁-C₃₁, wherein R²⁰⁷ is halogen.
C36. The caprylic acid derivative of any one of embodiments C₁-C₃₁, wherein R²⁰⁷ is F.
C37. The caprylic acid derivative of any one of embodiments C₁-C₃₁, wherein R²⁰⁷ is C₁.
C38. The caprylic acid derivative of any one of embodiments C₁-C₃₁, wherein R²⁰⁷ is haloalkyl.
C39. The caprylic acid derivative of any one of embodiments C₁-C₃₁, wherein R²⁰⁷ is CF₃.
C40. The caprylic acid derivative of any one of embodiments C₁-C₃₁, wherein R²⁰⁷ is OMe.
C41. The caprylic acid derivative of any one of embodiments C₁-C₃₁, wherein R²⁰⁷ is OH.
C42. The caprylic acid derivative of any one of embodiments C₁-C₃₁, wherein R²⁰⁷ is NH₂.
C43. The caprylic acid derivative of any one of embodiments C₁-C₃₁, wherein R²⁰⁷ is NHMe.
C44. The caprylic acid derivative of any one of embodiments C₁-C₃₁, wherein R²⁰⁷ is NMe₂.
C45. The caprylic acid derivative of any one of embodiments C₁-C₃₁, wherein R²⁰⁷ is —C(O)R³.

### VIII. CLEAVABLE MOIETY

Cleavable Moiety is a chemical moiety that can be cleaved to release the oligonucleotide; in certain aspects Cleavable Moiety is a bond,

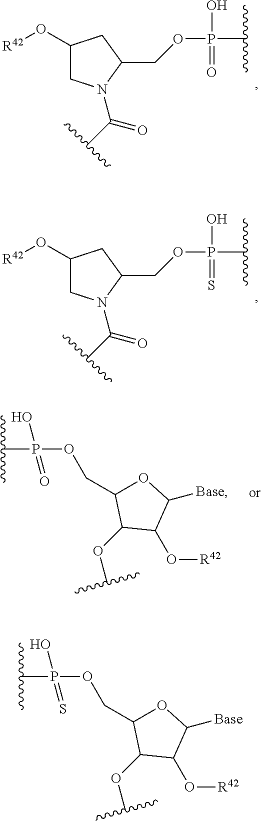

wherein the Therapeutic Oligonucleotide is attached to the —P—;

In certain embodiments Cleavable Moiety is a bond.

In certain embodiments Cleavable Moiety is

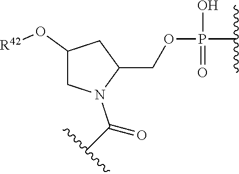

wherein the Therapeutic Oligonucleotide is attached to the —P—.

In certain embodiments Cleavable Moiety is

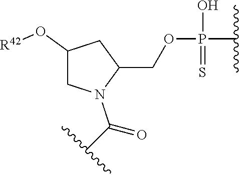

wherein the Therapeutic Oligonucleotide is attached to the —P—.

In certain embodiments Cleavable Moiety is

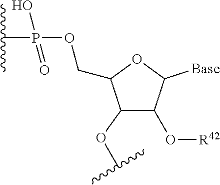

wherein the Therapeutic Oligonucleotide is attached to the —P—.

In certain embodiments Cleavable Moiety is

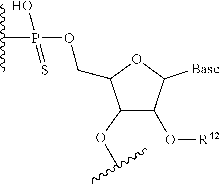

wherein the Therapeutic Oligonucleotide is attached to the —P—.

In certain embodiments Cleavable Moiety is

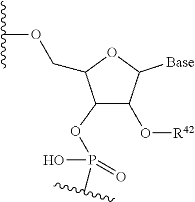

wherein the Therapeutic Oligonucleotide is attached to the —P—.

In certain embodiments Cleavable Moiety is

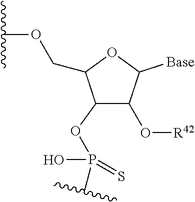

wherein the Therapeutic Oligonucleotide is attached to the —P—.

In certain embodiments Cleavable Moiety is

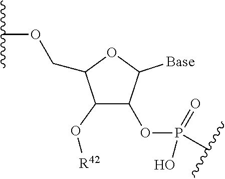

wherein the Therapeutic Oligonucleotide is attached to the —P—.

In certain embodiments Cleavable Moiety is

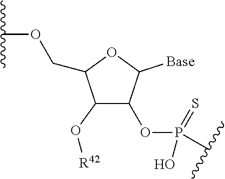

wherein the Therapeutic Oligonucleotide is attached to the —P—.

In certain embodiments R⁴² is hydrogen.

In certain embodiments R⁴² is alkyl.

In certain embodiments R⁴² is alkyl-O-alkyl.

In certain embodiments R⁴² is or C(O)R⁴³.

In certain embodiments R⁴³ is hydrogen.

In certain embodiments R⁴³ is alkyl.

In certain embodiments R⁴³ is haloalkyl.

In certain embodiments the Cleavable Moiety comprises

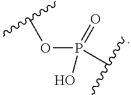 [SMILES: *OP(*)(=O)O]

In certain embodiments the Cleavable Moiety comprises

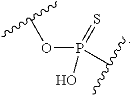 [SMILES: *OP(*)(O)=S]

In certain embodiments the Cleavable Moiety comprises

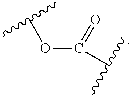 [SMILES: *OC(*)=O]

In certain embodiments the Cleavable Moiety comprises

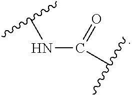 [SMILES: *NC(*)=O]

In certain embodiments the Cleavable Moiety comprises

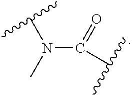 [SMILES: *C(=O)N(*)C]

In certain embodiments Base is

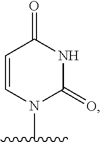 [SMILES: *n1ccc(=O)[nH]c1=O]

In certain embodiments Base is

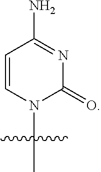 [SMILES: *n1ccc(N)nc1=O]

In certain embodiments Base is

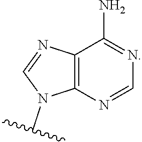 [SMILES: *n1cnc2c(N)ncnc21]

In certain embodiments Base is

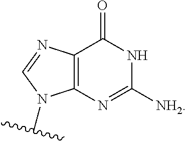 [SMILES: *n1cnc2c(=O)[nH]c(N)nc21]

In certain embodiments Base is

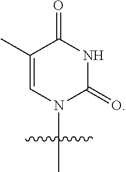 [SMILES: *n1cc(C)c(=O)[nH]c1=O]

In certain embodiments Base is

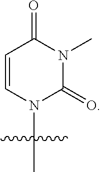 [SMILES: *n1ccc(=O)n(C)c1=O]

In certain embodiments Base is

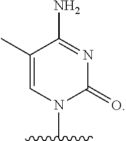 [SMILES: *n1cc(C)c(N)nc1=O]

In certain embodiments Base is

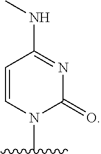 [SMILES: *n1ccc(NC)nc1=O]

In certain embodiments Base is

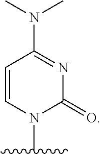 [SMILES: *n1ccc(N(C)C)nc1=O]

In certain embodiments Base is

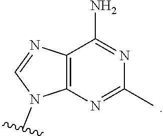 [SMILES: *n1cnc2c(N)nc(C)nc21]

In certain embodiments Base is

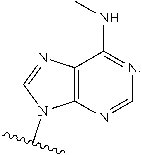 [SMILES: *n1cnc2c(NC)ncnc21]

In certain embodiments Base is

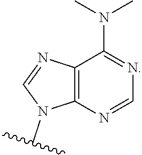 [SMILES: *n1cnc2c(N(C)C)ncnc21]

In certain embodiments Base is

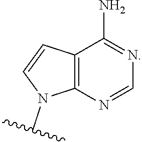 [SMILES: *n1ccc2c(N)ncnc21]

In certain embodiments Base is

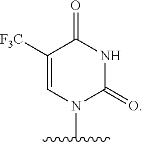 [SMILES: *n1cc(C(F)(F)F)c(=O)[nH]c1=O]

In certain embodiments Base is

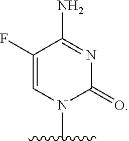 [SMILES: *n1cc(F)c(N)nc1=O]

In certain embodiments Base is

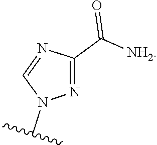 [SMILES: *n1cnc(C(N)=O)n1]

In certain embodiments Base is

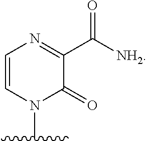 [SMILES: *n1ccnc(C(N)=O)c1=O]

In certain embodiments Base is

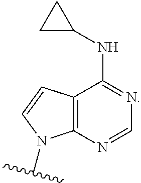 [SMILES: *n1ccc2c(NC3CC3)ncnc21]

In certain embodiments Base is

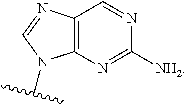 [SMILES: *n1cnc2cnc(N)nc21]

In certain embodiments Base is

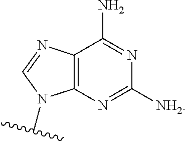 [SMILES: *n1cnc2c(N)nc(N)nc21]

In certain embodiments Base is

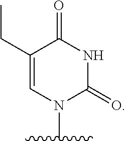 [SMILES: *n1cc(CC)c(=O)[nH]c1=O]

In certain embodiments Base is

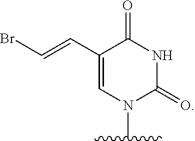 [SMILES: *n1cc(/C=C/Br)c(=O)[nH]c1=O]

In certain embodiments Base is

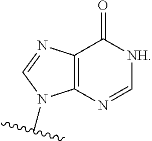 [SMILES: *n1cnc2c(=O)[nH]cnc21]

In certain embodiments Base is

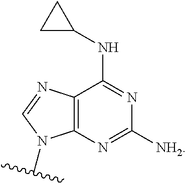 [SMILES: *n1cnc2c(NC3CC3)nc(N)nc21]

In certain embodiments Base is

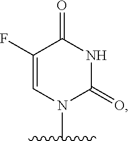 [SMILES: *n1cc(F)c(=O)[nH]c1=O]

In certain embodiments Base is

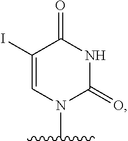 [SMILES: *n1cc(I)c(=O)[nH]c1=O]

In certain embodiments Base is

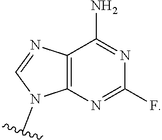 [SMILES: *n1cnc2c(N)nc(F)nc21]

In certain embodiments Cleavable Moiety is

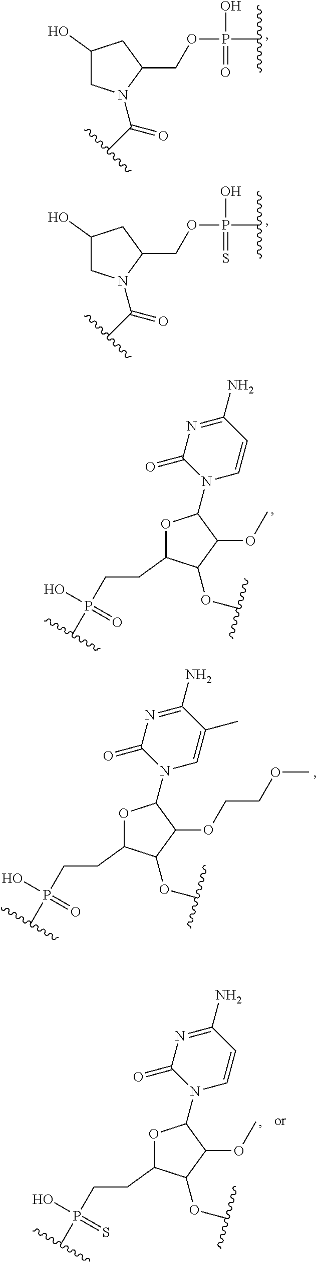

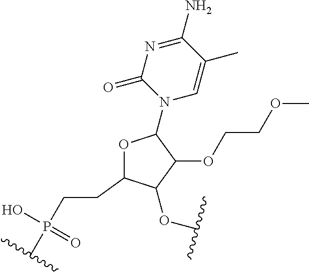 [SMILES: *OC1C(CCP(*)(=O)O)OC(n2cc(C)c(N)nc2=O)C1OCCOC]

wherein the Therapeutic Oligonucleotide is attached to the —P—.

In other embodiments Cleavable Moiety is

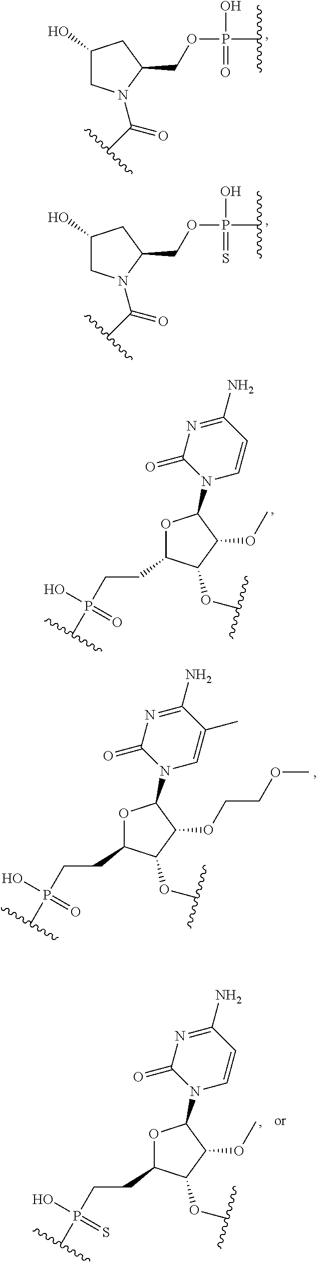

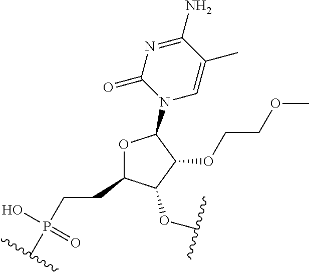 [SMILES: *O[C@H]1[C@@H](OCCOC)[C@H](n2cc(C)c(N)nc2=O)O[C@@H]1CCP(*)(=O)O]

wherein the Therapeutic Oligonucleotide is attached to the —P—.

In certain embodiments Cleavable Moiety is

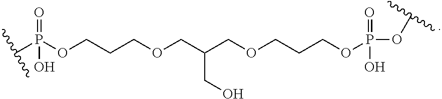 [SMILES: *OP(=O)(O)OCCCOCC(CO)COCCCOP(*)(=O)O ; "."]

In certain embodiments Cleavable Moiety is

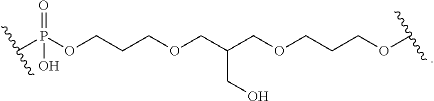 [SMILES: *OCCCOCC(CO)COCCCOP(*)(=O)O ; "."]

In certain embodiments Cleavable Moiety is

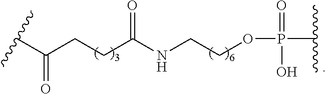

In certain embodiments Cleavable Moiety is

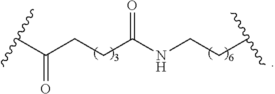

In certain embodiments Cleavable Moiety is

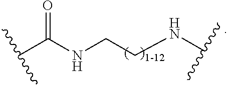

In certain embodiments Cleavable Moiety is

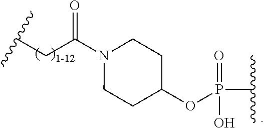

In certain embodiments Cleavable Moiety is

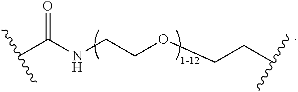

In certain embodiments, a Cleavable Moiety is a cleavable bond. In certain embodiments, a Cleavable Moiety comprises a cleavable bond. In certain embodiments, the Cleavable Moiety comprises a phosphate or phosphodiester. In certain embodiments, the Cleavable Moiety is a cleavable nucleoside or nucleoside analog. In certain embodiments, the nucleoside or nucleoside analog comprises an optionally protected heterocyclic base selected from a purine, substituted purine, pyrimidine or substituted pyrimidine. In certain embodiments, the Cleavable Moiety is a nucleoside comprising an optionally protected heterocyclic base selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. In certain embodiments, the Cleavable Moiety is 2′-deoxy nucleoside that is attached to the 3′ position of the Therapeutic Oligonucleotide by a phosphodiester linkage and is attached to the linker by a phosphodiester or phosphorothioate linkage. In certain embodiments, the Cleavable Moiety is 2′-deoxy adenosine that is attached to the 3′ position of the Therapeutic Oligonucleotide by a phosphodiester linkage and is attached to the linker by a phosphodiester or phosphorothioate linkage. In certain embodiments, the Cleavable Moiety is 2′-deoxy adenosine that is attached to the 3′ position of the Therapeutic Oligonucleotide by a phosphodiester linkage and is attached to the linker by a phosphodiester linkage.

In certain embodiments the Cleavable Moiety is attached to the 3′ position of the Therapeutic Oligonucleotide.

In certain embodiments the Cleavable Moiety is attached to the 5′ position of the Therapeutic Oligonucleotide.

In certain embodiments the Cleavable Moiety is attached to the 2′ position of the Therapeutic Oligonucleotide on the 3′ end of the sequence. In certain embodiments the Cleavable Moiety is attached to the 2′ position of the Therapeutic Oligonucleotide on the 5′ end of the sequence.

In certain embodiments, the Cleavable Moiety is attached to the Therapeutic Oligonucleotide by a phosphodiester linkage or a phosphorothioate linkage.

In certain embodiments, the Cleavable Moiety is cleaved after the complex has been administered to an animal only after being internalized by a targeted cell. Inside the cell the Cleavable Moiety is cleaved thereby releasing the active Therapeutic Oligonucleotide. In certain aspects the Cleavable Moiety is cleaved by one or more nucleases within the cell. In certain embodiments, the one or more nucleases cleave the phosphodiester linkage between the Cleavable Moiety and the linker.

In certain embodiments, the Cleavable Moiety has a structure selected from among the following:

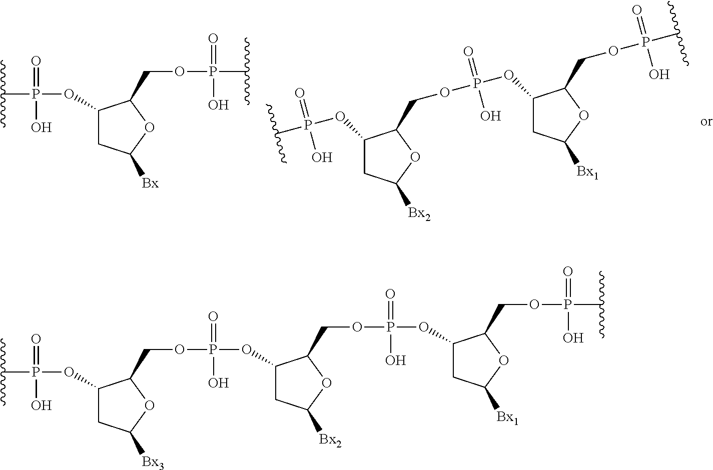

wherein each of Bx, Bx₁, Bx₂, and Bx₃ is independently a Base.

In certain embodiments, the Cleavable Moiety has the following structure:

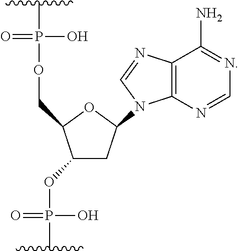 [SMILES: *P(=O)(O)OC[C@H]1O[C@@H](n2cnc3c(N)ncnc32)C[C@@H]1OP(*)(=O)O]

### EXAMPLES

### List of Abbreviations

| Abbreviation | Full name |
|---|---|
| CPG | Controlled pore glass |
| SS | Sense strand |
| AS | Antisene strand |
| ADTT | Xanthane hydride |
| IP | Ion-pairing |
| RP | Reverse-phase |
| PFP | Pentafluorophenol |
| OD | Optical density |
| moeX | 2'-O-methoxyethyl (MOE) protected nucleotide X; |
| | wherein X can be a canonical (A, C, G, T) or non- |
| | canonical nucleotide. For example, “moeG” is |
| | 2'-O-methoxyethyl guanosine. |
| dX | deoxy (DNA) nucleotide X; wherein X can |
| | be a canonical (A ,C, G, T) or non- |
| | canonical nucleotide |

### Example 1. Synthesis of Intermediates

### Preparation of 1-((2R,3R,4R,5S)-3,4-diacetoxy-5-((6-(trifluoromethyl)pyrazin-2-yl)amino)tetrahydro-2H-pyran-2-yl)-15,15-bis((3-((6-(((2R,3R,4R,5S)-3,4-diacetoxy-5-((6-(trifluoromethyl)pyrazin-2-yl)amino)tetrahydro-2H-pyran-2-yl)methoxy)hexyl)amino)-3-oxopropoxy)methyl)-10,17-dioxo-2,13-dioxa-9,16-diazahenicosan-21-oic acid (Intermediate 1)

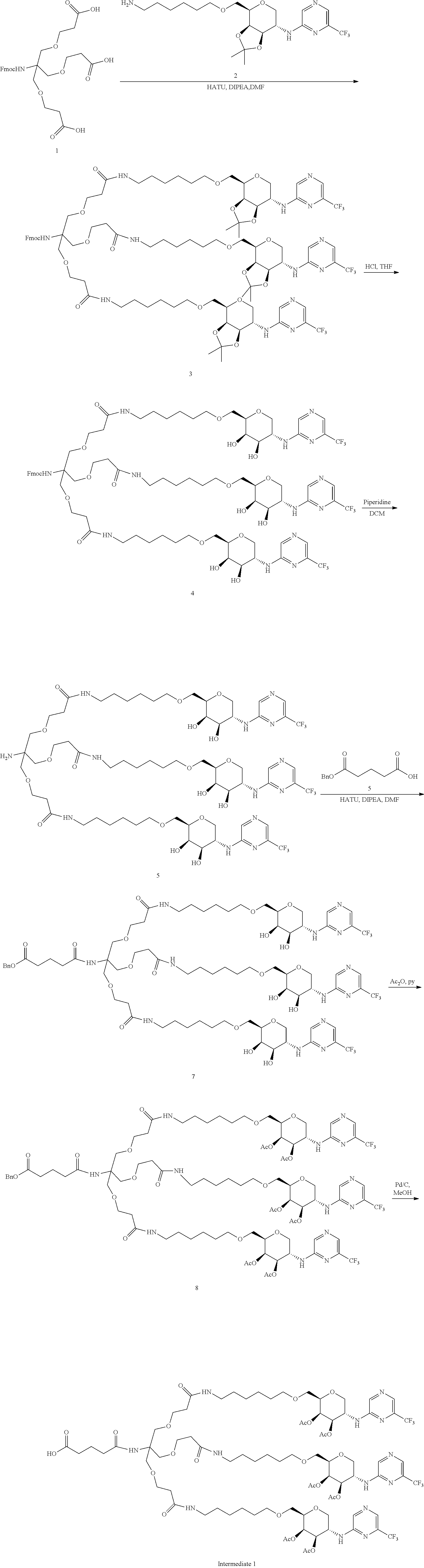

Step 1: To a solution of 1 (1.00 eq) in DMF was added HATU (4.50 eq) and DIEA (6.00 eq), the mixture was stirred at 25° C. for 0.5 hour, then 2 (5.00 eq) was added, the resulting mixture was stirred at 25° C. for 10 hours. The mixture was concentrated under reduced pressure. The residue was purified by reverse phase (C18, 5-80% MeCN in water, 0.05% TFA) to give 3 as colorless oil. Yield: 33.25%. LCMS found: [M+H]⁺=1851.

Step 2: To a solution of 3 (1.00 eq) in THF (2 mL) was added HCl (0.5 mL, 2 N in H₂O) at 0° C., the mixture was stirred at 25° C. for 10 hours under nitrogen atmosphere. The mixture was concentrated under reduced pressure. The residue was purified by reverse phase (5-45% CH₃CN in H₂O, 0.05% TFA) to afford 4 as yellow oil. Yield: 76.39%. LCMS found: [M+H]⁺=1731.

Step 3: To a solution of 4 (1.00 eq) in DCM was added Piperidine (0.1 mL), the mixture was stirred at 25° C. for 10 hours. The mixture was concentrated under reduced pressure. The residue was purified by reverse phase (C18, 5-40% MeCN in water, 0.05% TFA) to afford 5 as colorless oil. Yield: 68.88%. LCMS found: [M+H]⁺=1509.

Step 4: To a solution of 6 (1.00 eq) in DMF was added HATU (1.50 eq) and DIEA (2.00 eq), the mixture was stirred at 25° C. for 0.5 hour, then 7 (1.10 eq) was added, the resulting mixture was stirred at 25° C. for 10 hours. The mixture was concentrated under reduced pressure. The residue was purified by reverse phase (C18, 5-60% MeCN in water, 0.05% TFA) to give 8 as colorless oil. Yield: 53.42%. LCMS found: [M+H]⁺=1713.

Step 5: To a solution of 8 (1.00 eq) in Py (3 mL) was added Ac₂O (1 mL) at 0° C., the mixture was stirred at 25° C. for 10 hours under nitrogen atmosphere. The mixture was concentrated under reduced pressure. The residue was purified by reverse phase (C18, 5-88% MeCN in water, 0.05% TFA) to afford 9 as colorless oil. Yield: 58.11%. LCMS found: [M+H]⁺=1965.

Step 6: To a solution of 9 (1.00 eq) in MeOH was added Pd/C (10% purity, 0.20 eq), the mixture was degassed and purged with hydrogen for several times, then stirred at 25° C. for 2 hours. LCMS showed the staring material was consumed and the desired mass was observed. The mixture was filtered, the filtrate was concentrated under reduced pressure. The residue was purified by reverse phase (5-45% CH₃CN in H₂O) to afford Intermediate 1 as white solid. Yield: 21.22%. LCMS found: [M+H]⁺=1875. ¹H NMR (400 MHz, MeOD): δ 8.18-7.96 (m, 6H), 5.49 (d, J=3.0 Hz, 3H), 5.07 (dd, J=11.1, 3.2 Hz, 3H), 4.59-4.45 (m, 3H), 4.17 (dd, J=11.2, 5.2 Hz, 3H), 3.85 (t, J=6.4 Hz, 3H), 3.73-3.62 (m, 12H), 3.55-3.35 (m, 15H), 3.18 (t, J=7.1 Hz, 6H), 2.41 (t, J=6.0 Hz, 6H), 2.34 (t, J=7.5 Hz, 2H), 2.24 (t, J=7.3 Hz, 2H), 2.15 (s, 9H), 1.89 (s, 9H), 1.62-1.45 (m, 14H), 1.42-1.30 (m, 12H).

### Preparation of 5-(((2R,3R,4R,5S)-3,4-diacetoxy-5-((6-(trifluoromethyl)pyrazin-2-yl)amino)tetrahydro-2H-pyran-2-yl)methoxy)pentanoic acid (Intermediate 2)

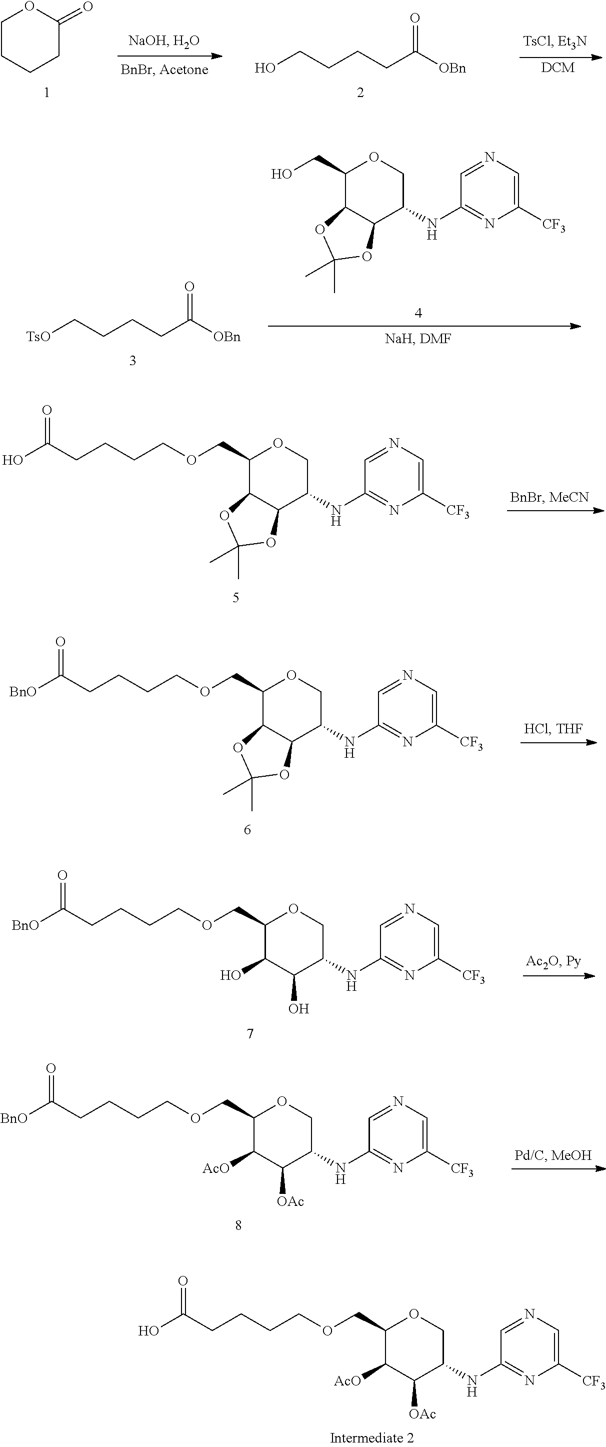

Step 1: A solution of 1 (1.00 eq) and NaOH (1.00 eq) in water (100 mL) was stirred overnight at 70° C. The reaction mixture was cooled to rt and concentrated in vacuo to give white solid. This solid was suspended in acetone (100 mL) and refluxed overnight with BnBr (1.20 eq) and TBAB (0.005 eq). Acetone was removed in vacuo to afford an oily residue, which was dissolved in EtOAc and washed with sat. NaHCO₃(aq.) and brine. The organic layer was dried over Na₂SO₄ and concentrated in vacuo. The residue was purified by silica gel chromatography (PE/EA=10/1 to 1/1) to afford 2 as a colorless oil. Yield: 82.1%. LC-MS (ESI) found: 209 [M+H]⁺.

Step 2: To a solution of 2 (1.00 eq) in DCM (2 mL) was added TsCl (1.50 eq) and Et₃N (2.00 eq) at 0° C., the mixture was stirred at 25° C. for 10 hours under nitrogen atmosphere. The mixture was concentrated under reduced pressure. The residue was purified by silica gel chromatography (PE/EA=10/1 to 3/1) to afford 3 as a colorless oil. Yield: 50.3%. LCMS found: [M+H]⁺=363.

Step 3: To a solution of 4 (1.00 eq) in DMF was added NaH (3.00 eq) at 0° C., the mixture was stirred at 25° C. for 0.5 hour, then 3 (1.20 eq) was added, the resulting mixture was stirred at 60° C. for 3 hours under nitrogen atmosphere. The mixture was quenched with saturated solution of NH₄Cl, then concentrated under reduced pressure. The residue was purified by reverse phase (5-80% CH₃CN in H₂O, 0.1% TFA) to afford 5 as a yellow oil. Yield: 34.93%. LCMS found: [M+H]⁺=450.

Step 4: To a solution of 5 (1.00 eq) in MeCN was added K₂CO₃ (1.10 eq) and BnBr (1.00 eq), the mixture was stirred at 70° C. for 5 hours. The mixture was concentrated under reduced pressure. The residue was purified by reverse phase (C18, 5-90% MeCN in water, 0.05% TFA) to give 6 as yellow oil. Yield: 74.97%. LCMS found: [M+H]⁺=540.

Step 5: To a solution of 6 (1.00 eq) in THF (2 mL) was added HCl (0.5 mL, 2 N in H₂O) at 0° C., the mixture was stirred at 25° C. for 10 hours under nitrogen atmosphere. The mixture was concentrated under reduced pressure. The residue was purified by reverse phase (5-45% CH₃CN in H₂O, 0.05% TFA) to afford 7 as colorless oil. Yield: 73.91%. LCMS found: [M+H]⁺=500.

Step 6: To a solution of 7 (1.00 eq) in Py (3 mL) was added Ac₂O (1 mL) at 0° C., the mixture was stirred at 25° C. for 10 hours under nitrogen atmosphere. The mixture was concentrated under reduced pressure. The residue was purified by reverse phase (C18, 5-88% MeCN in water, 0.05% TFA) to afford 8 as colorless oil. Yield: 72.43%. LCMS found: [M+H]⁺=584.

Step 7: To a solution of 8 (1.00 eq) in MeOH was added Pd/C (10% purity, 0.20 eq), the mixture was degassed and purged with hydrogen for several times, then stirred at 25° C. for 2 hours. LCMS showed the staring material was consumed and the desired mass was observed. The mixture was filtered, the filtrate was concentrated under reduced pressure. The residue was purified by reverse phase (C18, 5-45% CH₃CN in H₂O) to afford Intermediate 2 as white solid. Yield: 74.18%. LCMS found: [M+H]⁺=494. ¹H NMR (400 MHz, MeOD): δ 8.06 (d, J=6.0 Hz, 2H), 5.48 (d, J=2.6 Hz, 1H), 5.07 (dd, J=11.1, 3.3 Hz, 1H), 4.51 (td, J=10.9, 5.1 Hz, 1H), 4.17 (dd, J=11.2, 5.2 Hz, 1H), 3.85 (t, J=6.0 Hz, 1H), 3.57-3.46 (m, 2H), 3.45-3.38 (m, 2H), 3.38-3.32 (m, 1H), 2.30 (t, J=7.1 Hz, 2H), 2.15 (s, 3H), 1.89 (s, 3H), 1.70-1.51 (m, 4H).

### Preparation of 1-((2R,3R,4R,5S)-3,4-diacetoxy-5-((6-(trifluoromethyl)pyrazin-2-yl)amino)tetrahydro-2H-pyran-2-yl)-10,17-dioxo-2,13-dioxa-9,16-diazahenicosan-21-oic acid (Intermediate 3)

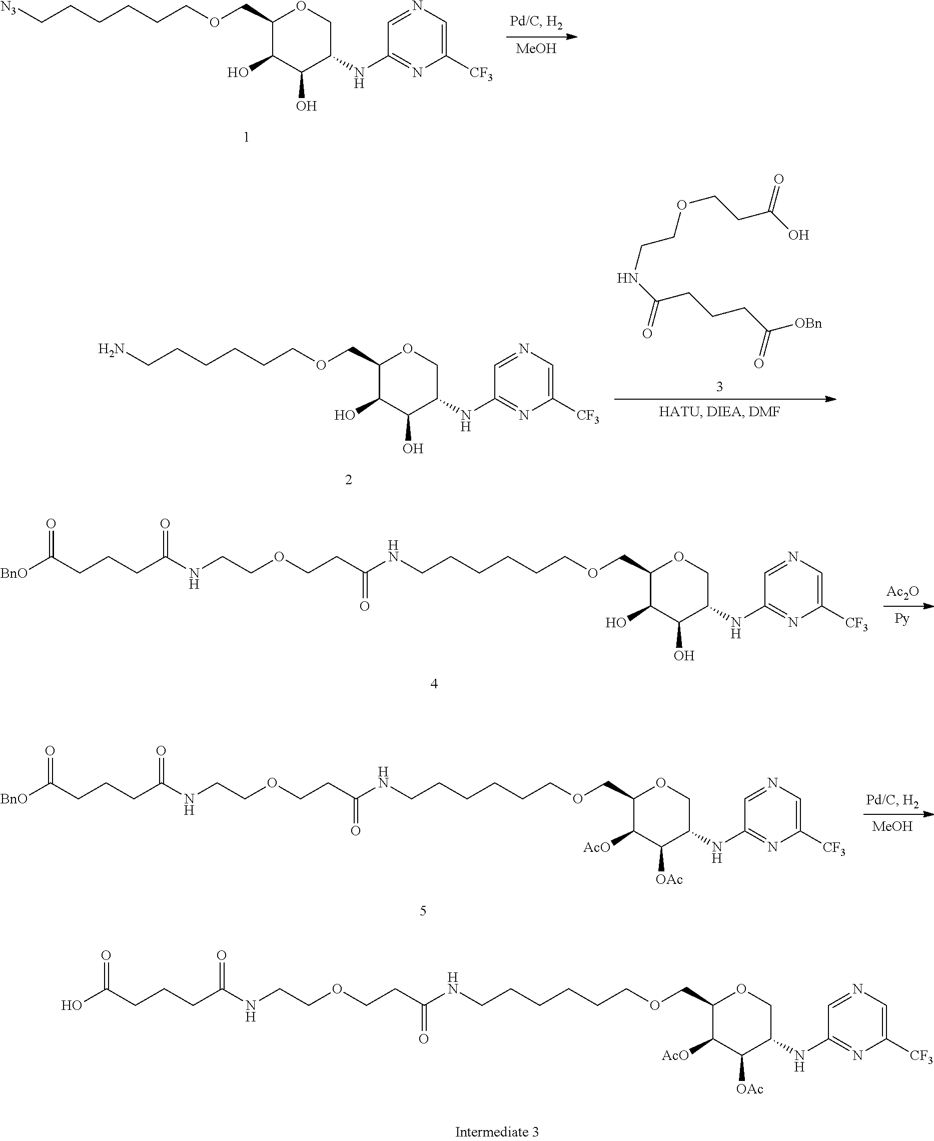

Step 1: To a solution of 1 (1.00 eq) in MeOH was added Pd/C (10% purity, 0.20 eq), the mixture was degassed and purged with hydrogen for several times, then stirred at 25° C. for 1 hour under hydrogen atmosphere (15 psi). LCMS showed the staring material was consumed and the desired mass was observed. The mixture was filtered, the filtrate was concentrated under reduced pressure to afford 2 as a yellow oil. Yield: 98.2%. LC-MS (ESI) found: 409 [M+H]⁺.

Step 2: To a solution of 3 (1.20 eq) in DMF was added HATU (1.30 eq) and DIEA (4.00 eq), the mixture was stirred at 25° C. for 0.5 hour, then 2 (1.00 eq) was added, the resulting mixture was stirred at 25° C. for 2 hours. The mixture was diluted with water, extracted with EtOAc, the organic layer was washed with brine, dried over anhydrous sodium sulfate, filtered and concentrated under reduced pressure. The residue was purified by silica gel chromatography (DCM/MeOH=100/1 to 10/1) to afford 4 as a yellow oil. Yield: 44.0%. LC-MS (ESI) found: 728 [M+H]⁺.

Step 3: To a solution of 4 (1.00 eq) in Py was added Ac₂O (20.00 eq), the mixture was stirred at 30° C. for 3 hours. The mixture was diluted with water, extracted with EtOAc, the organic layer was washed with brine, dried over anhydrous sodium sulfate, filtered and concentrated under reduced pressure. The residue was purified by silica gel chromatography (DCM/MeOH=100/1 to 10/1) to afford 5 as a yellow oil. Yield: 87.3%. LC-MS (ESI) found: 812 [M+H]⁺.

Step 4: To a solution of 5 (1.00 eq) in MeOH was added Pd/C (10% purity, 0.20 eq), the mixture was degassed and purged with hydrogen for several times, then stirred at 25° C. for 1 hour under hydrogen atmosphere (15 psi). LCMS showed the staring material was consumed and the desired mass was observed. The mixture was filtered, the filtrate was concentrated under reduced pressure. The residue was purified by reverse phase (5-40% CH₃CN in H₂O) to afford Intermediate 3 as a white solid. Yield: 42.0%. LC-MS (ESI) found: 722 [M+H]⁺. ¹H NMR (400 MHz, CD₃OD): δ 8.08 (d, J=7.1 Hz, 2H), 5.51 (d, J=2.6 Hz, 1H), 5.09 (dd, J=11.1, 2.3 Hz, 1H), 4.57-4.50 (m, 1H), 4.19 (dd, J=11.1, 5.1 Hz, 1H), 3.87 (t, J=6.3 Hz, 1H), 3.71 (t, J=6.0 Hz, 2H), 3.57-3.41 (m, 6H), 3.40-3.35 (m, 3H), 3.20 (t, J=6.9 Hz, 2H), 2.45 (t, J=5.9 Hz, 2H), 2.34 (t, J=7.3 Hz, 2H), 2.27 (t, J=7.4 Hz, 2H), 2.17 (s, 3H), 1.95-1.87 (m, 5H), 1.59-1.48 (m, 4H), 1.37 (d, J=3.2 Hz, 4H). ¹⁹F NMR (376 MHz, CD₃OD): δ −70.35 (s).

### Preparation of 5-(((1S,2R,3R,4R,5S)-2,3-diacetoxy-4-((6-(trifluoromethyl)pyridin-2-yl)amino)-6,8-dioxabicyclo[3.2.1]octan-1-yl)methoxy)pentanoic acid (Intermediate 6)

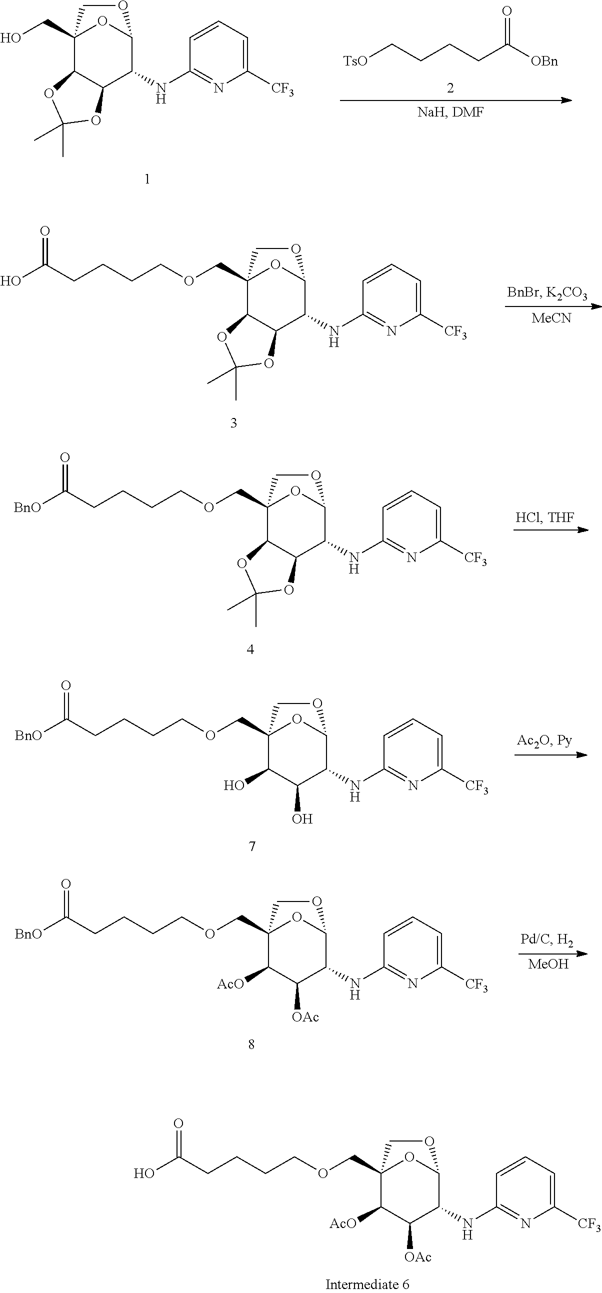

Step 1: To a solution of ((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methanol (1.00 eq) in DMF is added NaH (60% purity, 3.00 eq) at 0° C., the mixture is stirred at 25° C. for 0.5 hour, then benzyl 5-(tosyloxy)pentanoate (1.20 eq) is added, the resulting mixture is stirred at 60° C. for 3 hours under nitrogen atmosphere. The mixture is quenched with saturated solution of NH₄Cl, then is concentrated under reduced pressure. The residue is purified by reverse phase to afford 5-(((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methoxy)pentanoic acid.

Step 2: To a solution of 5-(((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methoxy)pentanoic acid (1.00 eq) in MeCN is added K₂CO₃ (1.10 eq) and BnBr (1.00 eq), the mixture is stirred at 80° C. for 5 hours. The mixture is concentrated under reduced pressure. The residue is purified by silica gel chromatography to give benzyl 5-(((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methoxy)pentanoate.

Step 3: To a solution of benzyl 5-(((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methoxy)pentanoate (1.00 eq) in THF is added HCl (2M) at 0° C., the mixture is stirred at 25° C. for 10 hours under nitrogen atmosphere. The mixture is concentrated under reduced pressure. The residue is purified by reverse phase to afford benzyl 5-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)pentanoate.

Step 4: To a solution of benzyl 5-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)pentanoate (1.00 eq) in pyridine is added Ac₂O (3.00 eq) at 0° C., the mixture is stirred at 25° C. for 10 hours under nitrogen atmosphere. The mixture is concentrated under reduced pressure. The residue is purified by reverse phase to afford (1S,2R,3R,4R,5S)-1-(((5-(benzyloxy)-5-oxopentyl)oxy)methyl)-4-((6-(trifluoromethyl)pyridin-2-yl)amino)-6,8-dioxabicyclo[3.2.1]octane-2,3-diyl diacetate.

Step 5: To a solution of (1S,2R,3R,4R,5S)-1-(((5-(benzyloxy)-5-oxopentyl)oxy)methyl)-4-((6-(trifluoromethyl)pyridin-2-yl)amino)-6,8-dioxabicyclo[3.2.1]octane-2,3-diyl diacetate (1.00 eq) in MeOH is added Pd/C (10% purity, 0.20 eq), the mixture is degassed and purged with hydrogen for several times, then is stirred at 25° C. for 2 hours under hydrogen atmosphere (15 psi). The mixture is filtered, the filtrate is concentrated under reduced pressure. The residue is purified by reverse phase to afford 5-(((1S,2R,3R,4R,5S)-2,3-diacetoxy-4-((6-(trifluoromethyl)pyridin-2-yl)amino)-6,8-dioxabicyclo[3.2.1]octan-1-yl)methoxy)pentanoic acid (Intermediate 6).

### Preparation of 1-((1S,2R,3R,4R,5S)-2,3-diacetoxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oic acid (Intermediate 7)

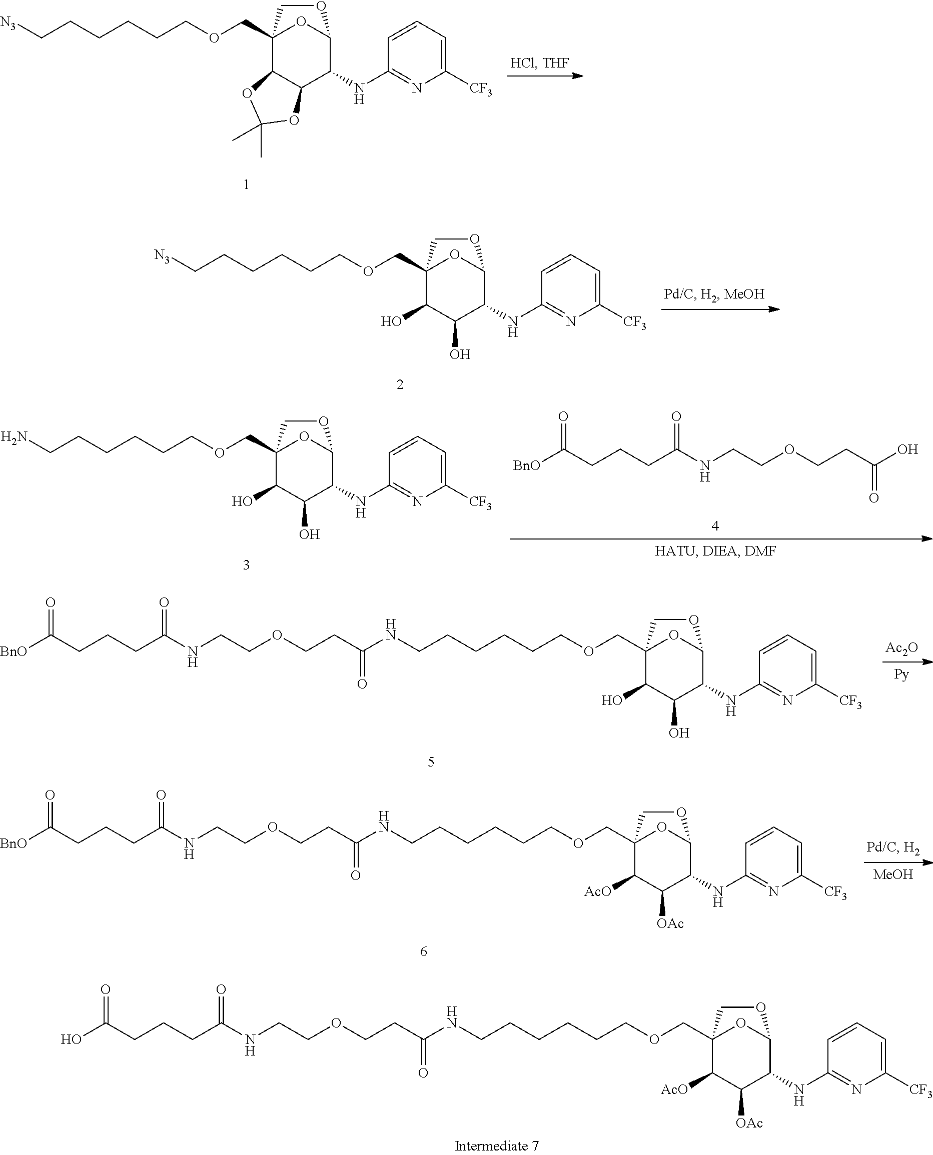

Step 1: To a solution of N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyridin-2-amine (1.00 eq) in THF is added HCl (2M) at 0° C., the mixture is stirred at 25° C. for 12 hours under nitrogen atmosphere. The mixture is concentrated under reduced pressure. The residue is purified by reverse phase to afford (1S,2R,3R,4R,5S)-1-(((6-azidohexyl)oxy)methyl)-4-((6-(trifluoromethyl)pyridin-2-yl)amino)-6,8-dioxabicyclo[3.2.1]octane-2,3-diol.

Step 2: To a solution of (1S,2R,3R,4R,5S)-1-(((6-azidohexyl)oxy)methyl)-4-((6-(trifluoromethyl)pyridin-2-yl)amino)-6,8-dioxabicyclo[3.2.1]octane-2,3-diol (1.00 eq) in MeOH is added Pd/C (10% purity, 0.30 eq), the mixture is degassed and purged with hydrogen for several times, then is stirred at 25° C. for 4 hours under hydrogen atmosphere (15 psi). The mixture is filtered, the filtrate is concentrated under reduced pressure. The residue is purified by reverse phase to afford (1S,2R,3R,4R,5S)-1-(((6-aminohexyl)oxy)methyl)-4-((6-(trifluoromethyl)pyridin-2-yl)amino)-6,8-dioxabicyclo[3.2.1]octane-2,3-diol.

Step 3: To a solution of 3-(2-(5-(benzyloxy)-5-oxopentanamido)ethoxy)propanoic acid (1.20 eq) in DMF is added HATU (1.30 eq) and DIEA (4.00 eq), the mixture is stirred at 25° C. for 0.5 hour, then (1S,2R,3R,4R,5S)-1-(((6-aminohexyl)oxy)methyl)-4-((6-(trifluoromethyl)pyridin-2-yl)amino)-6,8-dioxabicyclo[3.2.1]octane-2,3-diol (1.00 eq) is added, the resulting mixture is stirred at 25° C. for 6 hours. The mixture is diluted with water, extracted with EtOAc, the organic layer is washed with brine, dried over anhydrous sodium sulfate, filtered and concentrated under reduced pressure. The residue is purified by silica gel chromatography to afford benzyl 1-((1S,2R,3R,4R,5S)-2,3-dihydroxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oate.

Step 4: To a solution of benzyl 1-((1S,2R,3R,4R,5S)-2,3-dihydroxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oate (1.00 eq) in pyridine is added Ac₂O (10.00 eq), the mixture is stirred at 25° C. for 8 hours. The mixture is diluted with water, extracted with EtOAc, the organic layer is washed with brine, dried over anhydrous sodium sulfate, filtered and concentrated under reduced pressure. The residue is purified by silica gel chromatography to afford (1S,2R,3R,4R,5S)-4-((6-(trifluoromethyl)pyridin-2-yl)amino)-1-(3,7,14-trioxo-1-phenyl-2,11,22-trioxa-8,15-diazatricosan-23-yl)-6,8-dioxabicyclo[3.2.1]octane-2,3-diyl diacetate.

Step 5: To a solution of (1S,2R,3R,4R,5S)-4-((6-(trifluoromethyl)pyridin-2-yl)amino)-1-(3,7,14-trioxo-1-phenyl-2,11,22-trioxa-8,15-diazatricosan-23-yl)-6,8-dioxabicyclo[3.2.1]octane-2,3-diyl diacetate (1.00 eq) in MeOH is added Pd/C (10% purity, 0.20 eq), the mixture is degassed and purged with hydrogen for several times, then stirred at 25° C. for 3 hours under hydrogen atmosphere (15 psi). The mixture is filtered, the filtered cake is washed with MeOH for 3 times, the combined filtrate is concentrated under reduced pressure. The residue is purified by reverse phase to afford 1-((1S,2R,3R,4R,5S)-2,3-diacetoxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oic acid (Intermediate 7).

### Preparation of 15,15-bis((3-((6-(((1S,2R,3R,4R,5S)-2,3-diacetoxy-4-((6-(trifluoromethyl)pyrazin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oic acid (Intermediate 8)

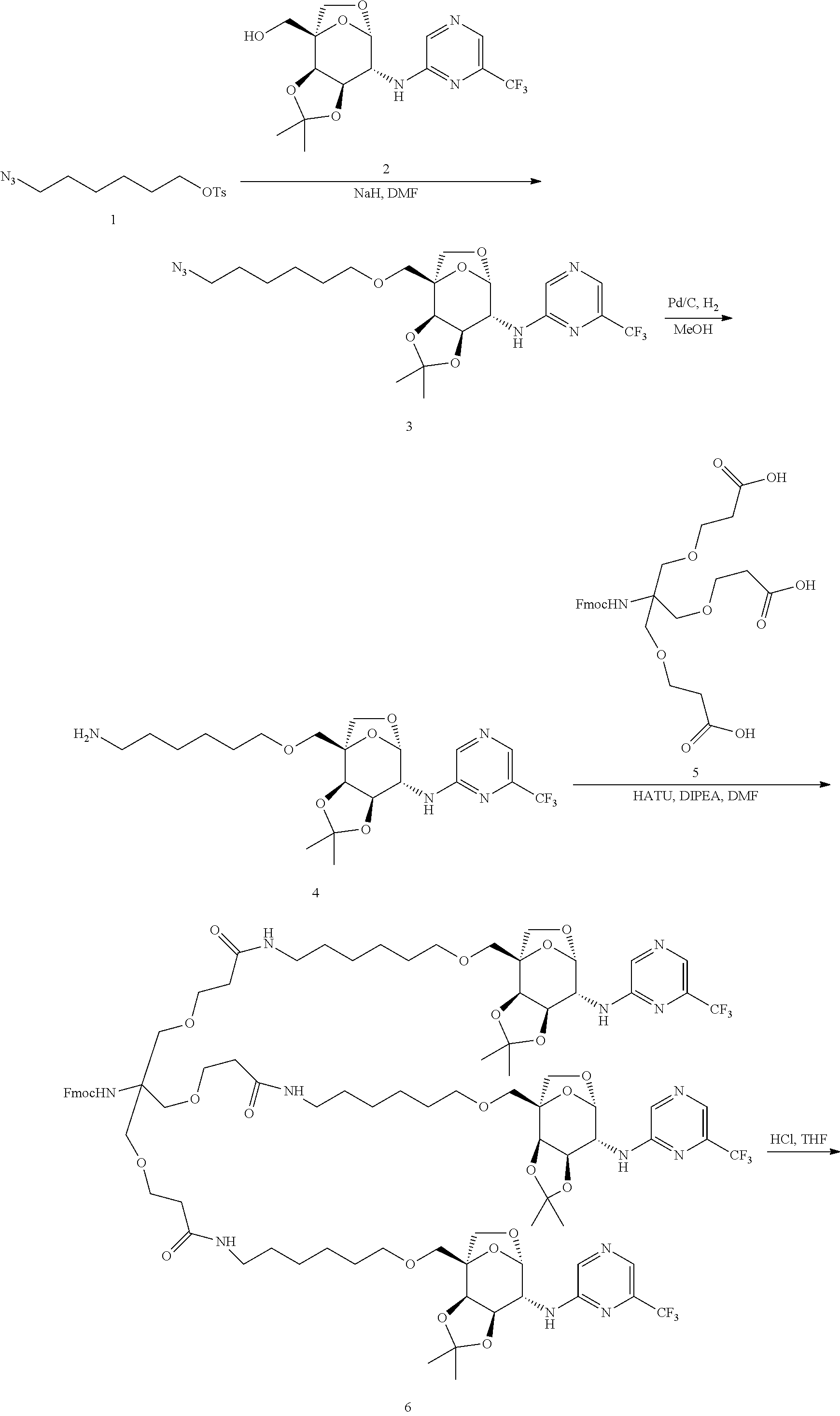

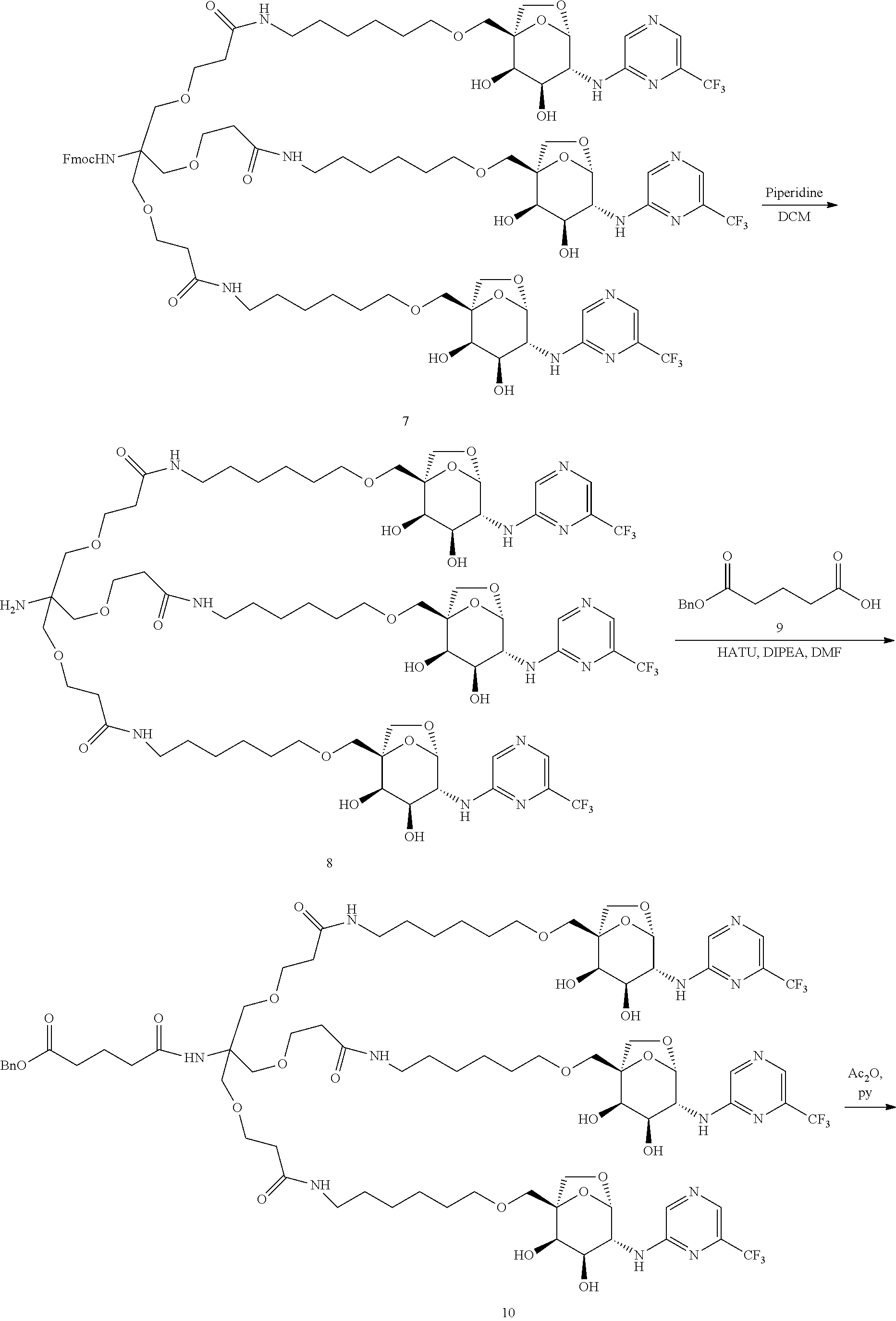

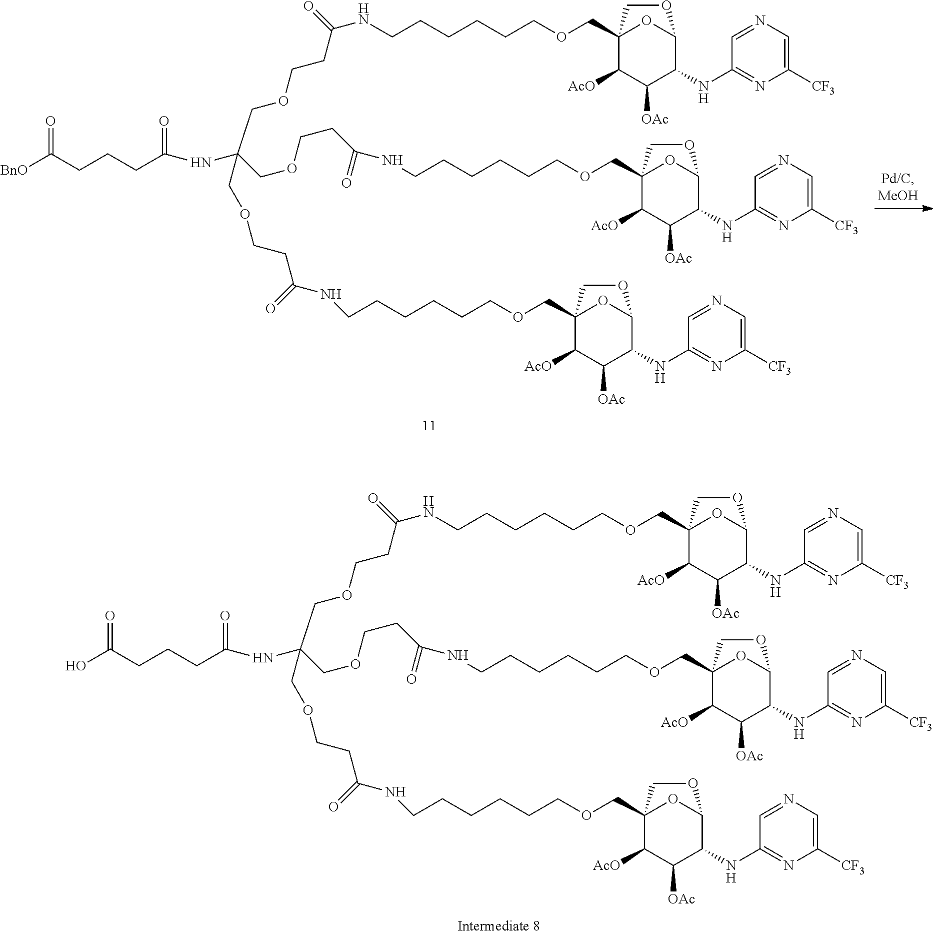

Step 1: To a solution of ((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methanol (1.00 eq) in DMF is added NaH (60% purity, 3.00 eq) at 0° C., the mixture is stirred at 25° C. for 0.5 hour, then 6-azidohexyl 4-methylbenzenesulfonate (1.00 eq) is added, the resulting mixture is stirred at 25° C. for 12 hours under nitrogen atmosphere. The mixture is quenched with saturated solution of NH₄Cl, then extracted with EtOAc, the organic layer is washed with brine, dried over anhydrous sodium sulfate, then concentrated under reduced pressure. The residue is purified by silica gel chromatography to afford N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyrazin-2-amine.

Step 2: To a solution of N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyrazin-2-amine (1.00 eq) in MeOH is added Pd/C (10% purity, 0.20 eq) and NH₃·H₂O (0.1 mL), the mixture is degassed and purged with hydrogen for several times, then is stirred at 25° C. for 1 hour. The mixture is filtered, then the filtrate is concentrated under reduced pressure to afford N-((3aR,4S,7S,8R,8aR)-4-(((6-aminohexyl)oxy)methyl)-2,2-dimethylhexahydro-4,7-epoxy[1,3]dioxolo[4,5-d]oxepin-8-yl)-6-(trifluoromethyl)pyrazin-2-amine.

Step 3: To a solution of 3,3′-((2-((((9H-fluoren-9-yl)methoxy)carbonyl)amino)-2-((2-carboxyethoxy)methyl)propane-1,3-diyl)bis(oxy))dipropionic acid (1.00 eq) in DMF is added HATU (4.50 eq) and DIEA (6.00 eq), the mixture is stirred at 25° C. for 0.5 hour, then N-((3aR,4S,7S,8R,8aR)-4-(((6-aminohexyl)oxy)methyl)-2,2-dimethylhexahydro-4,7-epoxy[1,3]dioxolo[4,5-d]oxepin-8-yl)-6-(trifluoromethyl)pyrazin-2-amine (5.00 eq) is added, the resulting mixture is stirred at 25° C. for 10 hours. The mixture is concentrated under reduced pressure. The residue is purified by reverse phase to afford (9H-fluoren-9-yl)methyl (1,29-bis((3aR,4S,7S,8R,8aR)-2,2-dimethyl-8-((6-(trifluoromethyl)pyrazin-2-yl)amino) tetrahydro-4,7-epoxy[1,3]dioxolo[4,5-d]oxepin-4(5H)-yl)-15-((3-((6-(((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methoxy)hexyl)amino)-3-oxopropoxy)methyl)-10,20-dioxo-2,13,17,28-tetraoxa-9,21-diazanonacosan-15-yl)carbamate.

Step 4: To a solution of (9H-fluoren-9-yl)methyl (1,29-bis((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)-15-((3-((6-(((3aR,4S,7S,8R,8aR)-2,2-dimethyl-8-((6-(trifluoromethyl)pyrazin-2-yl)amino) tetrahydro-4,7-epoxy[1,3]dioxolo[4,5-d]oxepin-4(5H)-yl)methoxy)hexyl)amino)-3-oxopropoxy) methyl)-10,20-dioxo-2,13,17,28-tetraoxa-9,21-diazanonacosan-15-yl)carbamate (1.00 eq) in THF is added HCl (2M) at 0° C., the mixture is stirred at 25° C. for 10 hours under nitrogen atmosphere.

The mixture is concentrated under reduced pressure. The residue is purified by reverse phase to afford (9H-fluoren-9-yl)methyl (15-((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl) amino)-3-oxopropoxy)methyl)-1,29-bis((1S,2R,3R,4R,5S)-2,3-dihydroxy-4-((6-(trifluoromethyl) pyrazin-2-yl)amino)-6,8-dioxabicyclo[3.2.1]octan-1-yl)-10,20-dioxo-2,13,17,28-tetraoxa-9,21-diazanonacosan-15-yl)carbamate.

Step 5: To a solution of (9H-fluoren-9-yl)methyl (15-((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl) amino)-3-oxopropoxy)methyl)-1,29-bis((1S,2R,3R,4R,5S)-2,3-dihydroxy-4-((6-(trifluoromethyl) pyrazin-2-yl)amino)-6,8-dioxabicyclo[3.2.1]octan-1-yl)-10,20-dioxo-2,13,17,28-tetraoxa-9,21-diazanonacosan-15-yl)carbamate (1.00 eq) in DCM is added piperidine (2.00 eq), the mixture is stirred at 25° C. for 10 hours. The mixture is concentrated under reduced pressure. The residue is purified by reverse phase to afford 3,3′-((2-amino-2-((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)amino)-3-oxopropoxy)methyl)propane-1,3-diyl)bis(oxy))bis(N-(6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)propanamide).

Step 6: To a solution of 5-(benzyloxy)-5-oxopentanoic acid (1.00 eq) in DMF is added HATU (1.50 eq) and DIEA (2.00 eq), the mixture is stirred at 25° C. for 0.5 hour, then 3,3′-((2-amino-2-((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)amino)-3-oxopropoxy)methyl)propane-1,3-diyl)bis(oxy))bis(N-(6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)propanamide) (1.10 eq) is added, the resulting mixture is stirred at 25° C. for 10 hours. The mixture is concentrated under reduced pressure. The residue is purified by reverse phase to give benzyl 15,15-bis((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-dihydroxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oate.

Step 7: To a solution of benzyl 15,15-bis((3-((6-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-dihydroxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oate (1.00 eq) in Pyridine is added Ac₂O (10.0 eq) at 0° C., the mixture is stirred at 25° C. for 10 hours under nitrogen atmosphere. The mixture is concentrated under reduced pressure. The residue is purified by reverse phase to afford benzyl 15,15-bis((3-((6-(((1S,2R,3R,4R,5S)-2,3-diacetoxy-4-((6-(trifluoromethyl)pyrazin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-2-oate.

Step 8: To a solution of benzyl 15,15-bis((3-((6-(((1S,2R,3R,4R,5S)-2,3-diacetoxy-4-((6-(trifluoromethyl)pyrazin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-2-oate (1.00 eq) in MeOH is added Pd/C (10% purity, 0.20 eq), the mixture is degassed and purged with hydrogen for several times, then stirred at 25° C. for 2 hours under hydrogen atmosphere (15 psi). The mixture is filtered, the filtrate is concentrated under reduced pressure. The residue is purified by reverse phase to afford 15,15-bis((3-((6-(((1S,2R,3R,4R,5S)-2,3-diacetoxy-4-((6-(trifluoromethyl)pyrazin-2-yl)amino)-6,8-dioxabicyclo [3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oic acid (Intermediate 8).

### Preparation of 5-(((1S,2R,3R,4R,5S)-2,3-diacetoxy-4-((6-(trifluoromethyl)pyrazin-2-yl)amino)-6,8-dioxabicyclo[3.2.1]octan-1-yl)methoxy)pentanoic acid (Intermediate 9)

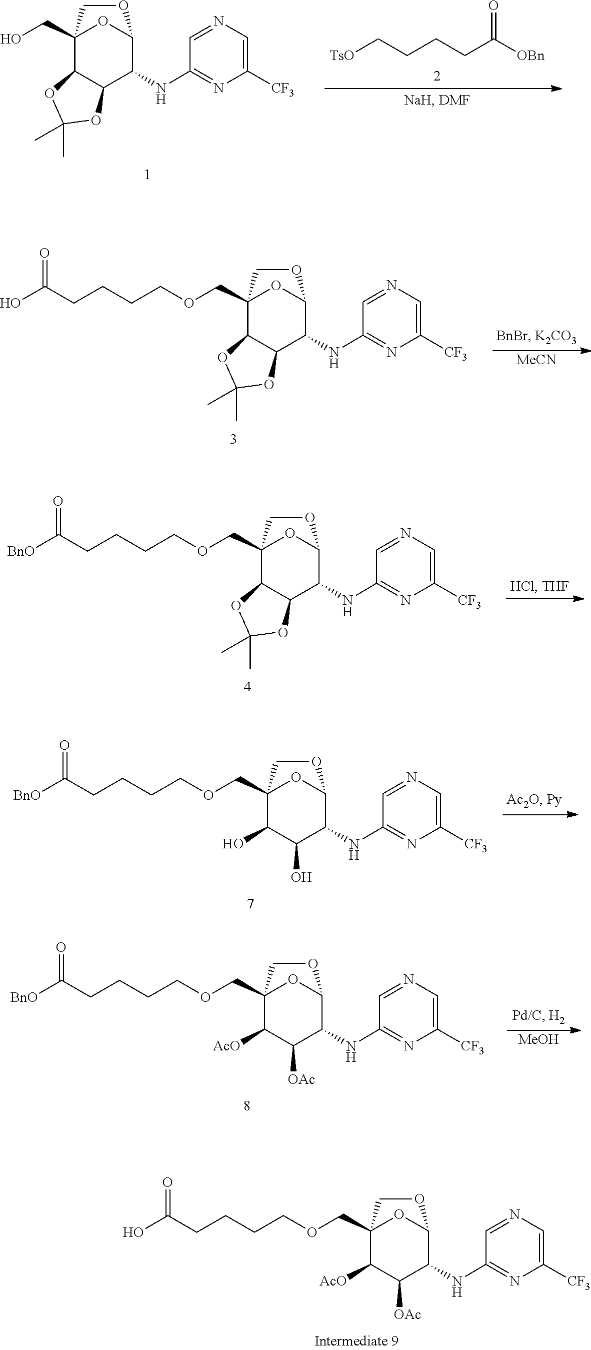

Step 1: To a solution of ((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methanol (1.00 eq) in DMF is added NaH (60% purity, 3.00 eq) at 0° C., the mixture is stirred at 25° C. for 0.5 hour, then benzyl 5-(tosyloxy)pentanoate (1.20 eq) is added, the resulting mixture is stirred at 60° C. for 4 hours under nitrogen atmosphere. The mixture is quenched with saturated solution of NH₄Cl, then concentrated under reduced pressure. The residue is purified by reverse phase to afford 5-(((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methoxy)pentanoic acid.

Step 2: To a solution of 5-(((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methoxy)pentanoic acid (1.00 eq) in MeCN is added K₂CO₃ (1.10 eq) and BnBr (1.00 eq), the mixture is stirred at 80° C. for 5 hours. The mixture is concentrated under reduced pressure. The residue is purified by silica gel chromatography to give benzyl 5-(((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methoxy)pentanoate.

Step 3: To a solution of benzyl 5-(((3aR,4S,7S,8R,8aR)-2,2-dimethyl-8-((6-(trifluoromethyl)pyrazin-2-yl)amino)tetrahydro-4,7-epoxy[1,3]dioxolo[4,5-d]oxepin-4(5H)-yl)methoxy)pentanoate (1.00 eq) in THF is added HCl (2M) at 0° C., the mixture is stirred at 25° C. for 10 hours under nitrogen atmosphere. The mixture is concentrated under reduced pressure. The residue is purified by reverse phase to afford benzyl 5-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)pentanoate.

Step 4: To a solution of benzyl 5-(((1S,2R,3R,4R,5S)-2,3-dihydroxy-4-((6-(trifluoromethyl)pyrazin-2-yl)amino)-6,8-dioxabicyclo[3.2.1]octan-1-yl)methoxy)pentanoate (1.00 eq) in pyridine is added Ac₂O (3.00 eq) at 0° C., the mixture is stirred at 25° C. for 10 hours under nitrogen atmosphere. The mixture is concentrated under reduced pressure. The residue is purified by reverse phase to afford (1S,2R,3R,4R,5S)-1-(((5-(benzyloxy)-5-oxopentyl)oxy)methyl)-4-((6-(trifluoromethyl)pyrazin-2-yl)amino)-6,8-dioxabicyclo[3.2.1]octane-2,3-diyl diacetate.

Step 5: To a solution of (1S,2R,3R,4R,5S)-1-(((5-(benzyloxy)-5-oxopentyl)oxy)methyl)-4-((6-(trifluoromethyl)pyrazin-2-yl)amino)-6,8-dioxabicyclo[3.2.1]octane-2,3-diyl diacetate (1.00 eq) in MeOH is added Pd/C (10% purity, 0.20 eq), the mixture is degassed and purged with hydrogen for several times, then is stirred at 25° C. for 2 hours under hydrogen atmosphere (15 psi). The mixture is filtered, the filtrate is concentrated under reduced pressure. The residue is purified by reverse phase to afford 5-(((1S,2R,3R,4R,5S)-2,3-diacetoxy-4-((6-(trifluoromethyl)pyrazin-2-yl)amino)-6,8-dioxabicyclo[3.2.1]octan-1-yl)methoxy)pentanoic acid (Intermediate 9).

### Preparation of 1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oic acid (Intermediate 10)

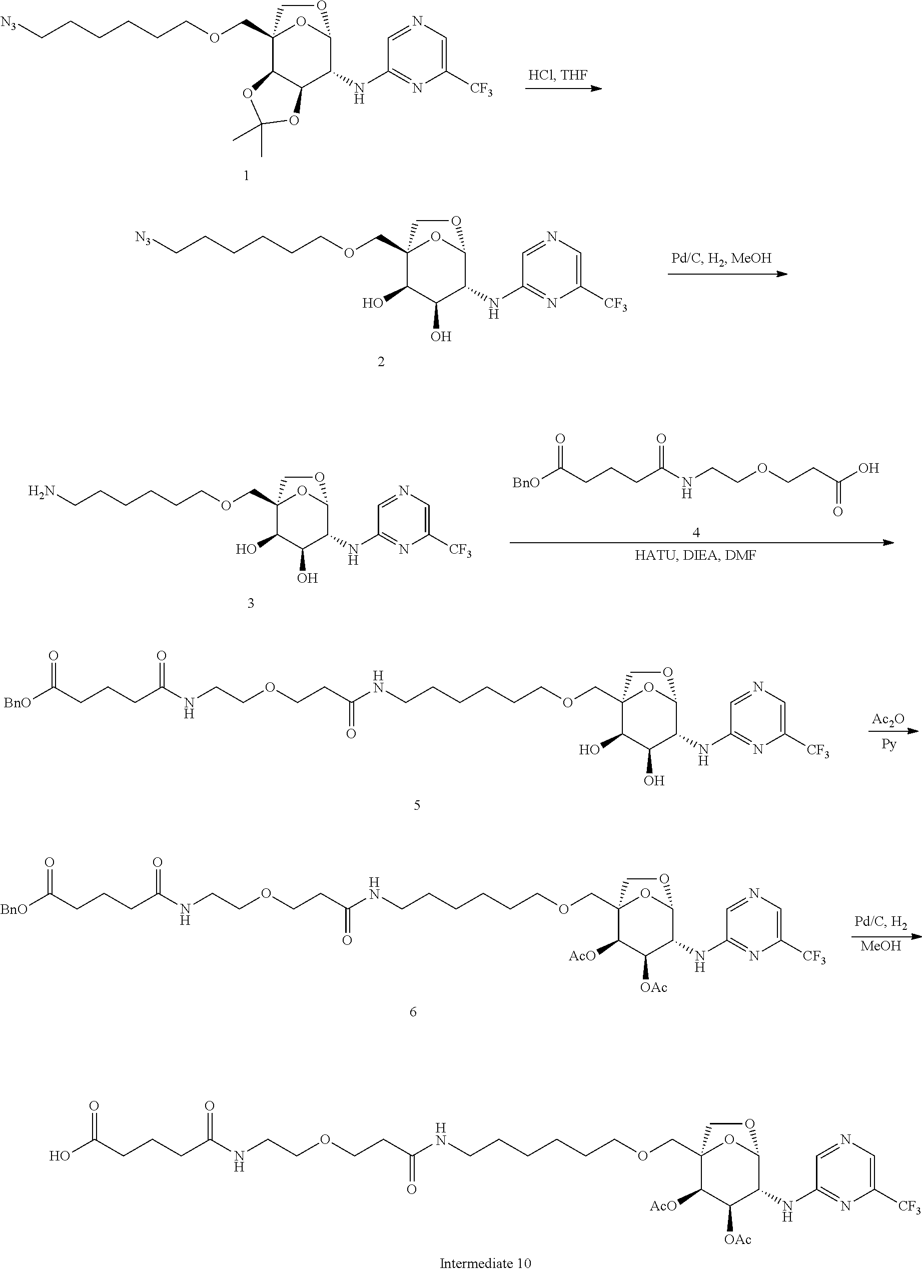

Step 1: To a solution of N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyrazin-2-amine (1.00 eq) in THF is added HCl (2M) at 0° C., the mixture is stirred at 25° C. for 12 hours under nitrogen atmosphere. The mixture is concentrated under reduced pressure. The residue is purified by reverse phase to afford (1S,2R,3R,4R,5S)-1-(((6-azidohexyl)oxy)methyl)-4-((6-(trifluoromethyl)pyrazin-2-yl)amino)-6,8-dioxabicyclo[3.2.1]octane-2,3-diol.

Step 2: To a solution of (1S,2R,3R,4R,5S)-1-(((6-azidohexyl)oxy)methyl)-4-((6-(trifluoromethyl)pyrazin-2-yl)amino)-6,8-dioxabicyclo[3.2.1]octane-2,3-diol (1.00 eq) in MeOH is added Pd/C (10% purity, 0.30 eq), the mixture is degassed and purged with hydrogen for several times, then stirred at 25° C. for 4 hours under hydrogen atmosphere (15 psi). The mixture is filtered, the filtrate is concentrated under reduced pressure. The residue is purified by reverse phase to afford (1S,2R,3R,4R,5S)-1-(((6-aminohexyl)oxy)methyl)-4-((6-(trifluoromethyl)pyrazin-2-yl)amino)-6,8-dioxabicyclo[3.2.1]octane-2,3-diol.

Step 3: To a solution of 3-(2-(5-(benzyloxy)-5-oxopentanamido)ethoxy)propanoic acid (1.20 eq) in DMF is added HATU (1.30 eq) and DIEA (4.00 eq), the mixture is stirred at 25° C. for 0.5 hour, then (1S,2R,3R,4R,5S)-1-(((6-aminohexyl)oxy)methyl)-4-((6-(trifluoromethyl)pyrazin-2-yl)amino)-6,8-dioxabicyclo[3.2.1]octane-2,3-diol (1.00 eq) is added, the resulting mixture is stirred at 25° C. for 6 hours. The mixture is diluted with water, extracted with EtOAc, the organic layer is washed with brine, dried over anhydrous sodium sulfate, filtered and is concentrated under reduced pressure. The residue is purified by silica gel chromatography to afford benzyl 1-((1S,2R,3R,4R,5S)-2,3-dihydroxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oate.

Step 4: To a solution of benzyl 1-((1S,2R,3R,4R,5S)-2,3-dihydroxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oate (1.00 eq) in pyridine is added Ac₂O (10.00 eq), the mixture is stirred at 25° C. for 8 hours. The mixture is diluted with water, extracted with EtOAc, the organic layer is washed with brine, dried over anhydrous sodium sulfate, filtered and concentrated under reduced pressure. The residue is purified by silica gel chromatography to afford (1S,2R,3R,4R,5S)-4-((6-(trifluoromethyl)pyrazin-2-yl)amino)-1-(3,7,14-trioxo-1-phenyl-2,11,22-trioxa-8,15-diazatricosan-23-yl)-6,8-dioxabicyclo[3.2.1]octane-2,3-diyl diacetate.

Step 5: To a solution of (1S,2R,3R,4R,5S)-4-((6-(trifluoromethyl)pyrazin-2-yl)amino)-1-(3,7,14-trioxo-1-phenyl-2,11,22-trioxa-8,15-diazatricosan-23-yl)-6,8-dioxabicyclo[3.2.1]octane-2,3-diyl diacetate (1.00 eq) in MeOH is added Pd/C (10% purity, 0.20 eq), the mixture is degassed and purged with hydrogen for several times, then is stirred at 25° C. for 3 hours under hydrogen atmosphere (15 psi). The mixture is filtered, the filtered cake is washed with MeOH three times, the combined filtrate is concentrated under reduced pressure. The residue is purified by reverse phase to afford 1-((1S,2R,3R,4R,5S)-2,3-diacetoxy-4-((6-(trifluoromethyl)pyrazin-2-yl)amino)-6,8-dioxabicyclo[3.2.1]octan-1-yl)-10,17-dioxo-2,13-dioxa-9,16-diazahenicosan-21-oic acid (Intermediate 10).

### Preparation of 15,15-bis((3-((6-(((1S,2R,3R,4R,5S)-2,3-diacetoxy-4-((6-(trifluoromethyl) pyridin-2-yl)amino)-6,8-dioxabicyclo[3.2.1]octan-1-yl)methoxy)hexyl)amino)-3-oxopropoxy) methyl)-1-((1S,2R,3R,4R,5S)-2,3-diacetoxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oic acid (Intermediate 11)

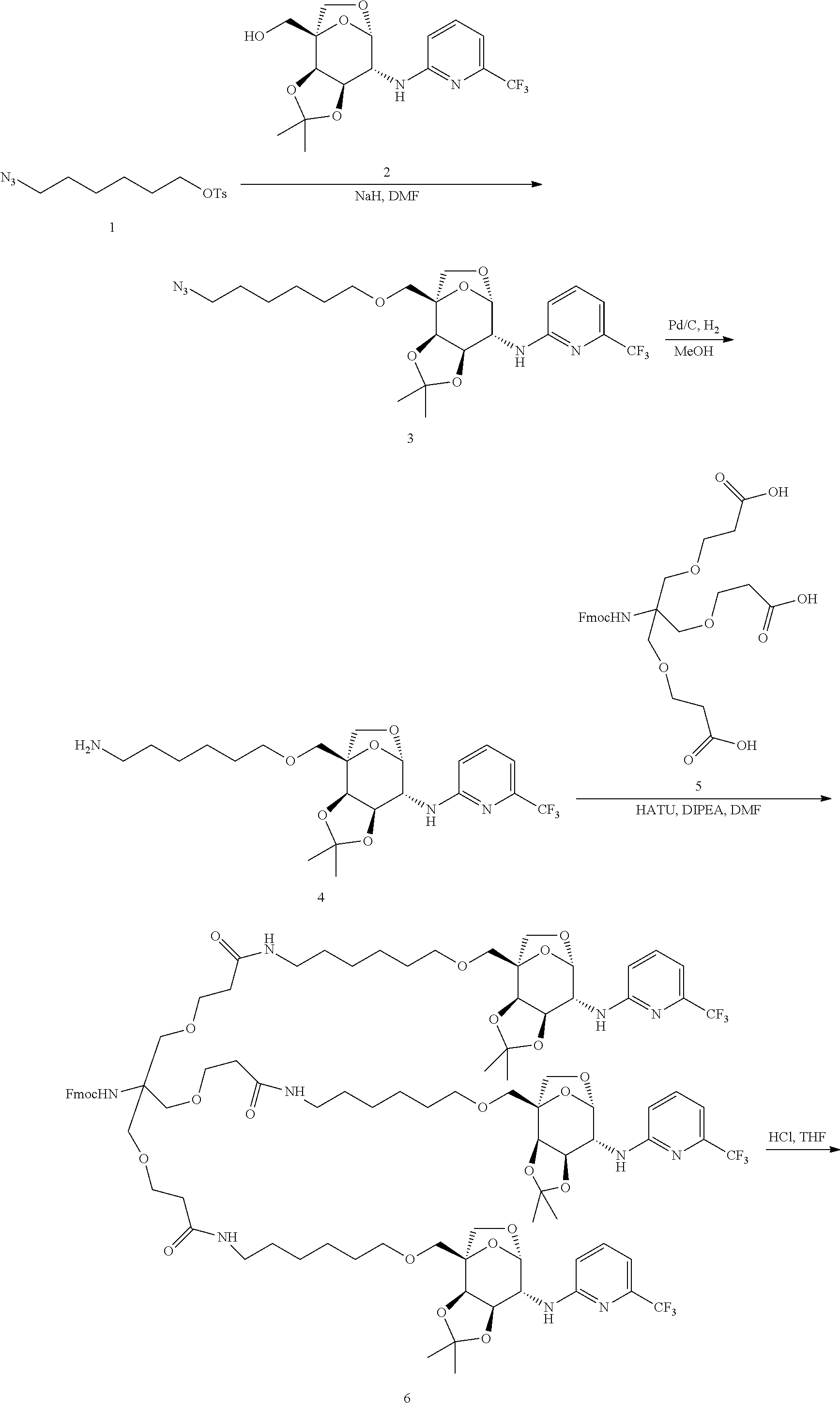

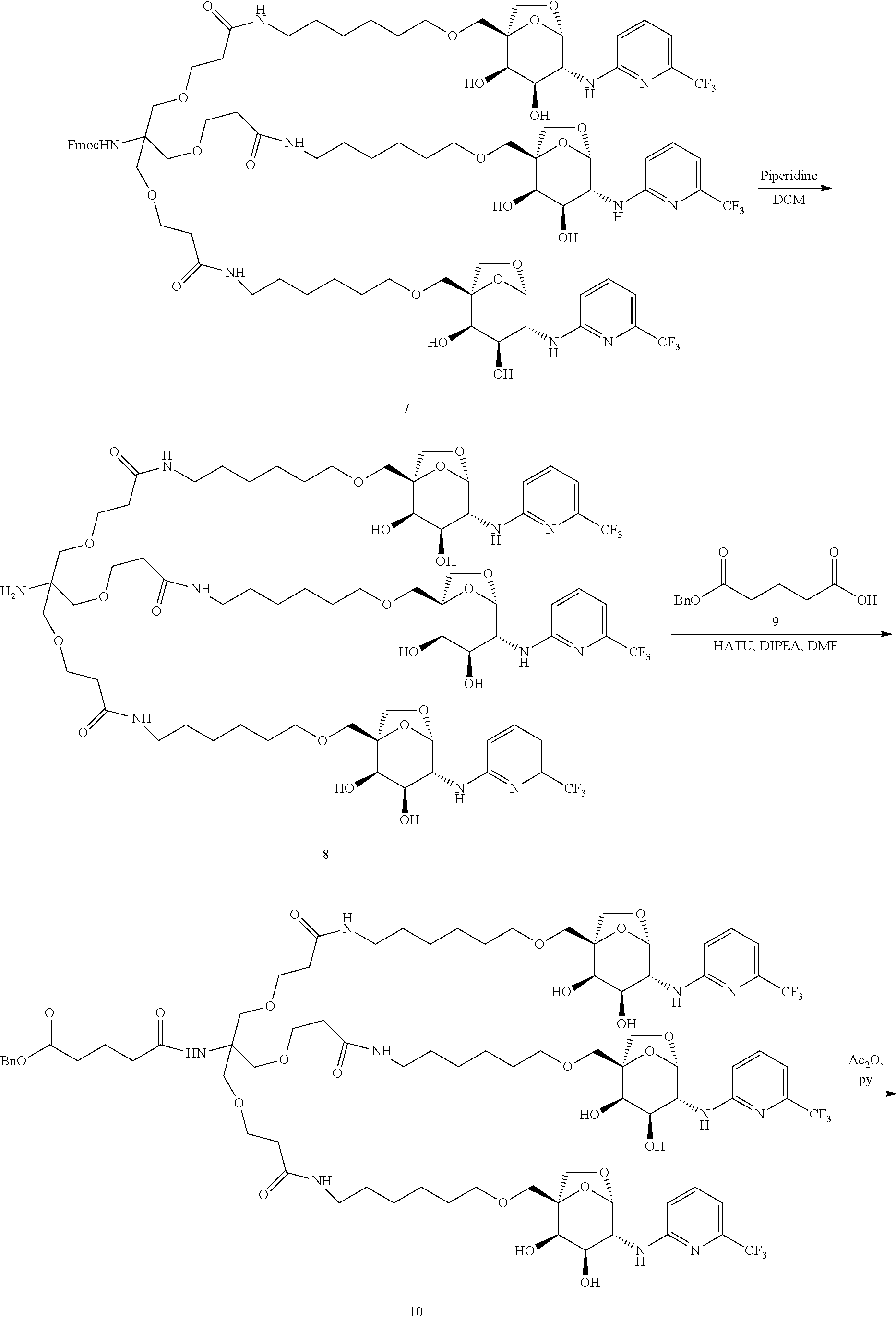

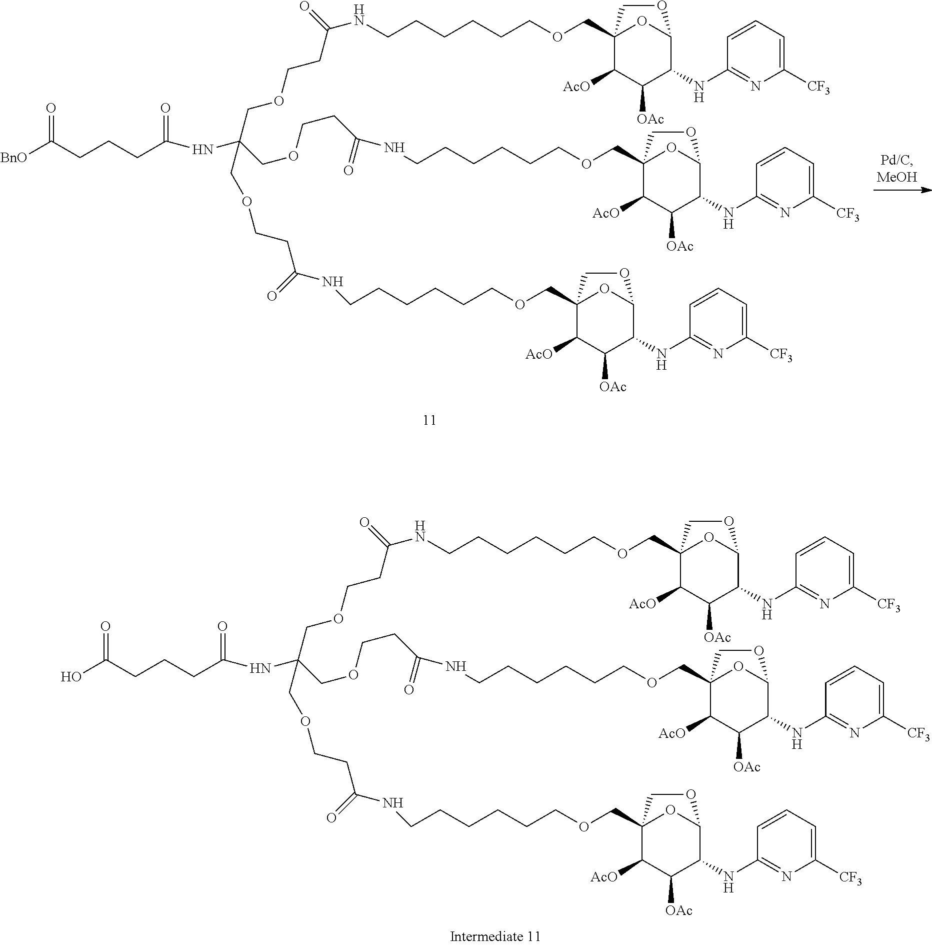

Step 1: To a solution of ((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methanol (1.00 eq) in DMF is added NaH (60% purity, 3.00 eq) at 0° C., the mixture is stirred at 25° C. for 0.5 hour, then 6-azidohexyl 4-methylbenzenesulfonate (1.00 eq) is added, the resulting mixture is stirred at 25° C. for 12 hours under nitrogen atmosphere. The mixture is quenched with saturated solution of NH₄Cl, then extracted with EtOAc, the organic layer is washed with brine, dried over anhydrous sodium sulfate, then concentrated under reduced pressure. The residue is purified by silica gel chromatography to afford N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyridin-2-amine.

Step 2: To a solution of N-((3aR,4S,7S,8R,8aR)-4-(((6-azidohexyl)oxy)methyl)-2,2-dimethylhexahydro-4,7-epoxy[1,3]dioxolo[4,5-d]oxepin-8-yl)-6-(trifluoromethyl)pyridin-2-amine (1.00 eq) in MeOH is added Pd/C (10% purity, 0.20 eq) and NH₃·H₂O (0.1 mL), the mixture is degassed and purged with hydrogen several times, then stirred at 25° C. for 1 hour. The mixture is filtered, and then the filtrate is concentrated under reduced pressure to afford N-((3aR,4S,7S,8R,8aR)-4-(((6-aminohexyl)oxy)methyl)-2,2-dimethylhexahydro-4,7-epoxy[1,3]dioxolo[4,5-d]oxepin-8-yl)-6-(trifluoromethyl)pyridin-2-amine.

Step 3: To a solution of 3,3′-((2-((((9H-fluoren-9-yl)methoxy)carbonyl)amino)-2-((2-carboxyethoxy)methyl)propane-1,3-diyl)bis(oxy))dipropionic acid (1.00 eq) in DMF is added HATU (4.50 eq) and DIEA (6.00 eq), the mixture is stirred at 25° C. for 0.5 hour, then N-((3aR,4S,7S,8R,8aR)-4-(((6-aminohexyl)oxy)methyl)-2,2-dimethylhexahydro-4,7-epoxy[1,3]dioxolo[4,5-d]oxepin-8-yl)-6-(trifluoromethyl)pyridin-2-amine (5.00 eq) is added, the resulting mixture is stirred at 25° C. for 10 hours. The mixture is concentrated under reduced pressure. The residue is purified by reverse phase to give (9H-fluoren-9-yl)methyl (1,29-bis((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)-15-((3-((6-(((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)methoxy)hexyl)amino)-3-oxopropoxy)methyl)-10,20-dioxo-2,13,17,28-tetraoxa-9,21-diazanonacosan-15-yl)carbamate.

Step 4: To a solution of (9H-fluoren-9-yl)methyl (1,29-bis((3aR,4S,7S,8R,8aR)-2,2-dimethyl-8-((6-(trifluoromethyl)pyridin-2-yl)amino)tetrahydro-4,7-epoxy[1,3]dioxolo[4,5-d]oxepin-4(5H)-yl)-15-((3-((6-(((3aR,4S,7S,8R,8aR)-2,2-dimethyl-8-((6-(trifluoromethyl)pyridin-2-yl)amino) tetrahydro-4,7-epoxy[1,3]dioxolo[4,5-d]oxepin-4(5H)-yl)methoxy)hexyl)amino)-3-oxopropoxy)methyl)-10,20-dioxo-2,13,17,28-tetraoxa-9,21-diazanonacosan-15-yl)carbamate (1.00 eq) in THF is added HCl (2M) at 0° C., the mixture is stirred at 25° C. for 10 hours under nitrogen atmosphere. The mixture is concentrated under reduced pressure. The residue is purified by reverse phase to afford (9H-fluoren-9-yl)methyl(15-((3-((6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl)amino)-3-oxopropoxy)methyl)-1,29-bis((1S,2R,3R,4R,5S)-2,3-dihydroxy-4-((6-(trifluoromethyl)pyridin-2-yl)amino)-6,8-dioxabicyclo[3.2.1]octan-1-yl)-10,20-dioxo-2,13,17,28-tetraoxa-9,21-diazanonacosan-15-yl)carbamate.

Step 5: To a solution of (9H-fluoren-9-yl)methyl (15-((3-((6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl) amino)-3-oxopropoxy)methyl)-1,29-bis((1S,2R,3R,4R,5S)-2,3-dihydroxy-4-((6-(trifluoromethyl) pyridin-2-yl)amino)-6,8-dioxabicyclo[3.2.1]octan-1-yl)-10,20-dioxo-2,13,17,28-tetraoxa-9,21-diazanonacosan-15-yl)carbamate (1.00 eq) in DCM is added Piperidine (2.00 eq), the mixture is stirred at 25° C. for 10 hours. The mixture is concentrated under reduced pressure. The residue is purified by reverse phase to afford 3,3′-((2-amino-2-((3-((6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl) amino)-3-oxopropoxy)methyl)propane-1,3-diyl)bis(oxy))bis(N-(6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl)propanamide).

Step 6: To a solution of 5-(benzyloxy)-5-oxopentanoic acid (1.00 eq) in DMF is added HATU (1.50 eq) and DIEA (2.00 eq), the mixture is stirred at 25° C. for 0.5 hour, then 3,3′-((2-amino-2-((3-((6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl)amino)-3-oxopropoxy)methyl)propane-1,3-diyl) bis(oxy))bis(N-(6-(((1S,2R,3R,4R,5S)-2,3-dihydroxy-4-((6-(trifluoromethyl)pyridin-2-yl)amino) -6,8-dioxabicyclo[3.2.1]octan-1-yl)methoxy)hexyl)propanamide) (1.10 eq) is added, the resulting mixture is stirred at 25° C. for 10 hours. The mixture is concentrated under reduced pressure. The residue is purified by reverse phase to give benzyl 15,15-bis((3-((6-(((1S,2R,3R,4R,5S)-2,3-dihydroxy-4-((6-(trifluoromethyl)pyridin-2-yl)amino)-6,8-dioxabicyclo [3.2.1]octan-1-yl)methoxy)hexyl)amino)-3-oxopropoxy)methyl)-1-((1S,2R,3R,4R,5S)-2,3-dihydroxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oate.

Step 7: To a solution of benzyl 15,15-bis((3-((6-(((1S,2R,3R,4R,5S)-2,3-dihydroxy-4-((6-(trifluoromethyl)pyridin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-dihydroxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oate (1.00 eq) in Pyridine is added Ac₂O (10.0 eq) at 0° C., the mixture is stirred at 25° C. for 10 hours under nitrogen atmosphere. The mixture is concentrated under reduced pressure. The residue is purified by reverse phase to afford benzyl 15,15-bis((3-((6-(((1S,2R,3R,4R,5S)-2,3-diacetoxy-4-((6-(trifluoromethyl)pyridin-2-yl)amino)-6,8-dioxabicyclo[3.2.1]octan-1-yl)methoxy)hexyl) amino)-3-oxopropoxy)methyl)-1-((1S,2R,3R,4R,5S)-2,3-diacetoxy-4-((6-(trifluoromethyl) pyridine-2-yl)amino)-6,8-dioxabicyclo[3.2.1]octan-1-yl)-10,17-dioxo-2,13-dioxa-9,16-diazahenicosan-21-oate.

Step 8: To a solution of benzyl 15,15-bis((3-((6-(((1S,2R,3R,4R,5S)-2,3-diacetoxy-4-((6-(trifluoromethyl)pyridin-2-yl)amino)-6,8-dioxabicyclo[3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oate (1.00 eq) in MeOH is added Pd/C (10% purity, 0.20 eq), the mixture is degassed and purged with hydrogen several times, then is stirred at 25° C. for 2 hours under hydrogen atmosphere (15 psi). The mixture is filtered, the filtrate is concentrated under reduced pressure. The residue is purified by reverse phase to afford 15,15-bis((3-((6-(((1S,2R,3R,4R,5S)-2,3-diacetoxy-4-((6-(trifluoromethyl)pyridin-2-yl)amino)-6,8-dioxabicyclo [3.2.1]octan-1-yl)methoxy)hexyl)amino)-3-oxopropoxy)methyl)-1-((1S,2R,3R,4R,5S)-2,3-diacetoxy-4-((6-(trifluoromethyl)pyridin-2-yl)amino)-6,8-dioxabicyclo[3.2.1]octan-1-yl)-10,17-dioxo-2,13-dioxa-9,16-diazahenicosan-21-oic acid (Intermediate 11).

### Example 2. Synthesis of Therapeutic Oligonucleotide Delivering Compound 1

### Step 1. Tridentate Synthesis

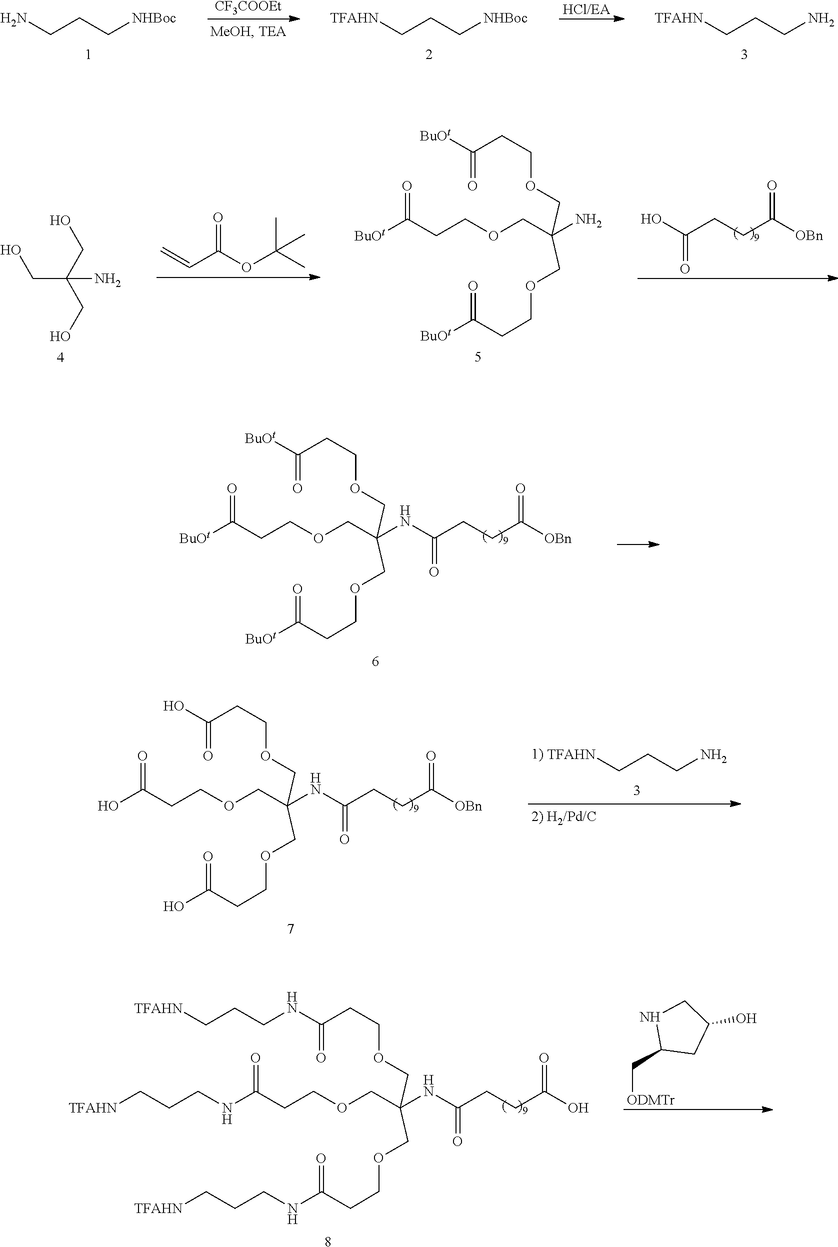

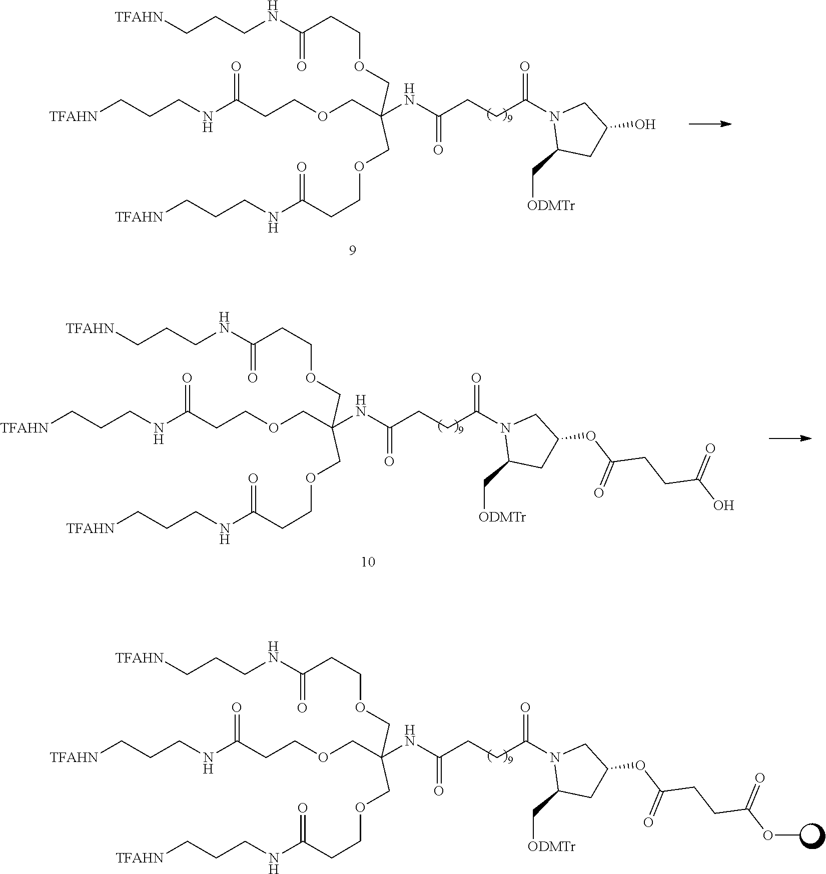

### Step 2. Synthesis of Therapeutic Oligonucleotide Delivering Compound 1

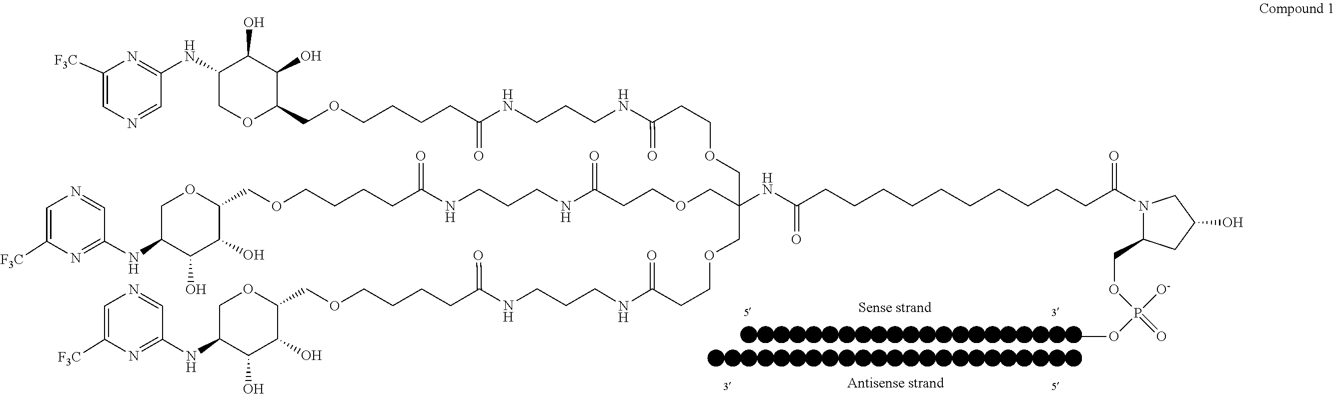 [SMILES: COP(=O)([O-])OC[C@@H]1C[C@@H](O)CN1C(=O)CCCCCCCCCCC(=O)NC(COCCC(=O)NCCCNC(=O)CCCCOC[C@H]1OC[C@H](Nc2cncc(C(F)(F)F)n2)[C@@H](O)[C@H]1O)(COCCC(=O)NCCCNC(=O)CCCCOC[C@H]1OC[C@H](Nc2cncc(C(F)(F)F)n2)[C@@H](O)[C@H]1O)COCCC(=O)NCCCNC(=O)CCCCOC[C@H]1OC[C@H](Nc2cncc(C(F)(F)F)n2)[C@@H](O)[C@H]1O]

(SEQ ID NO: 11)
Sense Strand Sequence =
5′- c*a*gaaaGaGuGuCuCaucuua-3′
(SEQ ID NO: 12)
Antisense Strand Sequence =
5′- u*A*AGaUgAgAcAcUcUuUcUg*g*u-3′

Step 2A. Synthesis of the amine sense strand and the antisense strand is described in the synthesis of Compound 2.

Step 2B. Conjugation of the amine sense strand with Intermediate 2

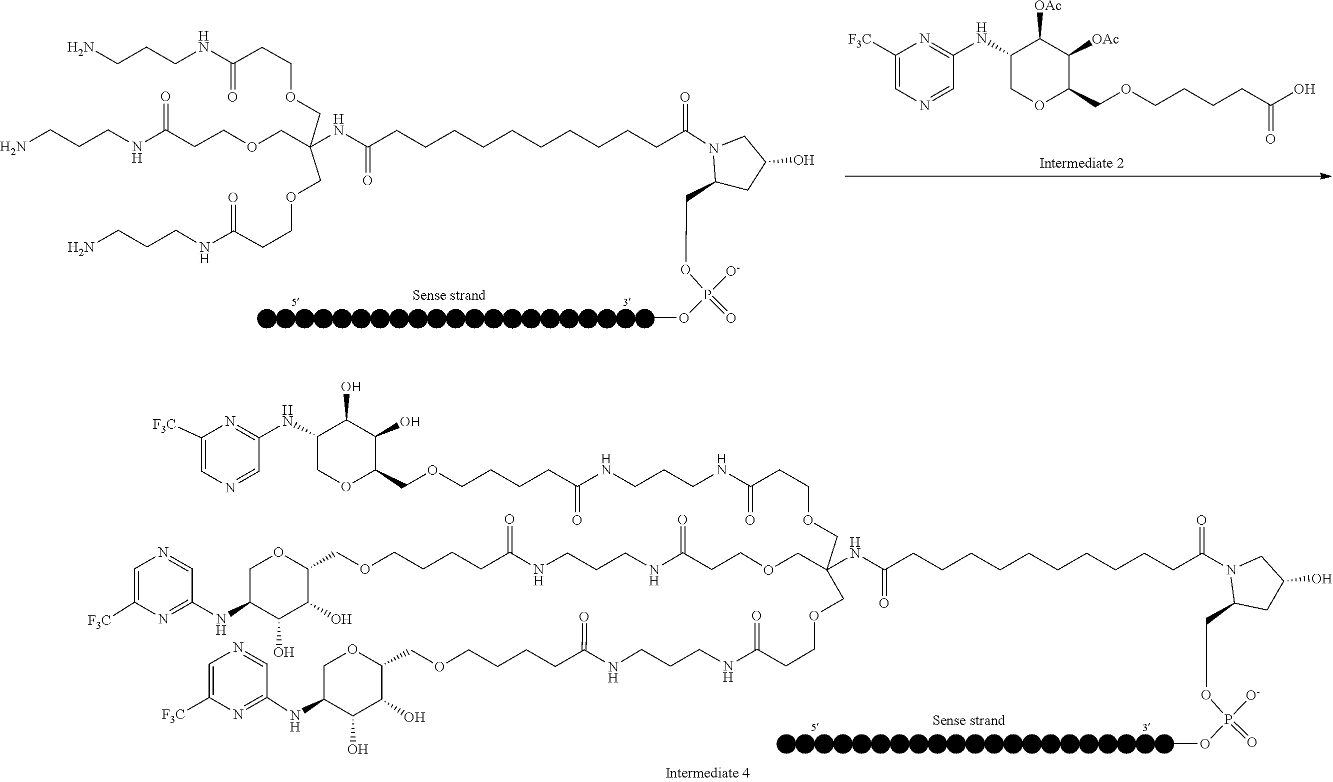 [SMILES: COP(=O)([O-])OC[C@@H]1C[C@@H](O)CN1C(=O)CCCCCCCCCCC(=O)NC(COCCC(=O)NCCCN)(COCCC(=O)NCCCN)COCCC(=O)NCCCN ; CC(=O)O[C@@H]1[C@H](OC(C)=O)[C@@H](Nc2cncc(C(F)(F)F)n2)CO[C@@H]1COCCCCC(=O)O ; COP(=O)([O-])OC[C@@H]1C[C@@H](O)CN1C(=O)CCCCCCCCCCC(=O)NC(COCCC(=O)NCCCNC(=O)CCCCOC[C@H]1OC[C@H](Nc2cncc(C(F)(F)F)n2)[C@@H](O)[C@H]1O)(COCCC(=O)NCCCNC(=O)CCCCOC[C@H]1OC[C@H](Nc2cncc(C(F)(F)F)n2)[C@@H](O)[C@H]1O)COCCC(=O)NCCCNC(=O)CCCCOC[C@H]1OC[C@H](Nc2cncc(C(F)(F)F)n2)[C@@H](O)[C@H]1O]

To a solution of Intermediate 2 acid (13 mg, 5 eq.) in DMF (200 ul) was added TFA-O—PFP (56 uL, 64 eq.) and followed by DIPEA (60 uL, 64 eq.) at 25° C. The tube was shaken for 1~2 h at 25° C. The reaction was quenched with H₂O. The resulting mixture was diluted with DMF (800 ul), followed by addition of oligo-amine solution (40 mg in 1 mL, 10×PBS, pH 7.4). The tube was shaken at 25° C. for 16 h; the reaction mixture was treated by 28% NH₄₀H (2 mL) for 5 h, 48.79% DP by LCMS.

Purified by IP—RP-HPLC, obtained TEA salt (Intermediate 4): 18 mg, 93.57% purity.

### Purification Conditions:

Waters Xbridge BEH C18 10 μm, 19*250 mm;
Mobile Phase A: 100 mM HFIP-5 mM TEA in water
Mobile Phase B: 80% ACN; Column temperature: 45° C.

Conversion to sodium salt: Intermediate 4 TEA salt was dissolved in 1 mL (1M, NaOAc) at rt. for 5 min, diluted with EtOH (10 mL) and cooled to −40° C. for 0.5 h. The resulting precipitate was centrifuged, and the collected solid was washed with EtOH (10 mL*2), dissolved in Milli Q. water, and lyophilized to give sodium salt 15 mg (by OD), 98.67% purity.

### Step 3. Duplex Formation

To form siRNA duplex, equal molar amounts of antisense strand Intermediate 4 was mixed then heated to 95° C. for 2 minutes. The resulting mixture was lyophilized to give final siRNA duplex.

Obtained siRNA duplex: 27 mg (by OD), 99.17% purity.

LCMS conditions (for analysis of single strands):
Instrument: LCMS—Agilent 1260 Prime-G6230
Column: XBridge® Prmier Oligo BEH C18 130 Å 2.5 m, 2.1*100 mm
Mobile Phase A: 100 mM HFIP-5 mM TEA in water
Mobile Phase B: 80% ACN; Column temperature: 45° C.

### Ion Mode: ESI Negative

TABLE 3
Compound 1 characterization data

| Compound ID | Calcd.MW | Observed MW |
|---|---|---|
| Compound 1 | SS: 9000.66 | SS: 9000.76 |
| | AS: 7563.87 | AS: 7563.59 |

### Example 3. Synthesis of Therapeutic Oligonucleotide Delivering Compound 2

### Step 1. Monodentate Synthesis

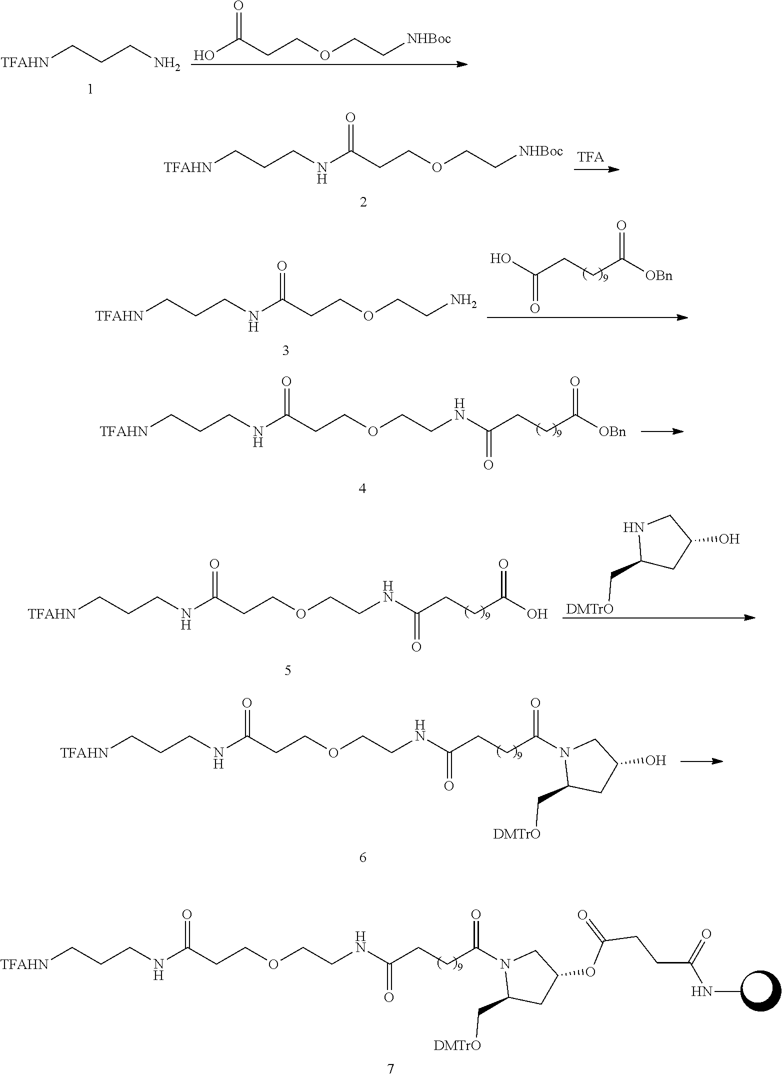

### Step 2. Synthesis of Therapeutic Oligonucleotide Delivering Compound 2

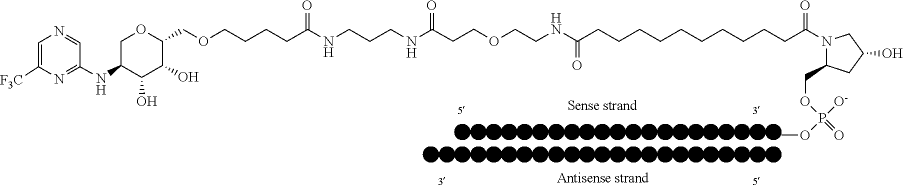 [SMILES: COP(=O)([O-])OC[C@@H]1C[C@@H](O)CN1C(=O)CCCCCCCCCCC(=O)NCCOCCC(=O)NCCCNC(=O)CCCCOC[C@H]1OC[C@H](Nc2cncc(C(F)(F)F)n2)[C@@H](O)[C@H]1O]

Sense Strand:
(SEQ ID NO: 11)
5′-c*a*gaaaGaGuGuCuCaucuua-3′
Antisense Strand:
(SEQ ID NO: 12)
5′- u*A*AGaUgAgAcAcUcUuUcUg*g*u -3′
Abbreviation:
Lower case (e.g. a/u/g/c) = 2′-OMe;
upper case (e.g. A/U/G/C) = 2′-F;
* = phosphorothioate linkage (PS bond)

### Step 2A. Synthesis of the Amine Sense Strand

TABLE 4
Oligonucleotide synthesis conditions

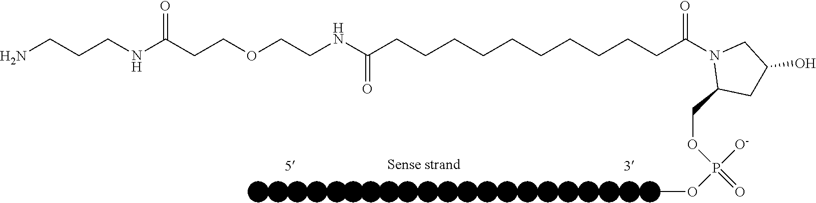 [SMILES: COP(=O)([O-])OC[C@@H]1C[C@@H](O)CN1C(=O)CCCCCCCCCCC(=O)NCCOCCC(=O)NCCCN]

Sense Strand: 5′-c*a*gaaaGaGuGuCuCaucuua-3′ (SEQ ID NO: 11)

| | |
|---|---|
| Synthesizer | MerMade 12 |
| CPG | SS: Mono-amino prolinol CPG (139 umol/g) |
| | AS: universal CPG (500 A, 103 umol/g) |
| Scale (ummol) | SS: 24 umol * 2 AS: 24 umol * 2 |
| Coupling | 24 umol, 0.05M amidite, 2 eq. each time; 0.25M ETT (2 mL each |
| | time); repeated 4 times, 8 min in total. |
| | Monomer: 2′-OMe-A(Bz), 2′-OMe-U, 2′-OMe-G(iBu), 2′-OMe-C(Ac); |
| | 2′-F-A(Bz), 2′-F—U, 2′-F-G(iBu), 2′-F—C(Ac) |
| Oxidation | 0.02M iodine solution in MeCN/pyridine/H₂O |
| Sulfurization | ADTT (0.2M, 20 eq.) in pyridine, 7 min |
| Cleavage and | 20% DEA (50 mL), 30 mins; 28% NH₄OH/EtOH = 3:1, 45° C., 20 h. |
| deprotection | |
| (C&D) | |
| Purification | Waters Xbridge BEH C18 10 um, 19 * 250 mm; |
| by IP-RP-HPLC | Mobile Phase A: 100 mM HFIP-5 mM TEA in water |
| | Mobile Phase B: 80% ACN; Column temperature: 45° C. |
| Result | SS: purified by IP-RP-HPLC, obtained TEA salt 50 mg, 100% purity |
| | AS: purified by IP-RP-HPLC, obtained TEA salt: 60 mg, 100% |
| | purity; converted to Na salt, obtained Na salt 55 mg (by OD), |
| | 99.19% purity |

### Step 2B. Synthesis of the Antisense Strand

TABLE 5
Oligonucleotide synthesis conditions
Antisense Strand: 5′-u*A*AGaUgAgAcAcUcUuUcUg*g*u-3′ (SEQ ID NO: 12)

| | |
|---|---|
| Synthesizer | MerMade 12 |
| CPG | SS: Mono-amino prolinol CPG (139 umol/g) |
| | AS: universal CPG (500A, 103 umol/g) |
| Scale (ummol) | SS: 24 umol*2 AS: 24 umol*2 |
| Coupling | 24 umol, 0.05 M amidite, 2 eq. each time; 0.25 M ETT (2 mL each |
| | time); repeated 4 times, 8 min in total. |
| | Monomer: 2′-OMe-A(Bz), 2′-OMe-U, 2′-OMe-G(iBu), 2′-OMe- |
| | C(Ac); 2′-F-A(Bz), 2′-F-U, 2′-F-G(iBu), 2′-F-C(Ac) |
| Oxidation | 0.02 M iodine solution in MeCN/pyridine/H2O |
| Sulfurization | ADTT (0.2 M, 20 eq.) in pyridine, 7 min |
| Cleavage and | 20% DEA (50 mL), 30 mins; 28% NH4OH/EtOH = 3:1, 45° C., 20 h. |
| deprotection | |
| (C&D) | |
| Purification | Waters Xbridge BEH C18 10 um, 19*250 mm; |
| by IP-RP-HPLC | Mobile Phase A: 100 mM HFIP-5 mM TEA in water |
| | Mobile Phase B: 80% ACN; Column temperature: 45° C. |
| Result | SS: purified by IP-RP-HPLC, obtained TEA salt 50 mg, 100% |
| | purity |
| | AS: purified by IP-RP-HPLC, obtained TEA salt: 60 mg, 100% |
| | purity; converted to Na salt, obtained Na salt 55 mg (by OD), |
| | 99.19% purity |

Step 2C. Conjugation of the Amine Sense Strand with Intermediate 2

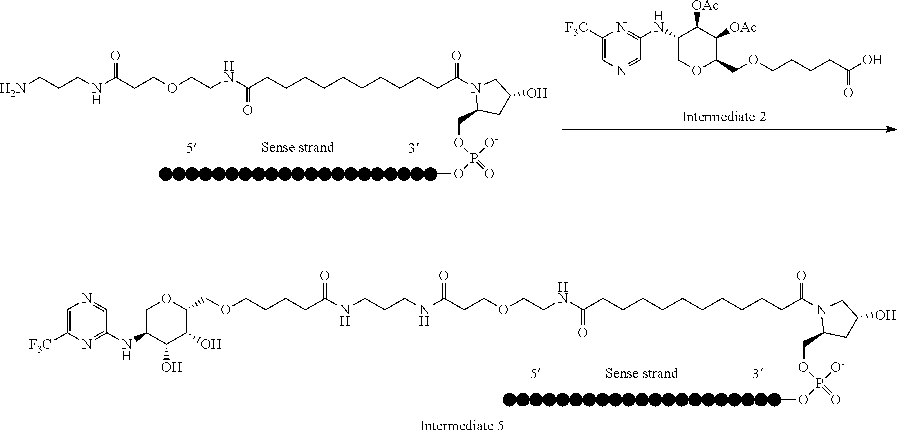 [SMILES: COP(=O)([O-])OC[C@@H]1C[C@@H](O)CN1C(=O)CCCCCCCCCCC(=O)NCCOCCC(=O)NCCCN ; CC(=O)O[C@@H]1[C@H](OC(C)=O)[C@@H](Nc2cncc(C(F)(F)F)n2)CO[C@@H]1COCCCCC(=O)O ; COP(=O)([O-])OC[C@@H]1C[C@@H](O)CN1C(=O)CCCCCCCCCCC(=O)NCCOCCC(=O)NCCCNC(=O)CCCCOC[C@H]1OC[C@H](Nc2cncc(C(F)(F)F)n2)[C@@H](O)[C@H]1O]

To a solution of Intermediate 2 acid (13 mg, 5 eq.) in DMSO/THF (0.16 mL), was added HATU (40 mg, 20 eq.), DIEA (71 uL, 80 eq.), the resulting mixture was stirred for 30 min at 25° C.; then diluted with DMSO/THF (0.8 mL, v/v=1:1), followed by addition of another solution of Oligo-NH₂ (1 eq. 40 mg) in Milli Q water (1.2 mL), DIEA (71 uL, 80.0 eq). The resulting solution was stirred at 25° C. for 2 h; the reaction mixture was treated by 28% NH₄₀H (2 mL) for 5 h, 84.24% DP by LCMS.

Purified by IP—RP-HPLC, obtained TEA salt: 29 mg.

### Purification Conditions:

Waters Xbridge BEH C18 10 μm, 19*250 mm;
Mobile Phase A: 100 mM HFIP-5 mM TEA in water
Mobile Phase B: 80% ACN; Column temperature: 45° C.

Conversion to sodium salt: Intermediate 5 TEA salt was dissolved in 1.5 mL (1M, NaOAc) at rt. for 5 min, diluted with EtOH (20 mL) and cooled to −40° C. for 0.5 h. The resulting precipitate was centrifuged, and the collected solid was washed with EtOH (10 mL*2), dissolved in Milli Q. water, and lyophilized to give Intermediate 5 sodium salt: 27 mg (by OD).

### Step 4. Duplex Formation

To form siRNA duplex, equal molar amounts of antisense strand and Intermediate 5 was mixed then heated to 95° C. for 2 minutes. The resulting mixture was lyophilized to give final siRNA duplex (Compound 2). Obtained siRNA duplex: 27 mg (by OD), 97.45% purity.

LCMS conditions (for analysis of single strands):
Instrument: LCMS—Agilent 1260 Prime-G6230
Column: XBridge® Prmier Oligo BEH C18 130 Å 2.5 m, 2.1*100 mm
Mobile Phase A: 100 mM HFIP-5 mM TEA in water
Mobile Phase B: 80% ACN; Column temperature: 45 oC.

### Ion Mode: ESI Negative

TABLE 6
Compound 2 characterization data

| Compound ID | Calcd.MW | Observed MW |
|---|---|---|
| Compound 2 | SS: 7901.56 | SS: 7901.45 |
| | AS: 7563.87 | AS: 7563.59 |

### Example 4. Synthesis of Therapeutic Oligonucleotide Delivering Compound 3

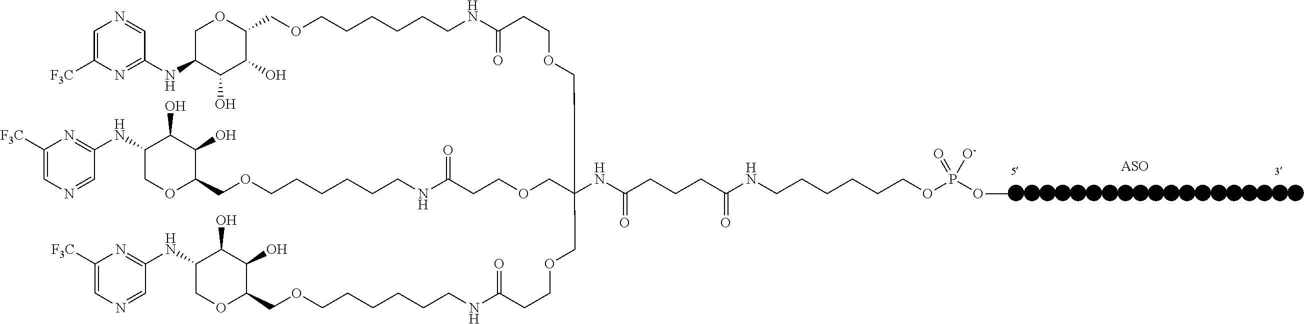 [SMILES: COP(=O)([O-])OCCCCCCNC(=O)CCCC(=O)NC(COCCC(=O)NCCCCCCOC[C@H]1OC[C@H](Nc2cncc(C(F)(F)F)n2)[C@@H](O)[C@H]1O)(COCCC(=O)NCCCCCCOC[C@H]1OC[C@H](Nc2cncc(C(F)(F)F)n2)[C@@H](O)[C@H]1O)COCCC(=O)NCCCCCCOC[C@H]1OC[C@H](Nc2cncc(C(F)(F)F)n2)[C@@H](O)[C@H]1O]

Sequence:
(SEQ ID NO: 38)
5′-(moeT)*(moeG)(moe5C)(moeT)(moe5C)d5C*dG*dT*
dT*dG*dG*dT*dG*d5C*dT*(moeT)(moeG)(moeT)*
(moeT)*(moe5C)-3′
Abbreviations used in Sequence:
(moeX) = 2′-O-MOE protected nucleotide X;
dX = deoxy (DNA) nucleotide X);
* = phosphorothioate linkage (PS bond)

### Step 1. Synthesis of the Oligo Amine

TABLE 7
Oligonucleotide synthesis conditions

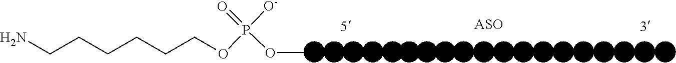

| | |
|---|---|
| Synthesizer | Mermade 12 |
| CPG | universal CPG (500 A) |
| Scale (umol) | Parent Oligo synthesis: 100 umol * 2 |
| Coupling | 100 umol scale, 0.1M amidite, 2.5 eq. each time; 0.25M ETT (5 mL each |
| | time); repeated 2 times, 12 min in total. |
| | Monomer: 2′-MOE-5C(Bz), 2′-MOE-G(iBu), 2′-MOE-T, d5C(Bz), dG(dmf), dT |
| Oxidation | 0.02M iodine solution in MeCN/pyridine/H₂O |
| Sulfurization | ADTT (0.2M, 20 eq.) in pyridine, 6 min |
| Cleavage and | 20% DEA (50 mL), 30 mins; 28% NH₄OH/EtOH = 3:1, 45° C., 20 h. |
| deprotection | |
| (C&D) | |
| Purification | Waters Xbridge BEH C18 10 um, 19 * 250 mm; |
| by IP-RP- | Mobile Phase A: 100 mM HFIP-5 mM TEA in water |
| HPLC | Mobile Phase B: 80% ACN; Column temperature: 45° C. |
| Result | Purified by IP-RP-HPLC, obtained TEA salt: 220 mg, 100% purity |

Step 2. 5F-Conjugation of the Oligo Amine with Intermediate 1

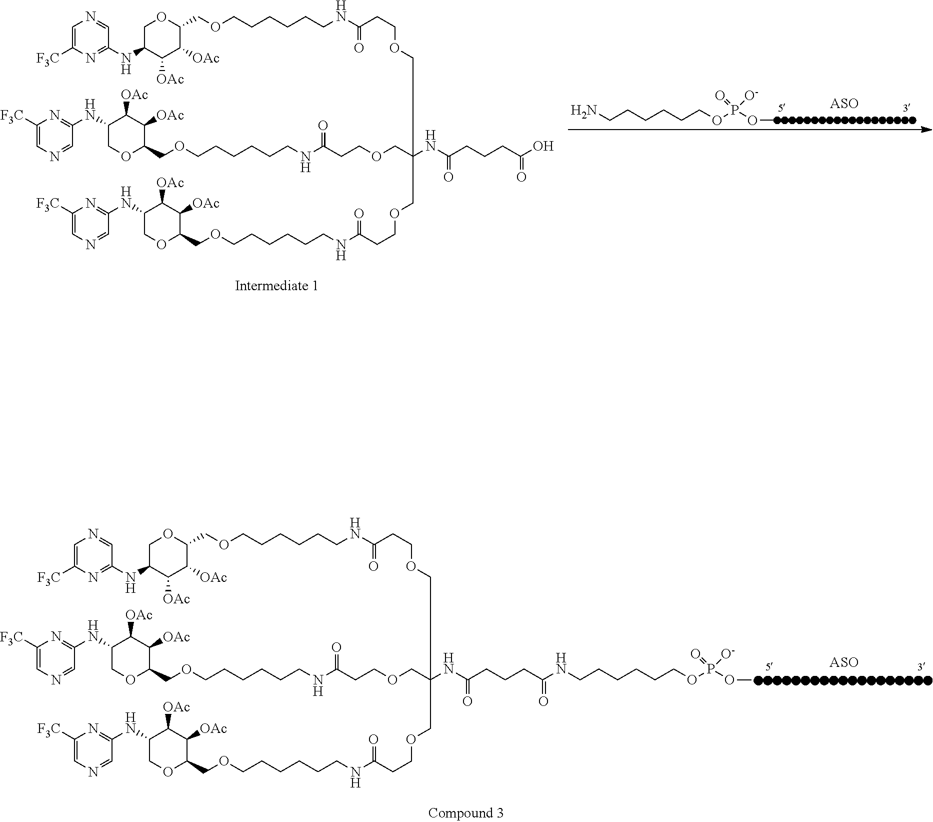

To a solution of Intermediate 1 acid (38 mg, 5 eq.) in DMSO/THF (0.4 mL,v/v=1:1), was added HATU (24 mg, 20 eq.), DIEA (44 uL, 80 eq.), the resulting mixture was stirred for 30 min at 25° C.; then diluted with DMSO/THF (0.6 mL, v/v=1:1), followed by addition of another solution of the Oligo-NH₂ (1 eq. 30 mg) in Milli Q water (1.2 mL), DIEA (44 uL, 80.0 eq). The resulting solution was stirred at 25° C. for 2 h, the reaction mixture was treated by 28% NH₄₀H (2 mL) for 5 h, 60% DP by LCMS.

Purified by IP—RP-HPLC, obtained TEA salt: 27 mg, 97.11% purity.

### Purification Condition:

Waters Xbridge BEH C18 10 μm, 19*250 mm;
Mobile Phase A: 100 mM HFIP-5 mM TEA in water
Mobile Phase B: 80% ACN; Column temperature: 45° C.

Converted to sodium salt: The TEA salt was dissolved in 1.5 mL (1M, NaOAc) at rt. for 5 min, diluted with EtOH (20 mL) and cooled to −40° C. for 0.5 h. The resulting precipitate was centrifuged, and the collected solid was washed with EtOH (10 mL*2), dissolved in Milli Q. water, and lyophilized to give Compound 3 sodium salt: 25.1 mg (by OD), 95.51% purity.

### LCMS Condition:

Instrument: LCMS—Agilent 1260 Prime-G6230
Column: XBridge® Prmier Oligo BEH C18 130 Å 2.5 m, 2.1*100 mm
Mobile Phase A: 100 mM HFIP-5 mM TEA in water
Mobile Phase B: 80% ACN; Column temperature: 45° C.

### Ion Mode: ESI Negative

TABLE 8
Compound 3 characterization data

| Compound ID | Calcd.MW | Observed MW |
|---|---|---|
| Compound 3 | 8899.46 | 8900.54 |

### Example 5. Synthesis of Therapeutic Oligonucleotide Delivering Compound 4

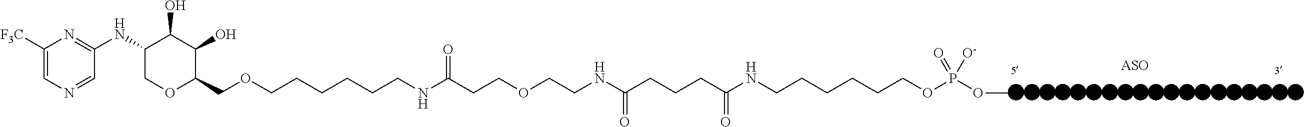 [SMILES: COP(=O)([O-])OCCCCCCNC(=O)CCCC(=O)NCCOCCC(=O)NCCCCCCOC[C@H]1OC[C@H](Nc2cncc(C(F)(F)F)n2)[C@@H](O)[C@H]1O]

Sequence:
(SEQ ID NO: 38)
5′-(moeT)*(moeG)(moe5C)(moeT)(moe5C)d5C*dG*
dT*dT*dG*dG*dT*dG*d5C*dT*(moeT)(moeG)(moeT)*
(moeT)*(moe5C)-3′
Abbreviations used in Sequence:
(moeX) = 2′-O-MOE protected nucleotide X;
dX = deoxy (DNA) nucleotide X);
* = phosphorothioate linkage (PS bond)

### Step 1. Synthesis of Oligonucleotide Amine

TABLE 9
Oligonucleotide synthesis conditions

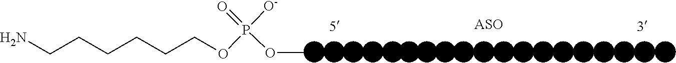

| | |
|---|---|
| Synthesizer | MerMade 12 |
| CPG | universal CPG (500 A) |
| Scale (umol) | 100 umol * 2 |
| Coupling | 0.1M amidite, 2.5 eq. each time; 0.25M ETT (5 mL each time); repeated |
| | 2 times, 12 min in total. |
| | Monomer: 2′-MOE-5C(Bz), 2′-MOE-G(iBu), 2′-MOE-T, d5C(Bz), dG(dmf), dT |
| Oxidation | 0.02M iodine solution in MeCN/pyridine/H₂O |
| Sulfurization | ADTT (0.2M, 20 eq.) in pyridine, 6 min |
| Cleavage and | 20% DEA (50 mL), 30 mins; 28% NH₄OH/EtOH = 3:1, 45° C., 20 h. |
| deprotection | |
| (C&D) | |
| Purification | Waters Xbridge BEH C18 10 um, 19 * 250 mm; |
| by IP-RP- | Mobile Phase A: 100 mM HFIP-5 mM TEA in water |
| HPLC | Mobile Phase B: 80% ACN; Column temperature: 45° C. |
| Result | Purified by IP-RP-HPLC, obtained TEA salt: 220 mg, 100% purity |

Step 2. Conjugation of the Oligo Amine with Intermediate 3

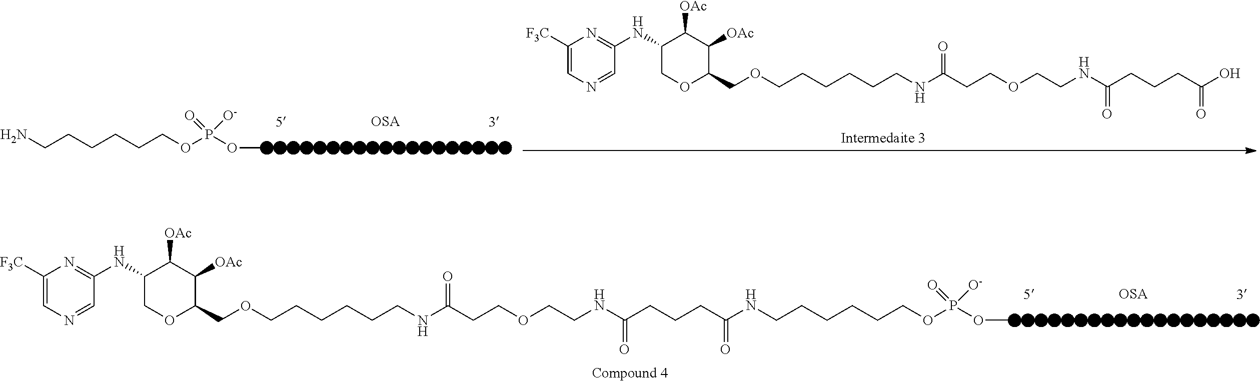 [SMILES: CC(=O)O[C@@H]1[C@H](OC(C)=O)[C@@H](Nc2cncc(C(F)(F)F)n2)CO[C@@H]1COCCCCCCNC(=O)CCOCCNC(=O)CCCC(=O)O ; COP(=O)([O-])OCCCCCCN ; COP(=O)([O-])OCCCCCCNC(=O)CCCC(=O)NCCOCCC(=O)NCCCCCCOC[C@H]1OC[C@H](Nc2cncc(C(F)(F)F)n2)[C@@H](OC(C)=O)[C@H]1OC(C)=O]

To a solution of Intermediate 3 acid (15 mg, 5 eq.) in DMF (200 ul) was added TFA-O—PFP (70 uL 64 eq.) and followed by DIPEA (75 uL, 64 eq.) at 25° C. The tube was shaken for 1 h at 25° C.

The reaction was quenched with H₂O. The resulting mixture was diluted with DMF (800 ul), followed by addition of the oligo-amine solution (50 mg in 1 mL 10×PBS, pH 7.4). The tube was shaken at 25° C. for 16 h; the reaction mixture was treated by 28% NH₄₀H (2 mL) for 5 h, 81.14% DP by LCMS.

Purified by IP—RP-HPLC, obtained TEA salt: 29 mg, 98.14% purity.

### Purification Condition:

Waters Xbridge BEH C18 10 μm, 19*250 mm;
Mobile Phase A: 100 mM HFIP-5 mM TEA in water
Mobile Phase B: 80% ACN; Column temperature: 45° C.

Converted to sodium salt: The TEA salt was dissolved in 1.5 mL (1M, NaOAc) at rt. for 5 min, diluted with EtOH (15 mL) and cooled to −40° C. for 0.5 h. The resulting precipitate was centrifuged, and the collected solid was washed with EtOH (10 mL*2), dissolved in Milli Q. water, and lyophilized to give Compound 4 sodium salt: 27 mg (by OD), 100% purity.

### LCMS Condition:

Instrument: LCMS—Agilent 1260 Prime-G6230
Column: XBridge® Prmier Oligo BEH C18 130 Å 2.5 m, 2.1*100 mm
Mobile Phase A: 100 mM HFIP-5 mM TEA in water
Mobile Phase B: 80% ACN; Column temperature: 45 oC.
Ion Mode: ESI Negative

TABLE 10
Oligonucleotide synthesis conditions

| Compound ID | Calcd.MW | Observed MW |
|---|---|---|
| Compound 4 | 7915.47 | 7915.57 |

### Table 11. Representative Compounds

TABLE 11
Representative Compounds

| # | Structure |
|---|---|
| 1 | 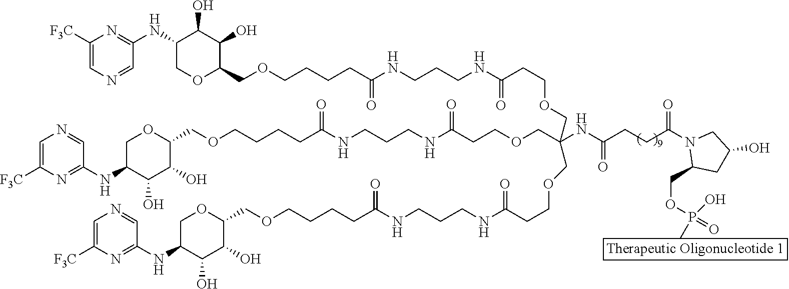 |
| 2 | 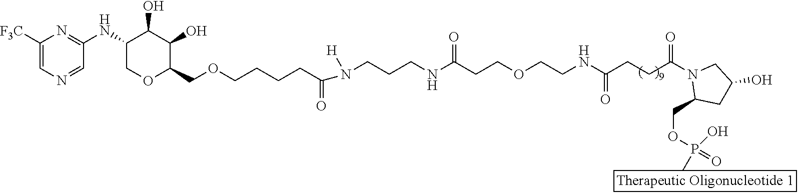 |
| 3 | 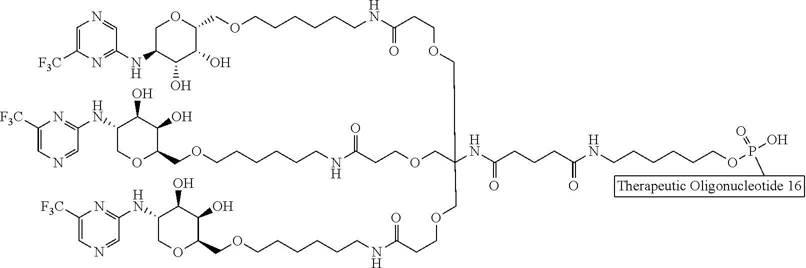 [SMILES: CP(=O)(O)OCCCCCCNC(=O)CCCC(=O)NC(COCCC(=O)NCCCCCCOC[C@H]1OC[C@H](Nc2cncc(C(F)(F)F)n2)[C@@H](O)[C@H]1O)(COCCC(=O)NCCCCCCOC[C@H]1OC[C@H](Nc2cncc(C(F)(F)F)n2)[C@@H](O)[C@H]1O)COCCC(=O)NCCCCCCOC[C@H]1OC[C@H](Nc2cncc(C(F)(F)F)n2)[C@@H](O)[C@H]1O] |
| 4 | 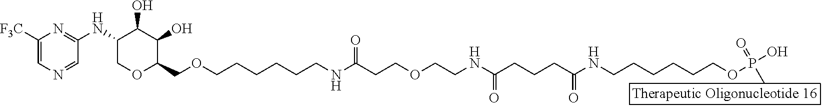 [SMILES: CP(=O)(O)OCCCCCCNC(=O)CCCC(=O)NCCOCCC(=O)NCCCCCCOC[C@H]1OC[C@H](Nc2cncc(C(F)(F)F)n2)[C@@H](O)[C@H]1O] |
| 5 | 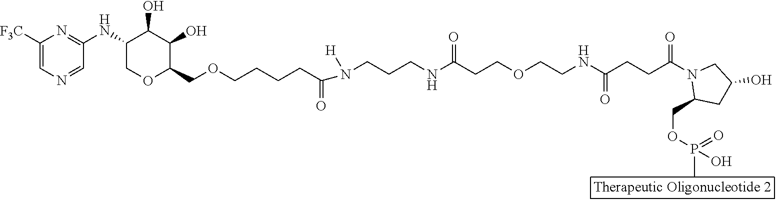 [SMILES: CP(=O)(O)OC[C@@H]1C[C@@H](O)CN1C(=O)CCC(=O)NCCOCCC(=O)NCCCNC(=O)CCCCOC[C@H]1OC[C@H](Nc2cncc(C(F)(F)F)n2)[C@@H](O)[C@H]1O] |
| 6 | 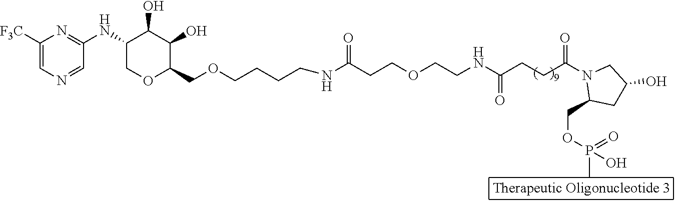 |
| 7 | 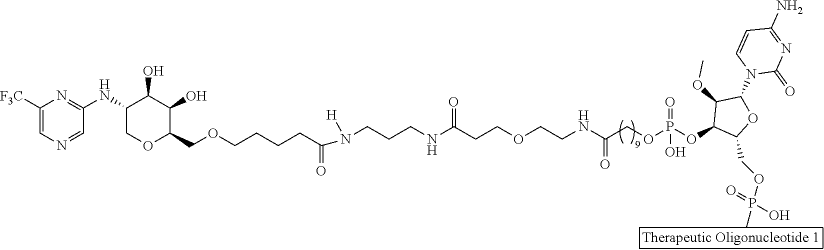 |
| 8 | 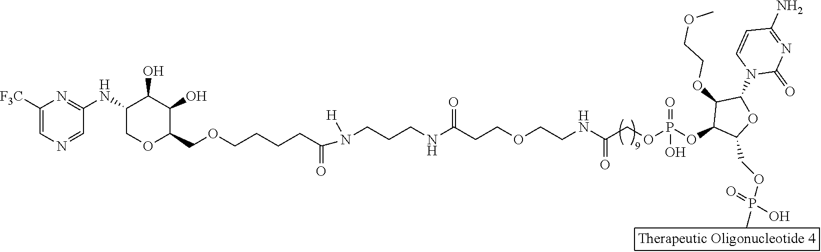 |
| 9 | 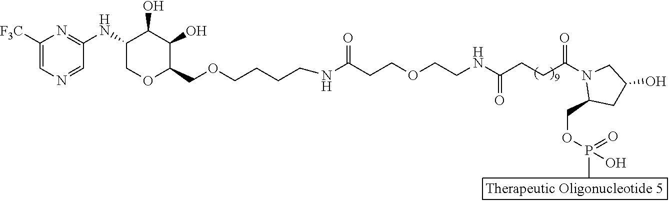 |
| 10 | 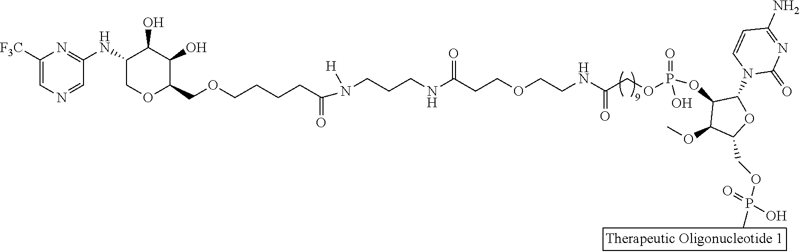 |
| 11 | 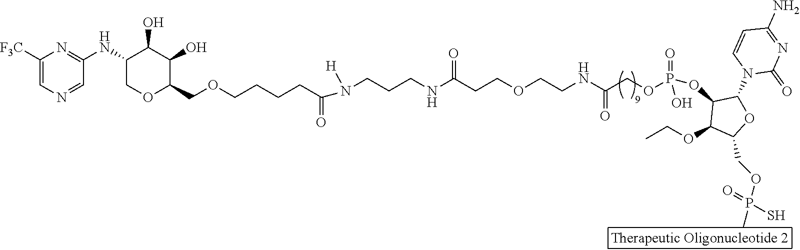 |

### Example 6. Synthesis of Givosiran

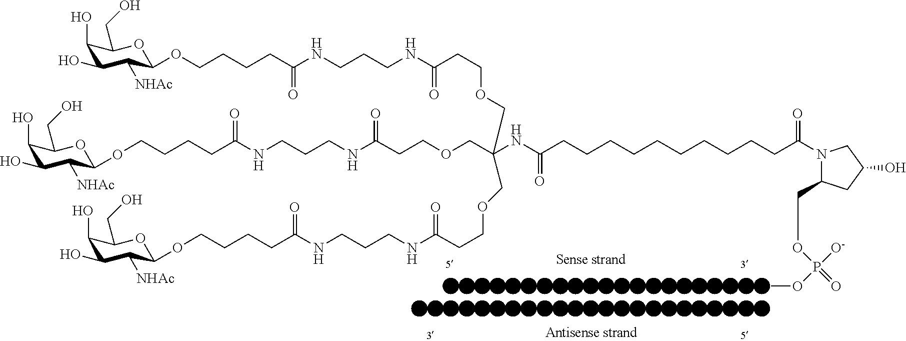

Sense Strand:
(SEQ ID NO: 11)
5′- c*a*gaaaGaGuGuCuCaucuua-3′-[L96 GalNAc]
Antisense Strand:
(SEQ ID NO: 12)
5′-u*A*AGaUgAgAcAcUcUuUcUg*g*u-3′
Abbreviation:
Lower case (e.g. a/u/g/c) = 2′-OMe;
upper case (e.g. A/U/G/C) = 2′-F;
* = phosphorothioate linkage (PS bond)

### Step 1. Synthesis of the Sense Strand (SS) and Antisense Strand (AS)

TABLE 12
Oligonucleotide synthesis conditions

| | |
|---|---|
| Synthesizer | MerMade 12 |
| CPG | SS: L96-GalNAc CPG (500 A) |
| | AS: Universal CPG (500A, 90 umol/g) |
| Scale (umol) | SS: 50 umol; |
| | AS: 50 umol |
| Coupling | 50 umol, 0.1 M amidite, 5 eq. each time; |
| | 0.25 M ETT (5 mL each time); 2 times, |
| | 12 min in total. |
| Oxidation | 0.02 M iodine solution in MeCN/pyridine/H₂O |
| Sulfurization | ADTT (0.2 M, 80 eq.) in pyridine, 7 min |
| Cleavage and | 20% DEA (50 mL), 60 mins; 28% |
| deprotection (C & D) | NH4OH/EtOH = 3:1, 45° C., 20 h. |
| Result | After HPLC purification, obtained: |
| | SS: purified by IP-RP-HPLC, |
| | obtained TEA salt: 90.2 mg, 100% purity |
| | AS: purified by IP-RP-HPLC, |
| | obtained TEA salt: 63.76 mg, 100% purity; |
| | converted to Na salt, obtained Na salt: |
| | SS: 83.51 mg (by OD), 99.4% purity |
| | AS: 61.8 mg (by OD), 98.79% purity |

### Step 2. Formation of Duplex

To form siRNA duplex, equal molar amounts of AS and SS was mixed then heated to 95° C. for 2 minutes. The resulting mixture was lyophilized to give final siRNA duplex.

Obtained siRNA duplex: 102 mg (by OD), 97.40% purity
SS calculated molecular weight: 8736.55
SS observed molecular weight: 8736.37
AS calculated molecular weight: 7563.87
AS observed molecular weight: 7563.89

### Example 7. Target Gene Silencing

### Instrumentation

QuantStudio 7 Flex Fast Real-Time PCR system (Applied biosystem) and Countstar Rigel S2 (Countstar)

### Materials Used

Primary human hepatocytes (PHHs) cells (lot: BXU) are maintained in InvitroGRO CP Medium (BioreclamationIVT-S03316) supplemented with 10% FBS (ExCell Bio-FSP500), and 1% Penicillin Streptomycin Solution (HyClone-SV30010).

The key reagents are AceQ Universal U+ Probe Master Mix V2 (Vazyme-Q513-03), RNeasy 96 Kit (12) (Qiagen-74182), HiScript III RT SuperMix for qPCR (Vazyme-R³²³-01), TaqMan Gene Expression Assay (GAPDH, Thermo), TaqMan Gene Expression Assay (ALAS1, Thermo).

### Assay Protocol:

### Seeding PHH Cells and Adding Compound at the Same Time

First, the test siRNA was diluted with Nuclease-free water to make 10× final concentration (e.g. the final test concentration was 150 nM, make it as 1500 nM), then, siRNA is further 1:4 diluted with water to get 12 points. Cryo-PHIs were thawed in 37° C. water bath, and transferred to prewarmed InvitroGRO CP Medium, and mixed slowly. The cell density was measured by Countstar Rigel S2, then the final cell density was adjusted to 6×10⁵ cell/ml. The diluted siRNA series were transferred to 96-well assay plates, followed by addition of cell suspension. The cell plates were placed in incubator at 37° C., 5% CO₂ for 24, or 48 hours.

### RNA Extraction and Reverse Transcription

The medium was removed and the cells were used for RNA purification. The intracellular RNA was isolated with RNeasy kit (Qiagen-74182) according to the Manual. Reverse transcription by using HiScript III RT SuperMix for qPCR (Vazyme-R³²³-01).

qPCR

The PCR was performed with 95° C. for 10 min, then cycling at 95° C. for 15 sec, 60° C. for 1 min for 40 cycles.

TABLE 13
q-PCR conditions

| Component | Volume (μl, 1×reaction) |
|---|---|
| AceQ Universal U+ Probe Master Mix V2 | 5 |
| GAPDH gene TaqMan® Gene Expression | 0.17 |
| Assays (60×)/target gene TaqMan® Gene | |
| Expression Assays (60×) | |
| cDNA | 2 |
| Nuclease-free water | Up to total 10ul |

### Data Analysis

The expression of the target gene in each test sample is determined by relative quantitation (RQ) using the comparative Ct (ΔΔCt) method.

$$Δ CT = average Ct of target gene - average Ct of GAPDH$$

$$Δ Δ CT = Δ CT ( sample ) - Δ CT ( untreated cell ) ;$$

$$Relative quantity of target gene mRNA = {2}^{- Δ Δ CT}$$

$$\% inhibition = ( relative quantity of untreated cell - relative quantity of sample ) / relative quantity of untreated cell \times 100 \%$$

EC₅₀ and EC₉₀ values (log(agonist) vs. response—Variable slope) will be calculated with GraphPad Prism software.

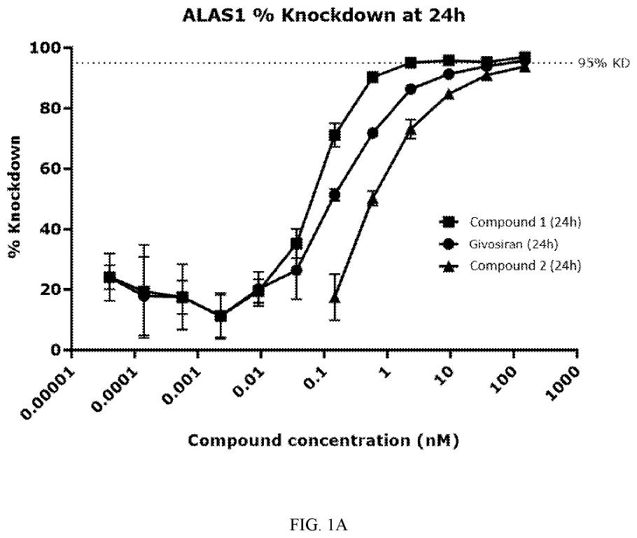

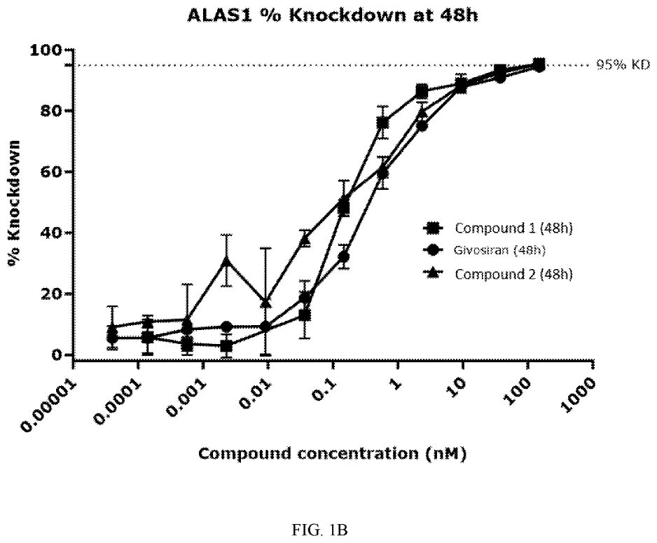

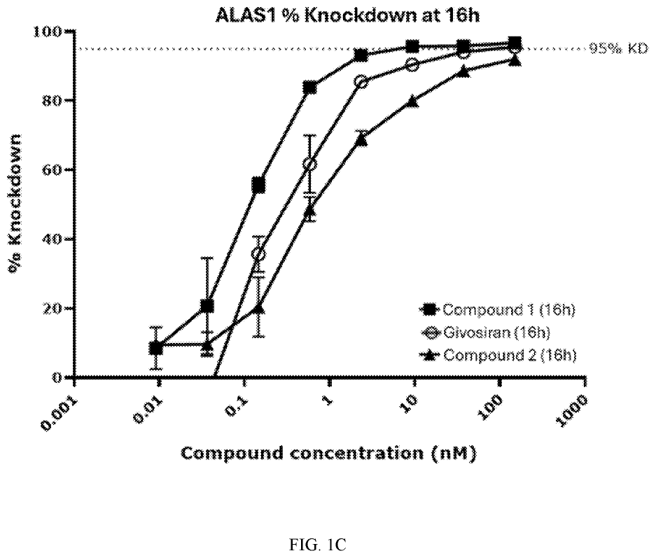

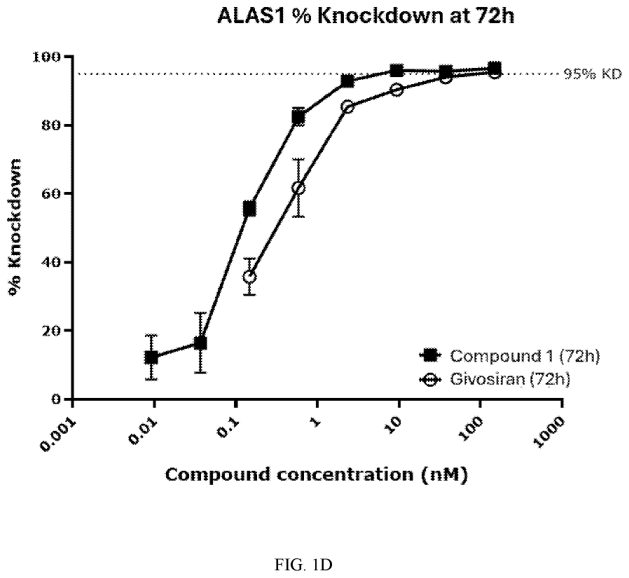

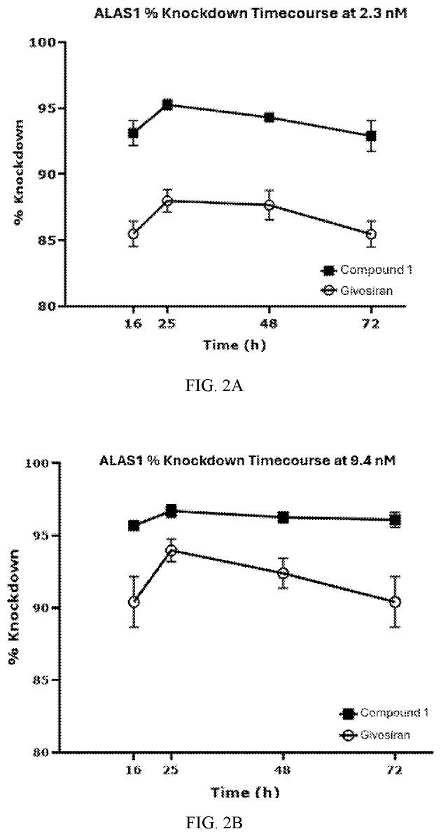

The resulting data is provided in FIGS. 1A, 1B, 1C, 1D, 2A, and 2B.

TABLE 14
siRNA silencing of target gene expression in primary human hepatocytes

| Incubation | | Absolute | Relative | Highest | |
|---|---|---|---|---|---|
| time | Compound | EC₅₀ | EC₉₀ | Inhibition | Unit |
| 16 hrs | Compound 1 | 0.08 | | 96.21 | nM |
| 16 hrs | Compound 2 | 0.69 | | 92.30 | |
| 16 hrs | Givosiran | 0.25 | | 93.94 | |
| 24 hrs | Compound 1 | *** | | ** | nM |
| 24 hrs | Compound 2 | *** | | | |
| 24 hrs | Givosiran | *** | ** | ** | |
| 48 hrs | Compound 1 | * | * | | nM |
| 48 hrs | Compound 2 | * | | | |
| 48 hrs | Givosiran | | | | |
| 72 hrs | Compound 1 | 0.13 | | 97.02 | nM |
| 72 hrs | Givosiran | 0.31 | | 95.69 | |

<10 nM = ***; >10 nM and <100 nM = **; >100 nM = *

### Example 8. Compound 1 Pharmacokinetics in Rats for IV Bolus and SC Formulation

### Dose FORMULATIONS

The formulations were prepared on the day of use.

### For IV Bolus Formulation:

An appropriate amount of test article was dissolved to vehicle, stirred to obtain a clear solution by visual inspection. The dosing solution was sterile-filtered through a 0.22 μM filter before dosing.

For SC formulation:

An appropriate amount of compounds was dissolved to vehicle, stirred to obtain a clear solution or homogenous suspension by visual inspection.

### Blood Collection and Plasma

Following IV bolus or subcutaneous administration of compound 1 to male SD rats, blood was collected (about 0.2 mL per time point) from jugular vein of each animal. All blood samples were transferred into pre-cooled low binding EP tubes containing 4 uL of K2-EDTA (0.5 M) as anti-coagulant placed on wet ice. After blood was collected, the samples were processed for plasma by centrifugation at approximately 4° C., 3200 g for 10 minutes, and the plasma was obtained within 30 minutes of blood collection. The plasma was transferred to another clean low binding EP tube and quick frozen over dry ice and kept −60° C. or lower until LC-MS/MS analysis.

### Pharmacokinetics Data Analysis

Plasma concentration versus time data was plotted in graph and analyzed by non-compartmental approaches using the Phoenix WinNonlin software program (version 6.3 or above, Pharsight, Mountain View, CA). Related PK parameters were calculated according to dosing route, e.g. Cl, Vdss and C₀ for intravenous administration, Cₘₐₓ, Tₘₐₓ or % F for extravascular administration, and T_{1/2}, AUC₍₀₋ₜ₎, AUC_{(0-inf)}, MRT₍₀₋ₜ₎, MRT_{(0-inf)} for all routes.

TABLE 15
Calculated PK parameters in
rats for IV bolus and SC formulation

| | Compound 1 | |
|---|---|---|
| PK parameters | IV 5 mg/kg | SC 5 mg/kg |
| Cₘₐₓ (μg/mL) | NA | 0.322 |
| Tₘₐₓ (h) | NA | 0.833 |
| AUC_{inf} (h * μg/mL) | 6.67 | 0.953 |
| CI (mL/min/kg) | 12.8 | Na |
| Vdₛₛ (L/kg) | 0.0544 | NA |
| T_{1/2} (h) | 0.0959 | 1.82 |
| % F | NA | 14.3 |

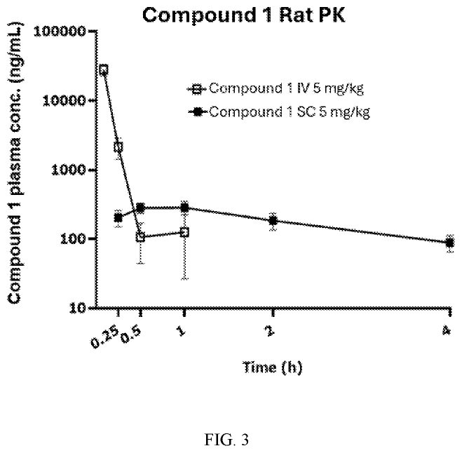

The rat PK data is provided in FIG. 3.

### Example 9. a Liver Distribution Study of Givosiran and Compound 1 in Female SD Rats Administered Via Subcutaneous Injection

### Test Articles

$$Compound 1 - M . W . = 16564.3 g / mol , F . W . = 17509.74 amu$$

$$Givosiran - M . W . = 16300.42 g / mol , F . W . = 17245.63 amu$$

Both compounds were stored at −20° C.

### Dose Formulations

The formulations were prepared on the day of use.

### For SC Formulation:

An appropriate amount of compounds were dissolved to vehicle, and stirred to obtain a clear solution or homogenous suspension by visual inspection.

Formulation Sample Collection Formulation samples were removed from each of the formulation solutions or suspensions, transferred into 1.5 mL of polypropylene microcentrifuge tubes and run dose validation by LC/UV or LC-MS/MS.

### Animal Specifications

45 Female SD rats of the age of about 8 weeks and body weight below 250 g. All animals were fed.

### Study Design

TABLE 16
The experimental design

| | No. of | | | | | | |
|---|---|---|---|---|---|---|---|
| | SD Rats | Treatment | | | | | |
| | (#/ | | | | Dose | | |
| Group | gender) | Test | Dose | Conc. | Volume | | Dose |
| # | Female | Article | (mg/kg) | (mg/mL) | (mL/kg) | Vehicle | Route |
| 1 | 5 | vehicle | — | — | 10 | Saline | SC |
| 2 | 5 | Givosiran | 1 | 0.1 | 10 | Saline | SC |
| 3 | 5 | Givosiran | 2.5 | 0.25 | 10 | Saline | SC |
| 4 | 5 | Givosiran | 5 | 0.5 | 10 | Saline | SC |
| 5 | 5 | Givosiran | 10 | 1 | 10 | Saline | SC |
| 6 | 5 | Compound 1 | 1 | 0.1 | 10 | Saline | SC |
| 7 | 5 | Compound 1 | 2.5 | 0.25 | 10 | Saline | SC |
| 8 | 5 | Compound 1 | 5 | 0.5 | 10 | Saline | SC |
| 9 | 5 | Compound 1 | 10 | 1 | 10 | Saline | SC |

### Dose Administration:

Animals were weighed prior to dose administration and dose volume were determined based on body weights.

### Sample Collection and Preparation

TABLE 17
The schedule for sample collection

| | Animal No. | |
|---|---|---|
| Group | Male | Sampling Time points (min) |
| 1 | R01, R02, R03, | Liver: 72 hr |
| | R04, R05 (Fed) | Analytes: ALAS1 mRNA level |
| 2 | R06, R07, R08, | Liver: 72 hr |
| | R09, R10 (Fed) | Analytes: ALAS1 mRNA |
| | | level and Givosiran |
| | | (antisense (AS) chain) |
| 3 | R11, R12, R13, | Liver: 72 hr |
| | R14, R15 (Fed) | Analytes: ALAS1 mRNA |
| | | level and Givosiran |
| | | (antisense (AS) chain) |
| 4 | R16, R17, R18, | Liver: 72 hr |
| | R19, R20 (Fed) | Analytes: ALAS1 mRNA |
| | | level and Givosiran |
| | | (antisense (AS) chain) |
| 5 | R21, R22, R23, | Liver: 72 hr |
| | R24, R25 (Fed) | Analytes: ALAS1 mRNA |
| | | level and Givosiran |
| | | (antisense (AS) chain) |
| 6 | R26, R27, R28, | Liver: 72 hr |
| | R29, R30 (Fed) | Analytes: ALAS1 mRNA |
| | | level and Compound 1 |
| | | (antisense (AS) chain) |
| 7 | R31, R32, R33, | Liver: 72 hr |
| | R34, R35 (Fed) | Analytes: ALAS1 mRNA |
| | | level and Compound 1 |
| | | (antisense (AS) chain) |
| 8 | R36, R37, R38, | Liver: 72 hr |
| | R39, R40 (Fed) | Analytes: ALAS1 mRNA |
| | | level and Compound 1 |
| | | (antisense (AS) chain) |
| 9 | R41, R42, R43, | Liver: 72 hr |
| | R44, R45 (Fed) | Analytes: ALAS1 mRNA |
| | | level and Compound 1 |
| | | (antisense (AS) chain) |

### Tissue Collection:

After animals were euthanized, their livers were harvested.

ALAS1 mRNA analysis: The liver tissues were collected from two different sites on the left half of liver with about 20-50 mg each site. Sampling collection site was kept consistent across all animals in study.

Each tissue was cut to <0.5 cm length and immersed in 1 mL of RNAlater reagent. After they were stored at 4° C. overnight (to allow the solution to thoroughly penetrate the tissue), RNAlater were removed. The tissue samples were kept at −60° C. or lower until homogenization.

Two tissue samples were homogenized separately on wet ice using homogenizing buffer at the ratio of 1:20 (1 g tissue with 20 mL buffer, the homogenizing buffer is RNAiso). Tissue homogenates were transferred to two pre-cooled and labelled low-binding tubes (RNase free) and kept at −60° C. or lower until sample analysis separately. Two samples were pooled before analysis.

PK: Two liver samples (0.1 g×2), one for backup, one for BA) were collected from left half of liver. Sample collection site was kept near one site of PD sample collection, and consistent across all animals in study. About 0.1 g liver was homogenized with homogenizing buffer (Lysis-loading buffer (Phenomenex, clarity OTX)) at the ratio of 1:9 (1 g tissue with 9 mL buffer). About 800 L tissue homogenates were transferred to a pre-cooled and labelled low-binding tube (RNase free) and kept at −60° C. or lower immediately until LC-MS/MS analysis or other use.

### Pharmacokinetics Data Analysis

Plasma concentration versus time data were plotted in graph and analyzed by non-compartmental approaches using the Phoenix WinNonlin software program (version 6.3 or above, Pharsight, Mountain View, CA). Related PK parameters were calculated according to dosing route, e.g. Cl, Vdss and C₀ for intravenous administration, Cₘₐₓ, Tₘₐₓ or % F for extravascular administration, and T_{1/2}, AUC₍₀₋ₜ₎, AUC_{(0-inf)}, MRT₍₀₋ₜ₎, MRT_{(0-inf)} for all routes.

ALAS1 mRNA Analysis

### RNA Extraction

Tissue samples (50 μL) were transferred into round-bottom RNase-free tube. Using RNase-free pipette tips, 300 μL Lysis Buffer was added, mixed well and 70% ethanol (400 μL) was added to the tissue homogenate samples. 50 μL TRIzol was used.

Up to 700 μL of sample was vortexed and transferred to the spin cartridge (with the collection tube) and incubated at room temperature for about 2 minutes. The sample was centrifuged at 12000×g for 30 seconds at room temperature. The flow-through was discarded and 650 μL Wash Buffer I was added to the spin cartridge and the step was repeated. 500 μL wash Buffer II with ethanol was added and the step was repeated. The sample was centrifuged at 12000×g for 2 minutes to dry the membrane with bound RNA, and uncapped for 5 minutes at room temperature for ethanol evaporation. 70 μL RNase-free water was added and the sample was incubated at room temperature for 2 minutes. The sample was centrifuged at 12000×g for 2 minutes at room temperature to elute RNA from the membrane into the recovery tube. RNA was stored at −60° C. until ready for qPCR.

### Reverse Transcription PCR

### Genomic DNA Elimination Reaction

5 μL RNA samples (total quantity: 1000 ng) were added into the PCR 8-strip tubes and then 5 μL of DNA eraser master mix (5× gDNA Eraser Buffer, gDNA Eraser, RNase Free dH2O) was dispensed, and centrifuged briefly to bring the PCR reaction mix to the bottom of the tube. The PCR 8-strip tubes were kept at 42° C. for 2 min, then kept at 4° C.

### Reverse-Transcription Reaction

L of reverse transcription reaction master mix (5× PrimeScript Buffer 2, PrimeScript RT Enzyme Mix I, RT Primer Mix, RNase Free dH2O) was added to the reaction solution from the above step and mixed gently. The mixture was centrifuged briefly to bring the PCR reaction mix to the bottom of the tube and was proceeded immediately with the reverse-transcription reaction by incubating samples at 37° C. for 15 minutes and inactivate the enzyme at 85° C. for 5 seconds. The reverse transcription products were stored below −60° C. until ready for qPCR.

TaqMan Probe Real Time Quantitative PCR (TaqMan qPCR)
cDNA from above step was diluted and the following components were combined in the quantities shown, multiplied by the number of reactions required.

TABLE 18
qPCR reaction components

| | Total reaction | Volume per |
|---|---|---|
| | Components | reaction (uL) |
| TaqMan | 2X Fast Advanced Master Mix | 10.0 |
| RT-qPCR | 20X Target gene FAM TaqMan | |
| (cDNA loading | Gene Expression Assay | 1.0 |
| volume of 4.0 μL) | 20X Reference gene VIC TaqMan | 1.0 |
| | Gene Expression Assay | |
| | RNase-free Water | 4.0 |

16.0 μL of Taqman reaction mixture was dispensed to 384-well reaction plates, to which 2.0 μL of the diluted RNA was added according to plate layout. The 384-well plate were sealed with optical adhesive covers and centrifuged briefly to bring the PCR reaction mix to the bottom of the tube.

TABLE 19
Thermal protocol

| | Step | Temperature | Time | Cycles |
|---|---|---|---|---|
| RT-qPCR | UNG enzyme | 50° C. | 2 mins | 1 |
| thermal | incubation | | | |
| protocol | Polymerase | 95° C. | 30 seconds | 1 |
| | activation | | | |
| | Denaturation | 95° C. | 5 seconds | 40 |
| | Annealing & | 60° C. | 35 seconds | |
| | extension | | | |

The plate was loaded into the real-time PCR instrument and the run was started.

ALAS1 in all samples was normalized to an internal reference gene (Gapdh) detected simultaneously (Multi-plexed) during the TaqMan reaction (CtAlas1)−CtReference(Gapdh)=ΔCt). The relative quantitation or change in gene expression elicited by each test compound is expressed in relation to the vehicle control sample (ΔCtcompound−ΔCtvehicle=ΔΔCt) using the Comparative Method. Fold changes in gene expression are determined by taking 2 to the power of this value (2-ΔΔCt).

TABLE 20
ALAS1 mRNA suppression at 72 hours

| Dose level | ALAS1 mRNA % Knockdown | |
|---|---|---|
| (mg/kg) | Givosiran | Compound 1 |
| 1 | 40.4 + 12.7 | 39.3 + 11.6 |
| 2.5 | 49.7 + 14.2 | 61.1 + 1.9 |
| 5 | 58.4 + 14.3 | 73.3 + 8.0 |
| 10 | 75.9 + 5.0 | 84.5 + 5.5 |

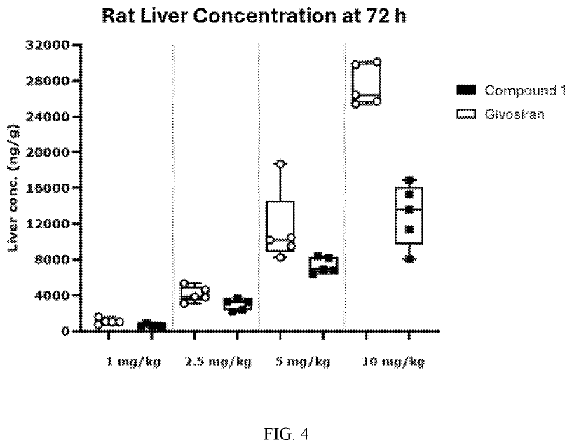

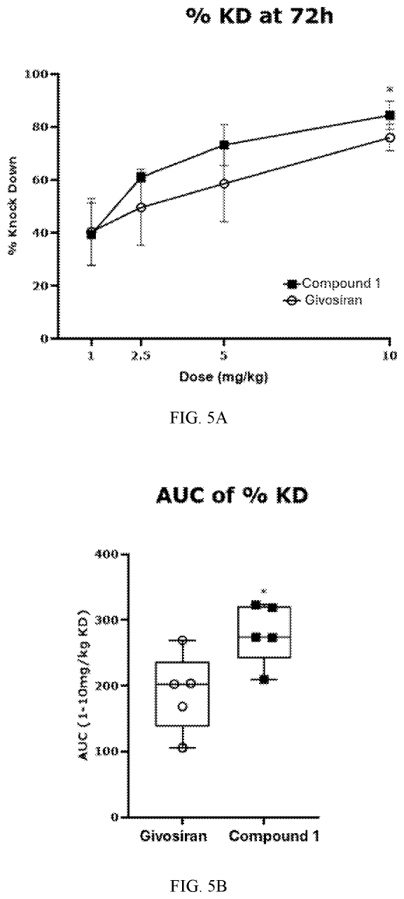

The data for this experiment is provided in FIG. 4, FIG. 5A and FIG. 5B.

All publications and patent applications cited in this specification are herein incorporated by reference as if each individual publication or patent application were specifically and individually indicated to be incorporated by reference.

Although the foregoing invention has been described in some detail by way of illustration and example for the purpose of clarity of understanding, it will be readily apparent to one of ordinary skill in the art in light of the teaching of this invention that certain changes or modifications may be made thereto without departing from the spirit or scope of the invention.

Additionally, those skilled in the art will recognize, or be able to ascertain using no more than routine experimentation, many equivalents to the specific embodiments and methods described herein. Such equivalents are intended to be encompassed by the scope of the present application.

We claim:

1. A compound of Formula (III): 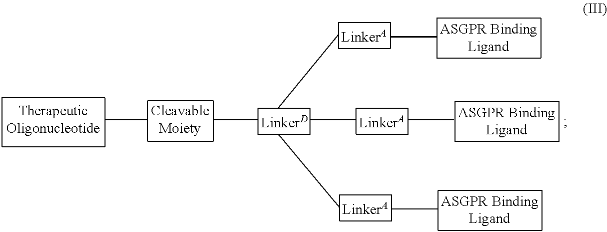
or a pharmaceutically acceptable salt thereof,
wherein:
ASGPR Binding Ligand is a compound selected from: 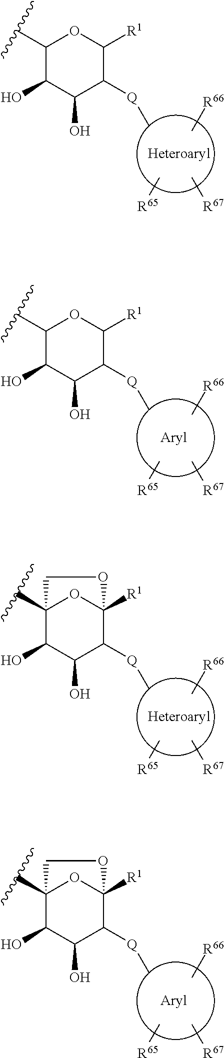 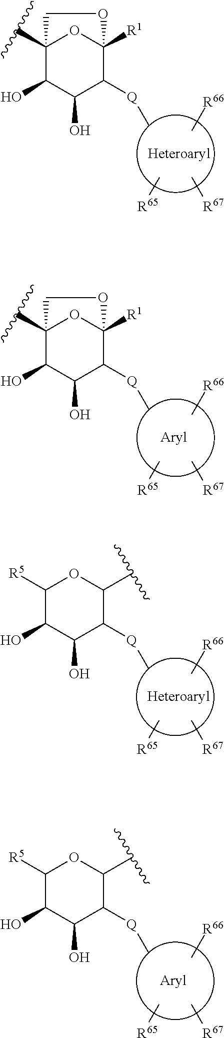 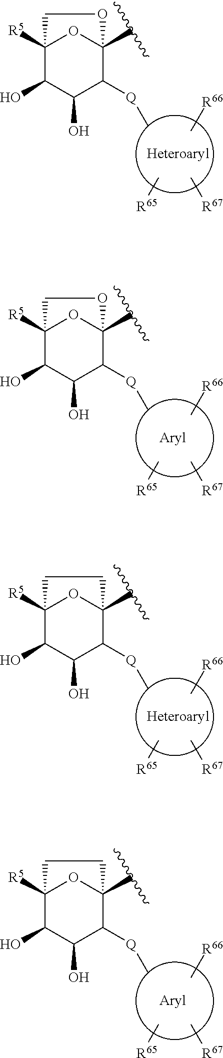
Q is —O— or —N(R¹⁰)—;
R¹ and R⁵ are independently selected from hydrogen, alkyl, alkenyl, alkynyl, haloalkyl, halogen, aryl, arylalkyl, heteroaryl, heteroarylalkyl, heterocycle, heterocycloalkyl, haloalkoxy, C₀-C₆alkyl-cyano, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R′)—C(O)R³, C₀-C₆alkyl-N(R′)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, C₀-C₆alkyl-N═S(O)(R³)₂, C₀-C₆alkylN₃, and C₀-C₆alkyl-O—S(O)₂R³;
R³ at each occurrence is independently selected from the group consisting of hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from the group consisting of hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is hydrogen, alkyl, haloalkyl, arylalkyl, heteroarylalkyl, alkenyl, alkynyl, aryl, heteroaryl, heterocycle, C(O)R³, S(O)R³, C(S)R³, or S(O)₂R³;
R⁶⁵, R⁶⁶, and R⁶⁷ are independently selected at each occurrence from the group consisting of hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, and C₀-C₆alkyl-O—S(O)₂R³, each of which is optionally substituted with 1, 2, or 3 substituents independently selected from R⁸⁰;
R⁸⁰ is independently selected at each instance from the group consisting of alkyl, alkenyl, alkynyl, haloalkyl, —OR⁶, F, Cl, Br, I, —NR⁶R⁷, heterocycle, heteroaryl, aryl, cyano, nitro, hydroxyl, azide, amide, —SR⁶, —S(O)(NR⁶)R³, —NR⁸C(O)R³, —C(O)NR⁶R⁷, and —C(O)R³;
Cleavable Moiety is a bond, 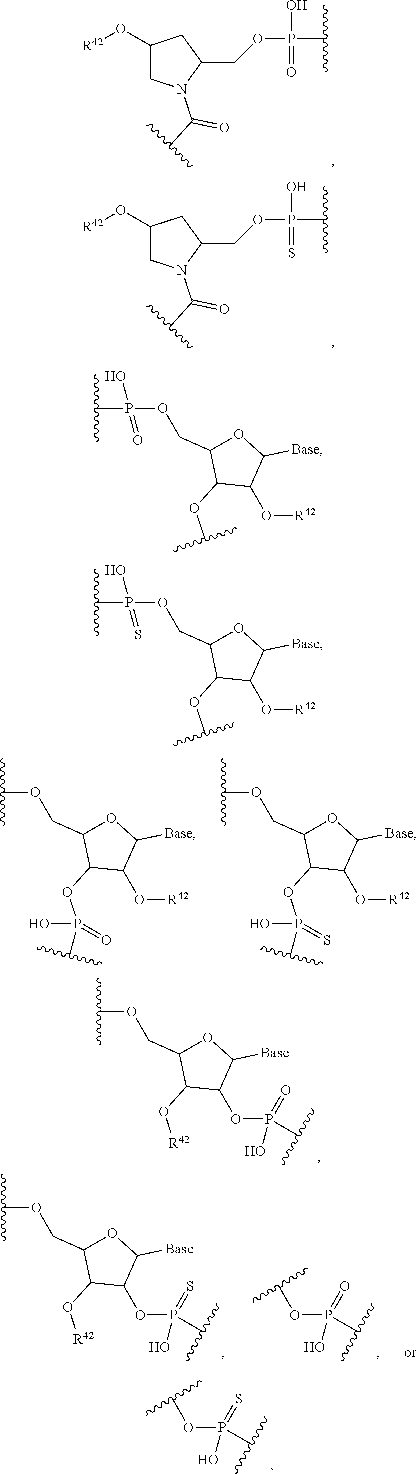
wherein the Therapeutic Oligonucleotide is attached to the —P—, or
Cleavable Moiety is 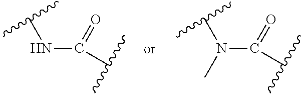
or wherein the Therapeutic Oligonucleotide is bound to the —C— or —N—, or
Cleavable Moiety is 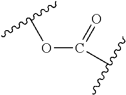 [SMILES: *OC(*)=O]
wherein the Therapeutic Oligonucleotide is bound to the —C— or —O—;
R⁴² is hydrogen, alkyl, alkyl-O-alkyl, or C(O)R⁴³;
R⁴³ is selected from the group consisting of hydrogen, alkyl, and haloalkyl;
Base is a heterocycle comprising two, three, or four nitrogen atoms, wherein the heterocycle is substituted with an oxo, amino, or carbamoyl and optionally substituted with 1, 2, or 3 substituents independently selected from oxo, alkyl, haloalkyl, alkenyl, haloalkenyl, and halogen;
Therapeutic Oligonucleotide is an antisense oligonucleotide, siRNA, shRNA, DNA aptamer, RNA aptamer, miRNA, miRNA mimic, antimiRNA, DNA decoy, RNA decoy, or CpG oligonucleotide;
Linker^{A} is: 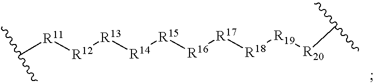
Linker^{D} is: 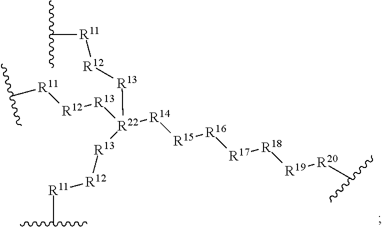
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, —[C(O)—CH₂—NR⁶]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, and 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹; and
X⁻ is an anionic group.

2. The compound of claim 1 wherein:
Cleavable Moiety is a bond, 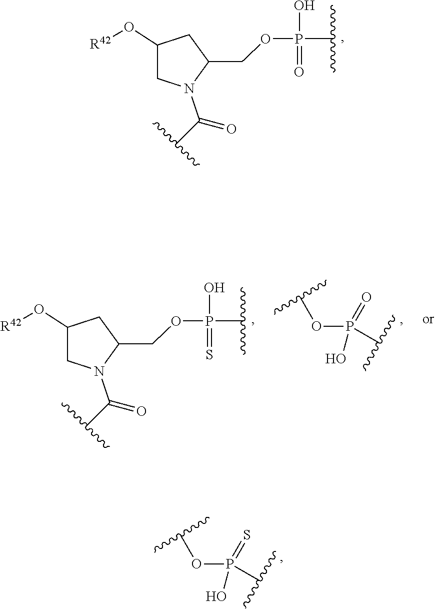
wherein the Therapeutic Oligonucleotide is attached to the —P—;
R⁴² is hydrogen, alkyl, alkyl-O-alkyl, or C(O)R⁴³; and
R⁴³ is selected from the group consisting of hydrogen, alkyl, and haloalkyl.

3. The compound of claim 1, wherein R¹ is hydrogen.

4. The compound of claim 1, wherein the ASGPR Binding Ligand is selected from the group consisting of:

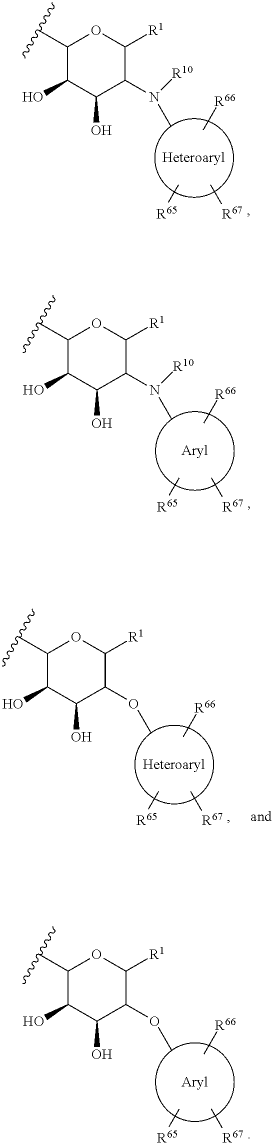

5. The compound of claim 1, wherein the ASGPR Binding Ligand is

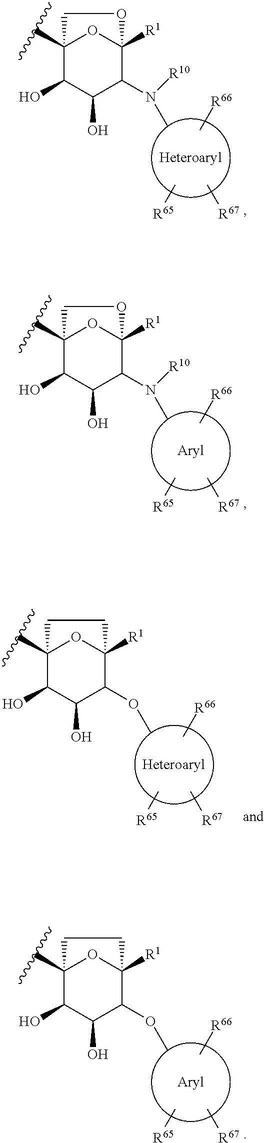

6. The compound of claim 4, wherein the ASGPR Binding Ligand is

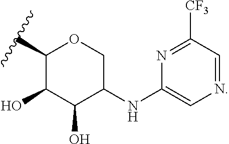 [SMILES: *[C@H]1OCC(Nc2cncc(C(F)(F)F)n2)[C@@H](O)[C@H]1O]

7. The compound of claim 4, wherein the ASGPR Binding Ligand is

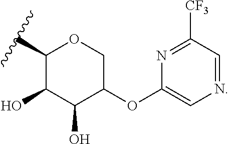 [SMILES: *[C@H]1OCC(Oc2cncc(C(F)(F)F)n2)[C@@H](O)[C@H]1O]

8. The compound of claim 4, wherein the ASGPR Binding Ligand is

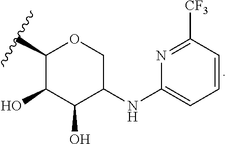 [SMILES: *[C@H]1OCC(Nc2cccc(C(F)(F)F)n2)[C@@H](O)[C@H]1O]

9. The compound of claim 4, wherein the ASGPR Binding Ligand is

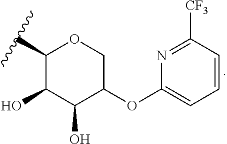 [SMILES: *[C@H]1OCC(Oc2cccc(C(F)(F)F)n2)[C@@H](O)[C@H]1O]

10. The compound of claim 4, wherein the ASGPR Binding Ligand is

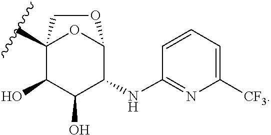 [SMILES: *[C@@]12CO[C@@H](O1)[C@H](Nc1cccc(C(F)(F)F)n1)[C@@H](O)[C@H]2O]

11. The compound of claim 4, wherein the ASGPR Binding Ligand is

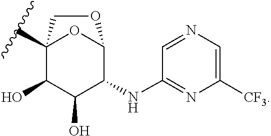 [SMILES: *[C@@]12CO[C@@H](O1)[C@H](Nc1cncc(C(F)(F)F)n1)[C@@H](O)[C@H]2O]

12. The compound of claim 1, wherein Cleavable Moiety is 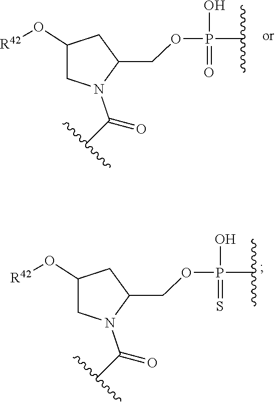
wherein the Therapeutic Oligonucleotide is attached to the —P—.

13. The compound of claim 12, wherein R⁴² is methyl.

14. The compound of claim 12, wherein R⁴² is hydrogen.

15. The compound of claim 1, wherein the compound is: 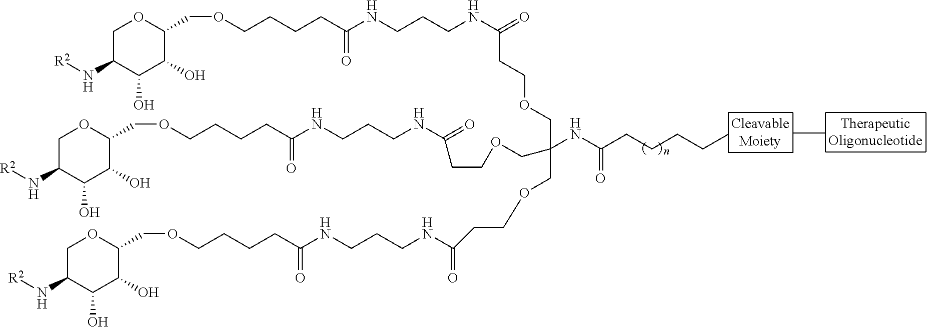
or a pharmaceutically acceptable salt thereof;
wherein
n is 0, 1, 2, 3, 4, 5, 6, 7, 8, 9, or 10; and
R² is

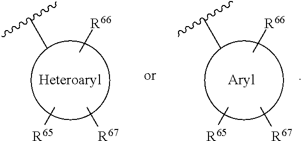

16. The compound of claim 1, wherein the compound is selected from the group consisting of: 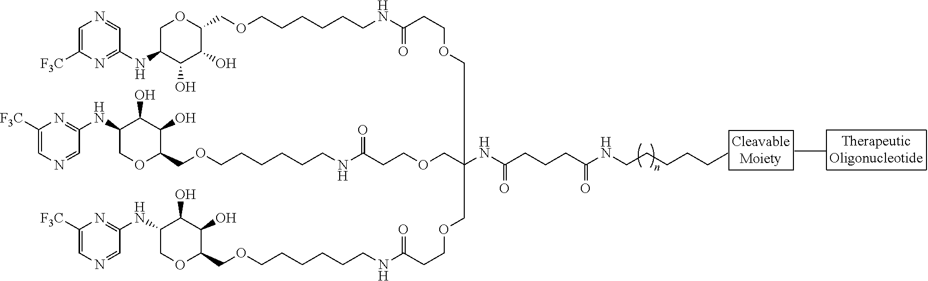
or a pharmaceutically acceptable salt thereof,
wherein
n is 0, 1, 2, 3, 4, 5, 6, 7, 8, 9, or 10.

17. The compound of claim 1, wherein Therapeutic Oligonucleotide is a nucleotide that is at least 95% identical to a nucleotide described in Table 1.

18. The compound of claim 1, wherein the Therapeutic Oligonucleotide is Therapeutic Oligonucleotide 1.

19. The compound of claim 1, wherein the Therapeutic Oligonucleotide is Therapeutic Oligonucleotide 16.

20. A compound selected from 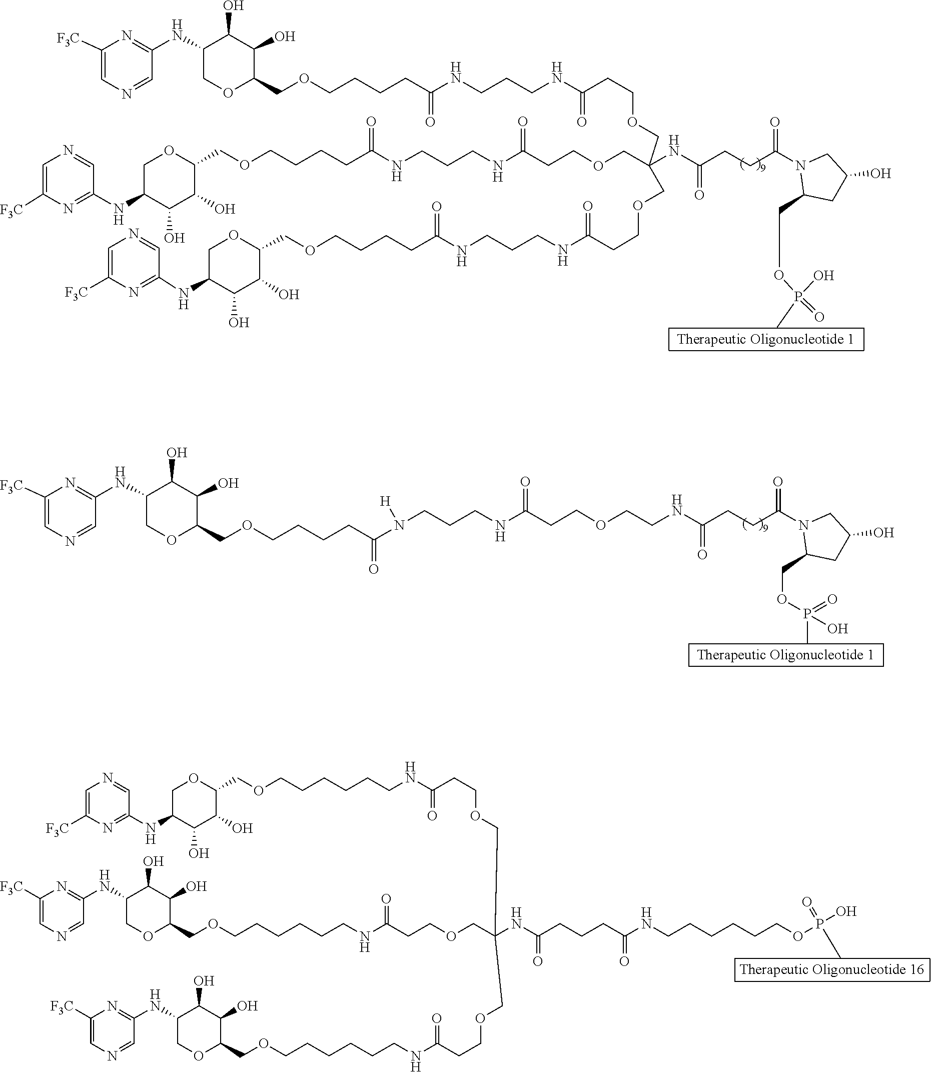 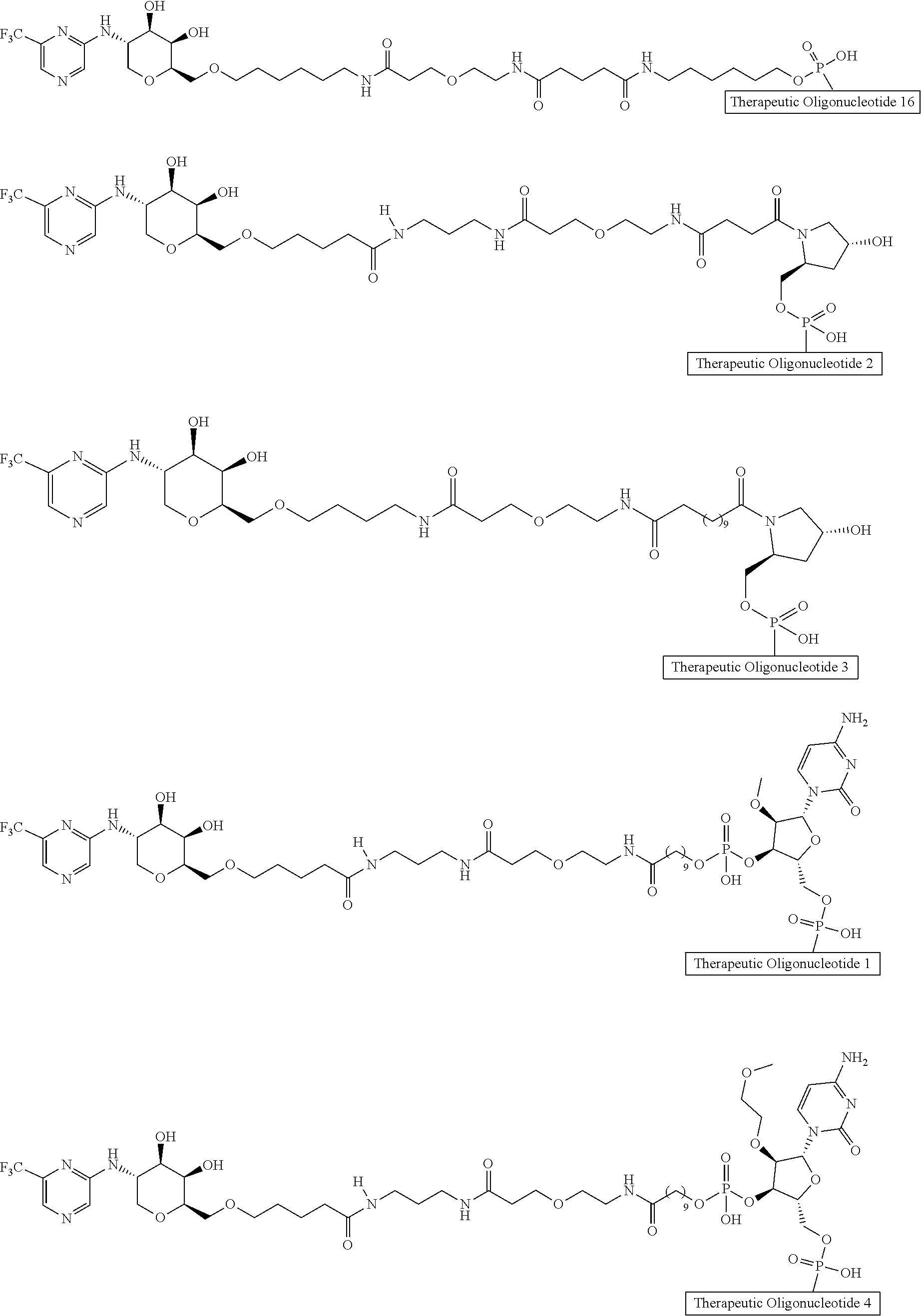 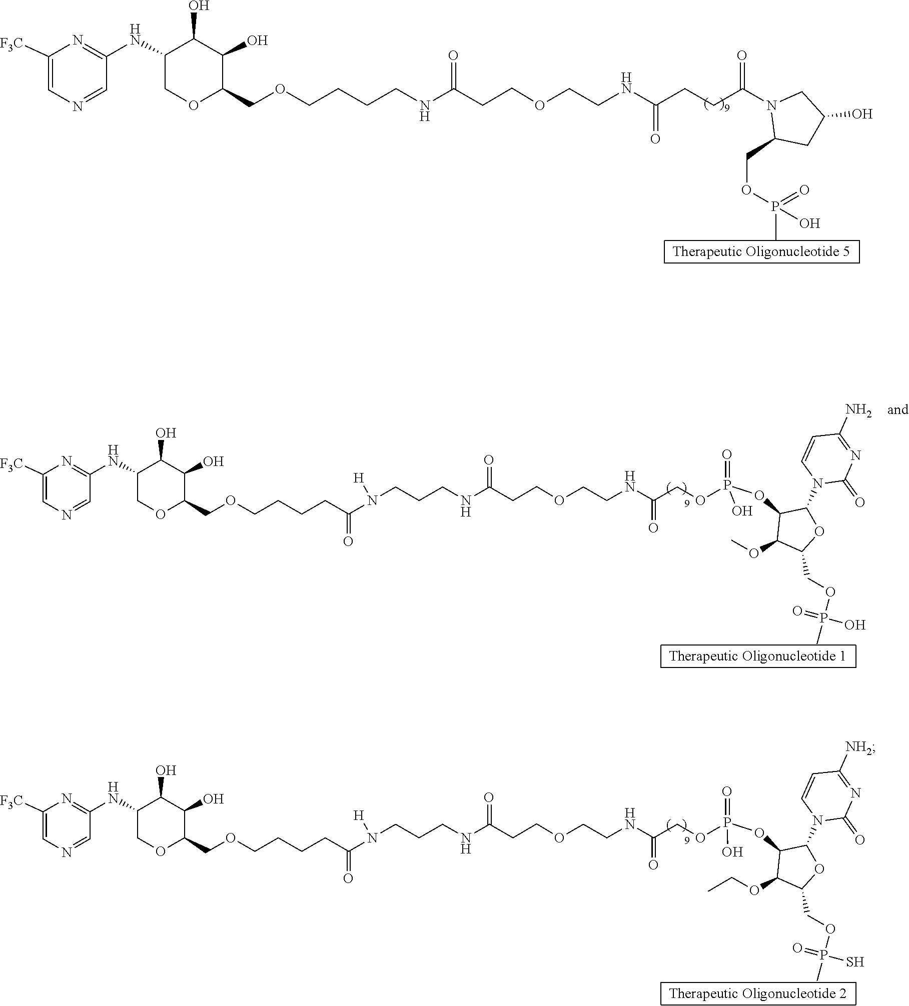
or a pharmaceutically acceptable salt thereof.

21. A pharmaceutical composition comprising a compound of claim 1 or a pharmaceutically acceptable salt thereof and a pharmaceutically acceptable excipient.


SEQUENCE LISTING (62 sequences)

SEQ ID NO: 1 - protein, 14 aa, synthetic construct
SPSTPPTPSPSTPP

SEQ ID NO: 2 - protein, 20 aa, synthetic construct
SPSTPPTPSPSTPPSPSTPP

SEQ ID NO: 3 - protein, 14 aa, synthetic construct
SPTSPPSPTPTSPP

SEQ ID NO: 4 - protein, 14 aa, synthetic construct
PPTSPSPTPPTSPS

SEQ ID NO: 5 - protein, 16 aa, synthetic construct
PPTSPSPTPPTSPSPP

SEQ ID NO: 6 - protein, 14 aa, synthetic construct
SPPSTPSPTPTSPP

SEQ ID NO: 7 - protein, 14 aa, synthetic construct
SPSTPSTPSPSTPP

SEQ ID NO: 8 - protein, 20 aa, synthetic construct
PPTSPSPTPPTSPSSPSTPP

SEQ ID NO: 9 - protein, 14 aa, synthetic construct
PPSTPTPSPPSTPS

SEQ ID NO: 10 - protein, 16 aa, synthetic construct
PPSTPTPSPPSTPSPP

SEQ ID NO: 11 - RNA, 21 nt, synthetic construct
  source 1..21: mol_type = other RNA
  modified_base 1: mod_base = cm
  modified_base 2: mod_base = OTHER; note = 2'-O-methyladenosine
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-deoxy-2'-fluoro-guanine
  modified_base 8: mod_base = OTHER; note = 2'-O-methyladenosine
  modified_base 9: mod_base = OTHER; note = 2'-deoxy-2'-fluoro-guanine
  modified_base 10: mod_base = um
  modified_base 11: mod_base = OTHER; note = 2'-deoxy-2'-fluoro-guanine
  modified_base 12: mod_base = um
  modified_base 13: mod_base = OTHER; note = 2'-deoxy-2'-fluoro-cytosine
  modified_base 14: mod_base = um
  modified_base 15: mod_base = OTHER; note = 2'-deoxy-2'-fluoro-cytosine
  modified_base 16: mod_base = OTHER; note = 2'-O-methyladenosine
  modified_base 17: mod_base = um
  modified_base 18: mod_base = cm
  modified_base 19: mod_base = um
  modified_base 20: mod_base = um
  modified_base 21: mod_base = OTHER; note = 2'-O-methyladenosine
cagaaagagtgtctcatctta

SEQ ID NO: 12 - RNA, 23 nt, synthetic construct
  source 1..23: mol_type = other RNA
  modified_base 1: mod_base = um
  modified_base 2: mod_base = OTHER; note = 2'-deoxy-2'-fluoro-adenosine
  modified_base 3: mod_base = OTHER; note = 2'-deoxy-2'-fluoro-adenosine
  modified_base 4: mod_base = OTHER; note = 2'-deoxy-2'-fluoro-guanine
  modified_base 5: mod_base = OTHER; note = 2'-O-methyladenosine
  modified_base 6: mod_base = OTHER; note = 2'-deoxy-2'-fluoro-uridine
  modified_base 7: mod_base = gm
  modified_base 8: mod_base = OTHER; note = 2'-deoxy-2'-fluoro-adenosine
  modified_base 9: mod_base = gm
  modified_base 10: mod_base = OTHER; note = 2'-deoxy-2'-fluoro-adenosine
  modified_base 11: mod_base = cm
  modified_base 12: mod_base = OTHER; note = 2'-deoxy-2'-fluoro-adenosine
  modified_base 13: mod_base = cm
  modified_base 14: mod_base = OTHER; note = 2'-deoxy-2'-fluoro-uridine
  modified_base 15: mod_base = cm
  modified_base 16: mod_base = OTHER; note = 2'-deoxy-2'-fluoro-uridine
  modified_base 17: mod_base = um
  modified_base 18: mod_base = OTHER; note = 2'-deoxy-2'-fluoro-uridine
  modified_base 19: mod_base = cm
  modified_base 20: mod_base = OTHER; note = 2'-deoxy-2'-fluoro-uridine
  modified_base 21: mod_base = gm
  modified_base 22: mod_base = gm
  modified_base 23: mod_base = um
taagatgagacactctttctggt

SEQ ID NO: 13 - RNA, 21 nt, synthetic construct
  source 1..21: mol_type = other RNA
  modified_base 1: mod_base = gm
  modified_base 2: mod_base = OTHER; note = 2'-O-methyladenosine
  modified_base 3: mod_base = cm
  modified_base 4: mod_base = um
  modified_base 5: mod_base = um
  modified_base 6: mod_base = um
  modified_base 7: mod_base = OTHER; note = 2'-deoxy-2'-fluoro-cytosine
  modified_base 8: mod_base = OTHER; note = 2'-O-methyladenosine
  modified_base 9: mod_base = OTHER; note = 2'-deoxy-2'-fluoro-uridine
  modified_base 10: mod_base = OTHER; note = 2'-deoxy-2'-fluoro-cytosine
  modified_base 11: mod_base = OTHER; note = 2'-deoxy-2'-fluoro-cytosine
  modified_base 12: mod_base = um
  modified_base 13: mod_base = gm
  modified_base 14: mod_base = gm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = um
  modified_base 19: mod_base = OTHER; note = 2'-O-methyladenosine
  modified_base 20: mod_base = um
  modified_base 21: mod_base = OTHER; note = 2'-O-methyladenosine
gactttcatcctggaaatata

SEQ ID NO: 14 - RNA, 23 nt, synthetic construct
  source 1..23: mol_type = other RNA
  modified_base 1: mod_base = um
  modified_base 2: mod_base = OTHER; note = 2'-deoxy-2'-fluoro-adenosine
  modified_base 3: mod_base = um
  modified_base 4: mod_base = OTHER; note = 2'-O-methyladenosine
  modified_base 5: mod_base = um
  modified_base 6: mod_base = OTHER; note = 2'-deoxy-2'-fluoro-uridine
  modified_base 7: mod_base = um
  modified_base 8: mod_base = OTHER; note = 2'-deoxy-2'-fluoro-cytosine
  modified_base 9: mod_base = OTHER; note = 2'-deoxy-2'-fluoro-cytosine
  modified_base 10: mod_base = OTHER; note = 2'-O-methyladenosine
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-deoxy-2'-fluoro-uridine
  modified_base 15: mod_base = gm
  modified_base 16: mod_base = OTHER; note = 2'-deoxy-2'-fluoro-adenosine
  modified_base 17: mod_base = OTHER; note = 2'-O-methyladenosine
  modified_base 18: mod_base = OTHER; note = 2'-O-methyladenosine
  modified_base 19: mod_base = gm
  modified_base 20: mod_base = um
  modified_base 21: mod_base = cm
  modified_base 22: mod_base = cm
  modified_base 23: mod_base = OTHER; note = 2'-O-methyladenosine
tatatttccaggatgaaagtcca

SEQ ID NO: 15 - RNA, 23 nt, synthetic construct
  source 1..23: mol_type = other RNA
  modified_base 1: mod_base = OTHER; note = 2'-O-methyladenosine
  modified_base 2: mod_base = OTHER; note = 2'-deoxy-2'-fluoro-cytosine
  modified_base 3: mod_base = OTHER; note = 2'-O-methyladenosine
  modified_base 4: mod_base = OTHER; note = 2'-deoxy-2'-fluoro-adenosine
  modified_base 5: mod_base = OTHER; note = 2'-deoxy-2'-fluoro-adenosine
  modified_base 6: mod_base = OTHER; note = 2'-deoxy-2'-fluoro-adenosine
  modified_base 7: mod_base = gm
  modified_base 8: mod_base = OTHER; note = 2'-deoxy-2'-fluoro-cytosine
  modified_base 9: mod_base = OTHER; note = 2'-O-methyladenosine
  modified_base 10: mod_base = OTHER; note = 2'-deoxy-2'-fluoro-adenosine
  modified_base 11: mod_base = OTHER; note = 2'-O-methyladenosine
  modified_base 12: mod_base = OTHER; note = 2'-deoxy-2'-fluoro-adenosine
  modified_base 13: mod_base = cm
  modified_base 14: mod_base = OTHER; note = 2'-deoxy-2'-fluoro-adenosine
  modified_base 15: mod_base = gm
  modified_base 16: mod_base = OTHER; note = 2'-deoxy-2'-fluoro-guanine
  modified_base 17: mod_base = um
  modified_base 18: mod_base = OTHER; note = 2'-deoxy-2'-fluoro-cytosine
  modified_base 19: mod_base = um
  modified_base 20: mod_base = OTHER; note = 2'-O-methyladenosine
  modified_base 21: mod_base = gm
  modified_base 22: mod_base = OTHER; note = 2'-O-methyladenosine
  modified_base 23: mod_base = OTHER; note = 2'-O-methyladenosine
acaaaagcaaaacaggtctagaa

SEQ ID NO: 16 - RNA, 21 nt, synthetic construct
  source 1..21: mol_type = other RNA
  modified_base 1: mod_base = cm
  modified_base 2: mod_base = um
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-deoxy-2'-fluoro-cytosine
  modified_base 8: mod_base = um
  modified_base 9: mod_base = OTHER; note = 2'-deoxy-2'-fluoro-guanine
  modified_base 10: mod_base = um
  modified_base 11: mod_base = OTHER; note = 2'-deoxythymidine-3'-phosphate
  modified_base 12: mod_base = um
  modified_base 13: mod_base = um
  modified_base 14: mod_base = gm
  modified_base 15: mod_base = cm
  modified_base 16: mod_base = um
  modified_base 17: mod_base = um
  modified_base 18: mod_base = um
  modified_base 19: mod_base = um
  modified_base 20: mod_base = gm
  modified_base 21: mod_base = um
ctagacctgttttgcttttgt

SEQ ID NO: 17 - RNA, 21 nt, synthetic construct
  source 1..21: mol_type = other RNA
  modified_base 1: mod_base = OTHER; note = 2'-deoxy-2'-fluoro-guanine
  modified_base 2: mod_base = gm
  modified_base 3: mod_base = OTHER; note = 2'-deoxy-2'-fluoro-uridine
  modified_base 4: mod_base = um
  modified_base 5: mod_base = OTHER; note = 2'-deoxy-2'-fluoro-adenosine
  modified_base 6: mod_base = OTHER; note = 2'-O-methyladenosine
  modified_base 7: mod_base = OTHER; note = 2'-deoxy-2'-fluoro-cytosine
  modified_base 8: mod_base = OTHER; note = 2'-O-methyladenosine
  modified_base 9: mod_base = OTHER; note = 2'-deoxy-2'-fluoro-cytosine
  modified_base 10: mod_base = OTHER; note = 2'-deoxy-2'-fluoro-cytosine
  modified_base 11: mod_base = OTHER; note = 2'-deoxy-2'-fluoro-adenosine
  modified_base 12: mod_base = um
  modified_base 13: mod_base = OTHER; note = 2'-deoxy-2'-fluoro-uridine
  modified_base 14: mod_base = um
  modified_base 15: mod_base = OTHER; note = 2'-deoxy-2'-fluoro-adenosine
  modified_base 16: mod_base = cm
  modified_base 17: mod_base = OTHER; note = 2'-deoxy-2'-fluoro-uridine
  modified_base 18: mod_base = um
  modified_base 19: mod_base = OTHER; note = 2'-deoxy-2'-fluoro-cytosine
  modified_base 20: mod_base = OTHER; note = 2'-O-methyladenosine
  modified_base 21: mod_base = OTHER; note = 2'-deoxy-2'-fluoro-adenosine
ggttaacaccatttacttcaa

SEQ ID NO: 18 - RNA, 23 nt, synthetic construct
  source 1..23: mol_type = other RNA
  modified_base 1: mod_base = um
  modified_base 2: mod_base = OTHER; note = 2'-deoxy-2'-fluoro-uridine
  modified_base 3: mod_base = gm
  modified_base 4: mod_base = OTHER; note = 2'-deoxy-2'-fluoro-adenosine
  modified_base 5: mod_base = OTHER; note = 2'-O-methyladenosine
  modified_base 6: mod_base = OTHER; note = 2'-deoxy-2'-fluoro-guanine
  modified_base 7: mod_base = um
  modified_base 8: mod_base = OTHER; note = 2'-deoxy-2'-fluoro-adenosine
  modified_base 9: mod_base = OTHER; note = 2'-O-methyladenosine
  modified_base 10: mod_base = OTHER; note = 2'-deoxy-2'-fluoro-adenosine
  modified_base 11: mod_base = um
  modified_base 12: mod_base = gm
  modified_base 13: mod_base = gm
  modified_base 14: mod_base = OTHER; note = 2'-deoxy-2'-fluoro-uridine
  modified_base 15: mod_base = gm
  modified_base 16: mod_base = OTHER; note = 2'-deoxy-2'-fluoro-uridine
  modified_base 17: mod_base = um
  modified_base 18: mod_base = OTHER; note = 2'-deoxy-2'-fluoro-adenosine
  modified_base 19: mod_base = OTHER; note = 2'-O-methyladenosine
  modified_base 20: mod_base = OTHER; note = 2'-deoxy-2'-fluoro-cytosine
  modified_base 21: mod_base = cm
  modified_base 22: mod_base = OTHER; note = 2'-O-methyladenosine
  modified_base 23: mod_base = gm
ttgaagtaaatggtgttaaccag

SEQ ID NO: 19 - DNA, 16 bp, synthetic construct
  source 1..16: mol_type = other DNA
  modified_base 1: mod_base = OTHER; note = d([2'-O-(2-methoxyethyl)]-5-methyl-ribocytosine
  modified_base 2: mod_base = OTHER; note = 3'-4'-linked-[2'-O-(2-methoxyethyl)]riboguanine
  modified_base 3: mod_base = OTHER; note = [2',5'-anhydro-6'-deoxy-4'-C-(hydroxymethyl)-alpha- L-mannofurano]-5-methyl-cytosine
  modified_base 12: mod_base = m5c
  modified_base 13: mod_base = m5c
  modified_base 14: mod_base = OTHER; note = 3'-4'-linked-[2',5'-anhydro-6'-deoxy-4'-C-(hydroxym ethyl)-alpha-L-mannofurano]-guanine
  modified_base 15: mod_base = OTHER; note = [2',5'-anhydro-6'-deoxy-4'-C-(hydroxymethyl)-alpha- L-mannofurano]-guanine
  modified_base 16: mod_base = OTHER; note = [2'-O-(2-methoxyethyl)]r-guanine
cgctgatttgtccggg

SEQ ID NO: 20 - RNA, 21 nt, synthetic construct
  source 1..21: mol_type = other RNA
  modified_base 1: mod_base = OTHER; note = 2'-O-methyladenosine
  modified_base 2: mod_base = OTHER; note = 2'-O-methyladenosine
  modified_base 3: mod_base = OTHER; note = OTHER; 2'-deoxy-2'-fluoro-guanine
  modified_base 4: mod_base = cm
  modified_base 5: mod_base = OTHER; note = 2'-deoxy-2'-fluoro-adenosine
  modified_base 6: mod_base = OTHER; note = 2'-O-methyladenosine
  modified_base 7: mod_base = OTHER; note = 2'-deoxy-2'-fluoro-guanine
  modified_base 8: mod_base = OTHER; note = 2'-O-methyladenosine
  modified_base 9: mod_base = OTHER; note = 2'-deoxy-2'-fluoro-uridine
  modified_base 10: mod_base = OTHER; note = 2'-deoxy-2'-fluoro-adenosine
  modified_base 11: mod_base = OTHER; note = 2'-deoxy-2'-fluoro-uridine
  modified_base 12: mod_base = um
  modified_base 13: mod_base = OTHER; note = 2'-deoxy-2'-fluoro-uridine
  modified_base 14: mod_base = um
  modified_base 15: mod_base = um
  modified_base 16: mod_base = OTHER; note = 2'-deoxy-2'-fluoro-adenosine
  modified_base 17: mod_base = um
  modified_base 18: mod_base = OTHER; note = 2'-deoxy-2'-fluoro-adenosine
  modified_base 19: mod_base = OTHER; note = 2'-O-methyladenosine
  modified_base 20: mod_base = um
  modified_base 21: mod_base = OTHER; note = 2'-O-methyladenosine
aagcaagatatttttataata

SEQ ID NO: 21 - RNA, 25 nt, synthetic construct
  source 1..25: mol_type = other RNA
  modified_base 1: mod_base = um
  modified_base 2: mod_base = OTHER; note = 2'-deoxy-2'-fluoro-adenosine
  modified_base 3: mod_base = OTHER; note = 2'-deoxy-2'-fluoro-uridine
  modified_base 4: mod_base = um
  modified_base 5: mod_base = OTHER; note = 2'-deoxy-2'-fluoro-adenosine
  modified_base 6: mod_base = um
  modified_base 7: mod_base = OTHER; note = 2'-O-methyladenosine
  modified_base 8: mod_base = OTHER; note = 2'-deoxy-2'-fluoro-adenosine
  modified_base 9: mod_base = OTHER; note = 2'-O-methyladenosine
  modified_base 10: mod_base = OTHER; note = 2'-deoxy-2'-fluoro-adenosine
  modified_base 11: mod_base = OTHER; note = 2'-O-methyladenosine
  modified_base 12: mod_base = um
  modified_base 13: mod_base = OTHER; note = 2'-O-methyladenosine
  modified_base 14: mod_base = OTHER; note = 2'-deoxy-2'-fluoro-uridine
  modified_base 15: mod_base = cm
  modified_base 16: mod_base = OTHER; note = 2'-deoxy-2'-fluoro-uridine
  modified_base 17: mod_base = um
  modified_base 18: mod_base = OTHER; note = 2'-deoxy-2'-fluoro-guanine
  modified_base 19: mod_base = cm
  modified_base 20: mod_base = um
  modified_base 21: mod_base = um
  modified_base 22: mod_base = um
  modified_base 23: mod_base = um
  modified_base 24: mod_base = OTHER; note = 2'-deoxythymidine-3'-phosphate
  modified_base 25: mod_base = OTHER; note = 2'-deoxythymidine-3'-phosphate
tattataaaaatatcttgctttttt

SEQ ID NO: 22 - DNA, 20 bp, synthetic construct
  source 1..20: mol_type = other DNA
  modified_base 1: mod_base = OTHER; note = d([2'-O-(2-methoxyethyl)]-5-methyl-ribouridine
  modified_base 2: mod_base = OTHER; note = [2'-O-(2-methyoxyethyl)]-riboguanine
  modified_base 3: mod_base = OTHER; note = [2'-O-(2-methoxyethyl)]-5-methyl-ribocytidine
  modified_base 4: mod_base = OTHER; note = [2'-O-(2-methoxyethyl)]-riboadenosine
  modified_base 5: mod_base = OTHER; note = [2'-O-(2-methoxyethyl)]-riboadenosine
  modified_base 8: mod_base = m5c
  modified_base 10: mod_base = m5c
  modified_base 15: mod_base = m5c
  modified_base 16: mod_base = OTHER; note = [2'-O-(2-methoxyethyl)]-riboadenosine
  modified_base 17: mod_base = OTHER; note = [2'-O-(2-methoxyethyl)]-riboadenosine
  modified_base 18: mod_base = OTHER; note = [2'-O-(2-methoxyethyl)]-riboadenosine
  modified_base 19: mod_base = OTHER; note = [2'-O-(2-methoxyethyl)]-5-methyl-ribocytidine
  modified_base 20: mod_base = OTHER; note = [2'-O-(2-methoxyethyl)]-riboadenosine
tgcaagtctcttggcaaaca

SEQ ID NO: 23 - RNA, 19 nt, synthetic construct
  source 1..19: mol_type = other RNA
  modified_base 1: mod_base = gm
  modified_base 2: mod_base = um
  modified_base 3: mod_base = gm
  modified_base 4: mod_base = um
  modified_base 5: mod_base = OTHER; note = 2'-deoxy-2'-fluoro-guanine
  modified_base 6: mod_base = cm
  modified_base 7: mod_base = OTHER; note = 2'-deoxy-2'-fluoro-adenosine
  modified_base 8: mod_base = OTHER; note = 2'-deoxy-2'-fluoro-cytosine
  modified_base 9: mod_base = OTHER; note = 2'-deoxy-2'-fluoro-uridine
  modified_base 10: mod_base = um
  modified_base 11: mod_base = cm
  modified_base 12: mod_base = gm
  modified_base 13: mod_base = cm
  modified_base 14: mod_base = um
  modified_base 15: mod_base = um
  modified_base 16: mod_base = cm
  modified_base 17: mod_base = OTHER; note = 2'-O-methyladenosine
  modified_base 18: mod_base = cm
  modified_base 19: mod_base = OTHER; note = 2'-O-methyladenosine
gtgtgcacttcgcttcaca

SEQ ID NO: 24 - RNA, 21 nt, synthetic construct
  source 1..21: mol_type = other RNA
  modified_base 1: mod_base = um
  modified_base 2: mod_base = OTHER; note = 2'-deoxy-2'-fluoro-guanine
  modified_base 3: mod_base = um
  modified_base 4: mod_base = gm
  modified_base 5: mod_base = OTHER; note = 3'-3'-linked-2'-O-methyladenosine
  modified_base 6: mod_base = OTHER; note = 2'-5'-linked-[9-de-beta-D-ribofuranosyl-9-[(2S)-2,3 -dihydroxypropyl]]-adenine
  modified_base 7: mod_base = gm
  modified_base 8: mod_base = OTHER; note = 2'-deoxy-2'-fluoro-cytosine
  modified_base 9: mod_base = OTHER; note = 2'-deoxy-2'-fluoro-guanine
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = um
  modified_base 14: mod_base = OTHER; note = 2'-deoxy-2'-fluoro-guanine
  modified_base 15: mod_base = cm
  modified_base 16: mod_base = OTHER; note = 2'-deoxy-2'-fluoro-adenosine
  modified_base 17: mod_base = cm
  modified_base 18: mod_base = OTHER; note = 2'-O-methyladenosine
  modified_base 19: mod_base = cm
  modified_base 20: mod_base = um
  modified_base 21: mod_base = um
tgtgaagcgaagtgcacactt

SEQ ID NO: 25 - RNA, 21 nt, synthetic construct
  source 1..21: mol_type = other RNA
  modified_base 1: mod_base = gm
  modified_base 2: mod_base = um
  modified_base 3: mod_base = cm
  modified_base 4: mod_base = OTHER; note = 2'-O-methyladenosine
  modified_base 5: mod_base = um
  modified_base 6: mod_base = cm
  modified_base 7: mod_base = OTHER; note = 2'-deoxy-2'-fluoro-cytosine
  modified_base 8: mod_base = OTHER; note = 2'-O-methyladenosine
  modified_base 9: mod_base = OTHER; note = 2'-deoxy-2'-fluoro-cytosine
  modified_base 10: mod_base = OTHER; note = 2'-deoxy-2'-fluoro-adenosine
  modified_base 11: mod_base = OTHER; note = 2'-deoxy-2'-fluoro-adenosine
  modified_base 12: mod_base = um
  modified_base 13: mod_base = gm
  modified_base 14: mod_base = OTHER; note = 2'-O-methyladenosine
  modified_base 15: mod_base = gm
  modified_base 16: mod_base = OTHER; note = 2'-O-methyladenosine
  modified_base 17: mod_base = gm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine
gtcatccacaatgagagtaca

SEQ ID NO: 26 - RNA, 23 nt, synthetic construct
  source 1..23: mol_type = other RNA
  modified_base 1: mod_base = um
  modified_base 2: mod_base = OTHER; note = 2'-deoxy-2'-fluoro-guanine
  modified_base 3: mod_base = um
  modified_base 4: mod_base = OTHER; note = 2'-O-methyladenosine
  modified_base 5: mod_base = OTHER; note = 3'-3'-linked-cm
  modified_base 6: mod_base = OTHER; note = 2'-5'-linked-[1-de-beta-D-ribofuranosyl-1-[(2S)-2,3 -dihydroxypropyl]]-5-methyl-uridine
  modified_base 7: mod_base = cm
  modified_base 8: mod_base = um
  modified_base 9: mod_base = cm
  modified_base 10: mod_base = OTHER; note = 2'-O-methyladenosine
  modified_base 11: mod_base = um
  modified_base 12: mod_base = um
  modified_base 13: mod_base = gm
  modified_base 14: mod_base = OTHER; note = 2'-deoxy-2'-fluoro-uridine
  modified_base 15: mod_base = gm
  modified_base 16: mod_base = OTHER; note = 2'-deoxy-2'-fluoro-guanine
  modified_base 17: mod_base = OTHER; note = 2'-O-methyladenosine
  modified_base 18: mod_base = um
  modified_base 19: mod_base = gm
  modified_base 20: mod_base = OTHER; note = 2'-O-methyladenosine
  modified_base 21: mod_base = cm
  modified_base 22: mod_base = gm
  modified_base 23: mod_base = OTHER; note = 2'-O-methyladenosine
tgtactctcattgtggatgacga

SEQ ID NO: 27 - RNA, 23 nt, synthetic construct
  source 1..23: mol_type = other RNA
  modified_base 1: mod_base = OTHER; note = 2'-O-methyladenosine
  modified_base 2: mod_base = OTHER; note = 2'-deoxy-2'-fluoro-guanine
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = OTHER; note = 3'-3'-linked-gm
  modified_base 7: mod_base = OTHER; note = 2'-5'-[1-de-beta-D-ribofuranosyl-1-[(2S)-2,3-dihydr oxypropyl]]-5-methyl-uridine
  modified_base 8: mod_base = cm
  modified_base 9: mod_base = cm
  modified_base 10: mod_base = OTHER; note = 2'-O-methyladenosine
  modified_base 11: mod_base = um
  modified_base 12: mod_base = gm
  modified_base 13: mod_base = cm
  modified_base 14: mod_base = OTHER; note = 2'-deoxy-2'-fluoro-adenosine
  modified_base 15: mod_base = OTHER; note = 2'-O-methyladenosine
  modified_base 16: mod_base = OTHER; note = 2'-deoxy-2'-fluoro-adenosine
  modified_base 17: mod_base = OTHER; note = 2'-O-methyladenosine
  modified_base 18: mod_base = gm
  modified_base 19: mod_base = cm
  modified_base 20: mod_base = OTHER; note = 2'-O-methyladenosine
  modified_base 21: mod_base = um
  modified_base 22: mod_base = um
  modified_base 23: mod_base = cm
agatagtccatgcaaaagcattc

SEQ ID NO: 28 - RNA, 21 nt, synthetic construct
  source 1..21: mol_type = other RNA
  modified_base 1: mod_base = OTHER; note = 2'-O-methyladenosine
  modified_base 2: mod_base = um
  modified_base 3: mod_base = gm
  modified_base 4: mod_base = cm
  modified_base 5: mod_base = um
  modified_base 6: mod_base = um
  modified_base 7: mod_base = OTHER; note = 2'-deoxy-2'-fluoro-uridine
  modified_base 8: mod_base = um
  modified_base 9: mod_base = OTHER; note = 2'-deoxy-2'-fluoro-guanine
  modified_base 10: mod_base = OTHER; note = 2'-deoxy-2'-fluoro-guanine
  modified_base 11: mod_base = OTHER; note = 2'-deoxy-2'-fluoro-adenosine
  modified_base 12: mod_base = um
  modified_base 13: mod_base = gm
  modified_base 14: mod_base = gm
  modified_base 15: mod_base = OTHER; note = 2'-O-methyladenosine
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = um
atgcttttgcatggactatct

SEQ ID NO: 29 - RNA, 21 nt, synthetic construct
  source 1..21: mol_type = other RNA
  modified_base 1: mod_base = cm
  modified_base 2: mod_base = OTHER; note = 2'-O-methyladenosine
  modified_base 3: mod_base = gm
  modified_base 4: mod_base = cm
  modified_base 5: mod_base = cm
  modified_base 6: mod_base = cm
  modified_base 7: mod_base = cm
  modified_base 8: mod_base = um
  modified_base 9: mod_base = OTHER; note = 2'-deoxy-2'-fluoro-uridine
  modified_base 10: mod_base = OTHER; note = 2'-deoxy-2'-fluoro-adenosine
  modified_base 11: mod_base = OTHER; note = 2'-deoxy-2'-fluoro-uridine
  modified_base 12: mod_base = um
  modified_base 13: mod_base = gm
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = cm
  modified_base 20: mod_base = OTHER; note = 3'-3'-linked-gm
  modified_base 21: mod_base = OTHER; note = 2'-deoxyadenine-3'-phosphate
tcgtataacaataaggggctg

SEQ ID NO: 30 - RNA, 21 nt, synthetic construct
  source 1..21: mol_type = other RNA
  modified_base 1: mod_base = um
  modified_base 2: mod_base = OTHER; note = 2'-deoxy-2'-fluoro-cytosine
  modified_base 3: mod_base = gm
  modified_base 4: mod_base = OTHER; note = 2'-deoxy-2'-fluoro-uridine
  modified_base 5: mod_base = OTHER; note = 2'-O-methyladenosine
  modified_base 6: mod_base = OTHER; note = 2'-deoxy-2'-fluoro-uridine
  modified_base 7: mod_base = OTHER; note = 2'-O-methyladenosine
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-deoxy-2'-fluoro-uridine
  modified_base 13: mod_base = OTHER; note = 2'-O-methyladenosine
  modified_base 14: mod_base = OTHER; note = 2'-deoxy-2'-fluoro-adenosine
  modified_base 15: mod_base = gm
  modified_base 16: mod_base = OTHER; note = 2'-deoxy-2'-fluoro-guanine
  modified_base 17: mod_base = gm
  modified_base 18: mod_base = OTHER; note = 2'-deoxy-2'-fluoro-guanine
  modified_base 19: mod_base = cm
  modified_base 20: mod_base = OTHER; note = 2'-deoxy-2'-fluoro-uridine
  modified_base 21: mod_base = gm
cagccccttattgttatacga

SEQ ID NO: 31 - DNA, 20 bp, synthetic construct
  source 1..20: mol_type = other DNA
  modified_base 1: mod_base = OTHER; note = d([2'-O-(2-methoxyethyl)]-riboadenine
  modified_base 2: mod_base = OTHER; note = [2'-O-(2-methoxyethyl)]-5-methyl-ribocytidine
  modified_base 3: mod_base = OTHER; note = [2'-O-(2-methoxyethyl)]-5-methyl-riboguanine
  modified_base 4: mod_base = OTHER; note = [2'-O-(2-methoxyethyl)]-5-methyl-riboguanine
  modified_base 5: mod_base = OTHER; note = [2'-O-(2-methoxyethyl)]-5-methyl-ribocytidine
  modified_base 13: mod_base = m5c
  modified_base 15: mod_base = m5c
  modified_base 16: mod_base = OTHER; note = d([2'-O-(2-methoxyethyl)]-riboadenine
  modified_base 17: mod_base = OTHER; note = [2'-O-(2-methoxyethyl)]-5-methyl-riboguanine
  modified_base 18: mod_base = OTHER; note = d([2'-O-(2-methoxyethyl)]-5-methyl-ribouridine
  modified_base 19: mod_base = OTHER; note = d([2'-O-(2-methoxyethyl)]-5-methyl-ribouridine
  modified_base 20: mod_base = OTHER; note = d([2'-O-(2-methoxyethyl)]-5-methyl-ribouridine
acggcattggtgcacagttt

SEQ ID NO: 32 - RNA, 36 nt, synthetic construct
  source 1..36: mol_type = other RNA
  modified_base 1: mod_base = OTHER; note = 2'-O-methyladenosine
  modified_base 2: mod_base = um
  modified_base 3: mod_base = OTHER; note = 2'-deoxy-2'-fluoro-guanine
  modified_base 4: mod_base = um
  modified_base 5: mod_base = OTHER; note = 2'-deoxy-2'-fluoro-uridine
  modified_base 6: mod_base = gm
  modified_base 7: mod_base = um
  modified_base 8: mod_base = OTHER; note = 2'-deoxy-2'-fluoro-cytosine
  modified_base 9: mod_base = OTHER; note = 2'-deoxy-2'-fluoro-cytosine
  modified_base 10: mod_base = OTHER; note = 2'-deoxy-2'-fluoro-uridine
  modified_base 11: mod_base = OTHER; note = 2'-deoxy-2'-fluoro-uridine
  modified_base 12: mod_base = um
  modified_base 13: mod_base = OTHER; note = 2'-deoxy-2'-fluoro-uridine
  modified_base 14: mod_base = um
  modified_base 15: mod_base = OTHER; note = 2'-deoxy-2'-fluoro-adenosine
  modified_base 16: mod_base = um
  modified_base 17: mod_base = OTHER; note = 2'-deoxy-2'-fluoro-cytosine
  modified_base 18: mod_base = um
  modified_base 19: mod_base = gm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
atgttgtcctttttatctgagcagccgaaaggctgc

SEQ ID NO: 33 - RNA, 22 nt, synthetic construct
  source 1..22: mol_type = other RNA
  modified_base 1: mod_base = OTHER; note = 4'-5'-linked-([1-de-beta-D-ribofuranosyl-1-[(2R,3R, 4S,5R)-tatrahydro-4-hydroxy-5-[(hydroxymethoxyposphinyl)me thoxy]-3-methoxy-2-furanyl]]-uridine
  modified_base 2: mod_base = OTHER; note = 2'-deoxy-2'-fluoro-cytosine
  modified_base 3: mod_base = OTHER; note = 2'-deoxy-2'-fluoro-adenosine
  modified_base 4: mod_base = OTHER; note = 2'-deoxy-2'-fluoro-guanine
  modified_base 5: mod_base = OTHER; note = 2'-deoxy-2'-fluoro-adenosine
  modified_base 6: mod_base = um
  modified_base 7: mod_base = OTHER; note = 2'-deoxy-2'-fluoro-adenosine
  modified_base 8: mod_base = OTHER; note = 2'-O-methyladenosine
  modified_base 9: mod_base = OTHER; note = 2'-deoxy-2'-fluoro-adenosine
  modified_base 10: mod_base = OTHER; note = 2'-deoxy-2'-fluoro-adenosine
  modified_base 11: mod_base = OTHER; note = 2'-O-methyladenosine
  modified_base 12: mod_base = gm
  modified_base 13: mod_base = gm
  modified_base 14: mod_base = OTHER; note = 2'-deoxy-2'-fluoro-adenosine
  modified_base 15: mod_base = cm
  modified_base 16: mod_base = OTHER; note = 2'-deoxy-2'-fluoro-adenosine
  modified_base 17: mod_base = OTHER; note = 2'-O-methyladenosine
  modified_base 18: mod_base = OTHER; note = 2'-deoxy-2'-fluoro-cytosine
  modified_base 19: mod_base = OTHER; note = 2'-O-methyladenosine
  modified_base 20: mod_base = um
  modified_base 21: mod_base = gm
  modified_base 22: mod_base = gm
tcagataaaaaggacaacatgg

SEQ ID NO: 34 - RNA, 36 nt, synthetic construct
  source 1..36: mol_type = other RNA
  modified_base 1: mod_base = gm
  modified_base 2: mod_base = OTHER; note = 2'-O-methyladenosine
  modified_base 3: mod_base = OTHER; note = 2'-deoxy-2'-fluoro-cytosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-deoxy-2'-fluoro-adenosine
  modified_base 9: mod_base = OTHER; note = 2'-deoxy-2'-fluoro-uridine
  modified_base 10: mod_base = OTHER; note = 2'-deoxy-2'-fluoro-cytosine
  modified_base 11: mod_base = cm
  modified_base 12: mod_base = OTHER; note = 2'-deoxy-2'-fluoro-uridine
  modified_base 13: mod_base = OTHER; note = 2'-deoxy-2'-fluoro-cytosine
  modified_base 14: mod_base = OTHER; note = 2'-O-methyladenosine
  modified_base 15: mod_base = cm
  modified_base 16: mod_base = OTHER; note = 2'-O-methyladenosine
  modified_base 17: mod_base = OTHER; note = 2'-deoxy-2'-fluoro-adenosine
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
gacaaaaatcctcacaataagcagccgaaaggctgc

SEQ ID NO: 35 - RNA, 22 nt, synthetic construct
  source 1..22: mol_type = other RNA
  modified_base 1: mod_base = OTHER; note = ([4'-de(hydroxymethyl)-4'-[(hydroxymethoxyphosphiny l)methoxy]]-methyluridine
  modified_base 2: mod_base = OTHER; note = 2'-deoxy-2'-fluoro-uridine
  modified_base 3: mod_base = OTHER; note = 2'-deoxy-2'-fluoro-adenosine
  modified_base 4: mod_base = um
  modified_base 5: mod_base = OTHER; note = 2'-deoxy-2'-fluoro-uridine
  modified_base 6: mod_base = gm
  modified_base 7: mod_base = OTHER; note = 2'-deoxy-2'-fluoro-uridine
  modified_base 8: mod_base = OTHER; note = 2'-deoxy-2'-fluoro-guanine
  modified_base 9: mod_base = OTHER; note = 2'-O-methyladenosine
  modified_base 10: mod_base = OTHER; note = 2'-deoxy-2'-fluoro-guanine
  modified_base 11: mod_base = gm
  modified_base 12: mod_base = OTHER; note = 2'-deoxy-2'-fluoro-adenosine
  modified_base 13: mod_base = um
  modified_base 14: mod_base = OTHER; note = 2'-deoxy-2'-fluoro-uridine
  modified_base 15: mod_base = um
  modified_base 16: mod_base = OTHER; note = 2'-deoxy-2'-fluoro-uridine
  modified_base 17: mod_base = um
  modified_base 18: mod_base = gm
  modified_base 19: mod_base = OTHER; note = 2'-deoxy-2'-fluoro-uridine
  modified_base 20: mod_base = cm
  modified_base 21: mod_base = gm
  modified_base 22: mod_base = gm
ttattgtgaggatttttgtcgg

SEQ ID NO: 36 - RNA, 19 nt, synthetic construct
  source 1..19: mol_type = other RNA
  modified_base 1: mod_base = OTHER; note = 2'-O-methyladenosine
  modified_base 2: mod_base = OTHER; note = 2'-deoxy-2'-fluoro-uridine
  modified_base 3: mod_base = OTHER; note = 2'-O-methyladenosine
  modified_base 4: mod_base = OTHER; note = 2'-deoxy-2'-fluoro-adenosine
  modified_base 5: mod_base = cm
  modified_base 6: mod_base = OTHER; note = 2'-deoxy-2'-fluoro-uridine
  modified_base 7: mod_base = cm
  modified_base 8: mod_base = OTHER; note = 2'-deoxy-2'-fluoro-uridine
  modified_base 9: mod_base = gm
  modified_base 10: mod_base = OTHER; note = 2'-deoxy-2'-fluoro-uridine
  modified_base 11: mod_base = cm
  modified_base 12: mod_base = OTHER; note = 2'-deoxy-2'-fluoro-cytosine
  modified_base 13: mod_base = OTHER; note = 2'-O-methyladenosine
  modified_base 14: mod_base = OTHER; note = 2'-deoxy-2'-fluoro-uridine
  modified_base 15: mod_base = um
  modified_base 16: mod_base = OTHER; note = 2'-deoxy-2'-fluoro-adenosine
  modified_base 17: mod_base = cm
  modified_base 18: mod_base = OTHER; note = 2'-deoxy-2'-fluoro-cytosine
  modified_base 19: mod_base = gm
cggtaatggacagagttat

SEQ ID NO: 37 - RNA, 19 nt, synthetic construct
  source 1..19: mol_type = other RNA
  modified_base 1: mod_base = cm
  modified_base 2: mod_base = gm
  modified_base 3: mod_base = gm
  modified_base 4: mod_base = um
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-deoxy-2'-fluoro-uridine
  modified_base 8: mod_base = OTHER; note = 2'-deoxy-2'-fluoro-guanine
  modified_base 9: mod_base = OTHER; note = 2'-deoxy-2'-fluoro-guanine
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = gm
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = OTHER; note = 2'-O-methyladenosine
  modified_base 19: mod_base = um
ataactctgtccattaccg

SEQ ID NO: 38 - DNA, 20 bp, synthetic construct
  source 1..20: mol_type = other DNA
  modified_base 1: mod_base = OTHER; note = d([2'-O-(2-methoxyethyl)]-5-methyl-ribouridine
  modified_base 2: mod_base = OTHER; note = [2'-O-(2-methoxyethyl)]-riboguanine
  modified_base 3: mod_base = OTHER; note = [2'-O-(2-methoxyethyl)]-5-methyl-ribocytidine
  modified_base 4: mod_base = OTHER; note = [2'-O-(2-methoxyethyl)]-5-methyl-ribouridine
  modified_base 5: mod_base = OTHER; note = [2'-O-(2-methoxyethyl)]-5-methyl-ribocytidine
  modified_base 6: mod_base = m5c
  modified_base 14: mod_base = m5c
  modified_base 16: mod_base = OTHER; note = [2'-O-(2-methoxyethyl)]-5-methyl-ribouridine
  modified_base 17: mod_base = OTHER; note = [2'-O-(2-methoxyethyl)]-riboguanine
  modified_base 18: mod_base = OTHER; note = [2'-O-(2-methoxyethyl)]-5-methyl-ribouridine
  modified_base 19: mod_base = OTHER; note = [2'-O-(2-methoxyethyl)]-5-methyl-ribouridine
  modified_base 20: mod_base = OTHER; note = [2'-O-(2-methoxyethyl)]-5-methyl-ribocytidine
tgctccgttggtgcttgttc

SEQ ID NO: 39 - DNA, 20 bp, synthetic construct
  source 1..20: mol_type = other DNA
  modified_base 1: mod_base = OTHER; note = d(P-thio)([2'-O-(2-methoxyethyl)]-riboadenine
  modified_base 2: mod_base = OTHER; note = [2'-O-(2-methoxyethyl)]-riboguanine
  modified_base 3: mod_base = OTHER; note = [2'-O-(2-methoxyethyl)]-5-methyl-ribocytidine
  modified_base 4: mod_base = OTHER; note = [2'-O-(2-methoxyethyl)]-5-methyl-ribouridine
  modified_base 5: mod_base = OTHER; note = [2'-O-(2-methoxyethyl)]-5-methyl-ribouridine
  modified_base 6: mod_base = m5c
  modified_base 11: mod_base = m5c
  modified_base 12: mod_base = m5c
  modified_base 15: mod_base = m5c
  modified_base 16: mod_base = OTHER; note = [2'-O-(2-methoxyethyl)]-5-methyl-ribouridine
  modified_base 17: mod_base = OTHER; note = [2'-O-(2-methoxyethyl)]-5-methyl-ribouridine
  modified_base 18: mod_base = OTHER; note = [2'-O-(2-methoxyethyl)]-5-methyl-ribouridine
  modified_base 19: mod_base = OTHER; note = [2'-O-(2-methoxyethyl)]-riboadenine
  modified_base 20: mod_base = OTHER; note = [2'-O-(2-methoxyethyl)]-5-methyl-ribouridine
agcttcttgtccagctttat

SEQ ID NO: 40 - DNA, 20 bp, synthetic construct
  source 1..20: mol_type = other DNA
  modified_base 1: mod_base = OTHER; note = d(P-thio)([2'-O-(2-methoxyethyl)]-riboadenine
  modified_base 2: mod_base = OTHER; note = [2'-O-(2-methoxyethyl)]-5-methyl-ribouridine
  modified_base 3: mod_base = OTHER; note = [2'-O-(2-methoxyethyl)]-5-methyl-ribocytidine
  modified_base 4: mod_base = OTHER; note = [2'-O-(2-methoxyethyl)]-5-methyl-ribocytidine
  modified_base 5: mod_base = OTHER; note = [2'-O-(2-methoxyethyl)]-5-methyl-ribocytidine
  modified_base 7: mod_base = m5c
  modified_base 9: mod_base = m5c
  modified_base 10: mod_base = m5c
  modified_base 11: mod_base = m5c
  modified_base 12: mod_base = m5c
  modified_base 16: mod_base = OTHER; note = [2'-O-(2-methoxyethyl)]-5-methyl-ribocytidine
  modified_base 17: mod_base = OTHER; note = [2'-O-(2-methoxyethyl)]-5-methyl-ribocytidine
  modified_base 18: mod_base = OTHER; note = [2'-O-(2-methoxyethyl)]-riboadenine
  modified_base 19: mod_base = OTHER; note = [2'-O-(2-methoxyethyl)]-riboguanine
  modified_base 20: mod_base = OTHER; note = [2'-O-(2-methoxyethyl)]-5-methyl-ribocytidine
atcccacgcccctgtccagc

SEQ ID NO: 41 - RNA, 21 nt, synthetic construct
  source 1..21: mol_type = other RNA
  modified_base 1: mod_base = OTHER; note = inverted 3'-3'-linked-abasic deoxyriboguanine-5'-phosphorothioate
  modified_base 2: mod_base = um
  modified_base 3: mod_base = gm
  modified_base 4: mod_base = gm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = cytosine 2'F ribonucleoside
  modified_base 10: mod_base = OTHER; note = uracil 2'F ribonucleoside
  modified_base 11: mod_base = OTHER; note = cytosine 2'F ribonucleoside
  modified_base 12: mod_base = um
  modified_base 13: mod_base = cm
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = um
  modified_base 20: mod_base = cm
  modified_base 21: mod_base = OTHER; note = inverted 3'-3'-linked-abasic deoxyribouridine-5'-phosphorothioate
gtggacttctctcaattttct

SEQ ID NO: 42 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-fluoroguanosine-3'-phosphorothioate
  modified_base 3: mod_base = OTHER; note = 2'-O-methylcitidine-3'-phosphorothioate
  modified_base 4: mod_base = OTHER; note = adenine 2'-F ribonucleoside
  modified_base 5: mod_base = OTHER; note = 2'-O-methyladenosine
  modified_base 6: mod_base = OTHER; note = adenine 2'-F ribonucleoside
  modified_base 7: mod_base = um
  modified_base 8: mod_base = OTHER; note = uracil 2'-F ribonucleoside
  modified_base 9: mod_base = gm
  modified_base 10: mod_base = OTHER; note = adenine 2'-F ribonucleoside
  modified_base 11: mod_base = um
  modified_base 12: mod_base = OTHER; note = guanine 2'-F ribonucleoside
  modified_base 13: mod_base = cm
  modified_base 14: mod_base = OTHER; note = cytosine 2'-F ribonucleoside
  modified_base 15: mod_base = um
  modified_base 16: mod_base = OTHER; note = adenine 2'-F ribonucleoside
  modified_base 17: mod_base = cm
  modified_base 18: mod_base = OTHER; note = adenine 2'-F ribonucleoside
  modified_base 19: mod_base = gm
  modified_base 20: mod_base = OTHER; note = 2'-O-methylcitidine-3'-phosphorothioate
  modified_base 21: mod_base = cm
agaaaattgagagaagtccac

SEQ ID NO: 43 - RNA, 21 nt, synthetic construct
  source 1..21: mol_type = other RNA
  modified_base 1: mod_base = OTHER; note = inverted 3'-3'-linked-abasic deoxyribocytidine-5'-phosphorothioate
  modified_base 2: mod_base = gm
  modified_base 3: mod_base = cm
  modified_base 4: mod_base = um
  modified_base 5: mod_base = gm
  modified_base 6: mod_base = um
  modified_base 7: mod_base = OTHER; note = 2'-O-methyladenosine
  modified_base 8: mod_base = gm
  modified_base 9: mod_base = OTHER; note = guanine 2'F ribonucleoside
  modified_base 10: mod_base = OTHER; note = cytosine 2'F ribonucleoside
  modified_base 11: mod_base = OTHER; note = adenine 2'F ribonucleoside
  modified_base 12: mod_base = um
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = um
  modified_base 17: mod_base = um
  modified_base 18: mod_base = gm
  modified_base 19: mod_base = gm
  modified_base 20: mod_base = um
  modified_base 21: mod_base = OTHER; note = inverted 3'-3'-linked-abasic deoxyriboadenosine-5'-phosphorothioate
cgctgtaggcataaattggta

SEQ ID NO: 44 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-fluoroadenosine-3'-phosphorothioate
  modified_base 3: mod_base = OTHER; note = 2'-O-methylcytidine-3'-phosphorothioate
  modified_base 4: mod_base = OTHER; note = cytosine 2'-F ribonucleoside
  modified_base 5: mod_base = OTHER; note = 2'-O-methyladenosine
  modified_base 6: mod_base = OTHER; note = adenine 2'-F ribonucleoside
  modified_base 7: mod_base = um
  modified_base 8: mod_base = OTHER; note = uracil 2'-F ribonucleoside
  modified_base 9: mod_base = um
  modified_base 10: mod_base = OTHER; note = adenine 2'-F ribonucleoside
  modified_base 11: mod_base = um
  modified_base 12: mod_base = OTHER; note = guanine 2'-F ribonucleoside
  modified_base 13: mod_base = cm
  modified_base 14: mod_base = OTHER; note = cytosine 2'-F ribonucleoside
  modified_base 15: mod_base = um
  modified_base 16: mod_base = OTHER; note = adenine 2'-F ribonucleoside
  modified_base 17: mod_base = cm
  modified_base 18: mod_base = OTHER; note = adenine 2'-F ribonucleoside
  modified_base 19: mod_base = gm
  modified_base 20: mod_base = OTHER; note = 2'-O-methylcytidine-3'-phosphorothioate
  modified_base 21: mod_base = gm
taccaatttatgcctacagcg

SEQ ID NO: 45 - DNA, 16 bp, synthetic construct
  source 1..16: mol_type = other DNA
  modified_base 1: mod_base = OTHER; note = 5-methyl cytosine 2'-b-D-MOE sugar moiety
  modified_base 2: mod_base = OTHER; note = guanine 2'-b-D-MOE sugar moiety
  modified_base 3: mod_base = OTHER; note = 5-methyl cytosine cEt sugar moiety
  modified_base 4: mod_base = OTHER; note = thymine 2'-b-D-deoxyribosyl sugar moiety
  modified_base 5: mod_base = OTHER; note = guanine 2'-b-D-deoxyribosyl sugar moiety
  modified_base 6: mod_base = OTHER; note = adenine 2'-b-D-deoxyribosyl sugar moiety
  modified_base 7: mod_base = OTHER; note = thymine 2'-b-D-deoxyribosyl sugar moiety
  modified_base 8: mod_base = OTHER; note = thymine 2'-b-D-deoxyribosyl sugar moiety
  modified_base 9: mod_base = OTHER; note = thymine 2'-b-D-deoxyribosyl sugar moiety
  modified_base 10: mod_base = OTHER; note = guanine 2'-b-D-deoxyribosyl sugar moiety
  modified_base 11: mod_base = OTHER; note = thymine 2'-b-D-deoxyribosyl sugar moiety
  modified_base 12: mod_base = OTHER; note = 5-methyl cytosine 2'-b-D-deoxyribosyl sugar moiety
  modified_base 13: mod_base = OTHER; note = 5-methyl cytosine 2'-b-D-deoxyribosyl sugar moiety
  modified_base 14: mod_base = OTHER; note = guanine cEt sugar moiety
  modified_base 15: mod_base = OTHER; note = guanine cEt sugar moiety
  modified_base 16: mod_base = OTHER; note = guanine 2'-b-D-MOE sugar moiety
cgctgatttgtccggg

SEQ ID NO: 46 - RNA, 17 nt, synthetic construct
  source 1..17: mol_type = other RNA
  modified_base 1: mod_base = gm
  modified_base 2: mod_base = um
  modified_base 3: mod_base = gm
  modified_base 4: mod_base = cm
  modified_base 5: mod_base = OTHER; note = 2'-deoxy-2'-fluoro-adenosine
  modified_base 6: mod_base = OTHER; note = 2'-deoxy-2'-fluoro-cytosine
  modified_base 7: mod_base = OTHER; note = 2'-deoxy-2'-fluoro-uridine
  modified_base 8: mod_base = um
  modified_base 9: mod_base = cm
  modified_base 10: mod_base = gm
  modified_base 11: mod_base = cm
  modified_base 12: mod_base = um
  modified_base 13: mod_base = um
  modified_base 14: mod_base = cm
  modified_base 15: mod_base = OTHER; note = 2'-O-methyladenosine
  modified_base 16: mod_base = cm
  modified_base 17: mod_base = OTHER; note = 2'-O-methyladenosine
gtgcacttcgcttcaca

SEQ ID NO: 47 - RNA, 22 nt, synthetic construct
  source 1..22: mol_type = other RNA
  modified_base 1: mod_base = um
  modified_base 2: mod_base = OTHER; note = 2'-deoxy-2'-fluoro-guanine
  modified_base 3: mod_base = um
  modified_base 4: mod_base = gm
  modified_base 5: mod_base = OTHER; note = unlocked adenine nucleic acid
  modified_base 6: mod_base = OTHER; note = 2'-O-methyladenosine
  modified_base 7: mod_base = gm
  modified_base 8: mod_base = cm
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = um
  modified_base 14: mod_base = OTHER; note = 2'-deoxy-2'-fluoro-guanine
  modified_base 15: mod_base = cm
  modified_base 16: mod_base = OTHER; note = 2'-deoxy-2'-fluoro-adenosine
  modified_base 17: mod_base = cm
  modified_base 18: mod_base = OTHER; note = 2'-O-methyladenosine
  modified_base 19: mod_base = cm
  modified_base 20: mod_base = gm
  modified_base 21: mod_base = gm
tgtgaagcgaagtgcacacggt

SEQ ID NO: 48 - RNA, 17 nt, synthetic construct
  source 1..17: mol_type = other RNA
  modified_base 1: mod_base = gm
  modified_base 2: mod_base = um
  modified_base 3: mod_base = gm
  modified_base 4: mod_base = cm
  modified_base 5: mod_base = OTHER; note = 2'-deoxy-2'-fluoro-adenosine
  modified_base 6: mod_base = OTHER; note = 2'-deoxy-2'-fluoro-cytosine
  modified_base 7: mod_base = OTHER; note = 2'-deoxy-2'-fluoro-uridine
  modified_base 8: mod_base = um
  modified_base 9: mod_base = cm
  modified_base 10: mod_base = gm
  modified_base 11: mod_base = cm
  modified_base 12: mod_base = um
  modified_base 13: mod_base = um
  modified_base 14: mod_base = cm
  modified_base 15: mod_base = OTHER; note = 2'-O-methyladenosine
  modified_base 16: mod_base = cm
  modified_base 17: mod_base = OTHER; note = 2'-O-methyladenosine
gtgcacttcgcttcaca

SEQ ID NO: 49 - RNA, 22 nt, synthetic construct
  source 1..22: mol_type = other RNA
  modified_base 1: mod_base = um
  modified_base 2: mod_base = OTHER; note = 2'-deoxy-2'-fluoro-guanine
  modified_base 3: mod_base = um
  modified_base 4: mod_base = gm
  modified_base 5: mod_base = OTHER; note = 2'-O-methyladenosine
  modified_base 6: mod_base = OTHER; note = unlocked adenine nucleic acid
  modified_base 7: mod_base = gm
  modified_base 8: mod_base = cm
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = um
  modified_base 14: mod_base = OTHER; note = 2'-deoxy-2'-fluoro-guanine
  modified_base 15: mod_base = cm
  modified_base 16: mod_base = OTHER; note = 2'-deoxy-2'-fluoro-adenosine
  modified_base 17: mod_base = cm
  modified_base 18: mod_base = OTHER; note = 2'-O-methyladenosine
  modified_base 19: mod_base = cm
  modified_base 20: mod_base = gm
  modified_base 21: mod_base = gm
tgtgaagcgaagtgcacacggt

SEQ ID NO: 50 - RNA, 17 nt, synthetic construct
  source 1..17: mol_type = other RNA
  modified_base 1: mod_base = gm
  modified_base 2: mod_base = um
  modified_base 3: mod_base = gm
  modified_base 4: mod_base = cm
  modified_base 5: mod_base = OTHER; note = 2'-deoxy-2'-fluoro-adenosine
  modified_base 6: mod_base = OTHER; note = 2'-deoxy-2'-fluoro-cytosine
  modified_base 7: mod_base = OTHER; note = 2'-deoxy-2'-fluoro-uridine
  modified_base 8: mod_base = um
  modified_base 9: mod_base = cm
  modified_base 10: mod_base = gm
  modified_base 11: mod_base = cm
  modified_base 12: mod_base = um
  modified_base 13: mod_base = um
  modified_base 14: mod_base = cm
  modified_base 15: mod_base = OTHER; note = 2'-O-methyladenosine
  modified_base 16: mod_base = cm
  modified_base 17: mod_base = OTHER; note = 2'-O-methyladenosine
gtgcacttcgcttcaca

SEQ ID NO: 51 - RNA, 22 nt, synthetic construct
  source 1..22: mol_type = other RNA
  modified_base 1: mod_base = um
  modified_base 2: mod_base = OTHER; note = 2'-deoxy-2'-fluoro-guanine
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = cm
  modified_base 9: mod_base = gm
  modified_base 10: mod_base = OTHER; note = 2'-O-methyladenosine
  modified_base 11: mod_base = OTHER; note = 2'-O-methyladenosine
  modified_base 12: mod_base = gm
  modified_base 13: mod_base = um
  modified_base 14: mod_base = OTHER; note = 2'-deoxy-2'-fluoro-guanine
  modified_base 15: mod_base = cm
  modified_base 16: mod_base = OTHER; note = 2'-deoxy-2'-fluoro-adenosine
  modified_base 17: mod_base = cm
  modified_base 18: mod_base = OTHER; note = 2'-O-methyladenosine
  modified_base 19: mod_base = cm
  modified_base 20: mod_base = gm
  modified_base 21: mod_base = gm
tgtgaagcgaagtgcacacggt

SEQ ID NO: 52 - protein, 14 aa, synthetic construct
  CROSSLNK 1: note = Interchain via Terminal Amine Group
  CROSSLNK 14: note = Interchain via Carboxy Terminal Group
SPSTPPTPSPSTPP

SEQ ID NO: 53 - protein, 14 aa, synthetic construct
  CROSSLNK 1: note = Interchain via Serine Hydroxy Group
  CROSSLNK 14: note = Interchain via Terminal Carboxyl Group
SPSTPPTPSPSTPP

SEQ ID NO: 54 - protein, 14 aa, synthetic construct
  CROSSLNK 3: note = Interchain via Serine Hydroxy Group
  CROSSLNK 14: note = Interchain via Carboxy Terminal Group
SPSTPPTPSPSTPP

SEQ ID NO: 55 - protein, 14 aa, synthetic construct
  CROSSLNK 4: note = Interchain via Threonine Hydroxy Group
  CROSSLNK 14: note = Interchain via Carboxy Terminal Group
SPSTPPTPSPSTPP

SEQ ID NO: 56 - protein, 14 aa, synthetic construct
  CROSSLNK 7: note = Interchain via Threonine Hydroxy Group
  CROSSLNK 14: note = Interchain via Carboxy Terminal Group
SPSTPPTPSPSTPP

SEQ ID NO: 57 - protein, 14 aa, synthetic construct
  CROSSLNK 9: note = Interchain via Serine Hydroxy Group
  CROSSLNK 14: note = Interchain via Carboxy Terminal Group
SPSTPPTPSPSTPP

SEQ ID NO: 58 - protein, 14 aa, synthetic construct
  CROSSLNK 11: note = Interchain via Serine Hydroxy Group
  CROSSLNK 14: note = Interchain via Carboxy Terminal Group
SPSTPPTPSPSTPP

SEQ ID NO: 59 - protein, 11 aa, synthetic construct
  MOD_RES 1: note = MeGly
  MOD_RES 2: note = MeGly
  MOD_RES 3: note = MeGly
  MOD_RES 4: note = MeGly
  MOD_RES 5: note = MeGly
  MOD_RES 6: note = MeGly
  MOD_RES 7: note = MeGly
  MOD_RES 8: note = MeGly
  MOD_RES 9: note = MeGly
  MOD_RES 10: note = MeGly
  MOD_RES 11: note = bAla
  CROSSLNK 1: note = Interchain via Terminal Amine Group
  CROSSLNK 11: note = Interchain via Terminal Carboxyl Group to diethoxyamino propylamide
GGGGGGGGGGA

SEQ ID NO: 60 - protein, 11 aa, synthetic construct
  MOD_RES 1: note = MeGly
  MOD_RES 2: note = MeGly
  MOD_RES 3: note = MeGly
  MOD_RES 4: note = MeGly
  MOD_RES 5: note = MeGly
  MOD_RES 6: note = MeGly
  MOD_RES 7: note = MeGly
  MOD_RES 8: note = MeGly
  MOD_RES 9: note = MeGly
  MOD_RES 10: note = MeGly
  MOD_RES 11: note = bAla
  CROSSLNK 1: note = Interchain via Terminal Amine Group to Ketone
  CROSSLNK 11: note = Interchain via Terminal Carboxyl Group to dimethoxy methyl
GGGGGGGGGGA

SEQ ID NO: 61 - protein, 11 aa, synthetic construct
  MOD_RES 1: note = MeGly
  MOD_RES 2: note = MeGly
  MOD_RES 3: note = MeGly
  MOD_RES 4: note = MeGly
  MOD_RES 5: note = MeGly
  MOD_RES 6: note = MeGly
  MOD_RES 7: note = MeGly
  MOD_RES 8: note = MeGly
  MOD_RES 9: note = MeGly
  MOD_RES 10: note = MeGly
  MOD_RES 11: note = bAla
  CROSSLNK 1: note = Interchain via Terminal Amine Group
  CROSSLNK 11: note = Interchain via Terminal Carboxyl Group to ethoxypropylamide
GGGGGGGGGGA

SEQ ID NO: 62 - protein, 11 aa, synthetic construct
  MOD_RES 1: note = MeGly
  MOD_RES 2: note = MeGly
  MOD_RES 3: note = MeGly
  MOD_RES 4: note = MeGly
  MOD_RES 5: note = MeGly
  MOD_RES 6: note = MeGly
  MOD_RES 7: note = MeGly
  MOD_RES 8: note = MeGly
  MOD_RES 9: note = MeGly
  MOD_RES 10: note = MeGly
  MOD_RES 11: note = bAla
  CROSSLNK 1: note = Interchain via Terminal Amine Group to Ketone
  CROSSLNK 11: note = Interchain via Terminal Carboxyl Group to 2-Amino ethoxy
GGGGGGGGGGA